(19) United States
(12) Patent Application Publication
(10) Pub. No.: US 2024/0398757 A1
(43) Pub. Date: Dec. 5, 2024

(54) INHIBITORS FOR CORONAVIRUSES

(71) Applicant: COCRYSTAL PHARMA, INC., Bothell, WA (US)

(72) Inventor: Irina C. Jacobson, Sammamish, WA (US)

(21) Appl. No.: 18/292,989

(22) PCT Filed: Aug. 3, 2022
(86) PCT No.: PCT/US2022/039233

§ 371 Date: Jan. 29, 2024

(51) Int. Cl. A61K 31/4015; A61K 31/45; A61K 31/454; C07D 207/26; C07D 211/76; C07D 401/12

(52) U.S. Cl. CPC A61K 31/4015; A61K 31/45; A61K 31/454; C07D 207/26; C07D 211/76; C07D 401/12

(57) ABSTRACT

Provided are compounds of Formula (I), Formula (Ia), Formula (Ib), and their use in methods of inhibiting the replication of noroviruses and coronaviruses in a biological sample or patient, of reducing the amount of noroviruses or coronaviruses in a biological sample or patient, and of treating norovirus and coronavirus in a patient, comprising administering to said biological sample or patient a safe and effective amount of a compound of Formula (I), Formula (Ia), Formula (Ib), or a pharmaceutically acceptable salt thereof.

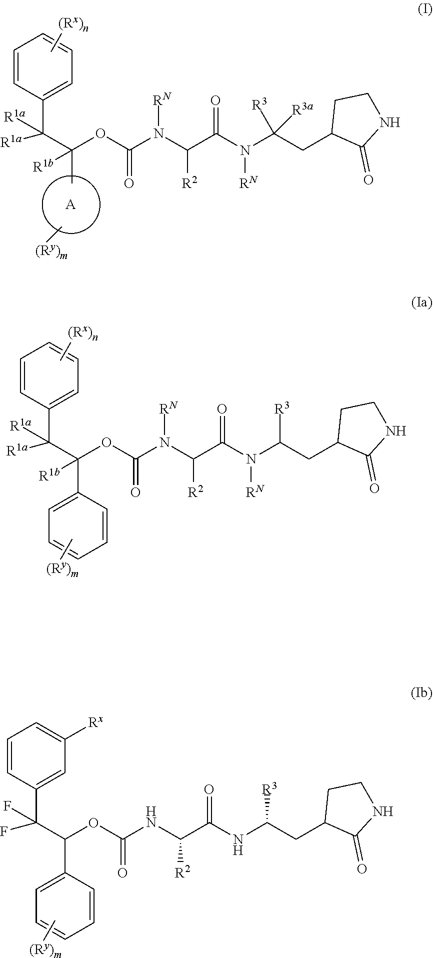

### FIELD OF THE DISCLOSURE

This disclosure relates generally to inhibitors of norovirus and coronavirus replication, and methods of treating or preventing norovirus and coronavirus infections by administering the inhibitors to a patient in need of treatment thereof.

### BACKGROUND

Noroviruses are important enteric pathogens involved in non-bacterial gastroenteritis outbreaks worldwide. Noroviruses mainly occur from person to person via the fecal-oral route but also through contaminated food or water. Indirect contamination is also possible owing to the persistence of the virus in the environment. Human noroviruses belong to the genus Norovirus, family Caliciviridae and are non-enveloped viruses with a positive-sense, single-stranded RNA genome. Norovirus strains are classified into seven groups. Viruses belonging to groups GI, GII, and GIV infect humans, while groups GII, GIII, GIV, GV, GVI and GVII NoVs have been described in animals.

Coronaviruses are a family of common viruses that cause a range of illnesses in humans from the common cold to severe acute respiratory syndrome (SARS). Coronaviruses can also cause a number of diseases in animals. Coronaviruses are enveloped, positive-stranded RNA viruses whose name derives from their characteristic crown-like appearance in electron micrographs. Coronaviruses are classified as a family within the Nidovirales order, viruses that replicate using a nested set of mRNAs. The coronavirus subfamily is further classified into four genera: alpha, beta, gamma, and delta coronaviruses. The human coronaviruses (HCoVs) are in two of these genera: alphacoronaviruses (including HCoV-229E and HCoV-NL63) and betacoronaviruses (including HCoV—HKU1, HCoV—OC43, Middle East respiratory syndrome coronavirus (MERS-CoV), the severe acute respiratory syndrome coronavirus (SARS-CoV), and SARS-CoV-2).

In 2012, a novel coronavirus emerged in Saudi Arabia and became known as Middle East Respiratory Syndrome coronavirus (MERS-CoV). About half of reported cases of MERS-CoV infection have resulted in death and a majority of reported cases have occurred in older to middle age men. Only a small number of reported cases involved subjects with mild respiratory illness. Human to human transmission of MERS-CoV has been found to be possible, but very limited. Another novel coronavirus emerged in Wuhan, China in late 2019. This virus is known as SARS-CoV-2, 2019-nCoV, or Wuhan coronavirus, and it the cause of a worldwide pandemic in late 2019 and 2020.

Given the widespread transmission and potential health effects of these viruses, there is a need for drugs for treating norovirus and coronavirus infections.

### SUMMARY

The present disclosure generally relates to methods of treating norovirus and coronavirus infections, to methods of inhibiting the replication of noroviruses and coronaviruses, to methods of reducing the amount of noroviruses and coronaviruses, and to compounds and compositions that can be employed for such methods.

The disclosure provides compounds and pharmaceutically acceptable salts thereof, wherein the compounds have a structure of Formula (I):

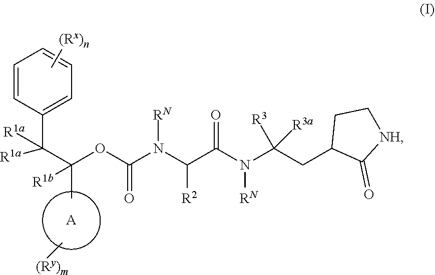

wherein each R^{N} is independently H or C₁₋₆alkyl; each R^{1a} is independently hydrogen, halo, C₁₋₆alkyl, or C₁₋₆haloalkyl, or both R^{1a} with the carbon to which they are attached form a spiro C₃₋₆carbocyclyl or spiro 4-8-membered heterocyclyl having 1-3 ring heteroatoms selected from N, O, and S, which is optionally substituted with C(O)OR^{N}; R^{1b} is hydrogen, halo, hydroxyl, C₁₋₆alkyl, or C₁₋₆haloalkyl; n is 0, 1, or 2; each R^{x} is independently halo, C₁₋₆alkyl, C₃₋₆carbocyclyl, or C₆₋₁₀aryl, wherein the aryl is optionally substituted with 1 or 2 substituents independently selected from OH, halo, C₁₋₆alkyl, C₁₋₆haloalkyl, and C₁₋₆alkoxy; m is 0, 1, or 2; each R^{y} is independently halo or C₁₋₆alkyl; R² is C₁₋₆alkyl, C₁₋₆haloalkyl, C₁₋₆alkylene-C₃₋₈carbocyclyl, or C₁₋₆alkylene-C₆₋₁₀aryl, wherein the aryl is optionally substituted with 1 or 2 substituents independently selected from OH, halo, C₁₋₆alkyl, C₁₋₆ haloalkyl, and C₁₋₆alkoxy; R³ is CN, C₁₋₆alkylene-O(O)C—C₁₋₆alkyl substituted with SO₃H, C₁₋₆alkenylene-C(O)O—C₁₋₆alkyl, C₁₋₆alkylene-OH substituted with PO(OCH₂CH₂)₂, CHO, or —[C(O)]₂—NR^{N}—B, wherein B is C₁₋₆alkyl, C₁₋₆hydroxyalkyl, C₃₋₈carbocyclyl, or 4-12-membered heterocyclyl having 1-3 ring heteroatoms selected from N, O, and S, and the carbocyclyl or heterocyclyl is optionally mono-substituted with C₁₋₆alkyl; R^{3a} is H or C₁₋₆alkyl; and ring A is C₆₋₁₀cycloalkyl, C₆₋₁₀aryl, or 5-10 membered heteroaryl comprising one nitrogen heteroatom, with the proviso that when each R^{1a} is hydrogen or each R^{1a} is methyl, ring A is phenyl, m is 0 or 1, R^{y} is halo, and n is 1, then R^{x} is other than chloro, and with the proviso that the compound is not: 1,2-diphenylethyl (4-methyl-1-oxo-1-((1-oxo-3-(2-oxopyrrolidin-3-yl)propan-2-yl)amino)pentan-2-yl)carbamate; 2-(2-(((1,2-diphenylethoxy)carbonyl)amino)-4-methylpentanamido)-1-hydroxy-3-(2-oxopyrrolidin-3-yl)propane-1-sulfonic acid; 2-(3-chlorophenyl)-1-phenylethyl (4-methyl-1-oxo-1-((1-oxo-3-(2-oxopyrrolidin-3-yl)propan-2-yl)amino)pentan-2-yl)carbamate; 2-(3-chlorophenyl)-1-(3-fluorophenyl)-2-methylpropyl (3-cyclohexyl-1-oxo-1-((1-oxo-3-(2-oxopyrrolidin-3-yl)propan-2-yl)amino)propan-2-yl)carbamate; 1,2-bis(3-chlorophenyl)-2-methylpropyl (3-cyclohexyl-1-oxo-1-((1-oxo-3-(2-oxopyrrolidin-3-yl)propan-2-yl)amino)propan-2-yl)carbamate; 2-(3-chlorophenyl)-2-methyl-1-phenylpropyl (4-methyl-1-oxo-1-((1-oxo-3-(2-oxopyrrolidin-3-yl)propan-2-yl)amino)pentan-2-yl)carbamate; 2-(3-chlorophenyl)-1-phenylethyl (3-cyclohexyl-1-oxo-1-((1-oxo-3-(2-oxopyrrolidin-3-yl)propan-2-yl)amino)propan-2-yl)carbamate; 2-(3-chlorophenyl)-2-methyl-1-phenylpropyl (3-cyclohexyl-1-oxo-1-((1-oxo-3-(2-oxopyrrolidin-3-yl)propan-2-yl)amino)propan-2-yl)carbamate; 2-(3-chlorophenyl)-2-methyl-1-phenylpropyl (1-((4-amino-3,4-dioxo-1-(2-oxopyrrolidin-3-yl)butan-2-yl)amino)-3-cyclohexyl-1-oxopropan-2-yl)carbamate; 2-(2-(((2-(3-chlorophenyl)-1-phenylethoxy)carbonyl)amino)-4-methylpentanamido)-1-hydroxy-3-(2-oxopyrrolidin-3-yl)propane-1-sulfonic acid; 2-(2-(((2-(3-chlorophenyl)-1-phenylethoxy)carbonyl)amino)-3-cyclohexylpropanamido)-1-hydroxy-3-(2-oxopyrrolidin-3-yl)propane-1-sulfonic acid; 2-(3-chlorophenyl)-1-phenylethyl (1-((4-amino-3,4-dioxo-1-(2-oxopyrrolidin-3-yl)butan-2-yl)amino)-3-cyclohexyl-1-oxopropan-2-yl)carbamate; 2-(3-chlorophenyl)-1-(3-fluorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate; 2-(3-chlorophenyl)-2-methyl-1-(naphthalen-2-yl)propyl (1-((4-amino-3,4-dioxo-1-(2-oxopyrrolidin-3-yl)butan-2-yl)amino)-3-cyclohexyl-1-oxopropan-2-yl)carbamate; 2-(3-chlorophenyl)-2-methyl-1-(m-tolyl)propyl (1-((4-amino-3,4-dioxo-1-(2-oxopyrrolidin-3-yl)butan-2-yl)amino)-3-cyclohexyl-1-oxopropan-2-yl)carbamate; 2-(3-chlorophenyl)-2-methyl-1-(naphthalen-2-yl)propyl (3-cyclohexyl-1-oxo-1-((1-oxo-3-(2-oxopyrrolidin-3-yl)propan-2-yl)amino)propan-2-yl)carbamate; 2-(3-chlorophenyl)-1-(4-chlorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate; 2-(3-chlorophenyl)-1-(4-fluorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate; 2-(3-chlorophenyl)-2-methyl-1-(m-tolyl)propyl (3-cyclohexyl-1-oxo-1-((1-oxo-3-(2-oxopyrrolidin-3-yl)propan-2-yl)amino)propan-2-yl)carbamate; 2-(3-chlorophenyl)-1-(4-fluorophenyl)-2-methylpropyl (3-cyclohexyl-1-oxo-1-((1-oxo-3-(2-oxopyrrolidin-3-yl)propan-2-yl)amino)propan-2-yl)carbamate; 2-(3-chlorophenyl)-1-(4-chlorophenyl)-2-methylpropyl (3-cyclohexyl-1-oxo-1-((1-oxo-3-(2-oxopyrrolidin-3-yl)propan-2-yl)amino)propan-2-yl)carbamate; 2-(3-chlorophenyl)-1-(4-fluorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate; 2-(3-chlorophenyl)-1-(4-chlorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate; 2-(3-chlorophenyl)-1-phenylethyl (1-((4-(cyclopropylamino)-3,4-dioxo-1-(2-oxopyrrolidin-3-yl)butan-2-yl)amino)-4-methyl-1-oxopentan-2-yl)carbamate; 2-(3-chlorophenyl)-2-methyl-1-phenylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate; 2-(3-chlorophenyl)-2-methyl-1-phenylpropyl (1-oxo-1-((1-oxo-3-(2-oxopyrrolidin-3-yl)propan-2-yl)amino)hexan-2-yl)carbamate; 2-(3-chlorophenyl)-1-phenylethyl (3-cyclohexyl-1-((4-(cyclopropylamino)-3,4-dioxo-1-(2-oxopyrrolidin-3-yl)butan-2-yl)amino)-1-oxopropan-2-yl)carbamate; 1-(2-chlorophenyl)-2-(3-chlorophenyl)-2-methylpropyl (3-cyclohexyl-1-oxo-1-((1-oxo-3-(2-oxopyrrolidin-3-yl)propan-2-yl)amino)propan-2-yl)carbamate; 2-(3-chlorophenyl)-2-methyl-1-phenylpropyl (1-((4-amino-3,4-dioxo-1-(2-oxopyrrolidin-3-yl)butan-2-yl)amino)-1-oxohexan-2-yl)carbamate; 2-(3-chlorophenyl)-2-methyl-1-phenylpropyl (1-((4-(cyclopropylamino)-3,4-dioxo-1-(2-oxopyrrolidin-3-yl)butan-2-yl)amino)-4-methyl-1-oxopentan-2-yl)carbamate; 2-(3-chlorophenyl)-2-methyl-1-phenylpropyl (1-((4-(cyclopropylamino)-3,4-dioxo-1-(2-oxopyrrolidin-3-yl)butan-2-yl)amino)-1-oxohexan-2-yl)carbamate; 2-(3-chlorophenyl)-1-(3-fluorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate; 2-(3-chlorophenyl)-1-(3-fluorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate; 1-(2-chlorophenyl)-2-(3-chlorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate; (3-chlorophenyl)(1-(3-chlorophenyl)cyclopropyl)methyl (1-((4-(cyclopropylamino)-3,4-dioxo-1-(2-oxopyrrolidin-3-yl)butan-2-yl)amino)-4-methyl-1-oxopentan-2-yl)carbamate; (4-chlorophenyl)(1-(3-chlorophenyl)cyclopropyl)methyl (4-methyl-1-oxo-1-((1-oxo-3-(2-oxopyrrolidin-3-yl)propan-2-yl)amino)pentan-2-yl)carbamate; (4-chlorophenyl)(1-(3-chlorophenyl)cyclopropyl)methyl (1-((4-(cyclopropylamino)-3,4-dioxo-1-(2-oxopyrrolidin-3-yl)butan-2-yl)amino)-4-methyl-1-oxopentan-2-yl)carbamate; (3-chlorophenyl)(1-(3-chlorophenyl)cyclopropyl)methyl (4-methyl-1-oxo-1-((1-oxo-3-(2-oxopyrrolidin-3-yl)propan-2-yl)amino)pentan-2-yl)carbamate; 2-(3-chlorophenyl)-2-methyl-1-phenylpropyl (1-((4-amino-3,4-dioxo-1-(2-oxopyrrolidin-3-yl)butan-2-yl)amino)-4-methyl-1-oxopentan-2-yl)carbamate; 2-(3-chlorophenyl)-2-methyl-1-phenylpropyl (3-cyclohexyl-1-((4-(ethylamino)-3,4-dioxo-1-(2-oxopyrrolidin-3-yl)butan-2-yl)amino)-1-oxopropan-2-yl)carbamate; (3-chlorophenyl)(1-(3-chlorophenyl)cyclopropyl)methyl (3-cyclohexyl-1-((4-(cyclopropylamino)-3,4-dioxo-1-(2-oxopyrrolidin-3-yl)butan-2-yl)amino)-1-oxopropan-2-yl)carbamate; 2-(3-chlorophenyl)-2-methyl-1-(naphthalen-2-yl)propyl (3-cyclohexyl-1-((4-(diethylamino)-3,4-dioxo-1-(2-oxopyrrolidin-3-yl)butan-2-yl)amino)-1-oxopropan-2-yl)carbamate; 2-(3-chlorophenyl)-1-(4-chlorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate; ethyl (E)-4-(2-(((1,2-diphenylethoxy)carbonyl)amino)-4-methylpentanamido)-5-(2-oxopyrrolidin-3-yl)pent-2-enoate; 3-(2-(((2-(3-chlorophenyl)-2-methyl-1-(naphthalen-2-yl)propoxy)carbonyl)amino)-3-cyclohexylpropanamio)-2-oxo-4-(2-oxopyrrolidin-3-yl)butanoic acid; 2-(3-chlorophenyl)-1-(4-fluorophenyl)-2-methylpropyl (4-methyl-1-oxo-1-((1-oxo-3-(2-oxopyrrolidin-3-yl)propan-2-yl)amino)pentan-2-yl)carbamate; 1,2-bis(3-chlorophenyl)ethyl (3-cyclohexyl-1-((4-(cyclopropylamino)-3,4-dioxo-1-(2-oxopyrrolidin-3-yl)butan-2-yl)amino)-1-oxopropan-2-yl)carbamate; 1,2-bis(3-chlorophenyl)ethyl (1-((4-(cyclopropylamino)-3,4-dioxo-1-(2-oxopyrrolidin-3-yl)butan-2-yl)amino)-4-methyl-1-oxopentan-2-yl)carbamate; 2-(3-chlorophenyl)-1-(4-chlorophenyl)-2-methylpropyl (4-methyl-1-oxo-1-((1-oxo-3-(2-oxopyrrolidin-3-yl)propan-2-yl)amino)pentan-2-yl)carbamate; 2-(3-chlorophenyl)-1-(4-fluorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate; (3-chlorophenyl)(1-(3-chlorophenyl)cyclopropyl)methyl (3-cyclohexyl-1-oxo-1-((1-oxo-3-(2-oxopyrrolidin-3-yl)propan-2-yl)amino)propan-2-yl)carbamate; 2-(3-chlorophenyl)-2,2-difluoro-1-phenylethyl (1-((4-amino-3,4-dioxo-1-(2-oxopyrrolidin-3-yl)butan-2-yl)amino)-4-methyl-1-oxopentan-2-yl)carbamate; 2-(3-chlorophenyl)-2,2-difluoro-1-phenylethyl (4-methyl-1-oxo-1-((1-oxo-3-(2-oxopyrrolidin-3-yl)propan-2-yl)amino)pentan-2-yl)carbamate; 2-(3-chlorophenyl)-2-methyl-1-phenylpropyl (1-((4-amino-3,4-dioxo-1-(2-oxopyrrolidin-3-yl)butan-2-yl)amino)-1-oxo-3-phenylpropan-2-yl)carbamate; 2-(3-chlorophenyl)-2-methyl-1-phenylpropyl (1-oxo-1-((1-oxo-3-(2-oxopyrrolidin-3-yl)propan-2-yl)amino)-3-phenylpropan-2-yl)carbamate; 2-(3-chlorophenyl)-2,2-difluoro-1-phenylethyl (1-((4-(cyclopropylamino)-3,4-dioxo-1-(2-oxopyrrolidin-3-yl)butan-2-yl)amino)-4-methyl-1-oxopentan-2-yl)carbamate; 1,2-bis(3-chlorophenyl)-2,2-difluoroethyl (4-methyl-1-oxo-1-((1-oxo-3-(2-oxopyrrolidin-3-yl)propan-2-yl)amino)pentan-2-yl)carbamate; 2-(3-chlorophenyl)-2-methyl-1-phenylpropyl (1-oxo-1-((1-oxo-3-(2-oxopyrrolidin-3-yl)propan-2-yl)amino)heptan-2-yl)carbamate; 2-(3-chlorophenyl)-1-(3-fluorophenyl)-2-methylpropyl (4-methyl-1-oxo-1-((1-oxo-3-(2-oxopyrrolidin-3-yl)propan-2-yl)amino)pentan-2-yl)carbamate; 1,2-bis(3-chlorophenyl)ethyl (3-cyclohexyl-1-oxo-1-((1-oxo-3-(2-oxopyrrolidin-3-yl)propan-2-yl)amino)propan-2-yl)carbamate; 2-(3-chlorophenyl)-2-methyl-1-phenylpropyl (4-methyl-1-oxo-1-((1-oxo-3-(2-oxopyrrolidin-3-yl)propan-2-yl)amino)pentan-2-yl)carbamate; (1-(3-chlorophenyl)cyclobutyl)(phenyl)methyl (4-methyl-1-oxo-1-((1-oxo-3-(2-oxopyrrolidin-3-yl)propan-2-yl)amino)pentan-2-yl)carbamate; (1-(3-chlorophenyl)cyclopentyl)(phenyl)methyl (4-methyl-1-oxo-1-((1-oxo-3-(2-oxopyrrolidin-3-yl)propan-2-yl)amino)pentan-2-yl)carbamate; 1,2-bis(3-chlorophenyl)ethyl (4-methyl-1-oxo-1-((1-oxo-3-(2-oxopyrrolidin-3-yl)propan-2-yl)amino)pentan-2-yl)carbamate; 2-(3-chlorophenyl)-2-methyl-1-phenylpropyl (1-((4-(cyclopropylamino)-3,4-dioxo-1-(2-oxopyrrolidin-3-yl)butan-2-yl)amino)-1-oxo-3-phenylpropan-2-yl)carbamate; 2-(3-chlorophenyl)-1-(3-fluorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate; 1,2-bis(3-chlorophenyl)-2,2-difluoroethyl (1-((4-(cyclopropylamino)-3,4-dioxo-1-(2-oxopyrrolidin-3-yl)butan-2-yl)amino)-4-methyl-1-oxopentan-2-yl)carbamate; 2-(3-chlorophenyl)-2-methyl-1-phenylpropyl (1-((4-(cyclopropylamino)-3,4-dioxo-1-(2-oxopyrrolidin-3-yl)butan-2-yl)amino)-1-oxoheptan-2-yl)carbamate; (1-(3-chlorophenyl)cyclohexyl)(phenyl)methyl (4-methyl-1-oxo-1-((1-oxo-3-(2-oxopyrrolidin-3-yl)propan-2-yl)amino)pentan-2-yl)carbamate; (1-(3-chlorophenyl)cyclopropyl)(4-fluorophenyl)methyl (1-((4-(cyclopropylamino)-3,4-dioxo-1-(2-oxopyrrolidin-3-yl)butan-2-yl)amino)-4-methyl-1-oxopentan-2-yl)carbamate; (4-chlorophenyl)(1-(3-chlorophenyl)cyclopentyl)methyl (4-methyl-1-oxo-1-((1-oxo-3-(2-oxopyrrolidin-3-yl)propan-2-yl)amino)pentan-2-yl)carbamate; (1-(3-chlorophenyl)cyclobutyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate; 2-(3-chlorophenyl)-2-ethyl-1-phenylbutyl (4-methyl-1-oxo-1-((1-oxo-3-(2-oxopyrrolidin-3-yl)propan-2-yl)amino)pentan-2-yl)carbamate; 2-(3-fluorophenyl)-2-methyl-1-phenylpropyl (1-((4-(cyclopropylamino)-3,4-dioxo-1-(2-oxopyrrolidin-3-yl)butan-2-yl)amino)-4-methyl-1-oxopentan-2-yl)carbamate; (1-(3-chlorophenyl)cyclopentyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate; (4-chlorophenyl)(1-(3-chlorophenyl)cyclopentyl)methyl (1-((4-(cyclopropylamino)-3,4-dioxo-1-(2-oxopyrrolidin-3-yl)butan-2-yl)amino)-4-methyl-1-oxopentan-2-yl)carbamate; 2-(3-chlorophenyl)-2-methyl-1-phenylpropyl (4-methyl-1-((4-((1-methylazetidin-3-yl)amino)-3,4-dioxo-1-(2-oxopyrrolidin-3-yl)butan-2-yl)amino)-1-oxopentan-2-yl)carbamate; 2-(3-chlorophenyl)-2-ethyl-1-(4-fluorophenyl)butyl (4-methyl-1-oxo-1-((1-oxo-3-(2-oxopyrrolidin-3-yl)propan-2-yl)amino)pentan-2-yl)carbamate; (3-chlorophenyl)(1-(3-chlorophenyl)cyclopentyl)methyl (4-methyl-1-oxo-1-((1-oxo-3-(2-oxopyrrolidin-3-yl)propan-2-yl)amino)pentan-2-yl)carbamate; (3-chlorophenyl)(1-(3-chlorophenyl)cyclopentyl)methyl (1-((4-(cyclopropylamino)-3,4-dioxo-1-(2-oxopyrrolidin-3-yl)butan-2-yl)amino)-4-methyl-1-oxopentan-2-yl)carbamate; 1,2-bis(3-chlorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate; (1-(3-chlorophenyl)cyclopropyl) (phenyl)methyl (1-((4-(cyclopropylamino)-3,4-dioxo-1-(2-oxopyrrolidin-3-yl)butan-2-yl)amino)-4-methyl-1-oxopentan-2-yl)carbamate; (1-(3-chlorophenyl)cyclopropyl)(4-fluorophenyl)methyl (4-methyl-1-oxo-1-((1-oxo-3-(2-oxopyrrolidin-3-yl)propan-2-yl)amino)pentan-2-yl)carbamate; (1-(3-fluorophenyl)cyclopropyl)(phenyl)methyl (4-methyl-1-oxo-1-((1-oxo-3-(2-oxopyrrolidin-3-yl)propan-2-yl)amino)pentan-2-yl)carbamate; (1-(3-fluorophenyl)cyclopropyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate; (4-chlorophenyl)(1-(3-chlorophenyl)cyclobutyl)methyl (4-methyl-1-oxo-1-((1-oxo-3-(2-oxopyrrolidin-3-yl)propan-2-yl)amino)pentan-2-yl)carbamate; or (4-chlorophenyl)(1-(3-chlorophenyl)cyclobutyl)methyl (1-((4-(cyclopropylamino)-3,4-dioxo-1-(2-oxopyrrolidin-3-yl)butan-2-yl)amino)-4-methyl-1-oxopentan-2-yl)carbamate.

Also provided are compounds and pharmaceutically acceptable salts thereof having a structure of Formula (Ia)

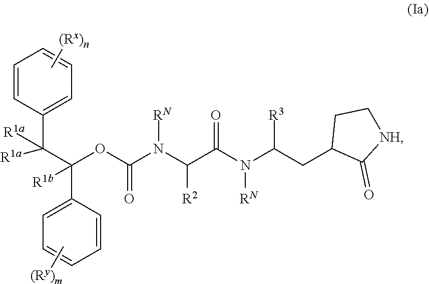

wherein each R^{N} is independently H or C₁₋₆alkyl; each R^{1a} is independently hydrogen, halo, C₁₋₆alkyl, or C₁₋₆haloalkyl, or both R^{1a} with the carbon to which they are attached form a spiro C₃₋₆carbocyclyl; R^{1b} is hydrogen, halo, C₁₋₆alkyl, or C₁₋₆ haloalkyl; n is 0, 1, or 2; each R^{x} is independently halo or C₁₋₆alkyl; m is 0, 1, or 2; each R^{y} is independently halo or C₁₋₆ alkyl; R² is C₁₋₆alkyl or C₁₋₆alkylene-C₅₋₈carbocyclyl; and R³ is CHO or —[C(O)]₂—NR^{N}—B, wherein B is C₁₋₆alkyl, C₃₋₈carbocyclyl, or 4-12-membered heterocyclyl having 1-3 ring heteroatoms selected from N, O, and S, and the carbocyclyl or heterocyclyl is optionally mono-substituted with C₁₋₆alkyl.

Also provided are compounds and pharmaceutically acceptable salts thereof having a structure of Formula (Ib)

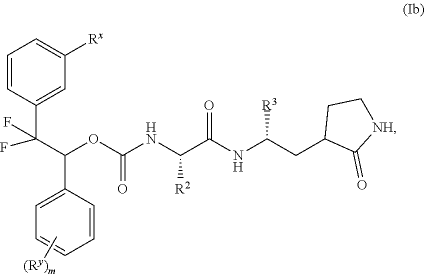

wherein R^{x} is Cl, F, cyclopropyl, or phenyl; R^{y} is F; m is 0 or 1; R² is C₄₋₅ alkyl, C₃₋₅haloalkyl, CH₂C₃₋₆carbocyclyl, or benzyl, and the carbocyclyl is optionally substituted with methyl or ethyl; and R³ is CHO, C(O)C(O)NH₂, C(O)C(O)NHcyclopropyl, or C(O)C(O)NHethyl, with the proviso that when R^{x} is Cl, m is 0, and R³ is CHO, C(O)C(O)NH₂, or C(O)C(O)NHcyclopropyl, then R² is not 2-methyl-propyl.

Further provided are methods of administering to a biological sample or patient a safe and effective amount of a compound as disclosed herein, e.g., a compound of Formula (I), (Ia), (Ib) or a pharmaceutically acceptable salt thereof.

Also provided herein are methods of reducing the amount of noroviruses or coronaviruses in a biological sample or in a patient by administering to said biological sample or patient a safe and effective amount of a compound as disclosed herein, e.g., a compound of Formula (I), (Ia), (Ib) or a pharmaceutically acceptable salt thereof.

Further provided are methods of treating or preventing a norovirus or coronavirus infection in a patient, comprising administering to said patient a safe and effective amount of a compound as disclosed herein, e.g., a compound of Formula (I), (Ia), (Ib) or a pharmaceutically acceptable salt thereof.

Also provided are pharmaceutical compositions comprising a compound as disclosed herein, e.g., a compound of Formula (I), (Ia), (Ib) or a pharmaceutically acceptable salt thereof, and a pharmaceutically acceptable excipient, carrier, adjuvant or vehicle.

Also provided are uses of a compound described herein for inhibiting or reducing the replication of noroviruses or coronaviruses in a biological sample or patient, for reducing the amount of noroviruses or coronaviruses in a biological sample or patient, or for treating norovirus or coronavirus in a patient.

Further provided herein are uses of a compound described herein for the manufacture of a medicament for treating a norovirus or coronavirus infection in a patient, for reducing the amount of noroviruses or coronaviruses in a biological sample or in a patient, or for inhibiting the replication of noroviruses or coronaviruses in a biological sample or patient.

### DETAILED DESCRIPTION

Provided herein are compounds, and their use in treating or preventing a viral infection (e.g., a norovirus or coronavirus infection). Also provided are uses of the compounds described herein, or pharmaceutically acceptable salts thereof, or pharmaceutically acceptable compositions comprising such a compound or a pharmaceutically acceptable salt thereof, for inhibiting the replication of viruses in a biological sample or in a patient, for reducing the amount of viruses (reducing viral titer) in a biological sample or in a patient, and for treating a viral infection in a patient.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

Unless otherwise indicated, all tautomeric forms of the compounds of the disclosure are within the scope of the disclosure.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this disclosure. Such compounds are useful, for example, as analytical tools or probes in biological assays. Such compounds, especially deuterium analogs, can also be therapeutically useful.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

### Compounds

Provided herein are compounds of Formula (I), and pharmaceutically acceptable salts thereof:

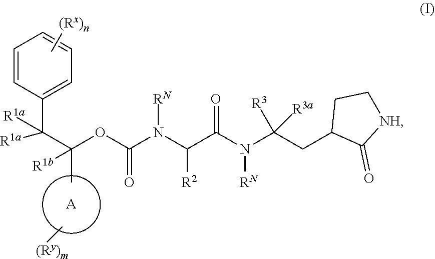

wherein
   each R^{N} is independently H or C₁₋₆alkyl;
   each R^{1a} is independently hydrogen, halo, C₁₋₆alkyl, or C₁₋₆haloalkyl,
   or both R^{1a} with the carbon to which they are attached form a spiro C₃₋₆carbocyclyl or spiro 4-8-membered heterocyclyl having 1-3 ring heteroatoms selected from N, O, and S, which is optionally substituted with C(O)OR^{N};
   R^{1b} is hydrogen, halo, hydroxyl, C₁₋₆alkyl, or C₁₋₆haloalkyl;
   n is 0, 1, or 2;
   each R^{x} is independently halo, C₁₋₆alkyl, C₃₋₆carbocyclyl, or C₆₋₁₀aryl, wherein the aryl is optionally substituted with 1 or 2 substituents independently selected from OH, halo, C₁₋₆alkyl, C₁₋₆haloalkyl, and C₁₋₆alkoxy;
m is 0, 1, or 2;
each R^{y} is independently halo or C₁₋₆alkyl;
   R² is C₁₋₆alkyl, C₁₋₆haloalkyl, C₁₋₆alkylene-C₃₋₆carbocyclyl, or C₁₋₆alkylene-C₆₋₁₀aryl, wherein the aryl is optionally substituted with 1 or 2 substituents independently selected from OH, halo, C₁₋₆alkyl, C₁₋₆haloalkyl, and C₁₋₆alkoxy;
   R³ is CN, C₁₋₆alkylene-O(O)C—C₁₋₆alkyl substituted with SO₃H, C₁₋₆alkenylene-C(O)O—C₁₋₆alkyl, C₁₋₆alkylene-OH substituted with PO(OCH₂CH₂)₂, CHO, or —[C(O)]₂—NR^{N}—B, wherein B is C₁₋₆alkyl, C₁₋₆hydroxyalkyl, C₃₋₆carbocyclyl, or 4-12-membered heterocyclyl having 1-3 ring heteroatoms selected from N, O, and S, and the carbocyclyl or heterocyclyl is optionally mono-substituted with C₁₋₆alkyl;
   R^{3a} is H or C₁₋₆alkyl; and
   ring A is C₆₋₁₀cycloalkyl, C₆₋₁₀aryl, or 5-10 membered heteroaryl comprising one nitrogen heteroatom,
with the proviso that when each R^{1a} is hydrogen or each R^{1a} is methyl, ring A is phenyl, m is 0 or 1, R^{y} is halo, and n is 1, then R^{x} is other than chloro, and
with the proviso that the compound is not:
1,2-diphenylethyl (4-methyl-1-oxo-1-((1-oxo-3-(2-oxopyrrolidin-3-yl)propan-2-yl)amino)pentan-2-yl)carbamate;
2-(2-(((1,2-diphenylethoxy)carbonyl)amino)-4-methylpentanamido)-1-hydroxy-3-(2-oxopyrrolidin-3-yl)propane-1-sulfonic acid;
2-(3-chlorophenyl)-1-phenylethyl (4-methyl-1-oxo-1-((1-oxo-3-(2-oxopyrrolidin-3-yl)propan-2-yl)amino)pentan-2-yl)carbamate;
2-(3-chlorophenyl)-1-(3-fluorophenyl)-2-methylpropyl (3-cyclohexyl-1-oxo-1-((1-oxo-3-(2-oxopyrrolidin-3-yl)propan-2-yl)amino)propan-2-yl)carbamate;
1,2-bis(3-chlorophenyl)-2-methylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (4-methyl-1-oxo-1-((1-oxo-3-(2-oxopyrrolidin-3-yl)propan-2-yl)amino)pentan-2-yl)carbamate;
2-(3-chlorophenyl)-1-phenylethyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (1-((4-amino-3,4-dioxo-1-(2-oxopyrrolidin-3-yl)butan-2-yl)amino)-3-cyclohexyl-1-oxopropan-2-yl)carbamate;
2-(2-(((2-(3-chlorophenyl)-1-phenylethoxy)carbonyl)amino)-4-methylpentanamido)-1-hydroxy-3-(2-oxopyrrolidin-3-yl)propane-1-sulfonic acid;
2-(2-(((2-(3-chlorophenyl)-1-phenylethoxy)carbonyl)amino)-3-cyclohexylpropanamido)-1-hydroxy-3-(2-oxopyrrolidin-3-yl)propane-1-sulfonic acid;
2-(3-chlorophenyl)-1-phenylethyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-1-(3-fluorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-2-methyl-1-(naphthalen-2-yl)propyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-2-methyl-1-(m-tolyl)propyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-2-methyl-1-(naphthalen-2-yl)propyl (3-cyclohexyl-1-oxo-1-((1-oxo-3-(2-oxopyrrolidin-3-yl)propan-2-yl)amino)propan-2-yl)carbamate;
2-(3-chlorophenyl)-1-(4-chlorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-1-(4-fluorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-2-methyl-1-(m-tolyl)propyl (3-cyclohexyl-1-oxo-1-((1-oxo-3-(2-oxopyrrolidin-3-yl)propan-2-yl)amino)propan-2-yl)carbamate;
2-(3-chlorophenyl)-1-(4-fluorophenyl)-2-methylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-1-(4-chlorophenyl)-2-methylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-1-(4-fluorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
2-(3-chlorophenyl)-1-(4-chlorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
2-(3-chlorophenyl)-1-phenylethyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
2-(3-chlorophenyl)-2-methyl-1-phenylpropyl (1-oxo-1-((1-oxo-3-(2-oxopyrrolidin-3-yl)propan-2-yl)amino)hexan-2-yl)carbamate;
2-(3-chlorophenyl)-1-phenylethyl (3-cyclohexyl-1-((4-(cyclopropylamino)-3,4-dioxo-1-(2-oxopyrrolidin-3-yl)butan-2-yl)amino)-1-oxopropan-2-yl)carbamate;
1-(2-chlorophenyl)-2-(3-chlorophenyl)-2-methylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (1-((4-amino-3,4-dioxo-1-(2-oxopyrrolidin-3-yl)butan-2-yl)amino)-1-oxohexan-2-yl)carbamate;
2-(3-chlorophenyl)-2-methyl-1-phenylpropyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (1-((4-(cyclopropylamino)-3,4-dioxo-1-(2-oxopyrrolidin-3-yl)butan-2-yl)amino)-1-oxohexan-2-yl)carbamate;
2-(3-chlorophenyl)-1-(3-fluorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-1-(3-fluorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
1-(2-chlorophenyl)-2-(3-chlorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
(3-chlorophenyl)(1-(3-chlorophenyl)cyclopropyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(4-chlorophenyl)(1-(3-chlorophenyl)cyclopropyl)methyl (4-methyl-1-oxo-1-((1-oxo-3-(2-oxopyrrolidin-3-yl)propan-2-yl)amino)pentan-2-yl)carbamate;
(4-chlorophenyl)(1-(3-chlorophenyl)cyclopropyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(3-chlorophenyl)(1-(3-chlorophenyl)cyclopropyl)methyl (4-methyl-1-oxo-1-((1-oxo-3-(2-oxopyrrolidin-3-yl)propan-2-yl)amino)pentan-2-yl)carbamate;
2-(3-chlorophenyl)-2-methyl-1-phenylpropyl (1-((4-amino-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (3-cyclohexyl-1-((4-(ethylamino)-3,4-dioxo-1-(2-oxopyrrolidin-3-yl)butan-2-yl)amino)-1-oxopropan-2-yl)carbamate;
(3-chlorophenyl)(1-(3-chlorophenyl)cyclopropyl)methyl (3-cyclohexyl-1-((4-(cyclopropylamino)-3,4-dioxo-1-(2-oxopyrrolidin-3-yl)butan-2-yl)amino)-1-oxopropan-2-yl)carbamate;
2-(3-chlorophenyl)-2-methyl-1-(naphthalen-2-yl)propyl (3-cyclohexyl-1-((4-(diethylamino)-3,4-dioxo-1-(2-oxopyrrolidin-3-yl)butan-2-yl)amino)-1-oxopropan-2-yl)carbamate;
2-(3-chlorophenyl)-1-(4-chlorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate;
ethyl (E)-4-(2-(((1,2-diphenylethoxy)carbonyl)amino)-4-methylpentanamido)-5-(2-oxopyrrolidin-3-yl)pent-2-enoate;
3-(2-(((2-(3-chlorophenyl)-2-methyl-1-(naphthalen-2-yl)propoxy)carbonyl)amino)-3-cyclohexylpropanamido)-2-oxo-4-(2-oxopyrrolidin-3-yl)butanoic acid;
2-(3-chlorophenyl)-1-(4-fluorophenyl)-2-methylpropyl (4-methyl-1-oxo-1-((1-oxo-3-(2-oxopyrrolidin-3-yl)propan-2-yl)amino)pentan-2-yl)carbamate;
1,2-bis(3-chlorophenyl)ethyl (3-cyclohexyl-1-((4-(cyclopropylamino)-3,4-dioxo-1-(2-oxopyrrolidin-3-yl)butan-2-yl)amino)-1-oxopropan-2-yl)carbamate;
1,2-bis(3-chlorophenyl)ethyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-1-(4-chlorophenyl)-2-methylpropyl (4-methyl-1-oxo-1-((1-oxo-3-(2-oxopyrrolidin-3-yl)propan-2-yl)amino)pentan-2-yl)carbamate;
2-(3-chlorophenyl)-1-(4-fluorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate;
(3-chlorophenyl)(1-(3-chlorophenyl)cyclopropyl)methyl (3-cyclohexyl-1-oxo-1-((1-oxo-3-(2-oxopyrrolidin-3-yl)propan-2-yl)amino)propan-2-yl)carbamate;
2-(3-chlorophenyl)-2,2-difluoro-1-phenylethyl (1-((4-amino-3,4-dioxo-1-(2-oxopyrrolidin-3-yl)butan-2-yl)amino)-4-methyl-1-oxopentan-2-yl)carbamate;
2-(3-chlorophenyl)-2,2-difluoro-1-phenylethyl (4-methyl-1-oxo-1-((1-oxo-3-(2-oxopyrrolidin-3-yl)propan-2-yl)amino)pentan-2-yl)carbamate;
2-(3-chlorophenyl)-2-methyl-1-phenylpropyl (1-((4-amino-3,4-dioxo-1-(2-oxopyrrolidin-3-yl)butan-2-yl)amino)-1-oxo-3-phenylpropan-2-yl)carbamate;
2-(3-chlorophenyl)-2-methyl-1-phenylpropyl (1-oxo-1-((1-oxo-3-(2-oxopyrrolidin-3-yl)propan-2-yl)amino)-3-phenylpropan-2-yl)carbamate;
2-(3-chlorophenyl)-2,2-difluoro-1-phenylethyl (1-((4-(cyclopropylamino)-3,4-dioxo-1-(2-oxopyrrolidin-3-yl)butan-2-yl)amino)-4-methyl-1-oxopentan-2-yl)carbamate;
1,2-bis(3-chlorophenyl)-2,2-difluoroethyl (4-methyl-1-oxo-1-((1-oxo-3-(2-oxopyrrolidin-3-yl)propan-2-yl)amino)pentan-2-yl)carbamate;
2-(3-chlorophenyl)-2-methyl-1-phenylpropyl (1-oxo-1-((1-oxo-3-(2-oxopyrrolidin-3-yl)propan-2-yl)amino)heptan-2-yl)carbamate;
2-(3-chlorophenyl)-1-(3-fluorophenyl)-2-methylpropyl (4-methyl-1-oxo-1-((1-oxo-3-(2-oxopyrrolidin-3-yl)propan-2-yl)amino)pentan-2-yl)carbamate;
1,2-bis(3-chlorophenyl)ethyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (4-methyl-1-oxo-1-((1-oxo-3-(2-oxopyrrolidin-3-yl)propan-2-yl)amino)pentan-2-yl)carbamate;
(1-(3-chlorophenyl)cyclobutyl)(phenyl)methyl (4-methyl-1-oxo-1-((1-oxo-3-(2-oxopyrrolidin-3-yl)propan-2-yl)amino)pentan-2-yl)carbamate;
(1-(3-chlorophenyl)cyclopentyl)(phenyl)methyl (4-methyl-1-oxo-1-((1-oxo-3-(2-oxopyrrolidin-3-yl)propan-2-yl)amino)pentan-2-yl)carbamate;
1,2-bis(3-chlorophenyl)ethyl (4-methyl-1-oxo-1-((1-oxo-3-(2-oxopyrrolidin-3-yl)propan-2-yl)amino)pentan-2-yl)carbamate;
2-(3-chlorophenyl)-2-methyl-1-phenylpropyl (1-((4-(cyclopropylamino)-3,4-dioxo-1-(2-oxopyrrolidin-3-yl)butan-2-yl)amino)-1-oxo-3-phenylpropan-2-yl)carbamate;
2-(3-chlorophenyl)-1-(3-fluorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate;
1,2-bis(3-chlorophenyl)-2,2-difluoroethyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (1-((4-(cyclopropylamino)-3,4-dioxo-1-(2-oxopyrrolidin-3-yl)butan-2-yl)amino)-1-oxoheptan-2-yl)carbamate;
(1-(3-chlorophenyl)cyclohexyl)(phenyl)methyl (4-methyl-1-oxo-1-((1-oxo-3-(2-oxopyrrolidin-3-yl)propan-2-yl)amino)pentan-2-yl)carbamate;
(1-(3-chlorophenyl)cyclopropyl)(4-fluorophenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(4-chlorophenyl)(1-(3-chlorophenyl)cyclopentyl)methyl (4-methyl-1-oxo-1-((1-oxo-3-(2-oxopyrrolidin-3-yl)propan-2-yl)amino)pentan-2-yl)carbamate;
(1-(3-chlorophenyl)cyclobutyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-ethyl-1-phenylbutyl (4-methyl-1-oxo-1-((1-oxo-3-(2-oxopyrrolidin-3-yl)propan-2-yl)amino)pentan-2-yl)carbamate;
2-(3-fluorophenyl)-2-methyl-1-phenylpropyl (1-((4-(cyclopropylamino)-3,4-dioxo-1-(2-oxopyrrolidin-3-yl)butan-2-yl)amino)-4-methyl-1-oxopentan-2-yl)carbamate;
(1-(3-chlorophenyl)cyclopentyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(4-chlorophenyl)(1-(3-chlorophenyl)cyclopentyl)methyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (4-methyl-1-((4-((1-methylazetidin-3-yl)amino)-3,4-dioxo-1-(2-oxopyrrolidin-3-yl)butan-2-yl)amino)-1-oxopentan-2-yl)carbamate;
2-(3-chlorophenyl)-2-ethyl-1-(4-fluorophenyl)butyl (4-methyl-1-oxo-1-((1-oxo-3-(2-oxopyrrolidin-3-yl)propan-2-yl)amino)pentan-2-yl)carbamate;
(3-chlorophenyl)(1-(3-chlorophenyl)cyclopentyl)methyl (4-methyl-1-oxo-1-((1-oxo-3-(2-oxopyrrolidin-3-yl)propan-2-yl)amino)pentan-2-yl)carbamate;
(3-chlorophenyl)(1-(3-chlorophenyl)cyclopentyl)methyl (1-((4-(cyclopropylamino)-3,4-dioxo-1-(2-oxopyrrolidin-3-yl)butan-2-yl)amino)-4-methyl-1-oxopentan-2-yl)carbamate;
1,2-bis(3-chlorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate;
(1-(3-chlorophenyl)cyclopropyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(1-(3-chlorophenyl)cyclopropyl)(4-fluorophenyl)methyl (4-methyl-1-oxo-1-((1-oxo-3-(2-oxopyrrolidin-3-yl)propan-2-yl)amino)pentan-2-yl)carbamate;
(1-(3-fluorophenyl)cyclopropyl)(phenyl)methyl (4-methyl-1-oxo-1-((1-oxo-3-(2-oxopyrrolidin-3-yl)propan-2-yl)amino)pentan-2-yl)carbamate;
(1-(3-fluorophenyl)cyclopropyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(4-chlorophenyl)(1-(3-chlorophenyl)cyclobutyl)methyl (4-methyl-1-oxo-1-((1-oxo-3-(2-oxopyrrolidin-3-yl)propan-2-yl)amino)pentan-2-yl)carbamate; or
(4-chlorophenyl)(1-(3-chlorophenyl)cyclobutyl)methyl (1-((4-(cyclopropylamino)-3,4-dioxo-1-(2-oxopyrrolidin-3-yl)butan-2-yl)amino)-4-methyl-1-oxopentan-2-yl)carbamate.

In some embodiments, the compounds and pharmaceutically acceptable salts thereof have a structure of Formula (Ia):

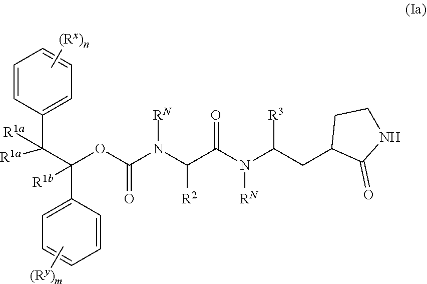

wherein
   each R^{N} is independently H or C₁₋₆alkyl;
   each R^{1a} is independently hydrogen, halo, C₁₋₆alkyl, or C₁₋₆haloalkyl,
   or both R^{1a} with the carbon to which they are attached form a spiro C₃₋₆carbocyclyl;
   R^{1b} is hydrogen, halo, C₁₋₆alkyl, or C₁₋₆haloalkyl;
   n is 0, 1, or 2;
   each R^{x} is independently halo or C₁₋₆alkyl;
   m is 0, 1, or 2;
   each R^{y} is independently halo or C₁₋₆alkyl;
R² is C₁₋₆alkyl or C₁₋₆alkylene-C₅₋₈carbocyclyl; and
   R³ is CHO or —[C(O)]₂—NR^{N}—B, wherein B is C₁₋₆alkyl, C₃₋₈carbocyclyl, or 4-12-membered heterocyclyl having 1-3 ring heteroatoms selected from N, O, and S, and the carbocyclyl or heterocyclyl is optionally mono-substituted with C₁₋₆ alkyl.

As used herein, the term “alkyl” or “alkylene” means a saturated straight or branched chain hydrocarbon. The term Cₙ means the alkyl group has “n” carbon atoms. For example, C₄alkyl refers to an alkyl group that has 4 carbon atoms. C₁₋₆alkyl refers to an alkyl group having a number of carbon atoms encompassing the entire range (i.e., 1 to 6 carbon atoms), as well as all subgroups (e.g., 1-6, 2-6, 1-5, 2-6, 1-4, 2-5, 1, 2, 3, 4, 5, and 6 carbon atoms). Specific examples include, but are not limited to, methyl, ethyl, isopropyl, n-propyl, sec-butyl, and t-butyl.

As used herein, the terms “halogen” and “halo” mean F, Cl, Br, or I.

As used herein, the term “haloalkyl” refers to an alkyl group in which one or more of the hydrogen atoms are replaced by halogen. Such groups include but are not limited to, chloromethyl, fluoromethyl, difluoromethyl, trifluoromethyl, 1,1-difluoroethyl, 2-fluoroethyl, 1-chloro-2-fluoromethyl and 2-fluoroisobutyl. A haloalkyl may be further substituted or unsubstituted, and some embodiments relate to a haloalkyl having e.g., 1 to 6 carbon atoms, such as C₁₋₆ haloalkyl.

The term “carbocycle” (or “carbocyclyl”) refers to a non-aromatic monocyclic, fused, bridged, or spiro ring system whose ring atoms are carbon and which can be saturated or have one or more units of unsaturation. The carbocycle can have 3 to 8 ring carbon atoms, such as from 3 to 6 ring carbon atoms. In some embodiments, the number of carbon atoms is 3 to 6, or 5 to 8. In some embodiments, the number of carbon atoms is 6. “Fused” bicyclic ring systems comprise two rings which share two adjoining ring atoms. Bridged bicyclic group comprise two rings which share three or four adjacent ring atoms. “Spiro” bicyclic ring systems share one ring atom. Specific examples include, but are not limited to, cyclohexyl, cyclopentyl, cyclopropyl, and cyclobutyl. A carbocycle ring is unsubstituted or substituted as described herein.

The term “heterocycle” (or “heterocyclyl”) as used herein refers to a non-aromatic monocyclic, fused, spiro or bridged ring system which can be saturated or contain one or more units of unsaturation, having 4 to 12 ring atoms in which one or more (e.g., one to three, or one, two, or three) ring atoms is a heteroatom selected from, N, S, and O. In some embodiments, the heterocycle comprises 5-6 ring members. In some embodiments, the heterocycle comprises 5 ring members. In some embodiments, the heterocycle comprises 6 ring members. In some embodiments, the heterocycle is piperidinyl. Examples of heterocycles include, but are not limited to, quinuclidinyl, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrothiophenyl, morpholino (including, for example, 3-morpholino, 4-morpholino), 2-thiomorpholino, 3-thiomorpholino, 4-thiomorpholino, 1-pyrrolidinyl, 2-pyrrolidinyl, 3-pyrrolidinyl, pyrrolidin-2-one, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolanyl, benzodithianyl, 3-(1-alkyl)-benzimidazol-2-onyl, and 1,3-dihydro-imidazol-2-onyl. A heterocycle ring is unsubstituted or substituted as described herein.

As described herein, compounds of the disclosure may optionally be substituted with one or more substituents, such as illustrated generally, or as exemplified by particular classes, subclasses, and species of the disclosure. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent.

Unless otherwise indicated, an optionally substituted group may have a substituent at each substitutable position of the group. When more than one position in a given structure can be substituted with more than one substituent selected from a specified group, the substituent may be either the same or different at each position.

In some cases, at least one R^{N} is H. In some cases, at least one R^{N} is C₁₋₆alkyl. In some cases, each R^{N} is H.

In some cases, at least one R^{N} is C₁₋₆alkyl, e.g., methyl. In some cases, each R^{N} is C₁₋₆alkyl, e.g., methyl.

In some cases, at least one R^{1a} is hydrogen. In some cases, each R^{1a} is hydrogen.

In some cases, at least one R^{1a} is halo. In some cases, each R^{1a} is halo. In some cases, halo is chloro or fluoro. In some cases, halo is fluoro. In some cases, at least one R^{1a} is fluoro. In some cases, each R^{1a} is fluoro. In some cases, at least one R^{1a} is chloro. In some cases, each R^{1a} is chloro.

In some cases, at least one R^{1a} is C₁₋₆alkyl. In some cases, each R^{1a} is C₁₋₆ alkyl. In some cases, C₁₋₆alkyl is methyl or ethyl. In some cases, at least one R^{1a} is methyl or ethyl. In some cases, each R^{1a} is methyl or ethyl. In some cases, each R^{1a} is methyl.

In some cases, each R^{1a}, together with the carbon to which they are attached, form a spiro C₃₋₆carbocyclyl. In some cases, the C₃₋₆carbocyclyl is cyclopropyl, cyclobutyl, cyclopentyl, or cyclohexyl. In some cases, the C₃. 6carbocyclyl is cyclopropyl. In some cases, the C₃₋₆carbocyclyl is cyclobutyl. In some cases, the C₃₋₆carbocyclyl is cyclopentyl. In some cases, the C₃₋₆carbocyclyl is cyclohexyl. In some cases, each R^{1a}, together with the carbon to which they are attached, form a spiro 4-8-membered heterocyclyl having 1-3 ring heteroatoms selected from N, O, and S, and is optionally substituted with C(O)OR^{N}. In some cases, each R^{1a}, together with the carbon to which they are attached, form a spiro piperidinyl. In some cases, each R^{1a}, together with the carbon to which they are attached, form an unsubstituted piperidinyl. In some cases, each R^{1a}, together with the carbon to which they are attached, form a spiro piperidinyl substituted with C(O)OR^{N}. In some cases, each R^{1a}, together with the carbon to which they are attached, form a spiro piperidinyl substituted with C(O)O-t-butyl.

In some cases, n is 0 or 1. In some cases, n is 0. In some cases, n is 1. In some cases, n is 2.

In some cases, R^{x} is in a meta or a para position. In some cases, R^{x} is in a meta position. In some cases, R^{x} is in a para position. In some cases, one R^{x} is in a meta position. In some cases, one R^{x} is in a para position. In some cases, one R^{x} is in a meta position and another R^{x} is in a para position. In some cases, at least one R^{x} is halo. In some cases, each R^{x} is halo. In some cases, at least one R^{x} is chloro. In some cases, at least one R^{x} is fluoro. In some cases, R^{x} is chloro. In some cases, R^{x} is fluoro. In some cases, each R^{x} is fluoro. In some cases, at least one R^{x} is C₁₋₆ alkyl. In some cases, two R^{x} are C₁₋₆alkyl. In some cases, at least one R^{x} is C₃₋₆carbocyclyl or C₆₋₁₀aryl, wherein the aryl is optionally substituted with 1 or 2 substituents independently selected from OH, halo, C₁₋₆alkyl, C₁₋₆haloalkyl, and C₁₋₆alkoxy. In some cases, at least one R^{x} is C₃₋₆carbocyclyl. In some cases, at least one R^{x} is cyclopropyl. In some cases, at least one R^{x} is C₆₋₁₀aryl, wherein the aryl is optionally substituted with 1 or 2 substituents independently selected from OH, halo, C₁₋₆alkyl, C₁₋₆haloalkyl, and C₁₋₆alkoxy. In some cases, at least one R^{x} is unsubstituted C₆₋₁₀aryl. In some cases, at least one R^{x} is phenyl. In some cases, at least one R^{x} is C₆₋₁₀aryl substituted with 1 or 2 substituents independently selected from OH, halo, C₁₋₆alkyl, C₁₋₆haloalkyl, and C₁₋₆alkoxy.

In some cases, m is 0 or 1. In some cases, m is 0. In some cases, m is 1. In some cases, m is 2.

In some cases, R^{y} is in a meta or a para position. In some cases, R^{y} is in a meta position. In some cases, R^{y} is in a para position. In some cases, one R^{y} is in a meta position. In some cases, one R^{y} is in a para position. In some cases, one R^{y} is in a meta position and another R^{y} is in a para position. In some cases, at least one R^{y} is halo. In some cases, each R^{y} is halo. In some cases, at least one R^{y} is chloro. In some cases, at least one R^{y} is fluoro. In some cases, R^{y} is chloro. In some cases, R^{y} is fluoro. In some cases, each R^{y} is fluoro. In some cases, at least one R^{y} is C₁₋₆ alkyl. In some cases, two R^{y} are C₁₋₆alkyl.

In some cases, R² is C₁₋₆alkyl. In some cases, R² is

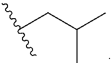 [SMILES: *CC(C)C]

In some cases, R² is C₁₋₆alkylene-C₃₋₈carbocyclyl. In some cases, R² is C₁₋₆alkylene-C₅₋₈carbocyclyl. In some cases, R² is C₁alkylene-C₅₋₈ carbocyclyl. In some cases, R² is

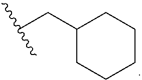 [SMILES: *CC1CCCCC1]

In some cases, R² is C₁₋₆haloalkyl, C₁₋₆alkylene-C₃₋₈carbocyclyl, or C₁₋₆alkylene-C₆₋₁₀aryl, wherein the aryl is optionally substituted with 1 or 2 substituents independently selected from OH, halo, C₁₋₆alkyl, C₁₋₆haloalkyl, and C₁₋₆alkoxy. In some cases, R² is C₁₋₆haloalkyl. In some cases, R² is C₁₋₆alkylene-C₆₋₁₀aryl. In some cases, R² is benzyl.

In some cases, R³ is CHO. In some cases, R³ is —[C(O)]₂—NR^{N}—B. In some cases, B is C₁₋₆alkyl or C₁₋₆ hydroxyalkyl. In some cases, B is C₁₋₆alkyl. In some cases, B is C₁₋₆hydroxyalkyl. In some cases, B is C₃₋₁₀ carbocyclyl optionally mono-substituted with C₁₋₆alkyl. In some cases, B is unsubstituted C₃₋₆carbocyclyl. In some cases, B is C₃₋₈carbocyclyl mono-substituted with C₁₋₆alkyl. In some cases, B is C₃carbocyclyl optionally mono-substituted with C₁₋₆ alkyl. In some cases, B is unsubstituted C₃carbocyclyl. In some cases, B is C₃carbocyclyl mono-substituted with C₁₋₆ alkyl. In some cases, B is unsubstituted C₆carbocyclyl. In some cases, B is C₆carbocyclyl mono-substituted with C₁₋₆ alkyl. In some cases, B is 4-12-membered heterocyclyl having 1-3 ring heteroatoms selected from N, O, and S optionally mono-substituted with C₁₋₆alkyl. In some cases, B is unsubstituted 4-12-membered heterocyclyl having 1-3 ring heteroatoms selected from N, O, and S. In some cases, B is 4-12-membered heterocyclyl having 1-3 ring heteroatoms selected from N, O, and S mono-substituted with C₁₋₆alkyl. In some cases, B comprises piperidinyl optionally mono-substituted with C₁₋₆alkyl. In some cases, B comprises unsubstituted piperidinyl. In some cases, B comprises piperidinyl mono-substituted with C₁₋₆alkyl. In some cases, B comprises piperidinyl mono-substituted with methyl. In some cases, B comprises piperidinyl mono-substituted with ethyl. In some cases, R³ is CN, C₁₋₆alkylene-O(O)C—C₁₋₆alkyl substituted with SO₃H, C₁₋₆alkenylene-C(O)O—C₁₋₆alkyl, or C₁₋₆alkylene-OH substituted with PO(OCH₂CH₂)₂.

In some cases, R^{3a} is H. In some cases, R^{3a} is C₁₋₆alkyl. In some cases, R^{3a} is methyl.

In some cases, ring A is C₆₋₁₀cycloalkyl. In some cases, ring A is cyclohexyl. In some cases, ring A is C₆₋₁₀aryl or 5-10 membered heteroaryl comprising one nitrogen heteroatom. In some cases, ring A is C₆₋₁₀aryl. In some cases, ring A is phenyl. In some cases, ring A is 5-10 membered heteroaryl comprising one nitrogen heteroatom. In some cases, ring A is pyridinyl.

In various cases, ring A comprises phenyl, each R^{1a} is F, R^{1b} is H, each R^{N} is H, n is 1, R^{x} is halo, R² is C₁₋₆ haloalkyl, C₁₋₆alkylene-C₃₋₈carbocyclyl, or C₁₋₆alkylene-C₆₋₁₀aryl, wherein the aryl is optionally substituted with 1 or 2 substituents independently selected from OH, halo, C₁₋₆alkyl, C₁₋₆haloalkyl, and C₁₋₆alkoxy; R³ is CN, C₁₋₆alkylene-O(O)C—C₁₋₆alkyl substituted with SO₃H, C₁₋₆alkenylene-C(O)O—C₁₋₆alkyl, C₁₋₆alkylene-OH substituted with PO(OCH₂CH₂)₂, CHO, or —[C(O)]₂—NR^{N}—B.

In some cases, the compounds and pharmaceutically acceptable salts thereof have a structure of Formula (Ib):

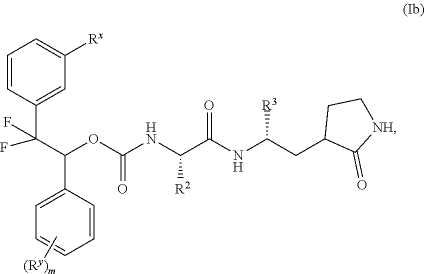

wherein
   R^{x} is Cl, F, cyclopropyl, or phenyl;
   R^{y} is F;
   m is 0 or 1;
   R² is C₄₋₅ alkyl, C₃₋₅haloalkyl, CH₂Cl₃₋₆carbocyclyl, or benzyl, and the carbocyclyl is optionally substituted with methyl or ethyl; and
   R³ is CHO, C(O)C(O)NH₂, C(O)C(O)NHcyclopropyl, or C(O)C(O)NHethyl,
with the proviso that when R^{x} is Cl, m is 0, and R³ is CHO, C(O)C(O)NH₂, or C(O)C(O)NHcyclopropyl, then R² is not 2-methyl-propyl.

In some cases, for the compound or salt of Formula (Ib), R^{x} is Cl. In some cases m is 0. In some cases, m is 1, and R^{y} is F (e.g., meta-F).

In some cases, for the compound or salt of Formula (b), R² is 2-methyl-propyl, butyl, pentyl, 2-methyl-butyl, 3,3-difluoropropyl, CH₂-cyclopropyl, CH₂cyclobutyl, CH₂cyclopentyl, CH₂-cyclohexyl, CH₂-(1-ethylcyclopropyl), CH₂-(1-methylcyclobutyl), CH₂-(l-ethyl cyclobutyl), CH₂-(1-ethylcyclopentyl), or benzyl. In some cases, R² is butyl, pentyl, 2-methyl-butyl, 3,3-difluoropropyl, CH₂-cyclopropyl, CH₂cyclobutyl, CH₂cyclopentyl, CH₂-cyclohexyl, CH₂-(1-ethylcyclopropyl), CH₂-(1-methylcyclobutyl), CH₂-(1-ethyl cyclobutyl), CH₂-(1-ethyl cyclopentyl), or benzyl. In some cases, R² is CH₂cyclohexyl. In some cases, R² is butyl or 2-methyl-butyl. In some cases, for the compound or salt of Formula (Ib), R³ is CHO. In some cases, R³ is C(O)C(O)NHcyclopropyl.

Specific compounds contemplated include compounds in the following Tables. Compounds showing particular stereocenters indicate at least a relative stereoisomerism. Compounds having a chiral center without indication of a particular stereoisomerism indicate a mixture of stereocenters at that chiral center.

The compound can be a compound as listed in Table A or Table B, or a pharmaceutically acceptable salt thereof.

TABLE A

| Compound | |
|---|---|
| no. | Structure |
| A1 | 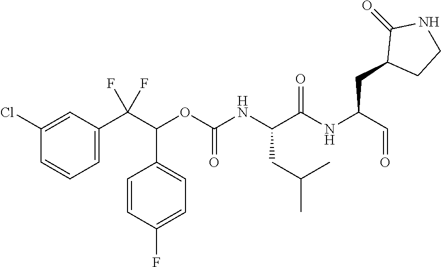 [SMILES: CC(C)C[C@H](NC(=O)OC(c1ccc(F)cc1)C(F)(F)c1cccc(Cl)c1)C(=O)N[C@H](C=O)C[C@@H]1CCNC1=O] |
| A2 | 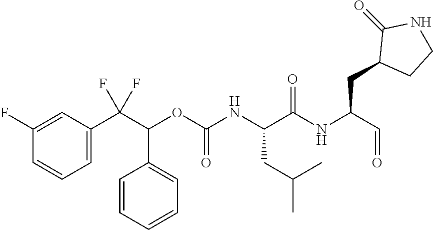 [SMILES: CC(C)C[C@H](NC(=O)OC(c1ccccc1)C(F)(F)c1cccc(F)c1)C(=O)N[C@H](C=O)C[C@@H]1CCNC1=O] |
| A3 | 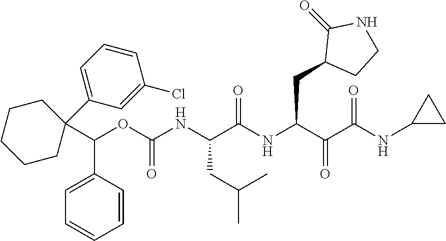 [SMILES: CC(C)C[C@H](NC(=O)OC(c1ccccc1)C1(c2cccc(Cl)c2)CCCCC1)C(=O)N[C@@H](C[C@@H]1CCNC1=O)C(=O)C(=O)NC1CC1] |
| A4 | 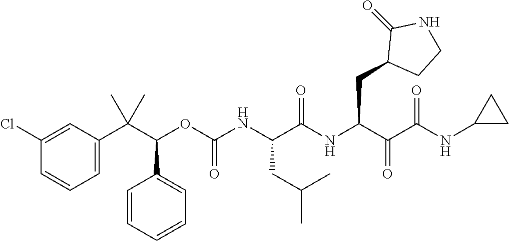 [SMILES: CC(C)C[C@H](NC(=O)O[C@@H](c1ccccc1)C(C)(C)c1cccc(Cl)c1)C(=O)N[C@@H](C[C@@H]1CCNC1=O)C(=O)C(=O)NC1CC1] |
| A5 | 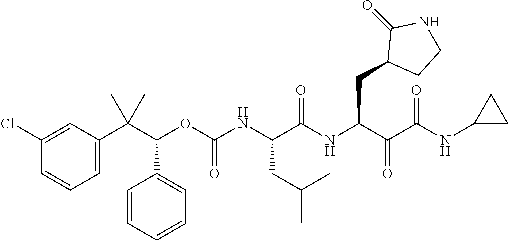 [SMILES: CC(C)C[C@H](NC(=O)O[C@H](c1ccccc1)C(C)(C)c1cccc(Cl)c1)C(=O)N[C@@H](C[C@@H]1CCNC1=O)C(=O)C(=O)NC1CC1] |
| A6 | 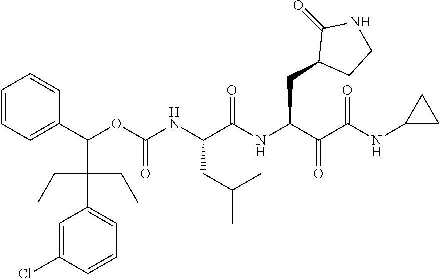 [SMILES: CCC(CC)(c1cccc(Cl)c1)C(OC(=O)N[C@@H](CC(C)C)C(=O)N[C@@H](C[C@@H]1CCNC1=O)C(=O)C(=O)NC1CC1)c1ccccc1] |
| A7 | 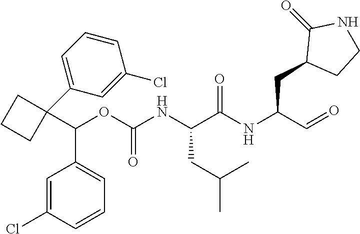 [SMILES: CC(C)C[C@H](NC(=O)OC(c1cccc(Cl)c1)C1(c2cccc(Cl)c2)CCC1)C(=O)N[C@H](C=O)C[C@@H]1CCNC1=O] |
| A9 | 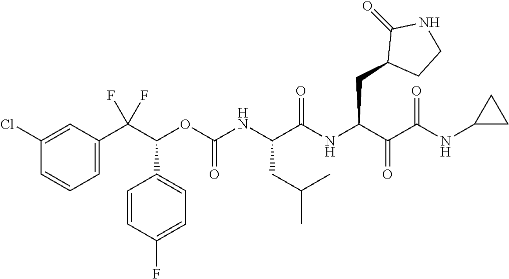 [SMILES: CC(C)C[C@H](NC(=O)O[C@H](c1ccc(F)cc1)C(F)(F)c1cccc(Cl)c1)C(=O)N[C@@H](C[C@@H]1CCNC1=O)C(=O)C(=O)NC1CC1] |
| A10 | 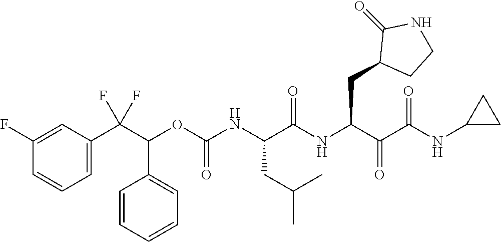 [SMILES: CC(C)C[C@H](NC(=O)OC(c1ccccc1)C(F)(F)c1cccc(F)c1)C(=O)N[C@@H](C[C@@H]1CCNC1=O)C(=O)C(=O)NC1CC1] |
| A11 | 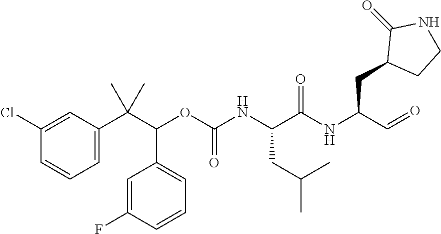 [SMILES: CC(C)C[C@H](NC(=O)OC(c1cccc(F)c1)C(C)(C)c1cccc(Cl)c1)C(=O)N[C@H](C=O)C[C@@H]1CCNC1=O] |
| A14 | 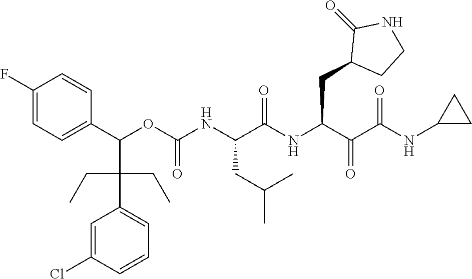 [SMILES: CCC(CC)(c1cccc(Cl)c1)C(OC(=O)N[C@@H](CC(C)C)C(=O)N[C@@H](C[C@@H]1CCNC1=O)C(=O)C(=O)NC1CC1)c1ccc(F)cc1] |
| A15 | 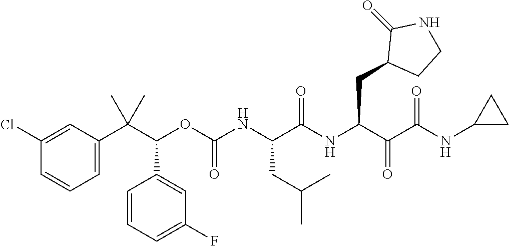 [SMILES: CC(C)C[C@H](NC(=O)O[C@H](c1cccc(F)c1)C(C)(C)c1cccc(Cl)c1)C(=O)N[C@@H](C[C@@H]1CCNC1=O)C(=O)C(=O)NC1CC1] |
| A16 | 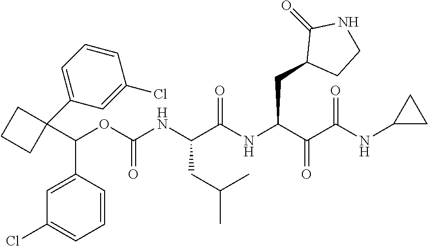 [SMILES: CC(C)C[C@H](NC(=O)OC(c1cccc(Cl)c1)C1(c2cccc(Cl)c2)CCC1)C(=O)N[C@@H](C[C@@H]1CCNC1=O)C(=O)C(=O)NC1CC1] |
| A17 | 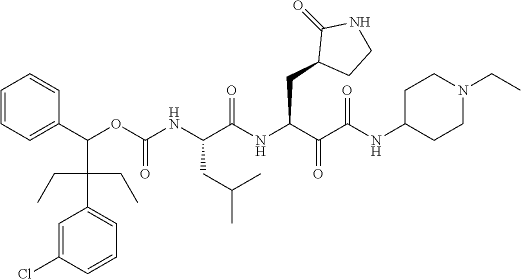 [SMILES: CCN1CCC(NC(=O)C(=O)[C@H](C[C@@H]2CCNC2=O)NC(=O)[C@H](CC(C)C)NC(=O)OC(c2ccccc2)C(CC)(CC)c2cccc(Cl)c2)CC1] |
| A18 | 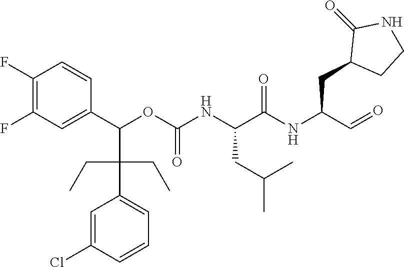 [SMILES: CCC(CC)(c1cccc(Cl)c1)C(OC(=O)N[C@@H](CC(C)C)C(=O)N[C@H](C=O)C[C@@H]1CCNC1=O)c1ccc(F)c(F)c1] |
| A19 | 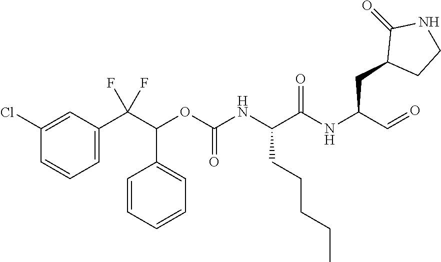 [SMILES: CCCCC[C@H](NC(=O)OC(c1ccccc1)C(F)(F)c1cccc(Cl)c1)C(=O)N[C@H](C=O)C[C@@H]1CCNC1=O] |
| A20 | 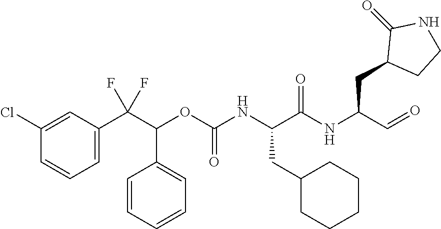 [SMILES: O=C[C@H](C[C@@H]1CCNC1=O)NC(=O)[C@H](CC1CCCCC1)NC(=O)OC(c1ccccc1)C(F)(F)c1cccc(Cl)c1] |
| A20-1 | 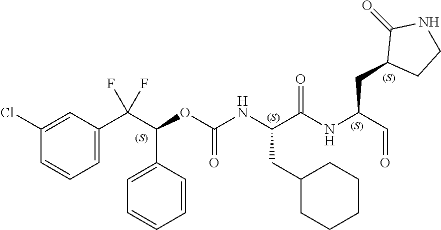 |
| A20-2 | 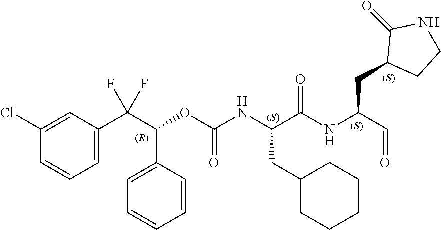 |
| A21 | 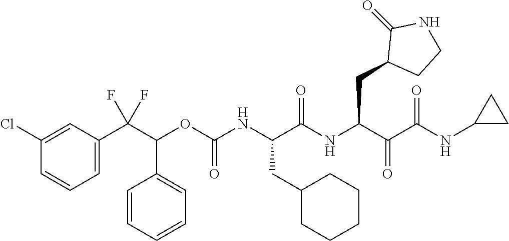 [SMILES: O=C(N[C@@H](CC1CCCCC1)C(=O)N[C@@H](C[C@@H]1CCNC1=O)C(=O)C(=O)NC1CC1)OC(c1ccccc1)C(F)(F)c1cccc(Cl)c1] |
| A22 | 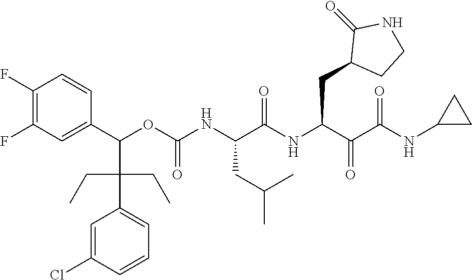 [SMILES: CCC(CC)(c1cccc(Cl)c1)C(OC(=O)N[C@@H](CC(C)C)C(=O)N[C@@H](C[C@@H]1CCNC1=O)C(=O)C(=O)NC1CC1)c1ccc(F)c(F)c1] |
| A25-1 | 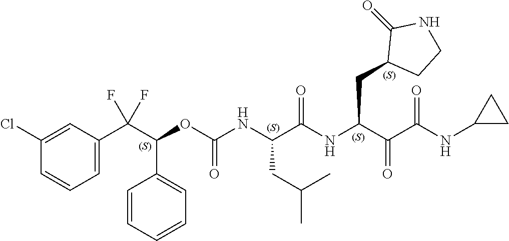 |
| A25-2 | 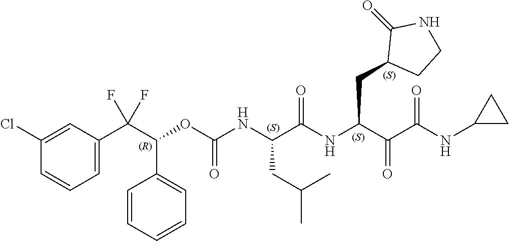 |
| A26P | 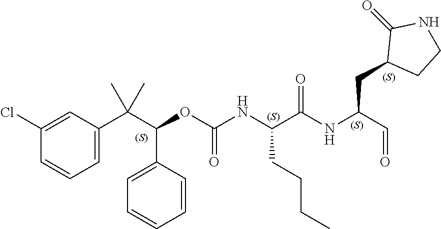 |
| A27 | 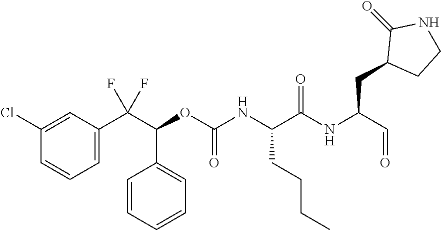 [SMILES: CCCC[C@H](NC(=O)O[C@@H](c1ccccc1)C(F)(F)c1cccc(Cl)c1)C(=O)N[C@H](C=O)C[C@@H]1CCNC1=O] |
| A29 | 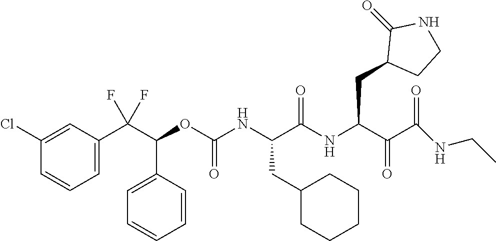 [SMILES: CCNC(=O)C(=O)[C@H](C[C@@H]1CCNC1=O)NC(=O)[C@H](CC1CCCCC1)NC(=O)O[C@@H](c1ccccc1)C(F)(F)c1cccc(Cl)c1] |
| A30 | 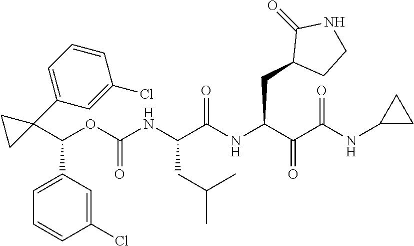 [SMILES: CC(C)C[C@H](NC(=O)O[C@H](c1cccc(Cl)c1)C1(c2cccc(Cl)c2)CC1)C(=O)N[C@@H](C[C@@H]1CCNC1=O)C(=O)C(=O)NC1CC1] |
| A31 | 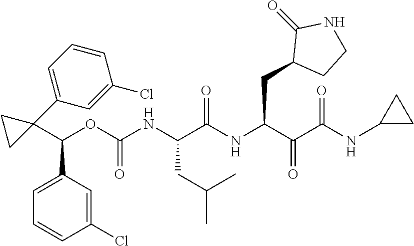 [SMILES: CC(C)C[C@H](NC(=O)O[C@@H](c1cccc(Cl)c1)C1(c2cccc(Cl)c2)CC1)C(=O)N[C@@H](C[C@@H]1CCNC1=O)C(=O)C(=O)NC1CC1] |
| A32 | 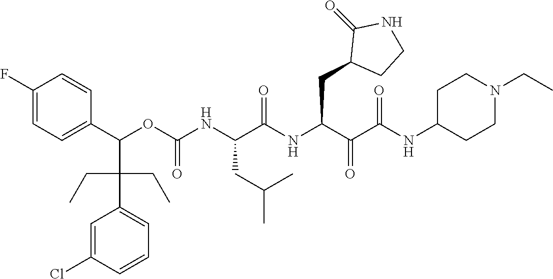 [SMILES: CCN1CCC(NC(=O)C(=O)[C@H](C[C@@H]2CCNC2=O)NC(=O)[C@H](CC(C)C)NC(=O)OC(c2ccc(F)cc2)C(CC)(CC)c2cccc(Cl)c2)CC1] |
| A33 | 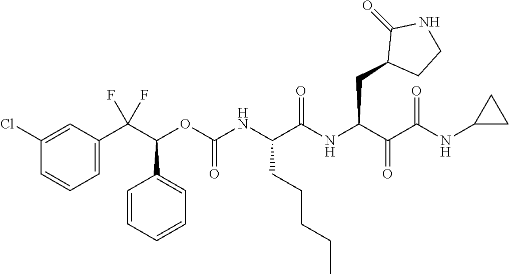 [SMILES: CCCCC[C@H](NC(=O)O[C@@H](c1ccccc1)C(F)(F)c1cccc(Cl)c1)C(=O)N[C@@H](C[C@@H]1CCNC1=O)C(=O)C(=O)NC1CC1] |
| A34 | 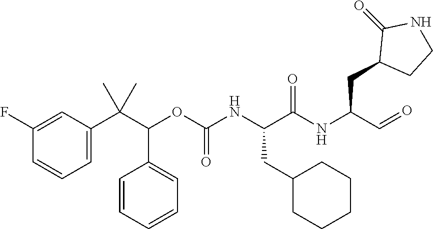 [SMILES: CC(C)(c1cccc(F)c1)C(OC(=O)N[C@@H](CC1CCCCC1)C(=O)N[C@H](C=O)C[C@@H]1CCNC1=O)c1ccccc1] |
| A35 | 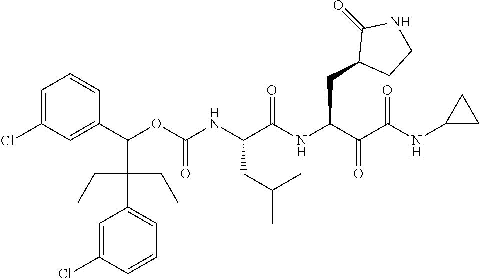 [SMILES: CCC(CC)(c1cccc(Cl)c1)C(OC(=O)N[C@@H](CC(C)C)C(=O)N[C@@H](C[C@@H]1CCNC1=O)C(=O)C(=O)NC1CC1)c1cccc(Cl)c1] |
| A36 | 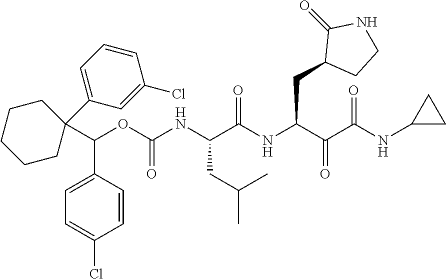 [SMILES: CC(C)C[C@H](NC(=O)OC(c1ccc(Cl)cc1)C1(c2cccc(Cl)c2)CCCCC1)C(=O)N[C@@H](C[C@@H]1CCNC1=O)C(=O)C(=O)NC1CC1] |
| A37 | 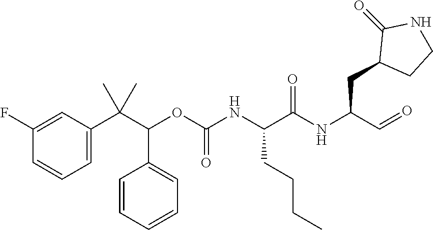 [SMILES: CCCC[C@H](NC(=O)OC(c1ccccc1)C(C)(C)c1cccc(F)c1)C(=O)N[C@H](C=O)C[C@@H]1CCNC1=O] |
| A38 | 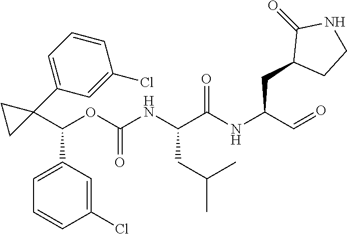 [SMILES: CC(C)C[C@H](NC(=O)O[C@H](c1cccc(Cl)c1)C1(c2cccc(Cl)c2)CC1)C(=O)N[C@H](C=O)C[C@@H]1CCNC1=O] |
| A39 | 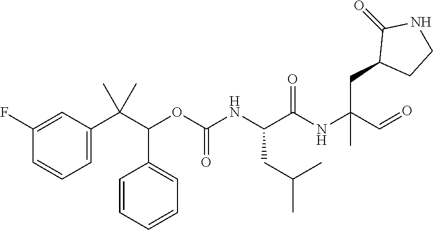 [SMILES: CC(C)C[C@H](NC(=O)OC(c1ccccc1)C(C)(C)c1cccc(F)c1)C(=O)NC(C)(C=O)C[C@@H]1CCNC1=O] |
| A40 | 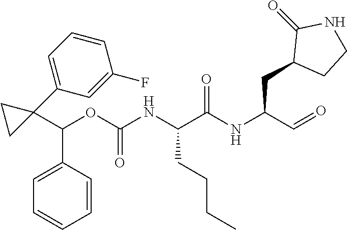 [SMILES: CCCC[C@H](NC(=O)OC(c1ccccc1)C1(c2cccc(F)c2)CC1)C(=O)N[C@H](C=O)C[C@@H]1CCNC1=O] |
| A41 | 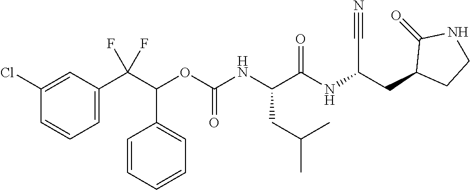 [SMILES: CC(C)C[C@H](NC(=O)OC(c1ccccc1)C(F)(F)c1cccc(Cl)c1)C(=O)N[C@H](C#N)C[C@@H]1CCNC1=O] |
| A42 | 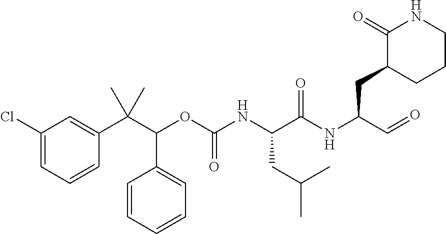 [SMILES: CC(C)C[C@H](NC(=O)OC(c1ccccc1)C(C)(C)c1cccc(Cl)c1)C(=O)N[C@H](C=O)C[C@@H]1CCCNC1=O] |
| A43 | 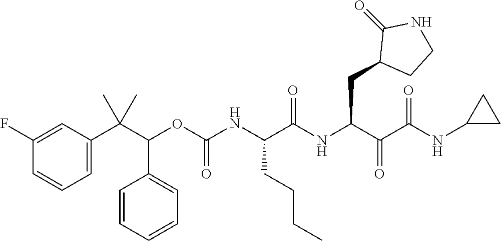 [SMILES: CCCC[C@H](NC(=O)OC(c1ccccc1)C(C)(C)c1cccc(F)c1)C(=O)N[C@@H](C[C@@H]1CCNC1=O)C(=O)C(=O)NC1CC1] |
| A44 | 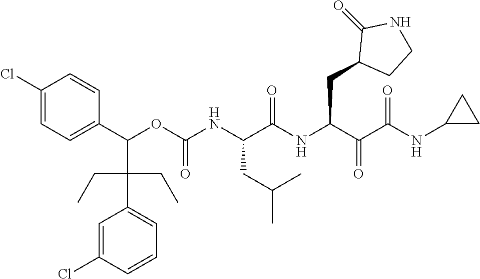 [SMILES: CCC(CC)(c1cccc(Cl)c1)C(OC(=O)N[C@@H](CC(C)C)C(=O)N[C@@H](C[C@@H]1CCNC1=O)C(=O)C(=O)NC1CC1)c1ccc(Cl)cc1] |
| A45 | 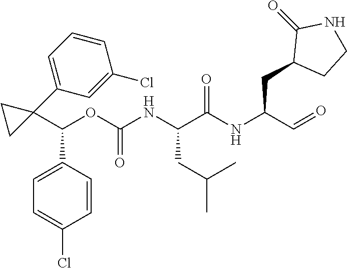 [SMILES: CC(C)C[C@H](NC(=O)O[C@H](c1ccc(Cl)cc1)C1(c2cccc(Cl)c2)CC1)C(=O)N[C@H](C=O)C[C@@H]1CCNC1=O] |
| A46 | 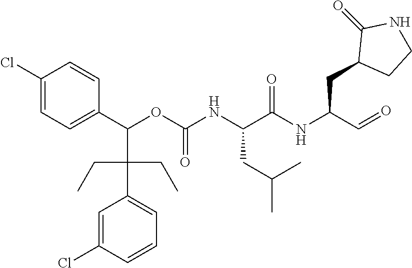 [SMILES: CCC(CC)(c1cccc(Cl)c1)C(OC(=O)N[C@@H](CC(C)C)C(=O)N[C@H](C=O)C[C@@H]1CCNC1=O)c1ccc(Cl)cc1] |
| A47 | 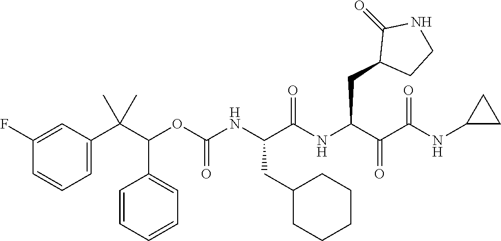 [SMILES: CC(C)(c1cccc(F)c1)C(OC(=O)N[C@@H](CC1CCCCC1)C(=O)N[C@@H](C[C@@H]1CCNC1=O)C(=O)C(=O)NC1CC1)c1ccccc1] |
| A48 | 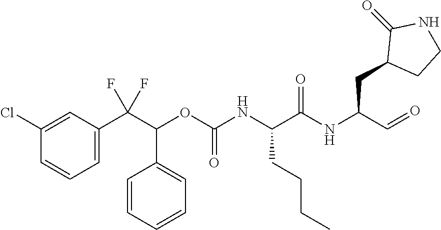 [SMILES: CCCC[C@H](NC(=O)OC(c1ccccc1)C(F)(F)c1cccc(Cl)c1)C(=O)N[C@H](C=O)C[C@@H]1CCNC1=O] |
| A49 | 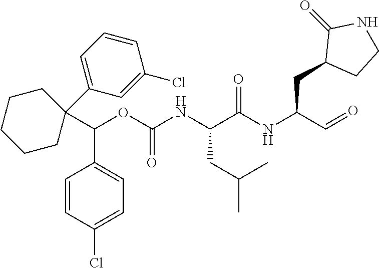 [SMILES: CC(C)C[C@H](NC(=O)OC(c1ccc(Cl)cc1)C1(c2cccc(Cl)c2)CCCCC1)C(=O)N[C@H](C=O)C[C@@H]1CCNC1=O] |
| A50 | 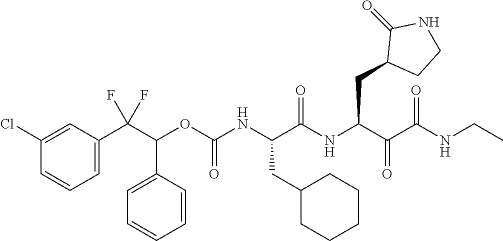 [SMILES: CCNC(=O)C(=O)[C@H](C[C@@H]1CCNC1=O)NC(=O)[C@H](CC1CCCCC1)NC(=O)OC(c1ccccc1)C(F)(F)c1cccc(Cl)c1] |
| A51 | 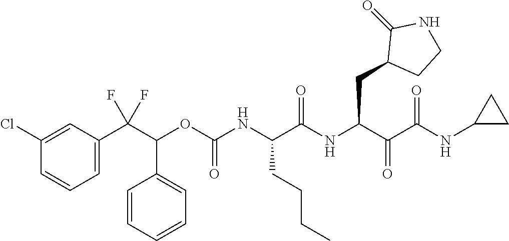 [SMILES: CCCC[C@H](NC(=O)OC(c1ccccc1)C(F)(F)c1cccc(Cl)c1)C(=O)N[C@@H](C[C@@H]1CCNC1=O)C(=O)C(=O)NC1CC1] |
| A52 | 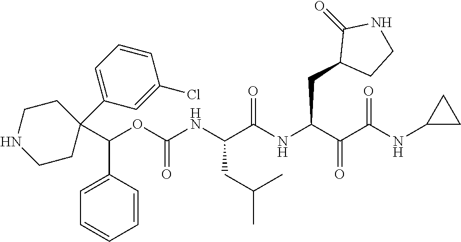 [SMILES: CC(C)C[C@H](NC(=O)OC(c1ccccc1)C1(c2cccc(Cl)c2)CCNCC1)C(=O)N[C@@H](C[C@@H]1CCNC1=O)C(=O)C(=O)NC1CC1] |
| A53 | 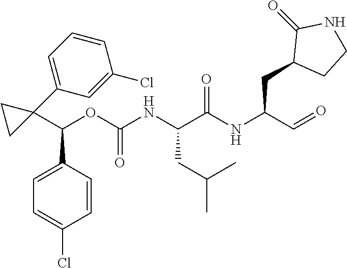 [SMILES: CC(C)C[C@H](NC(=O)O[C@@H](c1ccc(Cl)cc1)C1(c2cccc(Cl)c2)CC1)C(=O)N[C@H](C=O)C[C@@H]1CCNC1=O] |
| A54 | 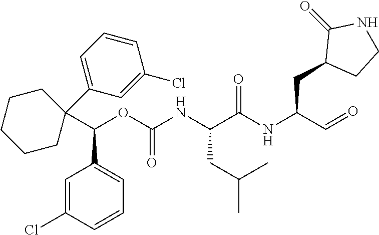 [SMILES: CC(C)C[C@H](NC(=O)O[C@@H](c1cccc(Cl)c1)C1(c2cccc(Cl)c2)CCCCC1)C(=O)N[C@H](C=O)C[C@@H]1CCNC1=O] |
| A55 | 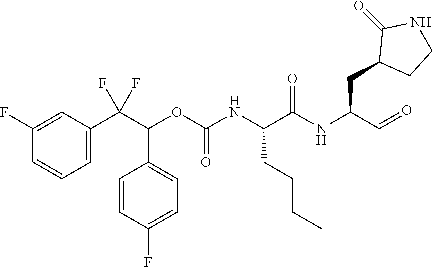 [SMILES: CCCC[C@H](NC(=O)OC(c1ccc(F)cc1)C(F)(F)c1cccc(F)c1)C(=O)N[C@H](C=O)C[C@@H]1CCNC1=O] |
| A56 | 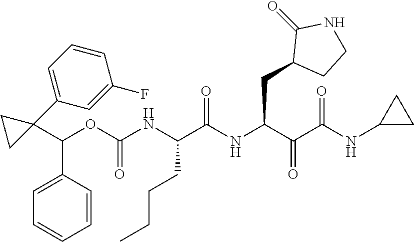 [SMILES: CCCC[C@H](NC(=O)OC(c1ccccc1)C1(c2cccc(F)c2)CC1)C(=O)N[C@@H](C[C@@H]1CCNC1=O)C(=O)C(=O)NC1CC1] |
| A57 | 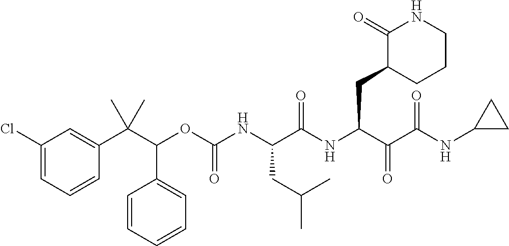 [SMILES: CC(C)C[C@H](NC(=O)OC(c1ccccc1)C(C)(C)c1cccc(Cl)c1)C(=O)N[C@@H](C[C@@H]1CCCNC1=O)C(=O)C(=O)NC1CC1] |
| A58 | 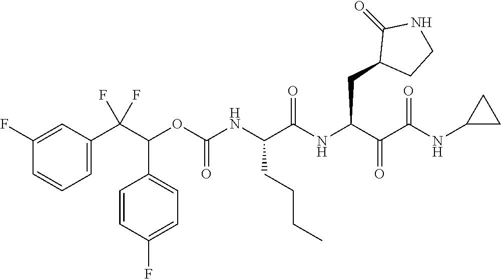 [SMILES: CCCC[C@H](NC(=O)OC(c1ccc(F)cc1)C(F)(F)c1cccc(F)c1)C(=O)N[C@@H](C[C@@H]1CCNC1=O)C(=O)C(=O)NC1CC1] |
| A59 | 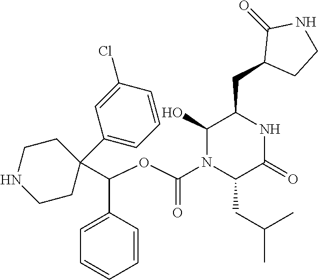 [SMILES: CC(C)C[C@H]1C(=O)N[C@H](C[C@@H]2CCNC2=O)[C@H](O)N1C(=O)OC(c1ccccc1)C1(c2cccc(Cl)c2)CCNCC1] |
| A60 | 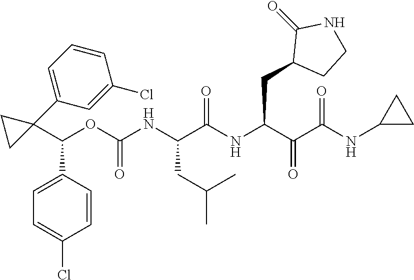 [SMILES: CC(C)C[C@H](NC(=O)O[C@H](c1ccc(Cl)cc1)C1(c2cccc(Cl)c2)CC1)C(=O)N[C@@H](C[C@@H]1CCNC1=O)C(=O)C(=O)NC1CC1] |
| A61 | 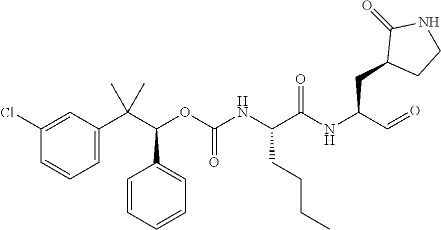 [SMILES: CCCC[C@H](NC(=O)O[C@@H](c1ccccc1)C(C)(C)c1cccc(Cl)c1)C(=O)N[C@H](C=O)C[C@@H]1CCNC1=O] |
| A62 | 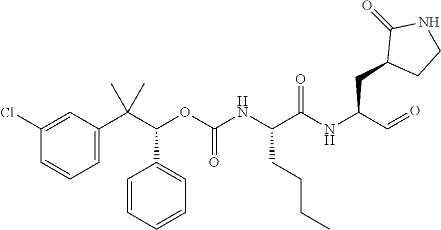 [SMILES: CCCC[C@H](NC(=O)O[C@H](c1ccccc1)C(C)(C)c1cccc(Cl)c1)C(=O)N[C@H](C=O)C[C@@H]1CCNC1=O] |
| A63 | 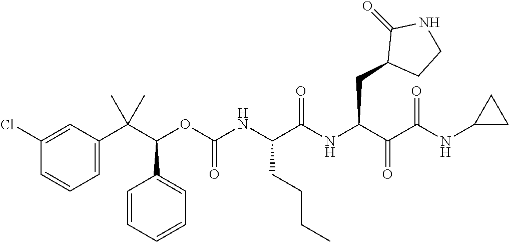 [SMILES: CCCC[C@H](NC(=O)O[C@@H](c1ccccc1)C(C)(C)c1cccc(Cl)c1)C(=O)N[C@@H](C[C@@H]1CCNC1=O)C(=O)C(=O)NC1CC1] |
| A64 | 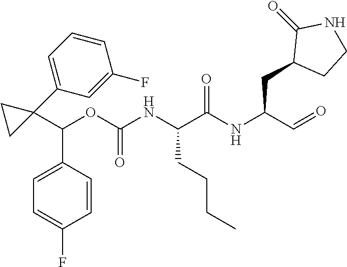 [SMILES: CCCC[C@H](NC(=O)OC(c1ccc(F)cc1)C1(c2cccc(F)c2)CC1)C(=O)N[C@H](C=O)C[C@@H]1CCNC1=O] |
| A65 | 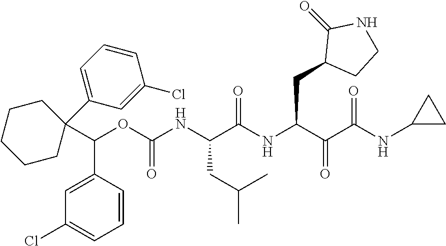 [SMILES: CC(C)C[C@H](NC(=O)OC(c1cccc(Cl)c1)C1(c2cccc(Cl)c2)CCCCC1)C(=O)N[C@@H](C[C@@H]1CCNC1=O)C(=O)C(=O)NC1CC1] |
| A66 | 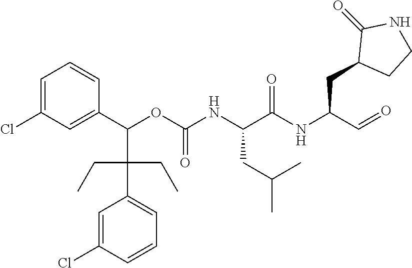 [SMILES: CCC(CC)(c1cccc(Cl)c1)C(OC(=O)N[C@@H](CC(C)C)C(=O)N[C@H](C=O)C[C@@H]1CCNC1=O)c1cccc(Cl)c1] |
| A67 | 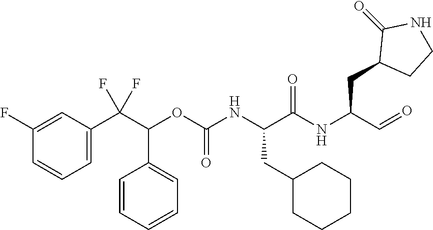 [SMILES: O=C[C@H](C[C@@H]1CCNC1=O)NC(=O)[C@H](CC1CCCCC1)NC(=O)OC(c1ccccc1)C(F)(F)c1cccc(F)c1] |
| A68 | 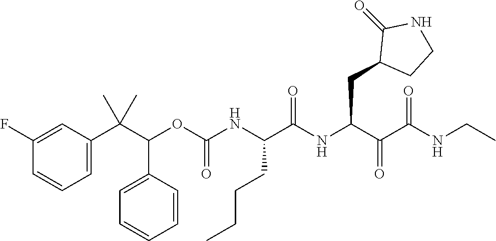 [SMILES: CCCC[C@H](NC(=O)OC(c1ccccc1)C(C)(C)c1cccc(F)c1)C(=O)N[C@@H](C[C@@H]1CCNC1=O)C(=O)C(=O)NCC] |
| A69 | 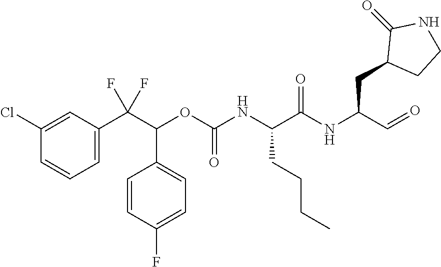 [SMILES: CCCC[C@H](NC(=O)OC(c1ccc(F)cc1)C(F)(F)c1cccc(Cl)c1)C(=O)N[C@H](C=O)C[C@@H]1CCNC1=O] |
| A70 | 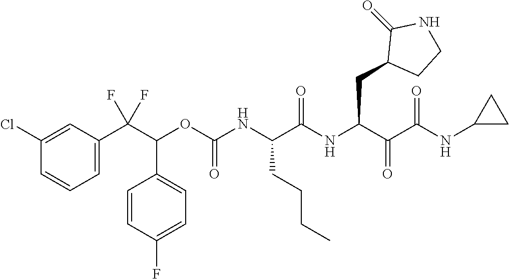 [SMILES: CCCC[C@H](NC(=O)OC(c1ccc(F)cc1)C(F)(F)c1cccc(Cl)c1)C(=O)N[C@@H](C[C@@H]1CCNC1=O)C(=O)C(=O)NC1CC1] |
| A71 | 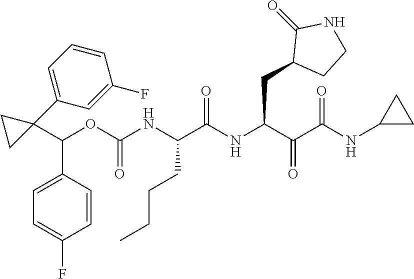 [SMILES: CCCC[C@H](NC(=O)OC(c1ccc(F)cc1)C1(c2cccc(F)c2)CC1)C(=O)N[C@@H](C[C@@H]1CCNC1=O)C(=O)C(=O)NC1CC1] |
| A72 | 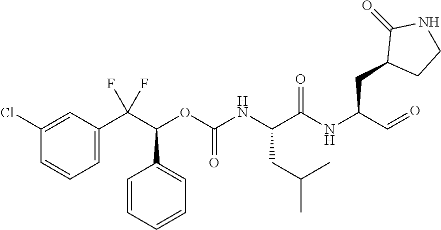 [SMILES: CC(C)C[C@H](NC(=O)O[C@@H](c1ccccc1)C(F)(F)c1cccc(Cl)c1)C(=O)N[C@H](C=O)C[C@@H]1CCNC1=O] |
| A73 | 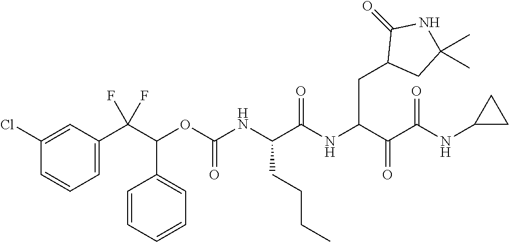 [SMILES: CCCC[C@H](NC(=O)OC(c1ccccc1)C(F)(F)c1cccc(Cl)c1)C(=O)NC(CC1CC(C)(C)NC1=O)C(=O)C(=O)NC1CC1] |
| A74 | 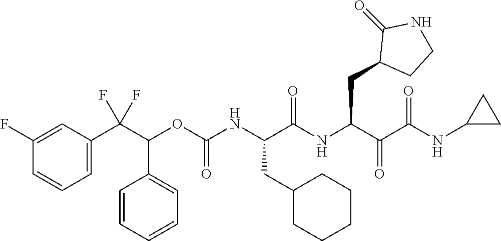 [SMILES: O=C(N[C@@H](CC1CCCCC1)C(=O)N[C@@H](C[C@@H]1CCNC1=O)C(=O)C(=O)NC1CC1)OC(c1ccccc1)C(F)(F)c1cccc(F)c1] |
| A75 | 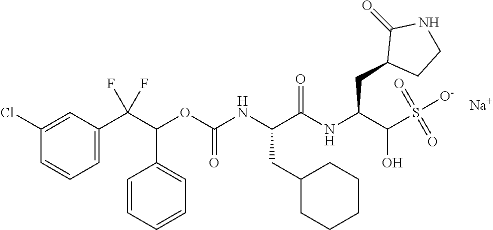 [SMILES: O=C(N[C@@H](CC1CCCCC1)C(=O)N[C@@H](C[C@@H]1CCNC1=O)C(O)S(=O)(=O)[O-])OC(c1ccccc1)C(F)(F)c1cccc(Cl)c1 ; [Na+]] |
| A76 | 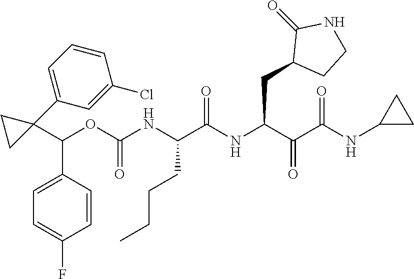 [SMILES: CCCC[C@H](NC(=O)OC(c1ccc(F)cc1)C1(c2cccc(Cl)c2)CC1)C(=O)N[C@@H](C[C@@H]1CCNC1=O)C(=O)C(=O)NC1CC1] |
| A77 | 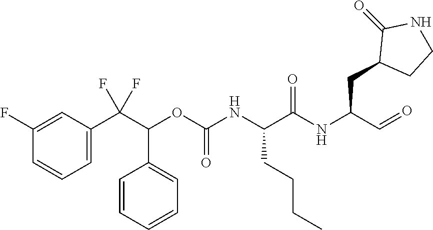 [SMILES: CCCC[C@H](NC(=O)OC(c1ccccc1)C(F)(F)c1cccc(F)c1)C(=O)N[C@H](C=O)C[C@@H]1CCNC1=O] |
| A78 | 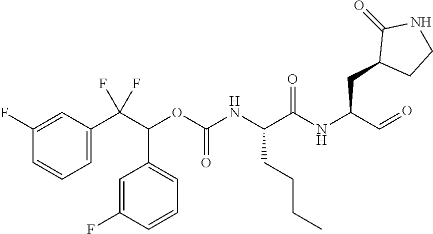 [SMILES: CCCC[C@H](NC(=O)OC(c1cccc(F)c1)C(F)(F)c1cccc(F)c1)C(=O)N[C@H](C=O)C[C@@H]1CCNC1=O] |
| A79 | 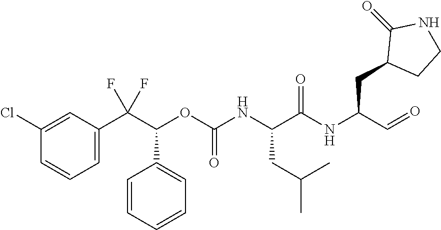 [SMILES: CC(C)C[C@H](NC(=O)O[C@H](c1ccccc1)C(F)(F)c1cccc(Cl)c1)C(=O)N[C@H](C=O)C[C@@H]1CCNC1=O] |
| A80 | 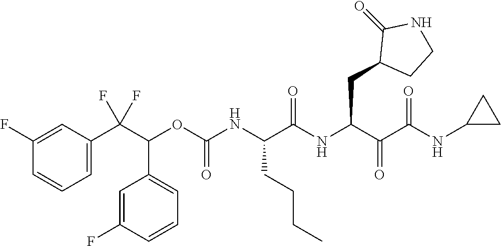 [SMILES: CCCC[C@H](NC(=O)OC(c1cccc(F)c1)C(F)(F)c1cccc(F)c1)C(=O)N[C@@H](C[C@@H]1CCNC1=O)C(=O)C(=O)NC1CC1] |
| A81 | 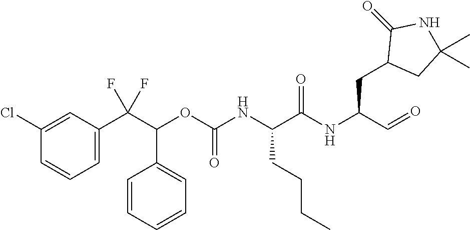 [SMILES: CCCC[C@H](NC(=O)OC(c1ccccc1)C(F)(F)c1cccc(Cl)c1)C(=O)N[C@H](C=O)CC1CC(C)(C)NC1=O] |
| A83 | 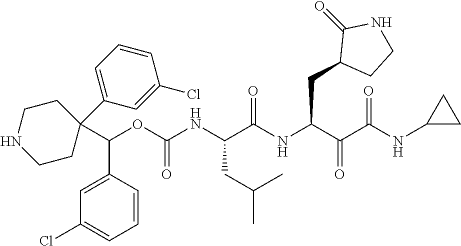 [SMILES: CC(C)C[C@H](NC(=O)OC(c1cccc(Cl)c1)C1(c2cccc(Cl)c2)CCNCC1)C(=O)N[C@@H](C[C@@H]1CCNC1=O)C(=O)C(=O)NC1CC1] |
| A84 | 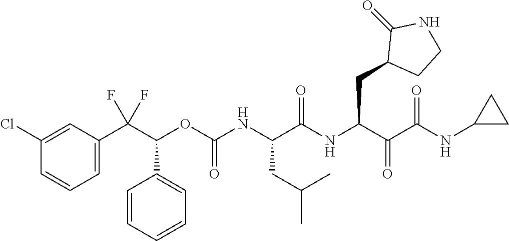 [SMILES: CC(C)C[C@H](NC(=O)O[C@H](c1ccccc1)C(F)(F)c1cccc(Cl)c1)C(=O)N[C@@H](C[C@@H]1CCNC1=O)C(=O)C(=O)NC1CC1] |
| A85 | 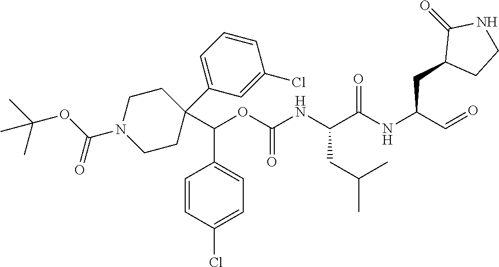 [SMILES: CC(C)C[C@H](NC(=O)OC(c1ccc(Cl)cc1)C1(c2cccc(Cl)c2)CCN(C(=O)OC(C)(C)C)CC1)C(=O)N[C@H](C=O)C[C@@H]1CCNC1=O] |
| A86 | 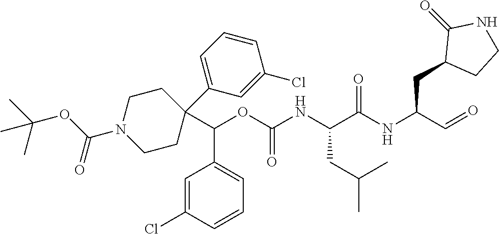 [SMILES: CC(C)C[C@H](NC(=O)OC(c1cccc(Cl)c1)C1(c2cccc(Cl)c2)CCN(C(=O)OC(C)(C)C)CC1)C(=O)N[C@H](C=O)C[C@@H]1CCNC1=O] |
| A87 | 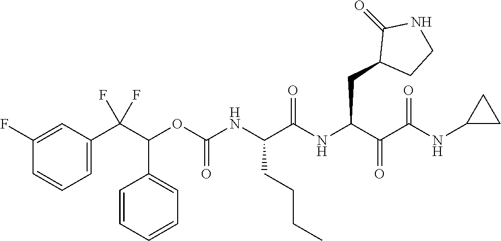 [SMILES: CCCC[C@H](NC(=O)OC(c1ccccc1)C(F)(F)c1cccc(F)c1)C(=O)N[C@@H](C[C@@H]1CCNC1=O)C(=O)C(=O)NC1CC1] |
| A88 | 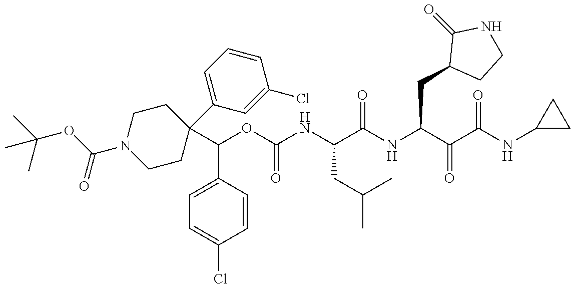 [SMILES: CC(C)C[C@H](NC(=O)OC(c1ccc(Cl)cc1)C1(c2cccc(Cl)c2)CCN(C(=O)OC(C)(C)C)CC1)C(=O)N[C@@H](C[C@@H]1CCNC1=O)C(=O)C(=O)NC1CC1] |
| A89 | 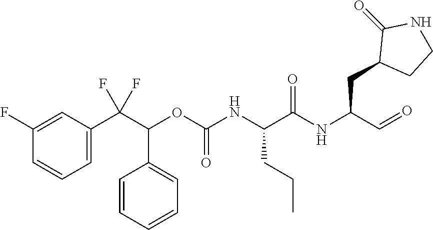 [SMILES: CCC[C@H](NC(=O)OC(c1ccccc1)C(F)(F)c1cccc(F)c1)C(=O)N[C@H](C=O)C[C@@H]1CCNC1=O] |
| A90 | 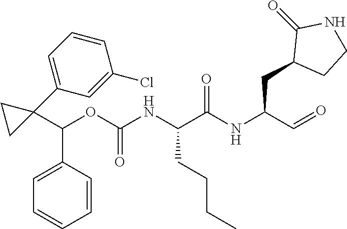 [SMILES: CCCC[C@H](NC(=O)OC(c1ccccc1)C1(c2cccc(Cl)c2)CC1)C(=O)N[C@H](C=O)C[C@@H]1CCNC1=O] |
| A91 | 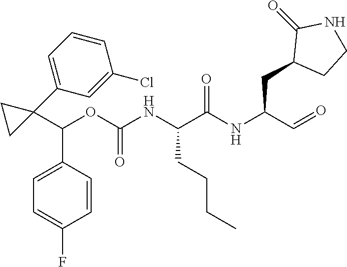 [SMILES: CCCC[C@H](NC(=O)OC(c1ccc(F)cc1)C1(c2cccc(Cl)c2)CC1)C(=O)N[C@H](C=O)C[C@@H]1CCNC1=O] |
| A92 | 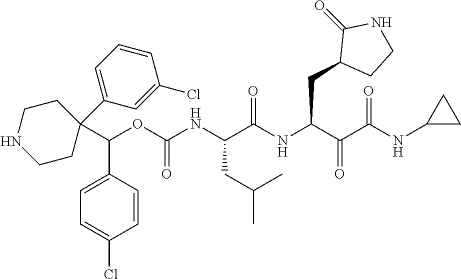 [SMILES: CC(C)C[C@H](NC(=O)OC(c1ccc(Cl)cc1)C1(c2cccc(Cl)c2)CCNCC1)C(=O)N[C@@H](C[C@@H]1CCNC1=O)C(=O)C(=O)NC1CC1] |
| A93 | 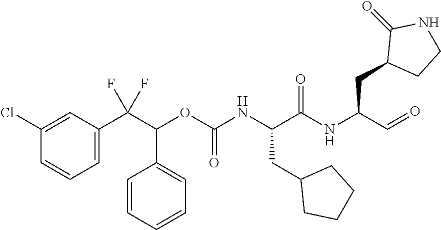 [SMILES: O=C[C@H](C[C@@H]1CCNC1=O)NC(=O)[C@H](CC1CCCC1)NC(=O)OC(c1ccccc1)C(F)(F)c1cccc(Cl)c1] |
| A94 | 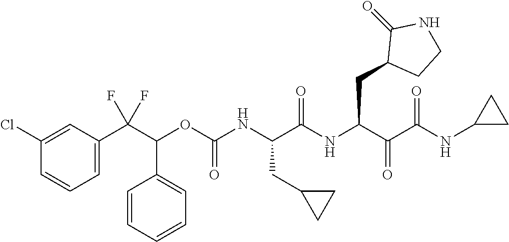 [SMILES: O=C(N[C@@H](CC1CC1)C(=O)N[C@@H](C[C@@H]1CCNC1=O)C(=O)C(=O)NC1CC1)OC(c1ccccc1)C(F)(F)c1cccc(Cl)c1] |
| A95 | 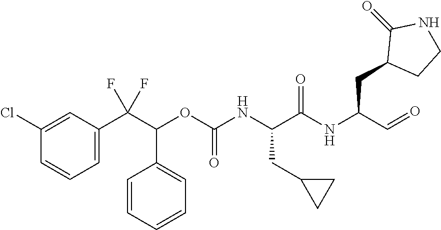 [SMILES: O=C[C@H](C[C@@H]1CCNC1=O)NC(=O)[C@H](CC1CC1)NC(=O)OC(c1ccccc1)C(F)(F)c1cccc(Cl)c1] |
| A96 | 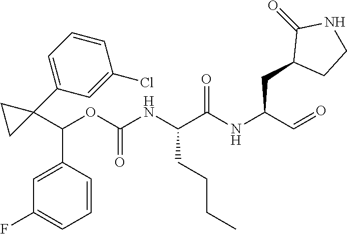 [SMILES: CCCC[C@H](NC(=O)OC(c1cccc(F)c1)C1(c2cccc(Cl)c2)CC1)C(=O)N[C@H](C=O)C[C@@H]1CCNC1=O] |
| A97 | 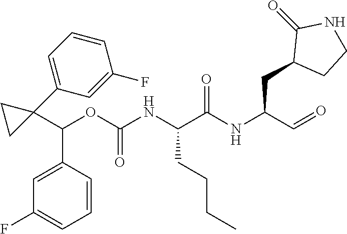 [SMILES: CCCC[C@H](NC(=O)OC(c1cccc(F)c1)C1(c2cccc(F)c2)CC1)C(=O)N[C@H](C=O)C[C@@H]1CCNC1=O] |
| A98 | 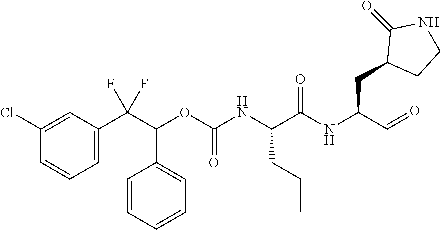 [SMILES: CCC[C@H](NC(=O)OC(c1ccccc1)C(F)(F)c1cccc(Cl)c1)C(=O)N[C@H](C=O)C[C@@H]1CCNC1=O] |
| A99 | 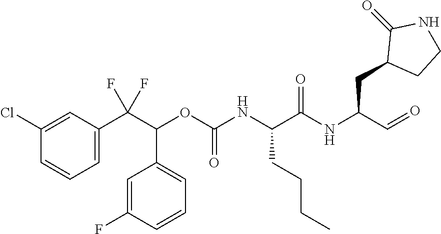 [SMILES: CCCC[C@H](NC(=O)OC(c1cccc(F)c1)C(F)(F)c1cccc(Cl)c1)C(=O)N[C@H](C=O)C[C@@H]1CCNC1=O] |
| A100 | 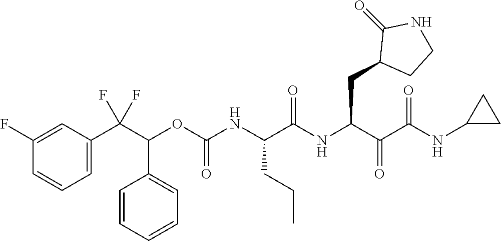 [SMILES: CCC[C@H](NC(=O)OC(c1ccccc1)C(F)(F)c1cccc(F)c1)C(=O)N[C@@H](C[C@@H]1CCNC1=O)C(=O)C(=O)NC1CC1] |
| A101 | 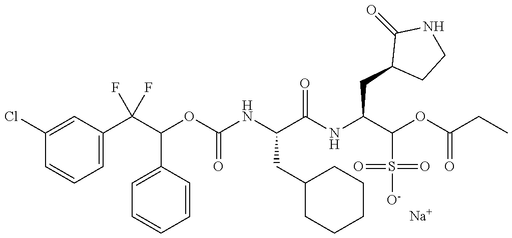 [SMILES: CCC(=O)OC([C@H](C[C@@H]1CCNC1=O)NC(=O)[C@H](CC1CCCCC1)NC(=O)OC(c1ccccc1)C(F)(F)c1cccc(Cl)c1)S(=O)(=O)[O-] ; [Na+]] |
| A102 | 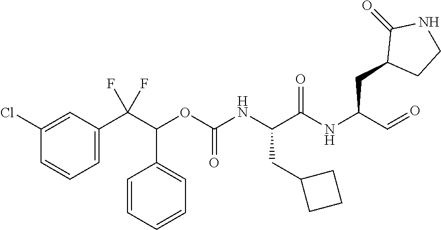 [SMILES: O=C[C@H](C[C@@H]1CCNC1=O)NC(=O)[C@H](CC1CCC1)NC(=O)OC(c1ccccc1)C(F)(F)c1cccc(Cl)c1] |
| A103 | 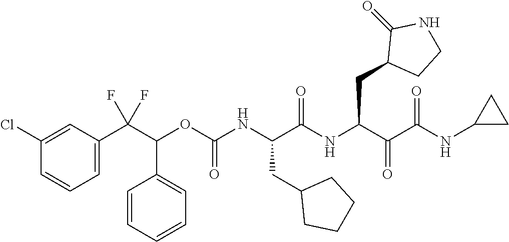 [SMILES: O=C(N[C@@H](CC1CCCC1)C(=O)N[C@@H](C[C@@H]1CCNC1=O)C(=O)C(=O)NC1CC1)OC(c1ccccc1)C(F)(F)c1cccc(Cl)c1] |
| A104 | 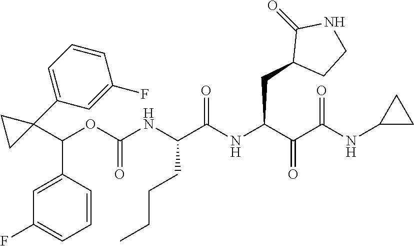 [SMILES: CCCC[C@H](NC(=O)OC(c1cccc(F)c1)C1(c2cccc(F)c2)CC1)C(=O)N[C@@H](C[C@@H]1CCNC1=O)C(=O)C(=O)NC1CC1] |
| A105 | 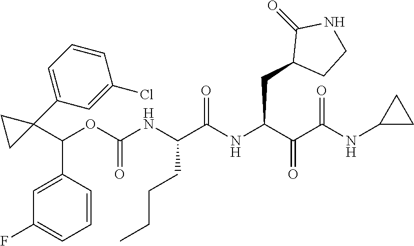 [SMILES: CCCC[C@H](NC(=O)OC(c1cccc(F)c1)C1(c2cccc(Cl)c2)CC1)C(=O)N[C@@H](C[C@@H]1CCNC1=O)C(=O)C(=O)NC1CC1] |
| A106 | 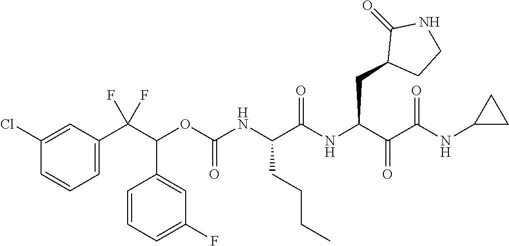 [SMILES: CCCC[C@H](NC(=O)OC(c1cccc(F)c1)C(F)(F)c1cccc(Cl)c1)C(=O)N[C@@H](C[C@@H]1CCNC1=O)C(=O)C(=O)NC1CC1] |
| A107 | 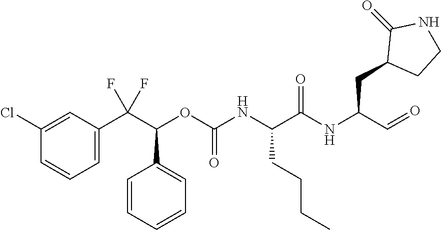 [SMILES: CCCC[C@H](NC(=O)O[C@@H](c1ccccc1)C(F)(F)c1cccc(Cl)c1)C(=O)N[C@H](C=O)C[C@@H]1CCNC1=O] |
| A108 | 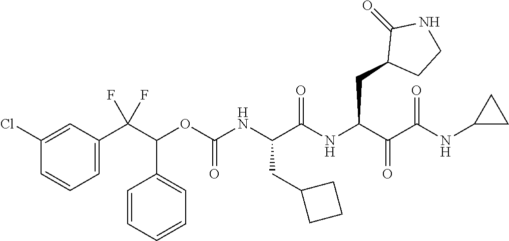 [SMILES: O=C(N[C@@H](CC1CCC1)C(=O)N[C@@H](C[C@@H]1CCNC1=O)C(=O)C(=O)NC1CC1)OC(c1ccccc1)C(F)(F)c1cccc(Cl)c1] |
| A109 | 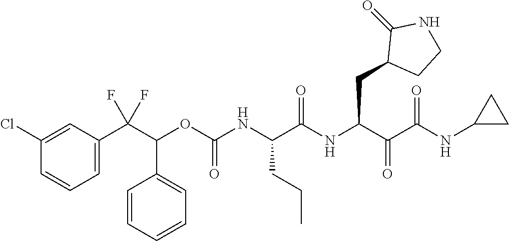 [SMILES: CCC[C@H](NC(=O)OC(c1ccccc1)C(F)(F)c1cccc(Cl)c1)C(=O)N[C@@H](C[C@@H]1CCNC1=O)C(=O)C(=O)NC1CC1] |
| A110 | 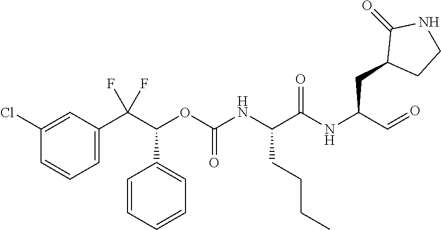 [SMILES: CCCC[C@H](NC(=O)O[C@H](c1ccccc1)C(F)(F)c1cccc(Cl)c1)C(=O)N[C@H](C=O)C[C@@H]1CCNC1=O] |
| A111 | 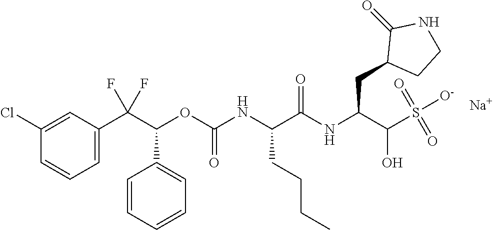 [SMILES: CCCC[C@H](NC(=O)O[C@H](c1ccccc1)C(F)(F)c1cccc(Cl)c1)C(=O)N[C@@H](C[C@@H]1CCNC1=O)C(O)S(=O)(=O)[O-] ; [Na+]] |
| A112 | 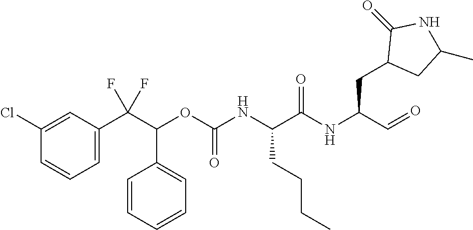 [SMILES: CCCC[C@H](NC(=O)OC(c1ccccc1)C(F)(F)c1cccc(Cl)c1)C(=O)N[C@H](C=O)CC1CC(C)NC1=O] |
| A113 | 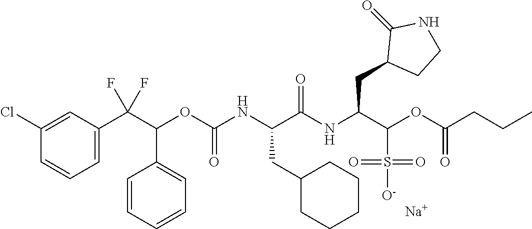 [SMILES: CCCC(=O)OC([C@H](C[C@@H]1CCNC1=O)NC(=O)[C@H](CC1CCCCC1)NC(=O)OC(c1ccccc1)C(F)(F)c1cccc(Cl)c1)S(=O)(=O)[O-] ; [Na+]] |
| A114 | 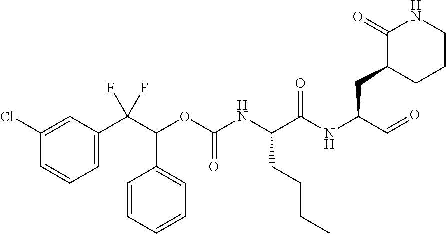 [SMILES: CCCC[C@H](NC(=O)OC(c1ccccc1)C(F)(F)c1cccc(Cl)c1)C(=O)N[C@H](C=O)C[C@@H]1CCCNC1=O] |
| A115 | 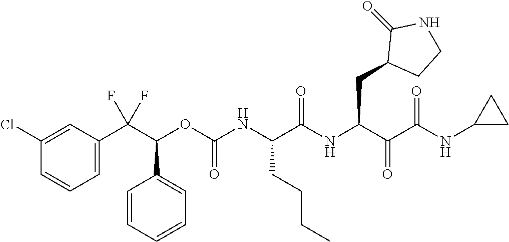 [SMILES: CCCC[C@H](NC(=O)O[C@@H](c1ccccc1)C(F)(F)c1cccc(Cl)c1)C(=O)N[C@@H](C[C@@H]1CCNC1=O)C(=O)C(=O)NC1CC1] |
| A116 | 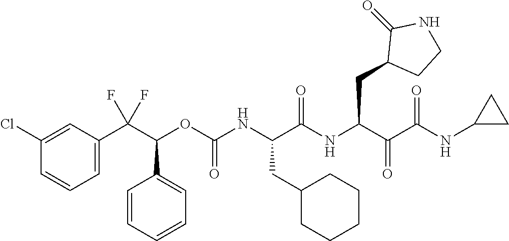 [SMILES: O=C(N[C@@H](CC1CCCCC1)C(=O)N[C@@H](C[C@@H]1CCNC1=O)C(=O)C(=O)NC1CC1)O[C@@H](c1ccccc1)C(F)(F)c1cccc(Cl)c1] |
| A117 | 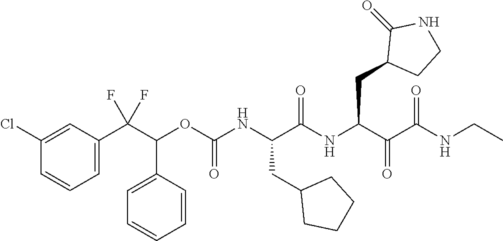 [SMILES: CCNC(=O)C(=O)[C@H](C[C@@H]1CCNC1=O)NC(=O)[C@H](CC1CCCC1)NC(=O)OC(c1ccccc1)C(F)(F)c1cccc(Cl)c1] |
| A119 | 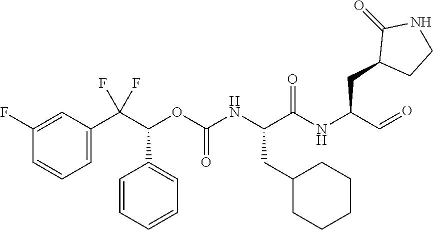 [SMILES: O=C[C@H](C[C@@H]1CCNC1=O)NC(=O)[C@H](CC1CCCCC1)NC(=O)O[C@H](c1ccccc1)C(F)(F)c1cccc(F)c1] |
| A120 | 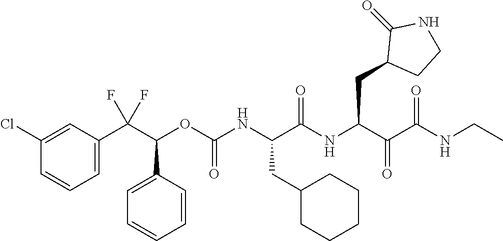 [SMILES: CCNC(=O)C(=O)[C@H](C[C@@H]1CCNC1=O)NC(=O)[C@H](CC1CCCCC1)NC(=O)O[C@@H](c1ccccc1)C(F)(F)c1cccc(Cl)c1] |
| A121 | 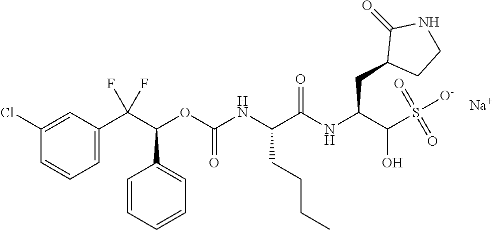 [SMILES: CCCC[C@H](NC(=O)O[C@@H](c1ccccc1)C(F)(F)c1cccc(Cl)c1)C(=O)N[C@@H](C[C@@H]1CCNC1=O)C(O)S(=O)(=O)[O-] ; [Na+]] |
| A122 | 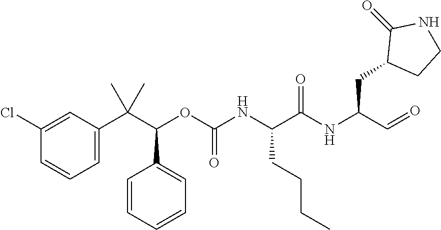 [SMILES: CCCC[C@H](NC(=O)O[C@@H](c1ccccc1)C(C)(C)c1cccc(Cl)c1)C(=O)N[C@H](C=O)C[C@H]1CCNC1=O] |
| A123 | 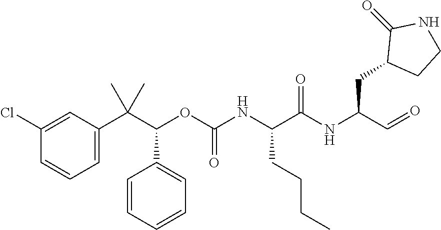 [SMILES: CCCC[C@H](NC(=O)O[C@H](c1ccccc1)C(C)(C)c1cccc(Cl)c1)C(=O)N[C@H](C=O)C[C@H]1CCNC1=O] |
| A124 | 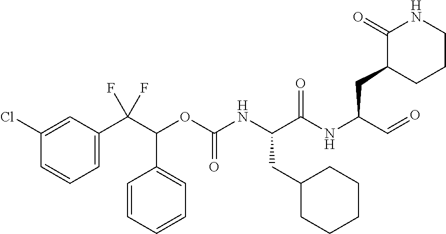 [SMILES: O=C[C@H](C[C@@H]1CCCNC1=O)NC(=O)[C@H](CC1CCCCC1)NC(=O)OC(c1ccccc1)C(F)(F)c1cccc(Cl)c1] |
| A125 | 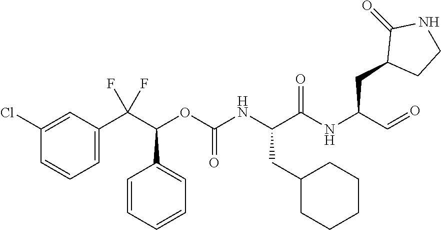 [SMILES: O=C[C@H](C[C@@H]1CCNC1=O)NC(=O)[C@H](CC1CCCCC1)NC(=O)O[C@@H](c1ccccc1)C(F)(F)c1cccc(Cl)c1] |
| A126 | 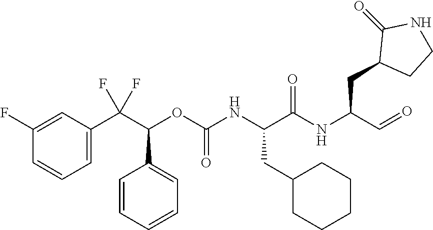 [SMILES: O=C[C@H](C[C@@H]1CCNC1=O)NC(=O)[C@H](CC1CCCCC1)NC(=O)O[C@@H](c1ccccc1)C(F)(F)c1cccc(F)c1] |
| A127 | 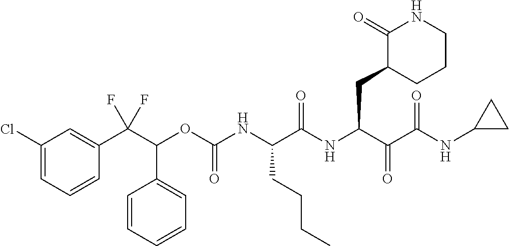 [SMILES: CCCC[C@H](NC(=O)OC(c1ccccc1)C(F)(F)c1cccc(Cl)c1)C(=O)N[C@@H](C[C@@H]1CCCNC1=O)C(=O)C(=O)NC1CC1] |
| A128 | 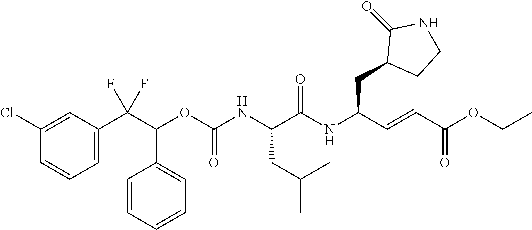 [SMILES: CCOC(=O)/C=C/[C@H](C[C@@H]1CCNC1=O)NC(=O)[C@H](CC(C)C)NC(=O)OC(c1ccccc1)C(F)(F)c1cccc(Cl)c1] |
| A129 | 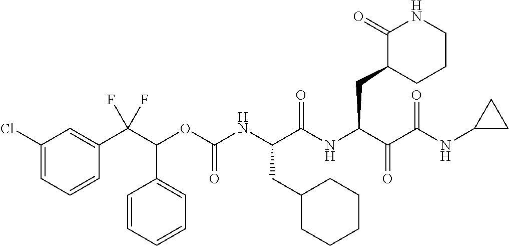 [SMILES: O=C(N[C@@H](CC1CCCCC1)C(=O)N[C@@H](C[C@@H]1CCCNC1=O)C(=O)C(=O)NC1CC1)OC(c1ccccc1)C(F)(F)c1cccc(Cl)c1] |
| A130 | 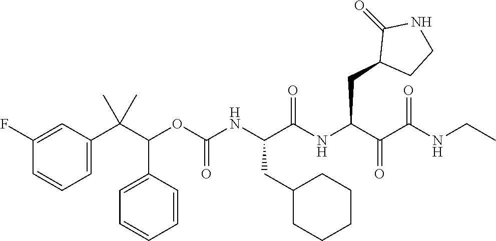 [SMILES: CCNC(=O)C(=O)[C@H](C[C@@H]1CCNC1=O)NC(=O)[C@H](CC1CCCCC1)NC(=O)OC(c1ccccc1)C(C)(C)c1cccc(F)c1] |
| A131 | 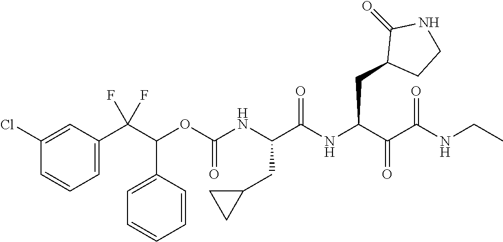 [SMILES: CCNC(=O)C(=O)[C@H](C[C@@H]1CCNC1=O)NC(=O)[C@H](CC1CC1)NC(=O)OC(c1ccccc1)C(F)(F)c1cccc(Cl)c1] |
| A132 | 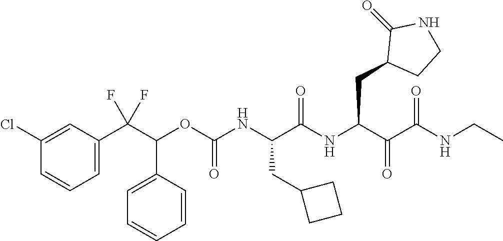 [SMILES: CCNC(=O)C(=O)[C@H](C[C@@H]1CCNC1=O)NC(=O)[C@H](CC1CCC1)NC(=O)OC(c1ccccc1)C(F)(F)c1cccc(Cl)c1] |
| A133 | 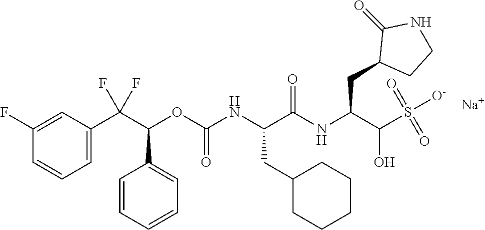 [SMILES: O=C(N[C@@H](CC1CCCCC1)C(=O)N[C@@H](C[C@@H]1CCNC1=O)C(O)S(=O)(=O)[O-])O[C@@H](c1ccccc1)C(F)(F)c1cccc(F)c1 ; [Na+]] |
| A134 | 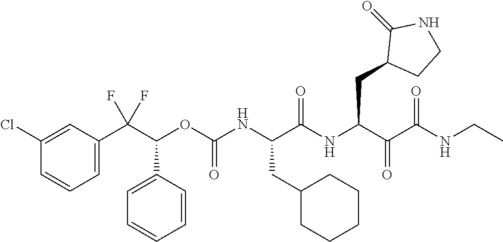 [SMILES: CCNC(=O)C(=O)[C@H](C[C@@H]1CCNC1=O)NC(=O)[C@H](CC1CCCCC1)NC(=O)O[C@H](c1ccccc1)C(F)(F)c1cccc(Cl)c1] |
| A135 | 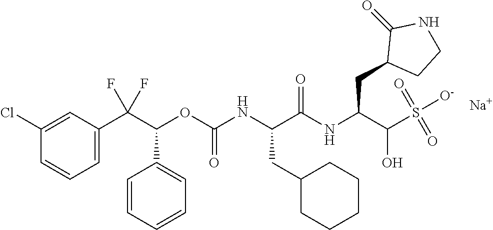 [SMILES: O=C(N[C@@H](CC1CCCCC1)C(=O)N[C@@H](C[C@@H]1CCNC1=O)C(O)S(=O)(=O)[O-])O[C@H](c1ccccc1)C(F)(F)c1cccc(Cl)c1 ; [Na+]] |
| A136 | 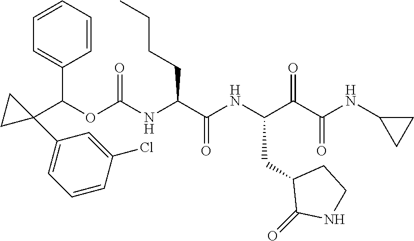 [SMILES: CCCC[C@H](NC(=O)OC(c1ccccc1)C1(c2cccc(Cl)c2)CC1)C(=O)N[C@@H](C[C@@H]1CCNC1=O)C(=O)C(=O)NC1CC1] |
| A137 | 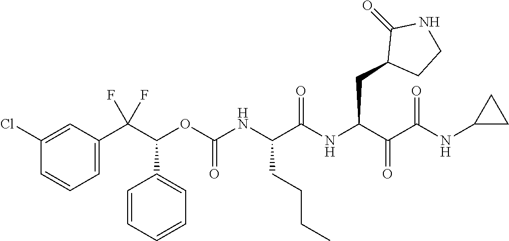 [SMILES: CCCC[C@H](NC(=O)O[C@H](c1ccccc1)C(F)(F)c1cccc(Cl)c1)C(=O)N[C@@H](C[C@@H]1CCNC1=O)C(=O)C(=O)NC1CC1] |
| A138 | 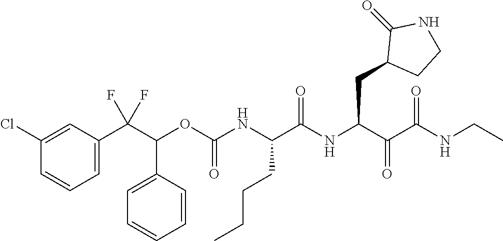 [SMILES: CCCC[C@H](NC(=O)OC(c1ccccc1)C(F)(F)c1cccc(Cl)c1)C(=O)N[C@@H](C[C@@H]1CCNC1=O)C(=O)C(=O)NCC] |
| A139 | 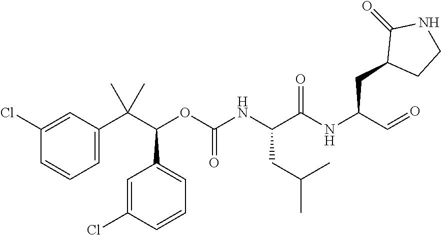 [SMILES: CC(C)C[C@H](NC(=O)O[C@@H](c1cccc(Cl)c1)C(C)(C)c1cccc(Cl)c1)C(=O)N[C@H](C=O)C[C@@H]1CCNC1=O] |
| A140 | 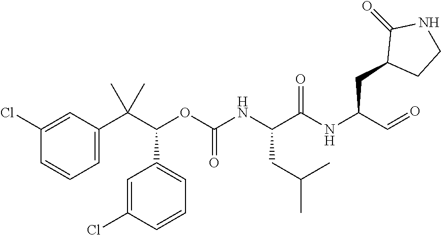 [SMILES: CC(C)C[C@H](NC(=O)O[C@H](c1cccc(Cl)c1)C(C)(C)c1cccc(Cl)c1)C(=O)N[C@H](C=O)C[C@@H]1CCNC1=O] |
| A141 | 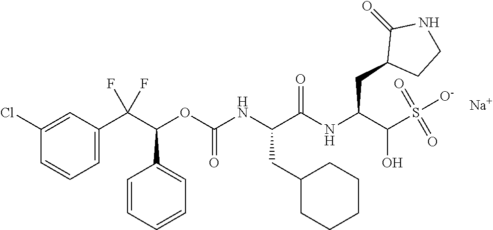 [SMILES: O=C(N[C@@H](CC1CCCCC1)C(=O)N[C@@H](C[C@@H]1CCNC1=O)C(O)S(=O)(=O)[O-])O[C@@H](c1ccccc1)C(F)(F)c1cccc(Cl)c1 ; [Na+]] |
| A142 | 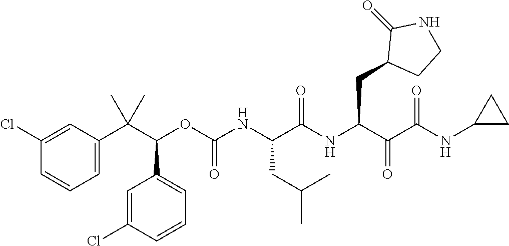 [SMILES: CC(C)C[C@H](NC(=O)O[C@@H](c1cccc(Cl)c1)C(C)(C)c1cccc(Cl)c1)C(=O)N[C@@H](C[C@@H]1CCNC1=O)C(=O)C(=O)NC1CC1] |
| A143 | 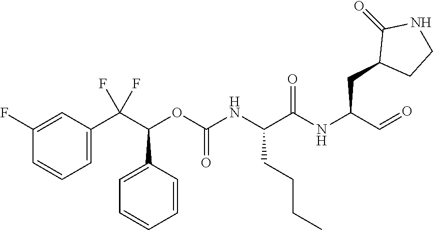 [SMILES: CCCC[C@H](NC(=O)O[C@@H](c1ccccc1)C(F)(F)c1cccc(F)c1)C(=O)N[C@H](C=O)C[C@@H]1CCNC1=O] |
| A144 | 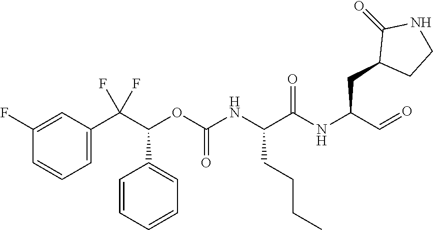 [SMILES: CCCC[C@H](NC(=O)O[C@H](c1ccccc1)C(F)(F)c1cccc(F)c1)C(=O)N[C@H](C=O)C[C@@H]1CCNC1=O] |
| A145 | 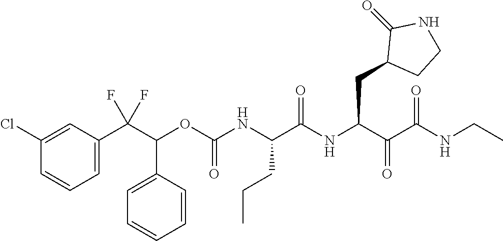 [SMILES: CCC[C@H](NC(=O)OC(c1ccccc1)C(F)(F)c1cccc(Cl)c1)C(=O)N[C@@H](C[C@@H]1CCNC1=O)C(=O)C(=O)NCC] |
| A146 | 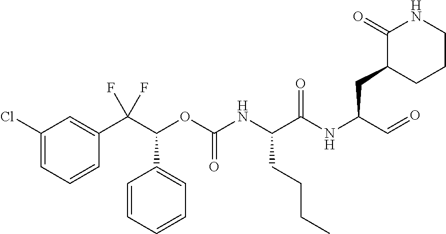 [SMILES: CCCC[C@H](NC(=O)O[C@H](c1ccccc1)C(F)(F)c1cccc(Cl)c1)C(=O)N[C@H](C=O)C[C@@H]1CCCNC1=O] |
| A147 | 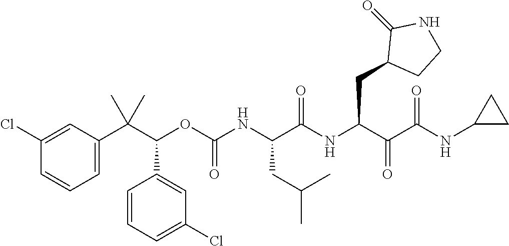 [SMILES: CC(C)C[C@H](NC(=O)O[C@H](c1cccc(Cl)c1)C(C)(C)c1cccc(Cl)c1)C(=O)N[C@@H](C[C@@H]1CCNC1=O)C(=O)C(=O)NC1CC1] |
| A148 | 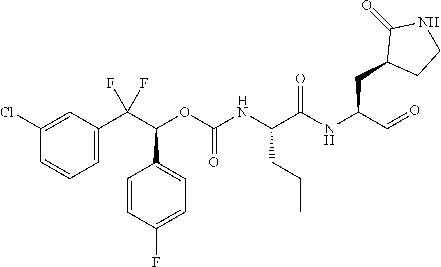 [SMILES: CCC[C@H](NC(=O)O[C@@H](c1ccc(F)cc1)C(F)(F)c1cccc(Cl)c1)C(=O)N[C@H](C=O)C[C@@H]1CCNC1=O] |
| A149 | 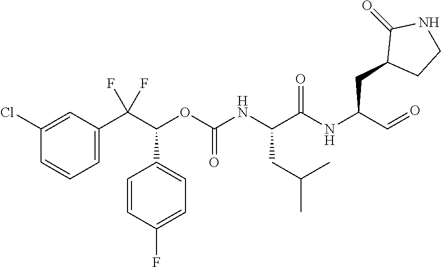 [SMILES: CC(C)C[C@H](NC(=O)O[C@H](c1ccc(F)cc1)C(F)(F)c1cccc(Cl)c1)C(=O)N[C@H](C=O)C[C@@H]1CCNC1=O] |
| A151 | 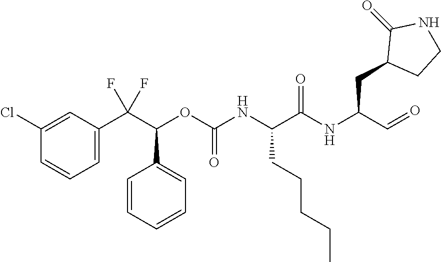 [SMILES: CCCCC[C@H](NC(=O)O[C@@H](c1ccccc1)C(F)(F)c1cccc(Cl)c1)C(=O)N[C@H](C=O)C[C@@H]1CCNC1=O] |
| A152 | 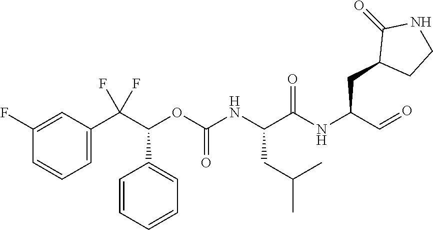 [SMILES: CC(C)C[C@H](NC(=O)O[C@H](c1ccccc1)C(F)(F)c1cccc(F)c1)C(=O)N[C@H](C=O)C[C@@H]1CCNC1=O] |
| A153 | 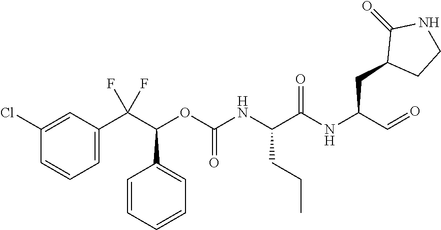 [SMILES: CCC[C@H](NC(=O)O[C@@H](c1ccccc1)C(F)(F)c1cccc(Cl)c1)C(=O)N[C@H](C=O)C[C@@H]1CCNC1=O] |
| A154 | 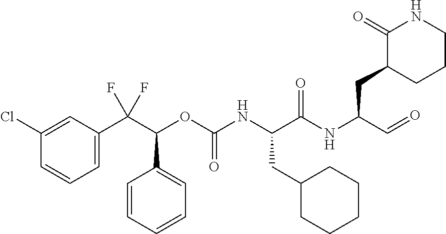 [SMILES: O=C[C@H](C[C@@H]1CCCNC1=O)NC(=O)[C@H](CC1CCCCC1)NC(=O)O[C@@H](c1ccccc1)C(F)(F)c1cccc(Cl)c1] |
| A155 | 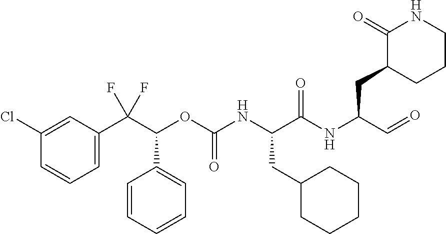 [SMILES: O=C[C@H](C[C@@H]1CCCNC1=O)NC(=O)[C@H](CC1CCCCC1)NC(=O)O[C@H](c1ccccc1)C(F)(F)c1cccc(Cl)c1] |
| A156 | 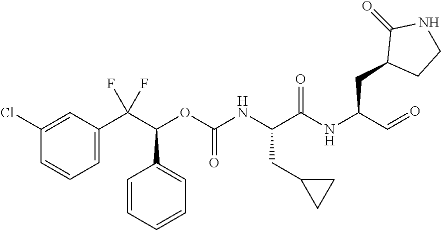 [SMILES: O=C[C@H](C[C@@H]1CCNC1=O)NC(=O)[C@H](CC1CC1)NC(=O)O[C@@H](c1ccccc1)C(F)(F)c1cccc(Cl)c1] |
| A157 | 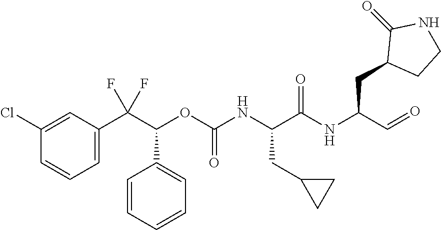 [SMILES: O=C[C@H](C[C@@H]1CCNC1=O)NC(=O)[C@H](CC1CC1)NC(=O)O[C@H](c1ccccc1)C(F)(F)c1cccc(Cl)c1] |
| A159 | 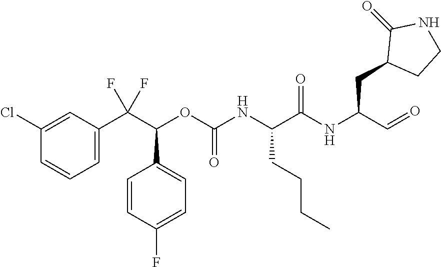 [SMILES: CCCC[C@H](NC(=O)O[C@@H](c1ccc(F)cc1)C(F)(F)c1cccc(Cl)c1)C(=O)N[C@H](C=O)C[C@@H]1CCNC1=O] |
| A160 | 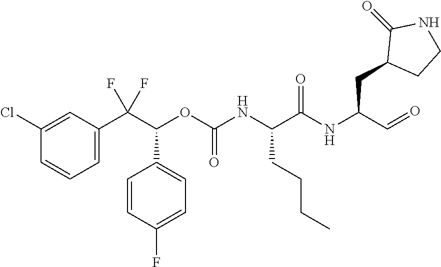 [SMILES: CCCC[C@H](NC(=O)O[C@H](c1ccc(F)cc1)C(F)(F)c1cccc(Cl)c1)C(=O)N[C@H](C=O)C[C@@H]1CCNC1=O] |
| A161 | 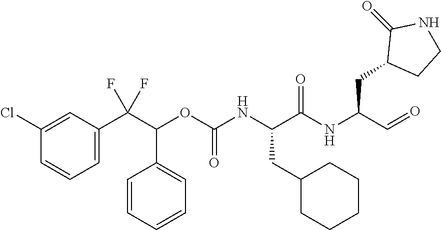 [SMILES: O=C[C@H](C[C@H]1CCNC1=O)NC(=O)[C@H](CC1CCCCC1)NC(=O)OC(c1ccccc1)C(F)(F)c1cccc(Cl)c1] |
| A162 | 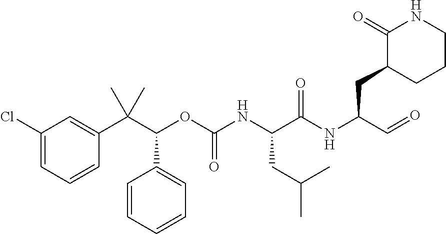 [SMILES: CC(C)C[C@H](NC(=O)O[C@H](c1ccccc1)C(C)(C)c1cccc(Cl)c1)C(=O)N[C@H](C=O)C[C@@H]1CCCNC1=O] |
| A163 | 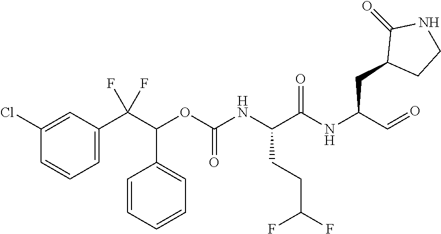 [SMILES: O=C[C@H](C[C@@H]1CCNC1=O)NC(=O)[C@H](CCC(F)F)NC(=O)OC(c1ccccc1)C(F)(F)c1cccc(Cl)c1] |
| A164 | 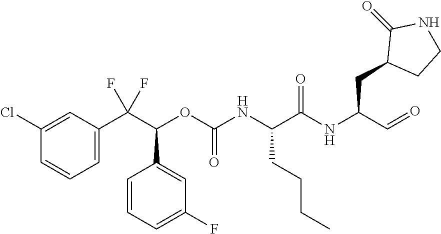 [SMILES: CCCC[C@H](NC(=O)O[C@@H](c1cccc(F)c1)C(F)(F)c1cccc(Cl)c1)C(=O)N[C@H](C=O)C[C@@H]1CCNC1=O] |
| A165 | 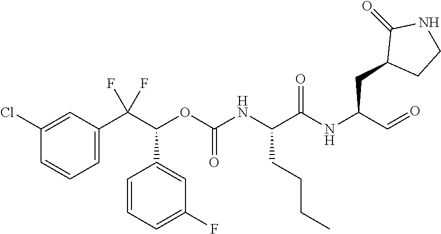 [SMILES: CCCC[C@H](NC(=O)O[C@H](c1cccc(F)c1)C(F)(F)c1cccc(Cl)c1)C(=O)N[C@H](C=O)C[C@@H]1CCNC1=O] |
| A166 | 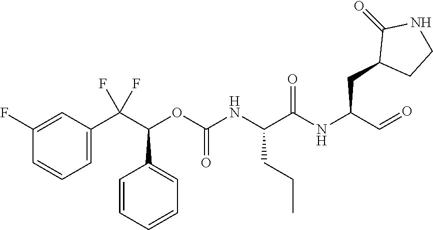 [SMILES: CCC[C@H](NC(=O)O[C@@H](c1ccccc1)C(F)(F)c1cccc(F)c1)C(=O)N[C@H](C=O)C[C@@H]1CCNC1=O] |
| A167 | 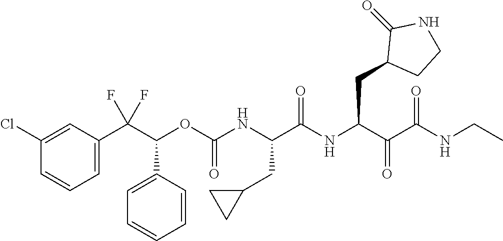 [SMILES: CCNC(=O)C(=O)[C@H](C[C@@H]1CCNC1=O)NC(=O)[C@H](CC1CC1)NC(=O)O[C@H](c1ccccc1)C(F)(F)c1cccc(Cl)c1] |
| A168 | 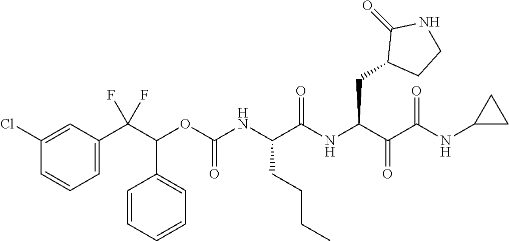 [SMILES: CCCC[C@H](NC(=O)OC(c1ccccc1)C(F)(F)c1cccc(Cl)c1)C(=O)N[C@@H](C[C@H]1CCNC1=O)C(=O)C(=O)NC1CC1] |
| A169 | 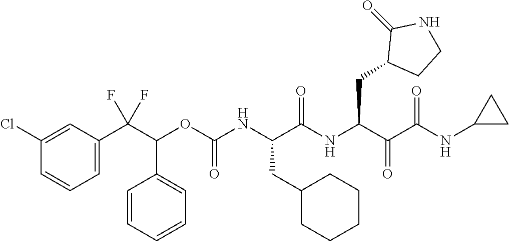 [SMILES: O=C(N[C@@H](CC1CCCCC1)C(=O)N[C@@H](C[C@H]1CCNC1=O)C(=O)C(=O)NC1CC1)OC(c1ccccc1)C(F)(F)c1cccc(Cl)c1] |
| A170 | 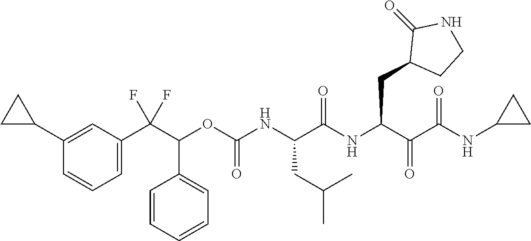 [SMILES: CC(C)C[C@H](NC(=O)OC(c1ccccc1)C(F)(F)c1cccc(C2CC2)c1)C(=O)N[C@@H](C[C@@H]1CCNC1=O)C(=O)C(=O)NC1CC1] |
| A171 | 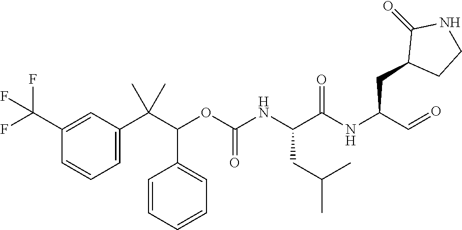 [SMILES: CC(C)C[C@H](NC(=O)OC(c1ccccc1)C(C)(C)c1cccc(C(F)(F)F)c1)C(=O)N[C@H](C=O)C[C@@H]1CCNC1=O] |
| A172 | 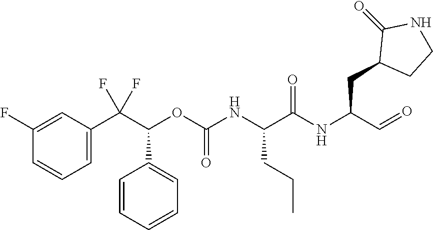 [SMILES: CCC[C@H](NC(=O)O[C@H](c1ccccc1)C(F)(F)c1cccc(F)c1)C(=O)N[C@H](C=O)C[C@@H]1CCNC1=O] |
| A173 | 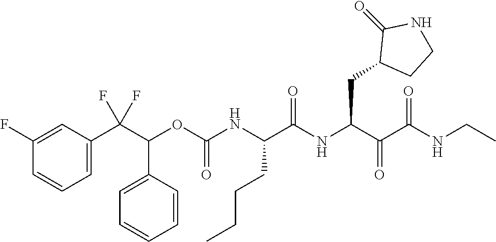 [SMILES: CCCC[C@H](NC(=O)OC(c1ccccc1)C(F)(F)c1cccc(F)c1)C(=O)N[C@@H](C[C@H]1CCNC1=O)C(=O)C(=O)NCC] |
| A174 | 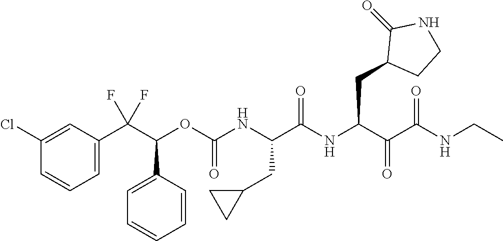 [SMILES: CCNC(=O)C(=O)[C@H](C[C@@H]1CCNC1=O)NC(=O)[C@H](CC1CC1)NC(=O)O[C@@H](c1ccccc1)C(F)(F)c1cccc(Cl)c1] |
| A175 | 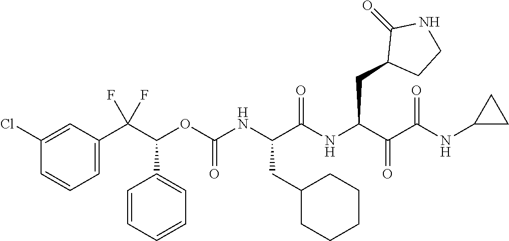 [SMILES: O=C(N[C@@H](CC1CCCCC1)C(=O)N[C@@H](C[C@@H]1CCNC1=O)C(=O)C(=O)NC1CC1)O[C@H](c1ccccc1)C(F)(F)c1cccc(Cl)c1] |
| A176 | 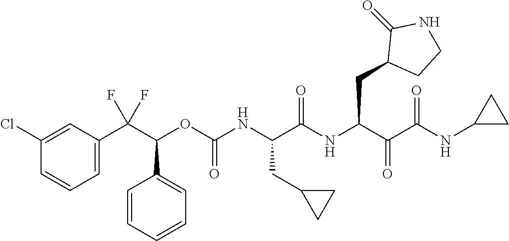 [SMILES: O=C(N[C@@H](CC1CC1)C(=O)N[C@@H](C[C@@H]1CCNC1=O)C(=O)C(=O)NC1CC1)O[C@@H](c1ccccc1)C(F)(F)c1cccc(Cl)c1] |
| A177 | 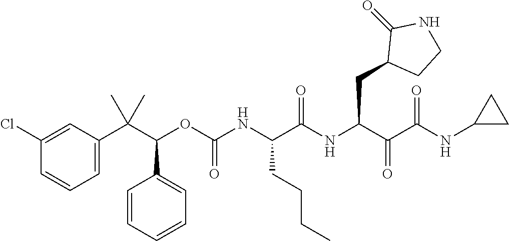 [SMILES: CCCC[C@H](NC(=O)O[C@@H](c1ccccc1)C(C)(C)c1cccc(Cl)c1)C(=O)N[C@@H](C[C@@H]1CCNC1=O)C(=O)C(=O)NC1CC1] |
| A178 | 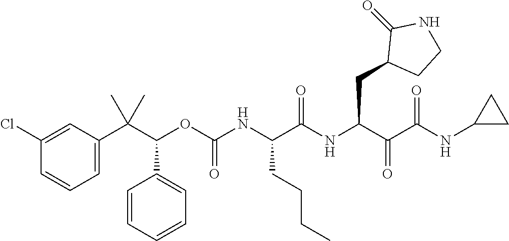 [SMILES: CCCC[C@H](NC(=O)O[C@H](c1ccccc1)C(C)(C)c1cccc(Cl)c1)C(=O)N[C@@H](C[C@@H]1CCNC1=O)C(=O)C(=O)NC1CC1] |
| A179 | 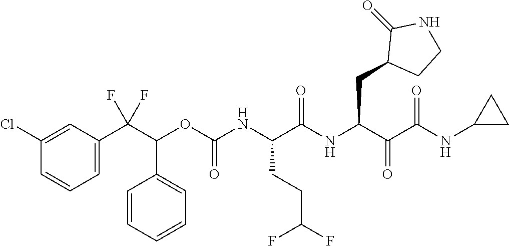 [SMILES: O=C(N[C@@H](CCC(F)F)C(=O)N[C@@H](C[C@@H]1CCNC1=O)C(=O)C(=O)NC1CC1)OC(c1ccccc1)C(F)(F)c1cccc(Cl)c1] |
| A180 | 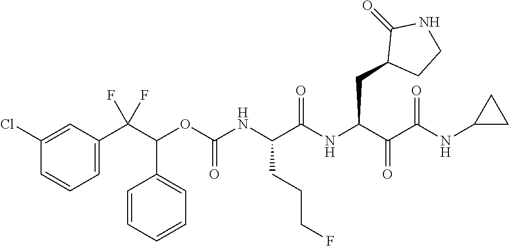 [SMILES: O=C(N[C@@H](CCCF)C(=O)N[C@@H](C[C@@H]1CCNC1=O)C(=O)C(=O)NC1CC1)OC(c1ccccc1)C(F)(F)c1cccc(Cl)c1] |
| A181 | 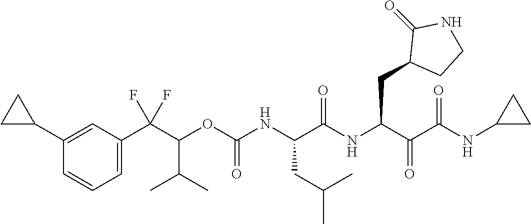 [SMILES: CC(C)C[C@H](NC(=O)OC(C(C)C)C(F)(F)c1cccc(C2CC2)c1)C(=O)N[C@@H](C[C@@H]1CCNC1=O)C(=O)C(=O)NC1CC1] |
| A182 | 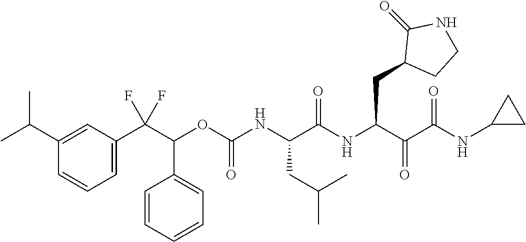 [SMILES: CC(C)C[C@H](NC(=O)OC(c1ccccc1)C(F)(F)c1cccc(C(C)C)c1)C(=O)N[C@@H](C[C@@H]1CCNC1=O)C(=O)C(=O)NC1CC1] |
| A183 | 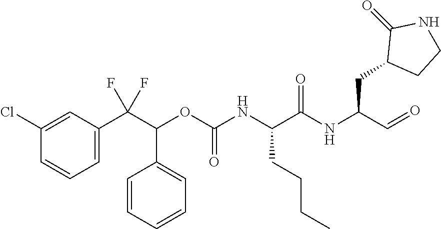 [SMILES: CCCC[C@H](NC(=O)OC(c1ccccc1)C(F)(F)c1cccc(Cl)c1)C(=O)N[C@H](C=O)C[C@H]1CCNC1=O] |
| A185 | 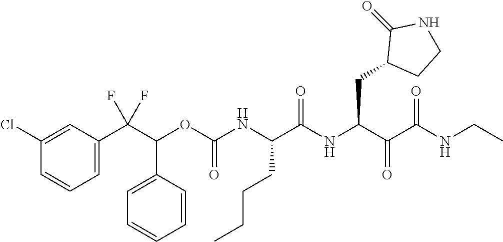 [SMILES: CCCC[C@H](NC(=O)OC(c1ccccc1)C(F)(F)c1cccc(Cl)c1)C(=O)N[C@@H](C[C@H]1CCNC1=O)C(=O)C(=O)NCC] |
| A186 | 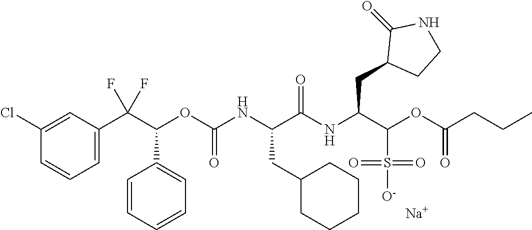 [SMILES: CCCC(=O)OC([C@H](C[C@@H]1CCNC1=O)NC(=O)[C@H](CC1CCCCC1)NC(=O)O[C@H](c1ccccc1)C(F)(F)c1cccc(Cl)c1)S(=O)(=O)[O-] ; [Na+]] |
| A187 | 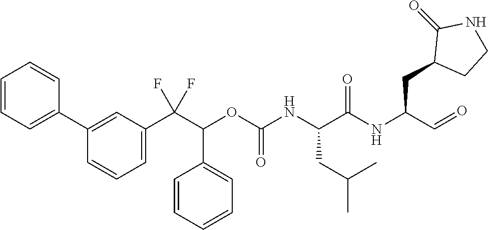 [SMILES: CC(C)C[C@H](NC(=O)OC(c1ccccc1)C(F)(F)c1cccc(-c2ccccc2)c1)C(=O)N[C@H](C=O)C[C@@H]1CCNC1=O] |
| A188 | 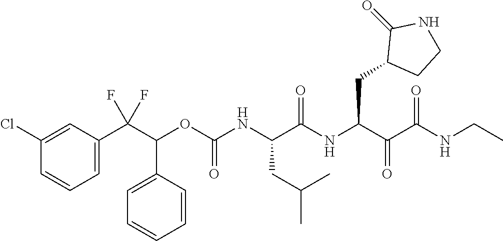 [SMILES: CCNC(=O)C(=O)[C@H](C[C@H]1CCNC1=O)NC(=O)[C@H](CC(C)C)NC(=O)OC(c1ccccc1)C(F)(F)c1cccc(Cl)c1] |
| A189 | 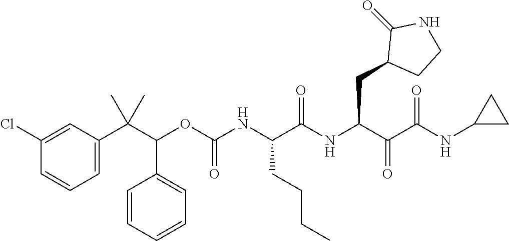 [SMILES: CCCC[C@H](NC(=O)OC(c1ccccc1)C(C)(C)c1cccc(Cl)c1)C(=O)N[C@@H](C[C@@H]1CCNC1=O)C(=O)C(=O)NC1CC1] |
| A191 | 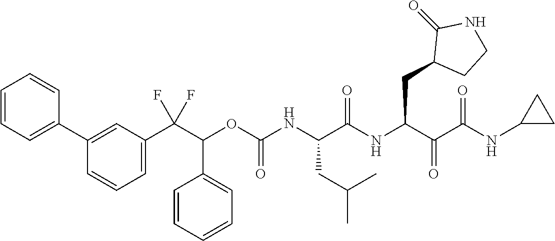 [SMILES: CC(C)C[C@H](NC(=O)OC(c1ccccc1)C(F)(F)c1cccc(-c2ccccc2)c1)C(=O)N[C@@H](C[C@@H]1CCNC1=O)C(=O)C(=O)NC1CC1] |
| A192 | 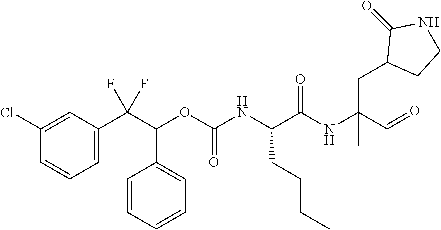 [SMILES: CCCC[C@H](NC(=O)OC(c1ccccc1)C(F)(F)c1cccc(Cl)c1)C(=O)NC(C)(C=O)CC1CCNC1=O] |
| A193 | 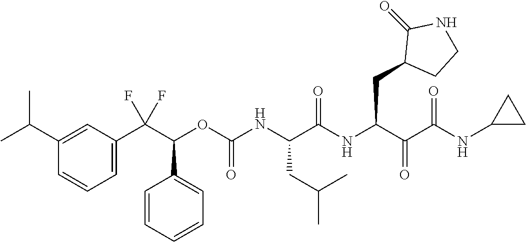 [SMILES: CC(C)C[C@H](NC(=O)O[C@@H](c1ccccc1)C(F)(F)c1cccc(C(C)C)c1)C(=O)N[C@@H](C[C@@H]1CCNC1=O)C(=O)C(=O)NC1CC1] |
| A194 | 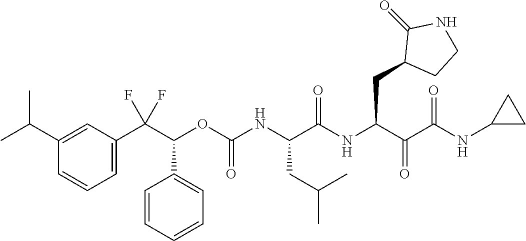 [SMILES: CC(C)C[C@H](NC(=O)O[C@H](c1ccccc1)C(F)(F)c1cccc(C(C)C)c1)C(=O)N[C@@H](C[C@@H]1CCNC1=O)C(=O)C(=O)NC1CC1] |
| A195 | 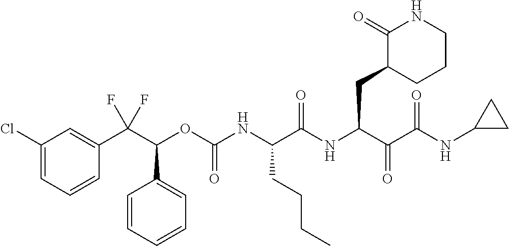 [SMILES: CCCC[C@H](NC(=O)O[C@@H](c1ccccc1)C(F)(F)c1cccc(Cl)c1)C(=O)N[C@@H](C[C@@H]1CCCNC1=O)C(=O)C(=O)NC1CC1] |
| A196 | 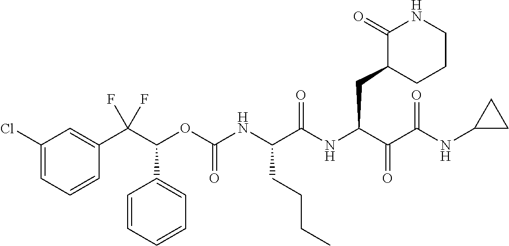 [SMILES: CCCC[C@H](NC(=O)O[C@H](c1ccccc1)C(F)(F)c1cccc(Cl)c1)C(=O)N[C@@H](C[C@@H]1CCCNC1=O)C(=O)C(=O)NC1CC1] |
| A197 | 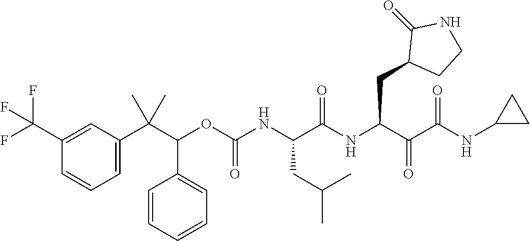 [SMILES: CC(C)C[C@H](NC(=O)OC(c1ccccc1)C(C)(C)c1cccc(C(F)(F)F)c1)C(=O)N[C@@H](C[C@@H]1CCNC1=O)C(=O)C(=O)NC1CC1] |
| A198 | 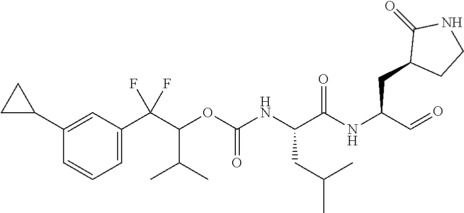 [SMILES: CC(C)C[C@H](NC(=O)OC(C(C)C)C(F)(F)c1cccc(C2CC2)c1)C(=O)N[C@H](C=O)C[C@@H]1CCNC1=O] |
| A199 | 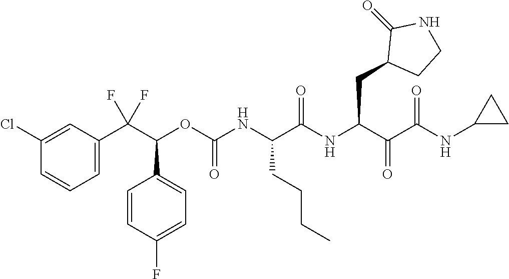 [SMILES: CCCC[C@H](NC(=O)O[C@@H](c1ccc(F)cc1)C(F)(F)c1cccc(Cl)c1)C(=O)N[C@@H](C[C@@H]1CCNC1=O)C(=O)C(=O)NC1CC1] |
| A200 | 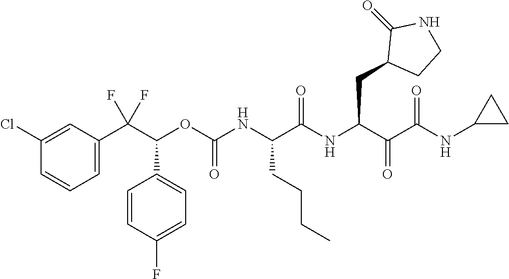 [SMILES: CCCC[C@H](NC(=O)O[C@H](c1ccc(F)cc1)C(F)(F)c1cccc(Cl)c1)C(=O)N[C@@H](C[C@@H]1CCNC1=O)C(=O)C(=O)NC1CC1] |
| A201 | 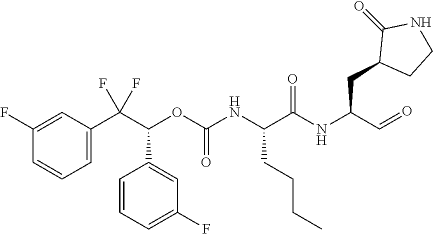 [SMILES: CCCC[C@H](NC(=O)O[C@H](c1cccc(F)c1)C(F)(F)c1cccc(F)c1)C(=O)N[C@H](C=O)C[C@@H]1CCNC1=O] |
| A202 | 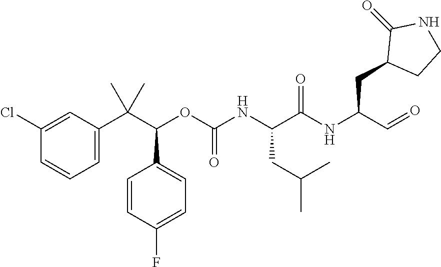 [SMILES: CC(C)C[C@H](NC(=O)O[C@@H](c1ccc(F)cc1)C(C)(C)c1cccc(Cl)c1)C(=O)N[C@H](C=O)C[C@@H]1CCNC1=O] |
| A203 | 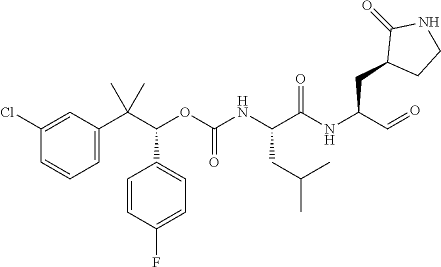 [SMILES: CC(C)C[C@H](NC(=O)O[C@H](c1ccc(F)cc1)C(C)(C)c1cccc(Cl)c1)C(=O)N[C@H](C=O)C[C@@H]1CCNC1=O] |
| A204 | 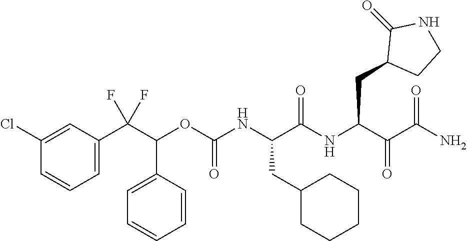 [SMILES: NC(=O)C(=O)[C@H](C[C@@H]1CCNC1=O)NC(=O)[C@H](CC1CCCCC1)NC(=O)OC(c1ccccc1)C(F)(F)c1cccc(Cl)c1] |
| A205 | 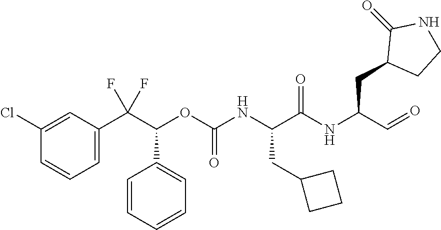 [SMILES: O=C[C@H](C[C@@H]1CCNC1=O)NC(=O)[C@H](CC1CCC1)NC(=O)O[C@H](c1ccccc1)C(F)(F)c1cccc(Cl)c1] |
| A206 | 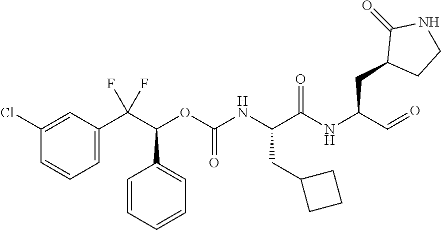 [SMILES: O=C[C@H](C[C@@H]1CCNC1=O)NC(=O)[C@H](CC1CCC1)NC(=O)O[C@@H](c1ccccc1)C(F)(F)c1cccc(Cl)c1] |
| A207 | 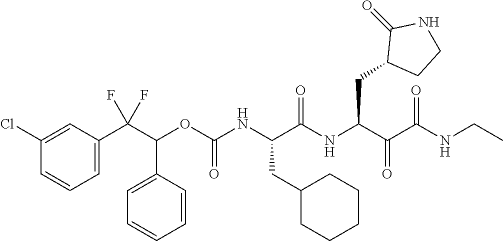 [SMILES: CCNC(=O)C(=O)[C@H](C[C@H]1CCNC1=O)NC(=O)[C@H](CC1CCCCC1)NC(=O)OC(c1ccccc1)C(F)(F)c1cccc(Cl)c1] |
| A208 | 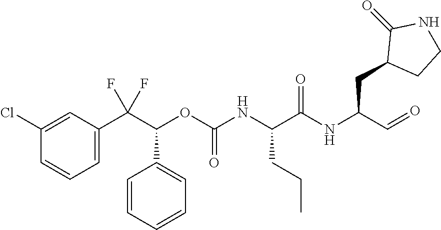 [SMILES: CCC[C@H](NC(=O)O[C@H](c1ccccc1)C(F)(F)c1cccc(Cl)c1)C(=O)N[C@H](C=O)C[C@@H]1CCNC1=O] |
| A209 | 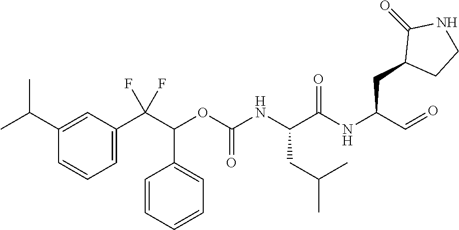 [SMILES: CC(C)C[C@H](NC(=O)OC(c1ccccc1)C(F)(F)c1cccc(C(C)C)c1)C(=O)N[C@H](C=O)C[C@@H]1CCNC1=O] |
| A210 | 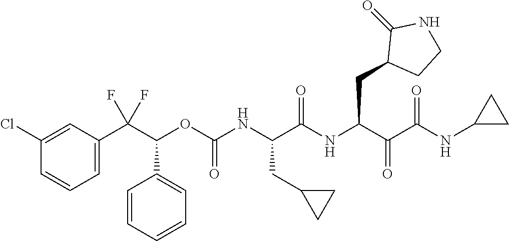 [SMILES: O=C(N[C@@H](CC1CC1)C(=O)N[C@@H](C[C@@H]1CCNC1=O)C(=O)C(=O)NC1CC1)O[C@H](c1ccccc1)C(F)(F)c1cccc(Cl)c1] |
| A211 | 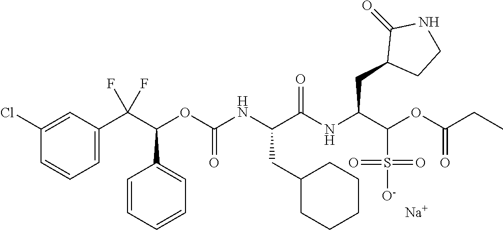 [SMILES: CCC(=O)OC([C@H](C[C@@H]1CCNC1=O)NC(=O)[C@H](CC1CCCCC1)NC(=O)O[C@@H](c1ccccc1)C(F)(F)c1cccc(Cl)c1)S(=O)(=O)[O-] ; [Na+]] |
| A212 | 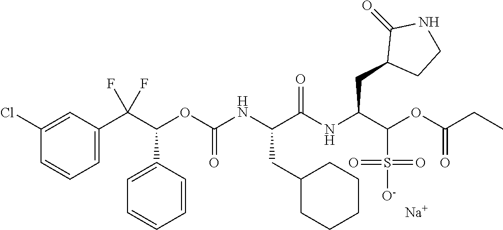 [SMILES: CCC(=O)OC([C@H](C[C@@H]1CCNC1=O)NC(=O)[C@H](CC1CCCCC1)NC(=O)O[C@H](c1ccccc1)C(F)(F)c1cccc(Cl)c1)S(=O)(=O)[O-] ; [Na+]] |
| A213 | 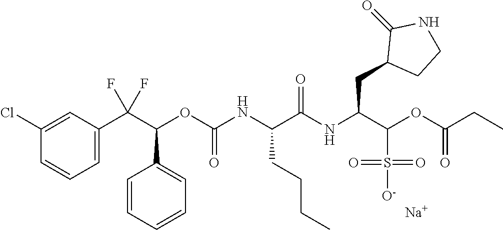 [SMILES: CCCC[C@H](NC(=O)O[C@@H](c1ccccc1)C(F)(F)c1cccc(Cl)c1)C(=O)N[C@@H](C[C@@H]1CCNC1=O)C(OC(=O)CC)S(=O)(=O)[O-] ; [Na+]] |
| A214 | 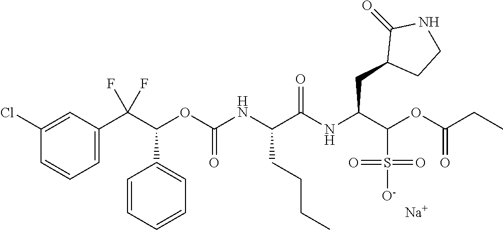 [SMILES: CCCC[C@H](NC(=O)O[C@H](c1ccccc1)C(F)(F)c1cccc(Cl)c1)C(=O)N[C@@H](C[C@@H]1CCNC1=O)C(OC(=O)CC)S(=O)(=O)[O-] ; [Na+]] |
| A215 | 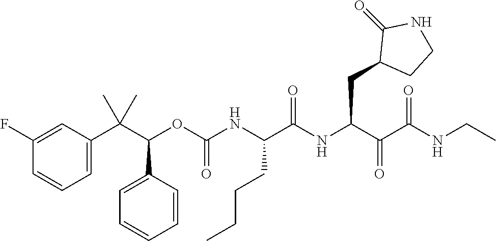 [SMILES: CCCC[C@H](NC(=O)O[C@@H](c1ccccc1)C(C)(C)c1cccc(F)c1)C(=O)N[C@@H](C[C@@H]1CCNC1=O)C(=O)C(=O)NCC] |
| A216 | 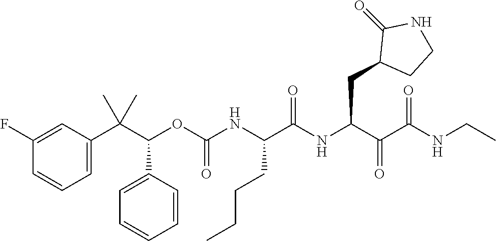 [SMILES: CCCC[C@H](NC(=O)O[C@H](c1ccccc1)C(C)(C)c1cccc(F)c1)C(=O)N[C@@H](C[C@@H]1CCNC1=O)C(=O)C(=O)NCC] |
| A217 | 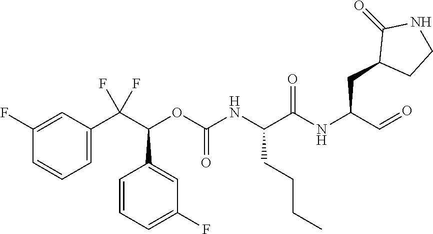 [SMILES: CCCC[C@H](NC(=O)O[C@@H](c1cccc(F)c1)C(F)(F)c1cccc(F)c1)C(=O)N[C@H](C=O)C[C@@H]1CCNC1=O] |
| A218 | 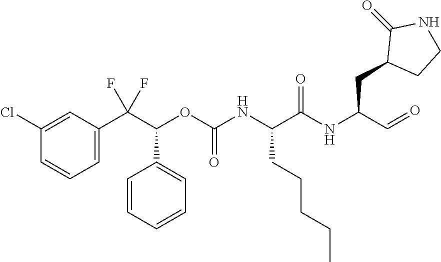 [SMILES: CCCCC[C@H](NC(=O)O[C@H](c1ccccc1)C(F)(F)c1cccc(Cl)c1)C(=O)N[C@H](C=O)C[C@@H]1CCNC1=O] |
| A219 | 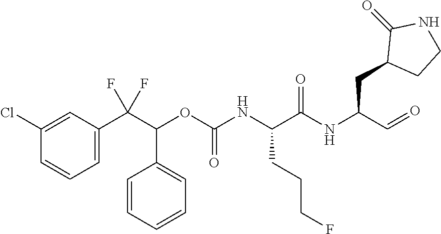 [SMILES: O=C[C@H](C[C@@H]1CCNC1=O)NC(=O)[C@H](CCCF)NC(=O)OC(c1ccccc1)C(F)(F)c1cccc(Cl)c1] |
| A220 | 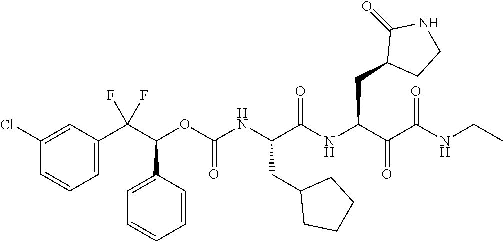 [SMILES: CCNC(=O)C(=O)[C@H](C[C@@H]1CCNC1=O)NC(=O)[C@H](CC1CCCC1)NC(=O)O[C@@H](c1ccccc1)C(F)(F)c1cccc(Cl)c1] |
| A221 | 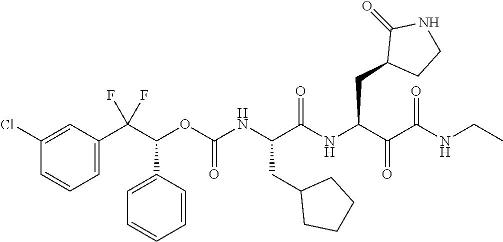 [SMILES: CCNC(=O)C(=O)[C@H](C[C@@H]1CCNC1=O)NC(=O)[C@H](CC1CCCC1)NC(=O)O[C@H](c1ccccc1)C(F)(F)c1cccc(Cl)c1] |
| A222 | 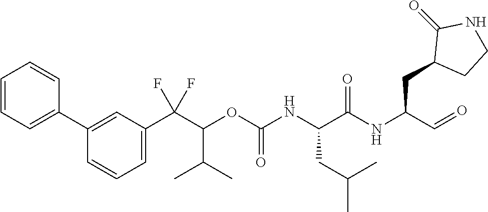 [SMILES: CC(C)C[C@H](NC(=O)OC(C(C)C)C(F)(F)c1cccc(-c2ccccc2)c1)C(=O)N[C@H](C=O)C[C@@H]1CCNC1=O] |
| A223 | 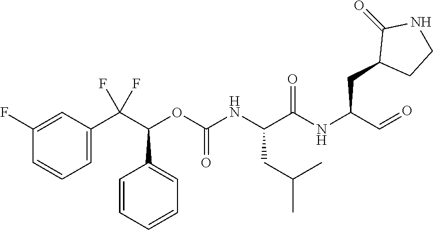 [SMILES: CC(C)C[C@H](NC(=O)O[C@@H](c1ccccc1)C(F)(F)c1cccc(F)c1)C(=O)N[C@H](C=O)C[C@@H]1CCNC1=O] |
| A224 | 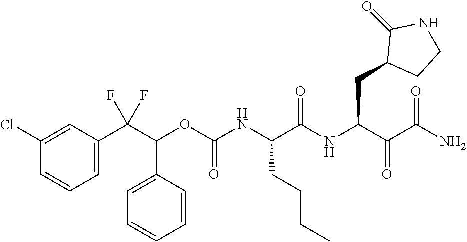 [SMILES: CCCC[C@H](NC(=O)OC(c1ccccc1)C(F)(F)c1cccc(Cl)c1)C(=O)N[C@@H](C[C@@H]1CCNC1=O)C(=O)C(N)=O] |
| A226 | 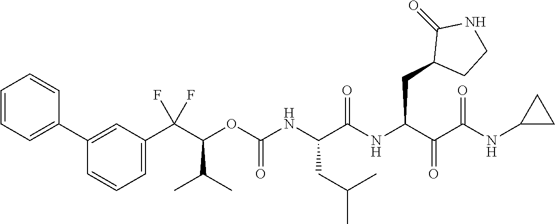 [SMILES: CC(C)C[C@H](NC(=O)O[C@@H](C(C)C)C(F)(F)c1cccc(-c2ccccc2)c1)C(=O)N[C@@H](C[C@@H]1CCNC1=O)C(=O)C(=O)NC1CC1] |
| A227 | 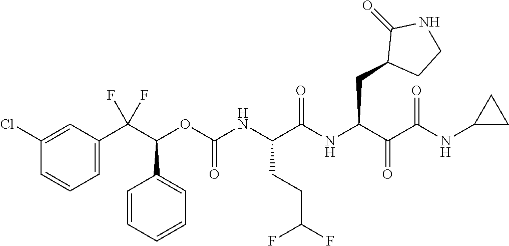 [SMILES: O=C(N[C@@H](CCC(F)F)C(=O)N[C@@H](C[C@@H]1CCNC1=O)C(=O)C(=O)NC1CC1)O[C@@H](c1ccccc1)C(F)(F)c1cccc(Cl)c1] |
| A228 | 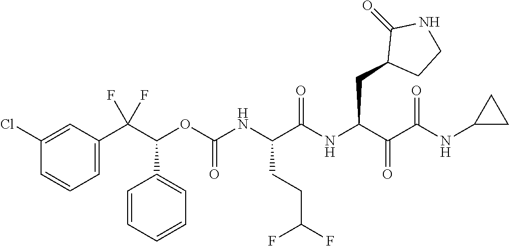 [SMILES: O=C(N[C@@H](CCC(F)F)C(=O)N[C@@H](C[C@@H]1CCNC1=O)C(=O)C(=O)NC1CC1)O[C@H](c1ccccc1)C(F)(F)c1cccc(Cl)c1] |
| A229 | 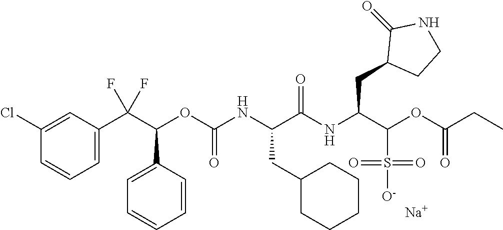 [SMILES: CCC(=O)OC([C@H](C[C@@H]1CCNC1=O)NC(=O)[C@H](CC1CCCCC1)NC(=O)O[C@@H](c1ccccc1)C(F)(F)c1cccc(Cl)c1)S(=O)(=O)[O-] ; [Na+]] |
| A230 | 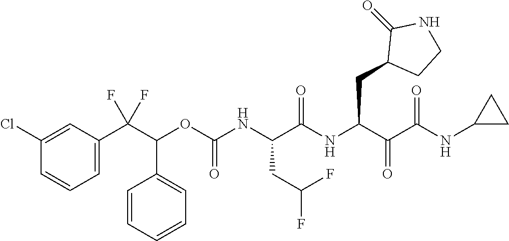 [SMILES: O=C(N[C@@H](CC(F)F)C(=O)N[C@@H](C[C@@H]1CCNC1=O)C(=O)C(=O)NC1CC1)OC(c1ccccc1)C(F)(F)c1cccc(Cl)c1] |
| A231 | 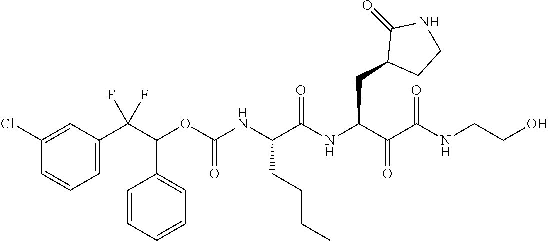 [SMILES: CCCC[C@H](NC(=O)OC(c1ccccc1)C(F)(F)c1cccc(Cl)c1)C(=O)N[C@@H](C[C@@H]1CCNC1=O)C(=O)C(=O)NCCO] |
| A232 | 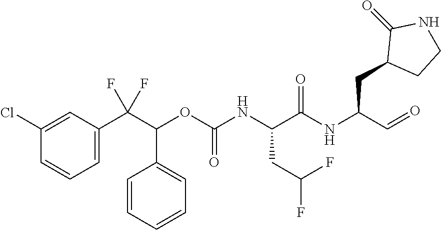 [SMILES: O=C[C@H](C[C@@H]1CCNC1=O)NC(=O)[C@H](CC(F)F)NC(=O)OC(c1ccccc1)C(F)(F)c1cccc(Cl)c1] |
| A233 | 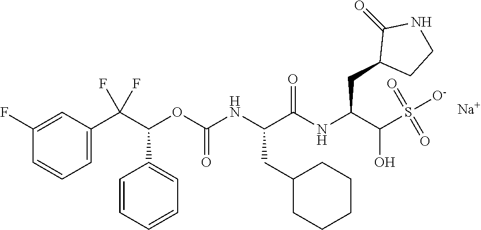 [SMILES: O=C(N[C@@H](CC1CCCCC1)C(=O)N[C@@H](C[C@@H]1CCNC1=O)C(O)S(=O)(=O)[O-])O[C@H](c1ccccc1)C(F)(F)c1cccc(F)c1 ; [Na+]] |
| A234 | 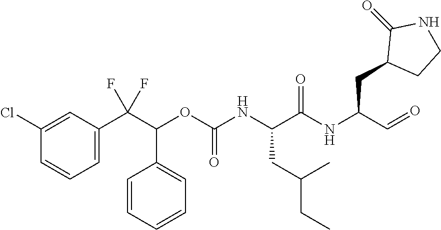 [SMILES: CCC(C)C[C@H](NC(=O)OC(c1ccccc1)C(F)(F)c1cccc(Cl)c1)C(=O)N[C@H](C=O)C[C@@H]1CCNC1=O] |
| A235 | 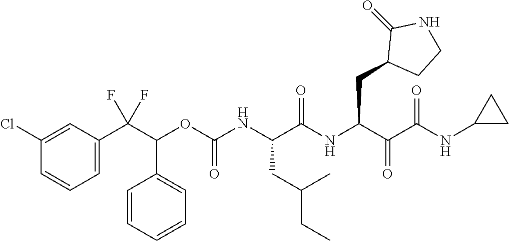 [SMILES: CCC(C)C[C@H](NC(=O)OC(c1ccccc1)C(F)(F)c1cccc(Cl)c1)C(=O)N[C@@H](C[C@@H]1CCNC1=O)C(=O)C(=O)NC1CC1] |
| A236 | 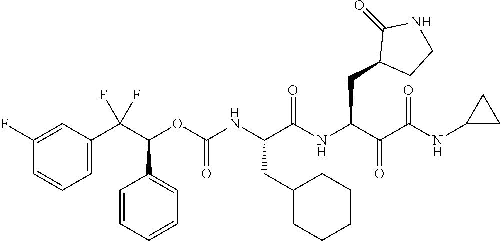 [SMILES: O=C(N[C@@H](CC1CCCCC1)C(=O)N[C@@H](C[C@@H]1CCNC1=O)C(=O)C(=O)NC1CC1)O[C@@H](c1ccccc1)C(F)(F)c1cccc(F)c1] |
| A237 | 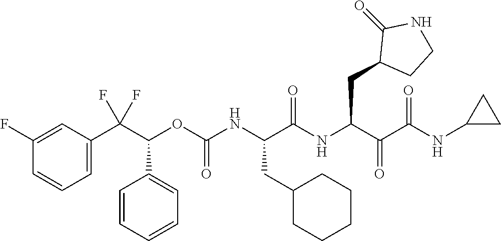 [SMILES: O=C(N[C@@H](CC1CCCCC1)C(=O)N[C@@H](C[C@@H]1CCNC1=O)C(=O)C(=O)NC1CC1)O[C@H](c1ccccc1)C(F)(F)c1cccc(F)c1] |
| A238 | 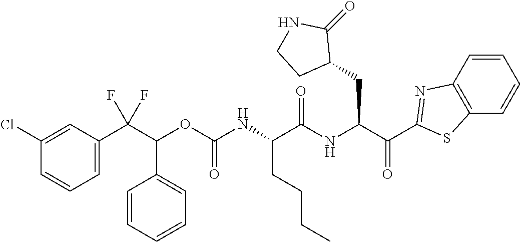 [SMILES: CCCC[C@H](NC(=O)OC(c1ccccc1)C(F)(F)c1cccc(Cl)c1)C(=O)N[C@@H](C[C@@H]1CCNC1=O)C(=O)c1nc2ccccc2s1] |
| A239 | 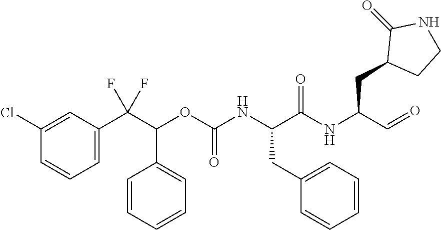 [SMILES: O=C[C@H](C[C@@H]1CCNC1=O)NC(=O)[C@H](Cc1ccccc1)NC(=O)OC(c1ccccc1)C(F)(F)c1cccc(Cl)c1] |
| A240 | 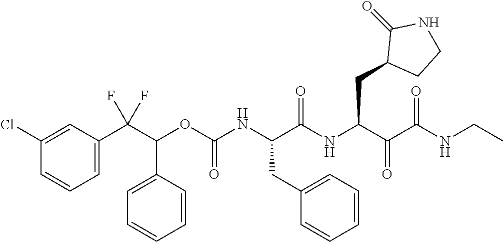 [SMILES: CCNC(=O)C(=O)[C@H](C[C@@H]1CCNC1=O)NC(=O)[C@H](Cc1ccccc1)NC(=O)OC(c1ccccc1)C(F)(F)c1cccc(Cl)c1] |
| A241 | 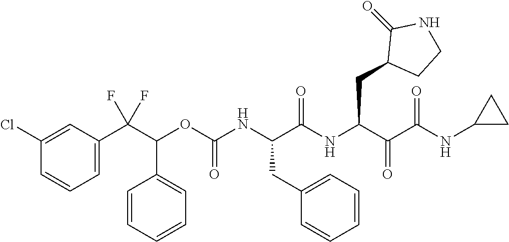 [SMILES: O=C(N[C@@H](Cc1ccccc1)C(=O)N[C@@H](C[C@@H]1CCNC1=O)C(=O)C(=O)NC1CC1)OC(c1ccccc1)C(F)(F)c1cccc(Cl)c1] |
| A242 | 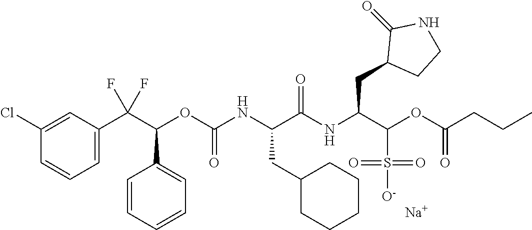 [SMILES: CCCC(=O)OC([C@H](C[C@@H]1CCNC1=O)NC(=O)[C@H](CC1CCCCC1)NC(=O)O[C@@H](c1ccccc1)C(F)(F)c1cccc(Cl)c1)S(=O)(=O)[O-] ; [Na+]] |
| A243 | 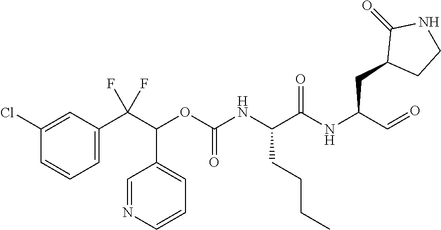 [SMILES: CCCC[C@H](NC(=O)OC(c1cccnc1)C(F)(F)c1cccc(Cl)c1)C(=O)N[C@H](C=O)C[C@@H]1CCNC1=O] |
| A243 | 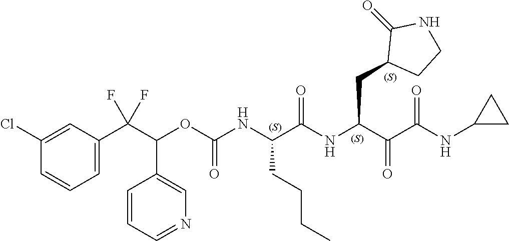 |
| A244 | 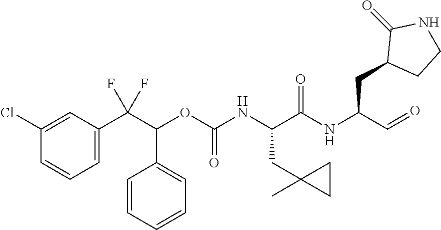 [SMILES: CC1(C[C@H](NC(=O)OC(c2ccccc2)C(F)(F)c2cccc(Cl)c2)C(=O)N[C@H](C=O)C[C@@H]2CCNC2=O)CC1] |
| A245 | 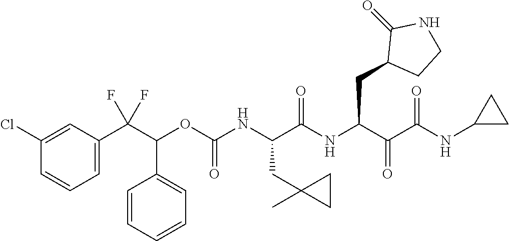 [SMILES: CC1(C[C@H](NC(=O)OC(c2ccccc2)C(F)(F)c2cccc(Cl)c2)C(=O)N[C@@H](C[C@@H]2CCNC2=O)C(=O)C(=O)NC2CC2)CC1] |
| A246 | 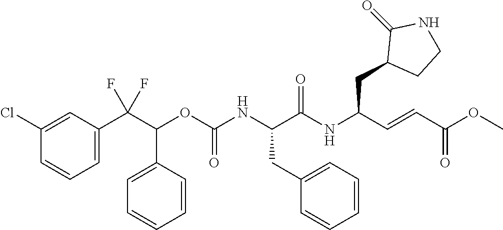 [SMILES: COC(=O)/C=C/[C@H](C[C@@H]1CCNC1=O)NC(=O)[C@H](Cc1ccccc1)NC(=O)OC(c1ccccc1)C(F)(F)c1cccc(Cl)c1] |
| A247 | 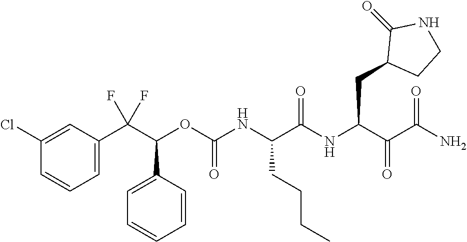 [SMILES: CCCC[C@H](NC(=O)O[C@@H](c1ccccc1)C(F)(F)c1cccc(Cl)c1)C(=O)N[C@@H](C[C@@H]1CCNC1=O)C(=O)C(N)=O] |
| A248 | 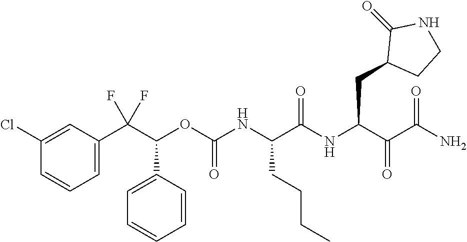 [SMILES: CCCC[C@H](NC(=O)O[C@H](c1ccccc1)C(F)(F)c1cccc(Cl)c1)C(=O)N[C@@H](C[C@@H]1CCNC1=O)C(=O)C(N)=O] |
| A249 | 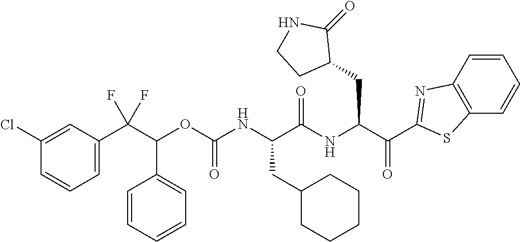 [SMILES: O=C(N[C@@H](CC1CCCCC1)C(=O)N[C@@H](C[C@@H]1CCNC1=O)C(=O)c1nc2ccccc2s1)OC(c1ccccc1)C(F)(F)c1cccc(Cl)c1] |
| A250 | 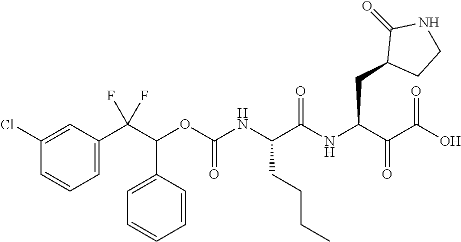 [SMILES: CCCC[C@H](NC(=O)OC(c1ccccc1)C(F)(F)c1cccc(Cl)c1)C(=O)N[C@@H](C[C@@H]1CCNC1=O)C(=O)C(=O)O] |
| A251 | 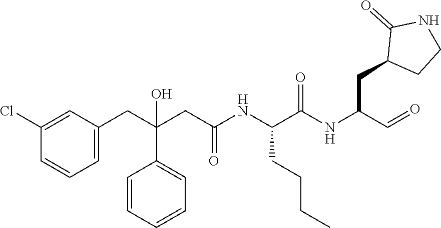 [SMILES: CCCC[C@H](NC(=O)CC(O)(Cc1cccc(Cl)c1)c1ccccc1)C(=O)N[C@H](C=O)C[C@@H]1CCNC1=O] |
| A252 | 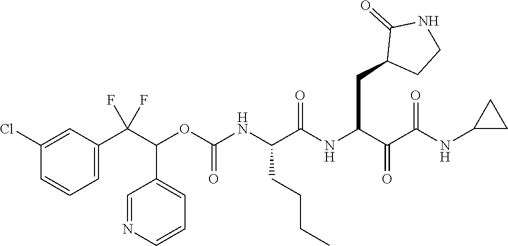 [SMILES: CCCC[C@H](NC(=O)OC(c1cccnc1)C(F)(F)c1cccc(Cl)c1)C(=O)N[C@@H](C[C@@H]1CCNC1=O)C(=O)C(=O)NC1CC1] |
| A253 | 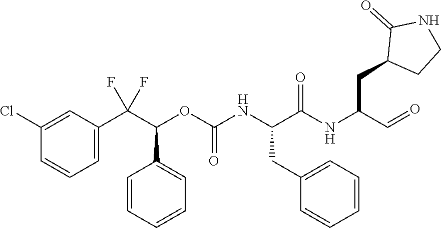 [SMILES: O=C[C@H](C[C@@H]1CCNC1=O)NC(=O)[C@H](Cc1ccccc1)NC(=O)O[C@@H](c1ccccc1)C(F)(F)c1cccc(Cl)c1] |
| A254 | 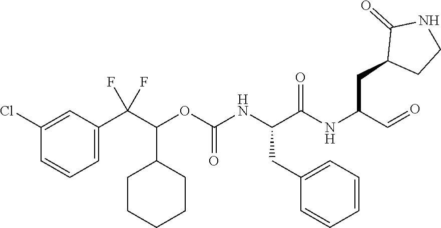 [SMILES: O=C[C@H](C[C@@H]1CCNC1=O)NC(=O)[C@H](Cc1ccccc1)NC(=O)OC(C1CCCCC1)C(F)(F)c1cccc(Cl)c1] |
| A255 | 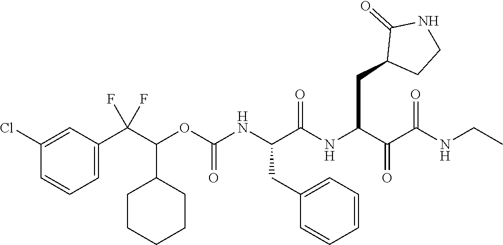 [SMILES: CCNC(=O)C(=O)[C@H](C[C@@H]1CCNC1=O)NC(=O)[C@H](Cc1ccccc1)NC(=O)OC(C1CCCCC1)C(F)(F)c1cccc(Cl)c1] |
| A256 | 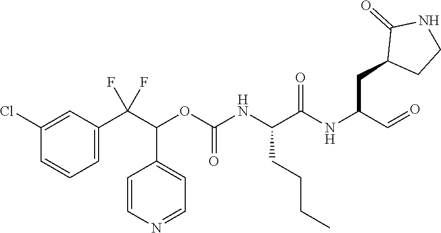 [SMILES: CCCC[C@H](NC(=O)OC(c1ccncc1)C(F)(F)c1cccc(Cl)c1)C(=O)N[C@H](C=O)C[C@@H]1CCNC1=O] |
| A257 | 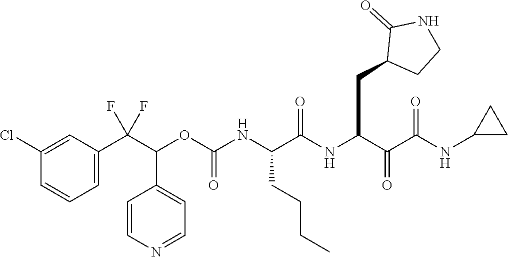 [SMILES: CCCC[C@H](NC(=O)OC(c1ccncc1)C(F)(F)c1cccc(Cl)c1)C(=O)N[C@@H](C[C@@H]1CCNC1=O)C(=O)C(=O)NC1CC1] |
| A258 | 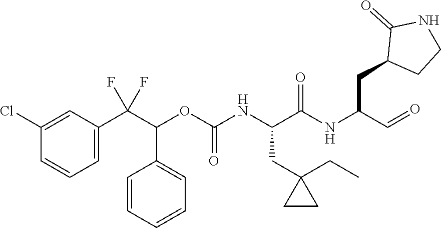 [SMILES: CCC1(C[C@H](NC(=O)OC(c2ccccc2)C(F)(F)c2cccc(Cl)c2)C(=O)N[C@H](C=O)C[C@@H]2CCNC2=O)CC1] |
| A259 | 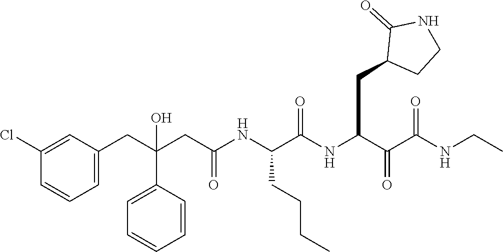 [SMILES: CCCC[C@H](NC(=O)CC(O)(Cc1cccc(Cl)c1)c1ccccc1)C(=O)N[C@@H](C[C@@H]1CCNC1=O)C(=O)C(=O)NCC] |
| A260 | 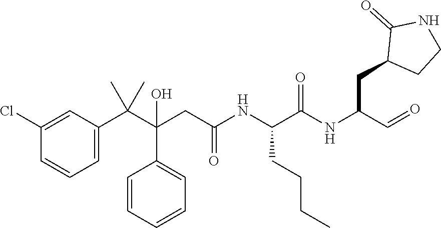 [SMILES: CCCC[C@H](NC(=O)CC(O)(c1ccccc1)C(C)(C)c1cccc(Cl)c1)C(=O)N[C@H](C=O)C[C@@H]1CCNC1=O] |
| A261 | 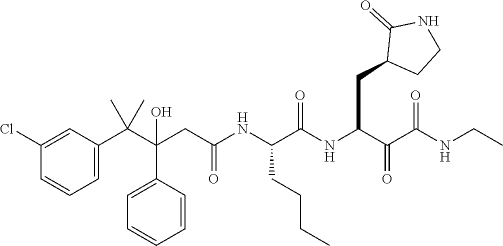 [SMILES: CCCC[C@H](NC(=O)CC(O)(c1ccccc1)C(C)(C)c1cccc(Cl)c1)C(=O)N[C@@H](C[C@@H]1CCNC1=O)C(=O)C(=O)NCC] |
| A262 | 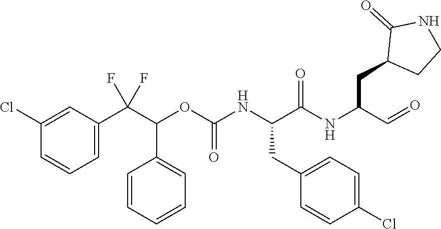 [SMILES: O=C[C@H](C[C@@H]1CCNC1=O)NC(=O)[C@H](Cc1ccc(Cl)cc1)NC(=O)OC(c1ccccc1)C(F)(F)c1cccc(Cl)c1] |
| A263 | 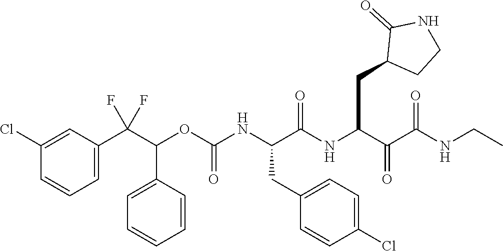 [SMILES: CCNC(=O)C(=O)[C@H](C[C@@H]1CCNC1=O)NC(=O)[C@H](Cc1ccc(Cl)cc1)NC(=O)OC(c1ccccc1)C(F)(F)c1cccc(Cl)c1] |
| A265 | 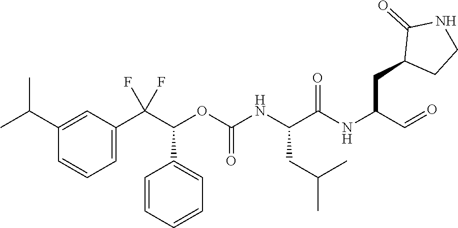 [SMILES: CC(C)C[C@H](NC(=O)O[C@H](c1ccccc1)C(F)(F)c1cccc(C(C)C)c1)C(=O)N[C@H](C=O)C[C@@H]1CCNC1=O] |
| A266 | 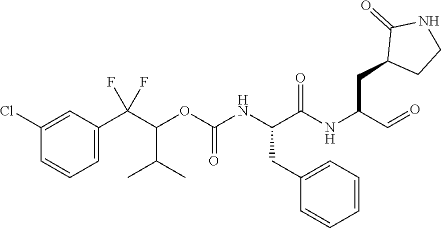 [SMILES: CC(C)C(OC(=O)N[C@@H](Cc1ccccc1)C(=O)N[C@H](C=O)C[C@@H]1CCNC1=O)C(F)(F)c1cccc(Cl)c1] |
| A267 | 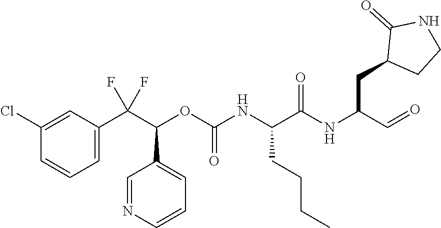 [SMILES: CCCC[C@H](NC(=O)O[C@@H](c1cccnc1)C(F)(F)c1cccc(Cl)c1)C(=O)N[C@H](C=O)C[C@@H]1CCNC1=O] |
| A268 | 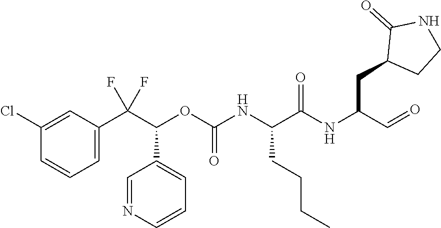 [SMILES: CCCC[C@H](NC(=O)O[C@H](c1cccnc1)C(F)(F)c1cccc(Cl)c1)C(=O)N[C@H](C=O)C[C@@H]1CCNC1=O] |
| A269 | 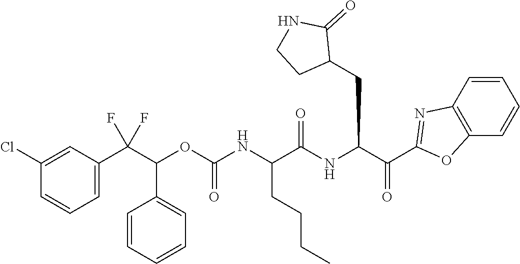 [SMILES: CCCCC(NC(=O)OC(c1ccccc1)C(F)(F)c1cccc(Cl)c1)C(=O)N[C@@H](CC1CCNC1=O)C(=O)c1nc2ccccc2o1] |
| A270 | 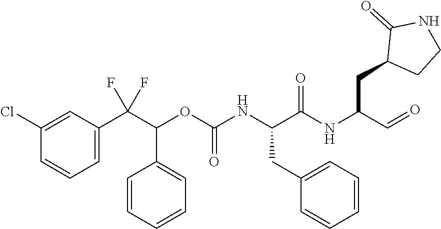 [SMILES: O=C[C@H](C[C@@H]1CCNC1=O)NC(=O)[C@H](Cc1ccccc1)NC(=O)OC(c1ccccc1)C(F)(F)c1cccc(Cl)c1] |
| A271 | 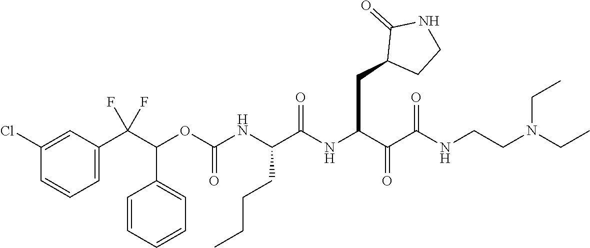 [SMILES: CCCC[C@H](NC(=O)OC(c1ccccc1)C(F)(F)c1cccc(Cl)c1)C(=O)N[C@@H](C[C@@H]1CCNC1=O)C(=O)C(=O)NCCN(CC)CC] |
| A273 | 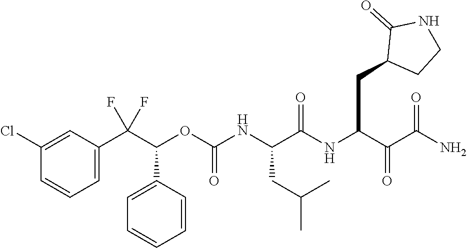 [SMILES: CC(C)C[C@H](NC(=O)O[C@H](c1ccccc1)C(F)(F)c1cccc(Cl)c1)C(=O)N[C@@H](C[C@@H]1CCNC1=O)C(=O)C(N)=O] |
| A274 | 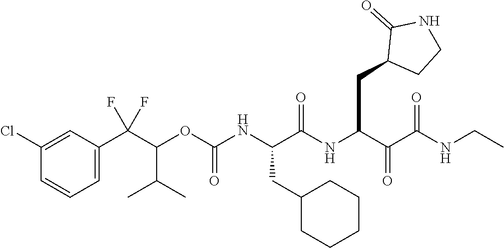 [SMILES: CCNC(=O)C(=O)[C@H](C[C@@H]1CCNC1=O)NC(=O)[C@H](CC1CCCCC1)NC(=O)OC(C(C)C)C(F)(F)c1cccc(Cl)c1] |
| A275 | 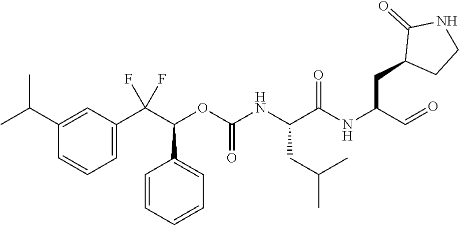 [SMILES: CC(C)C[C@H](NC(=O)O[C@@H](c1ccccc1)C(F)(F)c1cccc(C(C)C)c1)C(=O)N[C@H](C=O)C[C@@H]1CCNC1=O] |
| A278 | 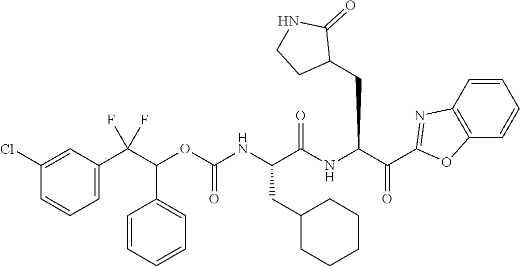 [SMILES: O=C(N[C@@H](CC1CCCCC1)C(=O)N[C@@H](CC1CCNC1=O)C(=O)c1nc2ccccc2o1)OC(c1ccccc1)C(F)(F)c1cccc(Cl)c1] |
| A279 | 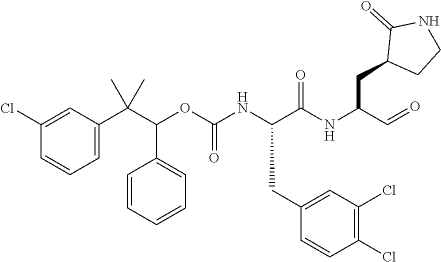 [SMILES: CC(C)(c1cccc(Cl)c1)C(OC(=O)N[C@@H](Cc1ccc(Cl)c(Cl)c1)C(=O)N[C@H](C=O)C[C@@H]1CCNC1=O)c1ccccc1] |
| A280 | 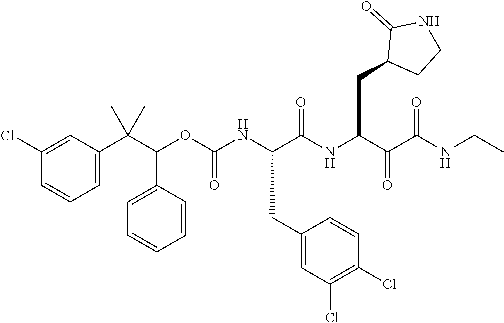 [SMILES: CCNC(=O)C(=O)[C@H](C[C@@H]1CCNC1=O)NC(=O)[C@H](Cc1ccc(Cl)c(Cl)c1)NC(=O)OC(c1ccccc1)C(C)(C)c1cccc(Cl)c1] |
| A281 | 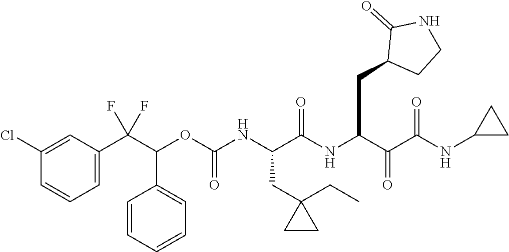 [SMILES: CCC1(C[C@H](NC(=O)OC(c2ccccc2)C(F)(F)c2cccc(Cl)c2)C(=O)N[C@@H](C[C@@H]2CCNC2=O)C(=O)C(=O)NC2CC2)CC1] |
| A282 | 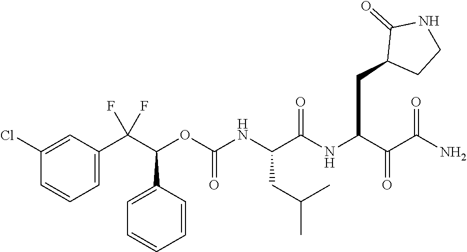 [SMILES: CC(C)C[C@H](NC(=O)O[C@@H](c1ccccc1)C(F)(F)c1cccc(Cl)c1)C(=O)N[C@@H](C[C@@H]1CCNC1=O)C(=O)C(N)=O] |
| A283 | 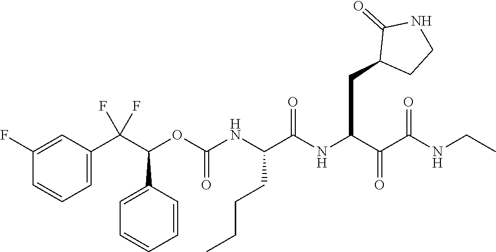 [SMILES: CCCC[C@H](NC(=O)O[C@@H](c1ccccc1)C(F)(F)c1cccc(F)c1)C(=O)N[C@@H](C[C@@H]1CCNC1=O)C(=O)C(=O)NCC] |
| A284 | 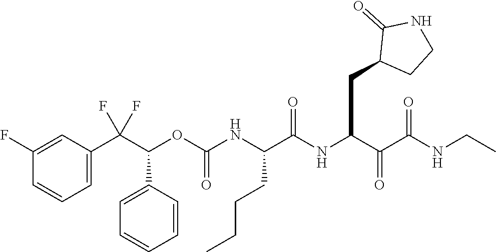 [SMILES: CCCC[C@H](NC(=O)O[C@H](c1ccccc1)C(F)(F)c1cccc(F)c1)C(=O)N[C@@H](C[C@@H]1CCNC1=O)C(=O)C(=O)NCC] |
| A285 | 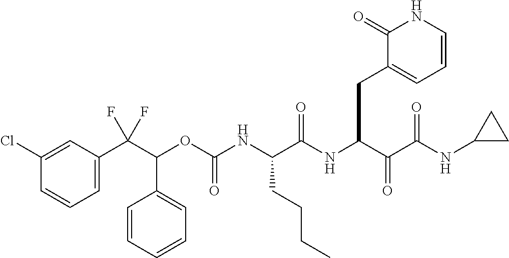 [SMILES: CCCC[C@H](NC(=O)OC(c1ccccc1)C(F)(F)c1cccc(Cl)c1)C(=O)N[C@@H](Cc1ccc[nH]c1=O)C(=O)C(=O)NC1CC1] |
| A286 | 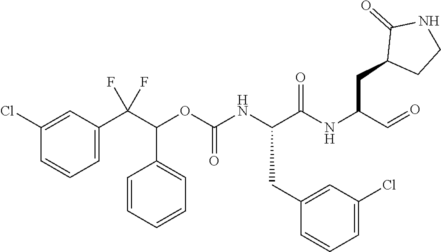 [SMILES: O=C[C@H](C[C@@H]1CCNC1=O)NC(=O)[C@H](Cc1cccc(Cl)c1)NC(=O)OC(c1ccccc1)C(F)(F)c1cccc(Cl)c1] |
| A287 | 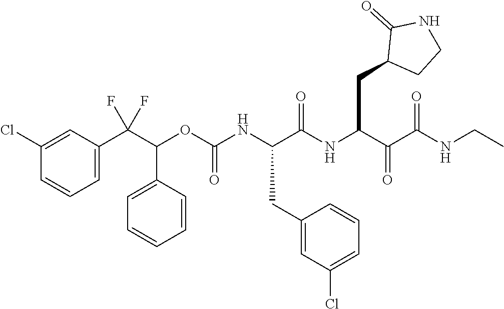 [SMILES: CCNC(=O)C(=O)[C@H](C[C@@H]1CCNC1=O)NC(=O)[C@H](Cc1cccc(Cl)c1)NC(=O)OC(c1ccccc1)C(F)(F)c1cccc(Cl)c1] |
| A288 | 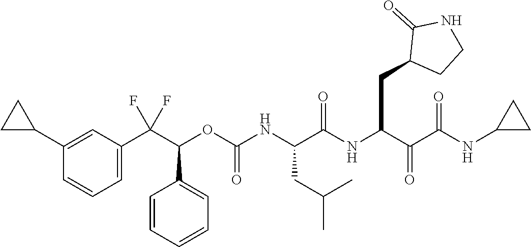 [SMILES: CC(C)C[C@H](NC(=O)O[C@@H](c1ccccc1)C(F)(F)c1cccc(C2CC2)c1)C(=O)N[C@@H](C[C@@H]1CCNC1=O)C(=O)C(=O)NC1CC1] |
| A289 | 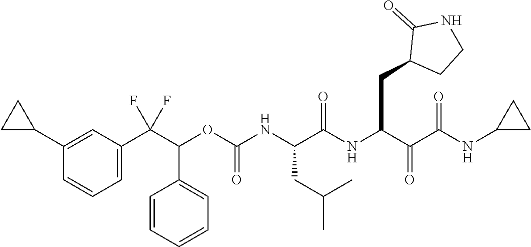 [SMILES: CC(C)C[C@H](NC(=O)OC(c1ccccc1)C(F)(F)c1cccc(C2CC2)c1)C(=O)N[C@@H](C[C@@H]1CCNC1=O)C(=O)C(=O)NC1CC1] |
| A290 | 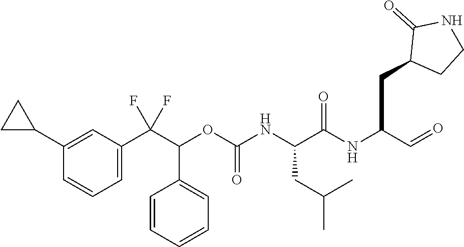 [SMILES: CC(C)C[C@H](NC(=O)OC(c1ccccc1)C(F)(F)c1cccc(C2CC2)c1)C(=O)N[C@H](C=O)C[C@@H]1CCNC1=O] |
| A291 | 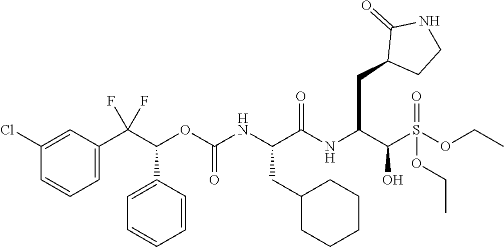 |
| A292 | 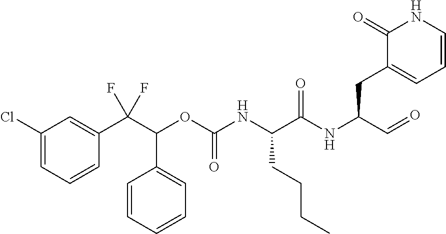 [SMILES: CCCC[C@H](NC(=O)OC(c1ccccc1)C(F)(F)c1cccc(Cl)c1)C(=O)N[C@H](C=O)Cc1ccc[nH]c1=O] |
| A293 | 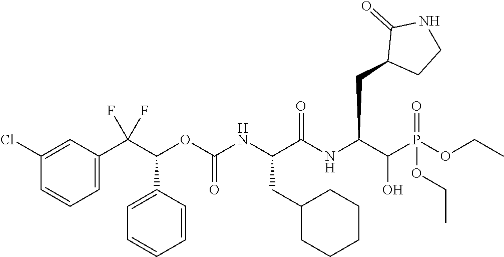 [SMILES: CCOP(=O)(OCC)C(O)[C@H](C[C@@H]1CCNC1=O)NC(=O)[C@H](CC1CCCCC1)NC(=O)O[C@H](c1ccccc1)C(F)(F)c1cccc(Cl)c1] |
| A294 | 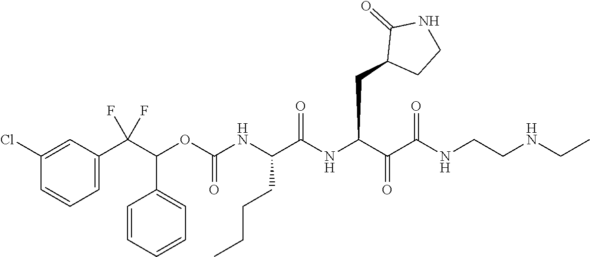 [SMILES: CCCC[C@H](NC(=O)OC(c1ccccc1)C(F)(F)c1cccc(Cl)c1)C(=O)N[C@@H](C[C@@H]1CCNC1=O)C(=O)C(=O)NCCNCC] |
| A295 | 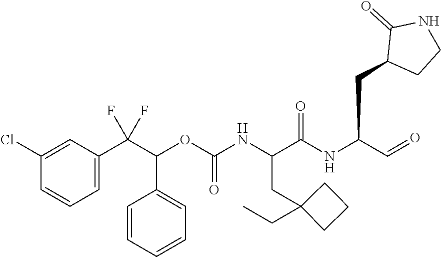 [SMILES: CCC1(CC(NC(=O)OC(c2ccccc2)C(F)(F)c2cccc(Cl)c2)C(=O)N[C@H](C=O)C[C@@H]2CCNC2=O)CCC1] |
| A296 | 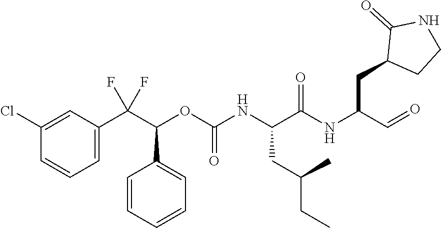 [SMILES: CC[C@H](C)C[C@H](NC(=O)O[C@@H](c1ccccc1)C(F)(F)c1cccc(Cl)c1)C(=O)N[C@H](C=O)C[C@@H]1CCNC1=O] |
| A297 | 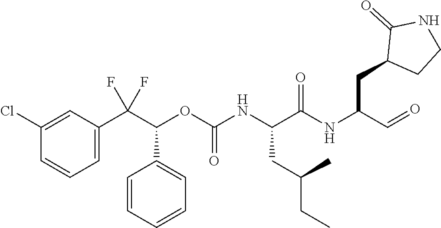 [SMILES: CC[C@H](C)C[C@H](NC(=O)O[C@H](c1ccccc1)C(F)(F)c1cccc(Cl)c1)C(=O)N[C@H](C=O)C[C@@H]1CCNC1=O] |
| A298 | 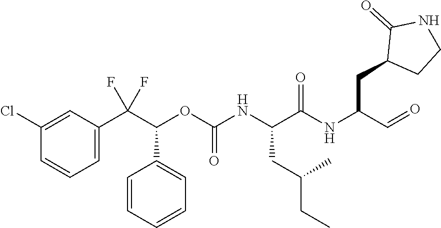 [SMILES: CC[C@@H](C)C[C@H](NC(=O)O[C@H](c1ccccc1)C(F)(F)c1cccc(Cl)c1)C(=O)N[C@H](C=O)C[C@@H]1CCNC1=O] |
| A299 | 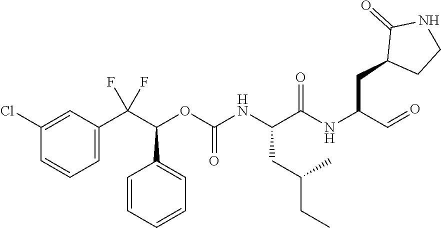 [SMILES: CC[C@@H](C)C[C@H](NC(=O)O[C@@H](c1ccccc1)C(F)(F)c1cccc(Cl)c1)C(=O)N[C@H](C=O)C[C@@H]1CCNC1=O] |
| A300 | 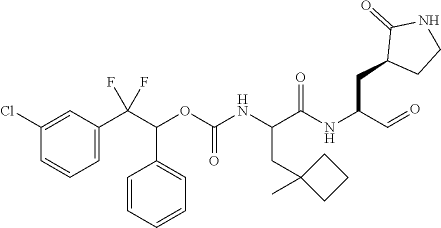 [SMILES: CC1(CC(NC(=O)OC(c2ccccc2)C(F)(F)c2cccc(Cl)c2)C(=O)N[C@H](C=O)C[C@@H]2CCNC2=O)CCC1] |
| A301 | 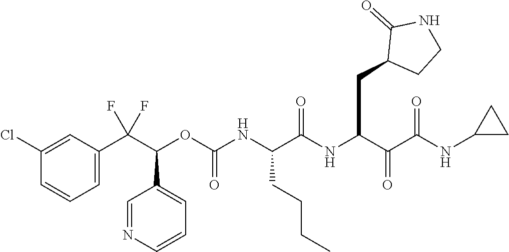 [SMILES: CCCC[C@H](NC(=O)O[C@@H](c1cccnc1)C(F)(F)c1cccc(Cl)c1)C(=O)N[C@@H](C[C@@H]1CCNC1=O)C(=O)C(=O)NC1CC1] |
| A302 | 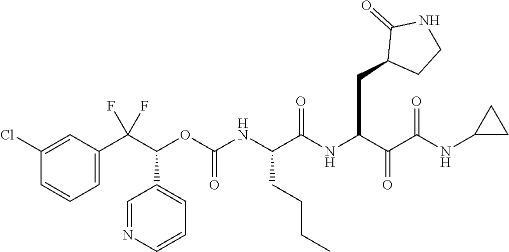 [SMILES: CCCC[C@H](NC(=O)O[C@H](c1cccnc1)C(F)(F)c1cccc(Cl)c1)C(=O)N[C@@H](C[C@@H]1CCNC1=O)C(=O)C(=O)NC1CC1] |
| A303 | 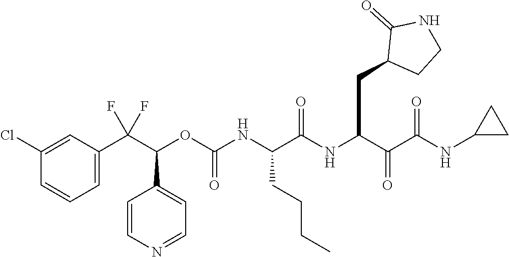 [SMILES: CCCC[C@H](NC(=O)O[C@@H](c1ccncc1)C(F)(F)c1cccc(Cl)c1)C(=O)N[C@@H](C[C@@H]1CCNC1=O)C(=O)C(=O)NC1CC1] |
| A304 | 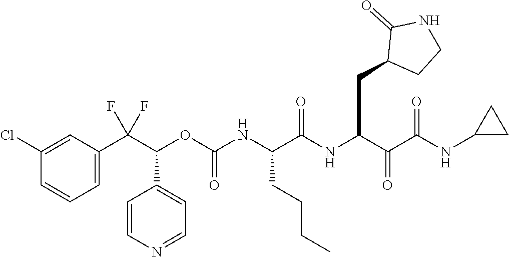 [SMILES: CCCC[C@H](NC(=O)O[C@H](c1ccncc1)C(F)(F)c1cccc(Cl)c1)C(=O)N[C@@H](C[C@@H]1CCNC1=O)C(=O)C(=O)NC1CC1] |
| A305 | 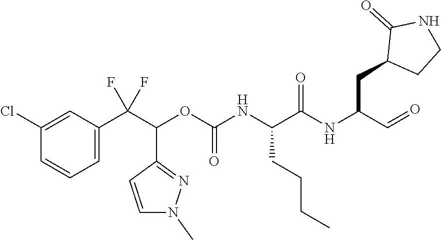 [SMILES: CCCC[C@H](NC(=O)OC(c1ccn(C)n1)C(F)(F)c1cccc(Cl)c1)C(=O)N[C@H](C=O)C[C@@H]1CCNC1=O] |
| A306 | 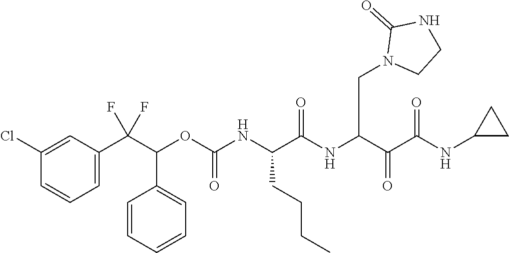 [SMILES: CCCC[C@H](NC(=O)OC(c1ccccc1)C(F)(F)c1cccc(Cl)c1)C(=O)NC(CN1CCNC1=O)C(=O)C(=O)NC1CC1] |
| A307 | 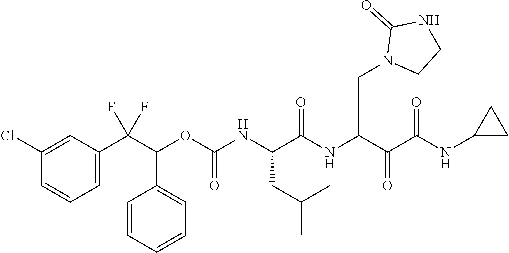 [SMILES: CC(C)C[C@H](NC(=O)OC(c1ccccc1)C(F)(F)c1cccc(Cl)c1)C(=O)NC(CN1CCNC1=O)C(=O)C(=O)NC1CC1] |
| A308 | 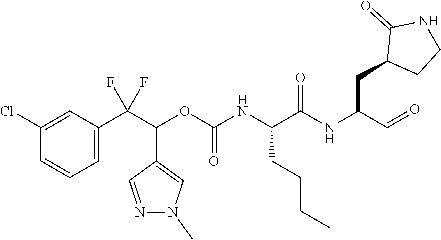 [SMILES: CCCC[C@H](NC(=O)OC(c1cnn(C)c1)C(F)(F)c1cccc(Cl)c1)C(=O)N[C@H](C=O)C[C@@H]1CCNC1=O] |
| A309 | 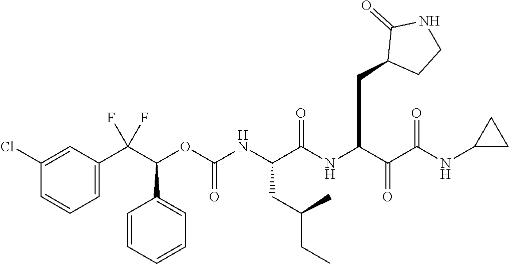 [SMILES: CC[C@H](C)C[C@H](NC(=O)O[C@@H](c1ccccc1)C(F)(F)c1cccc(Cl)c1)C(=O)N[C@@H](C[C@@H]1CCNC1=O)C(=O)C(=O)NC1CC1] |
| A310 | 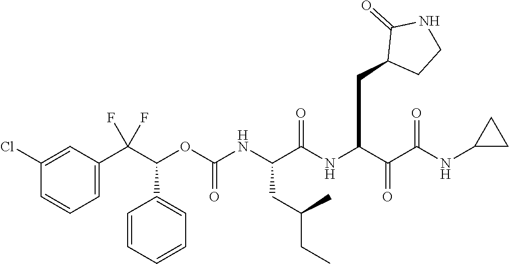 [SMILES: CC[C@H](C)C[C@H](NC(=O)O[C@H](c1ccccc1)C(F)(F)c1cccc(Cl)c1)C(=O)N[C@@H](C[C@@H]1CCNC1=O)C(=O)C(=O)NC1CC1] |
| A311 | 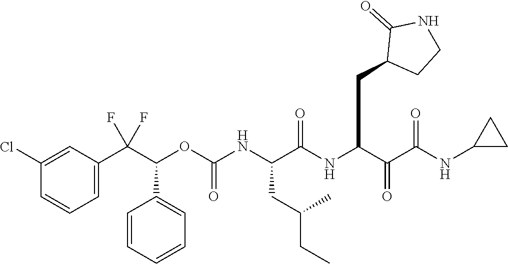 [SMILES: CC[C@@H](C)C[C@H](NC(=O)O[C@H](c1ccccc1)C(F)(F)c1cccc(Cl)c1)C(=O)N[C@@H](C[C@@H]1CCNC1=O)C(=O)C(=O)NC1CC1] |
| A312 | 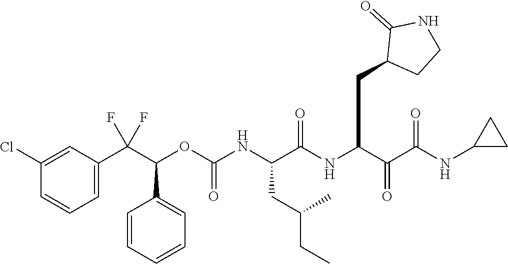 [SMILES: CC[C@@H](C)C[C@H](NC(=O)O[C@@H](c1ccccc1)C(F)(F)c1cccc(Cl)c1)C(=O)N[C@@H](C[C@@H]1CCNC1=O)C(=O)C(=O)NC1CC1] |
| A313 | 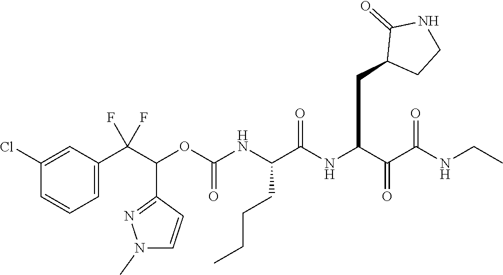 [SMILES: CCCC[C@H](NC(=O)OC(c1ccn(C)n1)C(F)(F)c1cccc(Cl)c1)C(=O)N[C@@H](C[C@@H]1CCNC1=O)C(=O)C(=O)NCC] |
| A314 | 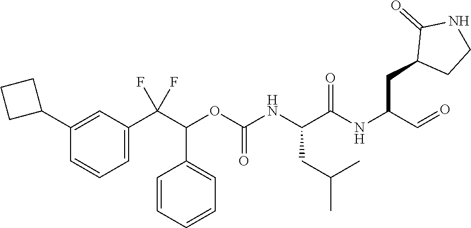 [SMILES: CC(C)C[C@H](NC(=O)OC(c1ccccc1)C(F)(F)c1cccc(C2CCC2)c1)C(=O)N[C@H](C=O)C[C@@H]1CCNC1=O] |
| A315 | 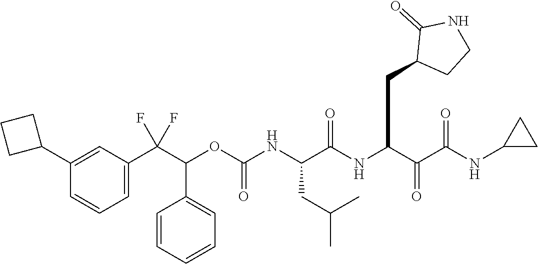 [SMILES: CC(C)C[C@H](NC(=O)OC(c1ccccc1)C(F)(F)c1cccc(C2CCC2)c1)C(=O)N[C@@H](C[C@@H]1CCNC1=O)C(=O)C(=O)NC1CC1] |
| A316 | 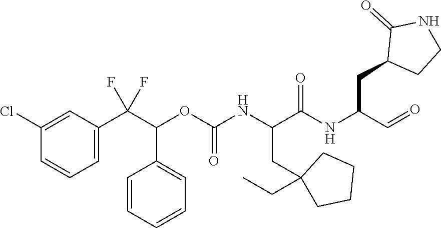 [SMILES: CCC1(CC(NC(=O)OC(c2ccccc2)C(F)(F)c2cccc(Cl)c2)C(=O)N[C@H](C=O)C[C@@H]2CCNC2=O)CCCC1] |
| A317 | 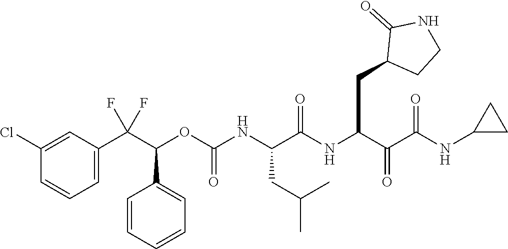 [SMILES: CC(C)C[C@H](NC(=O)O[C@@H](c1ccccc1)C(F)(F)c1cccc(Cl)c1)C(=O)N[C@@H](C[C@@H]1CCNC1=O)C(=O)C(=O)NC1CC1] |
| A318 | 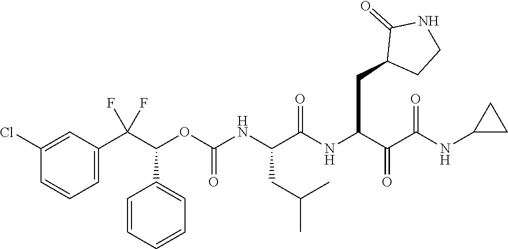 [SMILES: CC(C)C[C@H](NC(=O)O[C@H](c1ccccc1)C(F)(F)c1cccc(Cl)c1)C(=O)N[C@@H](C[C@@H]1CCNC1=O)C(=O)C(=O)NC1CC1] |
| A319 | 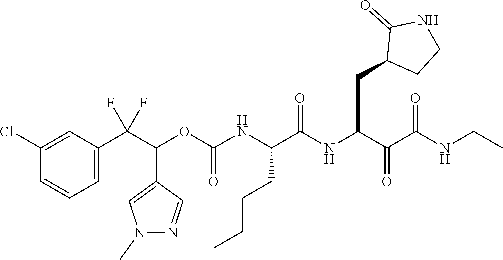 [SMILES: CCCC[C@H](NC(=O)OC(c1cnn(C)c1)C(F)(F)c1cccc(Cl)c1)C(=O)N[C@@H](C[C@@H]1CCNC1=O)C(=O)C(=O)NCC] |
| A320 | 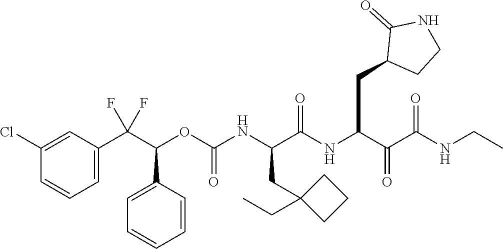 [SMILES: CCNC(=O)C(=O)[C@H](C[C@@H]1CCNC1=O)NC(=O)[C@@H](CC1(CC)CCC1)NC(=O)O[C@@H](c1ccccc1)C(F)(F)c1cccc(Cl)c1] |
| A321 | 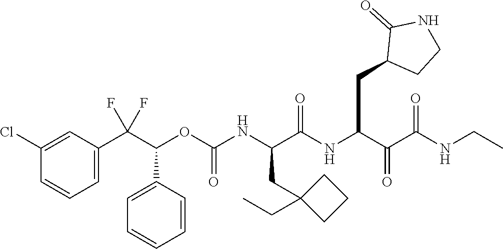 [SMILES: CCNC(=O)C(=O)[C@H](C[C@@H]1CCNC1=O)NC(=O)[C@@H](CC1(CC)CCC1)NC(=O)O[C@H](c1ccccc1)C(F)(F)c1cccc(Cl)c1] |
| A322 | 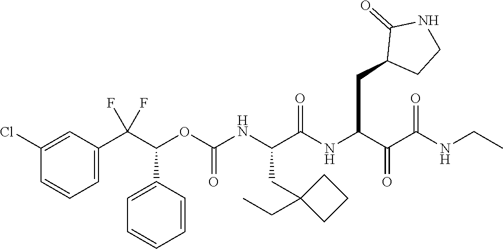 [SMILES: CCNC(=O)C(=O)[C@H](C[C@@H]1CCNC1=O)NC(=O)[C@H](CC1(CC)CCC1)NC(=O)O[C@H](c1ccccc1)C(F)(F)c1cccc(Cl)c1] |
| A323 | 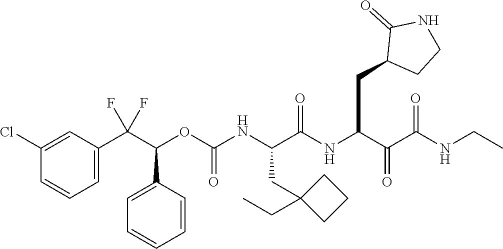 [SMILES: CCNC(=O)C(=O)[C@H](C[C@@H]1CCNC1=O)NC(=O)[C@H](CC1(CC)CCC1)NC(=O)O[C@@H](c1ccccc1)C(F)(F)c1cccc(Cl)c1] |
| A324 | 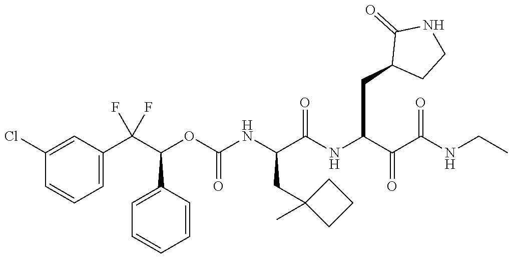 [SMILES: CCNC(=O)C(=O)[C@H](C[C@@H]1CCNC1=O)NC(=O)[C@@H](CC1(C)CCC1)NC(=O)O[C@@H](c1ccccc1)C(F)(F)c1cccc(Cl)c1] |
| A325 | 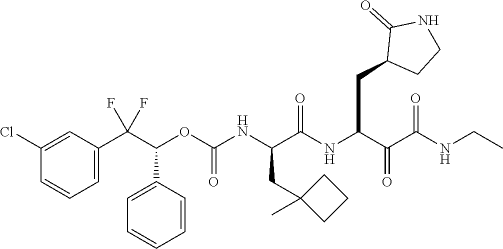 [SMILES: CCNC(=O)C(=O)[C@H](C[C@@H]1CCNC1=O)NC(=O)[C@@H](CC1(C)CCC1)NC(=O)O[C@H](c1ccccc1)C(F)(F)c1cccc(Cl)c1] |
| A326 | 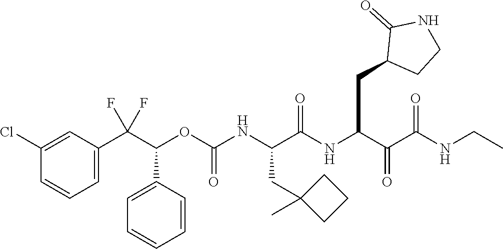 [SMILES: CCNC(=O)C(=O)[C@H](C[C@@H]1CCNC1=O)NC(=O)[C@H](CC1(C)CCC1)NC(=O)O[C@H](c1ccccc1)C(F)(F)c1cccc(Cl)c1] |
| A327 | 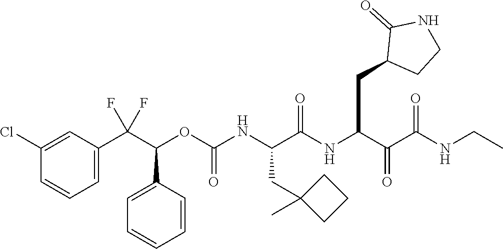 [SMILES: CCNC(=O)C(=O)[C@H](C[C@@H]1CCNC1=O)NC(=O)[C@H](CC1(C)CCC1)NC(=O)O[C@@H](c1ccccc1)C(F)(F)c1cccc(Cl)c1] |
| A328 | 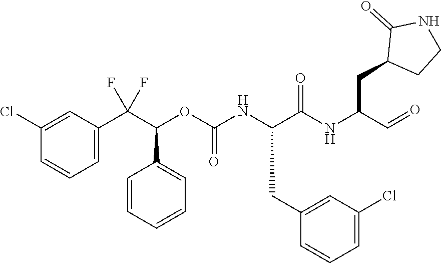 [SMILES: O=C[C@H](C[C@@H]1CCNC1=O)NC(=O)[C@H](Cc1cccc(Cl)c1)NC(=O)O[C@@H](c1ccccc1)C(F)(F)c1cccc(Cl)c1] |
| A329 | 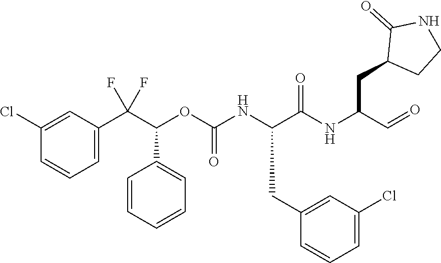 [SMILES: O=C[C@H](C[C@@H]1CCNC1=O)NC(=O)[C@H](Cc1cccc(Cl)c1)NC(=O)O[C@H](c1ccccc1)C(F)(F)c1cccc(Cl)c1] |

TABLE B

| Compound | |
|---|---|
| no. | Structure |
| A8 | 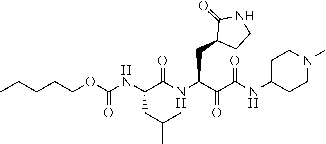 [SMILES: CCCCCOC(=O)N[C@@H](CC(C)C)C(=O)N[C@@H](C[C@@H]1CCNC1=O)C(=O)C(=O)NC1CCN(C)CC1] |
| A12 | 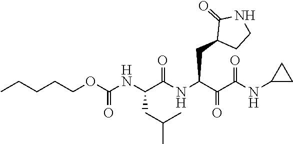 [SMILES: CCCCCOC(=O)N[C@@H](CC(C)C)C(=O)N[C@@H](C[C@@H]1CCNC1=O)C(=O)C(=O)NC1CC1] |
| A13 | 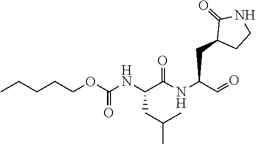 [SMILES: CCCCCOC(=O)N[C@@H](CC(C)C)C(=O)N[C@H](C=O)C[C@@H]1CCNC1=O] |
| A24 | 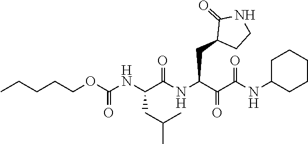 [SMILES: CCCCCOC(=O)N[C@@H](CC(C)C)C(=O)N[C@@H](C[C@@H]1CCNC1=O)C(=O)C(=O)NC1CCCCC1] |
| A150 | 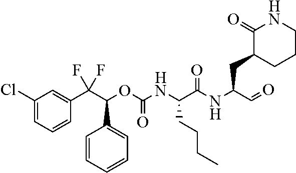 [SMILES: CCCC[C@H](NC(=O)O[C@@H](c1ccccc1)C(F)(F)c1cccc(Cl)c1)C(=O)N[C@H](C=O)C[C@@H]1CCCNC1=O] |
| A158 | 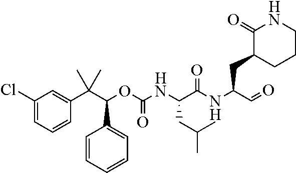 [SMILES: CC(C)C[C@H](NC(=O)O[C@@H](c1ccccc1)C(C)(C)c1cccc(Cl)c1)C(=O)N[C@H](C=O)C[C@@H]1CCCNC1=O] |
| A264 | 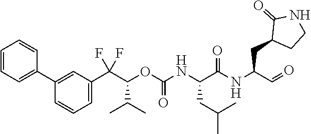 [SMILES: CC(C)C[C@H](NC(=O)O[C@H](C(C)C)C(F)(F)c1cccc(-c2ccccc2)c1)C(=O)N[C@H](C=O)C[C@@H]1CCNC1=O] |
| A272 | 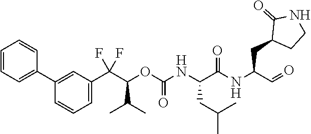 [SMILES: CC(C)C[C@H](NC(=O)O[C@@H](C(C)C)C(F)(F)c1cccc(-c2ccccc2)c1)C(=O)N[C@H](C=O)C[C@@H]1CCNC1=O] |
| A276 | 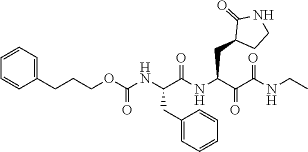 [SMILES: CCNC(=O)C(=O)[C@H](C[C@@H]1CCNC1=O)NC(=O)[C@H](Cc1ccccc1)NC(=O)OCCCc1ccccc1] |
| A277 | 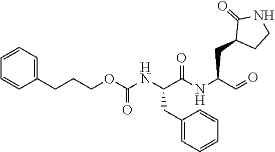 [SMILES: O=C[C@H](C[C@@H]1CCNC1=O)NC(=O)[C@H](Cc1ccccc1)NC(=O)OCCCc1ccccc1] |

In some cases, the compound is selected from Compounds A20 and A25-A29, or a pharmaceutically acceptable salt thereof. In some cases, the compound is selected from A20, A20-1, A20-2, A25-1, A48, A72, A119, A126, A164, A165, A167, A187, A196, A206, A209, A210, A228, A235, A239, A258, A265, A268, A270, A273, A289, A290, A295, A296, A297, A298, A299, A300, A302, A308, A309, A310, A316, A321, and A325, or a pharmaceutically acceptable salt thereof.

The compounds disclosed herein can be useful as inhibitors of norovirus or coronavirus replication in biological samples or in a patient. These compounds can also be useful in reducing the amount of noroviruses or coronaviruses (viral titer) in a biological sample or in a patient. They can also be useful for therapeutic and prophylactic treatment of infections caused by the noroviruses or coronaviruses in a biological sample or in a patient.

### Pharmaceutically Acceptable Salts

The compounds described herein can exist in free form, or, where appropriate, as salts. Those salts that are pharmaceutically acceptable are of particular interest since they are useful in administering the compounds described below for medical purposes. Salts that are not pharmaceutically acceptable are useful in manufacturing processes, for isolation and purification purposes, and in some instances, for use in separating stereoisomeric forms of the compounds of the disclosure or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use.

Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Compositions

The compounds described herein can be formulated into pharmaceutical compositions that further comprise a pharmaceutically acceptable carrier, diluent, adjuvant or vehicle. In embodiments, the present disclosure relates to a pharmaceutical composition comprising a compound described above or salt thereof, and a pharmaceutically acceptable carrier, diluent, adjuvant or vehicle. In embodiments, the pharmaceutical composition comprises a safe and effective amount of a compound as disclosed herein or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

An “effective amount” includes a “therapeutically effective amount” and a “prophylactically effective amount”. The term “therapeutically effective amount” refers to an amount effective in treating and/or ameliorating a norovirus or coronavirus virus infection in a patient. The term “prophylactically effective amount” refers to an amount effective in preventing and/or substantially lessening the chances or the size of norovirus or coronavirus virus infection outbreak.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this disclosure. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Formulations for Pulmonary Delivery

In some embodiments, the pharmaceutical compositions disclosed herein are adapted to be administered to the lower respiratory tract (e.g., the lungs) directly through the airways by inhalation. Compositions for administration by inhalation may take the form of inhalable powder compositions or liquid or powder sprays, and can be administered in standard form using powder inhaler devices or aerosol dispensing devices. Such devices are well known. For administration by inhalation, the powdered formulations typically comprise the active compound together with an inert solid powdered diluent such as lactose or starch. Inhalable dry powder compositions may be presented in capsules and cartridges of gelatin or a like material, or blisters of laminated aluminum foil for use in an inhaler or insufflators. Each capsule or cartridge may generally contain e.g., from about 10 mg to about 100 g of each active compound. Alternatively, the composition may be presented without excipients.

The inhalable compositions may be packaged for unit dose or multi-dose delivery. For example, the compositions can be packaged for multi-dose delivery in a manner analogous to that described in GB 2242134, U.S. Pat. Nos. 6,632,666, 5,860,419, 5,873,360, and 5,590,645 (all illustrating the “Diskus” device), or GB2i78965, GB2129691, GB2169265, U.S. Pat. Nos. 4,778,054, 4,811,731 and 5,035,237 (which illustrate the “Diskhaler” device), or EP 69715 (“Turbuhaler” device), or GB 2064336 and U.S. Pat. No. 4,353,656 (“Rotahaler” device).

Spray compositions for topical delivery to the lung by inhalation may be formulated as aqueous solutions or suspensions or as aerosols delivered from pressurized packs, such as a metered dose inhaler (MDI), with the use of a suitable liquefied propellant, including hydrofluoroalkanes such as dichlorodifluoromethane, trichlorofluoromethane, dichlorotetrafluoroethane, and especially 1,1,1,2-tetrafluoroethane, 1,1,1,2,3,3,3-heptafluoro-n-propane and mixtures thereof. Aerosol compositions suitable for inhalation can be presented either as suspensions or as solutions.

Medicaments for administration by inhalation typically have a controlled particle size. The optimum particle size for inhalation into the bronchial system is usually about 1 to about 10 μm, and in some embodiments, from about 2 to about 5 μm. Particles having a size above about 20 μm are generally too large when inhaled to reach the small airways. To achieve these particle sizes the particles of the active ingredient may be subjected to a size reducing process such as micronization. The desired size fraction may be separated out by air classification or sieving. Preferably, the particles will be crystalline.

Intranasal sprays may be formulated with aqueous or non-aqueous vehicles with the addition of agents such as thickening agents, buffer salts or acid or alkali to adjust the pH, isotonic adjusting agents or antioxidants.

Solutions for inhalation by nebulization may be formulated with an aqueous vehicle with the addition of agents such as acid or alkali, buffer salts, isotonic adjusting agents or antimicrobial agents. They may be sterilized by filtration or heating in an autoclave, or presented as a non-sterile product. Nebulizers supply the aerosol as a mist created from an aqueous formulation.

In some embodiments, the pharmaceutical compositions disclosed herein can be formulated with supplementary active ingredients.

In some embodiments, the pharmaceutical composition disclosed herein is administered from a dry powder inhaler. In other embodiments, the pharmaceutical composition disclosed herein is administered by an aerosol dispensing device, optionally in conjunction with an inhalation chamber such as the “Volumatic” ® inhalation chamber.

The carrier can be a solvent or dispersion medium containing, for example, water, ethanol, polyol (for example, glycerol, propylene glycol and liquid polyethylene glycol, and the like), suitable mixtures thereof, and/or vegetable oils. The proper fluidity can be maintained, for example, by the use of a coating such as, for example, lecithin, by the maintenance of the required particle size in the case of dispersion and by the use of surfactants. Preventing the action of microorganisms in the compositions disclosed herein is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, a pharmaceutical composition can be within a matrix which controls the release of the composition. In some embodiments, the matrix can comprise lipid, polyvinyl alcohol, polyvinyl acetate, polycaprolactone, poly(glycolic)acid, poly(lactic)acid, polycaprolactone, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and combinations thereof and other polymers such as those disclosed, for example, in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety. In these embodiments, the matrix sustainedly releases the drug.

Pharmaceutically acceptable carriers and/or diluents may also include any solvents, dispersion media, coatings, antibacterials and/or antifungals, isotonic and absorption delaying agents and the like. The use of such media and agents for pharmaceutically active substances is well known in the art. Except insofar as any conventional medium or agent is incompatible with the active ingredient, use thereof in the pharmaceutical compositions is contemplated.

The pharmaceutical compositions can be formulated for administration in accordance with conventional techniques. See, e.g., Remington, The Science and Practice of Pharmacy (20th Ed. 2000). For example, the intranasal pharmaceutical compositions of the present disclosure can be formulated as an aerosol (this term includes both liquid and dry powder aerosols). Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles (e.g., lyophilized, freeze dried, etc.) can likewise be produced with any solid particulate medicament aerosol generator, by techniques known in the pharmaceutical art. As another example, the pharmaceutical compositions can be formulated as an on-demand dissolvable form, which provides a lyophilized portion of the pharmaceutical composition and a dissolving solution portion of the pharmaceutical composition.

In some embodiments, the pharmaceutical composition is in the form of an aqueous suspension, which can be prepared from solutions or suspensions. With respect to solutions or suspensions, dosage forms can be comprised of micelles of lipophilic substances, liposomes (phospholipid vesicles/membranes) and/or a fatty acid (e.g., palmitic acid). In particular embodiments, the pharmaceutical composition is a solution or suspension that is capable of dissolving in the fluid secreted by mucous membranes of the epithelium of the tissue to which it is administered, applied and/or delivered, which can advantageously enhance absorption.

The pharmaceutical composition can be an aqueous solution, a nonaqueous solution or a combination of an aqueous and nonaqueous solution. Suitable aqueous solutions include, but are not limited to, aqueous gels, aqueous suspensions, aqueous microsphere suspensions, aqueous microsphere dispersions, aqueous liposomal dispersions, aqueous micelles of liposomes, aqueous microemulsions, and any combination of the foregoing, or any other aqueous solution that can dissolve in the fluid secreted by the mucosal membranes of the nasal cavity. Exemplary nonaqueous solutions include, but are not limited to, nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing, or any other nonaqueous solution that can dissolve or mix in the fluid secreted by mucosal membranes.

Examples of powder formulations include, without limitation, simple powder mixtures, micronized powders, freeze dried powder, lyophilized powder, powder microspheres, coated powder microspheres, liposomal dispersions, and any combination of the foregoing. Powder microspheres can be formed from various polysaccharides and celluloses, which include without limitation starch, methylcellulose, xanthan gum, carboxymethylcellulose, hydroxypropyl cellulose, carbomer, alginate polyvinyl alcohol, acacia, chitosans, and any combination thereof.

In particular embodiments, the composition is one that is at least partially, or even substantially (e.g., at least 80%, 90%, 95% or more) soluble in the fluids that are secreted by mucosa so as to facilitate absorption. Alternatively or additionally, the composition can be formulated with a carrier and/or other substances that foster dissolution of the agent within secretions, including without limitation fatty acids (e.g., palmitic acid), gangliosides (e.g., GM-1), phospholipids (e.g., phosphatidylserine), and emulsifiers (e.g., polysorbate 80).

Those skilled in the art will appreciate that for intranasal administration or delivery, because the volume of the pharmaceutical composition administered is generally small, nasal secretions may alter the pH of the administered dose since the range of pH in the nasal cavity can be as wide as 5 to 8. Such alterations can affect the concentration of un-ionized drug available for absorption. Accordingly, in representative embodiments, the pharmaceutical composition further comprises a buffer to maintain or regulate pH in situ. Typical buffers include, but are not limited to, ascorbate, acetate, citrate, prolamine, carbonate, and phosphate buffers.

In embodiments, the pH of the pharmaceutical composition is selected so that the internal environment of the mucosal tissue after administration is on the acidic to neutral side, which (1) can provide the active compound in an un-ionized form for absorption, (2) prevents growth of pathogenic bacteria, which is more likely to occur in an alkaline environment, and (3) reduces the likelihood of irritation of the mucosa.

For liquid and powder sprays or aerosols, the pharmaceutical composition can be formulated to have any suitable and desired particle or droplet size. In illustrative embodiments, the majority and/or the mean size of the particles or droplets range from equal to or greater than about 1, 2.5, 5, 10, 15 or 20 microns and/or equal to or less than about 25, 30, 40, 45, 50, 60, 75, 100, 125, 150, 175, 200, 225, 250, 275, 300, 325, 350, 375, 400, or 425 microns (including all combinations of the foregoing). Representative examples of suitable ranges for the majority and/or mean particle or droplet size include, without limitation, from about 5 to 100 microns, from about 10 to 60 microns, from about 175 to 325 microns, and from about 220 to 300 microns which facilitate the deposition of a safe and effective amount of the active compound, for example, in the nasal cavity (e.g., in the upper third of the nasal cavity, the superior meatus, the olfactory region and/or the sinus region to target the olfactory neural pathway). In general, particles or droplets smaller than about 5 microns will be deposited in the trachea or even the lung, whereas particles or droplets that are about 50 microns or larger generally do not reach the nasal cavity and are deposited in the anterior nose.

International patent publication WO 2005/023335 (Kurve Technology, Inc.) describes particles and droplets having a diameter size suitable for the practice of representative embodiments of pharmaceutical compositions disclosed herein. In particular embodiments, the particles or droplets have a mean diameter of about 5 to 30 microns, about 10 to 20 microns, about 10 to 17 microns, about 10 to 15 microns, about 12 to 17 microns, about 10 to 15 microns or about 10 to 12 microns. The particles can “substantially” have a mean diameter or size as described herein, i.e., at least about 50%, 60%, 70%, 80%, 90% or 95 or more of the particles are of the indicated diameter or size range.

The pharmaceutical composition can be delivered as a nebulized or atomized liquid having a droplet size as described above.

According to particular embodiments of this disclosure that comprise methods of intranasal delivery, it can be desirable to prolong the residence time of the pharmaceutical composition in the nasal cavity (e.g., in the upper third of the nasal cavity, the superior meatus, the olfactory region and/or in the sinus region), for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl), which are agents that enhance residence time in the nasal cavity. As a further approach, increasing the viscosity of the formulation can also provide a means of prolonging contact of the agent with the nasal epithelium. The pharmaceutical composition can be formulated as a nasal emulsion, ointment or gel, which offers advantages for local application because of their viscosity.

Moist and highly vascularized membranes can facilitate rapid absorption; consequently, the pharmaceutical composition can optionally comprise a humectant, particularly in the case of a gel-based composition so as to assure adequate intranasal moisture content. Examples of suitable humectants include but are not limited to glycerin or glycerol, mineral oil, vegetable oil, membrane conditioners, soothing agents, and/or sugar alcohols (e.g., xylitol, sorbitol; and/or mannitol). The concentration of the humectant in the pharmaceutical composition will vary depending upon the agent selected and the formulation.

The pharmaceutical composition can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of about 2% by weight or less.

The pharmaceutical compositions described herein can optionally contain an odorant, e.g., as described in EP 0 504 263 B1, to provide a sensation of odor, to aid in inhalation of the composition so as to promote delivery to the olfactory region and/or to trigger transport by the olfactory neurons.

As another option, the composition can comprise a flavoring agent, e.g., to enhance the taste and/or acceptability of the composition to the subject.

### Porous Particles for Pulmonary Administration

In some embodiments, the particles are porous, so that they have an appropriate density to avoid deposition in the back of the throat when administered via an inhaler. The combination of relatively large particle size and relatively low density avoids phagocytosis in the lungs, provides appropriately targeted delivery, avoids systemic delivery of the components, and provides a high concentration of the components in the lung.

Representative methods for preparing such particles, and for delivering such particles, are described, for example, in U.S. Pat. No. 7,384,649, entitled, “Particulate compositions for pulmonary delivery,” U.S. Pat. No. 7,182,961, entitled “Particulate compositions for pulmonary delivery,” U.S. Pat. No. 7,146,978, entitled, “Inhalation device and method,” U.S. Pat. No. 7,048,908, entitled “Particles for inhalation having sustained release properties,” U.S. Pat. No. 6,956,021, entitled “Stable spray-dried protein formulations,” U.S. Pat. No. 6,766,799, entitled “Inhalation device,” and U.S. Pat. No. 6,732,732, entitled “Inhalation device and method.”

Additional patents disclosing such particles include U.S. Pat. No. 7,279,182, entitled “Formulation for spray-drying large porous particles,” U.S. Pat. No. 7,252,840, entitled “Use of simple amino acids to form porous particles,” U.S. Pat. No. 7,032,593, entitled “Inhalation device and method,” U.S. Pat. No. 7,008,644, entitled “Method and apparatus for producing dry particles,” U.S. Pat. No. 6,848,197, entitled “Control of process humidity to produce large, porous particles,” and U.S. Pat. No. 6,749,835, entitled “Formulation for spray-drying large porous particles.”

U.S. Pat. No. 7,678,364, entitled “Particles for inhalation having sustained release properties,” discloses methods for delivering particles to the pulmonary system comprising: administering to the respiratory tract of a patient in need of treatment, prophylaxis or diagnosis a safe and effective amount of a dry powder comprising: a) a multivalent metal cation which is complexed with a therapeutic, prophylactic or diagnostic agent; b) a pharmaceutically acceptable carrier; and c) a multivalent metal cation-containing component wherein the dry powder is spray-dried and has a total amount of multivalent metal cation which is about 10% w/w or more of the total weight of the agent, a tap density of about 0.4 g/cm³ or less, a median geometric diameter of from about 5 micrometers to about 30 micrometers and an aerodynamic diameter of from about 1 to about 5 microns.

The amount of the compounds described herein, or salts thereof, present in the particles can range from about 0.1 weight % to about 95 weight %, though in some cases, can even be as high as 100%. For example, from about 1 to about 50%, such as from about 5 to about 30%. Particles in which the compound is distributed throughout a particle can be preferred.

In some embodiments, the particles include a surfactant other than the phospholipids described above. As used herein, the term “surfactant” refers to any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to particles, they tend to present moieties to the external environment that do not attract similarly-coated particles, thus reducing particle agglomeration. Surfactants may also promote absorption of a therapeutic or diagnostic agent and increase bioavailability of the agent.

Suitable surfactants which can be employed in fabricating the particles disclosed herein include but are not limited to hexadecanol; fatty alcohols such as polyethylene glycol (PEG); polyoxyethylene-9-lauryl ether; a surface active fatty acid, such as palmitic acid or oleic acid; glycocholate; surfactin; a poloxamer; a sorbitan fatty acid ester such as sorbitan trioleate (Span 85); Tween® 80 and tyloxapol.

The surfactant can be present in the particles in an amount ranging from about 0 to about 5 weight %. Preferably, it can be present in the particles in an amount ranging from about 0.1 to about 1.0 weight %.

Particles that have a tap density less than about 0.4 g/cm³, median diameters of at least about 5 μm, and an aerodynamic diameter of from about 1 μm to about 5 μm, or from about 1 μm to about 3 μm, are more capable of escaping inertial and gravitational deposition in the oropharyngeal region, and are targeted to the airways or the deep lung. The use of larger, more porous particles is advantageous since they are able to aerosolize more efficiently than smaller, denser aerosol particles such as those currently used for inhalation therapies.

### Liposomal Delivery

The compositions described herein are advantageously delivered to the lungs, so as to provide the compounds at the site of an actual or potential norovirus or coronavirus infection. This can be accomplished by pulmonary delivery via metered-dose inhalers or other pulmonary delivery devices, and also by lodging particles in the capillary beds surrounding the alveoli in the lungs.

Nanocarriers, such as liposomes, including small unilamellar vesicles, show several advantages over other conventional approaches for delivering drugs to the lungs, including prolonged drug release and cell-specific targeted drug delivery. Nano-sized drug carriers can also be advantageous for delivering poorly water soluble drugs, and certain of the compounds described herein are poorly water-soluble. Additional advantages include their ability to provide controlled release, protection from metabolism and degradation, decreased drug toxicity and targeting capabilities.

The liposomes (preferably unilamellar vesicles) have a size less than 200 nm as measured by dynamic light scattering, and preferably characterized by being comprised of chemically pure synthetic phospholipids, most preferably having aliphatic side chains of a length of at least 16 carbons, and containing one or more of the compounds described herein, or a pharmaceutically acceptable salt thereof, sufficient to preferentially deliver (i.e., target) a quantity of the compounds thereof to the capillary beds surrounding the alveoli. Vesicle diameter can be measured, for example, by dynamic light scattering using a helium-neon 100 mW NEC gas laser and a Malvern K7027 correlator, ideally with at least two or three measurements made for each for each size determination.

The expression “chemically pure phospholipids” is meant to define phospholipids which are essentially free of deleterious detergent moieties and impurities which cause aggregation of small unilamellar vesicles (SUVs) formed therefrom, and which are more than 97% pure. Preferably, the liposomes have a diameter predominantly of from about 50 to about 160 nm, are essentially neutral in charge, and incorporate phospholipids having a side chain length of from 16 to 18 carbon atoms. More preferably, the liposomes are prepared from distearoyl phosphatidylcholine (DSPC) and include cholesterol (most preferably in an amount of from 10 to 50% of total lipid) as a vesicle stabilizer.

It can also be advantageous that the liposomes have a melting point above body temperature (i.e., above 37° C.). For this reason, it can be advantageous to use pure phospholipids, preferably ones that are saturated, and have a carbon chain length of at least 16 carbons, preferably between 16 and 18 carbons. Distearoylphosphatidyl choline (DSPC) is a preferred phospholipid.

Cholesterol helps to stabilize the liposomes, and is preferably added in a sufficient amount to provide liposome stability. Most preferably, the liposomes further comprise a pegylated phospholipid, such as DSPEPEG. The method involves introducing into a patient's bloodstream an amount of liposomes, of a size of less than 200 nm (preferably unilamellar vesicles) and preferably characterized by being comprised of chemically pure synthetic phospholipids, most preferably having aliphatic side chains of a length of at least 16 carbons, and containing the compounds described herein, or a pharmaceutically acceptable salt or prodrug thereof, sufficient to preferentially deliver (i.e., target) a quantity of the compounds to the capillary beds in the lungs, surrounding the alveoli.

The compounds described herein can be combined with other anti-norovirus or anti-coronavirus agents. Such additional agents can also be present in the liposomes, can be present in different liposomes, or can be co-administered via a different route.

The liposomes include one or more of the compounds described herein, or a pharmaceutically acceptable salt thereof, and can optionally include other anti-norovirus or anti-coronavirus agents. The liposomes can be prepared by dissolving the phospholipid and cholesterol in an appropriate organic solvent, such as chloroform, and evaporating the solvent to form a lipid film. If an ionophore is employed to load the compounds described herein into the liposomes, the ionophore may be added to the lipid solution before evaporation. The dried lipid film is then rehydrated in an appropriate aqueous phase, such as phosphate-buffered saline or other physiologically appropriate solution. Water-soluble drugs or therapeutic agents may be contained in the hydrating solution, although if remote loading is desired a loading agent such as a chelating agent described above may be added to the hydrating solution to be encapsulated within the inner aqueous space of the liposome.

Upon the addition of the hydrating solution, liposomes of varying size spontaneously form and encapsulate a portion of the aqueous phase. Thereafter, the liposomes and suspending aqueous solution are subjected to a shear force such as extrusion, sonication, or processing through a homogenizer according to the method described in U.S. Pat. No. 4,753,788; to produce vesicles within the specified size.

The liposomes can then be processed to remove undesirable compounds from the suspending solution, for example, un-encapsulated drug, which may be accomplished through processes such as gel chromatography or ultrafiltration.

The use of liposomes in dry powder aerosols for targeted lung delivery is described, for example, in Willis et al., Lung, June 2012, 190(3):251-262. One advantage is that the phospholipids used to prepare the liposomes are similar to endogenous lung surfactant.

### Routes of Administration and Dosages

The compounds and pharmaceutically acceptable compositions described above can be administered to humans and other animals orally, rectally, parenterally, intracisternally, intravaginally, intraperitoneally, topically (as by powders, ointments, or drops), bucally, as an oral or nasal spray, to the pulmonary system, such as by using an inhaler, such as a metered dose inhaler (MDI), or the like, depending on the severity of the infection being treated. In some embodiments, the compound or composition disclosed herein is administered orally, via inhalation, or intravenously.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topical application also includes the use of transdermal patches.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

### Methods of Treatment

Provided herein are uses of a compound described herein as a therapeutic agent. The compounds described herein or pharmaceutically acceptable salts thereof can be used to reduce viral titer in a biological sample (e.g., an infected cell culture) or in humans (e.g. lung viral titer in a patient). The compounds described herein or pharmaceutically acceptable salts thereof can be used in methods of treating viral infections. Non-limiting examples of viral infections which can be treated with the compounds described herein or their pharmaceutically acceptable salts include coronavirus infections, calicivirus infections, and picornavirus infections.

Non-limiting examples of calicivirus infections include norovirus mediated conditions and norovirus infection. The terms “norovirus mediated condition”, “norovirus infection”, and “norovirus”, as used herein, are used interchangeably to mean the disease caused by an infection with a norovirus.

Noroviruses are infectious viruses that cause gastroenteritis in mammals. Noroviruses are RNA viruses of the family Caliciviridae, which comprises seven genogroups: GI, GII, GIII, GIV, GV, GVI, and GVII. Genogroup II, the most prevalent human genogroup, presently contains 19 genotypes. Genogroups I, I and IV infect humans, whereas genogroup III infects bovine species, and genogroup V has recently been isolated in mice. The two groups most associated with gastroenteritis in humans are genogroup I (GI), which includes Norwalk virus, Desert Shield virus and Southampton virus; and genogroup II (GII), which includes Bristol virus, Lordsdale virus, Toronto virus, Mexico virus, Hawaii virus and Snow Mountain virus.

In some embodiments, the compounds used herein are for treatment of noroviruses which are associated with gastroenteritis. In some embodiments, noroviruses are associated with Norwalk virus. In some embodiments, noroviruses are associated with HuNV GGII.4.

In some embodiments, the compounds disclosed herein can be used in the treatment of norovirus, wherein the compound binds to free virus, or inhibits a norovirus protease. In some cases, the compound can target both (free virus and protease).

In humans, common symptoms of norovirus are nausea, vomiting, watery diarrhea, abdominal pain, and in some cases, loss of taste. Norovirus can establish a long term infection in people who are immunocompromised. In severe cases, persistent infections can lead to norovirus-associated enteropathy, intestinal villous atrophy, and malabsorption. Norovirus-associated gastroenteritis is also called “winter vomiting bug”.

A person usually develops symptoms of gastroenteritis 12 to 48 hours after being exposed to norovirus. General lethargy, weakness, muscle aches, headaches, and low-grade fevers may occur.

The term “coronavirus infection” as used herein means the disease caused by an infection with a coronavirus. Non-limiting examples of coronaviruses include severe acute respiratory syndrome-related coronavirus (SARS), Middle East respiratory syndrome-related coronavirus (MERS), and SARS-CoV-2 virus (also known as 2019-nCoV, or Wuhan coronavirus). Non-limiting examples of coronavirus infections include SARS, MERS, and COVID-19 (i.e., SARS-CoV infection, MERS-CoV infection, and SARS-CoV-2 infection, respectively).

Coronaviruses are a family of viruses that cause diseases in mammals and birds. Coronaviruses are in the subfamily Orthocoronavirinae in the family Coronaviridae, in the order Nidovirales. There are four main genera of coronaviruses, known as alpha, beta, gamma, and delta. Coronaviruses that affect humans include Human coronavirus 229E (HCoV-229E), Human coronavirus OC43 (HCoV—OC43), Severe acute respiratory syndrome-related coronavirus (SARS-CoV), Human coronavirus NL63 (HCoV-NL63, New Haven coronavirus), Human coronavirus HKU1, Middle East respiratory syndrome-related coronavirus (MERS-CoV, previously known as novel coronavirus 2012 and HCoV-EMC), and SARS-CoV-2 (also known as 2019-nCoV and Wuhan coronavirus).

In humans, coronaviruses cause respiratory infections, including the common cold, which are typically mild, though rarer forms such as SARS, MERS and SARS-CoV-2 (the cause of the 2019-20 COVID-19 outbreak) can be lethal. Symptoms vary in other species: in chickens, they cause an upper respiratory disease, while in cows and pigs coronaviruses cause diarrhea. There are no vaccines or antiviral drugs to prevent or treat human coronavirus infections. The coronaviruses HCoV-229E, -NL63, —OC43, and —HKU1 continually circulate in the human population and cause respiratory infections in adults and children worldwide

In some embodiments, the compounds used herein are for treatment of alphacoronaviruses or betacoronaviruses. In some cases, the compounds used herein are for treatment of alphacoronaviruses. Non-limiting examples of alphacoronaviruses include HCoV-229E and HCoV-NL63. In some embodiments, the compounds used herein are for treatment of betacoronaviruses. Non-limiting examples of betacoronaviruses are HCoV—HKU1, HCoV—OC43, Middle East respiratory syndrome coronavirus (MERS-CoV), the severe acute respiratory syndrome coronavirus (SARS-CoV), and SARS-CoV-2. In some embodiments, the compounds used herein are for treatment of coronaviruses which are associated with SARS, MERS, and COVID-19. In some embodiments, coronaviruses are associated with SARS. In some embodiments, coronaviruses are associated with MERS. In some embodiments, coronaviruses are associated with COVID-19.

In some embodiments, the compounds disclosed herein can be used in the treatment of coronavirus, wherein the compound binds to free virus, or inhibits a coronavirus protease. In some cases, the compound can target both (free virus and protease).

In humans, common symptoms of coronavirus are fever, cough, shortness of breath, and myalgia.

Non-limiting examples of picornavirus infections include rhinovirus mediated conditions and rhinovirus infections. The terms “rhinovirus mediated condition” and “rhinovirus infection” as used herein, are used interchangeably to mean the disease caused by an infection with a rhinovirus.

Picornaviruses infect both humans and animals, can cause severe paralysis (paralytic poliomyelitis), aseptic meningitis, hepatitis, pleurodynia, myocarditis, skin rashes, and colds; although asymptomatic infection is common. Several medically important genera are members of this family, such as enterovirus (including poliovirus (PV), rhinoviruses, and human enteroviruses (e.g. coxsackie viruses)); hepatovirus which includes hepatitis A virus (HAV); and aphthoviruses which include the foot- and mouth disease virus (FMDV). Rhinoviruses are recognized as the principle cause of the common cold in humans, and comprise three different species: A, B, and C. Transmission is primarily by the aerosol route and the virus replicates in the nose.

In some embodiments, the compounds disclosed herein can be used in the treatment of picornavirus infection. In some embodiments, the compounds disclosed herein can be used in the treatment of rhinovirus infection.

In some embodiments, the compounds disclosed herein can be used in the treatment of rhinovirus infection wherein the compound binds to free virus, or inhibits a rhinovirus protease. In some cases, the compound can target both (free virus and protease).

The term “disease” as used herein refers to a coronavirus infection-related medical or pathological condition.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

The term “biological sample”, as used herein, includes, without limitation, cell cultures or extracts thereof; biopsied material obtained from a subject or extracts thereof; blood, saliva, urine, feces, semen, tears, or other body fluids or extracts thereof.

As used herein, “multiplicity of infection” or “MOI” is the ratio of infectious agents (e.g. phage or virus) to infection targets (e.g. cell). For example, when referring to a group of cells inoculated with infectious virus particles, the multiplicity of infection or MOI is the ratio defined by the number of infectious virus particles deposited in a well divided by the number of target cells present in that well.

As used herein the terms “inhibition of the replication of noroviruses” and “inhibition of the replication of coronaviruses” includes the reduction in the amount of virus replication (e.g., the reduction by at least 10%), which may be sufficient to result in the complete arrest of virus replication (i.e., 100% reduction). In some embodiments, the replication of norovirus or coronavirus viruses are inhibited by at least 20%, at least 30%, at least 40%, at least 50%, at least 65%, at least 75%, at least 85%, at least 90%, or at least 95%.

Norovirus or coronavirus virus replication can be measured by any suitable method known in the art. For example, norovirus or coronavirus viral titer in a biological sample (e.g. an infected cell culture) or in humans (e.g. lung viral titer in a patient) can be measured. More specifically, for cell based assays, in each case cells are cultured in vitro, virus is added to the culture in the presence or absence of a test agent, and after a suitable length of time a virus-dependent endpoint is evaluated. Such assays are known in the art. A first type of cell assay that can be used in the disclosure depends on death of the infected target cells, a process called cytopathic effect (CPE), where virus infection causes exhaustion of the cell resources and eventual lysis of the cell. In the first type of cell assay, a low fraction of cells in the wells of a microtiter plate are infected (typically 1/10 to 1/1000), the virus is allowed to go through several rounds of replication over 48-72 hours, then the amount of cell death is measured using a decrease in cellular ATP content compared to uninfected controls. A second type of cell assay that can be employed in the disclosure depends on the multiplication of virus-specific RNA molecules in the infected cells, with RNA levels being directly measured using the branched-chain DNA hybridization method (bDNA). In the second type of cell assay, a low number of cells are initially infected in wells of a microtiter plate, the virus is allowed to replicate in the infected cells and spread to additional rounds of cells, then the cells are lysed and viral RNA content is measured. This assay is stopped early, usually after 18-36 hours, while all the target cells are still viable. Viral RNA is quantitated by hybridization to specific oligonucleotide probes fixed to wells of an assay plate, then amplification of the signal by hybridization with additional probes linked to a reporter enzyme.

As used herein a “viral titer (or titer)” is a measure of virus concentration. Titer testing can employ serial dilution to obtain approximate quantitative information from an analytical procedure that inherently only evaluates as positive or negative. The titer corresponds to the highest dilution factor that still yields a positive reading; for example, positive readings in the first 8 serial twofold dilutions translate into a titer of 1:256. To determine the titer, several dilutions will be prepared, such as 10⁻¹, 10⁻², 10⁻³, 10⁻⁸.

As used herein, the terms “treat”, “treatment” and “treating” refer to both therapeutic and prophylactic treatments. For example, therapeutic treatments include the reduction or mitigation of the progression, severity and/or duration of norovirus or coronavirus infections, or the amelioration of one or more symptoms (specifically, one or more discernible symptoms) of norovirus or coronavirus infections, resulting from the administration of one or more therapies (e.g., one or more therapeutic agents such as a compound or composition of the disclosure). In specific embodiments, the therapeutic treatment includes the amelioration of at least one measurable physical parameter of a norovirus or coronavirus infection. In other embodiments the therapeutic treatment includes the inhibition of the progression of a norovirus or coronavirus infection, either physically by, e.g., stabilization of a discernible symptom, physiologically by, e.g., stabilization of a physical parameter, or both. In other embodiments the therapeutic treatment includes the reduction or stabilization of norovirus or coronavirus mediated infections. Antiviral drugs can be used in the community setting to treat people who already have norovirus or coronavirus to reduce the severity of symptoms and reduce the number of days that they are sick.

The term “chemotherapy” refers to the use of medications, e.g. small molecule drugs (rather than “vaccines”) for treating a disorder or disease.

The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease. The term “chemoprophylaxis” refers to the use of medications, e.g. small molecule drugs (rather than “vaccines”) for the prevention of a disorder or disease.

As used herein, prophylactic use includes the use in situations in which an outbreak has been detected, to prevent contagion or spread of the infection in places where a lot of people that are at high risk of serious norovirus or coronavirus complications live in close contact with each other (e.g. in a hospital ward, daycare center, prison, nursing home, etc.). It also includes the use among populations who require protection from the norovirus or coronavirus but who either do not get protection after vaccination (e.g. due to weak immune system), or when the vaccine is unavailable to them, or when they cannot get the vaccine because of side effects. It also includes use during the two weeks following vaccination, since during that time the vaccine is still ineffective. Prophylactic use may also include treating a person who is not ill with the norovirus or coronavirus or not considered at high risk for complications, in order to reduce the chances of getting infected with norovirus or coronavirus and passing it on to a high-risk person in close contact with him (for instance, healthcare workers, nursing home workers, etc.).

In some embodiments, the methods of the disclosure are a preventative or “prophylactic” measure to a patient, specifically a human, having a predisposition to complications resulting from infection by a norovirus or coronavirus virus. Prophylactic use includes use in situations in which an “index case” or an “outbreak” has been confirmed, in order to prevent the spread of infection in the rest of the community or population group.

In embodiments, the methods of the disclosure are applied as a “prophylactic” measure to members of a community or population group, specifically humans, in order to prevent the spread of infection.

As used herein, an “effective amount” refers to an amount sufficient to elicit the desired biological response.

In the present disclosure the desired biological response is to inhibit the replication of noroviruses or coronaviruses, to reduce the amount of noroviruses or coronaviruses or to reduce or ameliorate the severity, duration, progression, or onset of a norovirus or coronavirus infection, prevent the advancement of a norovirus or coronavirus infection, prevent the recurrence, development, onset or progression of a symptom associated with a norovirus or coronavirus infection, or enhance or improve the prophylactic or therapeutic effect(s) of another therapy used against norovirus or coronavirus infections. The amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other anti-viral agents, e.g., when co-administered with an anti-norovirus or anti-coronavirus medication, an “effective amount” of the second agent will depend on the type of agent used. Suitable dosages are known for approved agents and can be adjusted by the skilled artisan according to the condition of the subject, the type of disease(s) being treated and the amount of a compound described herein being used. In cases where no amount is expressly noted, a safe and effective amount should be assumed. For example, compounds described herein can be administered to a subject in a dosage range from between approximately 0.01 to 100 mg/kg body weight/day for therapeutic or prophylactic treatment.

Generally, dosage regimens can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe the effective amount of the compounds described herein required to treat, to prevent, inhibit (fully or partially) or arrest the progress of the disease.

Dosages of the compounds (e.g., a compound of Formula (I), Formula (Ia), Formula (Ib), or a salt thereof) for use in the methods and compositions described herein can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. In some cases, the method or composition comprises a compound of Formula (I), Formula (Ia), Formula (Ib), or a salt thereof at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In some cases, the method or composition comprises a compound of Formula (I), Formula (Ia), Formula (Ib), or a salt thereof at a dose of about 5 mg/kg. In some cases, the method or composition comprises a compound of Formula (I), Formula (Ia), Formula (Ib), or a salt thereof at a dose of about 10 mg/kg.

For therapeutic treatment, the compounds described herein can be administered to a patient within, for example, 48 hours (or within 40 hours, or less than 2 days, or less than 1.5 days, or within 24 hours) of onset of symptoms (e.g., nasal congestion, sore throat, cough, aches, fatigue, headaches, and chills/sweats). The therapeutic treatment can last for any suitable duration, for example, for 5 days, 7 days, 10 days, 14 days, etc. For prophylactic treatment during a community outbreak, the compounds described herein can be administered to a patient within, for example, 2 days of onset of symptoms in the index case, and can be continued for any suitable duration, for example, for 7 days, 10 days, 14 days, 20 days, 28 days, 35 days, 42 days, etc.

### Combination Therapy

The compounds described herein can be used in combination therapy, i.e., in conjunction with other anti-norovirus or anti-coronavirus compounds, or in conjunction with a vaccine. Combination therapy can be particularly advantageous where a patient might be exposed to more than one form of the norovirus or coronavirus virus.

A safe and effective amount can be achieved in the method or pharmaceutical composition of the disclosure employing a compound disclosed herein, or a pharmaceutically acceptable salt thereof alone or in combination with an additional suitable therapeutic agent, for example, an antiviral agent or a vaccine. When “combination therapy” is employed, a safe and effective amount can be achieved using a first amount of a compound disclosed herein, or a pharmaceutically acceptable salt thereof, and a second amount of an additional suitable therapeutic agent (e.g. an antiviral agent or vaccine).

In embodiments, the compound disclosed herein, or a pharmaceutically acceptable salt, and the additional therapeutic agent, are each administered in a safe and effective amount (i.e., each in an amount which would be therapeutically effective if administered alone). In other embodiments, the compound disclosed herein, or a pharmaceutically acceptable salt thereof, and the additional therapeutic agent, are each administered in an amount which alone does not provide a therapeutic effect (a sub-therapeutic dose). In yet other embodiments, the compound disclosed herein, or a pharmaceutically acceptable salt thereof can be administered in a safe and effective amount, while the additional therapeutic agent is administered in a sub-therapeutic dose. In still other embodiments, the compound disclosed herein, a pharmaceutically acceptable salt thereof can be administered in a sub-therapeutic dose, while the additional therapeutic agent, for example, a suitable antiviral therapeutic agent is administered in a safe and effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

In embodiments, the present disclosure is directed to methods of combination therapy for inhibiting the virus's replication in biological samples or patients, or for treating or preventing norovirus or coronavirus infections in patients using the compounds or pharmaceutical compositions described herein, e.g., a compound disclosed herein, or a pharmaceutically acceptable salt thereof. Accordingly, pharmaceutical compositions also include those comprising a compound as disclosed herein (e.g., an inhibitor of virus replication) in combination with an anti-viral compound exhibiting anti-Norovirus or coronavirus virus activity.

Methods of use of the compounds and compositions disclosed herein also include combination of chemotherapy with a compound or composition disclosed herein, or a pharmaceutically acceptable salt thereof or with a combination of a compound or composition of this disclosure with another anti-viral agent.

When co-administration involves the separate administration of the first amount of a compound disclosed herein, or a pharmaceutically acceptable salt thereof and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound disclosed herein, or a pharmaceutically acceptable salt thereof and the second therapeutic agent can be administered in any order within about 24 hours of each other, within about 16 hours of each other, within about 8 hours of each other, within about 4 hours of each other, within about 1 hour of each other or within about 30 minutes of each other.

More specifically, a first therapy (e.g., a prophylactic or therapeutic agent such as a compound of the disclosure) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-viral agent) to a subject.

It is understood that the method of co-administration of a first amount of a compound disclosed herein, or a pharmaceutically acceptable salt thereof and a second amount of an additional therapeutic agent can result in an enhanced or synergistic therapeutic effect, wherein the combined effect is greater than the additive effect that would result from separate administration of the first amount of the compound disclosed herein, or a pharmaceutically acceptable salt thereof and the second amount of the additional therapeutic agent.

As used herein, the term “synergistic” refers to a combination of a compound disclosed herein and another therapy (e.g., a prophylactic or therapeutic agent), which is more effective than presumed additive effects of the therapies. A synergistic effect of a combination of therapies (e.g., a combination of prophylactic or therapeutic agents) can permit the use of lower dosages of one or more of the therapies and/or less frequent administration of said therapies to a subject. The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

When the combination therapy using compounds as disclosed herein is in combination with a virus vaccine, both therapeutic agents can be administered so that the period of time between each administration can be longer (e.g. days, weeks or months).

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S, and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

### Chiral Separations

The compounds described herein can have asymmetric centers and occur as racemates, racemic mixtures, individual diastereomers or enantiomers, with all isomeric forms being included in the present disclosure. Compounds of the present disclosure having a chiral center can exist in and be isolated in optically active and racemic forms. Some compounds can exhibit polymorphism. The present disclosure encompasses racemic, optically-active, polymorphic, or stereoisomeric forms, or mixtures thereof, of a compound of the disclosure, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.
i) Physical separation of crystals: a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization: a technique whereby the individual enantiomers are separately crystallized from a solution of the racemate, possible only if the latter is a conglomerate in the solid state;
iii) enzymatic resolutions: a technique whereby partial or complete separation of a racemate by virtue of differing rates of reaction for the enantiomers with an enzyme;
iv) enzymatic asymmetric synthesis: a synthetic technique whereby at least one step of the synthesis uses an enzymatic reaction to obtain an enantiomerically pure or enriched synthetic precursor of the desired enantiomer;
v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;
vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors: a synthetic technique whereby the desired enantiomer is obtained from non-chiral starting materials and where the stereochemical integrity is not or is only minimally compromised over the course of the synthesis;
x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;
xi) chiral gas chromatography: a technique whereby the racemate is volatilized and enantiomers are separated by virtue of their differing interactions in the gaseous mobile phase with a column containing a fixed non-racemic chiral adsorbent phase;
xii) extraction with chiral solvents: a technique whereby the enantiomers are separated by virtue of preferential dissolution of one enantiomer into a particular chiral solvent;
xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

The present disclosure will be better understood with reference to the following non-limiting examples.

### Compound Synthesis

### Example 1: Synthesis of Int-7

### Preparation of Int-7

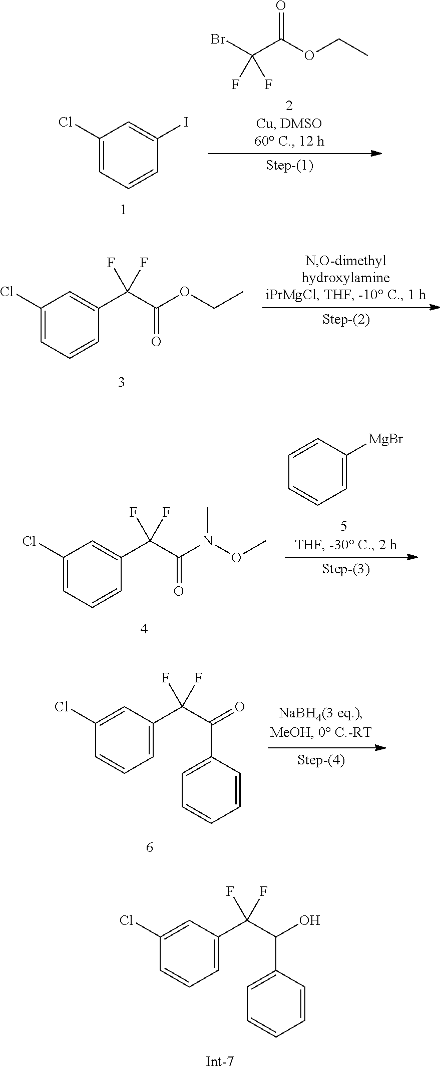

### Ethyl 2-(3-chlorophenyl)-2,2-difluoroacetate (3)

Ethyl 2-bromo-2,2-difluoroacetate (2) (16.9 g, 84.03 mmol) was added to a suspension of copper powder (10.58 g, 168.06 mmol) in DMSO (100 mL) under N₂, and the reaction was stirred for 1 hour at room temperature, then 1-chloro-3-iodobenzene (10 g, 42.016 mmol) was added, and the reaction was stirred at 60° C. for 16 hours. The progress of the reaction was monitored by TLC. The reaction mixture was quenched with aqueous NH₄Cl solution (200 mL), and extracted with diethyl ether (2×150 mL), the combined organic layers were washed with water (250 ml) and brine solution (300 mL), dried over sodium sulfate and evaporated. The crude residue was purified by normal phase chromatography to afford ethyl 2-(3-chlorophenyl)-2,2-difluoroacetate (3). TLC system: 5% Ethyl acetate/Pet ether R_{f}: 0.2

### 2-(3-Chlorophenyl)-2,2-difluoro-N-methoxy-N-methylacetamide (4)

To a stirred solution of ethyl 2-(3-chlorophenyl)-2,2-difluoroacetate (3) (1 g, 4.273 mmol) in THF (20 mL) was added N,O-dimethyl hydroxylamine. hydrochloride (0.62 g, 6.41 mmol), and the reaction was cooled to −10° C., then isopropyl magnesium chloride 1.0 M in THF (12.8 mL, 12.82 mmol) was added slowly and stirred for 2 hours at same temperature. The progress of the reaction was monitored by TLC. The reaction mixture was quenched with saturated ammonium chloride solution (50 mL) and extracted with ethyl acetate (2×50 mL). The combined organic layers were washed with water (120 mL), followed by brine solution (150 mL), then dried over sodium sulfate and evaporated under reduced pressure to afford 2-(3-chlorophenyl)-2,2-difluoro-N-methoxy-N-methylacetamide (4). TLC system: 20% Ethyl acetate/Pet ether R_{f}: 0.2. LCMS (ESI): m/z 250.10 [M+Na]⁺

### 2-(3-Chlorophenyl)-2,2-difluoro-1-phenylethan-1-one (6)

To a stirred solution of 2-(3-chlorophenyl)-2,2-difluoro-N-methoxy-N-methylacetamide (4) (5.5 g, 22.08 mmol) in THE (50 mL) was added phenylmagnesium bromide (5) (44 mL, 44.17 mmol) at −30° C. and the mixture was stirred at room temperature for 3 hours. Reaction progress was monitored by TLC. The reaction mixture was quenched with saturated ammonium chloride and extracted with ethyl acetate (2×100 mL). The organic layers were combined and washed with water (150 mL) and brine solution (150 mL), then dried over sodium sulfate and evaporated under reduced pressure to afford 2-(3-chlorophenyl)-2,2-difluoro-1-phenylethan-1-one (6). TLC system: 20% Ethyl acetate in hexane R_{f}: 0.5

### 2-(3-Chlorophenyl)-2,2-difluoro-1-phenylethan-1-ol (Int-7)

To a stirred solution of 2-(3-chlorophenyl)-2,2-difluoro-1-phenylethan-1-one (6) (6 g, 22.55 mmol) in MeOH (60 mL) was added sodium borohydride (2.57 g, 67.66 mmol) at 0° C. which was stirred at room temperature for 2 hours. Reaction progress was monitored by TLC. The reaction mixture was quenched with ice water and the excess methanol was evaporated under reduced pressure. To this was added 1N HCl (150 mL), and the mixture was extracted with ethyl acetate (2×100 mL). The combined organic layers were washed with water (150 mL) and brine solution (150 mL), and dried over sodium sulfate, then evaporated under reduced pressure to afford 2-(3-chlorophenyl)-2,2-difluoro-1-phenylethan-1-ol (6). TLC system: 20% Ethyl acetate in hexane R_{f}: 0.45

### Example 2: Synthesis of Compound A20 Diastereomer 1 and 2

### A20 Diastereomer 1 (A20-1)

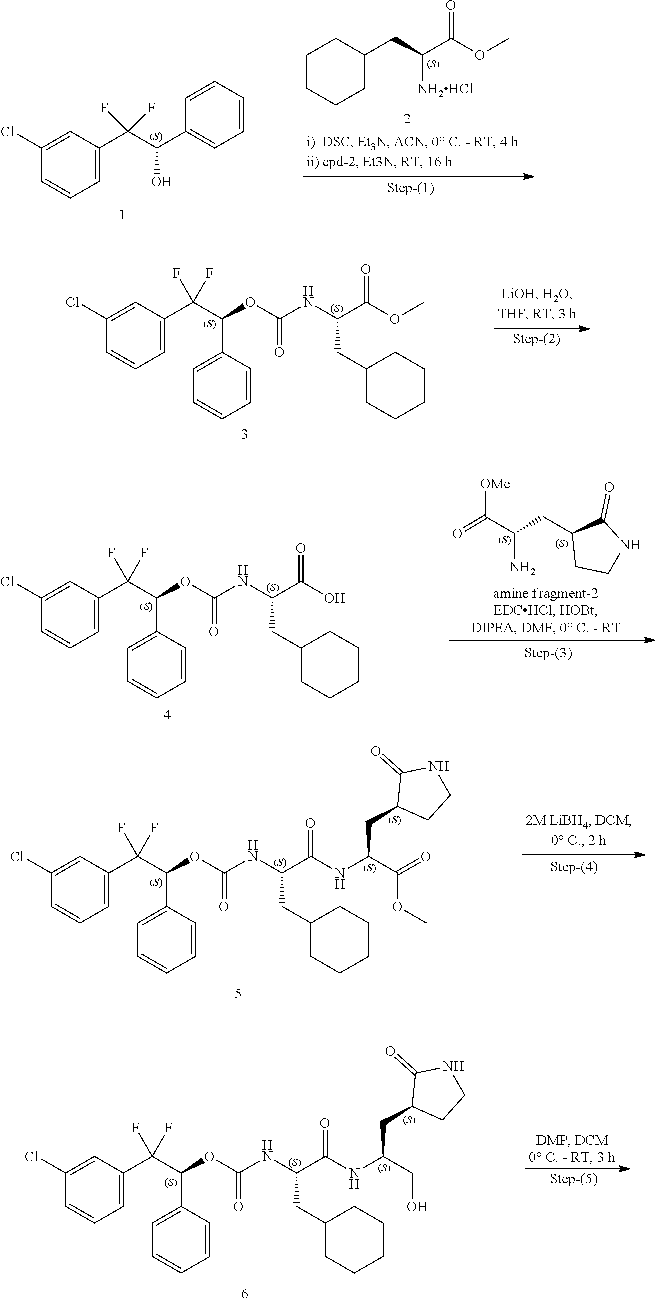

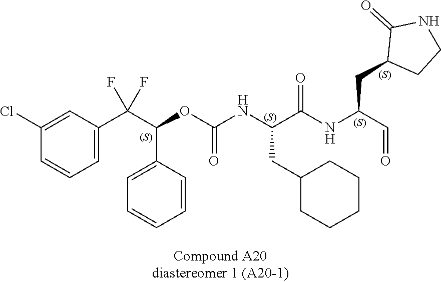

### (S)-2-(3-chlorophenyl)-2,2-difluoro-1-phenylethan-1-ol and (R)-2-(3-chlorophenyl)-2,2-difluoro-1-phenylethan-1-al

Compound (Int-7) was purified by SFC to afford (S)-2-(3-chlorophenyl)-2,2-difluoro-1-phenylethan-1-ol (Int-PK-1) and (R)-2-(3-chlorophenyl)-2,2-difluoro-1-phenylethan-1-ol (Int-PK-2). TLC system: 10% Ethyl acetate in pet ether Rf: 0.5; LCMS (ESI): m/z 558.57 [M+H]⁺

### Methyl (S)-2-((((S)-2-(3-chlorophenyl)-2,2-difluoro-1-phenylethoxy)carbonyl)amino)-3-cyclohexylpropanoate (3)

To a stirred solution of (S)-2-(3-chlorophenyl)-2,2-difluoro-1-phenylethan-1-ol (1) (3.5 g, 13.059 mmol) in ACN (35 mL) was added N, N′-disuccinamidyl carbonate (8.35 g, 32.64 mmol), followed by triethylamine (5.4 mL, 39.177 mmol) at 0° C., and the reaction mixture was stirred at room temperature for 4 h. The progress of the reaction was monitored by TLC. The reaction mass was used directly in the subsequent reaction.

In another RB flask, methyl (S)-2-amino-3-cyclohexylpropanoate hydrochloride (2) (5.77 g, 26.118 mmol) was taken in ACN (35 mL), and treated with triethylamine (5.4 mL, 39.177 mmol mmol). The resulting reaction mixture was stirred for 5 min, then added to the above prepared reaction mass drop-wise and the reaction mixture was stirred at room temperature for 16 hours. The reaction mixture was quenched with ice water (150 mL) and extracted with ethyl acetate (2×100 mL). The combined organic layers were washed with brine solution (100 mL), dried over sodium sulfate and evaporated under reduced pressure. The residue was purified by reverse phase chromatography to afford methyl (S)-2-((((S)-2-(3-chlorophenyl)-2,2-difluoro-1-phenylethoxy)carbonyl)amino)-3-cyclohexylpropanoate (3). TLC system: 10% Ethyl acetate in hexane Rf: 0.55; LCMS (ESI): m/z 502.47 [M+Na]⁺

### (S)-2-((((S)-2-(3-chlorophenyl)-2,2-difluoro-1-phenylethoxy)carbonyl)amino)-3-cyclohexylpropanoic acid (4)

To a stirred solution of methyl (S)-2-((((S)-2-(3-chlorophenyl)-2,2-difluoro-1-phenylethoxy)carbonyl)amino)-3-cyclohexylpropanoate (3) (4 g, 8.35 mmol) in THE (40 mL), water (20 mL) was added lithium hydroxide (0.7 g, 16.701 mmol) at room temperature and the mixture was stirred at room temperature for 3 hours. The progress of the reaction was monitored by TLC and LCMS. The reaction mixture was completely distilled under reduced pressure, the residue was acidified with aqueous 1N HCl solution up to pH˜2, then extracted with dichloromethane (2×100 mL), dried over sodium sulfate, and concentrated under reduced pressure to afford (S)-2-((((S)-2-(3-chlorophenyl)-2,2-difluoro-1-phenylethoxy)carbonyl)amino)-3-cyclohexylpropanoic acid (4). TLC system: 50% Ethyl acetate in hexane Rf: 0.1; LCMS (ESI): m/z=953.64 [2M+Na]⁺

### Methyl (S)-2-((S)-2-((((S)-2-(3-chlorophenyl)-2,2-difluoro-1-phenylethoxy)carbonyl)amino)-3-cyclohexylpropanamido)-3-((S)-2-oxopyrrolidin-3-yl)propanoate (5)

To a stirred solution of (S)-2-((((S)-2-(3-chlorophenyl)-2,2-difluoro-1-phenylethoxy)carbonyl)amino)-3-cyclohexylpropanoic acid (4) (1.5 g, 3.225 mmol) in DMF (15 mL) was added EDC-HCl (0.923 g, 4.8375 mmol), HOBt (0.65 g, 4.8375 mmol), DIPEA (1.65 mL, 35.481 mmol) and methyl (S)-2-amino-3-((S)-2-oxopyrrolidin-3-yl)propanoate (0.85 g, 3.87 mmol) at 0° C. simultaneously, and the mixture was stirred at room temperature for 16 hours. The reaction mixture was diluted with ice water (100 mL), extracted with ethyl acetate (2×50 mL) and washed with ice cold water (2×50 mL). The organic layer was dried over sodium sulfate and evaporated under reduced pressure to afford a residue that was was purified by silica gel column chromatography by eluting with 100% ethyl acetate to afford methyl (S)-2-((S)-2-((((S)-2-(3-chlorophenyl)-2,2-difluoro-1-phenylethoxy)carbonyl)amino)-3-cyclohexylpropanamido)-3-((S)-2-oxopyrrolidin-3-yl)propanoate (5). TLC system: 5% Methanol in dichloromethane Rf: 0.3; LCMS (ESI): m/z 634.56 [M+H]⁺

### (S)-2-(3-Chlorophenyl)-2,2-difluoro-1-phenylethyl ((S)-3-cyclohexyl-1-(((S)-1-hydroxy-3-((S)-2-oxopyrrolidin-3-yl)propan-2-yl)amino)-1-oxopropan-2-yl)carbamate (6)

To a stirred solution of methyl (S)-2-((S)-2-((((S)-2-(3-chlorophenyl)-2,2-difluoro-1-phenylethoxy)carbonyl)amino)-3-cyclohexylpropanamido)-3-((S)-2-oxopyrrolidin-3-yl)propanoate (5) (1.1 g, 1.7377 mmol) in dichloromethane (15 mL) was added 2M LiBH₄ in THE (0.86 mL, 1.7377 mmol) at 0° C. and the mixture was stirred for 3 hours. The progress of the reaction was monitored by TLC and LCMS. The reaction mixture was quenched with saturated NH₄Cl solution and extracted with dichloromethane (2×50 mL), dried over sodium sulfate, and concentrated under reduced pressure. The resulting compound was purified by normal phase chromatography and triturated with ether to afford (S)-2-(3-chlorophenyl)-2,2-difluoro-1-phenylethyl ((S)-3-cyclohexyl-1-(((S)-1-hydroxy-3-((S)-2-oxopyrrolidin-3-yl)propan-2-yl)amino)-1-oxopropan-2-yl)carbamate (6). TLC system: 5% Methanol in dichloromethane Rf: 0.4; LCMS (ESI): m/z=606652 [M+H]⁺

### (S)-2-(3-chlorophenyl)-2,2-difluoro-1-phenylethyl ((S)-3-cyclohexyl-1-oxo-1-(((S)-1-oxo-3-((S)-2-oxopyrrolidin-3-yl)propan-2-yl)amino)propan-2-yl)carbamate (A20-1)

To a stirred solution of (S)-2-(3-chlorophenyl)-2,2-difluoro-1-phenylethyl ((S)-3-cyclohexyl-1-(((S)-1-hydroxy-3-((S)-2-oxopyrrolidin-3-yl)propan-2-yl)amino)-1-oxopropan-2-yl)carbamate (6) (800 mg, 1.322 mmol) in ethyl acetate (20 mL) was added Dess-Martin periodinane (840 mg, 1.983 mmol) at 0° C. and the solution was stirred at room temperature for 16 hours. The progress of the reaction was monitored by TLC and LCMS. The reaction mass was filtered through a Celite® pad, which was washed with ethyl acetate (100 mL). Then the organic layer was washed with 10% sodium thiosulfate solution (2×100 mL) followed by saturated sodium bicarbonate solution (2×100 mL), water (1×100 mL), and brine (1×100 mL). The organic layer was dried over anhydrous Na₂SO₄, filtered and concentrated to provide a residue that was purified by normal phase chromatography to afford (S)-2-(3-chlorophenyl)-2,2-difluoro-1-phenylethyl ((S)-3-cyclohexyl-1-oxo-1-(((S)-1-oxo-3-((S)-2-oxopyrrolidin-3-yl)propan-2-yl)amino)propan-2-yl)carbamate (A20-1). TLC system: 10% Methanol in DCM R_{f}: 0.55; LCMS (ESI): m/z 604.3 [M+H]⁺

### A20 Diastereomer 2 (A20-2)

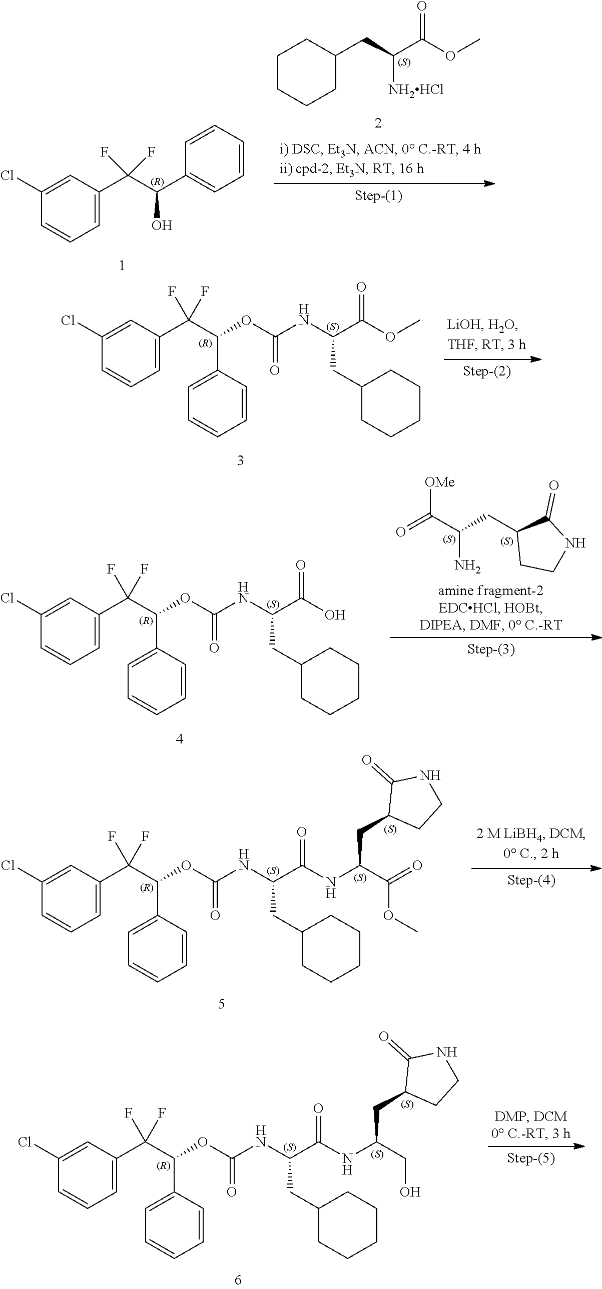

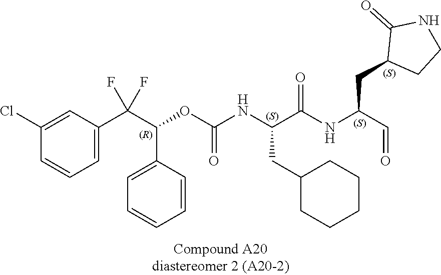

### Methyl (S)-2-((((R)-2-(3-chlorophenyl)-2,2-difluoro-1-phenylethoxy)carbonyl)amino)-3-cyclohexylpropanoate (3)

To a stirred solution of (R)-2-(3-chlorophenyl)-2,2-difluoro-1-phenylethan-1-ol (1) (3.2 g, 11.94 mmol) in ACN (35 mL) was added N, N′-disuccinamidyl carbonate (4.6 g, 17.910 mmol), followed by triethylamine (4 mL, 35.82 mmol) at 0° C., and the reaction mixture was stirred at room temperature for 4 hours. The progress of the reaction was monitored by TLC. The reaction mass was used directly in the subsequent reaction.

In another flask, methyl (S)-2-amino-3-cyclohexylpropanoate hydrochloride (2) (2.65 g, 14.328 mmol) was taken in ACN (35 mL), and treated with triethylamine (4 mL, 35.82 mmol) The resulting reaction mixture was stirred for 5 minutes, then added to the above prepared reaction mass drop-wise and the reaction mixture was stirred at room temperature for 16 hours. The reaction mixture was quenched with ice water (150 mL) and extracted with ethyl acetate (2×100 mL). The combined organic layers were washed with brine solution (100 mL), dried over sodium sulfate and evaporated under reduced pressure to afford a residue that was purified by reverse phase chromatography to afford methyl (S)-2-((((R)-2-(3-chlorophenyl)-2,2-difluoro-1-phenylethoxy)carbonyl)amino)-3-cyclohexylpropanoate (3). TLC system: 10% Ethyl acetate in hexane Rf: 0.55; LCMS (ESI): m/z 480.2 [M+H]⁺

### (S)-2-((((R)-2-(3-chlorophenyl)-2,2-difluoro-1-phenylethoxy)carbonyl)amino)-3-cyclohexylpropanoic acid (4)

To a stirred solution of methyl (S)-2-((((R)-2-(3-chlorophenyl)-2,2-difluoro-1-phenylethoxy)carbonyl)amino)-3-cyclohexylpropanoate (3) (2.5 g, 5.219 mmol) in THE (25 mL), water (12.5 mL) was added lithium hydroxide (0.394 g, 10.7438 mmol) at room temperature and stirred at room temperature for 3 hours. The progress of the reaction was monitored by TLC and LCMS. The reaction mixture was completely distilled under reduced pressure, and the compound was acidified with aq. 1N HCl solution up to pH˜2, extracted with dichloromethane (2×100 mL), dried over sodium sulfate, and concentrated under reduced pressure to afford (S)-2-((((R)-2-(3-chlorophenyl)-2,2-difluoro-1-phenylethoxy)carbonyl)amino)-3-cyclohexylpropanoic acid (4). TLC system: 50% Ethyl acetate in hexane Rf: 0.1; LCMS (ESI): m/z=466.14 [M+H]⁺

### Methyl (S)-2-((S)-2-((((R)-2-(3-chlorophenyl)-2,2-difluoro-1-phenylethoxy)carbonyl)amino)-3-cyclohexylpropanamido)-3-((S)-2-oxopyrrolidin-3-yl)propanoate (5)

To a stirred solution of (S)-2-((((R)-2-(3-chlorophenyl)-2,2-difluoro-1-phenylethoxy)carbonyl)amino)-3-cyclohexylpropanoic acid (4) (0.5 g, 1.075 mmol) in DMF (5 mL) was added EDC·HCl (0.3 g, 1.612 mmol), HOBt (0.21 g, 1.612 mmol), DIPEA (0.46 mL, 3.225 mmol) and methyl (S)-2-amino-3-((S)-2-oxopyrrolidin-3-yl)propanoate (0.23 g, 1.29 mmol) at 0° C. simultaneously, and the mixture was stirred at room temperature for 16 hours. The reaction mixture was diluted with ice water (100 mL), extracted with ethyl acetate (2×50 mL) and washed with ice cold water (2×50 mL). The organic layer was dried over sodium sulfate and evaporated under reduced pressure to afford a residue that was purified by silica gel column chromatography by eluting with 100% ethyl acetate to afford methyl (S)-2-((S)-2-((((R)-2-(3-chlorophenyl)-2,2-difluoro-1-phenylethoxy)carbonyl)amino)-3-cyclohexylpropanamido)-3-((S)-2-oxopyrrolidin-3-yl)propanoate (5). TLC system: 5% Methanol in dichloromethane Rf: 0.3; LCMS (ESI): m/z 634.3 [M+H]⁺

### (R)-2-(3-Chlorophenyl)-2,2-difluoro-1-phenylethyl ((S)-3-cyclohexyl-1-(((S)-1-hydroxy-3-((S)-2-oxopyrrolidin-3-yl)propan-2-yl)amino)-1-oxopropan-2-yl)carbamate (6)

To a stirred solution of methyl (S)-2-((S)-2-((((R)-2-(3-chlorophenyl)-2,2-difluoro-1-phenylethoxy)carbonyl)amino)-3-cyclohexylpropanamido)-3-((S)-2-oxopyrrolidin-3-yl)propanoate (5) (0.35 g, 0.552 mmol) in dichloromethane (5 mL) was added 2M LiBH₄ in THE (0.5 mL, 1.1058 mmol) at 0° C., and the mixture was stirred for 3 hours. The progress of the reaction was monitored by TLC and LCMS. The reaction mixture was quenched with saturated NH₄Cl solution and extracted with dichloromethane (2×50 mL), and dried over sodium sulfate. The mixture was concentrated under reduced pressure and the residue was purified by normal phase chromatography and triturated with ether to afford (R)-2-(3-chlorophenyl)-2,2-difluoro-1-phenylethyl ((S)-3-cyclohexyl-1-(((S)-1-hydroxy-3-((S)-2-oxopyrrolidin-3-yl)propan-2-yl)amino)-1-oxopropan-2-yl)carbamate (6). TLC system: 5% Methanol in dichloromethane Rf: 0.4; LCMS (ESI): m/z=606.3 [M+H]⁺

### (R)-2-(3-chlorophenyl)-2,2-difluoro-1-phenylethyl ((S)-3-cyclohexyl-1-oxo-1-(((S)-1-oxo-3-((S)-2-oxopyrrolidin-3-yl)propan-2-yl)amino)propan-2-yl)carbamate (A20-2)

To a stirred solution of (R)-2-(3-chlorophenyl)-2,2-difluoro-1-phenylethyl ((S)-3-cyclohexyl-1-(((S)-1-hydroxy-3-((S)-2-oxopyrrolidin-3-yl)propan-2-yl)amino)-1-oxopropan-2-yl)carbamate (6) (0.300 g, 0.49 mmol) in EtOAc (60 mL) was added Dess-Martin periodinane (0.63 g, 1.40 mmol) at 0° C. and the mixture was stirred at room temperature for 3 hours. The reaction mixture was diluted with EtOAc (20 mL) and washed with saturated hypo solution (3×20 mL) followed by saturated NaHCO₃ solution (3×20 mL). The organic layer was dried over anhydrous Na₂SO₄, filtered and concentrated to yield a residue that was purified by reverse phase column to afford (1-(3-chlorophenyl)cyclopropyl)(4-fluorophenyl)methyl ((S)-1-oxo-1-(((S)-1-oxo-3-((S)-2-oxopyrrolidin-3-yl)propan-2-yl)amino)hexan-2-yl)carbamate (A20-2). TLC system: 10% Methanol in dichloromethane Rf: 0.5; LCMS (ESI): m/z 603.23 (M+H)⁺

### Example 3: Synthesis of Compound A25 Diastereomers 1 and 2

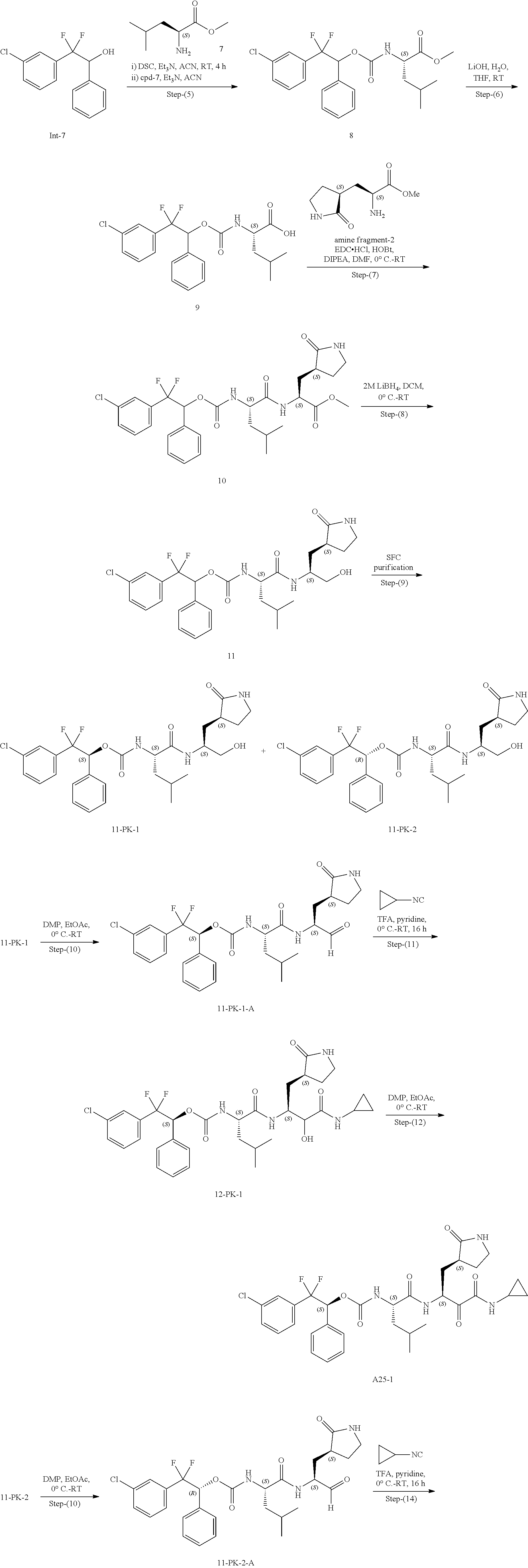

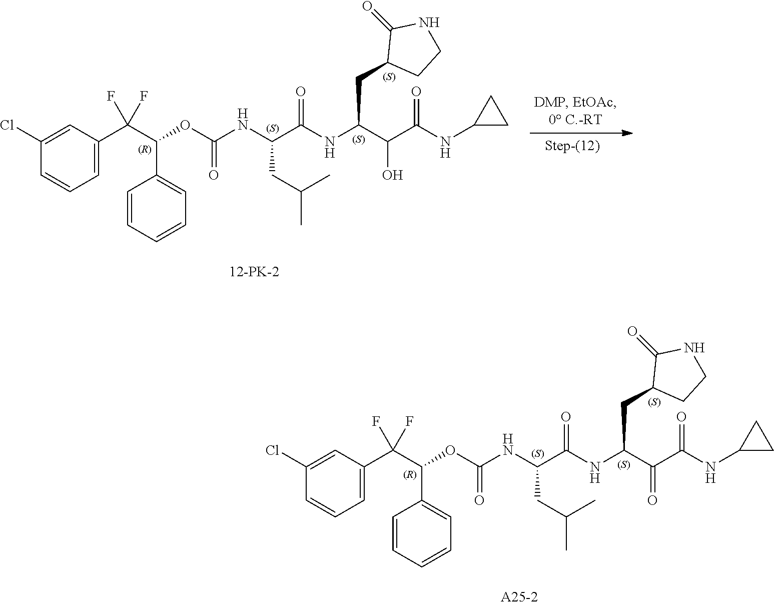

### Methyl ((2-(3-chlorophenyl)-2,2-difluoro-1-phenylethoxy)carbonyl)-L-leucinate (8)

To a stirred solution of 2-(3-chlorophenyl)-2,2-difluoro-1-phenylethan-1-ol (Int-7) (3.8 g, 14.179 mmol) in ACN (40 mL) was added N,N′disuccinamidyl carbonate (5.4 g, 21.268 mmol), followed by triethylamine (5.9 mL, 42.537 mmol) at 0° C., and the mixture was stirred at room temperature for 4 hours. The progress of the reaction was monitored by TLC. The reaction mass was used directly in the subsequent reaction.

In another flask, methyl L-leucinate hydrochloride (7) (3.89 g, 21.268 mmol) was taken in ACN (40 mL), and treated with triethylamine (5.9 mL, 42.537 mmol). The resulting reaction mixture was stirred for 5 minutes, then the above prepared reaction mass was added drop-wise and the reaction mixture stirred at room temperature for 16 hours. The reaction mixture was quenched with ice water (150 mL) and extracted with ethyl acetate (2×100 mL). The combined organic layers were washed with brine solution (150 mL), dried over sodium sulfate and evaporated under reduced pressure, and the residue was purified by normal phase chromatography by using petroleum ether and ethyl acetate as mobile phases to afford methyl ((2-(3-chlorophenyl)-2,2-difluoro-1-phenylethoxy)carbonyl)-L-leucinate (8). TLC system: 20% Ethyl acetate/Pet ether R_{f}: 0.4; LCMS (ESI): m/z 440.39 [M+H]⁺

### ((2-(3-Chlorophenyl)-2,2-difluoro-1-phenylethoxy)carbonyl)-L-leucine (9)

To a stirred solution of methyl ((2-(3-chlorophenyl)-2,2-difluoro-1-phenylethoxy)carbonyl)-L-leucinate (8) (2.5 g, 5.694 mmol) in THE (25 mL), water (25 mL) was added lithium hydroxide (717 mg, 17.084 mmol) at room temperature, and the mixture was stirred at room temperature for 2 hours. The progress of the reaction was monitored by TLC and LCMS. Excess THE was removed under reduced pressure, and the resulting residue was acidified with aq. 1N HCl solution up to pH˜2. The mixture was extracted with DCM (2×100 mL), and the combined organic layers were washed with water (100 mL) brine solution (150 mL), dried over sodium sulfate, and concentrated under reduced pressure to afford ((2-(3-chlorophenyl)-2,2-difluoro-1-phenylethoxy)carbonyl)-L-leucine (9). TLC system: 10% Methanol in dichloromethane Rf: 0.1; LCMS (ESI): m/z 448.30 [M+Na]⁺

### Methyl (2S)-2-((2S)-2-(((2-(3-chlorophenyl)-2,2-difluoro-1-phenylethoxy)carbonyl)amino)-4-methylpentanamido)-3-((S)-2-oxopyrrolidin-3-yl)propanoate (10)

To a stirred solution of ((2-(3-chlorophenyl)-2,2-difluoro-1-phenylethoxy)carbonyl)-L-leucine (9) (2 g, 4.70 mmol) in DMF (20 mL) was added EDC-HCl (1.34 g, 7.058 mmol), HOBt (0.952 g, 7.058 mmol), DIPEA (2.5 mL, 14.61176 mmol) and methyl (S)-2-amino-3-((S)-2-oxopyrrolidin-3-yl)propanoate hydrochloride (1.567 g, 7.058 mmol) at 0° C. simultaneously, and the mixture was stirred at room temperature for 16 hours. The reaction mixture was diluted with ice water (100 mL) and extracted with ethyl acetate (2×100 mL), then the organic layer was dried over sodium sulfate and evaporated under reduced pressure to afford a residue that was purified by normal phase chromatography to afford methyl (2S)-2-((2S)-2-(((2-(3-chlorophenyl)-2,2-difluoro-1-phenylethoxy)carbonyl)amino)-4-methylpentanamido)-3-((S)-2-oxopyrrolidin-3-yl)propanoate (10). TLC system: 10% MeOH/DCM Rf: 0.45; LCMS (ESI): m/z=594.62 [M+H]⁺

### 2-(3-Chlorophenyl)-2,2-difluoro-1-phenylethyl ((S)-1-(((S)-1-hydroxy-3-((S)-2-oxopyrrolidin-3-yl)propan-2-yl)amino)-4-methyl-1-oxopentan-2-yl)carbamate (11)

To a stirred solution of methyl (2S)-2-((2S)-2-(((2-(3-chlorophenyl)-2,2-difluoro-1-phenylethoxy)carbonyl)amino)-4-methylpentanamido)-3-((S)-2-oxopyrrolidin-3-yl)propanoate (10) (1 g, 1.686 mmol,) in dichloromethane (10 mL) was added 2M LiBH₄ in THE (1.68 mL, 3.372 mmol) at 0° C., and the mixture was stirred for 2 hours. The progress of the reaction was monitored by TLC and LCMS. The reaction mixture was quenched with saturated NH₄Cl solution and extracted with dichloromethane (2×50 mL), then dried over sodium sulfate and concentrated under reduced pressure to afford 2-(3-chlorophenyl)-2,2-difluoro-1-phenylethyl((S)-1-(((S)-1-hydroxy-3-((S)-2-oxopyrrolidin-3-yl)propan-2-yl)amino)-4-methyl-1-oxopentan-2-yl)carbamate (11). TLC system: 10% Methanol in dichloromethane Rf: 0.35; LCMS (ESI): m/z 566.49 [M+H]⁺

### (S)-2-(3-chlorophenyl)-2,2-difluoro-1-phenylethyl ((S)-1-(((S)-1-hydroxy-3-((S)-2-oxopyrrolidin-3-yl)propan-2-yl)amino)-4-methyl-1-oxopentan-2-yl)carbamate (11-PK-1) and (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-4-methyl-1-oxopentan-2-yl)carbamate (11-PK-2)

2-(3-chlorophenyl)-2,2-difluoro-1-phenylethyl ((S)-1-(((S)-1-hydroxy-3-((S)-2-oxopyrrolidin-3-yl)propan-2-yl)amino)-4-methyl-1-oxopentan-2-yl)carbamate (1) (7 g, 12.38 mmol) was purified by SFC purification to afford (S)-2-(3-chlorophenyl)-2,2-difluoro-1-phenylethyl ((S)-1-(((S)-1-hydroxy-3-((S)-2-oxopyrrolidin-3-yl)propan-2-yl)amino)-4-methyl-1-oxopentan-2-yl)carbamate (11-PK-1) and (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-4-methyl-1-oxopentan-2-yl)carbamate (11-PK-2). LCMS (ESI): m/z 566.52 [M+H]⁺

### (S)-2-(3-Chlorophenyl)-2,2-difluoro-1-phenylethyl ((S)-4-methyl-1-oxo-1-(((S)-1-oxo-3-((S)-2-oxopyrrolidin-3-yl)propan-2-yl)amino)pentan-2-yl)carbamate (11-PK-1-A)

To a stirred solution of (S)-2-(3-chlorophenyl)-2,2-difluoro-1-phenylethyl ((S)-1-(((S)-1-hydroxy-3-((S)-2-oxopyrrolidin-3-yl)propan-2-yl)amino)-4-methyl-1-oxopentan-2-yl)carbamate (11-PK-1) (1.8 g, 3.185 mmol) in ethyl acetate (15 mL) was added Dess-Martin periodinane (4.5 g, 10.83 mmol) at 0° C., and the mixture was stirred at room temperature for 16 hours. The progress of the reaction was monitored by TLC and LCMS. The reaction mixture was diluted with ethyl acetate (20 mL) and washed with saturated NaHCO₃ solution (3×20 mL) followed by saturated hypo solution (3×20 mL). The organic layer was dried over anhydrous Na₂SO₄, filtered and concentrated to provide a residue that was purified by normal phase purification to afford (S)-2-(3-chlorophenyl)-2,2-difluoro-1-phenylethyl ((S)-4-methyl-1-oxo-1-(((S)-1-oxo-3-((S)-2-oxopyrrolidin-3-yl)propan-2-yl)amino)pentan-2-yl)carbamate (11-PK-1-A). TLC system: 10% Methanol in DCM R_{f}: 0.55; LCMS (ESI): m/z 564.2 [M+H]⁺

### (S)-2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (12-PK-1)

To a stirred solution of (S)-2-(3-chlorophenyl)-2,2-difluoro-1-phenylethyl ((S)-4-methyl-1-oxo-1-(((S)-1-oxo-3-((S)-2-oxopyrrolidin-3-yl)propan-2-yl)amino)pentan-2-yl)carbamate (11-PK-1-A) (800 mg, 1.42 mmol) in DCM (6 mL), to which was added pyridine (0.7 mL, 7.1 mmol), isocyanocyclopropane (114 mg, 1.7 mmol), and TFA (80 mg, 0.71 mmol) at 0° C., and the mixture was stirred at room temperature for 16 hours. The progress of the reaction was monitored by TLC and LCMS. The reaction mixture was diluted with dichloromethane and washed with 1N HCl (2×30 mL) followed by water (20 mL) and brine (20 mL). The organic layer was dried over anhydrous Na₂SO₄ and evaporated under reduced pressure to afford (S)-2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (12-PK-1). TLC system: 10% MeOH in DCM Rf: 0.25; LCMS (ESI): m/z 649.62 [M+H]⁺

### (S)-2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound A25-1)

To a stirred solution (S)-2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (12-PK-1) (900 mg, 1.388 mmol) in ethyl acetate (8 mL) was added Dess-Martin periodinane (1.4 g, 3.47 mmol) at 0° C., and the mixture was stirred at room temperature for 16 hours. The progress of the reaction was monitored by TLC and LCMS. The reaction mixture was filtered through a Celite® pad and washed with ethyl acetate (25 mL), and the filtrate was washed with hypo solution (3×40 mL) followed by saturated NaHCO₃ solution (3×20 mL). The organic layer was dried over anhydrous Na₂SO₄, filtered, and concentrated to afford a residue that was purified by prep HPLC to afford (S)-2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound A25-1). TLC system: 10% MeOH in DCM Rf: 0.3; LCMS (ESI): m/z 647.3 [M+H]⁺

### (R)-2-(3-chlorophenyl)-2,2-difluoro-1-phenylethyl ((S)-4-methyl-1-oxo-1-(((S)-1-oxo-3-((S)-2-oxopyrrolidin-3-yl)propan-2-yl)amino)pentan-2-yl)carbamate (11-PK-2-A)

To a stirred solution of (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-4-methyl-1-oxopentan-2-yl)carbamate (11-PK-2) (1.9 g, 3.36 mmol) in ethyl acetate (15 mL) was added Dess-Martin periodinane (5.25 g, 11.76 mmol) at 0° C., and the mixture was stirred at room temperature for 16 hours. The progress of the reaction was monitored by TLC and LCMS. The reaction mixture was diluted with ethyl acetate (20 mL) and washed with saturated NaHCO₃ solution (3×20 mL) followed by saturated hypo solution (3×20 mL). The organic layer was dried over anhydrous Na₂SO₄, filtered, and concentrated to afford a residue that was purified by normal phase purification to afford (R)-2-(3-chlorophenyl)-2,2-difluoro-1-phenylethyl ((S)-4-methyl-1-oxo-1-(((S)-1-oxo-3-((S)-2-oxopyrrolidin-3-yl)propan-2-yl)amino)pentan-2-yl)carbamate (11-PK-2-A). TLC system: 10% Methanol in DCM R_{f}: 0.55; LCMS (ESI): m/z 564.3 [M+H]⁺

### (R)-2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (12-PK-2)

To a stirred solution of (R)-2-(3-chlorophenyl)-2,2-difluoro-1-phenylethyl ((S)-4-methyl-1-oxo-1-(((S)-1-oxo-3-((S)-2-oxopyrrolidin-3-yl)propan-2-yl)amino)pentan-2-yl)carbamate (11-PK-2-A) (1.1 g, 1.95 mmol) in DCM (8 mL) was added pyridine (0.8 mL, 9.75 mmol), isocyanocyclopropane (214 mg, 3.19 mmol), and TFA (111 mg, 0.95 mmol) at 0° C., and the mixture was stirred at room temperature for 16 hours. The progress of the reaction was monitored by TLC and LCMS. The reaction mixture was diluted with dichloromethane and washed with 1N HCl (2×30 mL) followed by water (20 mL) and brine (20 mL). The organic layer was dried over anhydrous Na₂SO₄ and evaporated under reduced pressure to afford (R)-2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (12-PK-2). TLC system: 10% MeOH in DCM Rf: 0.25; LCMS (ESI): m/z 649.62 [M+H]⁺

### (R)-2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound A25-2)

To a stirred solution (R)-2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (12-PK-2) (1.1 g (crude), 1.69 mmol) in ethyl acetate (12 mL) was added Dess-Martin periodinane (2.4 g, 5.94 mmol) at 0° C., and the mixture was stirred at room temperature for 16 hours. The progress of the reaction was monitored by TLC and LCMS. Reaction mixture was filtered through a Celite® pad and washed with ethyl acetate (25 mL), and then the filtrate was washed with hypo solution (3×40 mL) followed by saturated NaHCO₃ solution (3×20 mL). The organic layer was dried over anhydrous Na₂SO₄, filtered and concentrated to provide a residue that was purified by prep HPLC to afford (R)-2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound A25-2). TLC system: 10% MeOH in DCM Rf: 0.3; LCMS (ESI): m/z 647.3 [M+H]⁺

### Example 4: Synthesis of Compound A26P and A26

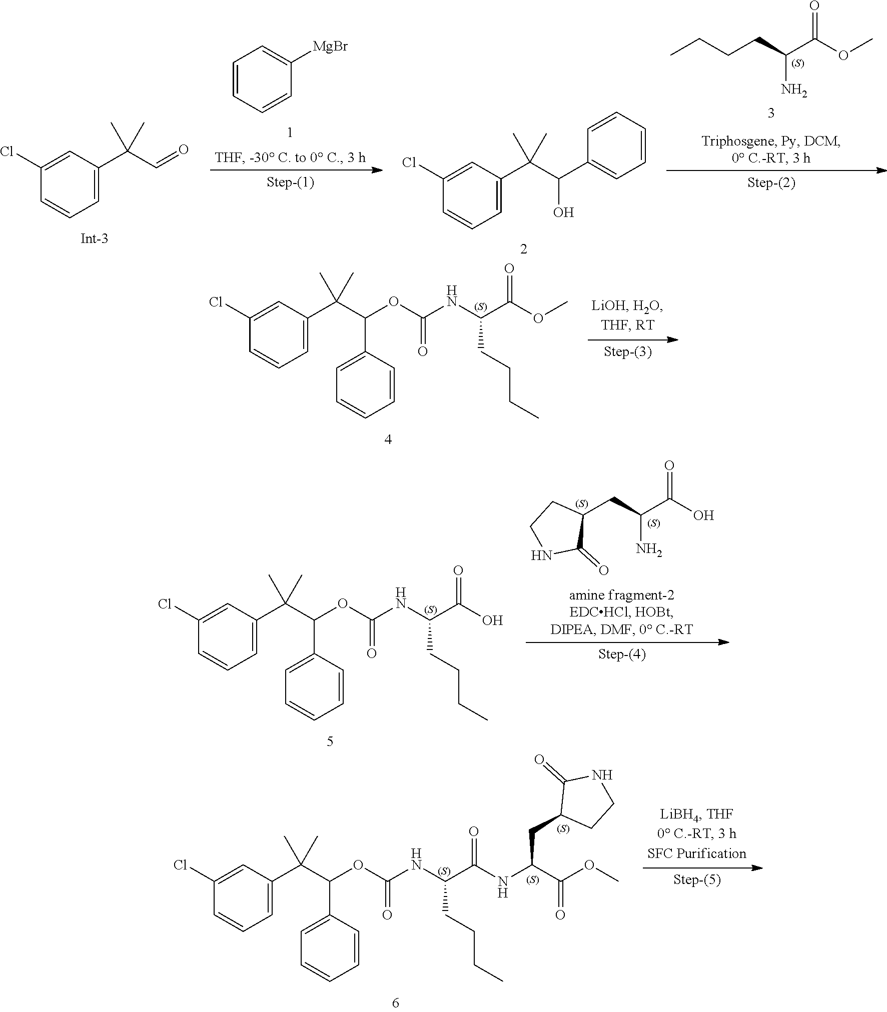

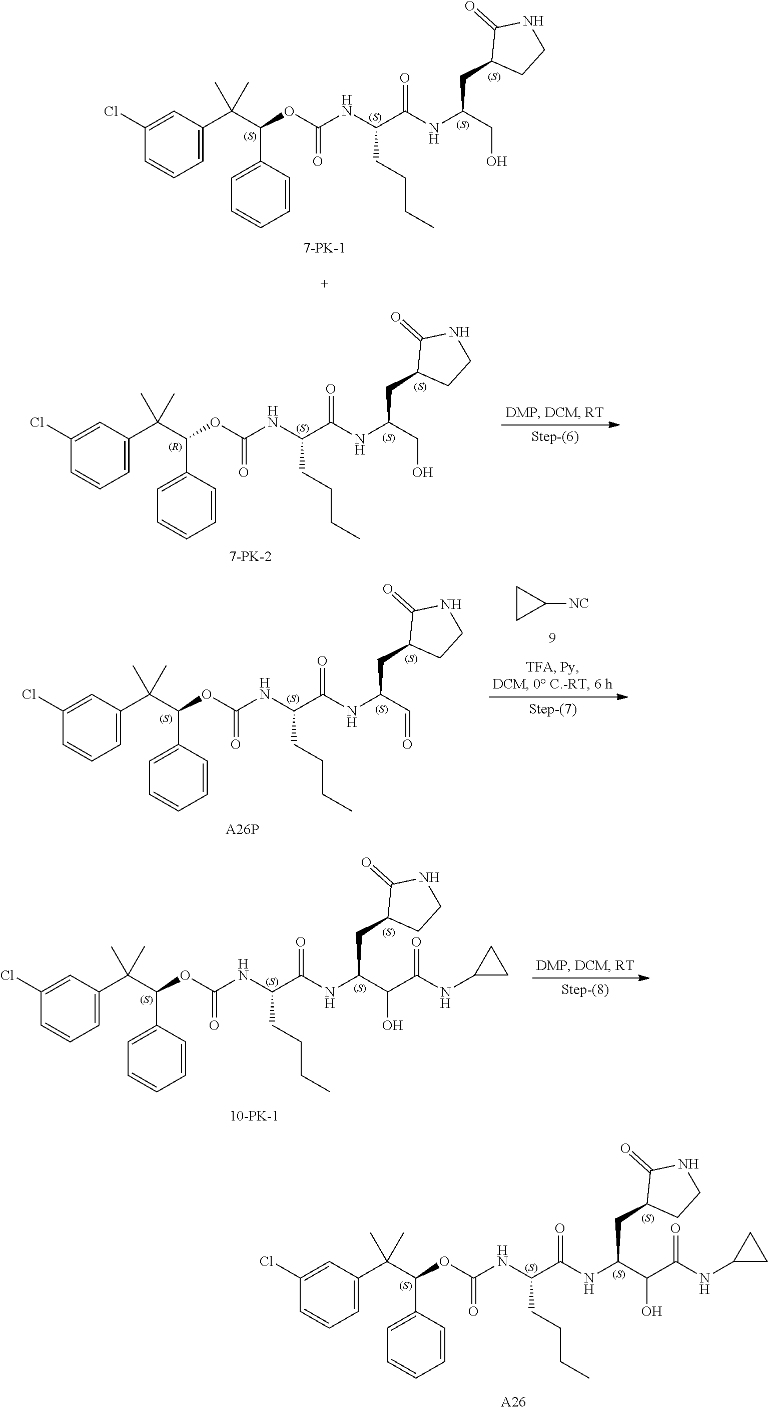

### 2-(3-Chlorophenyl)-2-methyl-1-phenylpropan-1-ol (2)

To a stirred solution of 2-(3-chlorophenyl)-2-methylpropanal (Int-3) (10 g, 54.94 mmol) in THE (100 mL) was added 4-chloro phenyl magnesium bromide (1) (109.89 mL, 109.89 mmol) at −30° C. and the reaction mixture was stirred at 0° C. for 3 hours. The progress of the reaction was monitored by TLC. The reaction mixture was quenched with saturated NH₄Cl (150 mL) and filtered through a Celite® bed and washed with ethyl acetate (2×150 mL), dried over sodium sulfate and evaporated under reduced pressure. The residue was purified by silica gel column chromatography by eluting with 5% ethyl acetate in hexane to afford 2-(3-chlorophenyl)-2-methyl-1-phenylpropan-1-ol (2). TLC system: 10% Ethyl acetate in hexane Rf: 0.3; LCMS (ESI): m/z=243.18 [M−OH]⁺

### Methyl (2S)-2-(((2-(3-chlorophenyl)-2-methyl-1-phenylpropoxy)carbonyl)amino)hexanoate (4)

To a stirred solution of 2-(3-chlorophenyl)-2-methyl-1-phenylpropan-1-ol (2) (10 g, 38.46 mmol) (2) in DCM (100 mL) was added pyridine (7.6 mL, 96.15 mmol). Next was added methyl (S)-2-aminohexanoate (3) (16.25 g, 57.69 mmol) and triphosgene (5.6 g, 19.23 mmol) at 0° C., and the reaction mixture was stirred at room temperature for 3 hours. The progress of the reaction was monitored by TLC and LCMS. The reaction mixture was acidified with 1N HCl (25 mL) and extracted with ethyl acetate (2×25 mL), then the organic layer was dried over sodium sulfate, filtered and evaporated under reduced pressure. The residue was purified by combi-flash, and eluted at 10% ethyl acetate in petroleum ether to afford methyl (2S)-2-(((2-(3-chlorophenyl)-2-methyl-1-phenylpropoxy) carbonyl)amino)hexanoate (4). TLC system: 10% Ethyl acetate in hexane Rf: 0.3; LCMS (ESI): m/z=431.12 [M+H]⁺

### (2S)-2-(((2-(3-chlorophenyl)-2-methyl-1-phenylpropoxy)carbonyl)amino)hexanoic acid (5)

To a stirred solution of methyl (2S)-2-(((2-(3-chlorophenyl)-2-methyl-1-phenylpropoxy)carbonyl)amino)hexanoate (4) (12 g, 27.84 mmol) in THE (80 mL), water (30 mL) was added lithium hydroxide (2.28 g, 55.68 mmol) at room temperature, and the reaction mixture was stirred at room temperature for 3 hours. The progress of the reaction was monitored by TLC and LCMS. The reaction mixture was completely evaporated under reduced pressure, and the residue acidified with aqueous 1N HCl solution up to pH˜2. The solution was extracted with ethyl acetate (2×60 mL), dried over sodium sulfate, and concentrated under reduced pressure to afford (2S)-2-(((2-(3-chlorophenyl)-2-methyl-1-phenylpropoxy)carbonyl)amino)hexanoic acid (5). TLC system: 5% Methanol in dichloromethane Rf: 0.1; LCMS (ESI): m/z 440.38 [M+Na]⁺

### Methyl (2S)-2-((2S)-2-(((2-(3-chlorophenyl)-2-methyl-1-phenylpropoxy)carbonyl)amino) hexanamido)-3-((S)-2-oxopyrrolidin-3-yl)propanoate (6)

To a stirred solution of (2S)-2-(((2-(3-chlorophenyl)-2-methyl-1-phenylpropoxy)carbonyl)amino)hexanoic acid (5) (8 g, 25.23 mmol) in DMF (100 mL) was added EDC·HCl (7.23 g, 37.85 mmol), HOBt (5.1 g, 37.85 mmol), DIPEA (19.79 mL, 113.55 mmol) and methyl (S)-2-amino-3-((S)-2-oxopyrrolidin-3-yl)propanoate (8.36 g, 37.85 mmol) at 0° C. simultaneously, and the mixture was stirred at room temperature for 16 hours. The reaction mixture was diluted with ice water (100 mL), extracted with ethyl acetate (2×50 mL) and washed with ice cold water (2×50 mL). The organic layer was dried over sodium sulfate and evaporated under reduced pressure to afford a residue that was purified by silica gel column chromatography by eluting with 100% ethyl acetate to afford methyl (2S)-2-((2S)-2-(((2-(3-chlorophenyl)-2-methyl-1-phenylpropoxy)carbonyl)amino)hexanamido)-3-((S)-2-oxopyrrolidin-3-yl)propanoate (6). TLC system: 5% Methanol in dichloromethane Rf: 0.4; LCMS (ESI): m/z=586.55 [M+H]⁺

### (S)-2-(3-Chlorophenyl)-2-methyl-1-phenylpropyl ((S)-1-(((S)-1-hydroxy-3-((S)-2-oxopyrrolidin-3-yl)propan-2-yl)amino)-1-oxohexan-2-yl)carbamate (7-PK-1) and (R)-2-(3-chlorophenyl)-2-methyl-1-phenylpropyl ((S)-1-(((S)-1-hydroxy-3-((S)-2-oxopyrrolidin-3-yl)propan-2-yl)amino)-1-oxohexan-2-yl)carbamate (7-PK-2)

To a stirred solution of methyl (2S)-2-((2S)-2-(((2-(3-chlorophenyl)-2-methyl-1-phenylpropoxy)carbonyl)amino)hexanamido)-3-((S)-2-oxopyrrolidin-3-yl)propanoate (6) (8 g, 13.67 mmol) in THE (100 mL) was added 2M LiBH₄ in THE (13.67 mL, 27.35 mmol) at 0° C. and the mixture was stirred for 3 hours. The progress of the reaction was monitored by TLC and LCMS. The reaction mixture was quenched with saturated NH₄Cl solution and extracted with EtOAc (2×50 mL), dried over sodium sulfate, and concentrated under reduced pressure to afford a residue that was purified by silica gel column chromatography by eluting with 100% ethyl acetate to afford 2-(3-chlorophenyl)-2-methyl-1-phenylpropyl ((S)-1-(((S)-1-hydroxy-3-((S)-2-oxopyrrolidin-3-yl)propan-2-yl)amino)-1-oxohexan-2-yl)carbamate (7).

Compound (7) was purified by SFC to afforded (S)-2-(3-chlorophenyl)-2-methyl-1-phenylpropyl ((S)-1-(((S)-1-hydroxy-3-((S)-2-oxopyrrolidin-3-yl)propan-2-yl)amino)-1-oxohexan-2-yl)carbamate (7-PK-1) and (R)-(4-chlorophenyl)(1-(3-chlorophenyl)cyclopropyl)methyl (R)-2-(3-chlorophenyl)-2-methyl-1-phenylpropyl ((S)-1-(((S)-1-hydroxy-3-((S)-2-oxopyrrolidin-3-yl)propan-2-yl)amino)-1-oxohexan-2-yl)carbamate (7-PK-2). TLC system: 5% Methanol in dichloromethane Rf: 0.3; LCMS (ESI): m/z 558.57 [M+H]⁺

### (S)-2-(3-Chlorophenyl)-2-methyl-1-phenylpropyl ((S)-1-oxo-1-(((S)-1-oxo-3-((S)-2-oxopyrrolidin-3-yl)propan-2-yl)amino)hexan-2-yl)carbamate (A26 precursor)

To a stirred solution of (4-chlorophenyl)(1-(3-chlorophenyl)cyclopropyl)methyl ((S)-1-(((S)-1-hydroxy-3-((S)-2-oxopyrrolidin-3-yl)propan-2-yl)amino)-4-methyl-1-oxopentan-2-yl)carbamate (7-PK-1) (1 g, 1.79 mmol) in ethyl acetate (10 mL) was added Dess-Martin periodinane (1.14 g, 2.69 mmol) at 0° C., and the mixture was stirred at room temperature for 3 hours. The reaction mixture was diluted with ethyl acetate (20 mL) and washed with saturated hypo solution (3×20 mL), saturated NaHCO₃ solution (3×20 mL) and brine (1×20 mL). The organic layer was dried over anhydrous Na₂SO₄, filtered and concentrated to afford a residue that was purified by prep HPLC afford (S)-2-(3-chlorophenyl)-2-methyl-1-phenylpropyl ((S)-1-oxo-1-(((S)-1-oxo-3-((S)-2-oxopyrrolidin-3-yl)propan-2-yl)amino)hexan-2-yl)carbamate (A26 precursor). TLC system: 5% Methanol in dichloromethane Rf: 0.3; LCMS (ESI): m/z 556.3 [M+H]⁺

### (S)-2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (10-PK-1)

To a stirred solution of (S)-2-(3-chlorophenyl)-2-methyl-1-phenylpropyl ((S)-1-oxo-1-(((S)-1-oxo-3-((S)-2-oxopyrrolidin-3-yl)propan-2-yl)amino)hexan-2-yl)carbamate (A26 precursor) (450 mg, 0.81 mmol) was added pyridine (0.5 mL, 5 vol), isocyanocyclopropane (9) (86.83 mg, 1.29 mmol) and TFA (184 mg, 1.62 mmol) at 0° C., and the mixture was stirred at room temperature for 16 hours. The progress of the reaction was monitored by TLC and LCMS. The reaction mixture was diluted with dichloromethane and washed with 1N HCl (2×20 mL) followed by brine (1×20 mL). The organic layer was dried over anhydrous Na₂SO₄ and evaporated under reduced pressure to afford crude (S)-2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (10-PK-1). TLC system: 10% Methanol in dichloromethane Rf: 0.5; LCMS (ESI): m/z 641.66 [M+H]⁺

### (S)-2-(3-Chlorophenyl)-2-methyl-1-phenylpropyl ((S)-1-(((S)-4-(cyclopropylamino)-3,4-dioxo-1-((S)-2-oxopyrrolidin-3-yl)butan-2-yl)amino)-1-oxohexan-2-yl)carbamate (Compound A26)

To a stirred solution of (S)-2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (10 PK-1) (450 mg, 0.70 mmol) in ethyl acetate (10 mL) was added Dess-Martin periodinane (744 mg, 1.75 mmol) at 0° C., and the mixture was stirred at room temperature for 3 hours. The progress of the reaction was monitored by TLC and LCMS. The reaction mixture was filtered through a Celite® pad and washed with ethyl acetate (20 mL) and the filtrate was washed with hypo solution (3×20 mL) followed by saturated NaHCO₃ solution (3×20 mL). The organic layer was dried over anhydrous Na₂SO₄, filtered and concentrated to afford a residue that was purified by prep HPLC to afford (S)-2-(3-chlorophenyl)-2-methyl-1-phenylpropyl ((S)-1-(((S)-4-(cyclopropylamino)-3,4-dioxo-1-((S)-2-oxopyrrolidin-3-yl)butan-2-yl)amino)-1-oxohexan-2-yl)carbamate (Compound A26). TLC system: 10% Methanol in dichloromethane Rf: 0.4; LCMS (ESI): m/z 639.3 [M+H]⁺

### Example 5: Synthesis of Compound A27

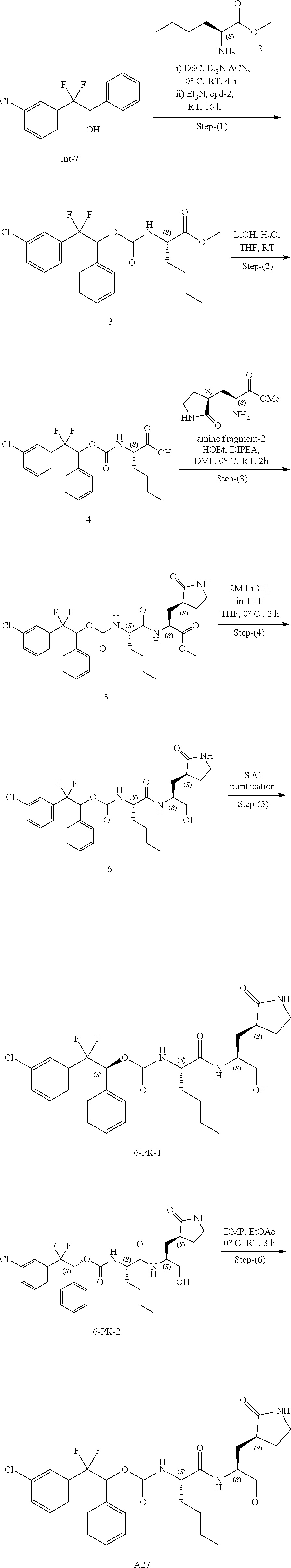

### Methyl (2S)-2-(((2-(3-chlorophenyl)-2,2-difluoro-1 phenylethoxy)carbonyl)amino) hexanoate (3)

To a stirred solution of 2-(3-chlorophenyl)-2,2-difluoro-1-phenylethan-1-ol (Int-7) (10 g, 37.313 mmol) in ACN (100 mL) was added N,N′disuccinamidyl carbonate (23.9 g, 93.283 mmol), followed by triethylamine (15.8 mL, 111.939 mmol) at 0° C., and the mixture was stirred at room temperature for 4 hours. The progress of the reaction was monitored by TLC. The reaction mass was used directly in the subsequent reaction.

In another flask, methyl (S)-2-aminohexanoate HCl (2) (16.9 g, 93.283 mmol) was taken in ACN (60 mL), and treated with triethylamine (15.8 mL, 111.939 mmol). The resulting reaction mixture was stirred for 5 min, then added to the above prepared reaction mass drop-wise and the reaction mixture stirred at room temperature for 16 hours. The reaction mixture was quenched with ice water (200 mL) and extracted with ethyl acetate (2×200 mL), then the combined organic layers were washed with brine solution (100 mL), dried over sodium sulfate and evaporated under reduced pressure to afford a residue that was purified by silica gel column chromatography to afford methyl (2S)-2-(((2-(3-chlorophenyl)-2,2-difluoro-1 phenylethoxy)carbonyl)amino)hexanoate (3). TLC system: 20% Ethyl acetate in hexane Rf: 0.3; LCMS (ESI): m/z 440.2 [M+H]⁺

### (2S)-2-(((2-(3-chlorophenyl)-2,2-difluoro-1-phenylethoxy)carbonyl)amino)hexanoic acid (4)

To a stirred solution of methyl (2S)-2-(((2-(3-chlorophenyl)-2,2-difluoro-1 phenylethoxy)carbonyl)amino)hexanoate (3) (10 g, 22.727 mmol) THE (80 mL), water (20 mL) was added lithium hydroxide (1.9 g, 45.454 mmol) at room temperature, and the mixture was stirred at room temperature for 3 hours. The progress of the reaction was monitored by TLC and LCMS. The reaction mixture was completely evaporated under reduced pressure, and then the residue was acidified with aq. 1N HCl solution up to pH˜2, extracted with ethyl acetate (2×200 mL), dried over sodium sulfate, and concentrated under reduced pressure to afford (2S)-2-(((2-(3-chlorophenyl)-2,2-difluoro-1-phenylethoxy)carbonyl)amino)hexanoic acid (4). TLC system: 60% Ethyl acetate in hexane Rf: 0.1; LCMS (ESI): m/z=448.2 [M+Na]⁺

### Methyl (2S)-2-((2S)-2-(((2-(3-chlorophenyl)-2,2-difluoro-1-phenylethoxy)carbonyl)amino)hexanamido)-3-((S)-2-oxopyrrolidin-3-yl)propanoate (5)

To a stirred solution of (2S)-2-(((2-(3-chlorophenyl)-2,2-difluoro-1-phenylethoxy)carbonyl)amino)hexanoic acid (4) (9 g, 21.176 mmol) in DMF (30 mL) was added EDC-HCl (6.06 g, 31.764 mmol), HOBt (4.3 g, 31.764 mmol), DIPEA (11.3 mL, 65.148 mmol) and methyl (S)-2-amino-3-((S)-2-oxopyrrolidin-3-yl)propanoate (7 g, 31.764 mmol) at 0° C. simultaneously, and the mixture was stirred at room temperature for 16 hours. The reaction mixture was diluted with ice water (200 mL), extracted with ethyl acetate (2×200 mL) and washed with ice cold water (2×100 mL). The organic layer was dried over sodium sulfate and evaporated under reduced pressure to afford a residue that was purified by silica gel column chromatography by eluting with 100% ethyl acetate to afford methyl (2S)-2-((2S)-2-(((2-(3-chlorophenyl)-2,2-difluoro-1-phenylethoxy)carbonyl)amino)hexanamido)-3-((S)-2-oxopyrrolidin-3-yl)propanoate (5). TLC system: 80% Ethyl acetate in hexane Rf: 0.3; LCMS (ESI): m/z 594.3 [M+H]⁺

### 2-(3-Chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6)

To a stirred solution of methyl (2S)-2-((2S)-2-(((2-(3-chlorophenyl)-2,2-difluoro-1-phenylethoxy)carbonyl)amino)hexanamido)-3-((S)-2-oxopyrrolidin-3-yl)propanoate (5) (8 g, 13.468 mmol) in THE (80 mL) was added 2M LiBH₄ in THE (14 mL, 26.93 mmol) at 0° C. and stirred for 3 hours. The progress of the reaction was monitored by TLC and LCMS. The reaction mixture was quenched with saturated NH₄Cl solution and extracted with ethyl acetate (2×200 mL), dried over sodium sulfate, and concentrated under reduced pressure to afford a residue that was purified by silica gel column chromatography to afford 2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6). TLC system: 5% Methanol in dichloromethane Rf: 0.4; LCMS (ESI): m/z=566.3 [M+H]⁺

### (S)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-1) & (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-2)

Compound (6) was purified by SFC to afford (S)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-1) and (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-2). TLC system: 5% Methanol in dichloromethane Rf: 0.3; LCMS (ESI): m/z 566.3 [M+H]⁺

### 2-(3-Chlorophenyl)-2,2-difluoro-1-phenylethyl ((S)-1-oxo-1-(((S)-1-oxo-3-((S)-2-oxopyrrolidin-3-yl)propan-2-yl)amino)hexan-2-yl)carbamate (Compound A27)

To a stirred solution of 2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6) (300 mg, 0.50 mmol) was dissolved in ethyl acetate (3 mL) was added Dess-Martin periodinane (674 mg, 1.59 mmol) at 0° C., and the mixture was stirred at room temperature for 3 hours. The reaction mixture was diluted with ethyl acetate (20 mL) and washed with saturated hypo solution (3×20 mL), saturated NaHCO₃ solution (3×20 mL), and brine (1×20 mL). The organic layer was dried over anhydrous Na₂SO₄, filtered, and concentrated to afford a residue that was purified by Prep HPLC afford 2-(3-chlorophenyl)-2,2-difluoro-1-phenylethyl ((S)-1-oxo-1-(((S)-1-oxo-3-((S)-2-oxopyrrolidin-3-yl)propan-2-yl)amino)hexan-2-yl)carbamate (Compound A27). TLC system: 5% Methanol in dichloromethane Rf: 0.3; LCMS (ESI): m/z 564.3 [M+H]⁺

### Example 6: Synthesis of Compound A28 Diastereomers 1 and 2

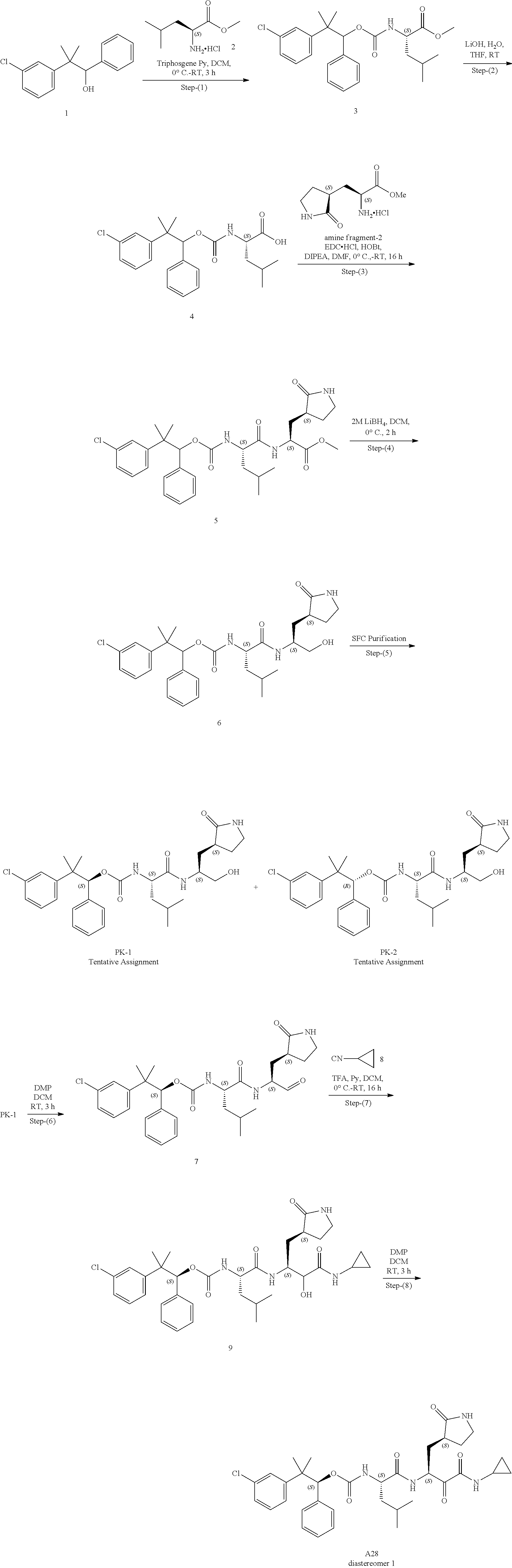

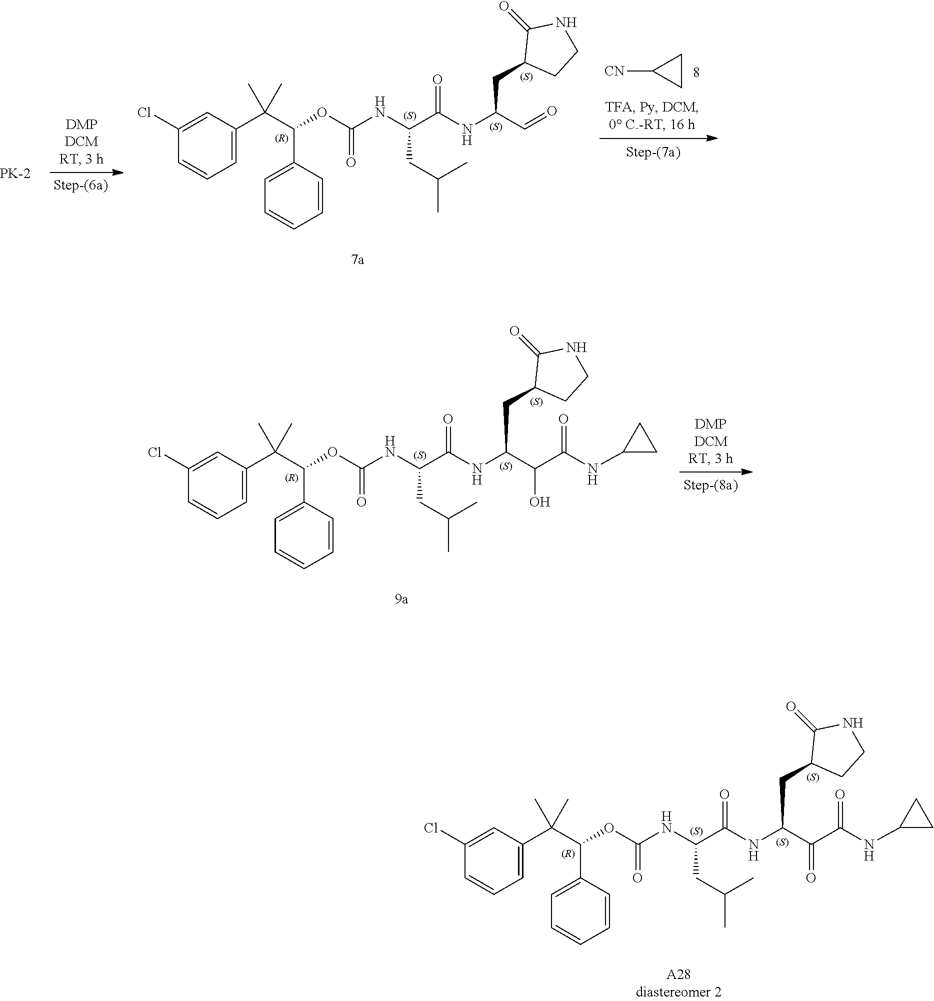

### Methyl ((2-(3-chlorophenyl)-2-methyl-1-phenylpropoxy) carbonyl)-L-leucinate (3)

To a stirred solution of 2-(3-chlorophenyl)-2-methyl-1-phenylpropan-1-ol (1) (12.5 g, 48.07 mmol), methyl (S)-2-amino-3,3-dimethylbutanoate HCl (10.47 g, 57.69 mmol) in DCM (50 mL) was added pyridine (38 mL, 3 vol) followed by triphosgene (7.1 g, 24.03 mmol) at 0° C., and the mixture was stirred at room temperature for 3 hours. The progress of the reaction was monitored by TLC and LCMS. The reaction mixture was quenched with 2N HCl (50 mL), and extracted with DCM (2×40 mL). The combined organic layer was dried over sodium sulfate, filtered and evaporated under reduced pressure. The residue was purified by reverse phase-high-performance liquid chromatography (RP-HPLC) (10% ABC:ACN) to afford methyl ((2-(3-chlorophenyl)-2-methyl-1-phenylpropoxy) carbonyl)-L-leucinate (3). TLC system: 20% Ethyl acetate in hexane Rf: 0.3; LCMS (ESI): m/z 454.48 [M+Na]⁺. Chiral HPLC data: PK-1 39% & PK-2 56%

### ((2-(3-Chlorophenyl)-2-methyl-1-phenylpropoxy) carbonyl)-L-leucine (4)

To a stirred solution of methyl ((2-(3-chlorophenyl)-2-methyl-1-phenylpropoxy) carbonyl)-L-leucinate (3) (7.2 g, 16.70 mmol) in THE (40 mL), water (40 mL) was added lithium hydroxide (1.4 g, 33.41 mmol) at 0° C. and the mixture was stirred at room temperature for 2 hours. The progress of the reaction was monitored by TLC and LCMS. The reaction mixture was completely evaporated under reduced pressure, and the residue was acidified with aq. 1N HCl solution up to pH˜3. The solution was extracted with ethyl acetate (2×40 mL), dried over sodium sulfate, and concentrated under reduced pressure to afford ((2-(3-chlorophenyl)-2-methyl-1-phenylpropoxy)carbonyl)-L-leucine (4). TLC system: 100% EtOAc Rf: 0.1; LCMS (ESI): m/z 440.47 [M+Na]⁺. Chiral HPLC data: PK-1 39% & PK-2 60%

### Methyl (2S)-2-((2S)-2-(((2-(3-chlorophenyl)-2-methyl-1-phenylpropoxy) carbonyl) amino)-4-methylpentanamido)-3-((S)-2-oxopyrrolidin-3-yl) propanoate (5)

To a stirred solution of ((2-(3-chlorophenyl)-2-methyl-1-phenylpropoxy)carbonyl)-L-leucine (4) (7.2 g, 16.78 mmol) DMF (30 mL) was added EDC·HCl (4.8 g, 25.17 mmol), HOBt (3.3 g, 25.17 mmol), DIPEA (8.7 mL, 50.35 mmol) and methyl (S)-2-amino-3-((S)-2-oxopyrrolidin-3-yl)propanoate hydrochloride (amine fragment-2) (3.7 g, 20.14 mmol) at 0° C. simultaneously, and the mixture was stirred at room temperature for 16 hours. The reaction mixture was diluted with ice water (80 mL), extracted with ethyl acetate (2×50 mL), dried over sodium sulfate and evaporated under reduced pressure. The residue was purified by RP-HPLC (10% ABC:ACN) to afford methyl (2S)-2-((2S)-2-(((2-(3-chlorophenyl)-2-methyl-1-phenylpropoxy)carbonyl)amino)-4-methylpentanamido)-3-((S)-2-oxopyrrolidin-3-yl)propanoate (5). TLC system: 10% Methanol in dichloromethane Rf: 0.3; LCMS (ESI): m/z 586.62 [M+H]⁺. Chiral HPLC data: PK-1 45% & PK-2 54%

### 2-(3-Chlorophenyl)-2-methyl-1-phenylpropyl ((S)-1-(((S)-1-hydroxy-3-((S)-2-oxopyrrolidin-3-yl)propan-2-yl)amino)-4-methyl-1-oxopentan-2-yl)carbamate (6)

To a stirred solution of methyl (2S)-2-((2S)-2-(((2-(3-chlorophenyl)-2-methyl-1-phenylpropoxy)carbonyl)amino)-4-methylpentanamido)-3-((S)-2-oxopyrrolidin-3-yl)propanoate (5) (4.4 g, 7.52 mmol) in DCM (20 mL) was added 2M LiBH₄ in THE (7.5 mL, 15.04 mmol) at 0° C. and the reaction mixture was stirred for 2 hours at 0° C. The progress of the reaction was monitored by TLC and LCMS. The reaction mixture was quenched with saturated ammonium chloride solution (30 mL) and extracted with DCM (2×30 mL). The organic layer was washed with brine solution (30 mL), dried over Na₂SO₄ and concentrated to afford 2-(3-chlorophenyl)-2-methyl-1-phenylpropyl ((S)-1-(((S)-1-hydroxy-3-((S)-2-oxopyrrolidin-3-yl)propan-2-yl)amino)-4-methyl-1-oxopentan-2-yl)carbamate (6). TLC system: 10% Methanol in dichloromethane Rf: 0.2; LCMS (ESI): m/z 558.9 [M+Na]⁺. Chiral HPLC data: PK-1 46% & PK-2 49%

### (S)-2-(3-Chlorophenyl)-2-methyl-1-phenylpropyl ((S)-1-(((S)-1-hydroxy-3-((S)-2-oxopyrrolidin-3-yl) propan-2-yl) amino)-4-methyl-1-oxopentan-2-yl)carbamate (PK-1) & (R)-2-(3-Chlorophenyl)-2-methyl-1-phenylpropyl ((S)-1-(((S)-1-hydroxy-3-((S)-2-oxopyrrolidin-3-yl)propan-2-yl)amino)-4-methyl-1-oxopentan-2-yl)carbamate (PK-2)

2-(3-chlorophenyl)-2-methyl-1-phenylpropyl ((S)-1-(((S)-1-hydroxy-3-((S)-2-oxopyrrolidin-3-yl)propan-2-yl)amino)-4-methyl-1-oxopentan-2-yl)carbamate (6) (4 g, 7.16 mmol) was separated via SFC to afford (S)-2-(3-chlorophenyl)-2-methyl-1-phenylpropyl((S)-1-(((S)-1-hydroxy-3-((S)-2-oxopyrrolidin-3-yl) propan-2-yl)amino)-4-methyl-1-oxopentan-2-yl)carbamate (PK-1) and (R)-2-(3-chlorophenyl)-2-methyl-1-phenylpropyl((S)-1-(((S)-1-hydroxy-3-((S)-2-oxopyrrolidin-3-yl)propan-2-yl)amino)-4-methyl-1-oxopentan-2-yl)carbamate (PK-2). TLC system: 5% Methanol in dichloromethane Rf: 0.3; LCMS (ESI): m/z 558.27 [M−OH]⁻. Chiral HPLC data: PK-1 99% Chiral HPLC data: PK-2 99%

### (S)-2-(3-Chlorophenyl)-2-methyl-1-phenylpropyl ((S)-4-methyl-1-oxo-1-(((S)-1-oxo-3-((S)-2-oxopyrrolidin-3-yl)propan-2-yl)amino)pentan-2-yl)carbamate (7)

To a stirred solution of (S)-2-(3-chlorophenyl)-2-methyl-1-phenylpropyl ((S)-1-(((S)-1-hydroxy-3-((S)-2-oxopyrrolidin-3-yl)propan-2-yl)amino)-4-methyl-1-oxopentan-2-yl)carbamate (PK-1) (700 mg, 1.25 mmol) in dichloromethane (15 mL) was added Dess-Martin periodinane (1 g, 2.51 mmol) at 0° C. and the mixture was stirred at room temperature for 3 hours. The reaction mixture was filtered through a Celite® pad and washed with ethyl acetate (25 mL), then washed with hypo solution (3×20 mL) followed by saturated NaHCO₃ solution (3×20 mL). The organic layer was dried over anhydrous Na₂SO₄, filtered, and concentrated to afford a residue that was purified by normal phase chromatography to afford (S)-2-(3-chlorophenyl)-2-methyl-1-phenylpropyl ((S)-4-methyl-1-oxo-1-(((S)-1-oxo-3-((S)-2-oxopyrrolidin-3-yl)propan-2-yl)amino)pentan-2-yl)carbamate (7). TLC system: 5% Methanol in dichloromethane Rf: 0.3; LCMS (ESI): m/z 556.45 (M+H)⁺. Chiral HPLC data: PK-1 82%

### (S)-2-(3-Chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (9)

To a stirred solution of (S)-2-(3-chlorophenyl)-2-methyl-1-phenylpropyl ((S)-4-methyl-1-oxo-1-(((S)-1-oxo-3-((S)-2-oxopyrrolidin-3-yl)propan-2-yl)amino)pentan-2-yl) carbamate (7) (550 mg, 0.98 mmol) was dissolved in DCM (15 mL) was added pyridine (0.54 mL, 3 vol) and isocyanocyclopropane (8) (0.13 ml, 1.98 mmol) sequentially at 0° C., and the mixture was stirred for 10 min. To this solution was added TFA (0.07 mL, 0.99 mmol) at 0° C., and the mixture was stirred at room temperature for 16 hours. The progress of the reaction was monitored by TLC and LCMS. The reaction mixture was quenched with ice water (20 mL) and extracted with dichloromethane (2×15 mL). The organic layer was washed with 1N HCl (3×15 mL) and brine solution (3×10 mL). The organic layer was dried over anhydrous Na₂SO₄ and evaporated under reduced pressure to afford (S)-2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (9). TLC system: 5% Methanol in dichloromethane Rf: 0.5; LCMS (ESI): m/z 641.5 [M+H]⁺. Chiral HPLC data: PK-1 49% & PK-2 33%

### (S)-2-(3-Chlorophenyl)-2-methyl-1-phenylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound A28 diastereomer 1)

To a stirred solution of (S)-2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (9) (350 mg, 0.54 mmol) in EtOAc (10 mL) was added Dess-Martin periodinane (463 mg, 1.09 mmol) at 0° C., and the mixture was stirred at room temperature for 3 hours. The progress of the reaction was monitored by TLC and LCMS. The reaction mixture was filtered through a Celite® pad and washed with ethyl acetate (25 mL), followed by hypo solution (3×20 mL) and saturated NaHCO₃ solution (3×20 mL). The organic layer was dried over anhydrous Na₂SO₄, filtered, and concentrated to afford a residue that was purified by RP-HPLC (10% ABC:ACN) to afford (S)-2-(3-chlorophenyl)-2-methyl-1-phenylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound A28 diastereomer 1). TLC system: 10% Methanol/Dichloromethane Rf: 0.4; LCMS (ESI): m/z 639.2 (M+H)⁺. Chiral HPLC data: PK-1 96%

### (R)-2-(3-Chlorophenyl)-2-methyl-1-phenylpropyl ((S)-4-methyl-1-oxo-1-(((S)-1-oxo-3-((S)-2-oxopyrrolidin-3-yl)propan-2-yl)amino)pentan-2-yl)carbamate (7a)

To a stirred solution of (R)-2-(3-chlorophenyl)-2-methyl-1-phenylpropyl ((S)-1-(((S)-1-hydroxy-3-((S)-2-oxopyrrolidin-3-yl)propan-2-yl)amino)-4-methyl-1-oxopentan-2-yl)carbamate (PK-2) (700 mg, 1.25 mmol) in dichloromethane (15 mL) was added Dess-Martin periodinane (1 g, 2.51 mmol) at 0° C., and the mixture was stirred at room temperature for 3 hours. The reaction mixture was filtered through a Celite® pad and washed with ethyl acetate (25 mL), hypo solution (3×20 mL), and saturated NaHCO₃ solution (3×20 mL). The organic layer was dried over anhydrous Na₂SO₄, filtered, and concentrated to afford a reside that was purified by normal phase chromatography to afford (R)-2-(3-chlorophenyl)-2-methyl-1-phenylpropyl ((S)-4-methyl-1-oxo-1-(((S)-1-oxo-3-((S)-2-oxopyrrolidin-3-yl)propan-2-yl)amino)pentan-2-yl)carbamate (7a). TLC system: 10% Methanol in dichloromethane Rf: 0.5; LCMS (ESI): m/z 556.45 (M+H)⁺. Chiral HPLC data: PK-1 93%

### (R)-2-(3-chlorophenyl)-2-methyl-1-phenylpropyl ((S)-4-methyl-1-oxo-1-(((S)-1-oxo-3-((S)-2-oxopyrrolidin-3-yl)propan-2-yl)amino)pentan-2-yl)carbamate (9a)

To a stirred solution of (R)-2-(3-chlorophenyl)-2-methyl-1-phenylpropyl ((S)-4-methyl-1-oxo-1-(((S)-1-oxo-3-((S)-2-oxopyrrolidin-3-yl)propan-2-yl)amino)pentan-2-yl)carbamate (7a) (520 mg, 0.98 mmol) in DCM (15 mL) was added pyridine (0.75 mL, 3 vol) and isocyanocyclopropane (8) (0.13 mL, 1.98 mmol) sequentially at 0° C., and the mixture was stirred for 10 minutes. To this mixture was added TFA (0.07 mL, 0.99 mmol) at 0° C., and the mixture was stirred at room temperature for 16 hours. The progress of the reaction was monitored by TLC and LCMS. The reaction mixture was quenched with ice water (20 mL) and extracted with dichloromethane (2×15 mL). The organic layer was washed with 1N HCl (3×15 mL) and brine solution (3×10 mL), then dried over anhydrous Na₂SO₄ and evaporated under reduced pressure to afford (R)-2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (9a). TLC system: 5% Methanol in dichloromethane Rf: 0.5; LCMS (ESI): m/z 641.5 [M+H]⁺. Chiral HPLC data: PK-1 27% & PK-1 60%

### (R)-2-(3-Chlorophenyl)-2-methyl-1-phenylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound A28 diastereomer 2)

To a stirred solution of (R)-2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (9a) (350 mg, 0.54 mmol) in EtOAc (15 mL) was added Dess-Martin periodinane (463 mg, 1.09 mmol) at 0° C., and the mixture was stirred at room temperature for 3 hours. The progress of the reaction was monitored by TLC and LCMS. The reaction mixture was filtered through a Celite® pad and washed with ethyl acetate (25 mL), hypo solution (3×20 mL), and saturated NaHCO₃ solution (3×20 mL). The organic layer was dried over anhydrous Na₂SO₄, filtered, and concentrated to afford a residue that was purified by RP-HPLC (10% ABC:ACN) to afford (R)-2-(3-chlorophenyl)-2-methyl-1-phenylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound A28 diastereomer 2). TLC system: 10% Methanol/Dichloromethane Rf: 0.4; LCMS (ESI): m/z 639.2 (M+H)⁺. Chiral HPLC data: PK-1 97%

### Example 7: Synthesis of Compound A29

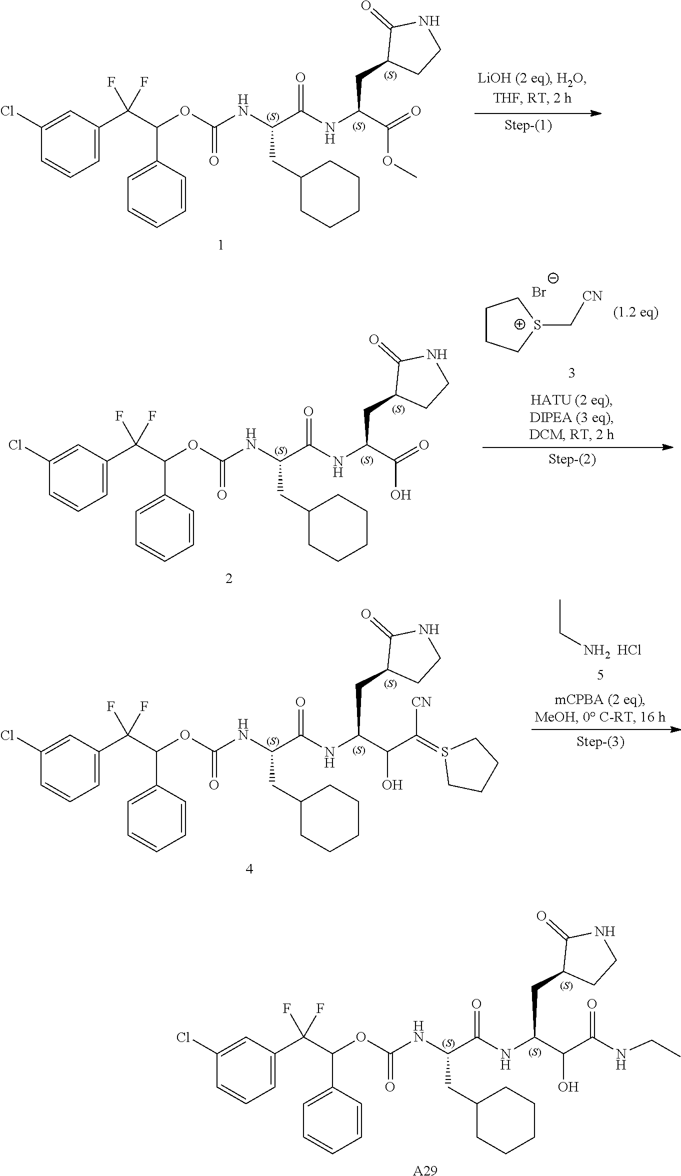

### (2S)-2-((2S)-2-(((2-(3-chlorophenyl)-2,2-difluoro-1-phenylethoxy)carbonyl)amino)-3-cyclohexylpropanamido)-3-((S)-2-oxopyrrolidin-3-yl)propanoic acid (2)

To a stirred solution of Methyl (2S)-2-((2S)-2-(((2-(3-chlorophenyl)-2,2-difluoro-1-phenylethoxy)carbonyl)amino)-3-cyclohexylpropanamido)-3-((S)-2-oxopyrrolidin-3-yl)propanoate (1) (1.8 mg, 2.838 mmol) in THE (30 mL) and water (15 mL) was added lithium hydroxide (357.6 mg, 8.516 mmol) at room temperature, and the mixture was stirred at room temperature for 2 hours. The progress of the reaction was monitored by TLC and LCMS. The reaction mixture was completely evaporated under reduced pressure, and the residue acidified with aq. 1N HCl solution up to pH˜2. The mixture was then extracted with ethyl acetate (2×20 mL), dried over sodium sulfate, and concentrated under reduced pressure to afford (2S)-2-((2S)-2-(((2-(3-chlorophenyl)-2,2-difluoro-1-phenylethoxy)carbonyl)amino)-3-cyclohexylpropanamido)-3-((S)-2-oxopyrrolidin-3-yl)propanoic acid (2). TLC system: 5% Methanol in dichloromethane Rf: 0.4; LCMS (ESI): m/z=620.60 [M+H]⁺

### 2-(3-Chlorophenyl)-2,2-difluoro-1-phenylethyl ((S)-1-(((S)-4-cyano-3-oxo-1-((S)-2-oxopyrrolidin-3-yl)-4-(tetrahydro-1|4-thiophen-1-ylidene)butan-2-yl)amino)-3-cyclohexyl-1-oxopropan-2-yl)carbamate (8)

To a stirred solution of (2S)-2-((2S)-2-(((2-(3-chlorophenyl)-2,2-difluoro-1-phenylethoxy)carbonyl)amino)-3-cyclohexylpropanamido)-3-((S)-2-oxopyrrolidin-3-yl)propanoic acid (2) (1.0 g, 1.612 mmol) in DCM (30 mL) was added HATU (903.09 mg, 3.225 mmol), DIPEA (0.89 mL, 4.838 mmol) and 1-(cyan methyl)tetra hydro-1H-thiophen-1-iumbromide (3) (503.4 mg, 2.419 mmol) at 0° C. simultaneously, and the mixture was stirred at room temperature for 2 hours. The reaction mixture was diluted with ice water (50 mL), extracted with dichloromethane (2×50 mL), dried over sodium sulfate and evaporated under reduced pressure. The residue was purified by combi-flash NP, then eluted at 5% methanol in dichloromethane to afford 2-(3-chlorophenyl)-2,2-difluoro-1-phenylethyl ((S)-1-(((S)-4-cyano-3-oxo-1-((S)-2-oxopyrrolidin-3-yl)-4-(tetrahydro-1|4-thiophen-1-ylidene)butan-2-yl)amino)-3-cyclohexyl-1-oxopropan-2-yl)carbamate (4). TLC system: 5% Methanol in dichloromethane Rf: 0.3; LCMS (ESI): m/z 729.61 [M+H]⁺

### 2-(3-Chlorophenyl)-2,2-difluoro-1-phenylethyl ((S)-3-cyclohexyl-1-(((S)-4-(ethylamino)-3,4-dioxo-1-((S)-2-oxopyrrolidin-3-yl)butan-2-yl)amino)-1-oxopropan-2-yl)carbamate (6)

To a stirred solution of 2-(3-chlorophenyl)-2,2-difluoro-1-phenylethyl ((S)-1-(((S)-4-cyano-3-oxo-1-((S)-2-oxopyrrolidin-3-yl)-4-(tetrahydro-1|4-thiophen-1-ylidene)butan-2-yl)amino)-3-cyclohexyl-1-oxopropan-2-yl)carbamate (4) (400 mg, 0.548 mmol) in methanol (30 mL) was added m-CPBA (189.13 mg, 1.096 mmol) at 0° C. and the reaction mixture was stirred for 2 hours at 0° C. To this was added ethylamine HCl (5) (1.34 g, 16.454 mL) and DIPEA (3.03 mL, 16.454 mmol) and the mixture was stirred at room temperature for 16 hours. The progress of the reaction was monitored by TLC and LCMS. The reaction mixture was diluted with dichloromethane and washed with saturated NaHCO₃ solution (2×20 mL). The organic layer was washed with brine solution (30 mL), dried over Na₂SO₄ and concentrated to afford a residue that was purified by prep HPLC to afford 2-(3-Chlorophenyl)-2,2-difluoro-1-phenylethyl ((S)-3-cyclohexyl-1-(((S)-4-(ethylamino)-3,4-dioxo-1-((S)-2-oxopyrrolidin-3-yl)butan-2-yl)amino)-1-oxopropan-2-yl)carbamate (Compound A29). TLC system: 10% Methanol in dichloromethane Rf: 0.3; LCMS (ESI): m/z 675.4 [M+H]⁺

### Example 8: Synthesis of Compounds A107 and A110

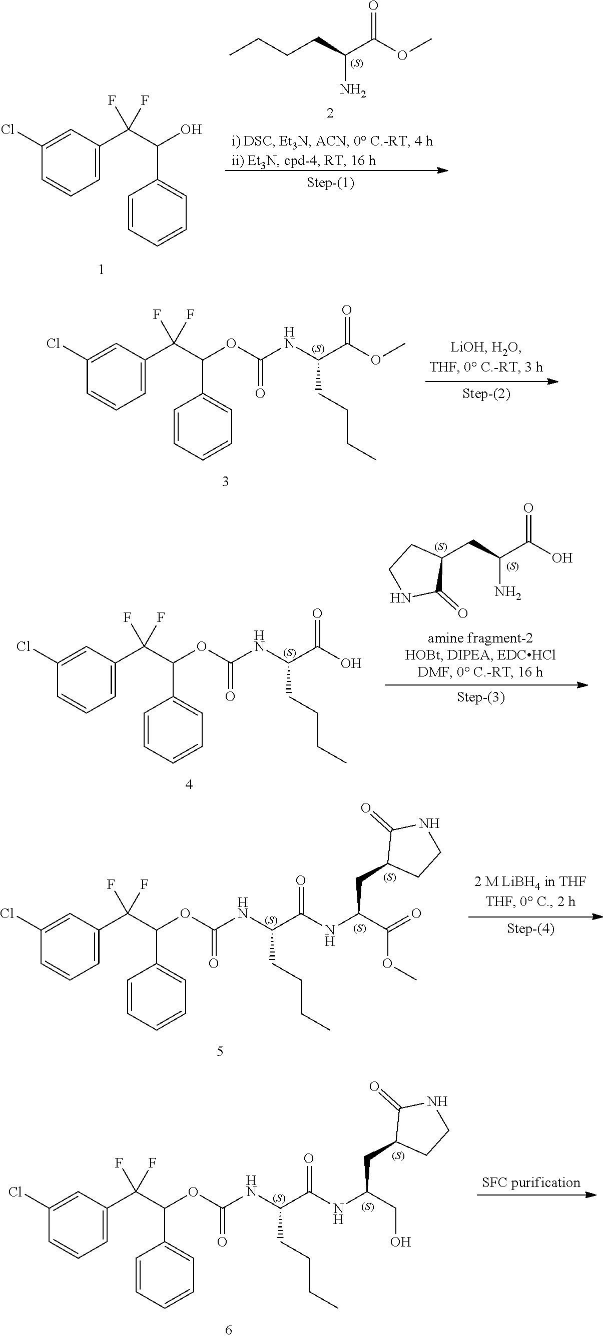

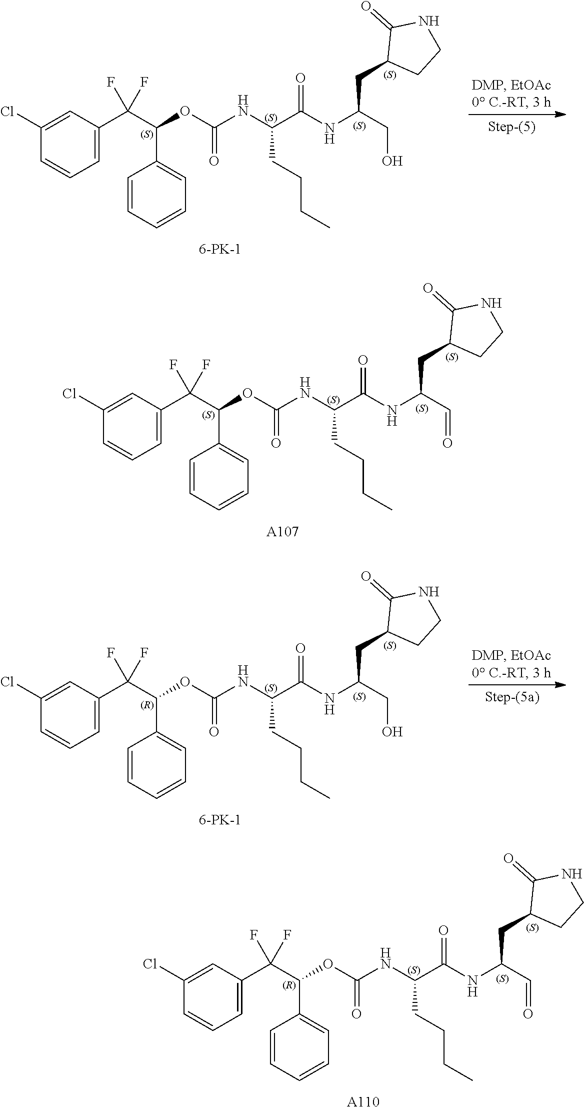

### Methyl (2S)-2-(((2-(3-chlorophenyl)-2,2-difluoro-1 phenylethoxy)carbonyl)amino)hexanoate (3)

To a stirred solution of 2-(3-chlorophenyl)-2,2-difluoro-1-phenylethan-1-ol (1) (50 g, 186.56 mmol) in ACN (500 mL) was added N,N′disuccinamidyl carbonate (119.4 g, 466.41 mmol), followed by Et₃N (80.7 mL, 559.74 mmol) at 0° C. and stirred at room temperature for 4 h. The progress of the reaction was monitored by TLC. The reaction mass was used directly in the subsequent reaction.

In another RB flask, methyl (S)-2-aminohexanoate (2) (70 g, 171.96 mmol) was dissolved in ACN (350 mL), and added Et₃N (46 mL, 318.21 mmol). The reaction mixture was stirred for 5 min, then added above prepared reaction mass drop-wise and the reaction mixture stirred at room temperature for 16 h. Reaction mixture was quenched with ice water (200 mL) and extracted with ethyl acetate (2×100 mL). The combined organic layer was washed with brine (100 mL), dried over sodium sulfate and evaporated under reduced pressure to afford material, which was purified by silica gel (200-300 mesh) column chromatography by eluting with 10% ethyl acetate in pet ether to afford methyl (2S)-2-(((2-(3-chlorophenyl)-2,2-difluoro-1 phenylethoxy) carbonyl)amino)hexanoate (3). TLC system: 10% Ethyl acetate in hexane Rf: 0.3 LCMS (ESI): m/z 440.13 [M+H]⁺

### (2S)-2-(((2-(3-chlorophenyl)-2,2-difluoro-1-phenylethoxy)carbonyl)amino)hexanoic acid (4)

To a stirred solution of methyl (2S)-2-(((2-(3-chlorophenyl)-2,2-difluoro-1 phenylethoxy) carbonyl)amino)hexanoate (3) (40 g, 90.1 mmol) in THE (200 mL): water (200 mL) was added lithium hydroxide (9.56 g, 227.71 mmol) at 0° C. and stirred at room temperature for 3 h. The progress of the reaction was monitored by TLC and LC-MS. The reaction mixture was concentrated and acidified with aq. 1N HCl up to pH˜2 and extracted with ethyl acetate (2×200 mL), dried over sodium sulfate, concentrated under reduced pressure to afford material, which was purified by silica gel (230-300 mesh) column chromatography by eluting using 30% ethyl acetate in pet ether to afford (2S)-2-(((2-(3-chlorophenyl)-2,2-difluoro-1-phenylethoxy)carbonyl)amino)hexanoic acid (4). TLC system: 5% MeOH: DCM Rf: 0.3 LCMS (ESI): m/z=448.26 [M+Na]⁺

### Methyl (2S)-2-((2S)-2-(((2-(3-chlorophenyl)-2,2-difluoro-1-phenylethoxy)carbonyl)amino)hexanamido)-3-((S)-2-oxopyrrolidin-3-yl)propanoate (5)

To a stirred solution of (2S)-2-(((2-(3-chlorophenyl)-2,2-difluoro-1-phenylethoxy) carbonyl)amino)hexanoic acid (4) (20 g, 47.05 mmol) in DMF (100 mL) was added EDC·HCl (13.72 g, 70.4 mmol), HOBt (9.5 g, 70.4 mmol), DIPEA (25 mL, 56.446 mmol) and methyl (S)-2-amino-3-((S)-2-oxopyrrolidin-3-yl)propanoate (amine fragment-2) (15.67 g, 70.4 mmol) at 0° C. The reaction mixture was allowed to stir at room temperature for 16 h. To the reaction mixture, ice water (200 mL) was added, extracted with ethyl acetate (2×150 mL) and washed with ice cold water (2×100 mL). The combined organic layer was dried over sodium sulfate and evaporated under reduced pressure to afford compound. The residue was purified by silica gel column chromatography by eluting with 100% ethyl acetate to afford methyl (2S)-2-((2S)-2-(((2-(3-chlorophenyl)-2,2-difluoro-1-phenylethoxy)carbonyl)amino)hexanamido)-3-((S)-2-oxopyrrolidin-3-yl)propanoate (5). TLC system: 5% Methanol in dichloromethane Rf: 0.3 LCMS (ESI): m/z 594.3 [M+H]⁺

### 2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6)

To a stirred solution of methyl (2S)-2-((2S)-2-(((2-(3-chlorophenyl)-2,2-difluoro-1-phenylethoxy)carbonyl)amino)hexanamido)-3-((S)-2-oxopyrrolidin-3-yl)propanoate (5) (19 g, 32.04 mmol) in THE (190 mL) was added 2M LiBH₄ in THE (32 mL, 64.08 mmol) at 0° C. and stirred for 2 h. The progress of the reaction was monitored by TLC and LC-MS. The reaction mixture was quenched with saturated NH₄Cl solution (250 mL) and extracted with ethyl acetate (2×200 mL), dried over sodium sulfate, concentrated under reduced pressure to afford compound. The compound was purified by reverse phase column chromatography to afford 2-(3-chlorophenyl)-2,2-difluoro-1-phenylethyl((S)-1-(((S)-1-hydroxy-3-((S)-2-oxopyrrolidin-3-yl)propan-2-yl)amino)-1-oxohexan-2-yl)carbamate (6). TLC system: 5% Methanol in dichloromethane Rf: 0.4 LCMS (ESI): m/z=566.2 [M+H]⁺

### (S)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-1) & (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-2)

15 g of 2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6) was purified by Chiral SFC to afford ((S)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-1) and (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-2). TLC system: 5% Methanol in dichloromethane Rf: 0.4 LCMS (ESI): m/z=566.2 [M+H]⁺

### (S)-2-(3-chlorophenyl)-2,2-difluoro-1-phenylethyl ((S)-1-oxo-1-(((S)-1-oxo-3-((S)-2-oxopyrrolidin-3-yl)propan-2-yl)amino)hexan-2-yl)carbamate (A107)

To a stirred solution of (S)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-1) (4 g, 7.064 mmol) in ethyl acetate (80 mL) was added Dess-Martin periodinane (6 g, 14.132 mmol) at 0° C. and stirred at RT for 3 h. Reaction mixture was diluted with ethyl acetate (200 mL) and washed with sat. Hypo solution (3×100 mL), sat. NaHCO₃ solution (3×100 mL) and brine (2×50 mL). Organic layer was dried over anhydrous Na₂SO₄, filtered and concentrated to afford compound. The compound was purified by reverse phase column chromatography to afford (S)-2-(3-chlorophenyl)-2,2-difluoro-1-phenylethyl ((S)-1-oxo-1-(((S)-1-oxo-3-((S)-2-oxopyrrolidin-3-yl)propan-2-yl)amino)hexan-2-yl)carbamate (A107). TLC system: 5% Methanol in dichloromethane Rf: 0.3 LCMS (ESI): m/z 564.3 [M+H]⁺

### (R)-2-(3-chlorophenyl)-2,2-difluoro-1-phenylethyl ((S)-1-oxo-1-(((S)-1-oxo-3-((S)-2-oxopyrrolidin-3-yl)propan-2-yl)amino)hexan-2-yl)carbamate (A110)

To a stirred solution of (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxohexan-2-yl)carbamate (6-PK-2) (6 g, 10.64 mmol) was dissolved in ethyl acetate (40 mL) was added Dess-Martin periodinane (6 g, 14.132 mmol) at 0° C. and stirred at RT for 3 h. Reaction mixture was diluted with ethyl acetate (200 mL) and washed with sat. Hypo solution (3×100 mL), sat. NaHCO₃ solution (3×100 mL) and brine (2×50 mL). Organic layer was dried over anhydrous Na₂SO₄, filtered and concentrated to afford compound. The compound was purified by reverse phase column chromatography to afford (R)-2-(3-chlorophenyl)-2,2-difluoro-1-phenylethyl ((S)-1-oxo-1-(((S)-1-oxo-3-((S)-2-oxopyrrolidin-3-yl)propan-2-yl)amino)hexan-2-yl)carbamate (A110). TLC system: 5% Methanol in dichloromethane Rf: 0.3 LCMS (ESI): m/z 564.2 [M+H]⁺

### Example 9: Synthesis of Compound A122

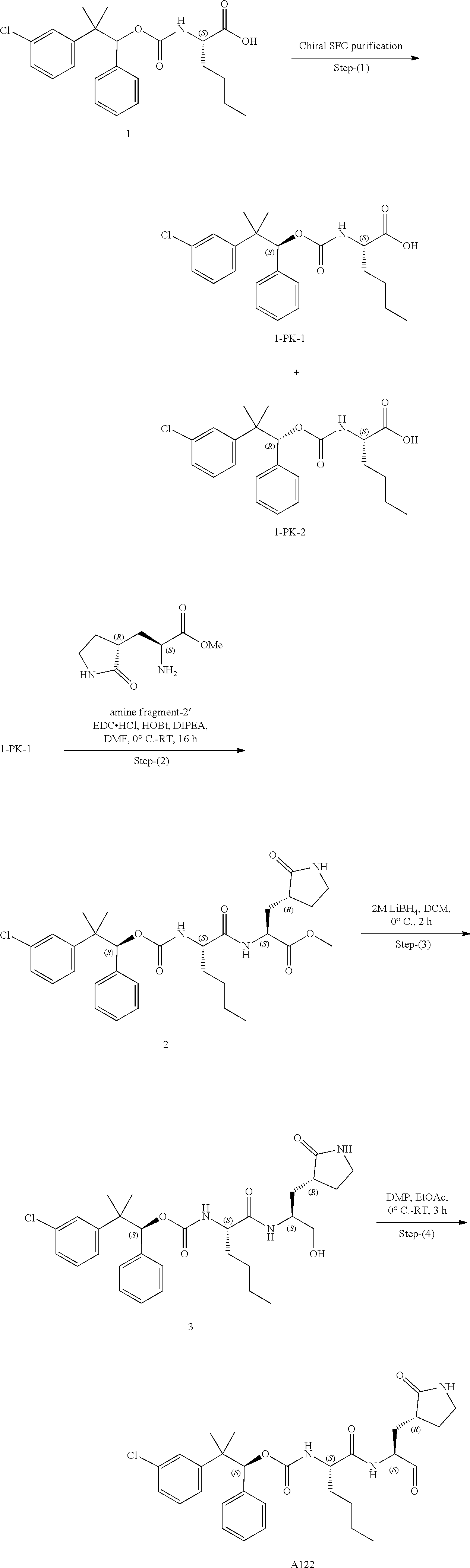

### (S)-2-((((S)-2-(3-chlorophenyl)-2-methyl-1-phenylpropoxy)carbonyl)amino)hexanoic acid (1-PK-1) & (S)-2-((((R)-2-(3-chlorophenyl)-2-methyl-1-phenylpropoxy)carbonyl)amino)hexanoic acid (1-PK-2)

10 g of (2S)-2-(((2-(3-chlorophenyl)-2-methyl-1-phenylpropoxy)carbonyl) amino) hexanoic acid (1) was purified by Chiral SFC to afford (S)-2-((((S)-2-(3-chlorophenyl)-2-methyl-1-phenylpropoxy)carbonyl)amino)hexanoic acid (1-PK-1) (4.2 g, 5.99 mmol) and (S)-2-((((R)-2-(3-chlorophenyl)-2-methyl-1-phenylpropoxy)carbonyl)amino)hexanoic acid (1-PK-2). TLC system: 10% MeOH in DCM Rf: 0.5 LCMS (ESI): m/z 440.2 [M+Na]⁺

### methyl (S)-2-((S)-2-((((S)-2-(3-chlorophenyl)-2-methyl-1-phenylpropoxy)carbonyl)amino)hexanamido)-3-((R)-2-oxopyrrolidin-3-yl)propanoate (2)

To a stirred solution of (S)-2-((((S)-2-(3-chlorophenyl)-2-methyl-1-phenylpropoxy) carbonyl)amino)hexanoic acid (1-PK-1) (4 g, 9.6 mmol) in DMF (20 mL) were added EDC·HCl (2.7 g, 13.8 mmol), HOBt (1.86 g, 13.8 mmol), DIPEA (5 mL, 27.6 mmol) and methyl (S)-2-amino-3-((S)-2-oxopyrrolidin-3-yl)propanoate hydrochloride (amine fragment-2′) (2.56 g, 13.8 mmol) at 0° C. The reaction mixture was allowed to stir at room temperature for 16 h. After completion of the reaction by TLC, the reaction mixture was quenched with ice water (80 mL) and extracted with ethyl acetate (2×150 mL). The combined organic layer was washed with brine (2×50 mL), dried over sodium sulfate and evaporated under reduced pressure. The material was purified by combi-flash (normal phase), compound eluted at 5% methanol in dichloromethane to afford methyl (S)-2-((S)-2-((((S)-2-(3-chlorophenyl)-2-methyl-1-phenyl propoxy)carbonyl)amino)hexanamido)-3-((R)-2-oxopyrrolidin-3-yl)propanoate (2). TLC system: 100% EtOAc Rf: 0.4 LCMS (ESI): m/z 602.6 [M+H]⁺

### (S)-2-(3-chlorophenyl)-2-methyl-1-phenylpropyl ((S)-1-(((S)-1-hydroxy-3-((R)-2-oxopyrrolidin-3-yl)propan-2-yl)amino)-1-oxohexan-2-yl)carbamate (3)

To a stirred solution of methyl (S)-2-((S)-2-((((S)-2-(3-chlorophenyl)-2-methyl-1-phenylpropoxy)carbonyl)amino)hexanamido)-3-((R)-2-oxopyrrolidin-3-yl)propanoate (2) (4 g, 6.83 mmol) in DCM (20 mL) was added 2M LiBH₄ in THE (6.8 mL, 13.6 mmol) at 0° C. and the reaction mixture stirred for 2 h at 0° C. The progress of the reaction was monitored by TLC. The reaction mixture was quenched with sat. Ammonium chloride solution (20 mL) and extracted with DCM (2×50 mL). The combined organic layer was washed with brine (30 mL), dried over Na₂SO₄ and concentrated to afford (S)-2-(3-chlorophenyl)-2-methyl-1-phenylpropyl ((S)-1-(((S)-1-hydroxy-3-((R)-2-oxopyrrolidin-3-yl)propan-2-yl)amino)-1-oxohexan-2-yl)carbamate (3). TLC system: 5% MeOH in DCM Rf: 0.4 LCMS (ESI): m/z 558.3 [M+H]⁺

### (S)-2-(3-Chlorophenyl)-2-methyl-1-phenylpropyl ((S)-1-oxo-1-(((S)-1-oxo-3-((R)-2-oxopyrrolidin-3-yl)propan-2-yl)amino)hexan-2-yl)carbamate (A122)

To a stirred solution of (S)-2-(3-Chlorophenyl)-2-methyl-1-phenylpropyl ((S)-1-oxo-1-(((S)-1-oxo-3-((R)-2-oxopyrrolidin-3-yl)propan-2-yl)amino)hexan-2-yl)carbamate (1-PK-1) (3.5 g, 6.3 mmol) in ethyl acetate (50 mL) was added Dess-Martin periodinane (5.3 g, 12.5 mmol) at 0° C. and stirred at RT for 3 h. Reaction mixture was diluted with ethyl acetate (100 mL) and washed with sat. Hypo solution (3×50 mL), sat. NaHCO₃ solution (3×50 mL) and brine (1×50 mL). Organic layer was dried over anhydrous Na₂SO₄, filtered and concentrated to afford compound. The compound was purified by reverse phase combi-flash chromatography to afford (S)-2-(3-chlorophenyl)-2-methyl-1-phenylpropyl ((S)-1-oxo-1-(((S)-1-oxo-3-((R)-2-oxopyrrolidin-3-yl)propan-2-yl)amino)hexan-2-yl)carbamate (A122). TLC system: 5% Methanol in dichloromethane Rf: 0.5 LCMS (ESI): m/z 556.3 [M+H]⁺

### Example 10: Synthesis of Compounds A164 and A165

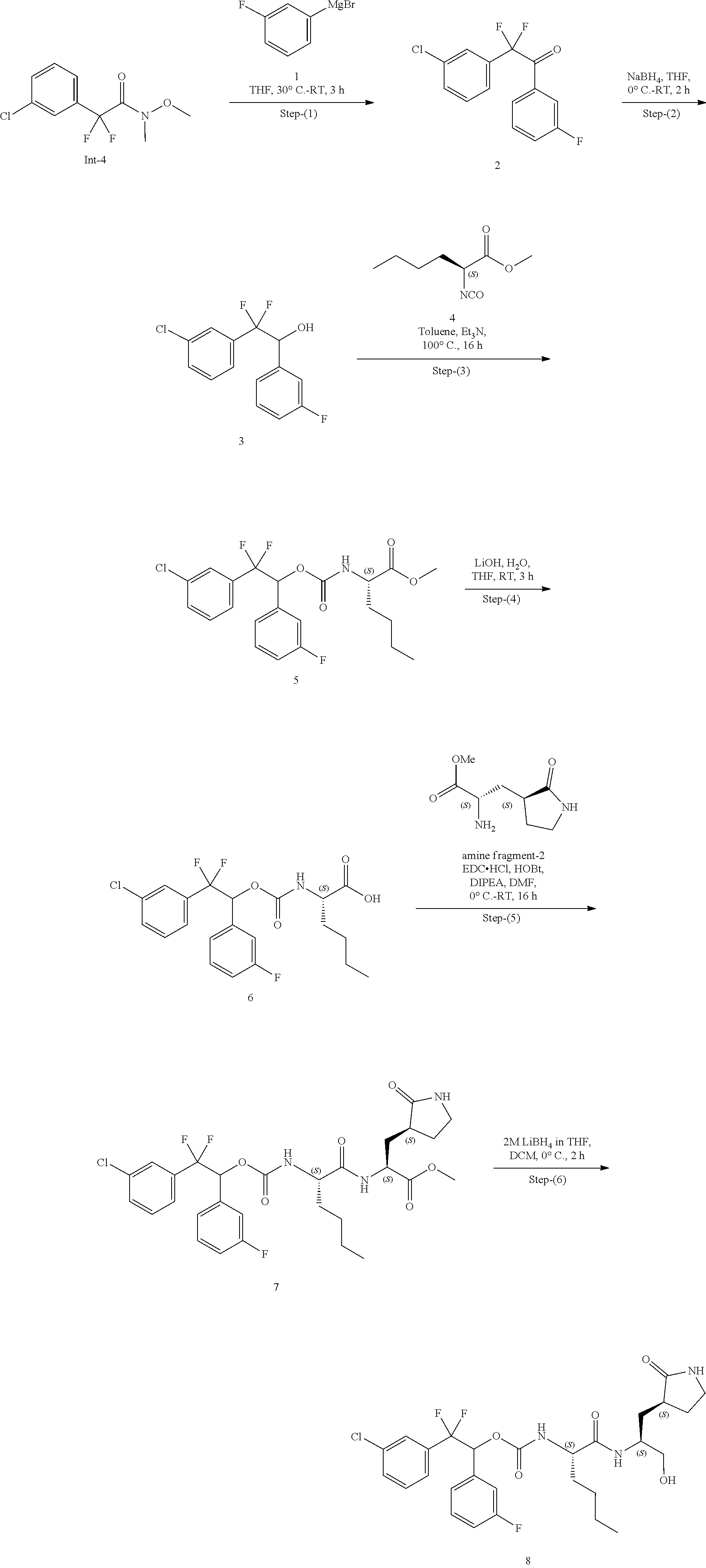

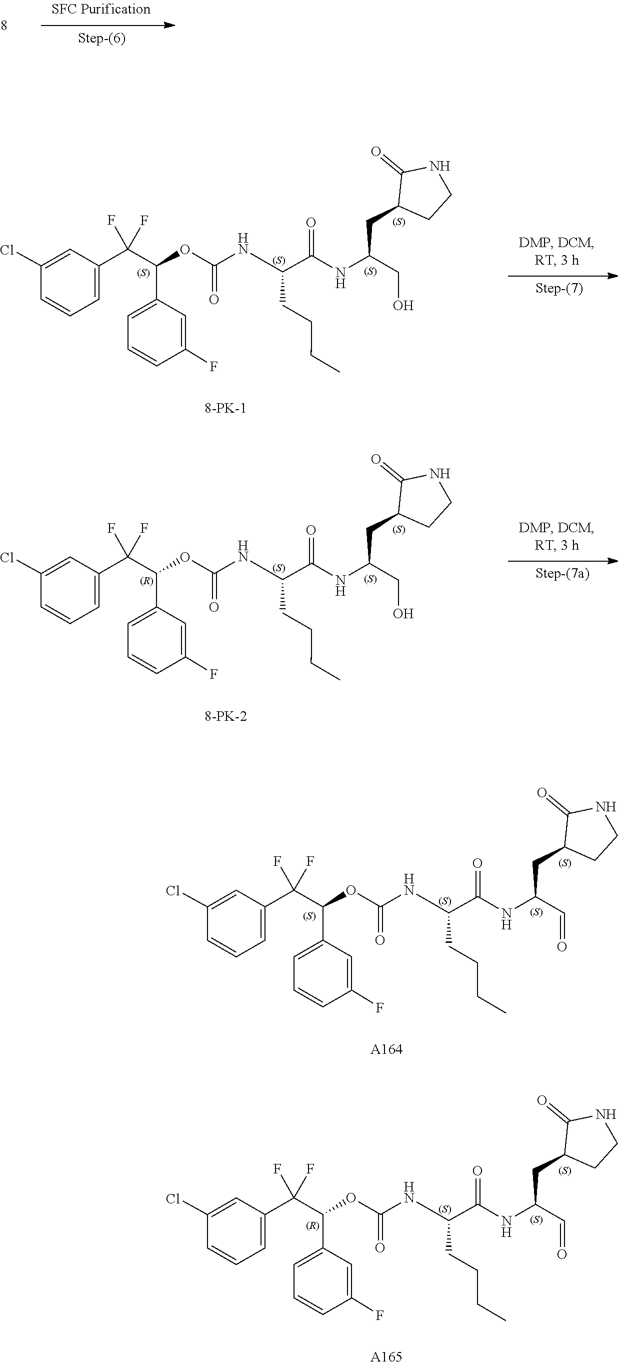

### 2-(3-Chlorophenyl)-2, 2-difluoro-1-(3-fluorophenyl) ethan-1-one (2)

To a stirred solution of (3-fluorophenyl)magnesium bromide (1) (freshly prepared from Mg (4.3 g, 258.49 mmol), 1-bromo-3-fluorobenzene (15 g, 86.16 mmol), 1,2-dibromoethane (3 mL) in diethyl ether at 0° C. was added a solution of 2-(3-chlorophenyl)-2,2-difluoro-N-methoxy-N-methylacetamide (15 g, 60.24 mmol) in THF (45 mL) slowly drop wise at 0° C. and stirred at RT for 3 h. The progress of the reaction was monitored by TLC. Reaction mixture was quenched with saturated ammonium chloride solution (500 mL) and extracted with ethyl acetate (2×700 mL). Combined organic layer was dried over anhydrous sodium sulfate and concentrated under reduced pressure. Obtained residue was purified by silica gel column chromatography by eluting with 5% ethyl acetate in hexane to afford 2-(3-chlorophenyl)-2,2-difluoro-1-(3-fluorophenyl)ethan-1-one (2). TLC system: 10% Ethyl acetate in hexane Rf: 0.3

### 2-(3-Chlorophenyl)-2, 2-difluoro-1-(3-fluorophenyl) ethan-1-ol (3)

To a stirred solution of 2-(3-chlorophenyl)-2,2-difluoro-1-(3-fluorophenyl)ethan-1-one (2) (9 g, 31.69 mmol) in THF (100 mL) wad added sodiumborohydride (3.59 g, 95.07 mmol) slowly portion wise at 0° C. and stirred at RT for 2 h. Reaction progress was monitored by TLC. After completion of starting material, reaction mixture was quenched with ice water and evaporated under reduced pressure to remove THF. Then added 1N HCl (150 mL) and extracted with ethyl acetate (2×100 mL). Combined organic layer was washed with water (150 mL), brine solution (150 mL), dried over sodium sulfate and evaporated under reduced pressure to afford 2-(3-chlorophenyl)-2,2-difluoro-1-(3-fluorophenyl)ethan-1-ol (3). TLC system: 10% Ethyl acetate in hexane Rf: 0.3

### Methyl (2S)-2-(((2-(3-chlorophenyl)-2, 2-difluoro-1-(3-fluorophenyl)ethoxy)carbonyl) amino)hexanoate (5)

To a stirred solution of 2-(3-chlorophenyl)-2,2-difluoro-1-(3-fluorophenyl)ethan-1-ol (3) (6.4 g, 22.37 mmol) in toluene (30 mL) was added methyl (S)-2-isocyanatohexanoate (9.2 g, 44.75 mmol), followed by triethylamine (9.4 mL, 67.13 mmol) at room temperature and heated to 100° C. for 16 h. The progress of the reaction was monitored by TLC. Reaction mixture was evaporated under reduced pressure and residue was purified by silica gel column chromatography by eluting with 6-7% ethyl acetate in hexane to afford methyl (2S)-2-(((2-(3-chlorophenyl)-2,2-difluoro-1-(3-fluorophenyl)ethoxy)carbonyl)amino)hexanoate (5). TLC system: 10% Ethyl acetate in hexane Rf: 0.5 LCMS (ESI): m/z 458.4 [M+H]⁺

### (2S)-2-(((2-(3-chlorophenyl)-2, 2-difluoro-1-(3-fluorophenyl) ethoxy) carbonyl) amino) hexanoic acid (6)

To a stirred solution of methyl (2S)-2-(((2-(3-chlorophenyl)-2,2-difluoro-1-(3-fluorophenyl)ethoxy)carbonyl)amino)hexanoate (5) (9.7 g, 21.22 mmol) in THF (50 mL) and water (15 mL) was added LiOH·H₂O (1.7 g, 42.45 mmol) at RT and stirred at room temperature for 3 h. The progress of the reaction was monitored by TLC. After completion of the reaction, the reaction mixture was concentrated under reduced pressure to remove THF. Obtained residue was acidified with aq. 1N HCl solution up to pH˜2 and extracted with ethyl acetate (2×100 mL). Combined organic layer was dried over anhydrous sodium sulfate and concentrated under reduced pressure to afford (2S)-2-(((2-(3-chlorophenyl)-2,2-difluoro-1-(3-fluorophenyl) ethoxy)carbonyl)amino)hexanoic acid (6). TLC system: 50% Ethyl acetate in hexane Rf: 0.1 LCMS (ESI): m/z=424.2 [M−19]⁺

### Methyl (2S)-2-((2S)-2-(((2-(3-chlorophenyl)-2, 2-difluoro-1-(3-fluorophenyl) ethoxy) carbonyl) amino) hexanamido)-3-((S)-2-oxopyrrolidin-3-yl) propanoate (7)

To a stirred solution of (2S)-2-(((2-(3-chlorophenyl)-2,2-difluoro-1-(3-fluorophenyl)ethoxy)carbonyl)amino)hexanoic acid (6) (4.0 g, 9.02 mmol) in DMF (20 mL) was added EDC·HCl (2.58 g, 13.54 mmol), HOBt (1.82 g, 13.54 mmol), DIPEA (4.85 mL, 27.08 mmol) and methyl (S)-2-amino-3-((S)-2-oxopyrrolidin-3-yl)propanoate (amine fragment-2) (2.0 g, 10.83 mmol) at 0° C. simultaneously and stirred at room temperature for 16 h. After completion of the reaction by TLC, reaction mixture was diluted with ice water (100 mL) and extracted with ethyl acetate (2×150 mL). Combined organic layer was washed with ice cold water (2×50 mL), dried over anhydrous sodium sulfate and evaporated under reduced pressure. Obtained residue was purified by silica gel column chromatography by eluting with 100% ethyl acetate to afford methyl (2S)-2-((2S)-2-(((2-(3-chlorophenyl)-2,2-difluoro-1-(3-fluorophenyl)ethoxy) carbonyl) amino)hexanamido)-3-((S)-2-oxopyrrolidin-3-yl)propanoate (7). TLC system: 10% Methanol in dichloromethane Rf: 0.4 LCMS (ESI): m/z 612.14 [M+H]⁺

### 2-(3-Chlorophenyl)-2, 2-difluoro-1-(3-fluorophenyl) ethyl ((S)-1-(((S)-1-hydroxy-3-((S)-2-oxopyrrolidin-3-yl) propan-2-yl) amino)-1-oxohexan-2-yl) carbamate (8)

To a stirred solution of methyl (2S)-2-((2S)-2-(((2-(3-chlorophenyl)-2,2-difluoro-1-(3-fluorophenyl)ethoxy)carbonyl)amino)hexanamido)-3-((S)-2-oxopyrrolidin-3-yl)propanoate (7) (2.2 g, 3.60 mmol) in dichloromethane (20 mL) was added 2M LiBH₄ in THE (3.6 mL, 7.20 mmol) slowly drop wise at 0° C. and stirred at same temperature for 2 h. The progress of the reaction was monitored by TLC. After completion of the reaction by TLC, reaction mixture was quenched with saturated aq·NH₄Cl solution and extracted with dichloromethane (2×150 mL). Combined organic layer was dried over anhydrous sodium sulfate and concentrated under reduced pressure to afford 2-(3-chlorophenyl)-2,2-difluoro-1-(3-fluorophenyl)ethyl ((S)-1-(((S)-1-hydroxy-3-((S)-2-oxopyrrolidin-3-yl)propan-2-yl)amino)-1-oxohexan-2-yl)carbamate (8). TLC system: 10% Methanol in dichloromethane Rf: 0.4 LCMS (ESI): m/z=584.8 [M+H]⁺

### (S)-2-(3-Chlorophenyl)-2-methyl-1-phenylpropyl ((S)-1-(((S)-1-hydroxy-3-((R)-2-oxopyrrolidin-3-yl)propan-2-yl)amino)-1-oxohexan-2-yl)carbamate (8-PK-1) & (R)-2-(3-Chlorophenyl)-2-methyl-1-phenylpropyl ((S)-1-(((S)-1-hydroxy-3-((R)-2-oxopyrrolidin-3-yl)propan-2-yl)amino)-1-oxohexan-2-yl)carbamate (8-PK-2)

2 g of 2-(3-Chlorophenyl)-2-methyl-1-phenylpropyl ((S)-1-(((S)-1-hydroxy-3-((R)-2-oxopyrrolidin-3-yl)propan-2-yl)amino)-1-oxohexan-2-yl)carbamate (3) was purified by chiral SFC to afford (S)-2-(3-Chlorophenyl)-2-methyl-1-phenylpropyl ((S)-1-oxo-1-(((S)-1-oxo-3-((R)-2-oxopyrrolidin-3-yl)propan-2-yl)amino)hexan-2-yl)carbamate (3-PK-1) (1 g, 2.71 mmol) and (R)-2-(3-Chlorophenyl)-2-methyl-1-phenylpropyl ((S)-1-oxo-1-(((S)-1-oxo-3-((R)-2-oxopyrrolidin-3-yl)propan-2-yl)amino)hexan-2-yl)carbamate (3-PK-2). TLC system: 10% Methanol in DCM Rf: 0.4 LCMS (ESI): m/z 584.57 [M+H]⁺

### (S)-2-(3-chlorophenyl)-2,2-difluoro-1-(3-fluorophenyl)ethyl ((S)-1-oxo-1-(((S)-1-oxo-3-((S)-2-oxopyrrolidin-3-yl)propan-2-yl)amino)hexan-2-yl)carbamate (A164)

To a stirred solution of (S)-2-(3-chlorophenyl)-2,2-difluoro-1-(3-fluorophenyl)ethyl ((S)-1-(((S)-1-hydroxy-3-((S)-2-oxopyrrolidin-3-yl)propan-2-yl)amino)-1-oxohexan-2-yl)carbamate (8) (450 mg, 0.77 mmol) in ethyl acetate (20 mL) was added Dess-Martin periodinane (981 mg, 2.31 mmol) at 0° C. and stirred at RT for 3 h. After completion of the reaction by TLC, reaction mixture was filtered through diatomaceous earth pad and washed with ethyl acetate (20 mL). Obtained filtrate was washed with sat. Hypo solution (3×20 mL), sat. NaHCO₃ solution (3×20 mL) and brine (1×20 mL). Organic layer was separated, dried over anhydrous Na₂SO₄, filtered and concentrated under educed pressure. Obtained compound was purified by trituration with DEE/n-Pentane to afford (S)-2-(3-chlorophenyl)-2,2-difluoro-1-(3-fluorophenyl)ethyl ((S)-1-oxo-1-(((S)-1-oxo-3-((S)-2-oxopyrrolidin-3-yl)propan-2-yl)amino)hexan-2-yl)carbamate (A164). TLC system: 5% Methanol in dichloromethane Rf: 0.4 LCMS (ESI): m/z 582.76 [M+H]⁺

### (R)-2-(3-chlorophenyl)-2,2-difluoro-1-(3-fluorophenyl)ethyl ((S)-1-oxo-1-(((S)-1-oxo-3-((S)-2-oxopyrrolidin-3-yl)propan-2-yl)amino)hexan-2-yl)carbamate (A165)

To a stirred solution of (R)-2-(3-chlorophenyl)-2,2-difluoro-1-(3-fluorophenyl)ethyl ((S)-1-(((S)-1-hydroxy-3-((S)-2-oxopyrrolidin-3-yl)propan-2-yl)amino)-1-oxohexan-2-yl)carbamate (8) (450 mg, 0.77 mmol) in ethyl acetate (20 mL) was added Dess-Martin periodinane (981 mg, 2.31 mmol) at 0° C. and stirred at RT for 3 h. After completion of the reaction by TLC, reaction mixture was filtered through diatomaceous earth pad and washed with ethyl acetate (20 mL). Obtained filtrate was washed with sat. Hypo solution (3×20 mL), sat. NaHCO₃ solution (3×20 mL) and brine (1×20 mL). Organic layer was separated, dried over anhydrous Na₂SO₄, filtered and concentrated under reduced pressure. Obtained compound was purified by trituration with DEE/n-Pentane to afford (S)-2-(3-chlorophenyl)-2,2-difluoro-1-(3-fluorophenyl)ethyl ((S)-1-oxo-1-(((S)-1-oxo-3-((S)-2-oxopyrrolidin-3-yl)propan-2-yl)amino)hexan-2-yl)carbamate (A165). TLC system: 5% Methanol in dichloromethane Rf: 0.4 LCMS (ESI): m/z 582.7 [M+H]⁺

### Example 11: Synthesis of Compound A173

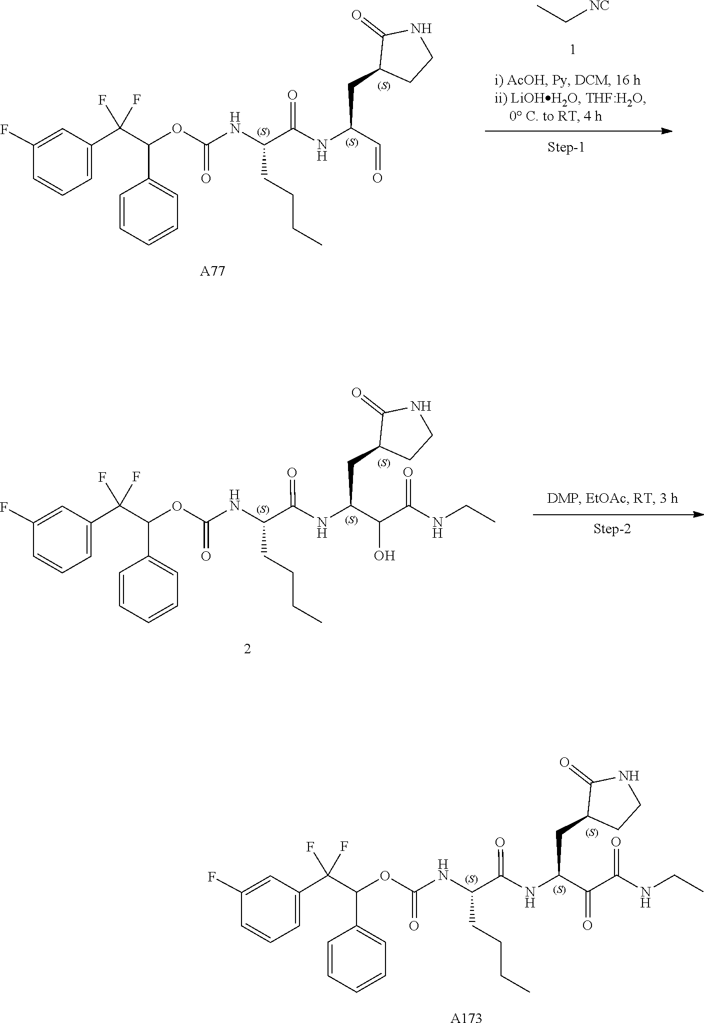

### 2,2-Difluoro-2-(3-fluorophenyl)-1-phenylethyl ((2S)-1-(((2S)-4-(ethylamino)-3-hydroxy-4-oxo-1-((R)-2-oxopyrrolidin-3-yl)butan-2-yl)amino)-1-oxohexan-2-yl)carbamate (2)

To a stirred solution of 2,2-difluoro-2-(3-fluorophenyl)-1-phenylethyl ((S)-1-oxo-1-(((S)-1-oxo-3-((S)-2-oxopyrrolidin-3-yl)propan-2-yl)amino)hexan-2-yl)carbamate (A77) (700 mg, 1.27 mmol) in DCM was added isocyanoethane (1) (1.5 ml, 2 vol) followed by acetic acid (0.2 mL, 3.56 mmol) at 0° C. and stirred at RT for 16 h. The progress of the reaction was monitored by TLC and LCMS. Reaction mixture was diluted with dichloromethane and washed with 1N HCl (2×20 mL) followed by brine (1×20 mL). Organic layer was dried over anhydrous Na₂SO₄ and evaporated under reduced pressure. Obtained material was purified by column chromatography using silica gel and 3% MeOH in DCM as eluent to afford 2,2-difluoro-2-(3-fluorophenyl)-1-phenylethyl ((2S)-1-(((2S)-4-(ethylamino)-3-acetate-4-oxo-1-((R)-2-oxopyrrolidin-3-yl)butan-2-yl)amino)-1-oxohexan-2-yl)carbamate (360 mg, 542.98 mmol) which was dissolved in THE (10 mL), water (5 mL) was added lithium hydroxide (71 mg, 1.62 mmol) at 0° C. and stirred at room temperature for 4 h. The progress of the reaction was monitored by TLC and LCMS. After completion of the reaction, reaction mixture was extracted with ethylacetate (2×100 mL), dried over anhy·sodium sulfate and concentrated under reduced pressure to afford 2,2-difluoro-2-(3-fluorophenyl)-1-phenylethyl ((2S)-1-(((2S)-4-(ethylamino)-3-hydroxy-4-oxo-1-((R)-2-oxopyrrolidin-3-yl)butan-2-yl)amino)-1-oxohexan-2-yl)carbamate (2). TLC system: 10% Methanol in dichloromethane Rf: 0.1 LCMS (ESI): m/z 621.70 [M+H]⁺

### 2,2-Difluoro-2-(3-fluorophenyl)-1-phenylethyl ((S)-1-(((S)-4-(ethylamino)-3,4-dioxo-1-((R)-2-oxopyrrolidin-3-yl)butan-2-yl)amino)-1-oxohexan-2-yl)carbamate (A173)

To a stirred solution of 2,2-difluoro-2-(3-fluorophenyl)-1-phenylethyl ((2S)-1-(((2S)-4-(ethylamino)-3-hydroxy-4-oxo-1-((R)-2-oxopyrrolidin-3-yl)butan-2-yl)amino)-1-oxohexan-2-yl)carbamate (2) (0.25 g, 0.40 mmol) in ethyl acetate (10 mL) was added Dess-Martin periodinane (381 mg, 0.80 mmol) at 0° C. and stirred at RT for 3 h. The progress of the reaction was monitored by TLC and LCMS. Reaction mixture was filter through diatomaceous earth pad and washed with ethyl acetate (20 mL) and filtrate was washed with hypo solution (3×20 mL) followed by saturated NaHCO₃ solution (3×20 mL). Organic layer was dried over anhydrous Na₂SO₄, filtered and concentrated to get which was purified by reverse phase purification and 50% ACN in 0.1% aqueous formic acid to afford 2,2-difluoro-2-(3-fluorophenyl)-1-phenylethyl ((S)-1-(((S)-4-(ethylamino)-3,4-dioxo-1-((R)-2-oxopyrrolidin-3-yl)butan-2-yl)amino)-1-oxohexan-2-yl) carbamate (A173). TLC system: 10% Methanol in dichloromethane Rf: 0.4 LCMS (ESI): m/z 619.3 [M+H]⁺

### Example 12: Synthesis of Compounds A220 and A221

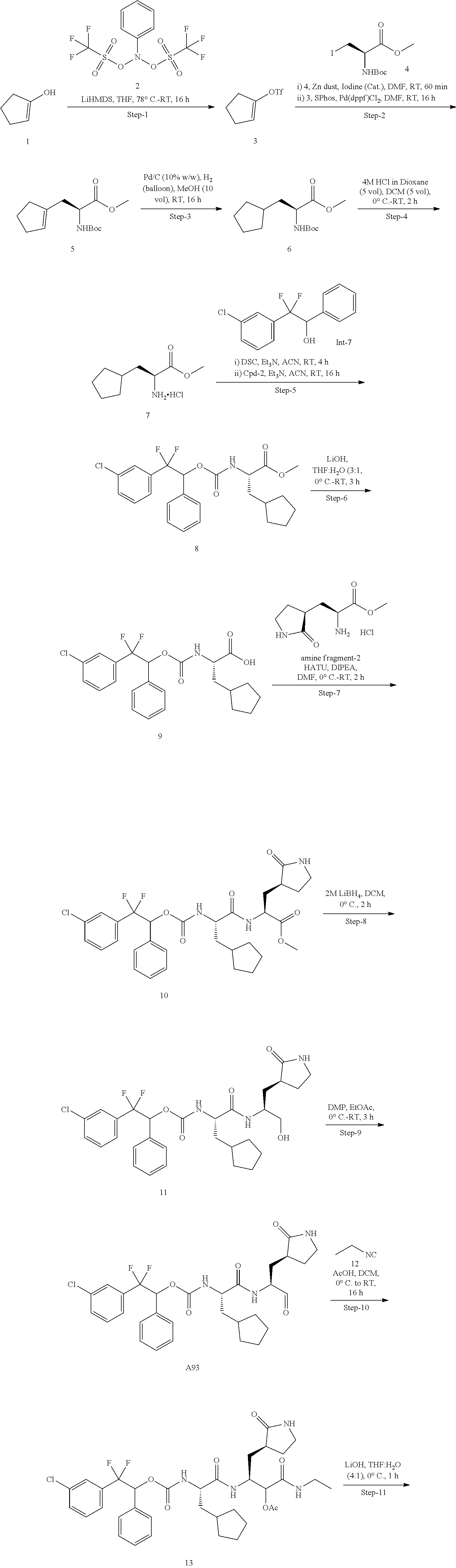

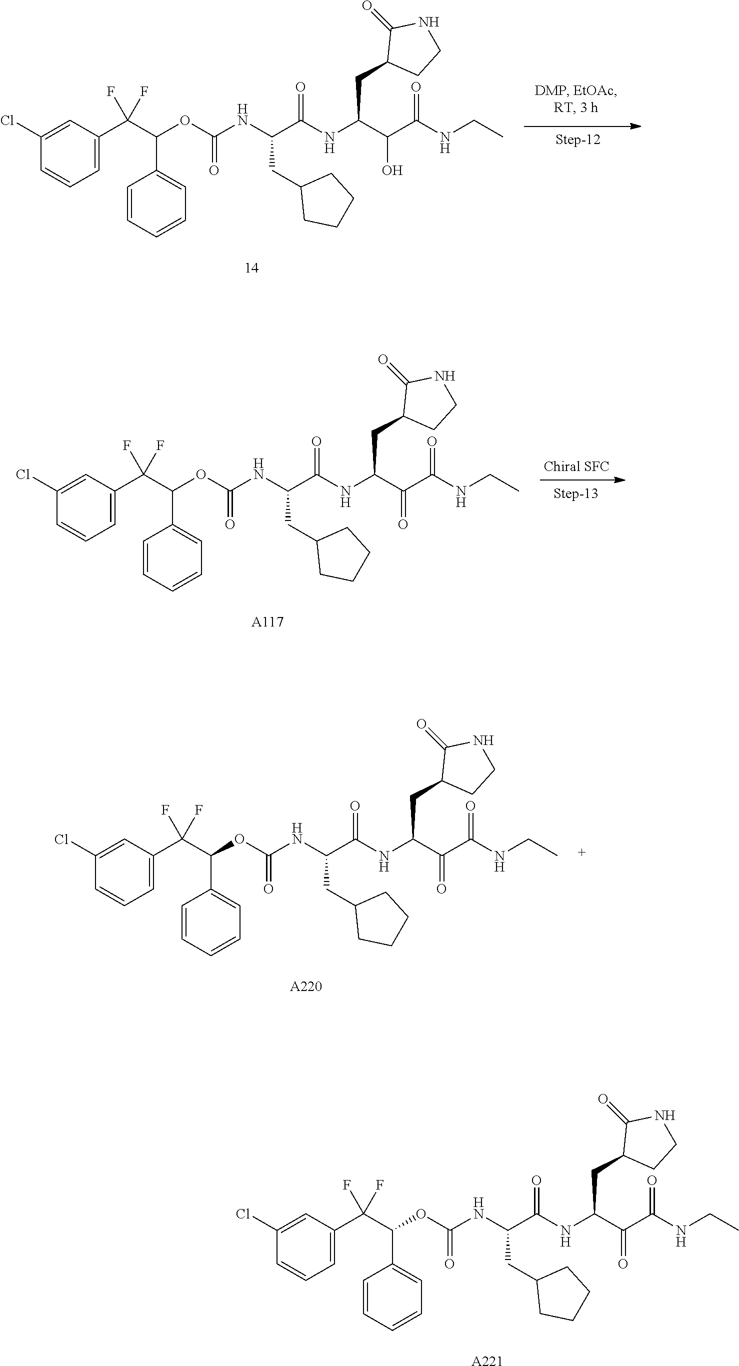

### Cyclopent-1-en-1-yl trifluoromethanesulfonate (3)

To a stirred solution of cyclopent-1-en-1-ol (1) (40 g, 476.19 mmol) in THE (200 mL) was added 1M LiHMDS in THE (153 mL, 428 mmol) dropwise at −78° C. and maintained at same for 30 min. Then added N-phenyl-O-((trifluoromethyl)sulfonyl)-N-(((trifluoromethyl) sulfonyl) oxy)hydroxylamine (152.9 g, 428.57 mmol) dissolved in THE (200 mL) drop wise at −78° C. and maintained at RT for 16 h. The progress of the reaction was monitored by TLC and LCMS. After completion of the reaction, reaction mixture was quenched with sat. Ammonium chloride and extracted with ethyl acetate (2×300 mL). Organic layer was washed with water (2×100 mL) and brine solution (50 mL), dried over anhydrous Na₂SO₄ and evaporated under reduced pressure. Obtained was purified by 100-200 silica column eluting with 2% Ethyl acetate/Pet-ether afford cyclopent-1-en-1-yl trifluoromethanesulfonate (3). TLC system: 2% Ethylaceate/Pet-ether Rf: 0.6 Methyl (S)-2-((tert-butoxycarbonyl)amino)-3-(cyclopent-1-en-1-yl)propanoate (5)

To a stirred solution of zinc dust (15.6 g, 182.37 mmol) and catalytic amount of iodine in DMF (200 mL) was added methyl (R)-2-((tert-butoxycarbonyl)amino)-3-iodopropanoate (4) (20 g, 60.79 mmol) slowly portion wise at RT and stirred for 30 min. After that added cyclopent-1-en-1-yl trifluoromethanesulfonate (3) (13.1 g, 60.79 mmol) at RT and degassed with argon for 10 min. Then added Sphos (496 mg, 1.21 mmol), Pd(dppf)Cl₂ (888 mg, 1.21 mmol) and again degassed with argon for 10 min and stirred at 50° C. for 16 h. The progress of the reaction was monitored by TLC. After completion of the reaction, reaction mixture was quenched with ice water (500 mL) and extracted with ethyl acetate (2×200 mL). Combined organic layer was washed with brine solution (100 mL), dried over anhydrous sodium sulfate and evaporated under reduced pressure. Obtained was purified by silica (100-200 mesh) column and eluting with 5% Ethyl acetate/Pet-ether afforded methyl (S)-2-((tert-butoxycarbonyl) amino)-3-(cyclopent-1-en-1-yl) propanoate (5). TLC system: 2% Ethylaceate/Pet-ether Rf: 0.4 Methyl (S)-2-((tert-butoxycarbonyl)amino)-3-cyclopentylpropanoate (6)

To a stirred solution of methyl (S)-2-((tert-butoxycarbonyl)amino)-3-(cyclopent-1-en-1-yl)propanoate (5) (10 g, 37.17 mmol) in methanol (100 mL) was added 10% Pd—C (20% w/w) at RT and stirred under nitrogen balloon pressure for 16 h. The progress of the reaction was monitored by TLC. Reaction mixture was filtered through diatomaceous earth bed, washed the bed with methanol (50 mL) and filtrate was evaporated under reduced pressure to afford methyl (S)-2-((tert-butoxycarbonyl)amino)-3-cyclopentylpropanoate (6). TLC system: 4% Ethylaceate/Pet-ether Rf: 0.5 Methyl (S)-2-amino-3-cyclopentylpropanoate hydrochloride (7):

To a stirred solution of methyl (S)-2-((tert-butoxycarbonyl)amino)-3-cyclopentylpropanoate (6) (19.1 g, 70.47 mmol) in DCM (40 mL) was added 4M 1,4-Dioxane·HCl (76 mL, 4 vol) at 0° C. and stirred at Room temperature for 2 h.

The progress of the reaction was monitored by TLC. Reaction mixture was evaporated under reduced pressure to afford material, which was triturated with diethyl ether (2×50 mL) to afford methyl (S)-2-amino-3-cyclopentylpropanoate hydrochloride (7). TLC system: 10% MeOH in DCM Rf: 0.2

### Methyl (2S)-2-(((2-(3-chlorophenyl)-2,2-difluoro-1-phenylethoxy)carbonyl)amino)-3-cyclopentylpropanoate (8)

To a stirred solution of 2-(3-chlorophenyl)-2,2-difluoro-1-phenylethan-1-ol (Int-7) (22 g, 82.089 mmol) in Acetonitrile 100 mL was added DSC (42 g, 164.17 mmol), followed by triethyl amine (28.5 mL, 205.22 mmol) at 0° C. and stirred the reaction mixture at room temperature for 2 h.

In another RB flask, methyl (S)-2-amino-3-cyclopentylpropanoate hydrochloride (7) (13 g, 62.8 mmol) was taken in Acetonitrile (120 mL), and treated with triethylamine (21.8 mL, 75.36 mmol). Then added above prepared reaction mass drop-wise and the reaction mixture was stirred at room temperature for 16 h. Reaction mixture was quenched with ice water (150 mL) and extracted with ethyl acetate (2×100 mL), combined organic layers were washed with brine solution (100 mL), dried over sodium sulfate and evaporated under reduced pressure to get the compound.

The material was purified by normal phase chromatography to afford methyl (2S)-2-(((2-(3-chlorophenyl)-2,2-difluoro-1-phenylethoxy)carbonyl)amino)-3-cyclopentylpropanoate (8). TLC system: 10% Ethyl acetate/Pet ether R_{f}: 0.4 LCMS (ESI): m/z 466.27 [M+H]⁺

### (2S)-2-(((2-(3-Chlorophenyl)-2,2-difluoro-1-phenylethoxy)carbonyl)amino)-3-cyclopentylpropanoic acid (9)

To a stirred solution of methyl (2S)-2-(((2-(3-chlorophenyl)-2,2-difluoro-1-phenylethoxy)carbonyl)amino)-3-cyclopentylpropanoate (8) (23 g, 49.67 mmol) in THE (150 mL), water (10 mL) was added lithium hydroxide (5.2 g, 124.17 mmol) at RT and stirred at room temperature for 2 h. The progress of the reaction was monitored by TLC and LCMS. Excess of THE was distilled under reduced pressure, compound acidified with aq. 1N HCl solution up to pH˜2 and extracted with DCM (2×500 mL), combined organic layers were washed with water (200 mL) brine solution (150 mL), dried over sodium sulfate, concentrated under reduced pressure to afford (2S)-2-(((2-(3-chlorophenyl)-2,2-difluoro-1-phenylethoxy)carbonyl)amino)-3-cyclopentylpropanoic acid (9). TLC system: 10% Methanol in dichloromethane Rf 0.2 LCMS (ESI): m/z 452.25 [M+H]⁺

### Methyl (2S)-2-((2S)-2-(((2-(3-chlorophenyl)-2,2-difluoro-1-phenylethoxy)carbonyl)amino)-3-cyclopentylpropanamido)-3-((S)-2-oxopyrrolidin-3-yl)propanoate (10)

To a stirred solution of (2S)-2-(((2-(3-chlorophenyl)-2,2-difluoro-1-phenylethoxy) carbonyl)amino)-3-cyclopentylpropanoic acid (9) (17 g, 37.69 mmol) in DMF (170 mL) was added HATU (21.4 g, 56.53 mmol), DIPEA (20 mL, 113.07 mmol) and methyl (S)-2-amino-3-((S)-2-oxopyrrolidin-3-yl)propanoate hydrochloride (amine fragment-2) (10 g, 45.23 mmol) at 0° C. simultaneously and stirred at room temperature for 16 h. Reaction mixture was diluted with ice water (400 mL), extracted with ethyl acetate (2×200 mL). Organic layer was separated, dried over anhydrous sodium sulphate and evaporated under reduced pressure. The residue was purified by normal phase combiflash column to afford methyl (2S)-2-((2S)-2-(((2-(3-chlorophenyl)-2,2-difluoro-1-phenylethoxy) carbonyl)amino)-3-cyclopentylpropanamido)-3-((S)-2-oxopyrrolidin-3-yl) propanoate (10). TLC system: 5% MeOH/DCM Rf: 0.45 LCMS (ESI): m/z=620.34 [M+H]⁺

### 2-(3-Chlorophenyl)-2,2-difluoro-1-phenylethyl ((S)-3-cyclopentyl-1-(((S)-1-hydroxy-3-((S)-2-oxopyrrolidin-3-yl)propan-2-yl)amino)-1-oxopropan-2-yl)carbamate (11)

To a stirred solution of methyl (2S)-2-((2S)-2-(((2-(3-chlorophenyl)-2,2-difluoro-1-phenylethoxy) carbonyl) amino)-3-cyclopentylpropanamido)-3-((S)-2-oxopyrrolidin-3-yl) propanoate (10) (1.5 g, 2.42 mmol) in dichloromethane (20 mL) was added 2M LiBH₄ in THE (2.4 mL, 4.84 mmol) slowly drop wise at 0° C. and stirred for 2 h at 0° C. The progress of the reaction was monitored by TLC. Reaction mixture was quenched with saturated NH₄Cl solution and extracted with dichloromethane (2×100 mL), dried over anhydrous sodium sulfate and concentrated under reduced pressure to afford 2-(3-chlorophenyl)-2,2-difluoro-1-phenylethyl ((S)-3-cyclopentyl-1-(((S)-1-hydroxy-3-((S)-2-oxopyrrolidin-3-yl)propan-2-yl)amino)-1-oxopropan-2-yl)carbamate (11). TLC system: 10% Methanol in dichloromethane Rf: 0.55 LCMS (ESI): m/z 592.27 [M+H]⁺

### 2-(3-chlorophenyl)-2,2-difluoro-1-phenylethyl ((S)-3-cyclopentyl-1-oxo-1-(((S)-1-oxo-3-((S)-2-oxopyrrolidin-3-yl)propan-2-yl)amino)propan-2-yl)carbamate (A93)

To a stirred solution of 2-(3-chlorophenyl)-2,2-difluoro-1-phenylethyl ((S)-3-cyclopentyl-1-(((S)-1-hydroxy-3-((S)-2-oxopyrrolidin-3-yl)propan-2-yl)amino)-1-oxopropan-2-yl)carbamate (11) (2.1 g, 3.55 mmol) in ethyl acetate (25 mL) was added Dess-Martin periodinane (2.2 g, 5.32 mmol) at 0° C. and stirred at RT for 3 h. The progress of the reaction was monitored by TLC and LCMS. Reaction mixture was diluted with ethyl acetate (20 mL) and washed with sat. NaHCO₃ solution (3×30 mL) followed by sat. Hypo solution (3×30 mL). Organic layer was dried over anhydrous Na₂SO₄, filtered and concentrated under reduced pressure to afford 2-(3-chlorophenyl)-2,2-difluoro-1-phenylethyl ((S)-3-cyclopentyl-1-oxo-1-(((S)-1-oxo-3-((S)-2-oxopyrrolidin-3-yl) propan-2-yl)amino)propan-2-yl)carbamate (A93). TLC system: 10% Methanol in DCM R_{f}: 0.55 LCMS (ESI): m/z 590.3 [M+H]⁺

### 2-(3-Chlorophenyl)-2,2-difluoro-1-phenylethyl ((2S)-3-cyclopentyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (13)

To a stirred solution of 2-(3-chlorophenyl)-2,2-difluoro-1-phenylethyl ((S)-3-cyclopentyl-1-oxo-1-(((S)-1-oxo-3-((S)-2-oxopyrrolidin-3-yl)propan-2-yl)amino)propan-2-yl)carbamate (A93) (4 g, 6.78 mmol) was dissolved in DCM (40 mL), then added ethyl isocyanide solution in DCM (10 mL) (14.24 mmol) at 0° C. and stirred up to RT for 6 h. The progress of the reaction was monitored by TLC and LCMS. Reaction mixture was diluted with dichloromethane and washed with 1N HCl (2×15 mL) followed by water (2×20 mL) and brine (20 mL). Organic layer was dried over anhydrous Na₂SO₄ and evaporated under reduced pressure to afford material which was purified by Reverse phase column (C18) and eluted with 1% ammonium bicarbonate/acetonitrile to afford (6S,9S)-1-(3-chlorophenyl)-6-(cyclopentylmethyl)-1,1-difluoro-4,7,11-trioxo-9-(((S)-2-oxopyrrolidin-3-yl) methyl)-2-phenyl-3-oxa-5,8,12-triazatetradecan-10-yl acetate (13). TLC system: 5% MeOH in DCM Rf: 0.4 LCMS (ESI): m/z 705.33 [M+H]⁺

### 2-(3-chlorophenyl)-2,2-difluoro-1-phenylethyl ((2S)-3-cyclopentyl-1-(((2S)-4-(ethylamino)-3-hydroxy-4-oxo-1-((S)-2-oxopyrrolidin-3-yl)butan-2-yl)amino)-1-oxopropan-2-yl)carbamate (14)

To a stirred solution of (6S,9S)-1-(3-chlorophenyl)-6-(cyclopentylmethyl)-1,1-difluoro-4,7,11-trioxo-9-(((S)-2-oxopyrrolidin-3-yl)methyl)-2-phenyl-3-oxa-5,8,12-triazatetradecan-10-yl acetate (13) (4 g, 14.9 mmol) in THF: H₂O (30 mL) (4:1) was added LiOH·H₂O (939 mg, 22.35 mmol) at 0° C. and stirred at same for 2 h. The progress of the reaction was monitored by TLC and LCMS. Reaction mixture was quenched with water and extracted with ethyl acetate (2×100 mL) and washed with brine solution (20 mL). Organic layer was dried over anhydrous Na₂SO₄, filtered and concentrated under reduced pressure to afford 2-(3-chlorophenyl)-2,2-difluoro-1-phenylethyl ((2S)-3-cyclopentyl-1-(((2S)-4-(ethylamino)-3-hydroxy-4-oxo-1-((S)-2-oxopyrrolidin-3-yl) butan-2-yl)amino)-1-oxopropan-2-yl)carbamate (14). LCMS (ESI): m/z 663.50 [M+H]⁺

### 2-(3-chlorophenyl)-2,2-difluoro-1-phenylethyl ((S)-3-cyclopentyl-1-(((S)-4-(ethylamino)-3,4-dioxo-1-((S)-2-oxopyrrolidin-3-yl)butan-2-yl)amino)-1-oxopropan-2-yl)carbamate (A117)

To a stirred solution of 2-(3-chlorophenyl)-2,2-difluoro-1-phenylethyl ((2S)-3-cyclopentyl-1-(((2S)-4-(ethylamino)-3-hydroxy-4-oxo-1-((S)-2-oxopyrrolidin-3-yl)butan-2-yl)amino)-1-oxopropan-2-yl)carbamate (14) (5 g, 7.55 mmol) in ethyl acetate (50 mL) was added Dess-Martin periodinane (6.4 g, 15.1 mmol) at 0° C. and stirred at RT for 6 h. The progress of the reaction was monitored by TLC and LCMS. Reaction mixture was filtered through diatomaceous earth pad washed with ethyl acetate (50 mL) and filtrate was washed with hypo solution (3×50 mL) followed by saturated NaHCO₃ solution (3×50 mL) and brine solution (20 mL). Organic layer was dried over anhydrous Na₂SO₄, filtered and concentrated under reduced pressure. Obtained material was purified by trituration with n-pentane/DEE to afford 2-(3-chlorophenyl)-2,2-difluoro-1-phenylethyl ((S)-3-cyclopentyl-1-(((S)-4-(ethylamino)-3,4-dioxo-1-((S)-2-oxopyrrolidin-3-yl)butan-2-yl)amino)-1-oxopropan-2-yl)carbamate (A117). TLC system: 10% MeOH in DCM Rf: 0.6 LCMS (ESI): m/z 661.29 [M+H]⁺

### (S)-2-(3-chlorophenyl)-2,2-difluoro-1-phenylethyl ((S)-3-cyclopentyl-1-(((S)-4-(ethylamino)-3,4-dioxo-1-((S)-2-oxopyrrolidin-3-yl)butan-2-yl)amino)-1-oxopropan-2-yl)carbamate (A220) and (R)-2-(3-chlorophenyl)-2,2-difluoro-1-phenylethyl ((S)-3-cyclopentyl-1-(((S)-4-(ethylamino)-3,4-dioxo-1-((S)-2-oxopyrrolidin-3-yl)butan-2-yl)amino)-1-oxopropan-2-yl)carbamate (A221)

2-(3-chlorophenyl)-2,2-difluoro-1-phenylethyl ((S)-3-cyclopentyl-1-(((S)-4-(ethylamino)-3,4-dioxo-1-((S)-2-oxopyrrolidin-3-yl)butan-2-yl)amino)-1-oxopropan-2-yl)carbamate (A117) (4 g, 60.4 mmol) was purified by SFC chiral purification to afford (S)-2-(3-chlorophenyl)-2,2-difluoro-1-phenylethyl ((S)-3-cyclopentyl-1-(((S)-4-(ethylamino)-3,4-dioxo-1-((S)-2-oxopyrrolidin-3-yl)butan-2-yl)amino)-1-oxopropan-2-yl)carbamate (A220) and (R)-2-(3-chlorophenyl)-2,2-difluoro-1-phenylethyl ((S)-3-cyclopentyl-1-(((S)-4-(ethylamino)-3,4-dioxo-1-((S)-2-oxopyrrolidin-3-yl) butan-2-yl)amino)-1-oxopropan-2-yl)carbamate (A221).

### Example 13: Synthesis of Compound A223

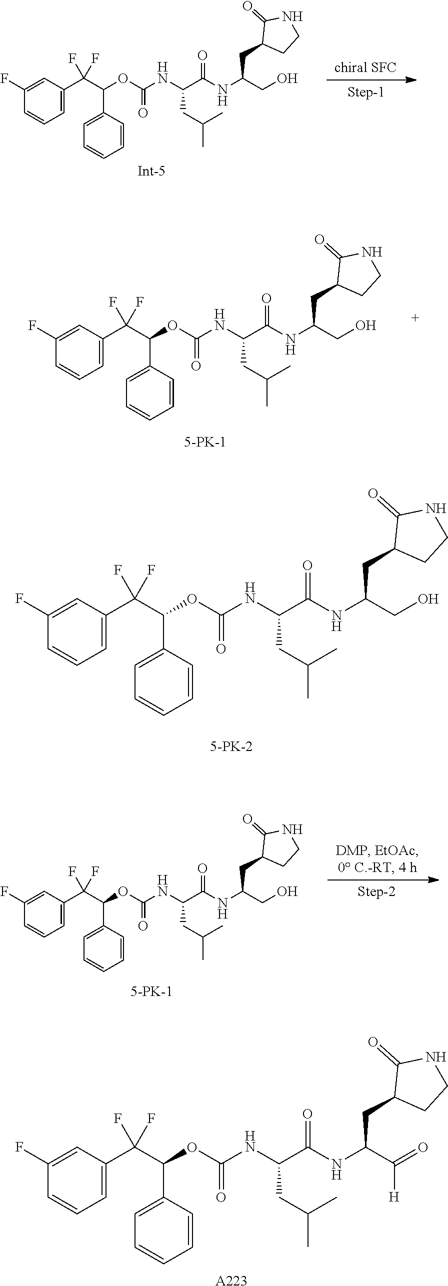 [SMILES: CC(C)C[C@H](NC(=O)OC(c1ccccc1)C(F)(F)c1cccc(F)c1)C(=O)N[C@H](CO)C[C@@H]1CCNC1=O ; CC(C)C[C@H](NC(=O)O[C@@H](c1ccccc1)C(F)(F)c1cccc(F)c1)C(=O)N[C@H](CO)C[C@@H]1CCNC1=O ; CC(C)C[C@H](NC(=O)O[C@H](c1ccccc1)C(F)(F)c1cccc(F)c1)C(=O)N[C@H](CO)C[C@@H]1CCNC1=O ; CC(C)C[C@H](NC(=O)O[C@@H](c1ccccc1)C(F)(F)c1cccc(F)c1)C(=O)N[C@H](CO)C[C@@H]1CCNC1=O ; CC(C)C[C@H](NC(=O)O[C@@H](c1ccccc1)C(F)(F)c1cccc(F)c1)C(=O)N[C@H](C=O)C[C@@H]1CCNC1=O ; "A223"]

### (S)-2,2-difluoro-2-(3-fluorophenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (5-PK-1) and (R)-2,2-difluoro-2-(3-fluorophenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (5-PK-2)

2,2-difluoro-2-(3-fluorophenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (Int-5) (2.3 g) was separated by chiral SFC purification to afford (S)-2,2-difluoro-2-(3-fluorophenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (5-PK-1) (800 mg) and (R)-2,2-difluoro-2-(3-fluorophenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (5-PK-2). LCMS (ESI): m/z 550.3 [M+H]⁺

### (S)-2,2-difluoro-2-(3-fluorophenyl)-1-phenylethyl ((S)-4-methyl-1-oxo-1-(((S)-1-oxo-3-((S)-2-oxopyrrolidin-3-yl)propan-2-yl)amino)pentan-2-yl)carbamate (A223)

To a stirred solution of (S)-2,2-difluoro-2-(3-fluorophenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (5-PK-1) (260 mg, 0.47 mmol) in EtOAc (10 mL) was added Dess-Martin periodinane (602 mg, 1.42 mmol) at 0° C. and stirred at RT for 2 h. After completion of the reaction (monitored by TLC), reaction mixture was diluted with EtOAc (20 mL) and filtered through diatomaceous earth pad. Obtained filtrate was washed with sat. Hypo solution (3×30 mL) followed by sat. NaHCO₃ solution (3×20 mL). Organic layer was dried over anhydrous Na₂SO₄, filtered and concentrated under reduced pressure. Obtained compound was purified reverse phase combiflash column (C18) and 45% of ACN in 1% aq. NH₄CO₃ solution used as eluent to afford (S)-2,2-difluoro-2-(3-fluorophenyl)-1-phenylethyl ((S)-4-methyl-1-oxo-1-(((S)-1-oxo-3-((S)-2-oxo pyrrolidin-3-yl)propan-2-yl)amino)pentan-2-yl)carbamate A223. TLC system: 5% Methanol in Dichloromethane LCMS (ESI): m/z=548.3 (M+H)⁺ Rf: 0.5

### Example 14: Synthesis of Compound A224

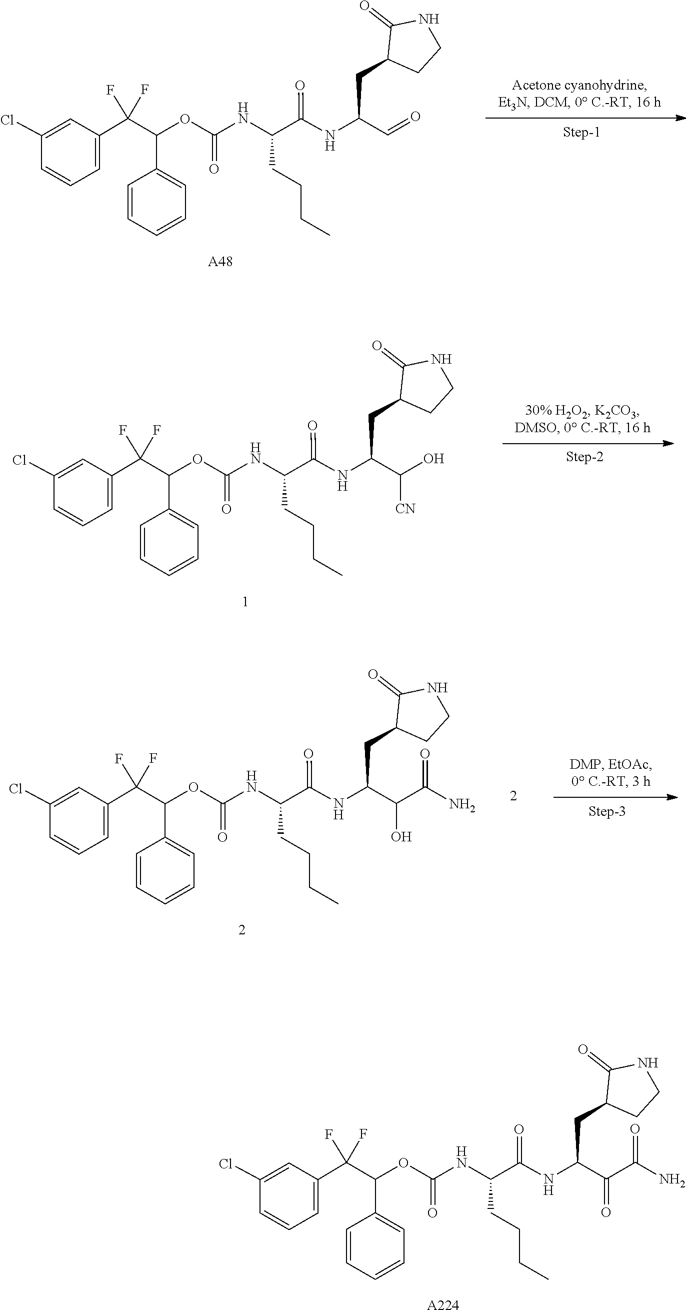

### 2-(3-Chlorophenyl)-2,2-difluoro-1-phenylethyl ((2S)-1-(((2S)-1-cyano-1-hydroxy-3-((S)-2-oxopyrrolidin-3-yl) propan-2-yl) amino)-1-oxohexan-2-yl) carbamate (1)

To a stirred solution of 2-(3-chlorophenyl)-2,2-difluoro-1-phenylethyl ((S)-1-oxo-1-(((S)-1-oxo-3-((S)-2-oxopyrrolidin-3-yl)propan-2-yl)amino)hexan-2-yl)carbamate (A48) (600 mg, 1.063 mmol) in DCM (10 mL) was added acetonecyanohydrine (0.6 mL, 1 vol), Et₃N (0.6 mL, 1 vol) at 0° C. and stirred at RT for 16 h. The progress of the reaction was monitored by TLC. The reaction mixture was diluted with DCM (15 mL) and washed with water (2×15 mL), dried over sodium sulfate and evaporated under reduced pressure to get the product. This material was triturated with n-pentane (25 mL) to afford 2-(3-chlorophenyl)-2,2-difluoro-1-phenylethyl ((2S)-1-(((2S)-1-cyano-1-hydroxy-3-((S)-2-oxopyrrolidin-3-yl)propan-2-yl)amino)-1-oxohexan-2-yl)carbamate (1). TLC system: 10% Methanol in dichloromethane Rf: 0.5 LCMS (ESI): m/z 591.35 [M+H]⁺

### 2-(3-Chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-1-oxohexan-2-yl) carbamate (2)

To a stirred solution of 2-(3-chlorophenyl)-2,2-difluoro-1-phenylethyl ((2S)-1-(((2S)-1-cyano-1-hydroxy-3-((S)-2-oxopyrrolidin-3-yl) propan-2-yl) amino)-1-oxohexan-2-yl) carbamate (1) (450 mg, 0.762 mmol) in DMSO (5 mL) was added potassium carbonate (210 mg, 1.525 mmol) followed by 30% H₂O₂ (0.8 mL, 2 vol) at 0° C. and stirred at RT for 16 h. The progress of the reaction was monitored by TLC. The reaction mixture was quenched with Sat. ammonium chloride solution (20 mL) and extracted with ethyl acetate (2×20 mL). The combined organic layer was washed with water (2×15 mL) followed by brine (1×15 mL), dried over sodium sulfate and evaporated under reduced pressure to get the material, which was triturated with n-pentane (15 mL) to afford 2-(3-chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-1-oxohexan-2-yl) carbamate (2). TLC system: 10% Methanol in dichloromethane Rf: 0.3 LCMS (ESI): m/z 609.39 [M+H]⁺

### 2-(3-Chlorophenyl)-2,2-difluoro-1-phenylethyl ((S)-1-(((S)-4-amino-3,4-dioxo-1-((S)-2-oxopyrrolidin-3-yl)butan-2-yl)amino)-1-oxohexan-2-yl)carbamate (A224)

To a stirred solution of 2-(3-chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-1-oxohexan-2-yl) carbamate (2) (300 mg, 0.492 mmol) in ethyl acetate (10 mL) was added Dess-Martin periodinane (417 mg, 0.985 mmol) at 0° C. and stirred at RT for 3 h. The progress of the reaction was monitored by TLC and LC-MS. The reaction mixture was filtered through diatomaceous earth bed and washed with ethyl acetate (20 mL). Filtrate was washed with sat. Hypo solution (3×20 mL), sat. NaHCO₃ solution (3×20 mL) followed by brine (1×15 mL). Organic layer was dried over anhydrous Na₂SO₄, filtered and concentrated to get the material, which was purified by reverse phase chromatography by using 0.1% ammonium carbonate in water/acetonitrile as buffer to afford 2-(3-chlorophenyl)-2,2-difluoro-1-phenylethyl ((S)-1-(((S)-4-amino-3,4-dioxo-1-((S)-2-oxo pyrolidin-3-yl) butan-2-yl) amino)-1-oxohexan-2-yl) carbamate (A224). TLC system: 10% Methanol in dichloromethane Rf: 0.4 LCMS (ESI): m/z 607.2 [M+H]⁺

### Example 15: Synthesis of Compounds A222 and A226

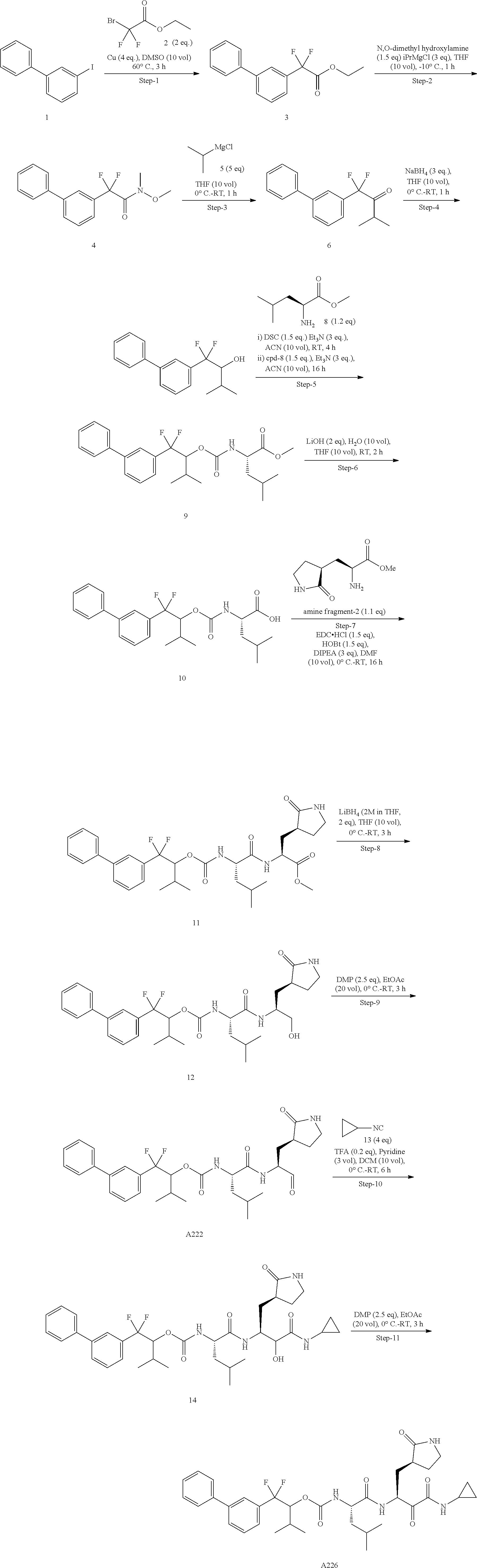

### Ethyl 2-([1,1′-biphenyl]-3-yl)-2,2-difluoroacetate (3)

Ethyl 2-bromo-2,2-difluoroacetate (2) (28.97 g, 142.857 mmol) was added to a suspension of copper powder (18.52 g, 285.612 mmol) in DMSO (100 mL) under N₂. The suspension was stirred for 1 h at room temperature, then was added 3-iodo-1,1′-biphenyl (20 g, 71.4030 mmol) and the stirred at 60° C. for 3 h. The progress of the reaction was monitored by TLC. The reaction mixture was quenched with Aq. NH₄Cl solution (200 mL), and extracted with diethyl ether (2×150 mL). The combined organic layer was washed with water (2×200 mL), brine (1×100 mL), dried over sodium sulfate and evaporated to get the material, which was purified by normal phase chromatography to afford ethyl 2-([1,1′-biphenyl]-3-yl)-2,2-difluoroacetate (3). TLC system: 10% Ethyl acetate/Pet ether Rf: 0.4 LCMS (ESI): m/z 257.06 [M−F]⁺

### 2-([1,1′-biphenyl]-3-yl)-2,2-difluoro-N-methoxy-N-methylacetamide (4)

To a stirred solution of ethyl 2-([1,1′-biphenyl]-3-yl)-2,2-difluoroacetate (3) (12 g, 43.478 mmol) in THE (100 mL) was added N,O-dimethyl hydroxylamine. hydrochloride (6.32 g, 65.217 mmol), and the reaction was cooled to −10° C., then isopropyl magnesium chloride 1.0 M in THE (130.434 mL, 130.434 mmol) was added slowly and stirred for 1 h at same temperature. The progress of the reaction was monitored by TLC. The reaction mixture was quenched with Sat. ammonium chloride solution (100 mL) and extracted with ethyl acetate (2×100 mL). The combined organic layer was washed with water (1×100 mL), followed by brine (1×100 mL), dried over sodium sulfate and evaporated under reduced pressure to afford 2-([1,1′-biphenyl]-3-yl)-2,2-difluoro-N-methoxy-N-methylacetamide (4). TLC system: 20% Ethyl acetate in pet ether Rf: 0.5 LCMS (ESI): m/z 292.10 [M+H]⁺

### 1-([1,1′-Biphenyl]-3-yl)-1,1-difluoro-3-methylbutan-2-one (6)

To a stirred solution of 2-([1,1′-biphenyl]-3-yl)-2,2-difluoro-N-methoxy-N-methyl acetamide (4) (10 g, 34.364 mmol) in THE (100 mL) wad added Isopropyl magnesium chloride (86 mL, 171.821 mmol) at −10° C. and stirred at RT for 2 h. The reaction mixture was quenched with sat. Ammonium chloride solution (100 mL) and extracted with ethyl acetate (2×100 mL). The combined organic layer was washed with water (50 mL), brine (150 mL), dried over sodium sulfate and evaporated under reduced pressure to afford 2-([1,1′-biphenyl]-3-yl)-2,2-difluoro-1-phenylethan-1-one (6) that was directly used in the next step. TLC system: 10% Ethyl acetate in hexane Rf: 0.4 LCMS (ESI): m/z 255.06 [M−F]⁺

### 1-([1,1′-Biphenyl]-3-yl)-1,1-difluoro-3-methylbutan-2-ol (7)

To a stirred solution of 2-([1,1′-biphenyl]-3-yl)-2,2-difluoro-1-phenylethan-1-one (6) (7.4 g, 27.007 mmol) in THE (130 mL) wad added sodium borohydride (3.062 g, 81.021 mmol) at 0° C. and stirred at RT for 2 h. Reaction progress was monitored by TLC. The reaction mixture was quenched with ammonium chloride (100 mL) and extracted with ethyl acetate (2×100 mL). The combined organic layer was washed with water (150 mL), brine (150 mL), and dried over sodium sulfate, evaporated under reduced pressure to afford product, this product was purified using combi flash (normal phase) by eluting with 15% ethyl acetate in pet ether to afford 1-([1,1′-biphenyl]-3-yl)-1,1-difluoro-3-methylbutan-2-ol. TLC system: 30% Ethyl acetate in hexane R_{f}: 0.5 LCMS (ESI): m/z 257.09 [M−F]⁺

### methyl (((1-([1,1′-biphenyl]-3-yl)-1,1-difluoro-3-methylbutan-2-yl)oxy)carbonyl)-L-leucinate (9)

To a stirred solution of 1-([1,1′-biphenyl]-3-yl)-1,1-difluoro-3-methylbutan-2-ol (7) (3.2 g, 11.594 mmol) in ACN (30 mL) was added N,N′disuccinimidyl carbonate (7.42 g, 28.985 mmol), followed by Et₃N (9.6 mL, 3 vol) at 0° C. and stirred at room temperature for 4 h. The progress of the reaction was monitored by TLC. The reaction mass was used directly in the subsequent reaction.

In another RB flask, methyl L-leucinate hydrochloride (8) (5.24 g, 28.985 mmol) was taken in ACN (20 mL), and treated with Et₃N (9.6 mL, 3 vol). The reaction mixture was stirred for 5 min, then the above prepared reaction mass was added drop-wise and the reaction mixture stirred at room temperature for 16 h. After completion of the reaction by TLC and LC-MS, the reaction mixture was quenched with ice water (100 mL) and extracted with ethyl acetate (2×100 mL). The combined organic layer was washed with brine (100 mL), dried over sodium sulfate and evaporated under reduced pressure to get the material, which was purified by combi flash (normal phase) by eluting with 5% ethyl acetate in pet ether to afford methyl (((1-([1,1′-biphenyl]-3-yl)-1,1-difluoro-3-methylbutan-2-yl)oxy)carbonyl)-L-leucinate (9). TLC system: 30% Ethyl acetate/Pet ether R_{f}: 0.4 LCMS (ESI): m/z 428.35 [M−F]⁺

### (((1-([1,1′-Biphenyl]-3-yl)-1,1-difluoro-3-methylbutan-2-yl)oxy)carbonyl)-L-leucine (10)

To a stirred solution of methyl (((1-([1,1′-biphenyl]-3-yl)-1,1-difluoro-3-methylbutan-2-yl)oxy)carbonyl)-L-leucinate (9) (1.2 g, 2.684 mmol) in THE (12 mL), water (12 mL) was added lithium hydroxide (128.5 mg, 5.369 mmol) at RT and stirred at room temperature for 2 h. The progress of the reaction was monitored by TLC and LC-MS. Excess of THE was distilled under reduced pressure, compound was acidified with aq. 1N HCl solution up to pH˜2 and extracted with ethyl acetate (2×50 mL). The combined organic layer was washed with water (50 mL) brine (50 mL), dried over sodium sulfate and concentrated under reduced pressure to afford ((2-([1,1′-biphenyl]-3-yl)-2,2-difluoro-1-phenylethoxy)carbonyl)-L-leucine (10). TLC system: 10% MeOH/DCM Rf: 0.5 LCMS (ESI): m/z 456.31 [M+Na]⁺

### Methyl (2S)-2-((2S)-2-((((1-([1,1′-biphenyl]-3-yl)-1,1-difluoro-3-methylbutan-2-yl)oxy)carbonyl)amino)-4-methylpentanamido)-3-((S)-2-oxopyrrolidin-3-yl)propanoate (11)

To a stirred solution of ((2-([1,1′-biphenyl]-3-yl)-2,2-difluoro-1-phenylethoxy)carbonyl)-L-leucine (10) (800 mg, 1.845 mmol) in DMF (20 mL) was added EDC·HCl (528.7 mg, 2.768 mmol), HOBt (373.7 mg, 2.768 mmol), DIPEA (1.02 mL, 5.536 mmol) and methyl (S)-2-amino-3-((S)-2-oxopyrrolidin-3-yl)propanoate hydrochloride (amine fragment-2) (411.9 mg, 2.214 mmol) at 0° C. The reaction mixture was allowed to stir at room temperature for 16 h. Water was (100 mL) added to the reaction mixture and extracted with ethyl acetate (2×50 mL). The combined organic layer was dried over sodium sulphate and evaporated under reduced pressure to afford compound. The material was purified by normal phase chromatography to afford methyl (2S)-2-((2S)-2-((((1-([1,1′-biphenyl]-3-yl)-1,1-difluoro-3-methylbutan-2-yl)oxy)carbonyl)amino)-4-methylpentanamido)-3-((S)-2-oxopyrrolidin-3-yl)propanoate (11). TLC system:10% Methanol in dichloromethane Rf: 0.4 LCMS (ESI): m/z=602.31 [M+H]⁺

### 1-([1,1′-Biphenyl]-3-yl)-1,1-difluoro-3-methylbutan-2-yl ((S)-1-(((S)-1-hydroxy-3-((S)-2-oxopyrrolidin-3-yl)propan-2-yl)amino)-4-methyl-1-oxopentan-2-yl)carbamate (12)

To a stirred solution of methyl (2S)-2-((2S)-2-((((1-([1,1′-biphenyl]-3-yl)-1,1-difluoro-3-methylbutan-2-yl)oxy)carbonyl)amino)-4-methylpentanamido)-3-((S)-2-oxopyrrolidin-3-yl)propanoate (11) (500 mg, 0.830 mmol) in THE (10 mL) was added 2M LiBH₄ in THE (0.83 mL, 1.661 mmol at 0° C. and stirred for 3 h at rt. The progress of the reaction was monitored by TLC and LC-MS. The reaction mixture was quenched with saturated NH₄Cl solution (10 mL) and extracted with ethyl acetate (2×25 mL), dried over sodium sulfate, concentrated under reduced pressure to afford 1-([1,1′-biphenyl]-3-yl)-1,1-difluoro-3-methylbutan-2-yl ((S)-1-(((S)-1-hydroxy-3-((S)-2-oxopyrrolidin-3-yl)propan-2-yl)amino)-4-methyl-1-oxopentan-2-yl)carbamate (12). TLC system: 10% Methanol in dichloromethane Rf: 0.3 LCMS (ESI): m/z 574.48 [M+H]⁺

### 1-([1,1′-Biphenyl]-3-yl)-1,1-difluoro-3-methylbutan-2-yl ((S)-4-methyl-1-oxo-1-(((S)-1-oxo-3-((S)-2-oxopyrrolidin-3-yl)propan-2-yl)amino)pentan-2-yl)carbamate (A222)

To a stirred solution 1-([1,1′-biphenyl]-3-yl)-1,1-difluoro-3-methylbutan-2-yl ((S)-1-(((S)-1-hydroxy-3-((S)-2-oxopyrrolidin-3-yl)propan-2-yl)amino)-4-methyl-1-oxopentan-2-yl)carbamate (12) (100 mg, 0.174 mmol) in ethyl acetate (3 mL) was added Dess-Martin periodinane (110.8 mg, 0.2614 mmol) at 0° C. and stirred at RT for 3 h. The progress of the reaction was monitored by TLC and LC-MS. The suspension was filtered through a pad of diatomaceous earth and washed with ethyl acetate and the filtrate was washed with sat. NaHCO₃ solution (3×30 mL) followed by sat. Hypo solution (3×30 mL). The organic layer was dried over anhydrous Na₂SO₄, filtered and concentrated to get material, which was triturated with n-pentane/diethyl ether to afford 1-([1,1′-biphenyl]-3-yl)-1,1-difluoro-3-methylbutan-2-yl ((S)-4-methyl-1-oxo-1-(((S)-1-oxo-3-((S)-2-oxopyrrolidin-3-yl)propan-2-yl)amino)pentan-2-yl)carbamate (A222). TLC system: 10% Methanol in DCM R_{f}: 0.4 LCMS (ESI): m/z 572.3 [M+H]⁺

### 1-([1,1′-Biphenyl]-3-yl)-1,1-difluoro-3-methylbutan-2-yl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (14)

To a stirred solution of 1-([1,1′-biphenyl]-3-yl)-1,1-difluoro-3-methylbutan-2-yl ((S)-4-methyl-1-oxo-1-(((S)-1-oxo-3-((S)-2-oxopyrrolidin-3-yl)propan-2-yl)amino)pentan-2-yl)carbamate (A222) (180 mg, 0.314 mmol) in DCM (5 mL) was added isocyanocyclopropane (0.06 mL, 0.944 mmol) and pyridine (0.5 mL, 3 vol) followed by TFA (0.006 mL, 0.062 mmol) at 0° C. The reaction mixture was allowed to stir at RT for 16 h. The progress of the reaction was monitored by TLC and LC-MS. Reaction mixture was diluted with dichloromethane and washed with Satd. NaHCO₃solution (50 mL) and water (50 mL) followed by brine (50 mL). The organic layer was dried over anhydrous Na₂SO₄ and evaporated under reduced pressure to afford 1-([1,1′-biphenyl]-3-yl)-1,1-difluoro-3-methylbutan-2-yl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (14). TLC system: 10% MeOH in DCM Rf: 0.5 LCMS (ESI): m/z 657.4 [M+H]⁺

### 1-([1,1′-biphenyl]-3-yl)-1,1-difluoro-3-methylbutan-2-yl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (A226)

To a stirred solution 1-([1,1′-biphenyl]-3-yl)-1,1-difluoro-3-methylbutan-2-yl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (14) (170 mg, 0.258 mmol) in ethyl acetate (10 mL) was added Dess-Martin periodinane (164.67 mg, 0.388 mmol) at 0° C. and stirred at RT for 3 h. The progress of the reaction was monitored by TLC and LC-MS. The suspension was filtered through a pad of diatomaceous earth and washed with ethyl acetate. The filtrate was washed with sat. NaHCO₃ solution (3×20 mL) followed by sat. Hypo solution (3×50 mL). The organic layer was dried over anhydrous Na₂SO₄, filtered and concentrated to get material. The compound was purified using reverse phase (buffer: 0.1% ABC/CAN) to afford 1-([1,1′-biphenyl]-3-yl)-1,1-difluoro-3-methylbutan-2-yl((S)-1-(((S)-4-(cyclopropylamino)-3,4-dioxo-1-((S)-2-oxopyrrolidin-3-yl) butan-2-yl)amino)-4-methyl-1-oxopentan-2-yl)carbamate (A226) (15). TLC system: 10% MeOH in DCM Rf: 0.5 LCMS (ESI): m/z 655.4 [M+H]⁺

### Example 16: Synthesis of Compounds A227 and A228

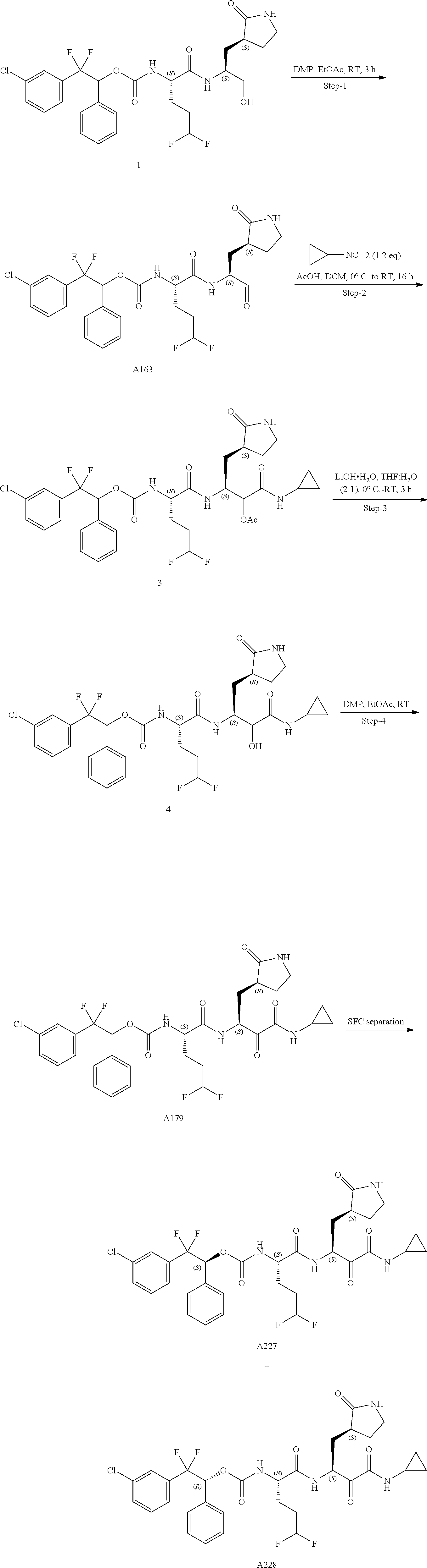

### 2-(3-chlorophenyl)-2,2-difluoro-1-phenylethyl ((S)-5,5-difluoro-1-oxo-1-(((S)-1-oxo-3-((S)-2-oxopyrrolidin-3-yl)propan-2-yl)amino)pentan-2-yl)carbamate (A163)

To a stirred solution of 2-(3-chlorophenyl)-2,2-difluoro-1-phenylethyl ((S)-5,5-difluoro-1-(((S)-1-hydroxy-3-((S)-2-oxopyrrolidin-3-yl)propan-2-yl)amino)-1-oxopentan-2-yl)carbamate (1) (2 g, 3.40 mmol) in Ethyl acetate (30 ml) was added Dess-Martin periodinane (2.9 g, 6.81 mmol) slowly portion wise at 0° C. and stirred at RT for 2 h. After completion of the reaction by TLC, reaction mixture was diluted with Ethyl acetate (40 ml) and filtered through diatomaceous earth pad and washed with Ethyl acetate (20 mL). Obtained filtrate was washed with sat. Hypo solution (3×50 mL) followed by sat. NaHC₃ solution (3×50 mL). Organic layer was separated, dried over anhy. Na₂SO₄, filtered and concentrated under reduced pressure. Obtained compound was purified by trituration with diethyl ether/n-Pentane to afford 2-(3-chlorophenyl)-2,2-difluoro-1-phenylethyl ((S)-5,5-difluoro-1-oxo-1-(((S)-1-oxo-3-((S)-2-oxo pyrrolidin-3-yl)propan-2-yl)amino)pentan-2-yl) carbamate (A163). TLC system: 5% Methanol in DCM Rf: 0.5 LCMS (ESI): m/z 586.2 [M+H]⁺

### (3S)-3-((2S)-2-(((2-(3-chlorophenyl)-2,2-difluoro-1-phenylethoxy)carbonyl)amino)-5,5-difluoropentanamido)-1-(cyclopropylamino)-1-oxo-4-((S)-2-oxopyrrolidin-3-yl)butan-2-yl acetate (3)

To a stirred solution of 2-(3-chlorophenyl)-2,2-difluoro-1-phenylethyl ((S)-5,5-difluoro-1-oxo-1-(((S)-1-oxo-3-((S)-2-oxopyrrolidin-3-yl)propan-2-yl)amino)pentan-2-yl)carbamate (A163) (1.2 g, 2.05 mmol) in dichloromethane (20 mL) was added acetic acid (1 ml) and cyclopropyl isocyanide (2) (687 mg, 10.25 mmol) at 0° C. and stirred at RT for 16 h. Reaction was monitored by TLC and LCMS. After completion of the reaction, reaction mixture was diluted with DCM (20 mL) and washed with water (3×40 mL) and brine solution. Organic layer was separated, dried over anhy. Na₂SO₄, filtered and concentrated under reduced pressure. Obtained residue was purified by combi-flash, and 3% methanol in dichloromethane used as eluent to afford (3S)-3-((2S)-2-(((2-(3-chlorophenyl)-2,2-difluoro-1-phenylethoxy) carbonyl)amino)-5,5-difluoro pentanamido)-1-(cyclo propylamino)-1-oxo-4-((S)-2-oxopyrrolidin-3-yl)butan-2-yl acetate (3). TLC system: 10% Methanol in dichloromethane Rf: 0.4 LCMS (ESI): m/z 713.3 (M+H)⁺

### 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-5,5-difluoro-1-oxopentan-2-yl)carbamate (4)

To a stirred solution of (3S)-3-((2S)-2-(((2-(3-chlorophenyl)-2,2-difluoro-1-phenylethoxy) carbonyl) amino)-5,5-difluoropentanamido)-1-(cyclopropylamino)-1-oxo-4-((S)-2-oxopyrrolidin-3-yl) butan-2-yl acetate (3) (1 g, 1.4 mmol) in THE (8 mL), water (4 mL) was added LiOH·H₂O (0.11 g, 2.8 mmol) at 0° C. and stirred at room temperature for 1 h. The progress of the reaction was monitored by TLC and LCMS. After completion of the reaction, reaction mixture was concentrated under reduced pressure to remove THF. Compound was acidified with aq. 1N HCl solution up to pH˜2 and extracted with ethyl acetate (2×100 mL). Combined organic layer was dried over anhy. Na₂SO₄ and concentrated under reduced pressure to afford 2-(3-chlorophenyl)-2,2-difluoro-1-phenylethyl ((2S)-1-(((2S)-4-(cyclopropyl amino)-3-hydroxy-4-oxo-1-((S)-2-oxopyrrolidine-3-yl)butan-2-yl)amino)-5,5-difluoro-1-oxo pentan-2-yl)carbamate (4). TLC system: 10% Methanol in dichloromethane Rf: 0.3 LCMS (ESI): m/z 671.28 (M+H)⁺

### 2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-5,5-difluoro-1-oxopentan-2-yl)carbamate (A179)

To a stirred solution of 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-5,5-difluoro-1-oxo pentan-2-yl)carbamate (8) (1 g, 1.49 mmol) in Ethyl acetate (20 mL) was added Dess-Martin periodinane (1.2 g, 2.98 mmol) at 0° C. and stirred at RT for 2 h. After completion of the reaction, reaction mixture was diluted with Ethyl acetate (20 mL) and filtered through calcite pad. Obtained filtrate was washed with sat. Hypo solution (3×50 mL) followed by sat. NaHCO₃ solution (3×50 mL). Organic layer was separated, dried over anhydrous Na₂SO₄, filtered and concentrated under reduced pressure. Obtained compound was purified by trituration with n-Pentane/DEE to afford 2-(3-chlorophenyl)-2,2-difluoro-1-phenylethyl ((S)-1-(((S)-4-(cyclopropylamino)-3,4-dioxo-1-((S)-2-oxopyrrolidin-3-yl) butan-2-yl)amino)-5,5-difluoro-1-oxopentan-2-yl)carbamate (A179). TLC system: 5% Methanol in DCM Rf: 0.5 LCMS (ESI): m/z 669.2 [M+H]⁺

### (S)-2-(3-chlorophenyl)-2,2-difluoro-1-phenylethyl ((S)-1-(((S)-4-(cyclopropylamino)-3,4-di oxo-1-((S)-2-oxopyrrolidin-3-yl) butan-2-yl)amino)-5,5-difluoro-1-oxopentan-2-yl) carbamate (A227) and (R)-2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-5,5-difluoro-1-oxopentan-2-yl)carbamate (A228)

Compound 2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-5,5-difluoro-1-oxopentan-2-yl)carbamate (A179) (800 mg) was purified by chiral SFC to afford (S)-2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-5,5-difluoro-1-oxopentan-2-yl)carbamate (A227) and (R)-2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-5,5-difluoro-1-oxopentan-2-yl)carbamate (A228). TLC system: 10% Methanol in DCM Rf: 0.3 LCMS (ESI): m/z 647.3 [M+H]⁺

### Example 17: Synthesis of Compound A229

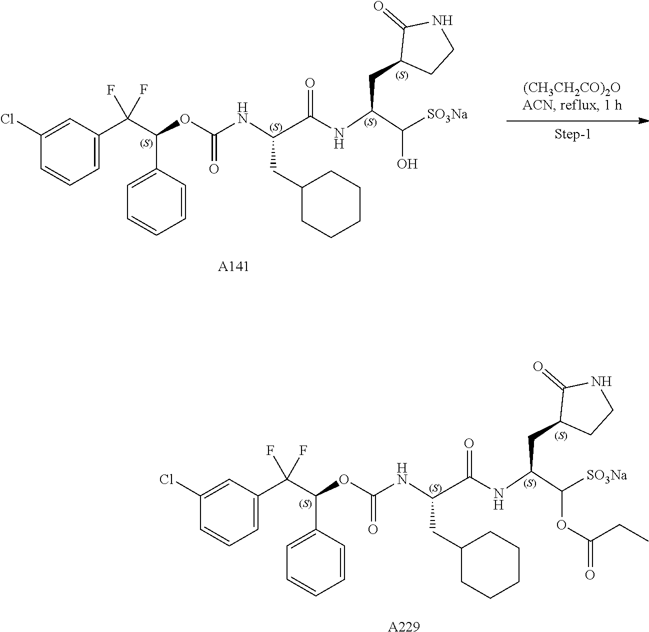

### sodium (2S,6S,9S)-1-(3-chlorophenyl)-6-(cyclohexylmethyl)-1,1-difluoro-4,7,12-trioxo-9-(((S)-2-oxopyrrolidin-3-yl)methyl)-2-phenyl-3,11-dioxa-5,8-diazatetradecane-10-sulfonate (A229)

To a stirred solution of sodium (2S)-2-((S)-2-((((S)-2-(3-chlorophenyl)-2,2-difluoro-1-phenylethoxy)carbonyl)amino)-3-cyclohexylpropanamido)-1-hydroxy-3-((S)-2-oxopyrrolidin-3-yl)propane-1-sulfonate (A141) (200 mg, 0.282 mmol) in ACN (4 mL) was added Propionic anhydride (0.06 g, 0.424 mmol) at RT and stirred at 50° C. for 16 h. The progress of the reaction was monitored by LC-MS. The reaction mixture was concentrated and triturated with pentane (10 mL) and diethyl ether (10 mL) followed by prep. HPLC/lyophilization to afford sodium (2R,6S,9S)-1-(3-chlorophenyl)-6-(cyclohexylmethyl)-1,1-difluoro-4,7,12-trioxo-9-(((S)-2-oxopyrrolidin-3-yl)methyl)-2-phenyl-3,11-dioxa-5,8-diazatetradecane-10-sulfonate (A229). TLC system: 10% Methanol in dichloromethane Rf: 0.2 LCMS (ESI): m/z 742.2 [M−Na]⁺

Prep. HPLC conditions: Column/dimensions: X BIRDGE C18 (19*250, Sum) Mobile phase A: MILI-Q WATER Mobile phase B: Acetonitrile Gradient (Time/% B): 0/10, 1/10, 9/40, 14.9/40, 15/98, 19.9/98, 20/10, 22/10; Flow rate: 18 mL/min. Solubility: acetonitrile.

### Example 18: Synthesis of Compound A230

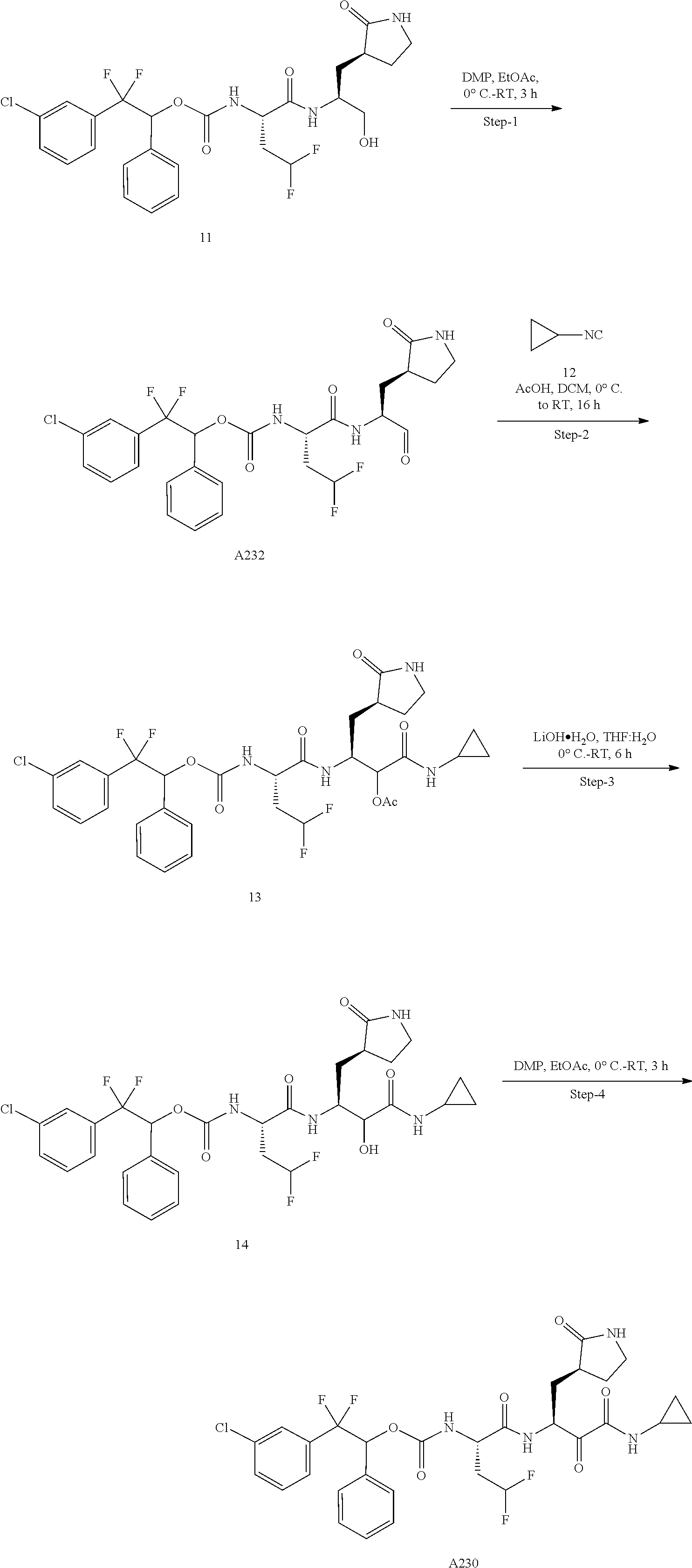

### 2-(3-chlorophenyl)-2,2-difluoro-1-phenylethyl ((S)-4,4-difluoro-1-oxo-1-(((S)-1-oxo-3-((S)-2-oxopyrrolidin-3-yl)propan-2-yl)amino)butan-2-yl)carbamate (A232)

To a stirred solution of 2-(3-chlorophenyl)-2,2-difluoro-1-phenylethyl ((S)-4,4-difluoro-1-(((S)-1-hydroxy-3-((S)-2-oxopyrrolidin-3-yl)propan-2-yl)amino)-1-oxobutan-2-yl)carbamate (11) (700 mg, 1.2 mmol) in ethyl acetate (10 mL) was added Dess-Martin periodinane (1.29 g, 3.05 mmol) slowly portion wise at 0° C. and stirred at RT for 2 h. The progress of the reaction was monitored by TLC and LCMS. Reaction mixture was diluted with Ethyl acetate (20 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure to afford 2-(3-chlorophenyl)-2,2-difluoro-1-phenylethyl ((S)-4,4-difluoro-1-oxo-1-(((S)-1-oxo-3-((S)-2-oxopyrrolidin-3-yl)propan-2-yl)amino)butan-2-yl)carbamate. TLC system: 10% Methanol in DCM R_{f}: 0.55 LCMS (ESI): m/z 572.3 [M+H]⁺

### (3S)-3-((2S)-2-(((2-(3-chlorophenyl)-2,2-difluoro-1-phenylethoxy)carbonyl)amino)-4,4-difluorobutanamido)-1-(cyclopropylamino)-1-oxo-4-((S)-2-oxopyrrolidin-3-yl)butan-2-yl acetate (13)

To a stirred solution of 2-(3-chlorophenyl)-2,2-difluoro-1-phenylethyl ((S)-4,4-difluoro-1-oxo-1-(((S)-1-oxo-3-((S)-2-oxopyrrolidin-3-yl)propan-2-yl)amino)butan-2-yl)carbamate (A232) (550 mg, 0.96 mmol) was dissolved in DCM (8 mL), then added isocyanocyclo propane (13) (200 mg, 2.88 mmol) followed by acetic acid (0.17 mL, 2.88 mmol) at 0° C. and stirred at RT for 6 h. The progress of the reaction was monitored by TLC and LCMS. After completion of starting material, reaction mixture was diluted with dichloromethane and washed with sat. ammonium chloride solution (2×30 mL) followed by brine (1×20 mL). Organic layer was dried over anhydrous Na₂SO₄ and evaporated under reduced pressure. Obtained compound was purified by reverse phase (C18) column chromatography using 55% CAN in 0.1% Aq. formic acid as eluent to afford (3S)-3-((2S)-2-(((2-(3-chlorophenyl)-2,2-difluoro-1-phenylethoxy)carbonyl)amino)-4,4-difluorobutanamido)-1-(cyclopropylamino)-1-oxo-4-((S)-2-oxopyrrolidin-3-yl)butan-2-ylacetate (13). TLC system: 10% MeOH in DCM Rf: 0.5 LCMS (ESI): m/z 699 [M+H]⁺

### 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4,4-difluoro-1-oxobutan-2-yl)carbamate (14)

To a stirred solution of (3S)-3-((2S)-2-(((2-(3-chlorophenyl)-2,2-difluoro-1-phenylethoxy) carbonyl)amino)-4,4-difluorobutanamido)-1-(cyclopropylamino)-1-oxo-4-((S)-2-oxopyrrolidin-3-yl)butan-2-ylacetate (13) (150 mg, 0.107 mmol) in THE (2 mL), water (0.4 mL) was added LiOH·H2O (9.9 mg, 0.236 mmol) at 0° C. and stirred at 0° C. for 30 min. The progress of the reaction was monitored by TLC and LCMS. After completion of the reaction, reaction mixture was extracted with ethyl acetate (2×30 mL). Combined organic layer was washed with brine solution (30 mL), dried over anhydrous sodium sulfate and concentrated under reduced to afford 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4,4-difluoro-1-oxobutan-2-yl)carbamate (14). TLC system: 10% MeOH in DCM Rf: 0.65 LCMS (ESI): m/z 657.41 [M+H]+

### 2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-4,4-difluoro-1-oxobutan-2-yl)carbamate (A230)

To a stirred solution of 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4,4-difluoro-1-oxobutan-2-yl)carbamate (14) (90 mg, 0.132 mmol) in ethyl acetate (2 mL) was added Dess-Martin periodinane (87.2 mg, 0.205 mmol) at 0° C. and stirred at RT for 1 h. The progress of the reaction was monitored by TLC and LCMS. Reaction mixture was filtered through diatomaceous earth bed and washed with ethyl acetate (10 mL). Filtrate was washed with sat. sodium thiosulfate solution (3×20 mL) followed by sat. NaHCO₃ solution (3×20 mL). Organic layer was dried over anhydrous Na₂SO₄, filtered and concentrated. The residue was purified by trituration with n-Penatne/DEE to afford 2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-4,4-difluoro-1-oxobutan-2-yl)carbamate (A230). TLC system: 10% MeOH in DCM Rf: 0.65 LCMS (ESI): m/z 655.2 [M+H]+

### Example 19: Synthesis of Compound A231

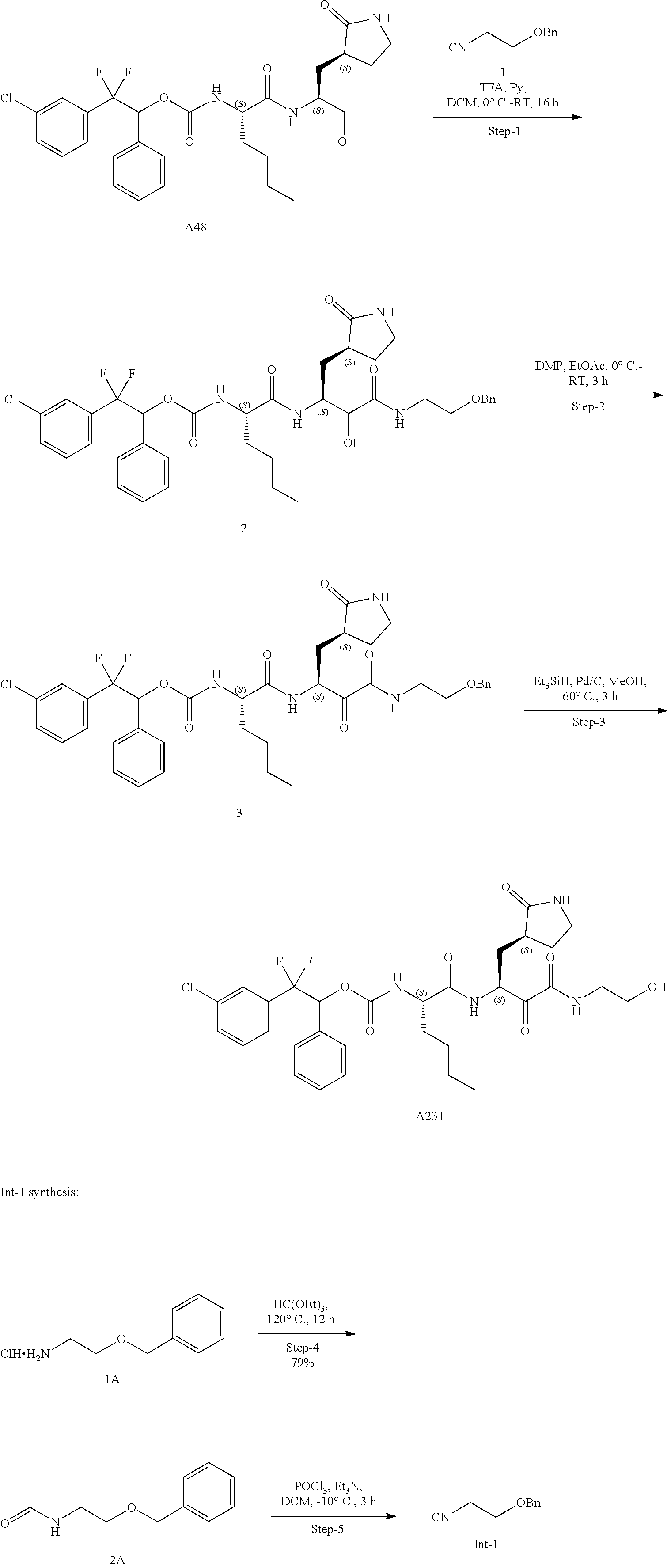

### 2-(3-chlorophenyl)-2,2-difluoro-1-phenylethyl ((2S)-1-(((2S)-4-((2-(benzyloxy)ethyl)amino)-3-hydroxy-4-oxo-1-((S)-2-oxopyrrolidin-3-yl)butan-2-yl)amino)-1-oxohexan-2-yl)carbamate (2)

To a stirred solution of 2-(3-chlorophenyl)-2,2-difluoro-1-phenylethyl ((S)-1-oxo-1-(((S)-1-oxo-3-((S)-2-oxopyrrolidin-3-yl)propan-2-yl)amino)hexan-2-yl)carbamate (A48) (200 mg, 0.3552 mmol) in DCM (6 mL) was added ((2-isocyanoethoxy)methyl)benzene (1) (6 mL, 3 vol), pyridine (6 mL, 3 vol) and TFA (0.01 mL) at 0° C. The reaction mixture was allowed to stir at RT for 16 h. The progress of the reaction was monitored by TLC and LC-MS. Reaction mixture was diluted with dichloromethane (10 mL) and washed with Aq. 1N HCl (3×20 mL) solution followed by brine (1×20 mL). The organic layer was dried over anhydrous Na₂SO₄ and evaporated under reduced pressure to afford 2-(3-chlorophenyl)-2,2-difluoro-1-phenylethyl ((2S)-1-(((2S)-4-((2-(benzyloxy)ethyl)amino)-3-hydroxy-4-oxo-1-((S)-2-oxopyrrolidin-3-yl)butan-2-yl)amino)-1-oxohexan-2-yl)carbamate (2). TLC system: 10% Methanol in dichloromethane Rf: 0.4 LCMS (ESI): m/z 743.34 [M+H]⁺

### 2-(3-chlorophenyl)-2,2-difluoro-1-phenylethyl ((S)-1-(((S)-4-((2-(benzyloxy)ethyl)amino)-3,4-dioxo-1-((S)-2-oxopyrrolidin-3-yl)butan-2-yl)amino)-1-oxohexan-2-yl)carbamate (3)

To a stirred solution of 2-(3-chlorophenyl)-2,2-difluoro-1-phenylethyl ((2S)-1-(((2S)-4-((2-(benzyloxy)ethyl)amino)-3-hydroxy-4-oxo-1-((S)-2-oxopyrrolidin-3-yl)butan-2-yl)amino)-1-oxohexan-2-yl)carbamate (2) (190 mg, 0.2560 mmol) in ethyl acetate (5 mL) was added Dess-Martin periodinane (217 mg, 0.5121 mmol) at 0° C. and stirred at RT for 3 h. progress of the reaction monitored by TLC. The suspension was filtered through a pad of diatomaceous earth and washed with ethyl acetate (20 mL), the filtrate was washed with sat. Hypo solution (3×25 mL) followed by sat. NaHCO₃ solution (3×25 mL) and brine (25 mL). Organic layer was dried over anhydrous Na₂SO₄, and concentrated to afford 2-(3-chlorophenyl)-2,2-difluoro-1-phenylethyl ((S)-1-(((S)-4-((2-(benzyloxy)ethyl)amino)-3,4-dioxo-1-((S)-2-oxopyrrolidin-3-yl)butan-2-yl)amino)-1-oxohexan-2-yl)carbamate (3). TLC system: 10% Methanol in dichloromethane Rf: 0.4 LCMS (ESI): m/z 741.2 (M+H)⁺

### 2-(3-chlorophenyl)-2,2-difluoro-1-phenylethyl ((S)-1-(((S)-4-((2-hydroxyethyl)amino)-3,4-dioxo-1-((S)-2-oxopyrrolidin-3-yl)butan-2-yl)amino)-1-oxohexan-2-yl)carbamate (A231)

To a stirred solution of 2-(3-chlorophenyl)-2,2-difluoro-1-phenylethyl ((S)-1-(((S)-4-((2-(benzyloxy)ethyl)amino)-3,4-dioxo-1-((S)-2-oxopyrrolidin-3-yl)butan-2-yl)amino)-1-oxohexan-2-yl)carbamate (3) (170 mg, 0.2297 mmol) in MeOH (6 mL) was added 10% Pd/C (34 mg, 20% w/w) and triethylsilane (80 mg, 0.6891 mmol) at RT. The reaction mixture was stirred at 60° C. for 3 h. After completion, the reaction mixture was filtered through a pad of diatomaceous earth. The filtrate was dried over Na₂SO₄ and concentrated to get compound. The material was purified by prep. HPLC to afford 2-(3-chlorophenyl)-2,2-difluoro-1-phenylethyl ((S)-1-(((S)-4-((2-hydroxyethyl)amino)-3,4-dioxo-1-((S)-2-oxopyrrolidin-3-yl)butan-2-yl)amino)-1-oxohexan-2-yl)carbamate (A231). TLC system: 10% Methanol in dichloromethane Rf: 0.4

Prep. HPLC conditions: Column/dimensions: KROMOSIL (25*150,) 10 um Mobile Phase A: 10 mM ABC in water (aq) Mobile phase B: 10 Mm ABC (1:1) ACN AND MeOH Gradient (Time/% B): 0/40, 1/40, 18/98, 22/98, 22.1/40, 25/40 Flow rate: 25 mL/min. Solubility: ACN+THF+WATER, LCMS (ESI): m/z 651.2 (M+H)⁺

### Int-1-Synthesis: N-(2-(benzyloxy)ethyl)formamide

To a mixture of 2-(benzyloxy)ethan-1-amine hydrochloride (1A) in triethyl orthoformate (2 g, 10.689 mmol) at RT. The reaction mixture was allowed to stir at 120° C. for 12 h. After completion, the reaction mixture was concentrated under vacuum to afford N-(2-(benzyloxy)ethyl)formamide (2A). TLC system: 5% Methanol in dichloromethane Rf: 0.3 LCMS (ESI): m/z 180.2 (M+H)⁺

### ((2-Isocyanoethoxy)methyl)benzene

To a stirred solution of N-(2-(benzyloxy)ethyl)formamide (2A) (1.5 g, 8.379 mmol) in DCM (4.5 mL), was added POCl₃ (0.5 mL, 6.284 mmol), Et₃N (2.1 mL, 20.93 mmol) at −10° C. The reaction mixture was allowed to stir at −10° C. for 3 h. After completion, the reaction mixture was quenched with sat. NaHCO₃ solution (10 mL) and extracted with DCM (2×10 mL). The combined organic layer was washed with water (2×15 mL) followed by brine (1×15 mL), dried over sodium sulfate to get ((2-isocyanoethoxy)methyl)benzene (Int-1) (10 mL). TLC system: 5% Methanol in dichloromethane Rf: 0.3 LCMS (ESI): m/z 161.99 (M+H)⁺

### Example 20: Synthesis of Compound A232

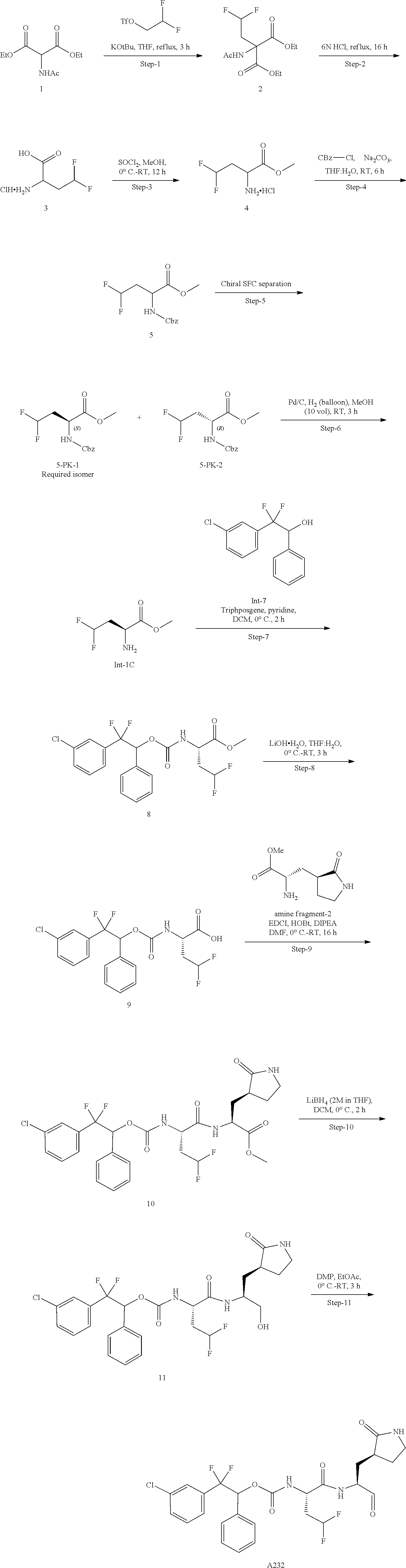

### Diethyl 2-acetamido-2-(2,2-difluoroethyl)malonate (2)

To a stirred solution of diethyl 2-acetamidomalonate (1) (50 g, 230 mmol) in THE (1000 mL) was added KOtBu (25.8 g, 230 mmol) and refluxed for 2 h. After that added 2,2-difluoroethyl trifluoromethanesulfonate (74 g, 345 mmol) slowly drop wise at 70° C. and stirred at same temperature for 3 h. The progress of the reaction was monitored by TLC and LCMS. After completion of starting material, reaction mixture was concentrated and obtained residue was diluted with EtOAc (500 mL) and washed with 0.5N HCl (2×500 mL), 1N aq·NaOH (2×500 mL) and brine solution (500 mL). Organic layer was separated, dried over anhydrous sodium sulfate and evaporated under reduced pressure. The residue was purified by re-crystallization using DEE at −20° C. and filtered the solid to afford Diethyl 2-acetamido-2-(2,2-difluoroethyl)malonate (2). TLC system: 50% Ethyl acetate in hexane Rf: 0.6 LCMS (ESI): m/z 282.3 [M+H]⁺

### 2-amino-4,4-difluorobutanoic acid hydrochloride (3)

To Diethyl 2-acetamido-2-(2,2-difluoroethyl)malonate (2) (25 g, 88.9 mmol) was added 6N HCl (250 mL) at RT and mixture was refluxed at 100° C. for 16 h. Reaction progress was monitored by TLC. After completion of starting material, reaction mass was washed with DEE and aq. layer was concentrated under reduced pressure to afford 2-amino-4,4-difluorobutanoic acid hydrochloride (3). TLC system: 50% Ethyl acetate in hexane Rf: 0.1 LCMS (ESI): m/z 140.15 [M+H]⁺

### methyl 2-amino-4,4-difluorobutanoate hydrochloride (4)

To a stirred solution of 2-amino-4,4-difluorobutanoic acid hydrochloride (3) (11 g, 63.2 mmol) in Methanol (110 mL) was added SOCl₂ (55 mL) slowly drop wise at 0° C. and stirred at RT for 12 h. The progress of the reaction was monitored by LCMS. After completion of starting material, reaction mixture was directly concentrated under reduced pressure to afford methyl 2-amino-4,4-difluorobutanoate hydrochloride (4). LCMS (ESI): m/z 154.13 [M+H]⁺

### methyl 2-(((benzyloxy)carbonyl)amino)-4,4-difluorobutanoate (5)

To a stirred solution of 2-amino-4,4-difluorobutanoate hydrochloride (4) (11 g, 58.02 mmol) in THE (66 mL) and water (33 ml) was added NaHCO₃ (14.8 g, 175.5 mmol) at RT followed by Cbz-Cl (15 g, 87.7 mmol) slowly drop wise at 0° C. and stirred at RT for 6 h. The progress of the reaction was monitored by TLC and LCMS. After completion of starting material, reaction mixture was diluted with ice cold water (300 mL) and extracted with EtOAc (500 mL).

Organic layer was separated, washed with brine solution (300 mL), dried over anhydrous sodium sulfate and evaporated under reduced pressure. Obtained residue was purified by combi-flash column and at 15% ethyl acetate in pet ether as eluent to afford methyl 2-(((benzyloxy)carbonyl)amino)-4,4-difluorobutanoate (5). LCMS (ESI): m/z 288.1 [M+H]⁺

### methyl (S)-2-(((benzyloxy)carbonyl)amino)-4,4-difluorobutanoate (5-PK-1)

15 g of methyl 2-(((benzyloxy)carbonyl)amino)-4,4-difluorobutanoate (5) was purified by chiral SFC to afford methyl (S)-2-(((benzyloxy)carbonyl)amino)-4,4-difluorobutanoate.

### methyl (S)-2-amino-4,4-difluorobutanoate (Int-1C)

To the degassed solution of methyl (S)-2-(((benzyloxy)carbonyl)amino)-4,4-difluorobutanoate (5-PK-1) (3.5 g 12.1 mmol) by nitrogen in methanol (35 mL) was added Pd/C (350 mg) at RT and stirred for 3 h under balloon H₂ pressure. Progress of the reaction was monitored by TLC and LCMS. After completion of starting material, reaction mass was filtered through diatomaceous earth pad and bed washed with methanol and obtained filtrate was concentrated under reduced pressure to afford methyl (S)-2-amino-4,4-difluorobutanoate (Int-1C).

### methyl (2S)-2-(((2-(3-chlorophenyl)-2,2-difluoro-1-phenylethoxy)carbonyl)amino)-4,4-difluorobutanoate (8)

To a stirred solution of 2-(3-chlorophenyl)-2,2-difluoro-1-phenylethan-1-ol (Int-7) (5 g, 18.6 mmol) in DCM (50 mL) was added pyridine (15 ml), followed by Triphosgene (3.3 g, 11.19 mmol) slowly portion wise at 0° C. and stirred for 30 min at same temperature. then added methyl (S)-2-amino-4,4-difluorobutanoate (Int-1C) (3.4 g, 22.3 mmol) at same temperature and stirred at RT for 2 h. The progress of the reaction was monitored by TLC. After completion of starting material, reaction mixture was quenched with 1N HCl (10 ml) and extracted with DCM (200 ml). Organic layer was washed with sat. aq. sodium bicarbonate solution (200 ml) followed by brine solution (200 mL), dried over anhydrous sodium sulfate and evaporated under reduced pressure. Obtained residue was purified by normal phase silica gel column chromatography by using 5% ethyl acetate in pet. ether as eluent to afford methyl (2S)-2-(((2-(3-chlorophenyl)-2,2-difluoro-1-phenylethoxy)carbonyl)amino)-4,4-difluorobutanoate (8). TLC system: 20% Ethyl acetate/Pet ether Rf: 0.3 LCMS(ESI): m/z 448.07 [M+H]⁺

### (2S)-2-(((2-(3-chlorophenyl)-2,2-difluoro-1-phenylethoxy)carbonyl)amino)-4,4-difluorobutanoic acid (9)

To a stirred solution of methyl (2S)-2-(((2-(3-chlorophenyl)-2,2-difluoro-1-phenylethoxy) carbonyl)amino)-4,4-difluorobutanoate (8) (2.9 g, 6.4 mmol) in THE (15 mL) and water (15 mL) was added LiOH·H₂O (0.8 g, 19.4 mmol) at RT and stirred at room temperature for 2 h. The progress of the reaction was monitored by TLC and LCMS. THE solvent was distilled under reduced pressure, compound was acidified with aq. 1N HCl solution up to pH˜2 and extracted with DCM (2×100 mL), Combined organic layer was washed with water (100 mL), brine solution (150 mL), dried over anhydrous sodium sulfate and concentrated under reduced pressure to afford (2S)-2-(((2-(3-chlorophenyl)-2,2-difluoro-1-phenylethoxy)carbonyl)amino)-4,4-difluorobutanoic acid (9). TLC system: 10% Methanol in dichloromethane Rf: 0.1 LCMS (ESI): m/z 414.22 [M−F]⁺

### methyl (2S)-2-((2S)-2-(((2-(3-chlorophenyl)-2,2-difluoro-1-phenylethoxy)carbonyl)amino)-4,4-difluorobutanamido)-3-((S)-2-oxopyrrolidin-3-yl)propanoate (10)

(2S)-2-(((2-(3-chlorophenyl)-2,2-difluoro-1-phenylethoxy)carbonyl)amino)-4,4-difluorobutanoic acid (9) (2.6 g, 6.0 mmol) in DMF (26 mL) was added EDC·HCl (1.72 g, 9 mmol), HOBt (1.37 g, 9 mmol), DIPEA (3.1 mL, 18 mmol) and methyl (S)-2-amino-3-((S)-2-oxopyrrolidin-3-yl)propanoate hydrochloride (amine fragment-2) (1.6 g, 7.2 mmol) at 0° C. and stirred at room temperature for 16 h. Reaction mixture was diluted with ice water (100 mL) and extracted with ethyl acetate (2×100 mL). Combined organic layer was dried over anhydrous sodium sulphate and evaporated under reduced pressure. Obtained residue was purified by column chromatography using silica gel (100-200 mesh) and 95% of Ethylacetate in Pet.ether to afford methyl (2S)-2-((2S)-2-(((2-(3-chlorophenyl)-2,2-difluoro-1-phenylethoxy)carbonyl)amino)-4,4-difluorobutanamido)-3-((S)-2-oxopyrrolidin-3-yl)propanoate (10). TLC system: 5% MeOH/DCM Rf: 0.45 LCMS (ESI): m/z=602.3 [M+H]⁺

### 2-(3-chlorophenyl)-2,2-difluoro-1-phenylethyl ((S)-4,4-difluoro-1-(((S)-1-hydroxy-3-((S)-2-oxopyrrolidin-3-yl)propan-2-yl)amino)-1-oxobutan-2-yl)carbamate (11)

(2S)-2-((2S)-2-(((2-(3-chlorophenyl)-2,2-difluoro-1-phenylethoxy)carbonyl)amino)-4,4-difluorobutanamido)-3-((S)-2-oxopyrrolidin-3-yl)propanoate (10) (2.4 g, 3.9 mmol, in DCM (30 mL) was added LiBH₄ (2M in THF, 3.98 mL, 7.9 mmol) was added slowly drop wise at 0° C. and stirred for 2 h. The progress of the reaction was monitored by TLC. After completion of starting material, reaction mixture was quenched with saturated NH₄Cl solution and extracted with dichloromethane (2×100 mL). Combined organic layer was washed with water, dried over anhydrous sodium sulfate and concentrated under reduced pressure. Obtained material was purified by reverse phase combiflash column (C18) and 50% ACN in 0.1% aq·TFA as eluent to afford 2-(3-chlorophenyl)-2,2-difluoro-1-phenylethyl ((S)-4,4-difluoro-1-(((S)-1-hydroxy-3-((S)-2-oxopyrrolidin-3-yl) propan-2-yl)amino)-1-oxobutan-2-yl)carbamate (11). TLC system: 5% Methanol in dichloromethane Rf: 0.35 LCMS (ESI): m/z 574.15 [M+H]⁺

### 2-(3-chlorophenyl)-2,2-difluoro-1-phenylethyl ((S)-4,4-difluoro-1-oxo-1-(((S)-1-oxo-3-((S)-2-oxopyrrolidin-3-yl)propan-2-yl)amino)butan-2-yl)carbamate (A232)

To a stirred solution of 2-(3-chlorophenyl)-2,2-difluoro-1-phenylethyl ((S)-4,4-difluoro-1-(((S)-1-hydroxy-3-((S)-2-oxopyrrolidin-3-yl)propan-2-yl)amino)-1-oxobutan-2-yl)carbamate (11) (360 mg, 0.62 mmol) in ethyl acetate (10 mL) was added Dess-Martin periodinane (399 mg, 0.94 mmol) slowly portion wise at 0° C. and stirred at RT for 2 h. The progress of the reaction was monitored by TLC and LCMS. Reaction mixture was diluted with Ethyl acetate (20 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure. Obtained material was purified by reverse phase column (C18) chromatography and 45% CAN in 0.1% Aq. NH₄CO₃ solution to afford 2-(3-chlorophenyl)-2,2-difluoro-1-phenylethyl ((S)-4,4-difluoro-1-oxo-1-(((S)-1-oxo-3-((S)-2-oxopyrrolidin-3-yl)propan-2-yl)amino)butan-2-yl)carbamate (A232). TLC system: 10% Methanol in DCM R_{f}: 0.55 LCMS (ESI): m/z 572.3 [M+H]⁺

### Example 21: Synthesis of Compound A233

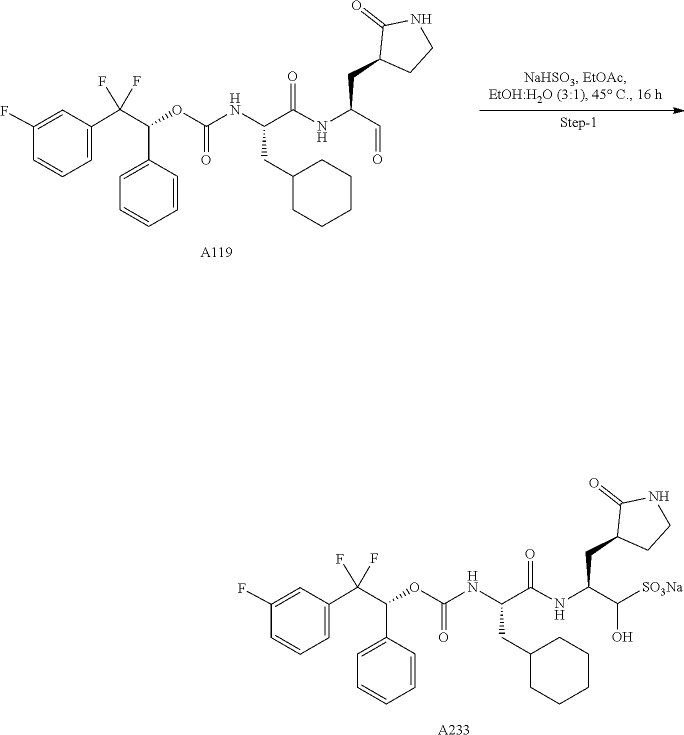

### sodium (2S)-2-((S)-3-cyclohexyl-2-((((R)-2,2-difluoro-2-(3-fluorophenyl)-1-phenylethoxy)carbonyl)amino)propanamido)-1-hydroxy-3-((S)-2-oxopyrrolidin-3-yl)propane-1-sulfonate (A233)

To a stirred solution of (R)-2,2-difluoro-2-(3-fluorophenyl)-1-phenylethyl ((S)-3-cyclohexyl-1-oxo-1-(((S)-1-oxo-3-((S)-2-oxopyrrolidin-3-yl)propan-2-yl)amino)propan-2-yl)carbamate (A119, 150 mg, 0.255 mmol) in Ethyl acetate (1.2 mL), Ethanol (1.35 mL), Water (0.45 mL) was added sodium bisulfate (53 mg; 0.51 mmol) at RT and stirred at 45° C. temperature for 16 h. The progress of the reaction was monitored by TLC and LCMS. After completion of the reaction, reaction mixture was filtered through diatomaceous earth bed and washed with ethyl acetate. Obtained filtrate was dried over anhydrous sodium sulfate and concentrated under reduced pressure. Obtained solid compound was triturated with acetonitrile, Diethyl ether to afford sodium (2S)-2-((S)-3-cyclohexyl-2-((((R)-2,2-difluoro-2-(3-fluorophenyl)-1-phenylethoxy) carbonyl) amino)propanamido)-1-hydroxy-3-((S)-2-oxopyrrolidin-3-yl)propane-1-sulfonate (A233). TLC system: 10% Methanol in Dichloromethane Rf: 0.2 LCMS (ESI): m/z=667.97 [M−Na]⁻

### Example 22: Synthesis of Compound A234

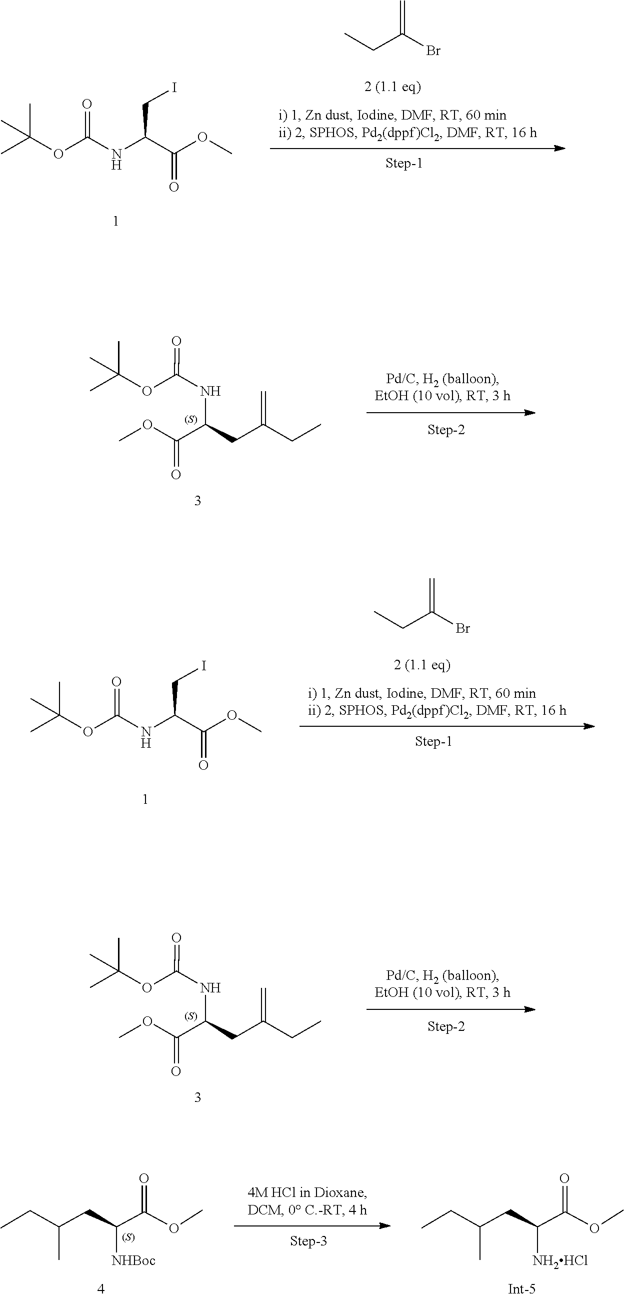

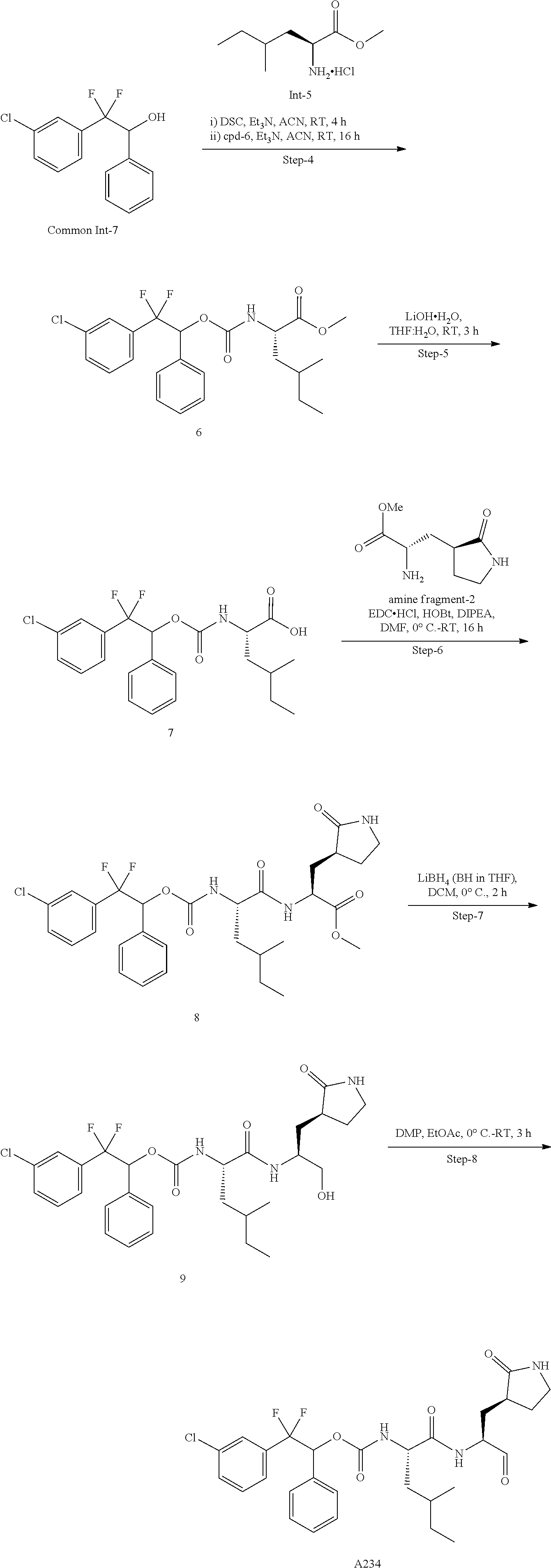

### methyl (S)-2-((tert-butoxycarbonyl)amino)-4-methylenehexanoate (3)

To a suspension of activated zinc (7.75 g, 91.1 mmol) in DMF (50 ml) was added catalytic amount of Iodine at RT and stirred for 10 min and then added methyl (R)-2-((tert-butoxy carbonyl)amino)-3-iodopropanoate (1) (10 g, 30.3 mmol) portionwise at same temperature followed by addition of catalytic amount of iodine and stirred for 60 min at same temperature. Reaction mass degassed with argon balloon for 15 min. and added 2-bromobut-1-ene (2) (4.46 g, 33.3 mmol), Pd₂(dppf)Cl₂ (442.9 mg, 0.60 mmol) and SPHOS (246 mg, 0.60 mmol) at RT and heated to 50° C. for 12 h. The progress of the reaction was monitored by TLC and LCMS. After completion of starting material, reaction mixture was filtered through diatomaceous earth bed and bed was washed with ethyl acetate. Obtained filtrate was diluted with water (250 mL) and extracted with ethyl acetate (2×300 mL). Combined organic layer was washed with brine solution (200 mL), dried over anhydrous sodium sulfate and evaporated under reduced pressure. The residue was purified by column chromatography using silica gel (100-200 mesh) and 0% EtOAc:Pet.Ether as eluent to afford methyl (S)-2-((tert-butoxycarbonyl)amino)-4-methylenehexanoate (3). TLC system: 10% Ethyl acetate in hexane Rf: 0.6 LCMS (ESI): m/z 280.22 [M+Na]⁺

### methyl (2S)-2-((tert-butoxycarbonyl)amino)-4-methylhexanoate (4)

To a degassed solution of methyl (S)-2-((tert-butoxycarbonyl)amino)-4-methylenehexanoate (3) (5.0 g, 19.3 mmol) in EtOH (50 mL) was added Pd/C (1 g, 20% w/w) at RT and stirred for 3 h under H₂ balloon pressure. Reaction progress was monitored by TLC. After completion of starting material, I reaction mass was filtered through diatomaceous earth pad and bed washed with EtOH (30 mL). Obtained filtrate was concentrated under reduced pressure to afford methyl (2S)-2-((tert-butoxycarbonyl)amino)-4-methylhexanoate (4). TLC system: 30% Ethyl acetate in hexane Rf: 0.5 LCMS (ESI): m/z 282.23 [M+Na]⁺

### methyl (2S)-2-amino-4-methylhexanoate hydrochloride (int-5)

To a stirred solution of methyl (2S)-2-((tert-butoxycarbonyl)amino)-4-methylhexanoate (4) (4.0 g, 18.9 mmol) in DCM (24.5 mL) was added 4M HCl in Dioxane (24.5 mL) at 0° C. and stirred at RT for 4 h. The progress of the reaction was monitored by TLC. After completion of starting material, reaction mixture was concentrated under reduced pressure to afford methyl (2S)-2-amino-4-methylhexanoate hydrochloride (int-5). LCMS (ESI): m/z 160.42 [M+H]⁺

### methyl (2S)-2-(((2-(3-chlorophenyl)-2,2-difluoro-1-phenylethoxy)carbonyl)amino)-4-methylhexanoate (6)

To a stirred solution of 2-(3-chlorophenyl)-2,2-difluoro-1-phenylethan-1-ol (Int-7) (8.0 g, 29.8 mmol) in ACN (40 mL) was added N, N′-disuccinamidyl carbonate (19 g, 74.5 mmol) followed by triethylamine (19.4 mL, 150.66 mmol) at 0° C. and stirred the reaction mixture at room temperature for 4 h. The progress of the reaction was monitored by TLC. The reaction mass was used directly in the subsequent reaction.

In another RB flask, afford methyl (2S)-2-amino-4-methylhexanoate hydrochloride (int-5) ACN (40 mL), and treated with triethylamine (19.4 ml, 150.66 mmol). The resulting reaction mixture was stirred for 5 min, then added above prepared reaction mass drop-wise and the reaction mixture was stirred at room temperature for 16 h. Reaction mixture was quenched with ice water (200 mL) and extracted with ethyl acetate (2×250 mL). Combined organic layer was washed with brine solution (200 mL), dried over anhy. Na2SO4 and evaporated under reduced pressure. The was purified by silica gel (100-200 mesh) column chromatography to afford methyl (2S)-2-(((2-(3-chlorophenyl)-2,2-difluoro-1-phenylethoxy)carbonyl)amino)-4-methylhexanoate (6). TLC system: 30% Ethyl acetate in hexane Rf: 0.55 LCMS (ESI): m/z [M+H]⁺

### (2S)-2-(((2-(3-chlorophenyl)-2,2-difluoro-1-phenylethoxy)carbonyl)amino)-4-methyl hexanoic acid (7)

To a stirred solution of methyl (2S)-2-(((2-(3-chlorophenyl)-2,2-difluoro-1-phenyl ethoxy) carbonyl)amino)-4-methylhexanoate (6) (6.8 g, 15.0 mmol) in THE (34 mL) and water (34 mL) was added LiOH·H₂O (1.88 g, 45 mmol) at RT and stirred at room temperature for 2 h. The progress of the reaction was monitored by TLC. THE was distilled under reduced pressure, compound acidified with aq. 1N HCl solution up to pH˜2 and extracted with DCM (2×100 mL). Combined organic layer was washed with water (100 mL) brine solution (150 mL), dried over anhy. sodium sulfate and concentrated under reduced pressure to afford (2S)-2-(((2-(3-chlorophenyl)-2,2-difluoro-1-phenylethoxy)carbonyl)amino)-4-methylhexanoic acid (7). TLC system: 10% Methanol in dichloromethane Rf: 0.1 LCMS (ESI): m/z 440.28 [M+H]⁺

### methyl (2S)-2-((2S)-2-(((2-(3-chlorophenyl)-2,2-difluoro-1-phenylethoxy)carbonyl)amino)-4-methylhexanamido)-3-((S)-2-oxopyrrolidin-3-yl)propanoate (8)

To a solution of (2S)-2-(((2-(3-chlorophenyl)-2,2-difluoro-1-phenylethoxy) carbonyl) amino)-4-methylhexanoic acid (7) (5.0 g, 11.3 mmol) in DMF (25 mL) was added EDC·HCl (3.2 g, 17.0 mmol), HOBt (2.56 g, 17.0 mmol), DIPEA (4.88 mL, 28 mmol) and methyl (S)-2-amino-3-((S)-2-oxopyrrolidin-3-yl)propanoate hydrochloride (amine fragment-2) (3.28 g, 14.8 mmol) at 0° C. and stirred at room temperature for 16 h. Reaction mixture was diluted with ice water (100 mL) and extracted with ethyl acetate (2×100 mL). Combined organic layer was dried over anhydrous sodium sulphate and evaporated under reduced pressure. Obtained residue was purified by column chromatography using silica gel (100-200 mesh) to afford methyl (2S)-2-((2S)-2-(((2-(3-chlorophenyl)-2,2-difluoro-1-phenylethoxy)carbonyl)amino)-4-methylhexanamido)-3-((S)-2-oxopyrrolidin-3-yl)propanoate (8). TLC system: 5% MeOH/DCM Rf: 0.45 LCMS (ESI): m/z=608.41 [M+H]⁺

### 2-(3-chlorophenyl)-2,2-difluoro-1-phenylethyl ((2S)-1-(((S)-1-hydroxy-3-((S)-2-oxopyrrolidin-3-yl)propan-2-yl)amino)-4-methyl-1-oxohexan-2-yl)carbamate (9)

To a solution of methyl (2S)-2-((2S)-2-(((2-(3-chlorophenyl)-2,2-difluoro-1-phenylethoxy)carbonyl)amino)-4-methylhexanamido)-3-((S)-2-oxopyrrolidin-3-yl)propanoate (8) (4.0 g, 6.58 mmol) in DCM (40 mL) was added LiBH₄ (2M in THF, 6.58 mL, 13.1 mmol) slowly drop wise at 0° C. and stirred for 2 h at 0° C. The progress of the reaction was monitored by TLC. After completion of starting material, reaction mixture was quenched with saturated NH₄Cl solution and extracted with dichloromethane (2×100 mL). Combined organic layer was washed with water, dried over anhydrous sodium sulfate and concentrated under reduced pressure and purified by reverse phase column (C18) eluting with 50% ACN in 0.1% FA in water to afford 2-(3-chlorophenyl)-2,2-difluoro-1-phenylethyl ((2S)-1-(((S)-1-hydroxy-3-((S)-2-oxopyrrolidin-3-yl)propan-2-yl)amino)-4-methyl-1-oxohexan-2-yl)carbamate (9). TLC system: 5% Methanol in dichloromethane Rf: 0.35 LCMS (ESI): m/z 580.27 [M+H]⁺

### 2-(3-chlorophenyl)-2,2-difluoro-1-phenylethyl ((2S)-4-methyl-1-oxo-1-(((S)-1-oxo-3-((S)-2-oxopyrrolidin-3-yl)propan-2-yl)amino)hexan-2-yl)carbamate (A234)

To a stirred solution of 2-(3-chlorophenyl)-2,2-difluoro-1-phenylethyl ((2S)-1-(((S)-1-hydroxy-3-((S)-2-oxopyrrolidin-3-yl)propan-2-yl)amino)-4-methyl-1-oxohexan-2-yl)carbamate (9) (400 mg, 0.69 mmol) in ethyl acetate (4 mL) was added Dess-Martin periodinane (585 mg, 1.39 mmol) slowly portion wise at 0° C. and stirred at RT for 2 h. The progress of the reaction was monitored by TLC and LCMS. Reaction mixture was diluted with Ethyl acetate (50 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure. Obtained material was purified by reverse phase combiflash column (C18) using 50% ACN in 0.1% aq. NH₄CO₃ solution as eluent to afford 2-(3-chlorophenyl)-2,2-difluoro-1-phenylethyl ((2S)-4-methyl-1-oxo-1-(((S)-1-oxo-3-((S)-2-oxopyrrolidin-3-yl)propan-2-yl)amino)hexan-2-yl)carbamate (A234). TLC system: 10% Methanol in DCM R_{f}: 0.55 LCMS (ESI): m/z 578.3 [M+H]⁺

### Example 23: Synthesis of Compound A235

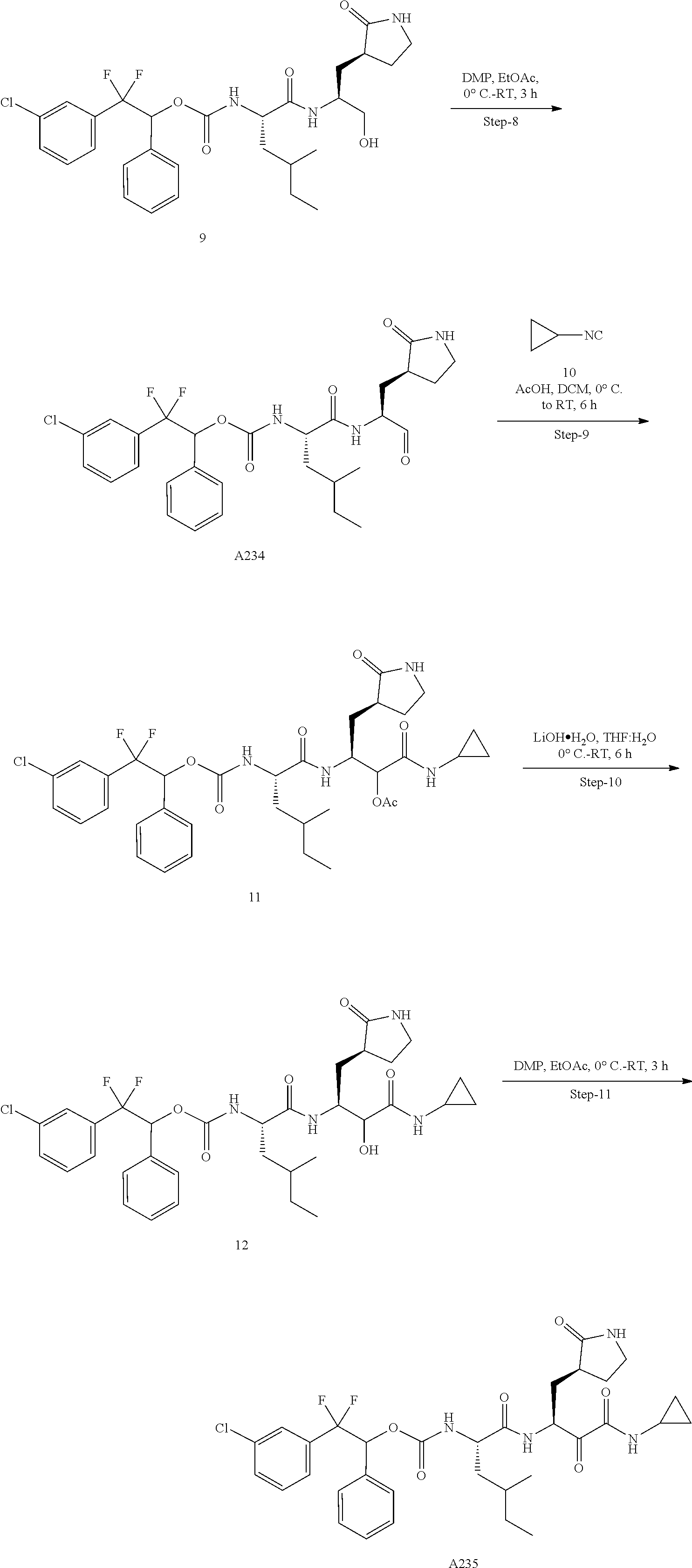

### 2-(3-chlorophenyl)-2,2-difluoro-1-phenylethyl ((2S)-4-methyl-1-oxo-1-(((S)-1-oxo-3-((S)-2-oxopyrrolidin-3-yl)propan-2-yl)amino)hexan-2-yl)carbamate (A234)

To a stirred solution of 2-(3-chlorophenyl)-2,2-difluoro-1-phenylethyl ((2S)-1-(((S)-1-hydroxy-3-((S)-2-oxopyrrolidin-3-yl)propan-2-yl)amino)-4-methyl-1-oxohexan-2-yl)carbamate (9) (0.7 g, 1.20 mmol) in ethyl acetate (7 mL) was added Dess-Martin periodinane (1.02 g 2.41 mmol) slowly portion wise at 0° C. and stirred at RT for 2 h. The progress of the reaction was monitored by TLC and LCMS. Reaction mixture was diluted with Ethyl acetate (50 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure to afford 2-(3-chlorophenyl)-2,2-difluoro-1-phenylethyl ((2S)-4-methyl-1-oxo-1-(((S)-1-oxo-3-((S)-2-oxopyrrolidin-3-yl)propan-2-yl)amino)hexan-2-yl)carbamate (A234) which was used directly in the next step. TLC system: 10% Methanol in DCM R_{f}: 0.55 LCMS (ESI): m/z 578.3 [M+H]⁺

### (3S)-3-((2S)-2-(((2-(3-chlorophenyl)-2,2-difluoro-1-phenylethoxy)carbonyl)amino)-4-methylhexanamido)-1-(cyclopropylamino)-1-oxo-4-((S)-2-oxopyrrolidin-3-yl)butan-2-yl acetate (11)

To a stirred solution of 2-(3-chlorophenyl)-2,2-difluoro-1-phenylethyl ((2S)-4-methyl-1-oxo-1-(((S)-1-oxo-3-((S)-2-oxopyrrolidin-3-yl)propan-2-yl)amino)hexan-2-yl)carbamate (A234) (0.48 g, 0.83 mmol) in DCM (4.8 mL) was added isocyanocyclo propane (10) (160 mg, 2.49 mmol) followed by acetic acid (0.15 mL, 2.49 mmol) at 0° C. and stirred at RT for 6 h. The progress of the reaction was monitored by TLC and LCMS. After completion of starting material reaction mixture was diluted with dichloromethane and washed with sat. ammonium chloride solution (2×30 mL) followed by brine (1×20 mL). Organic layer was dried over anhydrous Na₂SO₄ and evaporated under reduced pressure.

Obtained material was purified by reverse phase combi flash column (C18) using 50% ACN in 0.1% FA in water as eluent to afford (3S)-3-((2S)-2-(((2-(3-chlorophenyl)-2,2-difluoro-1-phenylethoxy)carbonyl)amino)-4-methylhexa namido)-1-(cyclopropylamino)-1-oxo-4-((S)-2-oxopyrrolidin-3-yl)butan-2-yl acetate (11). TLC system: 10% MeOH in DCM Rf: 0.5 LCMS (ESI): m/z 705.33 [M+H]+

### 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4-methyl-1-oxohexan-2-yl)carbamate (12)

To a stirred solution of (3S)-3-((2S)-2-(((2-(3-chlorophenyl)-2,2-difluoro-1-phenylethoxy) carbonyl)amino)-4-methylhexanamido)-1-(cyclopropylamino)-1-oxo-4-((S)-2-oxopyrrolidin-3-yl)butan-2-yl acetate (11 (350 mg, 0.49 mmol) in THE (3.1 mL) and water (1.05 mL) was added LiOH·H₂O (31.2 mg, 0.74 mmol) at 0° C. and stirred at room temperature for 1 h. The progress of the reaction was monitored by TLC and LCMS. After completion of the reaction, reaction mixture was extracted with ethyl acetate (2×50 mL). Combined organic layer was washed with brine solution (30 mL), dried over anhy. sodium sulfate and concentrated under reduced pressure to afford 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4-methyl-1-oxohexan-2-yl) carbamate (12). TLC system: 10% MeOH in DCM Rf: 0.4 LCMS (ESI): m/z 663.4 [M+H]+

### 2-(3-chlorophenyl)-2,2-difluoro-1-phenylethyl ((2S)-1-(((S)-4-(cyclopropylamino)-3,4-dioxo-1-((S)-2-oxopyrrolidin-3-yl)butan-2-yl)amino)-4-methyl-1-oxohexan-2-yl)carbamate (A235)

To a stirred solution of afford 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-4-methyl-1-oxohexan-2-yl)carbamate (12) (250 mg, 0.37 mmol) in ethyl acetate (2.5 mL) was added Dess-Martin periodinane (320.2 mg, 0.75 mmol) at 0° C. and stirred at RT for 1 h. The progress of the reaction was monitored by TLC and LCMS. Reaction mixture was filtered through diatomaceous earth bed and washed with ethyl acetate (10 mL). Obtained filtrate was washed with sat. sodium thiosulfate solution (3×20 mL) followed by sat. NaHCO₃ solution (3×20 mL). Organic layer was dried over anhydrous Na₂SO₄, filtered and concentrated under reduced pressure. Obtained residue was purified by trituration with n-Penatne/DEE to afford 2-(3-chlorophenyl)-2,2-difluoro-1-phenylethyl ((2S)-1-(((S)-4-(cyclopropylamino)-3,4-dioxo-1-((S)-2-oxopyrrolidin-3-yl)butan-2-yl)amino)-4-methyl-1-oxohexan-2-yl)carbamate (A235). TLC system: 10% MeOH in DCM Rf: 0.65 LCMS (ESI): m/z 661.3 [M+H]+

### Example 24: Synthesis of Compounds A236 and A237

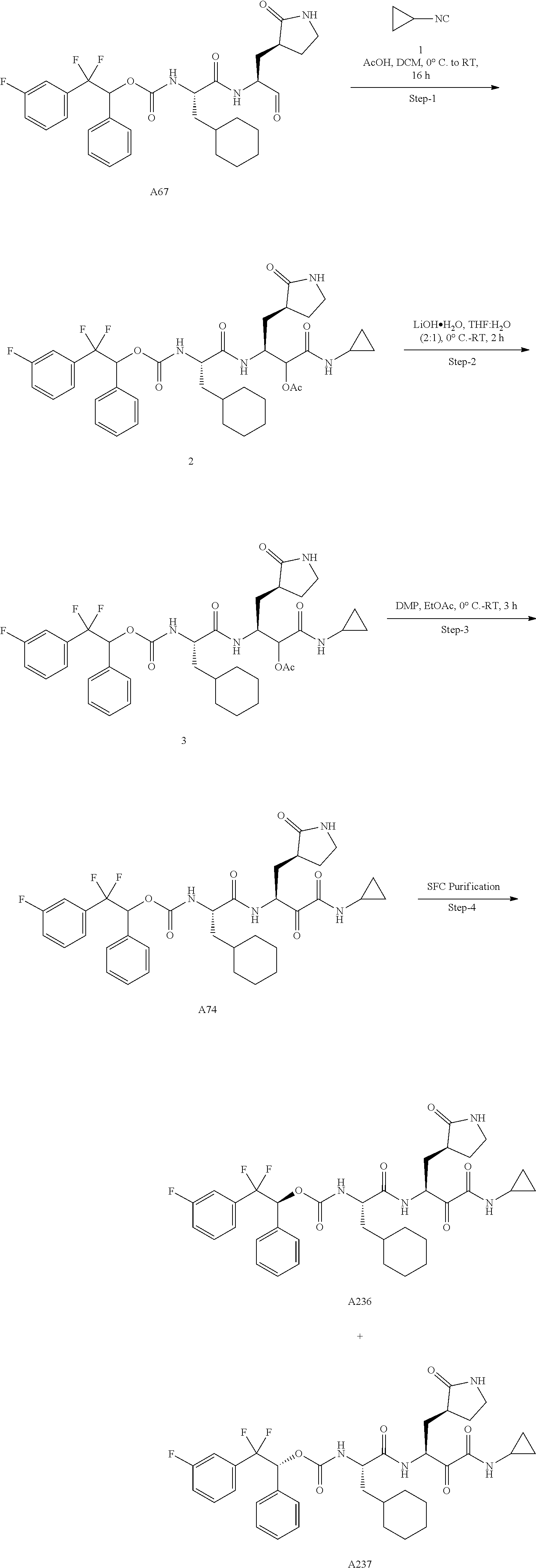

### (3S)-3-((2S)-3-cyclohexyl-2-(((2,2-difluoro-2-(3-fluorophenyl)-1-phenylethoxy)carbonyl) amino)propanamido)-1-(cyclopropylamino)-1-oxo-4-((S)-2-oxopyrrolidin-3-yl)butan-2-yl acetate (2)

To a stirred solution of 2,2-difluoro-2-(3-fluorophenyl)-1-phenylethyl ((S)-3-cyclohexyl-1-oxo-1-(((S)-1-oxo-3-((S)-2-oxopyrrolidin-3-yl)propan-2-yl)amino)propan-2-yl)carbamate (A67) (600 mg, 1.02 mmol) in dichloromethane (6 mL) was added acetic acid (0.18 ml, 3.06 mmol) and cyclopropyl isocyanide (1) (200 mg, 3.06 mmol) at 0° C. and stirred at RT for 2 h. Reaction was monitored by TLC and LCMS. After completion of the reaction, reaction mixture was diluted with DCM (20 mL) and washed with water (3×40 mL) and brine solution. Organic layer was separated, dried over anhy.

Na₂SO₄, filtered and concentrated under reduced pressure. Obtained residue was purified by reverse phase combi-flash (C18) and 45% of ACN/0.1% FA in water, and 3% methanol in dichloromethane used as eluent to afford (3S)-3-((2S)-3-cyclohexyl-2-(((2,2-difluoro-2-(3-fluorophenyl)-1-phenylethoxy)carbonyl) amino)propanamido)-1-(cyclopropylamino)-1-oxo-4-((S)-2-oxopyrrolidin-3-yl)butan-2-yl acetate (2). TLC system: 10% Methanol in Ethyl acetate Rf: 0.4 LCMS (ESI): m/z 715.5 (M+H)⁺

### 2,2-difluoro-2-(3-fluorophenyl)-1-phenylethyl ((2S)-3-cyclohexyl-1-(((2S)-4-(cyclopropyl amino)-3-hydroxy-4-oxo-1-((S)-2-oxopyrrolidin-3-yl)butan-2-yl)amino)-1-oxopropan-2-yl)carbamate (3)

To a stirred solution of (3S)-3-((2S)-3-cyclohexyl-2-(((2,2-difluoro-2-(3-fluorophenyl)-1-phenylethoxy)carbonyl) amino)propanamido)-1-(cyclopropylamino)-1-oxo-4-((S)-2-oxopyrrolidin-3-yl)butan-2-yl acetate (2) (450 mg, 0.63 mmol) in THE (3.6 mL), water (0.9 mL) was added LiOH·H₂O (52 mg, 1.26 mmol) at 0° C. and stirred at same temperature for 1 h. The progress of the reaction was monitored by TLC and LCMS. After completion of the reaction, reaction mixture was concentrated under reduced pressure to remove THF. Compound was acidified with aq. 1N HCl solution up to pH˜2 and extracted with ethyl acetate (2×100 mL). Combined organic layer was dried over anhy. Na₂SO₄ and concentrated under reduced pressure to afford 2,2-difluoro-2-(3-fluorophenyl)-1-phenylethyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (3). TLC system: 10% Methanol in Ethyl acetate Rf: 0.3 LCMS (ESI): m/z 673.92 (M+H)⁺

### 2,2-difluoro-2-(3-fluorophenyl)-1-phenylethyl ((S)-3-cyclohexyl-1-(((S)-4-(cyclopropyl amino)-3,4-dioxo-1-((S)-2-oxopyrrolidin-3-yl)butan-2-yl)amino)-1-oxopropan-2-yl) carbamate (A74)

To a stirred solution of 2,2-difluoro-2-(3-fluorophenyl)-1-phenylethyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (3) (350 mg, 0.52 mmol) in Ethyl acetate (5 mL) was added Dess-Martin periodinane (440 mg, 1.04 mmol) at 0° C. and stirred at RT for 2 h. After completion of the reaction, reaction mixture was diluted with Ethyl acetate (20 mL) and filtered through calcite pad. Obtained filtrate was washed with sat. Hypo solution (3×20 mL) followed by sat. NaHCO₃ solution (3×20 mL). Organic layer was separated, dried over anhydrous Na₂SO₄, filtered and concentrated under reduced pressure. Obtained compound was purified by trituration with n-Pentane/DEE to afford 2,2-difluoro-2-(3-fluorophenyl)-1-phenylethyl ((S)-3-cyclohexyl-1-(((S)-4-(cyclopropyl amino)-3,4-dioxo-1-((S)-2-oxopyrrolidin-3-yl)butan-2-yl)amino)-1-oxo propan-2-yl) carbamate (A74). TLC system: 5% Methanol in ethyl acetate Rf: 0.5 LCMS (ESI): m/z 671.5 [M+H]⁺

### (S)-2,2-difluoro-2-(3-fluorophenyl)-1-phenylethyl ((S)-3-cyclohexyl-1-(((S)-4-(cyclopropyl amino)-3,4-dioxo-1-((S)-2-oxopyrrolidin-3-yl)butan-2-yl)amino)-1-oxopropan-2-yl)carbamate (A236) and (R)-2,2-difluoro-2-(3-fluorophenyl)-1-phenyl ethyl ((S)-3-cyclohexyl-1-(((S)-4-(cyclopropylamino)-3,4-dioxo-1-((S)-2-oxopyrrolidin-3-yl) butan-2-yl)amino)-1-oxopropan-2-yl)carbamate (A237)

Compound 2,2-difluoro-2-(3-fluorophenyl)-1-phenylethyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (A74) (250 mg) was purified by chiral SFC to afford (S)-2,2-difluoro-2-(3-fluorophenyl)-1-phenylethyl ((S)-3-cyclohexyl-1-(((S)-4-(cyclopropyl amino)-3,4-dioxo-1-((S)-2-oxopyrrolidin-3-yl)butan-2-yl)amino)-1-oxopropan-2-yl)carbamate (A236) and (R)-2,2-difluoro-2-(3-fluorophenyl)-1-phenyl ethyl ((S)-3-cyclohexyl-1-(((S)-4-(cyclopropylamino)-3,4-dioxo-1-((S)-2-oxopyrrolidin-3-yl) butan-2-yl)amino)-1-oxopropan-2-yl)carbamate (A237). TLC system: 10% Methanol in DCM Rf: 0.3 LCMS (ESI): m/z 671.3 [M+H]⁺

### Example 25: Synthesis of Compounds A234, A235, A236 and A237

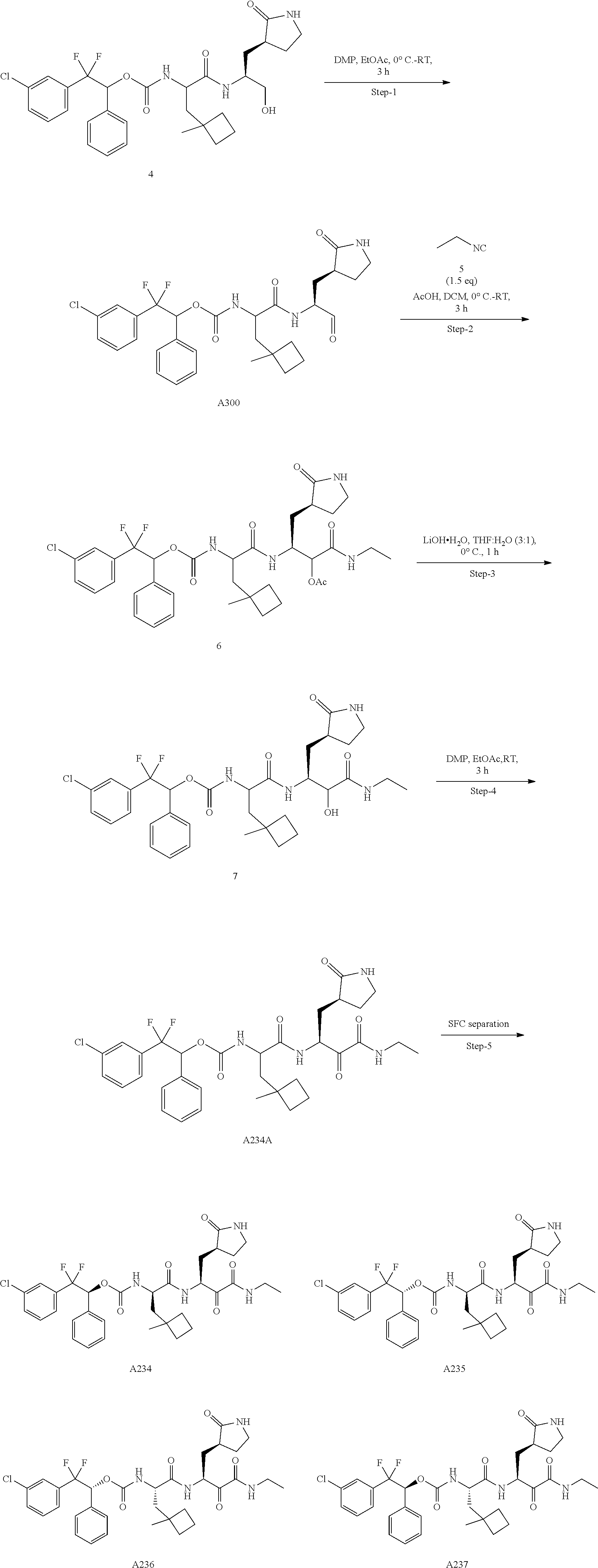

### 2-(3-chlorophenyl)-2,2-difluoro-1-phenylethyl (3-(1-methylcyclobutyl)-1-oxo-1-(((S)-1-oxo-3-((S)-2-oxopyrrolidin-3-yl)propan-2-yl)amino)propan-2-yl)carbamate (A300)

To a stirred solution of 2-(3-chlorophenyl)-2,2-difluoro-1-phenylethyl (1-(((S)-1-hydroxy-3-((S)-2-oxopyrrolidin-3-yl)propan-2-yl)amino)-3-(1-methylcyclobutyl)-1-oxopropan-2-yl) carbamate (4) (4.5 g, 7.6 mmol) in EtOAc (45 mL) was added Dess-Martin periodinane (6.4 g, 15.2 mmol) slowly portion wise at 0° C. and stirred at RT for 3 h. The progress of the reaction was monitored by TLC and LCMS. Reaction mixture was diluted with Ethyl acetate (100 mL), filtered through diatomaceous earth pad and filtrate was washed with sat. sodium thiosulfate solution (3×50 mL) followed by sat. NaHCO₃ solution (3×50 mL). Organic layer was dried over anhydrous Na₂SO₄, filtered and concentrated under reduced pressure. Obtained material was triturated with n-pentane and filtered to afford 2-(3-chlorophenyl)-2,2-difluoro-1-phenylethyl (3-(1-methyl cyclobutyl)-1-oxo-1-(((S)-1-oxo-3-((S)-2-oxopyrrolidin-3-yl)propan-2-yl)amino)propan-2-yl) carbamate (A300). TLC system: 10% Methanol in DCM Rf: 0.55 LCMS (ESI): m/z 590.3 [M+H]⁺

### (9S)-1-(3-chlorophenyl)-1,1-difluoro-6-((1-methylcyclobutyl)methyl)-4,7,11-trioxo-9-(((S)-2-oxo pyrrolidin-3-yl)methyl)-2-phenyl-3-oxa-5,8,12-triazatetradecan-10-yl acetate (6)

To a stirred solution of 2-(3-chlorophenyl)-2,2-difluoro-1-phenylethyl (3-(1-methylcyclobutyl)-1-oxo-1-(((S)-1-oxo-3-((S)-2-oxopyrrolidin-3-yl)propan-2-yl)amino)propan-2-yl)carbamate (A300) (3.0 g, 5.01 mmol) in DCM (30 mL) was added isocyanoethane (5) (1.2 g, 25.45 mmol) (Freshly prepared in DCM solution) followed by acetic acid (6 mL) at 0° C. and stirred at RT for 3 h. The progress of the reaction was monitored by TLC and LCMS. After completion of starting material, reaction mixture was diluted with dichloromethane (100 mL) and washed with sat. ammonium chloride solution (2×30 mL) followed by brine (30 mL). Organic layer was dried over anhydrous Na₂SO₄ and evaporated under reduced pressure. Obtained material was purified by reverse phase combi flash column (C18) using 50% ACN in 0.1% FA in water as eluent to afford (9S)-1-(3-chlorophenyl)-1,1-difluoro-6-((1-methylcyclobutyl)methyl)-4,7,11-trioxo-9-(((S)-2-oxopyrrolidin-3-yl)methyl)-2-phenyl-3-oxa-5,8,12-triazatetradecan-10-yl acetate (6). TLC system: 10% Methanol in dichloromethane Rf: 0.4 LCMS (ESI): m/z 705.3 (M+H)⁺

### 2-(3-chlorophenyl)-2,2-difluoro-1-phenylethyl (1-(((2S)-4-(ethylamino)-3-hydroxy-4-oxo-1-((S)-2-oxopyrrolidin-3-yl)butan-2-yl)amino)-3-(1-methylcyclobutyl)-1-oxopropan-2-yl)carbamate (7)

To a stirred solution of afford (9S)-1-(3-chlorophenyl)-1,1-difluoro-6-((1-methylcyclobutyl)methyl)-4,7,11-trioxo-9-(((S)-2-oxopyrrolidin-3-yl)methyl)-2-phenyl-3-oxa-5,8,12-triazatetradecan-10-yl acetate (6) (1.3 g, 1.84 mmol) in THE (13 mL), water (4 mL) was added LiOH·H₂O (92.94 mg, 2.21 mmol) at 0° C. and stirred at same temperature for 1 h The progress of the reaction was monitored by TLC and LCMS. After completion of the reaction, reaction mixture was extracted with ethyl acetate (2×150 mL). Combined organic layer was washed with brine solution (60 mL), dried over anhy. sodium sulfate and concentrated under reduced pressure to afford 2-(3-chlorophenyl)-2,2-difluoro-1-phenylethyl (1-(((2S)-4-(ethylamino)-3-hydroxy-4-oxo-1-((S)-2-oxopyrrolidin-3-yl)butan-2-yl)amino)-3-(1-methylcyclobutyl)-1-oxopropan-2-yl)carbamate (7). TLC system: 10% Methanol in dichloromethane Rf: 0.3 LCMS (ESI): m/z 663.45 (M+H)⁺

### 2-(3-chlorophenyl)-2,2-difluoro-1-phenylethyl (1-(((S)-4-(ethylamino)-3,4-dioxo-1-((S)-2-oxopyrrolidin-3-yl)butan-2-yl)amino)-3-(1-methylcyclobutyl)-1-oxopropan-2-yl)carbamate (A234A)

To a stirred solution of 2-(3-chlorophenyl)-2,2-difluoro-1-phenylethyl (1-(((2S)-4-(ethylamino)-3-hydroxy-4-oxo-1-((S)-2-oxopyrrolidin-3-yl)butan-2-yl)amino)-3-(1-methylcyclobutyl)-1-oxopropan-2-yl)carbamate (7) (0.9 g, 1.35 mmol) in ethyl acetate (9 mL) was added Dess-Martin periodinane (1.15 g, 2.71 mmol) at 0° C. and stirred at RT for 3 h. After completion of the reaction, reaction mixture was filtered through diatomaceous earth bed and filtrate was washed with sat. Hypo solution (3×100 mL), sat. NaHCO₃ solution (3×70 mL). Organic layer was dried over anhydrous Na₂SO₄ and concentrated under reduced pressure. Obtained compound was purified by trituration with n-Pentane/Et₂O to afford 2-(3-chlorophenyl)-2,2-difluoro-1-phenylethyl (1-(((S)-4-(ethylamino)-3,4-dioxo-1-((S)-2-oxopyrrolidin-3-yl)butan-2-yl) amino)-3-(1-methylcyclobutyl)-1-oxopropan-2-yl)carbamate (A234A). TLC system: 10% MeOH in DCM Rf: 0.65 LCMS (ESI): m/z 661.3 [M+H]⁺

(S)-2-(3-chlorophenyl)-2,2-difluoro-1-phenylethyl ((R)-1-(((S)-4-(ethylamino)-3,4-dioxo-1-((S)-2-oxopyrrolidin-3-yl)butan-2-yl)amino)-3-(1-ethylcyclobutyl)-1-oxopropan-2-yl)carbamate (A234), (R)-2-(3-chlorophenyl)-2,2-difluoro-1-phenylethyl ((R)-1-(((S)-4-(ethylamino)-3,4-dioxo-1-((S)-2-oxopyrrolidin-3-yl)butan-2-yl)amino)-3-(1-ethylcyclobutyl)-1-oxopropan-2-yl)carbamate (A235), (R)-2-(3-chlorophenyl)-2,2-difluoro-1-phenylethyl ((S)-1-(((S)-4-(ethylamino)-3,4-dioxo-1-((S)-2-oxopyrrolidin-3-yl)butan-2-yl)amino)-3-(1-ethylcyclobutyl)-1-oxopropan-2-yl)carbamate (A236), (S)-2-(3-chlorophenyl)-2,2-difluoro-1-phenylethyl ((S)-1-(((S)-4-(ethylamino)-3,4-dioxo-1-((S)-2-oxopyrrolidin-3-yl)butan-2-yl)amino)-3-(1-ethylcyclobutyl)-1-oxopropan-2-yl)carbamate (A237): 0.6 g of 2-(3-chlorophenyl)-2,2-difluoro-1-phenylethyl (1-(((S)-4-(ethylamino)-3,4-dioxo-1-((S)-2-oxopyrrolidin-3-yl)butan-2-yl)amino)-3-(1-methylcyclobutyl)-1-oxopropan-2-yl)carbamate (A234A) was purified by chiral SFC to afford (S)-2-(3-chlorophenyl)-2,2-difluoro-1-phenylethyl ((R)-1-(((S)-4-(ethylamino)-3,4-dioxo-1-((S)-2-oxopyrrolidin-3-yl)butan-2-yl)amino)-3-(1-methylcyclobutyl)-1-oxopropan-2-yl)carbamate (A234), (R)-2-(3-chlorophenyl)-2,2-difluoro-1-phenylethyl ((R)-1-(((S)-4-(ethylamino)-3,4-dioxo-1-((S)-2-oxopyrrolidin-3-yl)butan-2-yl)amino)-3-(1-methylcyclobutyl)-1-oxopropan-2-yl)carbamate (A235), (R)-2-(3-chlorophenyl)-2,2-difluoro-1-phenylethyl ((S)-1-(((S)-4-(ethylamino)-3,4-dioxo-1-((S)-2-oxopyrrolidin-3-yl) butan-2-yl) amino)-3-(1-methylcyclobutyl)-1-oxopropan-2-yl) carbamate (A236) and (S)-2-(3-chlorophenyl)-2,2-difluoro-1-phenylethyl ((S)-1-(((S)-4-(ethylamino)-3,4-dioxo-1-((S)-2-oxopyrrolidin-3-yl)butan-2-yl)amino)-3-(1-methyl cyclobutyl)-1-oxopropan-2-yl)carbamate (A237). LCMS (ESI): m/z 661.3 [M+H]⁺

### Example 26: Synthesis of Compound A238

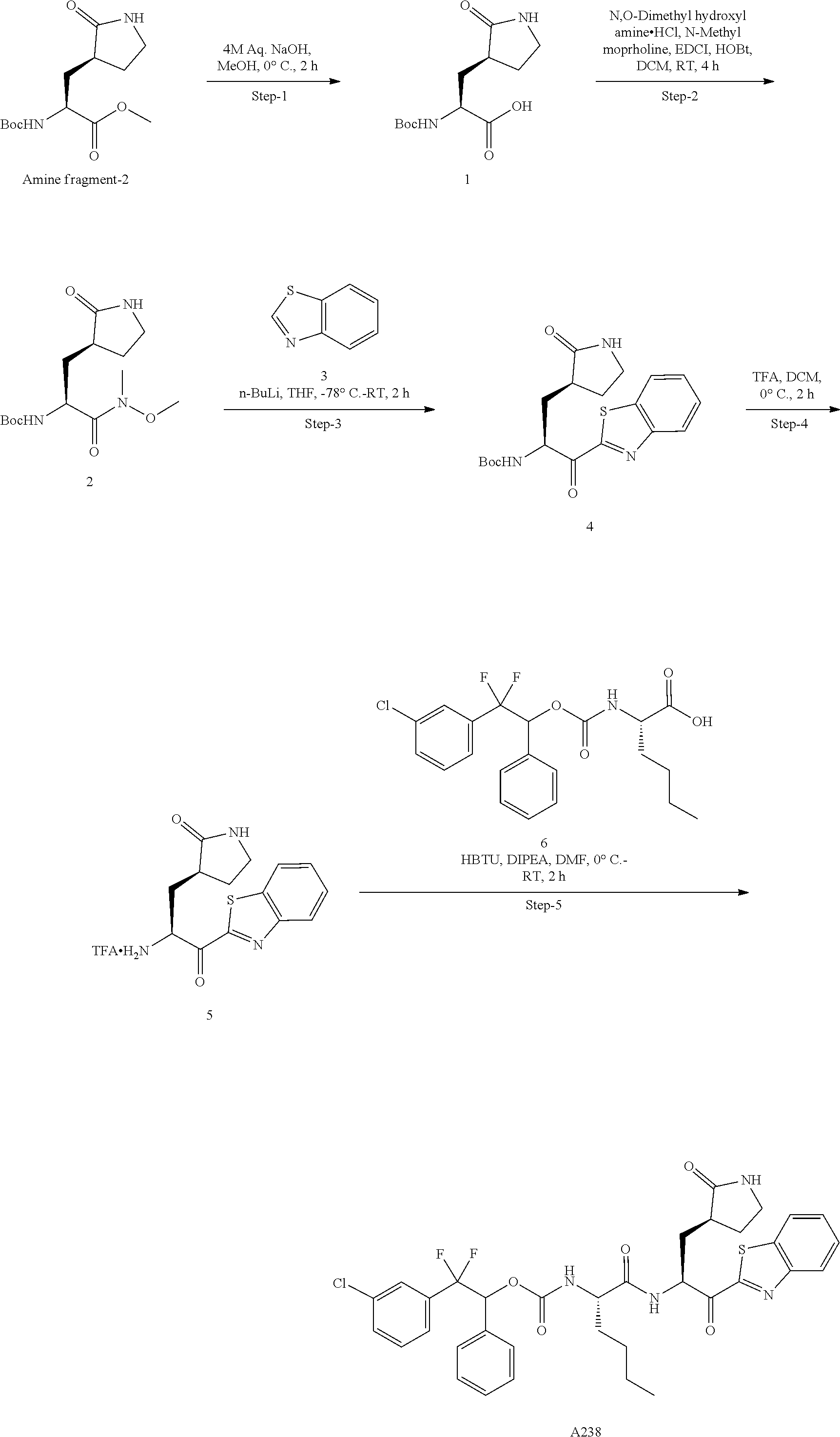

### (S)-2-((tert-butoxycarbonyl)amino)-3-((S)-2-oxopyrrolidin-3-yl)propanoic acid (1)

To a stirred solution of methyl (S)-2-((tert-butoxycarbonyl)amino)-3-((S)-2-oxopyrrolidin-3-yl)propanoate (Amine fragment-2) (15 g, 52.44 mmol) in MeOH (30 mL) at −10° C. was added NaOH (8.39, 262.23 mmol) dissolved in 80 mL of water and added drop wise without raise in internal temperature. The resulting reaction mixture was stirred at −5° C. for 1 h. After completion of the reaction (monitored by TLC), the reaction mixture was evaporated under reduced pressure to get residue material was diluted with water (200 mL), acidified with (up to pH˜2) 1N HCl at 0° C. and extracted with EtOAc (3×200 mL). Combined organic layer was washed with brine solution (200 mL), dried over anhy.

Na₂SO₄ and evaporated under reduced pressure to afford (S)-2-((tert-butoxycarbonyl)amino)-3-((S)-2-oxopyrrolidin-3-yl)propanoic acid (2). TLC system: 10% Methanol in Dichloromethane LCMS (ESI): m/z=173.011 (M+H)⁺ Rf: 0.5

### tert-butyl ((S)-1-(methoxy(methyl)amino)-1-oxo-3-((S)-2-oxopyrrolidin-3-yl)propan-2-yl) carbamate (2)

To a stirred solution of (S)-2-((tert-butoxycarbonyl)amino)-3-((S)-2-oxopyrrolidin-3-yl)propanoic acid (2) (14 g, 51.47 mmol, 1.0 eq) in DCM (140 mL) at 0° C. was added EDCl (10.81 g, 56.61 mmol, 1.1 eq), HOBt (6.94 g, 51.47 mmol, 1.0 eq), N-methyl morpholine (16.95 ml, 154.41 mmol, 3.0 eq) followed by N,O-Dimethyl hydroxyl amine·HCl (4.99 g, 51.47 mmol, 1.0 eq). The resulting reaction mixture was stirred at RT for 4 h. After completion of the reaction (monitored by TLC and LC-MS), the reaction mixture was diluted with water (300 mL) and extracted with DCM (2×250 mL). Organic layer was separated, dried over anhy. Na₂SO₄ and concentrated under reduced pressure. was purified by silica gel (100-200) column chromatography using 0-5% methanol in DCM as eluent to afford tert-butyl ((S)-1-(methoxy(methyl)amino)-1-oxo-3-((S)-2-oxopyrrolidin-3-yl)propan-2-yl)carbamate (3). TLC system: 10% Methanol in Dichloromethane LCMS (ESI): m/z=216.092 (M+H)⁺ Rf: 0.5

### tert-butyl ((S)-1-(benzo[d]thiazol-2-yl)-1-oxo-3-((S)-2-oxopyrrolidin-3-yl)propan-2-yl)carbamate (4)

To a stirred solution of tert-butyl ((S)-1-(methoxy(methyl)amino)-1-oxo-3-((S)-2-oxopyrrolidin-3-yl)propan-2-yl)carbamate (2) (0.5 g, 1.58 mmol)) in THE (5 mL) was added n-BuLi (8.4 ml, 15.87 mmol) at −78° C. and stirred for 30 min. After that added benzo[d]thiazole (3) (1.64 ml, 15.87 mmol) at −78° C. and stirred at −60 to −50° C. for 3 h. The progress of the reaction was monitored by TLC and LCMS. After completion of starting material, reaction mixture was quenched with sat. NH₄Cl (50 mL) and extracted with ethyl acetate (2×100 mL). Organic layer was separated, dried over anhy. Na₂SO₄ and concentrated under reduced pressure. Material was purified by silica gel (100-200 mesh) column chromatography using 0-5% MeOH in DCM as eluent to afford tert-butyl ((S)-1-(benzo[d]thiazol-2-yl)-1-oxo-3-((S)-2-oxopyrrolidin-3-yl)propan-2-yl)carbamate (4). TLC system: 10% Methanol in Dichloromethane Rf: 0.4 LCMS (ESI): m/z=412.12 (M+Na)⁺

### (S)-3-((S)-2-amino-3-(benzo[d]thiazol-2-yl)-3-oxopropyl)pyrrolidin-2-one as TFA salt (5)

To a stirred tert-butyl ((S)-1-(benzo[d]thiazol-2-yl)-1-oxo-3-((S)-2-oxopyrrolidin-3-yl) propan-2-yl)carbamate (4) (0.5 g, 1.28 mmol) in DCM (5 mL) was added TFA (3 mL) at 0° C. and stirred at RT for 1 h. The progress of the reaction was monitored by TLC and LCMS. Reaction mixture completely distilled under reduced pressure to afford (S)-3-((S)-2-amino-3-(benzo [d]thiazol-2-yl)-3-oxopropyl)pyrrolidin-2-one as TFA salt (5). TLC system: 10% MeOH/DCM Rf: 0.2 LCMS (ESI): m/z 290.13 [M+H]⁺

### 2-(3-chlorophenyl)-2,2-difluoro-1-phenylethyl ((S)-1-(((S)-1-(benzo[d]thiazol-2-yl)-1-oxo-3-((S)-2-oxopyrrolidin-3-yl)propan-2-yl)amino)-1-oxohexan-2-yl)carbamate (A238)

To a stirred solution of (S)-3-((S)-3-(benzo[d]thiazol-2-yl)-3-oxo-2-((2,2,2-trifluoroacetyl)-14-azaneyl)propyl)pyrrolidin-2-one (3) (0.25 g, 0.62 mmol) in DMF (10 mL) at 0° C. was added (2S)-2-(((2-(3-chlorophenyl)-2,2-difluoro-1-phenylethoxy)carbonyl)amino) hexanoic acid (0.26 g, 0.62 mmol), HBTU (0.23 g, 0.62 mmol, 1.0 eq), DIPEA (0.33 mL, 1.86 mmol) and resulting reaction mixture was stirred at RT for 2 h. After completion of the reaction (monitored by TLC and LC-MS), the reaction mixture was diluted with water (50 mL) and extracted with EtOAc (2×25 mL). The combined organic layer was washed with brine solution (50 mL), dried over anhy. Na₂SO₄. Obtained material was purified by prep HPLC to afford 2-(3-chlorophenyl)-2,2-difluoro-1-phenylethyl ((S)-1-(((S)-1-(benzo[d]thiazol-2-yl)-1-oxo-3-((S)-2-oxopyrrolidin-3-yl)propan-2-yl)amino)-1-oxohexan-2-yl)carbamate (A238). TLC system: 10% MeOH/DCM Rf: 0.2 LCMS (ESI): m/z 2697.2 [M+H]⁺

### Example 27: Synthesis of Compound A239

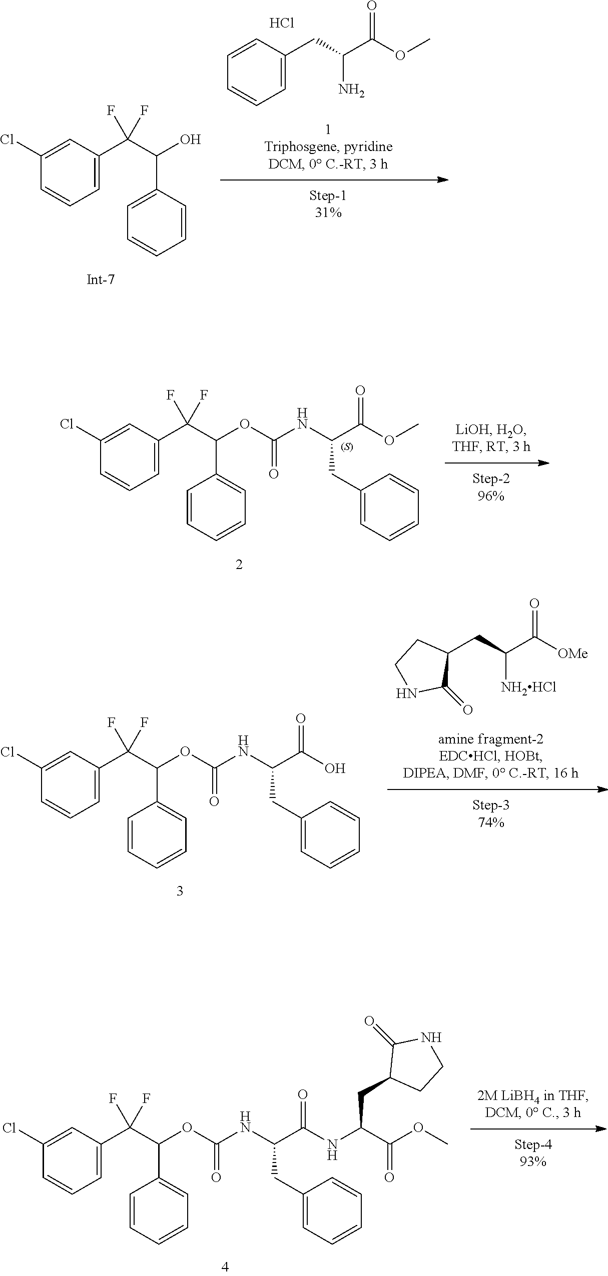

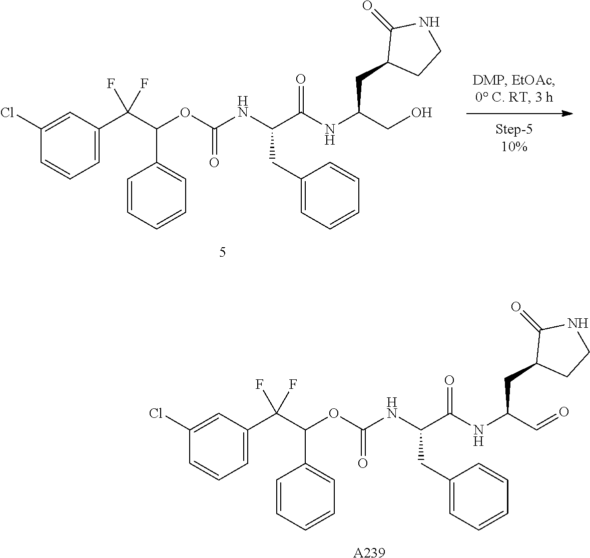 [SMILES: O=C(N[C@@H](Cc1ccccc1)C(=O)N[C@H](CO)C[C@@H]1CCNC1=O)OC(c1ccccc1)C(F)(F)c1cccc(Cl)c1 ; O=C[C@H](C[C@@H]1CCNC1=O)NC(=O)[C@H](Cc1ccccc1)NC(=O)OC(c1ccccc1)C(F)(F)c1cccc(Cl)c1 ; "A239"]

### Methyl ((2-(3-chlorophenyl)-2,2-difluoro-1-phenylethoxy)carbonyl)-L-phenylalaninate (2)

To a stirred solution of 2-(3-chlorophenyl)-2,2-difluoro-1-phenylethan-1-ol (Int-7) (5 g, 18.65 mmol) in DCM (40 mL) was added pyridine (15 mL, 3 vol) and methyl L-phenylalaninate hydrogen chloride (1) (6 g, 27.9 mmol) followed by triphosgene (8.25 g, 27.9 mmol) at 0° C. and stirred at room temperature for 3 h. The progress of the reaction was monitored by TLC and LC-MS. The reaction mixture was quenched with 1N HCl (50 mL) and extracted with DCM (2×40 mL). The combined organic layer was dried over sodium sulfate and evaporated under reduced pressure. The residue was purified by combi-flash, compound eluted at 10% ethyl acetate in pet ether to afford methyl ((2-(3-chlorophenyl)-2,2-difluoro-1-phenylethoxy)carbonyl)-L-phenylalaninate (2). TLC system: 10% Ethyl acetate in hexane Rf: 0.3 LCMS (ESI): m/z 496.1 [M+Na]⁺

### ((2-(3-Chlorophenyl)-2,2-difluoro-1-phenylethoxy)carbonyl)-L-phenylalanine (3)

To a stirred solution of methyl ((2-(3-chlorophenyl)-2,2-difluoro-1-phenylethoxy)carbonyl)-L-phenylalaninate (2) (2.7 g, 5.7 mmol) in THF (20 mL), water (20 mL) was added lithium hydroxide (719 g, 1.71 mmol) at 0° C. and stirred at room temperature for 3 h. The progress of the reaction was monitored by TLC and LC-MS. Solvent was distilled under reduced pressure, compound acidified with aq. 1N HCl solution up to pH˜2 and extracted with ethyl acetate (2×40 mL), dried over sodium sulfate, concentrated under reduced pressure to afford ((2-(3-chlorophenyl)-2,2-difluoro-1-phenylethoxy)carbonyl)-L-phenylalanine (3). TLC system: 10% MeOH in DCM Rf: 0.5 LCMS (ESI): m/z 482.2 [M+Na]⁺

### Methyl (2S)-2-((2S)-2-(((2-(3-chlorophenyl)-2,2-difluoro-1-phenylethoxy)carbonyl)amino)-3-phenylpropanamido)-3-((S)-2-oxopyrrolidin-3-yl)propanoate (4)

To a stirred solution of ((2-(3-chlorophenyl)-2,2-difluoro-1-phenylethoxy)carbonyl)-L-phenylalanine (3) (2 g, 4.35 mmol) in DMF (10 mL) were added EDC·HCl (1.27 g, 6.53 mmol), HOBt (881 mg, 6.53 mmol), DIPEA (2.4 mL, 13.05 mmol) and methyl (S)-2-amino-3-((S)-2-oxopyrrolidin-3-yl) propanoate hydrochloride (amine fragment-2) (1.2 g, 6.53 mmol) at 0° C. The reaction mixture was allowed to stir at room temperature for 16 h. To the reaction mixture, ice water (150 mL) was added and extracted with ethyl acetate (2×50 mL). The combined organic layer was dried over sodium sulfate and evaporated under reduced pressure. The material was purified by combi-flash (normal phase), compound eluted at 3% methanol in dichloromethane to afford methyl (2S)-2-((2S)-2-(((2-(3-chlorophenyl)-2,2-difluoro-1-phenylethoxy)carbonyl)amino)-3-phenyl propanamido)-3-((S)-2-oxopyrrolidin-3-yl)propanoate (4). TLC system: 10% Methanol in dichloromethane Rf: 0.4 LCMS (ESI): m/z 628.3 [M+H]⁺

### 2-(3-Chlorophenyl)-2,2-difluoro-1-phenylethyl((S)-1-(((S)-1-hydroxy-3-((S)-2-oxo pyrrolidin-3-yl) propan-2-yl)amino)-1-oxo-3-phenylpropan-2-yl)carbamate (5)

To a stirred solution of methyl (2S)-2-((2S)-2-(((2-(3-chlorophenyl)-2,2-difluoro-1-phenylethoxy)carbonyl)amino)-3-phenylpropanamido)-3-((S)-2-oxopyrrolidin-3-yl)propanoate (4) (1.7 g, 2.71 mmol) in THE (17 mL) was added 2M LiBH₄ in THE (2.71 mL, 5.42 mmol) at 0° C. and the reaction mixture stirred for 3 h. The progress of the reaction was monitored by TLC. The reaction mixture was quenched with sat. Ammonium chloride solution (30 mL) and extracted with Ethyl acetate (2×50 mL). The combined organic layer was washed with brine (30 mL), dried over Na₂SO₄ and concentrated to afford 2-(3-chlorophenyl)-2,2-difluoro-1-phenylethyl((S)-1-(((S)-1-hydroxy-3-((S)-2-oxopyrrolidin-3-yl)propan-2-yl)amino)-1-oxo-3-phenylpropan-2-yl)carbamate (5). TLC system: 10% Methanol in dichloromethane Rf: 0.2 LCMS (ESI): m/z 600.3 [M+H]⁺

### 2-(3-Chlorophenyl)-2,2-difluoro-1-phenylethyl((S)-1-oxo-1-(((S)-1-oxo-3-((S)-2-oxopyrrolidin-3-yl)propan-2-yl)amino)-3-phenylpropan-2-yl)carbamate (A239)

To a stirred solution of 2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxo-3-phenylpropan-2-yl)carbamate (5) (200 mg, 0.3338 mmol) in ethyl acetate (5 mL) was added Dess-Martin periodinane (283 mg, 0.6677 mmol) at 0° C. portion wise. The reaction mixture was allowed to stir at RT for 3 h. After completion of the reaction by TLC, the reaction mixture was filtered through a pad of diatomaceous earth and washed with ethyl acetate, the filtrate layer was washed with sat. Hypo solution (3×25 mL) followed by sat. NaHCO₃ solution (3×25 mL) and brine (2×25 mL). The organic layer was dried over anhydrous Na₂SO₄, and concentrated to get compound. The material was purified by reverse phase column chromatography to afford 2-(3-chlorophenyl)-2,2-difluoro-1-phenylethyl((S)-1-oxo-1-(((S)-1-oxo-3-((S)-2-oxopyrrolidin-3-yl)propan-2-yl)amino)-3-phenylpropan-2-yl)carbamate. TLC system: 10% Methanol in dichloromethane Rf: 0.5 LCMS (ESI): m/z 598.2 (M+H)⁺

### Example 28: Synthesis of Compound A240

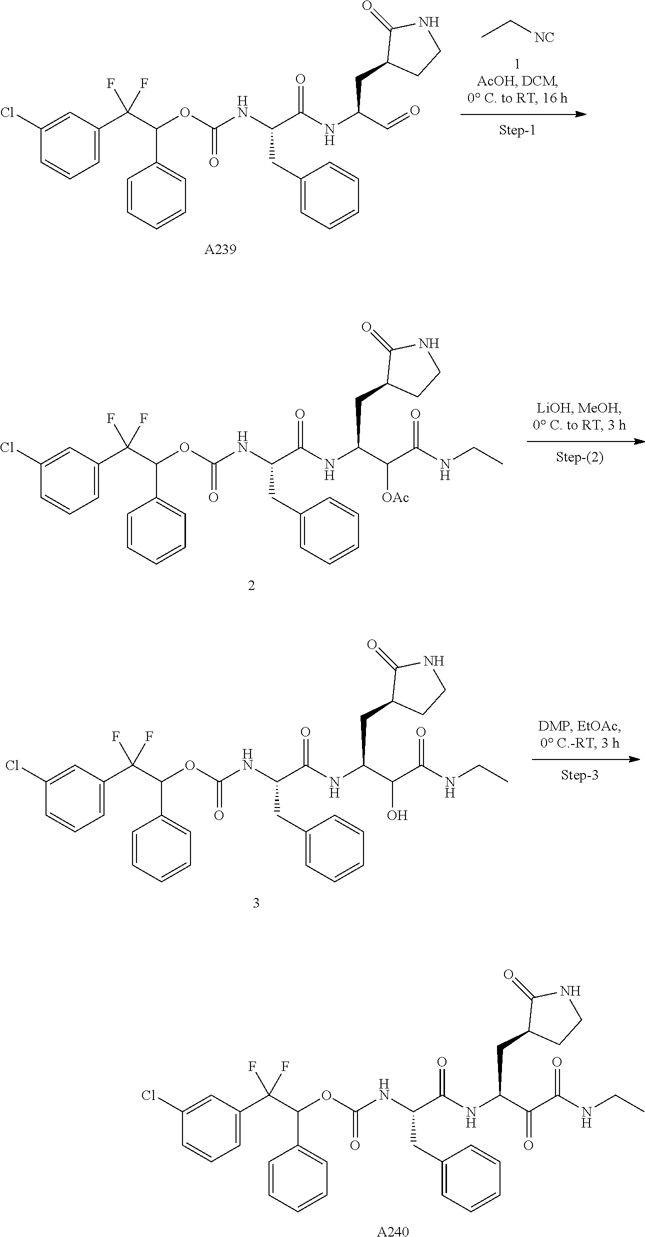

### (6S,9S)-6-benzyl-1-(3-chlorophenyl)-1,1-difluoro-4,7,11-trioxo-9-(((S)-2-oxopyrrolidin-3-yl)methyl)-2-phenyl-3-oxa-5,8,12-triazatetradecan-10-yl acetate (2)

To a stirred solution of 2-(3-chlorophenyl)-2,2-difluoro-1-phenylethyl ((S)-1-oxo-1-(((S)-1-oxo-3-((S)-2-oxopyrrolidin-3-yl)propan-2-yl)amino)-3-phenylpropan-2-yl)carbamate (A239) (200 mg, 0.335 mmol) in DCM (5 mL) was added isocyanoethane (1) (0.7 mL, 0.6700 mmol) followed by Acetic acid (0.2 mL, 0.167 mmol) at 0° C. The reaction mixture was allowed to stir at RT for 16 h. The progress of the reaction was monitored by TLC and LC-MS. The reaction mixture was diluted with dichloromethane and washed with sat. NaHCO₃ (3×25 mL) followed by brine (1×25 mL). The organic layer was dried over anhydrous Na₂SO₄ and evaporated under reduced pressure to afford (6S,9S)-6-benzyl-1-(3-chlorophenyl)-1,1-difluoro-4,7,11-trioxo-9-(((S)-2-oxopyrrolidin-3-yl)methyl)-2-phenyl-3-oxa-5,8,12-triazatetradecan-10-yl acetate (2). TLC system: 10% Methanol in dichloromethane Rf: 0.5 LCMS (ESI): m/z 713.85 [M+H]⁺

### 2-(3-chlorophenyl)-2,2-difluoro-1-phenylethyl ((2S)-1-(((2S)-4-(ethylamino)-3-hydroxy-4-oxo-1-((S)-2-oxopyrrolidin-3-yl)butan-2-yl)amino)-1-oxo-3-phenylpropan-2-yl)carbamate (3)

To a stirred solution of (6S,9S)-6-benzyl-1-(3-chlorophenyl)-1,1-difluoro-4,7,11-trioxo-9-(((S)-2-oxopyrrolidin-3-yl)methyl)-2-phenyl-3-oxa-5,8,12-triazatetradecan-10-yl acetate (2) 210 mg, 0.2949 mmol) in Methanol (5 mL) was added lithium hydroxide (25 mg, 0.5898 mmol) at 0° C. and stirred at room temperature for 3 h. The progress of the reaction was monitored by TLC. Reaction mixture completely distilled under reduced pressure, compound acidified with aq. 1N HCl solution up to pH˜2 and extracted with ethyl acetate (2×25 mL), dried over sodium sulfate, concentrated under reduced pressure to afford 2-(3-chlorophenyl)-2,2-difluoro-1-phenylethyl ((2S)-1-(((2S)-4-(ethylamino)-3-hydroxy-4-oxo-1-((S)-2-oxopyrrolidin-3-yl)butan-2-yl)amino)-1-oxo-3-phenylpropan-2-yl)carbamate (3). TLC system: 10% Methanol in dichloromethane Rf: 0.4 LCMS (ESI): m/z 671.3 (M+H)⁺

### 2-(3-chlorophenyl)-2,2-difluoro-1-phenylethyl ((S)-1-(((S)-4-(ethylamino)-3,4-dioxo-1-((S)-2-oxopyrrolidin-3-yl)butan-2-yl)amino)-1-oxo-3-phenylpropan-2-yl)carbamate (A240)

To a stirred solution of 2-(3-chlorophenyl)-2,2-difluoro-1-phenylethyl ((2S)-1-(((2S)-4-(ethylamino)-3-hydroxy-4-oxo-1-((S)-2-oxopyrrolidin-3-yl)butan-2-yl)amino)-1-oxo-3-phenylpropan-2-yl)carbamate (3) (190 mg, 0.2835 mmol) in ethyl acetate (6 mL) was added Dess-Martin periodinane (240 mg, 0.5671 mmol) at 0° C. and stirred at RT for 3 h. progress of the reaction was monitored by TLC and LC-MS. After completion of the reaction, the suspension was filtered through a pad of diatomaceous earth and washed with ethyl acetate (20 mL). The filtrate was washed with sat. Hypo solution (3×25 mL), sat. NaHCO₃ solution (3×25 mL) and finally brine (3×25 mL). The organic layer was dried over anhydrous Na₂SO₄, and concentrated to get compound. The compound was purified by using reverse phase column chromatography (buffer: 0.1% ABC in water/acetonitrile) to afford 2-(3-chlorophenyl)-2,2-difluoro-1-phenylethyl ((S)-1-(((S)-4-(ethylamino)-3,4-dioxo-1-((S)-2-oxopyrrolidin-3-yl)butan-2-yl)amino)-1-oxo-3-phenylpropan-2-yl)carbamate (A240). TLC system: 10% Methanol in dichloromethane Rf: 0.4 LCMS (ESI): m/z 669.3 (M+H)⁺

### Example 29: Synthesis of Compound A241

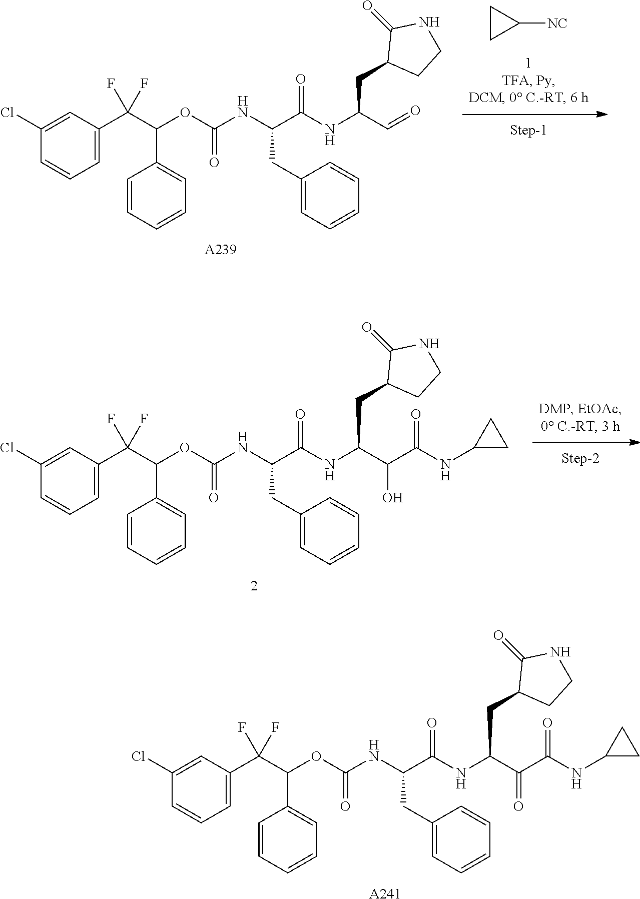

### 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-1-oxo-3-phenylpropan-2-yl)carbamate (2)

To a stirred solution 2-(3-chlorophenyl)-2,2-difluoro-1-phenylethyl ((S)-1-oxo-1-(((S)-1-oxo-3-((S)-2-oxopyrrolidin-3-yl)propan-2-yl)amino)-3-phenylpropan-2-yl)carbamate (A239) (200 mg, 0.3349 mmol) in DCM (5 mL) was added Pyridine (1.2 mL, 6 vol), isocyanocyclopropane (1) (0.04 mL, 0.6700 mmol) followed by TFA (0.01 mL, 0.1675 mmol) at 0° C. and stirred at RT for 6 h. The progress of the reaction was monitored by TLC and LC-MS. The reaction mixture was diluted with dichloromethane and washed with 1N HCl (2×20 mL) followed by brine (1×20 mL). The organic layer was dried over anhydrous Na₂SO₄ and evaporated under reduced pressure to afford 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-1-oxo-3-phenylpropan-2-yl)carbamate (2). TLC system: 10% Methanol in dichloromethane Rf: 0.5 LCMS (ESI): m/z 683.3 [M+H]⁺

### 2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-1-oxo-3-phenylpropan-2-yl)carbamate (A241)

To a stirred solution of 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-1-oxo-3-phenylpropan-2-yl)carbamate (2) (190 mg, 0.278 mmol) in ethyl acetate (5 mL) was added Dess-Martin periodinane (236 mg, 0.5571 mmol) at 0° C. and stirred at RT for 3 h. progress of the reaction monitored by TLC. The suspension was filtered through a pad of diatomaceous earth and washed with ethyl acetate (20 mL), the filtrate was washed with sat. Hypo solution (3×25 mL) followed by sat. NaHCO₃ solution (3×25 mL) and brine (25 mL). The organic layer was dried over anhydrous Na₂SO₄, and concentrated to get compound. The compound was purified with combi-flash (reverse phase) to afford 2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-1-oxo-3-phenylpropan-2-yl)carbamate (A241). TLC system: 10% Methanol in dichloromethane Rf: 0.4 LCMS (ESI): m/z 681.3 (M+H)⁺

### Example 30: Synthesis of Compound A242

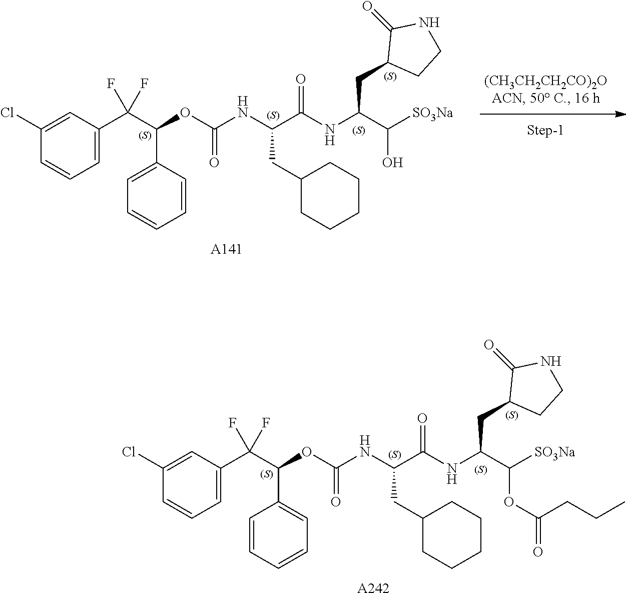

### sodium (2S,6S,9S)-1-(3-chlorophenyl)-6-(cyclohexylmethyl)-1,1-difluoro-4,7,12-trioxo-9-(((S)-2-oxopyrrolidin-3-yl)methyl)-2-phenyl-3,11-dioxa-5,8-diazapentadecane-10-sulfonate (A242)

To a stirred solution of sodium (2S)-2-((S)-2-((((S)-2-(3-chlorophenyl)-2,2-difluoro-1-phenylethoxy)carbonyl)amino)-3-cyclohexylpropanamido)-1-hydroxy-3-((S)-2-oxopyrrolidin-3-yl)propane-1-sulfonate (A141) (200 mg, 0.282 mmol) in ACN (4 mL) was added butyric anhydride (0.06 g, 0.424 mmol) at RT and stirred at 50° C. for 16 h. The progress of the reaction was monitored by LC-MS. The reaction mixture was concentrated to get the material, which was triturated with pentane and diethyl ether followed by prep. HPLC/lyophilization to afford sodium (2S,6S,9S)-1-(3-chlorophenyl)-6-(cyclohexylmethyl)-1,1-difluoro-4,7,12-trioxo-9-(((S)-2-oxopyrrolidin-3-yl)methyl)-2-phenyl-3,11-dioxa-5,8-diazapentadecane-10-sulfonate (A242). TLC system: 20% Methanol in dichloromethane Rf: 0.2 LCMS (ESI): m/z 756.3 [M−Na]⁺

Prep. HPLC conditions: Column/dimensions: KROMOSIL PACKED (25*150 mm) 10 um Mobile Phase A: Miili-Q-Water (aq) Mobile phase B: Acetonitrile Gradient (Time/% B): 0/10, 1/10, 10/40, 15.8/40, 15.81/98, 23.90/98, 24.0/10, 27/10 Flow rate: 24 ml/min. Solubility: ACN+WATER.

### Example 31: Synthesis of Compounds A243 and A243A

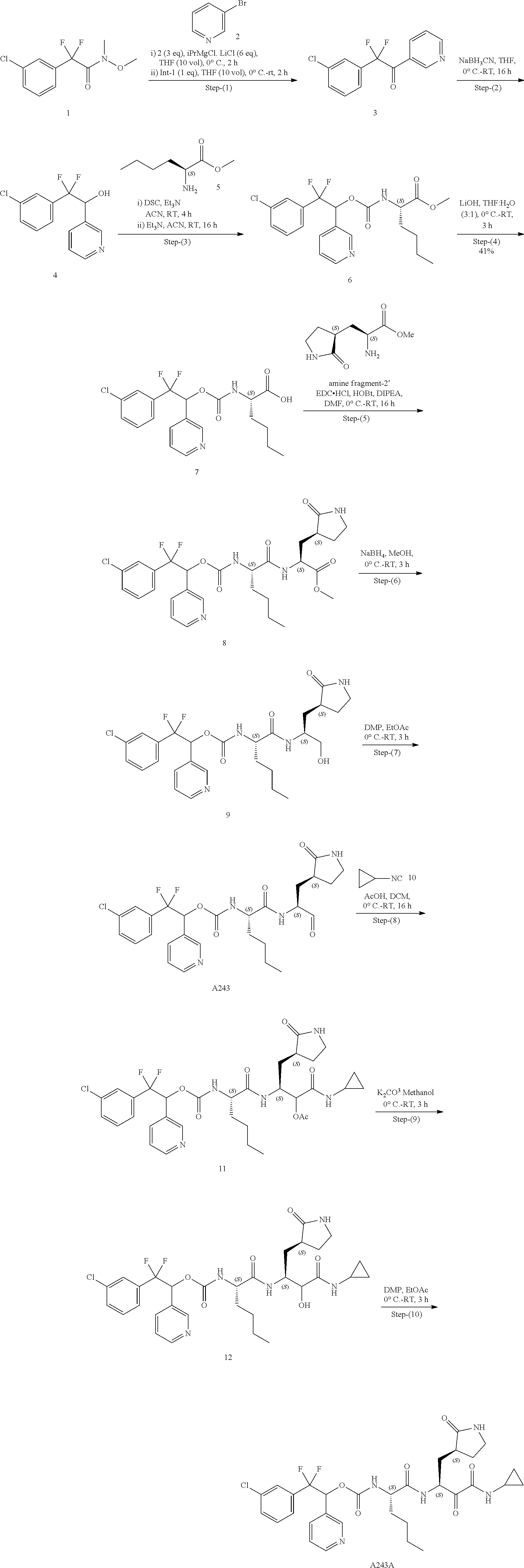

### 2-(3-Chlorophenyl)-2,2-difluoro-1-(pyridin-3-yl)ethan-1-one (3)

To a stirred solution of 3-bromopyridine (2) (5.0 g, 32.051 mmol) in THE was added iPrMgCl·LiCl (50 mL, 64.102 mmol) at −78° C. The reaction mixture was allowed to stir at rt for for 2 h. After 2 h, then was added 2-(3-chlorophenyl)-2,2-difluoro-N-methoxy-N-methylacetamide (1) (8.0 g, 32.051 mmol) at 0° C. and stirred at RT for 3 h. Reaction progress was monitored by TLC. Reaction mixture was quenched with sat. Ammonium chloride solution (100 mL) and extracted with ethyl acetate (2×100 mL). The combined organic layer was washed with water (1×50 mL), brine (1×50 mL), dried over sodium sulfate and evaporated under reduced pressure to afford 2-(3-chlorophenyl)-2,2-difluoro-1-(pyridin-3-yl)ethan-1-one (3). TLC system: 0% Ethyl acetate/Pet ether Rf: 0.5 LCMS (ESI): m/z 286.1 [M+H₂O]⁺

### 2-(3-Chlorophenyl)-2,2-difluoro-1-(pyridin-3-yl)ethan-1-ol (4)

To a stirred solution of 2-(3-chlorophenyl)-2,2-difluoro-1-(pyridin-3-yl)ethan-1-one (3) (6 g, 22.471 mmol) in methanol (60 mL) wad added NaCNBH₃ (5.66 g, 89.887 mmol) at 0° C. The reaction mixture was allowed to stir at RT for 2 h. The reaction mixture was quenched with H₂O (25 mL) and extracted with ethyl acetate (2×50 mL). The combined organic layer was washed with water (1×20 mL), brine (1×20 mL), dried over sodium sulfate and evaporated under reduced pressure to afford 2-(3-chlorophenyl)-2,2-difluoro-1-(pyridin-3-yl)ethan-1-ol (4). TLC system: 30% Ethyl acetate/pet ether Rf: 0.3 LCMS (ESI): m/z 270.2 [M+H]⁺

### Methyl (2S)-2-(((2-(3-chlorophenyl)-2,2-difluoro-1-(pyridin-3-yl)ethoxy)carbonyl)amino)hexanoate (6)

To a stirred solution of 2-(3-chlorophenyl)-2,2-difluoro-1-(pyridin-3-yl)ethan-1-ol (4) (4.2 g, 15.613 mmol) in ACN (42 mL) was added N,N′disuccinamidyl carbonate (9.99 g, 39.033 mmol) followed by Et₃N (6.4 mL, 46.839 mmol) at 0° C. The reaction mixture was allowed to stir at room temperature for 4 h. TLC confirms the formation of non-polar spot. The reaction mass was used directly in the subsequent reaction.

In another RB flask, methyl (S)-2-aminohexanoate hydrochloride (5) (5.65 g, 31.226 mmol) was taken in ACN (56 mL), and treated with Et₃N (6.4 mL, 46.839 mmol). The resulting reaction mixture was stirred for 5 min, then the above prepared reaction mass was added drop-wise and the reaction mixture stirred at room temperature for 16 h.

Reaction mixture was quenched with ice water (50 mL) and extracted with ethyl acetate (2×50 mL), combined organic layers were washed with brine solution (50 mL), dried over sodium sulfate and evaporated under reduced pressure and the residue was purified by normal phase chromatography by using pet ether and ethyl acetate as mobile phases to afford methyl (2S)-2-(((2-(3-chlorophenyl)-2,2-difluoro-1-(pyridin-3-yl)ethoxy)carbonyl)amino)hexanoate (6). TLC system: 20% Ethyl acetate/Pet ether Rf: 0.3 LCMS (ESI): m/z 441.19 [M+H]⁺

### (2S)-2-(((2-(3-Chlorophenyl)-2,2-difluoro-1-(pyridin-3-yl)ethoxy)carbonyl)amino)hexanoic acid (7)

To a stirred solution of methyl (2S)-2-(((2-(3-chlorophenyl)-2,2-difluoro-1-(pyridin-3-yl)ethoxy)carbonyl)amino)hexanoate (6) (4.0 g, 9.090 mmol) in THE (30 mL), water (10 mL) was added lithium hydroxide (654 mg, 27.272 mmol) at RT and stirred at room temperature for 3 h. The progress of the reaction was monitored by TLC. THE was distilled under reduced pressure, compound was acidified with citric acid solution and extracted with ethyl acetate (2×50 mL). The combined organic layer was washed with water (50 mL) brine (50 mL), dried over sodium sulfate and concentrated under reduced pressure to afford (2S)-2-(((2-(3-chlorophenyl)-2,2-difluoro-1-(pyridin-3-yl)ethoxy)carbonyl)amino) hexanoic acid (7). TLC system: 10% MeOH:DCM Rf: 0.3 LCMS (ESI): m/z 427.2 [M+H]⁺

### Methyl (2S)-2-((2S)-2-(((2-(3-chlorophenyl)-2,2-difluoro-1-(pyridin-3-yl)ethoxy)carbonyl)amino)hexanamido)-3-((S)-2-oxopyrrolidin-3-yl)propanoate (8)

To a stirred solution of (2S)-2-(((2-(3-chlorophenyl)-2,2-difluoro-1-(pyridin-3-yl)ethoxy) carbonyl)amino)hexanoic acid (7) (1.6 g, 3.75 mmol) in DMF (16 mL) was added, EDC·HCl (1.081 g, 5.532 mmol), HOBt (0.760 g, 5.632 mmol), DIPEA (1.9 mL, 11.12 mmol) and methyl (S)-2-amino-3-((S)-2-oxopyrrolidin-3-yl)propanoate (Amine fragment-2) (1.37 g, 7.41 mmol) at 0° C. The reaction mixture was allowed to stir at room temperature for 16 h. The reaction mixture was quenched with ice water (30 mL) and extracted with ethyl acetate (2×50 mL). The combined organic layer was washed with brine (50 mL), dried over sodium sulfate and evaporated under reduced pressure. The material was purified by combi-flash (C-18) to afford methyl (2S)-2-((2S)-2-(((2-(3-chlorophenyl)-2,2-difluoro-1-(pyridin-3-yl)ethoxy)carbonyl)amino)hexanamido)-3-((S)-2-oxopyrrolidin-3-yl)propanoate (8). TLC system: 10% Methanol in dichloromethane Rf: 0.5 LCMS (ESI): m/z 595.3 [M+H]⁺

### 2-(3-Chlorophenyl)-2,2-difluoro-1-(pyridin-3-yl)ethyl ((S)-1-(((S)-1-hydroxy-3-((S)-2-oxopyrrolidin-3-yl)propan-2-yl)amino)-1-oxohexan-2-yl)carbamate (9)

To a stirred solution of methyl (2S)-2-((2S)-2-(((2-(3-chlorophenyl)-2,2-difluoro-1-(pyridin-3-yl)ethoxy)carbonyl)amino)hexanamido)-3-((S)-2-oxopyrrolidin-3-yl)propanoate (8) (900 mg, 1.512 mmol) in MeOH (10 mL) was added NaBH₄ (862 mg, 22.689 mmol) at 0° C. and the reaction mixture stirred for 3 h at RT. The progress of the reaction was monitored by TLC. The reaction mixture was quenched with sat. Ammonium chloride solution (20 mL) and extracted with DCM (2×20 mL). The combined organic layer was washed with brine (20 mL), dried over Na₂SO₄ and concentrated to afford product, which was triturated with diethyl ether (10 mL) to afford 2-(3-chlorophenyl)-2,2-difluoro-1-(pyridin-3-yl)ethyl ((S)-1-(((S)-1-hydroxy-3-((S)-2-oxopyrrolidin-3-yl)propan-2-yl)amino)-1-oxohexan-2-yl)carbamate (9). TLC system: 10% Methanol in DCM Rf: 0.3 LCMS (ESI): m/z 567.23 [M+H]⁺

### 2-(3-Chlorophenyl)-2,2-difluoro-1-(pyridin-3-yl)ethyl ((S)-1-oxo-1-(((S)-1-oxo-3-((S)-2-oxopyrrolidin-3-yl)propan-2-yl)amino)hexan-2-yl)carbamate (A243)

To a stirred solution of 2-(3-chlorophenyl)-2,2-difluoro-1-(pyridin-3-yl)ethyl ((S)-1-(((S)-1-hydroxy-3-((S)-2-oxopyrrolidin-3-yl)propan-2-yl)amino)-1-oxohexan-2-yl)carbamate (9) (350 mg, 0.617 mmol) in ethyl acetate (10 mL) was added Dess-Martin periodinane (523 mg, 1.234 mmol) at 0° C. and stirred at RT for 3 h. After completion of the reaction by TLC, the reaction mixture was diluted with ethyl acetate (20 mL) and filtered through calcite pad and washed with satd. Hypo solution (3×20 mL) followed by sat. NaHCO₃ solution (3×20 mL). Organic layer was dried over anhydrous Na₂SO₄, filtered and concentrated to get a material which was purified by column chromatography combi-flash (RP, buffer: 0.1% ABC/ACN) to obtain 2-(3-cyclopropylphenyl)-2,2-difluoro-1-phenylethyl ((S)-4-methyl-1-oxo-1-(((S)-1-oxo-3-((S)-2-oxo pyrrolidin-3-yl) propan-2-yl)amino)pentan-2-yl)carbamate (A243). TLC system: 10% Methanol in DCM Rf: 0.4 LCMS (ESI): m/z 565.2 [M+H]⁺

### (3S)-3-((2S)-2-(((2-(3-Chlorophenyl)-2,2-difluoro-1-(pyridin-3-yl)ethoxy)carbonyl)amino)hexanamido)-1-(cyclopropylamino)-1-oxo-4-((S)-2-oxopyrrolidin-3-yl)butan-2-yl acetate (11)

To a stirred solution of 2-(3-chlorophenyl)-2,2-difluoro-1-(pyridin-3-yl)ethyl ((S)-1-oxo-1-(((S)-1-oxo-3-((S)-2-oxopyrrolidin-3-yl)propan-2-yl)amino)hexan-2-yl)carbamate (A243) (150 mg, 0.265 mmol) in DCM (3 mL) was added isocyanocyclopropane (10) (36 mg, 0.530 mmol) followed by acetic acid (0.2 mL, 0.530 mmol) at 0° C. and stirred at RT for 16 h. The progress of the reaction was monitored by TLC and LC-MS. The reaction mixture was diluted with dichloromethane (10 mL) and washed with 1N HCl (2×20 mL) followed by brine (1×20 mL). The organic layer was dried over anhydrous Na₂SO₄ and evaporated under reduced pressure to afford (3S)-3-((2S)-2-(((2-(3-chlorophenyl)-2,2-difluoro-1-(pyridin-3-yl)ethoxy) carbonyl)amino)hexanamido)-1-(cyclopropylamino)-1-oxo-4-((S)-2-oxopyrrolidin-3-yl)butan-2-yl acetate (11). TLC system: 10% Methanol in dichloromethane Rf: 0.5 LCMS (ESI): m/z 692.33 [M+H]⁺

### 2-(3-Chlorophenyl)-2,2-difluoro-1-(pyridin-3-yl)ethyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (12)

To a stirred solution of (3S)-3-((2S)-2-(((2-(3-chlorophenyl)-2,2-difluoro-1-(pyridin-3-yl)ethoxy)carbonyl)amino)hexanamido)-1-(cyclopropylamino)-1-oxo-4-((S)-2-oxopyrrolidin-3-yl)butan-2-yl acetate (11) (150 mg, 0.216 mmol) in MeOH (4.5 mL) was added K₂CO₃ (60 mg, 0.433 mmol) at RT and stirred at room temperature for 3 h. The progress of the reaction was monitored by TLC. The reaction mixture was completely distilled under reduced pressure and diluted with water (10 mL) and extracted with ethyl acetate (2×25 mL), dried over sodium sulfate, concentrated under reduced pressure to afford 2-(3-chlorophenyl)-2,2-difluoro-1-(pyridin-3-yl)ethyl((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (12). TLC system: 10% Methanol in dichloromethane Rf: 0.5 LCMS (ESI): m/z 650.7 [M+H]

### 2-(3-chlorophenyl)-2,2-difluoro-1-(pyridin-3-yl)ethyl ((S)-1-(((S)-4-(cyclopropylamino)-3,4-dioxo-1-((S)-2-oxopyrrolidin-3-yl)butan-2-yl)amino)-1-oxohexan-2-yl)carbamate (A243A)

To a stirred solution of 2-(3-chlorophenyl)-2,2-difluoro-1-(pyridin-3-yl)ethyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (12) (100 mg, 0.153 mmol) in ethyl acetate (5 mL) was added Dess-Martin periodinane (130 mg, 0.307 mmol) at 0° C. and stirred at RT for 3 h. The progress of the reaction was monitored by TLC and LC-MS. The suspension was filtered through diatomaceous earth pad and washed with ethyl acetate (20 mL) and filtrate was washed with hypo solution (3×20 mL) followed by saturated NaHCO₃ solution (3×20 mL). Organic layer was dried over anhydrous Na₂SO₄, filtered and concentrated to get a material, which was purified by combi-flash (C-18, 0.1% ABC/ACN) to afford 2-(3-chlorophenyl)-2,2-difluoro-1-(pyridin-3-yl)ethyl ((S)-1-(((S)-4-(cyclopropylamino)-3,4-dioxo-1-((S)-2-oxopyrrolidin-3-yl)butan-2-yl)amino)-1-oxohexan-2-yl)carbamate (A243A). TLC system: 10% Methanol in dichloromethane Rf: 0.4 LCMS (ESI): m/z 648.2 [M+H]⁺

### Example 32: Synthesis of Compound A244

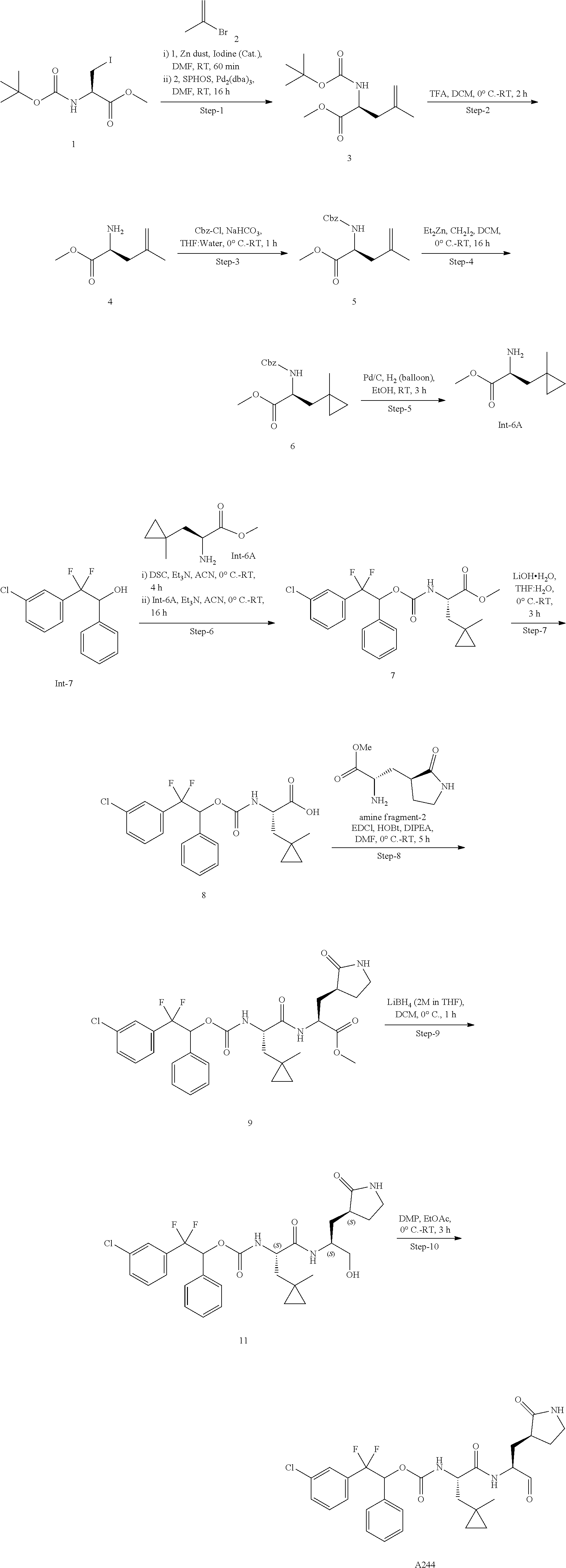

### methyl (S)-2-((tert-butoxycarbonyl)amino)-4-methylpent-4-enoate (3)

To a suspension of activated zinc (6.07 mg, 3.03 mmol) in DMF (50 ml) was added catalytic amount of Iodine at RT and stirred for 10 min and then added methyl (R)-2-((tert-butoxy carbonyl) amino)-3-iodopropanoate (1) (1 g, 3.03 mmol) portion wise at same temperature followed by addition of catalytic amount of iodine and stirred for 60 min at same temperature. Reaction mass degassed with argon balloon for 15 min. and added 2-bromoprop-1-ene (2) (361 mg, 3.39 mmol), Pd₂(dppf)Cl₂ (55 mg, 0.060 mmol) and SPHOS (24 mg, 0.060 mmol) at RT and heated to 50° C. for 12 h. The progress of the reaction was monitored by TLC and LCMS. After completion of starting material, reaction mixture was filtered through diatomaceous earth bed and bed was washed with ethyl acetate. Obtained filtrate was diluted with water (50 mL) and extracted with ethyl acetate (2×30 mL). Combined organic layer was washed with brine solution (50 mL), dried over anhydrous sodium sulfate and evaporated under reduced pressure. The residue was purified by column chromatography using silica gel (100-200 mesh) and 10% EtOAc:Pet.Ether as eluent to afford methyl (S)-2-((tert-butoxycarbonyl)amino)-4-methylpent-4-enoate (3). TLC system: 10% Ethyl acetate in hexane Rf: 0.6 methyl (S)-2-amino-4-methylpent-4-enoate (4):

To a stirred solution of (S)-2-((tert-butoxycarbonyl)amino)-4-methylpent-4-enoate (4) (7.0 g, 28.6 mmol) in DCM (70 mL) was added TFA (20 mL) at 0° C. and stirred at RT for 2 h. The progress of the reaction was monitored by TLC. After completion of starting material, reaction mixture was concentrated under reduced pressure to afford methyl (S)-2-amino-4-methylpent-4-enoate (4). LCMS (ESI): m/z 144.42 [M+H]⁺ TLC system: 20% Ethyl acetate in hexane Rf: 0.1

### methyl(S)-2-(((benzyloxy)carbonyl)amino)-4-methylpent-4-enoate (5)

To a stirred solution of methyl (S)-2-amino-4-methylpent-4-enoate (4) (500 mg, 3.4 mmol) in THE (8 mL) and water (2 ml) was added NaHCO₃ (720 mg, 8.68 mmol) at RT and Cbz-Cl (880 mg, 5.2 mmol) at 0° C. and stirred at RT for 1 h. The progress of the reaction was monitored by TLC and LCMS. After completion of starting material, reaction mixture was diluted with ice cold water (20 mL) and extracted with EtOAc (2×20 mL). Combined organic layer was washed with brine solution (30 mL), dried over anhy. Na₂SO₄ and evaporated under reduced pressure. Obtained material was purified by normal phase chromatography, using silica gel (100-200 mesh) and 15% EtOAc/Pet.ether as eluent to afford methyl methyl (S)-2-(((benzyloxy) carbonyl)amino)-4-methylpent-4-enoate (5). TLC system: 10% Ethyl acetate in hexane Rf: 0.5 LCMS (ESI): m/z 278.1 [M+H]⁺

### methyl(S)-2-(((benzyloxy)carbonyl)amino)-3-(1-methylcyclopropyl)propanoate (6)

To a stirred solution of methyl (S)-2-(((benzyloxy)carbonyl)amino)-4-methylpent-4-enoate (5) (10 g, 36.1 mmol) in DCM (100 mL) was added Et₂Zn (1M in THF, 144 mL, 144.4 mmol) slowly drop wise at 0° C. and stirred at 0° C. for 10 min. After that added CH₂I₂ (2.8 g, 108.3 mmol) and reaction was stirred at RT for 16 h. After completion of the reaction (monitored by TLC), the reaction mixture was quenched with 1N HCl solution (30 mL) at 0° C. and was extracted with ethyl acetate (2×100 mL). Combined organic layer was washed with water (100 mL), dried over anhy. Na₂SO₄ and concentrated under reduced pressure. Obtained residue was purified by column chromatography using silica gel (100-200 mesh) and 20% ethyl acetate as eluent to afford methyl (S)-2-(((benzyloxy)carbonyl)amino)-3-(1-methylcyclopropyl)propanoate (6). TLC system: 10% Ethyl acetate Rf: 0.3 LCMS (ESI): m/z 292.14 [M+H]⁺

### methyl (S)-2-amino-3-(1-methylcyclopropyl) propionate (Int-6A)

To a degassed solution of methyl (S)-2-(((benzyloxy)carbonyl)amino)-3-(1-methylcyclopropyl)propanoate (6) (4 g, 13.7 mmol) in EtOH (40 mL) was added Pd/C (2 g, 50% w/w) at RT and stirred for 3 h under H₂ balloon pressure. Reaction progress was monitored by TLC. After completion of starting material, reaction mass was filtered through diatomaceous earth pad and bed was washed with EtOH (30 mL). Obtained filtrate was concentrated under reduced pressure to afford methyl (S)-2-amino-3-(1-methylcyclopropyl)propanoate (Int-6) TLC system: 20% Ethyl acetate in hexane Rf: 0.1 LCMS (ESI): m/z 158.1 [M+H]⁺

### methyl(2S)-2-(((2-(3-chlorophenyl)-2,2-difluoro-1-phenylethoxy)carbonyl)amino)-3-(1-methylcyclopropyl)propanoate (7)

To a stirred solution of 2-(3-chlorophenyl)-2,2-difluoro-1-phenylethan-1-ol (common Int-7) (6.0 g, 22.3 mmol) in ACN (40 mL) was added N, N′-disuccinamidyl carbonate (8.59 g, 33.5 mmol) followed by triethylamine (12 mL, 89.5 mmol) at 0° C. and stirred the reaction mixture at room temperature for 2 h. The progress of the reaction was monitored by TLC. The reaction mass was used directly in the subsequent reaction.

In another RB flask, methyl (S)-2-amino-3-(1-methylcyclopropyl)propanoate (Int-6A) (int-5) in ACN (40 mL) was treated with triethylamine (12 ml, 89.5 mmol). The resulting reaction mixture was stirred for 5 min, then added above prepared reaction mass drop-wise and the reaction mixture was stirred at room temperature for 16 h. After completion of the reaction by TLC, reaction mixture was quenched with ice water (200 mL) and extracted with ethyl acetate (2×100 mL). Combined organic layer and washed with water (100 mL), dried over anhy. Na₂SO₄ and concentrated under reduced pressure. Obtained residue was purified by column chromatography using silica gel (100-200 mesh) and 20% ethyl acetate as eluent to afford methyl (2S)-2-(((2-(3-chlorophenyl)-2,2-difluoro-1-phenylethoxy)carbonyl)amino)-3-(1-methylcyclopropyl)propanoate (7). TLC system: 10% Ethyl acetate in hexane Rf: 0.55 LCMS (ESI): m/z 452.20 [M+H]⁺

### ((2S)-2-(((2-(3-chlorophenyl)-2,2-difluoro-1-phenylethoxy)carbonyl)amino)-3-(1-methylcyclopropyl)propanoic acid (8)

To a stirred solution of methyl (2S)-2-(((2-(3-chlorophenyl)-2,2-difluoro-1-phenylethoxy) carbonyl)amino)-3-(1-methylcyclopropyl)propanoate (7) (3 g, 6.65 mmol) in THE (14 mL) and water (7 mL) was added LiOH·H₂O (558 mg, 13.3 mmol) at 0° C. and stirred at room temperature for 3 h. The progress of the reaction was monitored by TLC. THE was distilled under reduced pressure and compound was acidified with aq. 1N HCl solution up to pH˜2 and extracted with DCM (2×200 mL). Combined organic layer was washed with water (100 mL), brine solution (150 mL), dried over any. sodium sulfate and concentrated under reduced pressure to afford (2S)-2-(((2-(3-chlorophenyl)-2,2-difluoro-1-phenylethoxy)carbonyl)amino)-3-(1-methylcyclopropyl) propanoic acid (8). TLC system: 10% Methanol in dichloromethane Rf: 0.1 LCMS (ESI): m/z 438.2 [M+H]⁺

### methyl (2S)-2-((2S)-2-(((2-(3-chlorophenyl)-2,2-difluoro-1-phenylethoxy)carbonyl)amino)-3-(1-methylcyclopropyl)propanamido)-3-((S)-2-oxopyrrolidin-3-yl)propanoate (9)

To a stirred solution of (2S)-2-(((2-(3-chlorophenyl)-2,2-difluoro-1-phenylethoxy) carbonyl) amino)-3-(1-methylcyclopropyl) propanoic acid (8) (3 g, 6.86 mmol) in DMF (30 mL) was added EDC·HCl (1.5 g, 8.2 mmol), HOBt (1.0 g, 8.2 mmol), DIPEA (3.6 mL, 20.5 mmol) and methyl (S)-2-amino-3-((R)-2-oxopyrrolidin-3-yl) propanoate (Amine fragment-2) (2.2 g, 12.3 mmol) simultaneously at 0° C. and stirred at room temperature for 5 h. After completion of the reaction (monitored by TLC), the reaction mixture was quenched with ice water (30 mL) and extracted with ethyl acetate (2×80 mL). Combined the organic layer and washed with brine solution (2×50 mL), dried over sodium sulfate and evaporated under reduced pressure. The residue was purified by combi-flash NP, compound eluted at 40% ethyl acetate to afford methyl (2S)-2-((2S)-2-(((2-(3-chlorophenyl)-2,2-difluoro-1-phenylethoxy) carbonyl)amino)-3-(1-methylcyclopropyl)propanamido)-3-((S)-2-oxopyrrolidin-3-yl)propanoate (9). TLC system: 100% Ethyl acetate Rf: 0.4 LCMS (ESI): m/z 606.4 [M+H]⁺

### 2-(3-chlorophenyl)-2,2-difluoro-1-phenylethyl ((S)-1-(((S)-1-hydroxy-3-((S)-2-oxopyrrolidin-3-yl)propan-2-yl)amino)-3-(1-methylcyclopropyl)-1-oxopropan-2-yl)carbamate (10)

To a solution of afford methyl (2S)-2-((2S)-2-(((2-(3-chlorophenyl)-2,2-difluoro-1-phenylethoxy) carbonyl) amino)-3-(1-methylcyclopropyl) propanamido)-3-((S)-2-oxopyrrolidin-3-yl)propanoate (9) (2.0 g, 3.30 mmol) in THF (40 mL) was added LiBH₄ (2M in THF, 3.3 mL, 6.6 mmol) slowly drop wise at 0° C. and stirred for 1 h at 0° C. The progress of the reaction was monitored by TLC. After completion of starting material, reaction mixture was quenched with saturated NH₄Cl solution and extracted with dichloromethane (2×100 mL). Combined organic layer was washed with water, dried over anhydrous sodium sulfate and concentrated under reduced pressure. Obtained material was purified by reverse phase column (C18) eluting with 50% ACN in 0.1% FA in water to afford 2-(3-chlorophenyl)-2,2-difluoro-1-phenylethyl ((S)-1-(((S)-1-hydroxy-3-((S)-2-oxopyrrolidin-3-yl)propan-2-yl)amino)-3-(1-methylcyclopropyl)-1-oxopropan-2-yl)carbamate (10). TLC system: 5% Methanol in dichloromethane Rf: 0.35 LCMS (ESI): m/z 578.43 [M+H]⁺

### 2-(3-chlorophenyl)-2,2-difluoro-1-phenylethyl ((S)-3-(1-methylcyclopropyl)-1-oxo-1-(((S)-1-oxo-3-((S)-2-oxopyrrolidin-3-yl)propan-2-yl)amino)propan-2-yl)carbamate (A244)

To a stirred solution of 2-(3-chlorophenyl)-2,2-difluoro-1-phenylethyl ((S)-1-(((S)-1-hydroxy-3-((S)-2-oxopyrrolidin-3-yl)propan-2-yl)amino)-3-(1-methylcyclopropyl)-1-oxopropan-2-yl)carbamate (10) (700 mg, 1.21 mmol) in ethyl acetate (20 mL) was added Dess-Martin period inane (1.02 g, 2.42 mmol) slowly portion wise at 0° C. and stirred at RT for 2 h. The progress of the reaction was monitored by TLC and LCMS. Reaction mixture was diluted with Ethyl acetate (50 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure. Obtained material was purified by reverse phase combiflash column (C18) using 50% ACN in 0.1% aq. NH₄CO₃ solution as eluent to afford 2-(3-chlorophenyl)-2,2-difluoro-1-phenylethyl ((S)-3-(1-methylcyclopropyl)-1-oxo-1-(((S)-1-oxo-3-((S)-2-oxopyrrolidin-3-yl)propan-2-yl)amino)propan-2-yl)carbamate (A244). TLC system: 5% Methanol in DCM R_{f}: 0.55 LCMS (ESI): m/z 576.3 [M+H]⁺

### Example 33: Synthesis of Compound A245

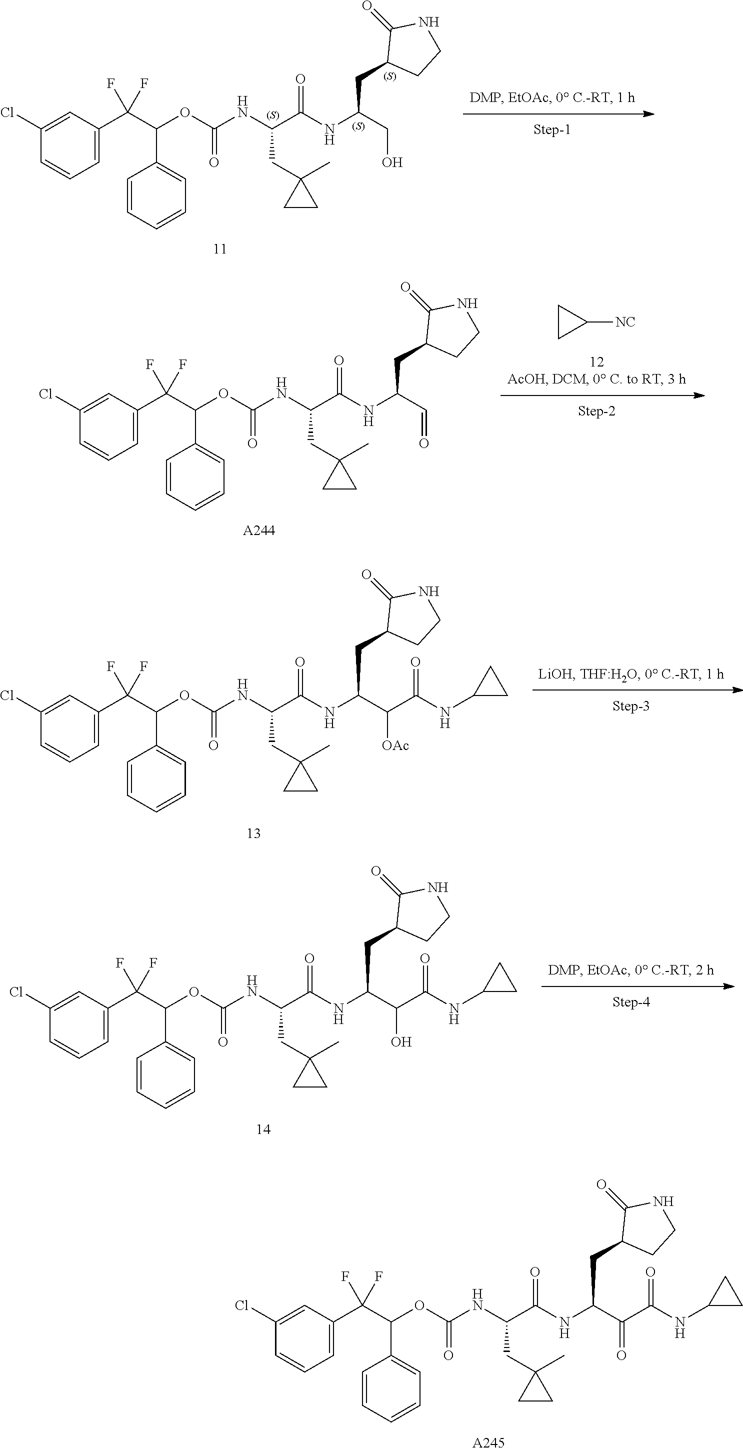

### 2-(3-chlorophenyl)-2,2-difluoro-1-phenyl ethyl ((S)-3-(1-methylcyclopropyl)-1-oxo-1-(((S)-1-oxo-3-((S)-2-oxopyrrolidin-3-yl)propan-2-yl)amino)propan-2-yl)carbamate (A244)

To a stirred solution of 2-(3-chlorophenyl)-2,2-difluoro-1-phenylethyl ((S)-1-(((S)-1-hydroxy-3-((S)-2-oxopyrrolidin-3-yl)propan-2-yl)amino)-3-(1-methylcyclopropyl)-1-oxopropan-2-yl)carbamate (11) (700 mg, 1.21 mmol) in ethyl acetate (20 mL) was added Dess-Martin period inane (1.02 g, 2.42 mmol) slowly portion wise at 0° C. and stirred at RT for 1 h. The progress of the reaction was monitored by TLC and LCMS. Reaction mixture was diluted with Ethyl acetate (50 mL) and filtered through diatomaceous earth pad. Obtained filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure to afford 2-(3-chlorophenyl)-2,2-difluoro-1-phenylethyl ((S)-3-(1-methylcyclo propyl)-1-oxo-1-(((S)-1-oxo-3-((S)-2-oxopyrrolidin-3-yl)propan-2-yl)amino) propan-2-yl)carbamate (A244). TLC system: 10% Methanol in DCM R_{f}: 0.55 LCMS (ESI): m/z 576.2 [M+H]⁺

### (3S)-3-((2S)-2-(((2-(3-chlorophenyl)-2,2-difluoro-1-phenylethoxy)carbonyl)amino)-3-(1-methylcyclopropyl)propanamido)-1-(cyclopropylamino)-1-oxo-4-((S)-2-oxopyrrolidin-3-yl)butan-2-yl acetate (13)

To a stirred solution of 2-(3-chlorophenyl)-2,2-difluoro-1-phenylethyl ((S)-3-(1-methylcyclopropyl)-1-oxo-1-(((S)-1-oxo-3-((S)-2-oxopyrrolidin-3-yl)propan-2-yl)amino)propan-2-yl) carbamate (A244) (700 mg, 1.21 mmol) in dichloromethane (7 mL) was added acetic acid (0.6 ml) and cyclopropyl isocyanide (12) (687 mg, 10.25 mmol) at 0° C. and stirred at RT for 3 h. Reaction was monitored by TLC and LCMS. After completion of the reaction, reaction mixture was diluted with DCM (20 mL) and washed with water (3×40 mL), brine solution. Organic layer was separated, dried over anhy. Na₂SO₄, filtered and concentrated under reduced pressure. Obtained material was purified by reverse phase comb flash column (C18) using 50% ACN in 0.1% aq. NH₄CO₃ solution as eluent to afford (3S)-3-((2S)-2-(((2-(3-chlorophenyl)-2,2-difluoro-1-phenylethoxy) carbonyl) amino)-3-(1-methylcyclo propyl) propanamido)-1-(cyclopropylamino)-1-oxo-4-((S)-2-oxopyrrolidin-3-yl)butan-2-yl acetate (13). TLC system: 10% Methanol in dichloromethane R_{f}: 0.4 LCMS (ESI): m/z 703.3 (M+H)⁺

### 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-3-(1-methylcyclopropyl)-1-oxopropan-2-yl) carbamate (14)

To a stirred solution of (3S)-3-((2S)-2-(((2-(3-chlorophenyl)-2,2-difluoro-1-phenylethoxy) carbonyl) amino)-3-(1-methylcyclopropyl) propanamido)-1-(cyclopropylamino)-1-oxo-4-((S)-2-oxopyrrolidin-3-yl) butan-2-yl acetate (13) (400 mg, 0.56 mmol) in THE (4 mL), water (2 mL) was added LiOH·H₂O (47 mg, 1.13 mmol) at 0° C. and stirred at room temperature for 1 h. The progress of the reaction was monitored by TLC and LCMS. After completion of the reaction, reaction mixture was extracted with ethyl acetate (2×100 mL). Combined organic layer was dried over anhy. Na₂SO₄ and concentrated under reduced pressure to afford 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-3-(1-methylcyclopropyl)-1-oxo propan-2-yl)carbamate (14). TLC system: 10% Methanol in dichloromethane Rf: 0.3 LCMS (ESI): m/z 661.7 (M+H)⁺

### 2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-3-(1-methylcyclopropyl)-1-oxopropan-2-yl)carbamate (A245)

To a stirred solution of 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-3-(1-methylcyclopropyl)-1-oxo propan-2-yl)carbamate (14) (320 mg, 0.48 mmol) in Ethyl acetate (15 mL) was added Dess-Martin periodinane (411 mg, 1.13 mmol) at 0° C. and stirred at RT for 2 h. After completion of the reaction, reaction mixture was diluted with Ethyl acetate (20 mL) and filtered through diatomaceous earth pad. Obtained filtrate was washed with sat. Hypo solution (3×50 mL) followed by sat. NaHCO₃ solution (3×50 mL). Organic layer was separated, dried over anhydrous Na₂SO₄, filtered and concentrated under reduced pressure. Obtained compound was purified by trituration with n-Pentane/DEE to afford 2-(3-chlorophenyl)-2,2-difluoro-1-phenylethyl ((S)-1-(((S)-4-(cyclopropylamino)-3,4-dioxo-1-((S)-2-oxo pyrrolidin-3-yl)butan-2-yl)amino)-3-(1-methylcyclopropyl)-1-oxopropan-2-yl)carbamate (A245). TLC system: 5% Methanol in DCM Rf: 0.5 LCMS (ESI): m/z 659.3 [M+H]⁺

### Example 34: Synthesis of Compound A246

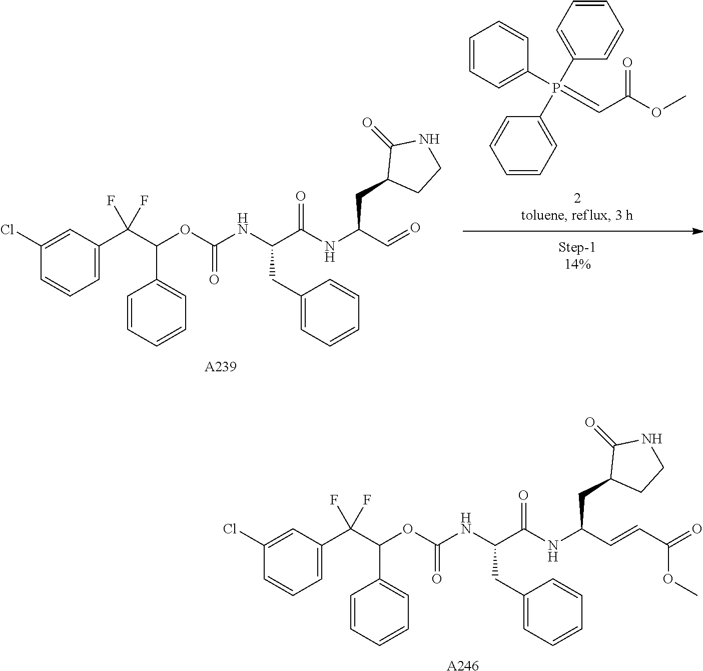 [SMILES: O=C[C@H](C[C@@H]1CCNC1=O)NC(=O)[C@H](Cc1ccccc1)NC(=O)OC(c1ccccc1)C(F)(F)c1cccc(Cl)c1 ; COC(=O)C=P(c1ccccc1)(c1ccccc1)c1ccccc1 ; "A239" ; COC(=O)/C=C/[C@H](C[C@@H]1CCNC1=O)NC(=O)[C@H](Cc1ccccc1)NC(=O)OC(c1ccccc1)C(F)(F)c1cccc(Cl)c1 ; "A246"]

### Methyl(4S,E)-4-((2S)-2-(((2-(3-chlorophenyl)-2,2-difluoro-1-phenylethoxy)carbonyl)amino)-3-phenylpropanamido)-5-((S)-2-oxopyrrolidin-3-yl)pent-2-enoate (A246)

To a stirred solution of 2-(3-chlorophenyl)-2,2-difluoro-1-phenylethyl ((S)-1-oxo-1-(((S)-1-oxo-3-((S)-2-oxopyrrolidin-3-yl)propan-2-yl)amino)-3-phenylpropan-2-yl)carbamate (A239) (100 mg, 0.1672 mmol) in Toluene (5 mL) was added methyl 2-(triphenyl-phosphaneylidene)acetate (2) (55.8 mg, 0.1672 mmol) and refluxed for 3 h. The progress of the reaction was monitored by TLC and LC-MS. The reaction mixture was concentrated and purified by prep. HPLC to afford methyl(4S,E)-4-((2S)-2-(((2-(3-chlorophenyl)-2,2-difluoro-1-phenylethoxy)carbonyl)amino)-3-phenyl propanamido)-5-((S)-2-oxopyrrolidin-3-yl)pent-2-enoate (A246). TLC system: 50% EtOAc in Petroleum ether Rf: 0.5 LCMS (ESI): m/z 654.3 [M+H]⁺

### Example 35: Synthesis of Compounds A247 and A248

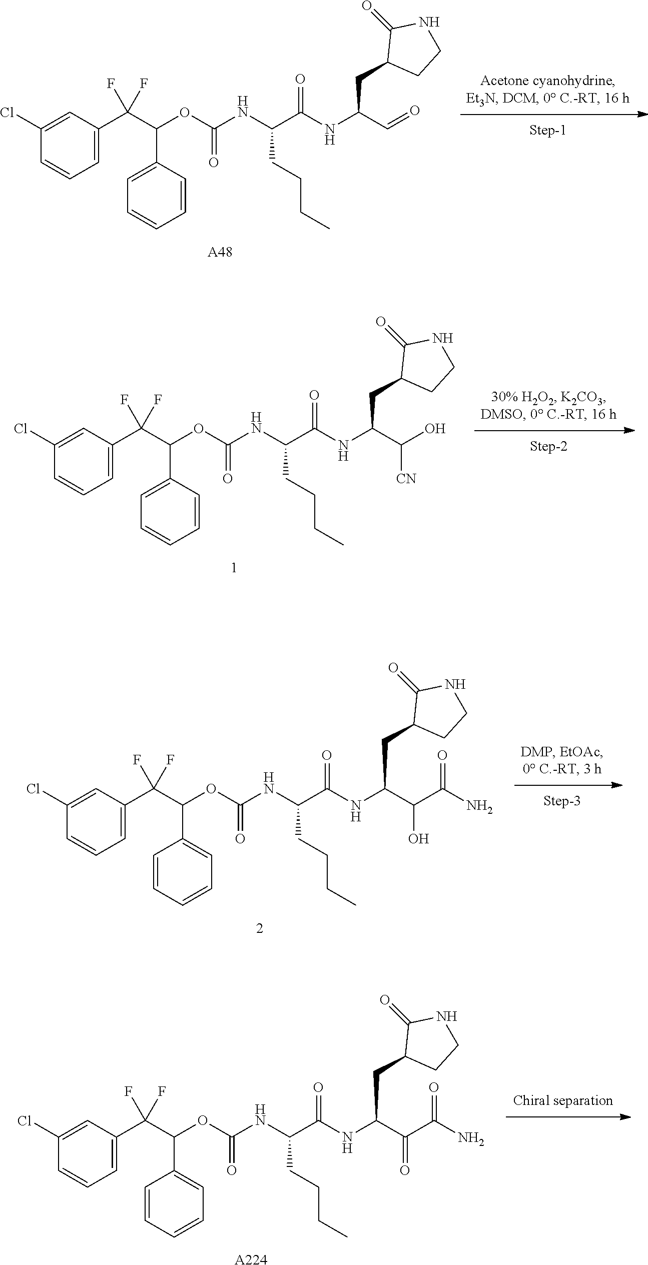

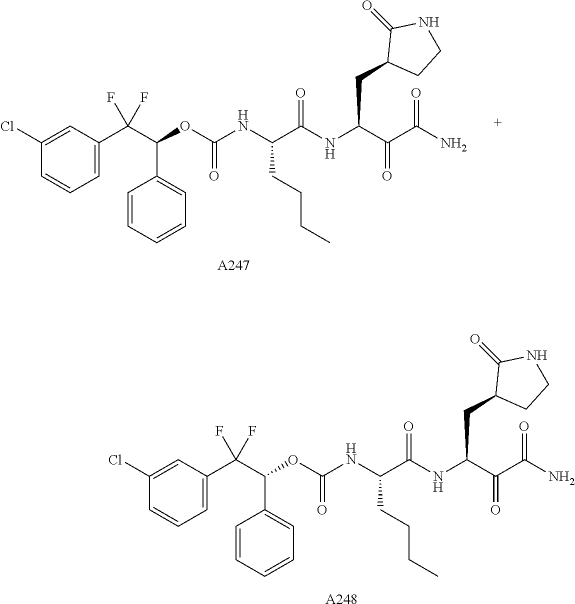 [SMILES: CCCC[C@H](NC(=O)O[C@@H](c1ccccc1)C(F)(F)c1cccc(Cl)c1)C(=O)N[C@@H](C[C@@H]1CCNC1=O)C(=O)C(N)=O ; "A247" ; CCCC[C@H](NC(=O)O[C@H](c1ccccc1)C(F)(F)c1cccc(Cl)c1)C(=O)N[C@@H](C[C@@H]1CCNC1=O)C(=O)C(N)=O ; "A248"]

### 2-(3-Chlorophenyl)-2,2-difluoro-1-phenylethyl ((2S)-1-(((2S)-1-cyano-1-hydroxy-3-((S)-2-oxopyrrolidin-3-yl) propan-2-yl) amino)-1-oxohexan-2-yl) carbamate (1)

To a stirred solution of 2-(3-chlorophenyl)-2,2-difluoro-1-phenylethyl ((S)-1-oxo-1-(((S)-1-oxo-3-((S)-2-oxopyrrolidin-3-yl) propan-2-yl) amino) hexan-2-yl) carbamate (A48) (500 mg, 0.886 mmol) in DCM (10 mL) was added acetonecyanohydrine (0.5 mL, 1 vol), Et₃N (0.5 mL, 1 vol) at 0° C. and stirred at RT for 16 h. The progress of the reaction was monitored by TLC. The reaction mixture was diluted with DCM (15 mL) and washed with water (2×15 mL), dried over sodium sulfate and evaporated under reduced pressure to get the product. This material was triturated with n-pentane (25 mL) to afford 2-(3-chlorophenyl)-2,2-difluoro-1-phenylethyl ((2S)-1-(((2S)-1-cyano-1-hydroxy-3-((S)-2-oxopyrrolidin-3-yl) propan-2-yl) amino)-1-oxohexan-2-yl) carbamate (1). TLC system: 10% Methanol in dichloromethane Rf: 0.5 LCMS (ESI): m/z 591.3 [M+H]⁺

### 2-(3-Chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-1-oxohexan-2-yl) carbamate (2)

To a stirred solution of 2-(3-chlorophenyl)-2,2-difluoro-1-phenylethyl ((2S)-1-(((2S)-1-cyano-1-hydroxy-3-((S)-2-oxopyrrolidin-3-yl) propan-2-yl) amino)-1-oxohexan-2-yl) carbamate (1) (1 g, 0.8474 mmol) in DMSO (5 mL) was added K₂CO₃ (350 mg, 2.54 mmol) followed by 30% H₂O₂ (4 mL, 4 vol) at 0° C. and stirred at RT for 16 h. The progress of the reaction was monitored by TLC. The reaction mixture was quenched with Sat. ammonium chloride solution (20 mL) and extracted with ethyl acetate (2×50 mL). The combined organic layer was washed with water (2×15 mL) followed by brine (1×15 mL), dried over sodium sulfate and evaporated under reduced pressure to get the material, which was triturated with n-pentane (15 mL) to afford 2-(3-chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-1-oxohexan-2-yl) carbamate (2). TLC system: 10% Methanol in dichloromethane Rf: 0.3 LCMS (ESI): m/z 609.39 [M+H]⁺

### 2-(3-Chlorophenyl)-2,2-difluoro-1-phenylethyl ((S)-1-(((S)-4-amino-3,4-dioxo-1-((S)-2-oxopyrrolidin-3-yl) butan-2-yl) amino)-1-oxohexan-2-yl) carbamate (A224)

To a stirred solution of 2-(3-chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-1-oxohexan-2-yl) carbamate (2) (640 mg, 1.050 mmol) in ethyl acetate (10 mL) was added Dess-Martin periodinane (1.113 g, 2.627 mmol) at 0° C. and stirred at RT for 3 h. The progress of the reaction was monitored by TLC and LC-MS. The reaction mixture was filtered through diatomaceous earth bed and washed with ethyl acetate (20 mL). Filtrate was washed with sat. Hypo solution (3×20 mL), sat. NaHCO₃ solution (3×20 mL) followed by brine (1×15 mL). Organic layer was dried over anhydrous Na₂SO₄, filtered and concentrated to get the material, which was purified by reverse phase chromatography by using 0.1% ammonium carbonate in water/acetonitrile as buffer to afford 2-(3-chlorophenyl)-2,2-difluoro-1-phenylethyl ((S)-1-(((S)-4-amino-3,4-dioxo-1-((S)-2-oxo pyrolidin-3-yl) butan-2-yl) amino)-1-oxohexan-2-yl) carbamate (A224). TLC system: 10% Methanol in dichloromethane Rf: 0.4 LCMS (ESI): m/z 607.2 [M+H]⁺

SFC Purification; the obtained racemate (500 mg) was purified by Chiral SFC to get two isomers. (S)-2-(3-chlorophenyl)-2,2-difluoro-1-phenylethyl((S)-1-(((S)-4-amino-3,4-dioxo-1-((S)-2-oxopyrrolidin-3-yl)butan-2-yl) amino)-1-oxohexan-2-yl) carbamate (A247). (R)-2-(3-chlorophenyl)-2,2-difluoro-1-phenylethyl ((S)-1-(((S)-4-amino-3,4-dioxo-1-((S)-2-oxopyrrolidin-3-yl) butan-2-yl) amino)-1-oxohexan-2-yl)carbamate (A248), A247; LCMS (ESI): m/z 607.3 [M+H]⁺, A248; LCMS (ESI): m/z 607.3 [M+H]⁺

### Example 36: Synthesis of Compound A249

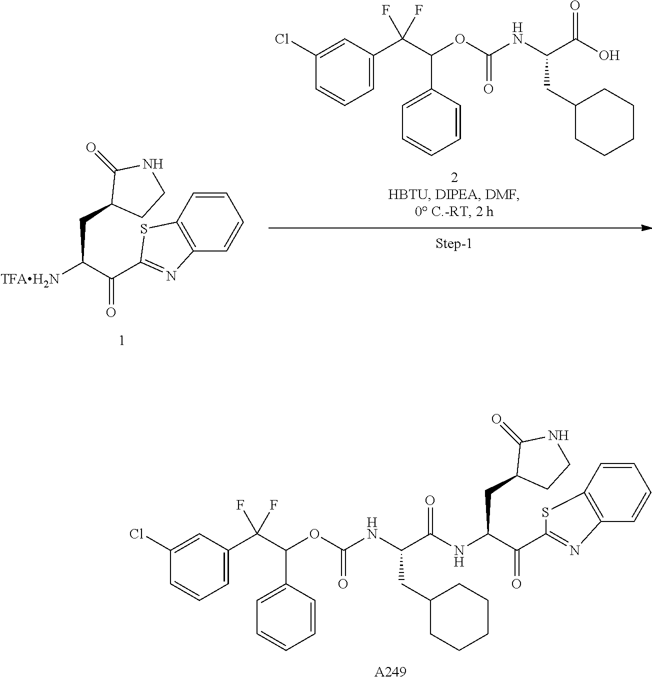

### 2-(3-chlorophenyl)-2,2-difluoro-1-phenylethyl ((S)-1-(((S)-1-(benzo[d]thiazol-2-yl)-1-oxo-3-((S)-2-oxopyrrolidin-3-yl)propan-2-yl)amino)-3-cyclohexyl-1-oxopropan-2-yl)carbamate (A249)

To a stirred solution of (S)-3-((S)-2-amino-3-(benzo[d]thiazol-2-yl)-3-oxopropyl)pyrrolidin-2-one as TFA salt (1) (0.30 g, 0.77 mmol) in DMF (5 mL) was added (2S)-2-(((2-(3-chlorophenyl)-2,2-difluoro-1-phenylethoxy)carbonyl)amino)-3-cyclohexylpropanoic acid (2) (0.36 g, 0.77 mmol), HBTU (0.29 g, 0.77 mmol) and DIPEA (0.42 mL, 2.33 mmol) at 0° C. The resulting reaction mixture was stirred at RT for 2 h. After completion of the reaction (monitored by TLC and LC-MS), the reaction mixture was diluted with water (50 mL) and extracted with EtOAc (2×25 mL). The combined organic layer was washed with brine solution (50 mL), dried over anhy. Na₂SO₄ and concentrated under reduced pressure. Obtained material was purified by prep HPLC to afford 2-(3-chlorophenyl)-2,2-difluoro-1-phenylethyl ((S)-1-(((S)-1-(benzo[d]thiazol-2-yl)-1-oxo-3-((S)-2-oxopyrrolidin-3-yl)propan-2-yl)amino)-3-cyclohexyl-1-oxopropan-2-yl)carbamate (A249). TLC system: 80% Ethyl acetate in pet ether Rf: 0.2 LCMS (ESI): m/z 737.2 [M+H]⁺

### Example 37: Synthesis of Compound A250

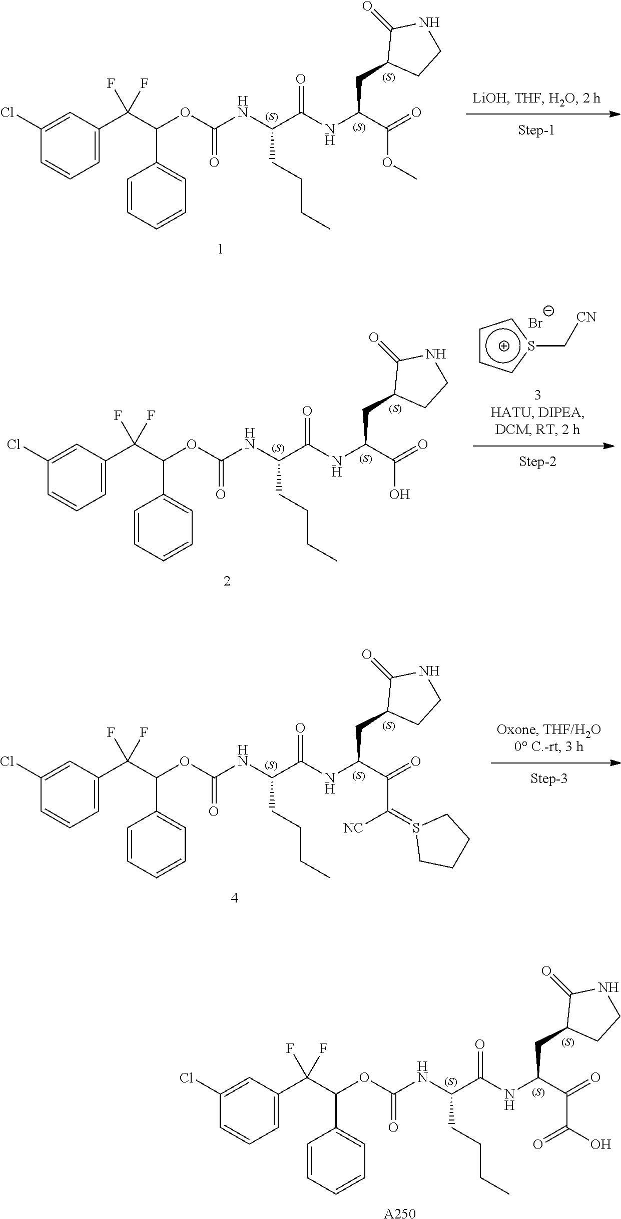

### (2S)-2-((2S)-2-(((2-(3-chlorophenyl)-2,2-difluoro-1-phenylethoxy) carbonyl) amino) hexanamido)-3-((S)-2-oxopyrrolidin-3-yl)propanoic acid (2)

To a stirred solution of methyl (2S)-2-((2S)-2-(((2-(3-chlorophenyl)-2,2-difluoro-1-phenylethoxy) carbonyl) amino)hexanamido)-3-((S)-2-oxopyrrolidin-3-yl)propanoate (1) (5.5 g, 9.25 mmol) in THE (30 mL) water (30 mL) was added LiOH·H₂O (569 mg, 13.88 mmol) at 0° C. and stirred at RT for 2 h. The progress of the reaction was monitored by TLC. The reaction mixture was acidified with w N HCl (20 mL) and extracted with Ethyl acetate (2×100 mL. The combined organic layer was washed with water (2×50 mQ, dried over sodium sulfate and evaporated under reduced pressure to get the product. This material was triturated with n-pentane (50 mL) to afford (2S)-2-((2S)-2-(((2-(3-chlorophenyl)-2,2-difluoro-1-phenylethoxy) carbonyl) amino) hexanamido)-3-((S)-2-oxopyrrolidin-3-yl) propanoic acid (2). TLC system: 10% Methanol in dichloromethane Rf: 0.3 LCMS (ESI): m/z 580.2 [M+H]⁺

### 2-(3-chlorophenyl)-2,2-difluoro-1-phenylethyl ((S)-1-(((S)-4-cyano-3-oxo-1-((S)-2-oxopyrrolidin-3-yl)-4-(tetrahydro-1A4-thiophen-1-ylidene)butan-2-yl)amino)-1-oxohexan-2-yl)carbamate (4)

To a stirred solution of (2S)-2-((2S)-2-(((2-(3-chlorophenyl)-2,2-difluoro-1-phenyl ethoxy)carbonyl) amino) hexanamido)-3-((S)-2-oxopyrrolidin-3-yl)propanoic acid (2) (4.8 g, 8.275 mmol) in DCM (15 mL) was added HATU (4.7 g 12.41 mmol), 1-(cyanomethyl) tetrahydro-1H-thiophen-1-ium bromide (3) (2 g, 9.93 mmol), DIPEA (4.3 mL, 24.82 mmol) at 0° C. and stirred at RT for 3 h. The progress of the reaction was monitored by TLC and LC-MS. Reaction mixture was diluted with DCM (50 mL) and washed with sat NaHCO₃ solution (3×40 mL) followed by brine (1×20 mL). Organic layer was dried over anhydrous Na₂SO₄, filtered and concentrated to get material that was purified by normal phase chromatography by using ethylacetate in petroleum ether to afford 2-(3-chlorophenyl)-2,2-difluoro-1-phenylethyl ((S)-1-(((S)-4-cyano-3-oxo-1-((S)-2-oxopyrrolidin-3-yl)-4-(tetrahydro-1|4-thiophen-1-ylidene) butan-2-yl)amino)-1-oxohexan-2-yl)carbamate (4). TLC system: 10% Methanol in dichloromethane Rf: 0.5 LCMS (ESI): m/z 549.3 [M+H]⁺

### (3S)-3-((2S)-2-(((2-(3-chlorophenyl)-2,2-difluoro-1-phenylethoxy) carbonyl) amino) hexanamido)-2-oxo-4-((S)-2-oxopyrrolidin-3-yl)butanoic acid: A250

To a stirred solution of 2-(3-chlorophenyl)-2,2-difluoro-1-phenylethyl ((S)-1-(((S)-4-cyano-3-oxo-1-((S)-2-oxopyrrolidin-3-yl)-4-(tetrahydro-1|4-thiophen-1-ylidene)butan-2-yl) amino)-1-oxohexan-2-yl)carbamate (4) (3.5 g, 5.079 mmol) in THE (20 mL) water (20 mL) was added Oxone (4.67 g, 15.239 mmol) at 0° C. and stirred at RT for 3 h. The progress of the reaction was monitored by TLC and LC-MS. Reaction mixture was diluted with ethyl acetate (25 mL) and washed with water (2×20 mL). Organic layer was dried over anhydrous Na₂SO₄ and concentrated to afford (3S)-3-((2S)-2-(((2-(3-chlorophenyl)-2,2-difluoro-1-phenyl ethoxy) carbonyl)amino)hexanamido)-2-oxo-4-((S)-2-oxopyrrolidin-3-yl)butanoic acid (A250). 100 mg of this material was purified by prep. HPLC to afford A250.

Prep. HPLC conditions: Column/dimensions: X BRIDGE phenyl (19*250) 5 μm Mobile Phase A: 10 mM ABC in water Mobile Phase B: 10 mM ABC Acetonitrile:MeOH (1:1) Gradient (Time/% B): 0/5, 1/5, 8/35, 13.5/35, 13.51/98, 19/98, 19.01/5, 22/5; Flow rate: 18 mL/min. Solubility: ACN. TLC system: 10% Methanol in dichloromethane Rf: 0.2 LCMS (ESI): m/z 608.3 [M+H]⁺

### Example 38: Synthesis of Compound A251

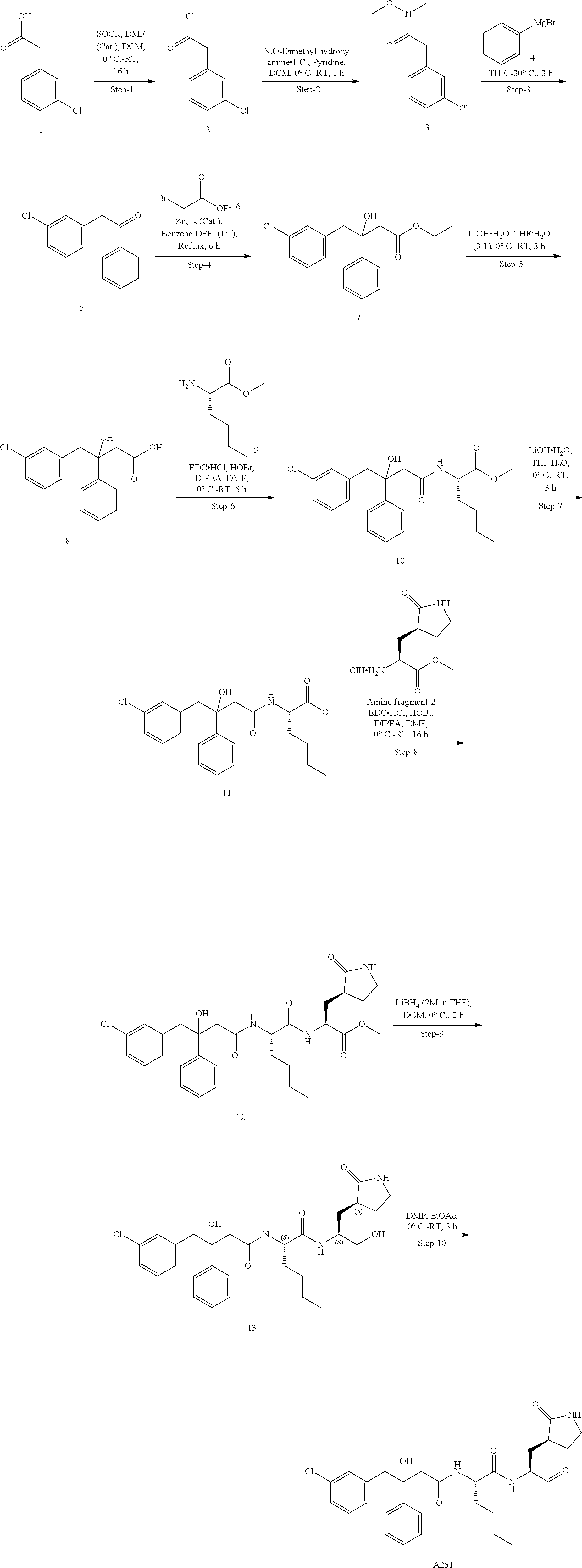

### 2-(3-chlorophenyl)acetyl chloride (2)

To a stirred solution of 2-(3-chlorophenyl)acetic acid (1) (40 g, 235.29 mmol) in DCM (400 mL) was added catalytic amount of DMF and SOCl₂ (25.6 mL, 352.94 mmol) slowly drop wise at 0° C. and stirred at RT for 16 h. Reaction progress was monitored by TLC. After completion of the reaction, reaction mixture was evaporated under reduced pressure in the presence of argon atmosphere to afford 2-(3-chlorophenyl) acetyl chloride (2) that was used directly in the next step.

### 2-(3-chlorophenyl)-N-methoxy-N-methylacetamide (3)

To a stirred solution of 2-(3-chlorophenyl)acetyl chloride (2) (44 g, 232.76 mmol) in DCM (440 mL) wad added N,O-Dimethyl hydroxyl amine·HCl (17.03 g, 279.31 mmol) at 0° C. To this added Pyridine (51 mL, 465.52 mmol) slowly drop wise at 0° C. and stirred at RT for 2 h. Reaction progress was monitored by TLC. After completion of the reaction, reaction mixture was diluted with a mixture of brine sol:DCM:DEE (2:1:1) (160 mL). Organic layer was separated, dried over anhy. Na₂SO₄ and evaporated under reduced pressure to afford 2-(3-chlorophenyl)-N-methoxy-N-methylacetamide (3). TLC system: 30% Ethyl acetate/Pet ether Rf: 0.3 2-(3-chlorophenyl)-1-phenylethan-1-one (5):

To a stirred solution of 2-(3-chlorophenyl)-N-methoxy-N-methylacetamide (3) (48 g, 224.65 mmol) in THE (480 mL) wad added PhMgBr (1M in THF, 450 mL, 449.3 mmol) slowly drop wise at −30° C. and stirred for 4 h. Reaction progress was monitored by TLC. After completion of the reaction, reaction mixture was quenched with Sat·NH₄Cl soln. and extracted with ethyl acetate (500 mL). Combined organic layer was dried over anhy Na₂SO₄ and evaporated under reduced pressure. The residue was purified by combi-flash NP, compound eluted at 1% EtoAc:hexane to afford 2-(3-chlorophenyl)-1-phenylethan-1-one (5). TLC system: 10% Ethyl acetate/Pet ether Rf: 0.3 LCMS (ESI): m/z 231.06 [M+H]⁺

### ethyl 4-(3-chlorophenyl)-3-hydroxy-3-phenylbutanoate (7)

To a stirred solution of 2-(3-chlorophenyl)-1-phenylethan-1-one (5) (10 g, 43.47 mmol) in Benzene:DEE (1:1) (100 mL) was added Zinc (freshly activated, 21.19 g, 326.08 mmol), ethyl 2-bromoacetate (19.23 mL, 173.88 mmol) followed by I₂ (Cat.) and the reaction was stirred at 70° C. for 6 h. The progress of the reaction was monitored by TLC. Reaction mass was cooled to RT and filtered on diatomaceous earth bed and washed with DEE (50 mL). Obtained filtrate was washed with water (50 mL), brine solution (50 mL), dried over anhy. sodium sulfate and concentrated under reduced pressure. The residue was purified by combi-flash NP, compound eluted at 5% EtoAc:hexane to afford ethyl 4-(3-chlorophenyl)-3-hydroxy-3-phenylbutanoate (7). TLC system: 10% EtoAc:hexane Rf: 0.2 LCMS (ESI): m/z 301.10 [M-OH]⁺

### 4-(3-chlorophenyl)-3-hydroxy-3-phenylbutanoic acid (8)

To a stirred solution of ethyl 4-(3-chlorophenyl)-3-hydroxy-3-phenylbutanoate (7) (8.0 g, 25.09 mmol) in THE (60 mL) and water (20 mL) was added LiOH·H₂O (1.58 g, 37.73 mmol) at 0° C. and stirred at room temperature for 1 h. The progress of the reaction was monitored by TLC. Excess of THE was distilled under reduced pressure, compound was acidified with aq. 1N HCl solution up to pH˜2 and extracted with ethyl acetate (2×100 mL). Combined organic layer was washed with water (100 mL), brine solution (100 mL), dried over anhy. sodium sulfate, concentrated under reduced pressure to afford 4-(3-chlorophenyl)-3-hydroxy-3-phenylbutanoic acid (8). TLC system: 100% Ethyl acetate Rf: 0.2 LCMS (ESI): m/z 288.99 [M−H]⁺

### methyl (2S)-2-(4-(3-chlorophenyl)-3-hydroxy-3-phenylbutanamido)hexanoate (10)

To a stirred solution of 4-(3-chlorophenyl)-3-hydroxy-3-phenylbutanoic acid (8)(7 g, 24.04 mmol) in DMF (70 mL) was added EDC·HCl (7 g, 36.206 mmol), HOBt (4.9 g, 36.206 mmol), DIPEA (12.6 mL, 72.41 mmol) and methyl (S)-2-aminohexanoate hydrochloride (5.25 g, 28.96 mmol) at 0° C. simultaneously and stirred at room temperature for 4 h. Reaction mixture was quenched with ice water (140 mL) and extracted with ethyl acetate (2×100 mL). Combined organic layer was washed with brine solution (2×100 mL), dried over anhy. sodium sulfate and evaporated under reduced pressure. The residue was purified by combi-flash NP, compound eluted at 100% ethyl acetate to afford methyl (2S)-2-(4-(3-chlorophenyl)-3-hydroxy-3-phenylbutanamido)hexanoate (10). TLC system: 100% Ethyl acetate Rf: 0.4 LCMS (ESI): m/z 416.15 [M−H]⁺

### (2S)-2-(4-(3-chlorophenyl)-3-hydroxy-3-phenylbutanamido)hexanoic acid (11)

To a stirred solution of methyl (2S)-2-(4-(3-chlorophenyl)-3-hydroxy-3-phenylbutanamido)hexanoate (10) (6 g, 14.36 mmol) in THE (45 mL) and water (15 mL) was added LiOH·H₂O (906.47 mg, 21.58 mmol) and stirred at room temperature for 3 h. The progress of the reaction was monitored by TLC and LCMS. Excess of THE was distilled under reduced pressure, compound acidified with aq. 1N HCl solution up to pH˜2 and extracted with ethyl acetate (2×100 mL). Combined organic layer was washed with water (100 mL), brine solution (100 mL), dried over anhy. sodium sulfate and concentrated under reduced pressure to afford (2S)-2-(4-(3-chlorophenyl)-3-hydroxy-3-phenylbutanamido)hexanoic acid (11). TLC system: 100% Ethyl acetate Rf: 0.2 LCMS (ESI): m/z 402.13 [M−H]⁺

### methyl (2S)-2-((2S)-2-(4-(3-chlorophenyl)-3-hydroxy-3-phenylbutanamido)hexanamido)-3-((S)-2-oxopyrrolidin-3-yl)propanoate (12)

To a stirred solution of (2S)-2-(4-(3-chlorophenyl)-3-hydroxy-3-phenylbutanamido)hexanoic acid (11) (5 g, 12.38 mmol) in DMF (50 mL) was added EDC·HCl (3.6 g, 18.57 mmol), HOBt (2.5 g, 18.57 mmol), DIPEA (6.5 mL, 37.14 mmol) and methyl (S)-2-amino-3-((R)-2-oxopyrrolidin-3-yl)propanoate (Amine fragment-2′) (3.3 g, 14.85 mmol) at 0° C. simultaneously and stirred at room temperature for 4 h. Reaction mixture was quenched with ice water (100 mL) and extracted with ethyl acetate (2×100 mL). Combined organic layer was washed with brine solution (2×100 mL), dried over anhy. sodium sulfate and evaporated under reduced pressure. The residue was purified by combi-flash NP, compound eluted at 100% ethyl acetate to afford methyl (2S)-2-((2S)-2-(4-(3-chlorophenyl)-3-hydroxy-3-phenylbutanamido)hexanamido)-3-((S)-2-oxopyrrolidin-3-yl)propanoate (12). TLC system: 100% Ethyl acetate Rf: 0.4 LCMS (ESI): m/z 570.56 [M−H]⁺

### (2S)-2-(4-(3-chlorophenyl)-3-hydroxy-3-phenylbutanamido)-N—((S)-1-hydroxy-3-((S)-2-oxopyrrolidin-3-yl)propan-2-yl)hexanamide (13)

To a stirred solution of methyl (2S)-2-((2S)-2-(4-(3-chlorophenyl)-3-hydroxy-3-phenylbutanamido)hexanamido)-3-((S)-2-oxopyrrolidin-3-yl)propanoate (12) (3 g, 5.25 mmol) in THE (45 mL) was added 2M LiBH₄ in THE (5.25 mL, 10.507 mmol) at 0° C. and the reaction mixture stirred for 2 h at 0° C. The progress of the reaction was monitored by TLC. Reaction mixture was quenched with sat. Ammonium chloride solution (40 mL) and extracted with DCM (2×50 mL). Combined organic layer was washed with brine solution (30 mL), dried over anhy. Na₂SO₄ and concentrated under reduced pressure. Obtained material was purified by reverse phase combiflash column chromatography by using 60% ACN:WATER as eluent to afford (2S)-2-(4-(3-chlorophenyl)-3-hydroxy-3-phenylbutanamido)-N—((S)-1-hydroxy-3-((S)-2-oxopyrrolidin-3-yl)propan-2-yl)hexanamide (13). TLC system: 100% ethyl acetate Rf: 0.3 LCMS (ESI): m/z 542.19 [M−H]⁺

### (2S)-2-(4-(3-chlorophenyl)-3-hydroxy-3-phenylbutanamido)-N—((S)-1-oxo-3-((S)-2-oxopyrrolidin-3-yl)propan-2-yl)hexanamide (A251)

To a stirred solution of (2S)-2-(4-(3-chlorophenyl)-3-hydroxy-3-phenylbutanamido)-N—((S)-1-hydroxy-3-((S)-2-oxopyrrolidin-3-yl)propan-2-yl)hexanamide (13) (1.5 g, 2.7 mmol) in Ethyl acetate (15 mL) was added Dess-Martin periodinane (1.75 g, 6.9 mmol) at 0° C. and stirred at RT for 2 h. After completion of the reaction, reaction mixture was diluted with Ethyl acetate (30 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure. The compound was purified by reverse phase column afford to (2S)-2-(4-(3-chlorophenyl)-3-hydroxy-3-phenylbutanamido)-N—((S)-1-oxo-3-((S)-2-oxopyrrolidin-3-yl)propan-2-yl)hexanamide (A251). TLC system: 5% Methanol in DCM Rf: 0.4 LCMS (ESI): m/z 542.2 [M+H]⁺

### Example 39: Synthesis of Compounds A254 and A255

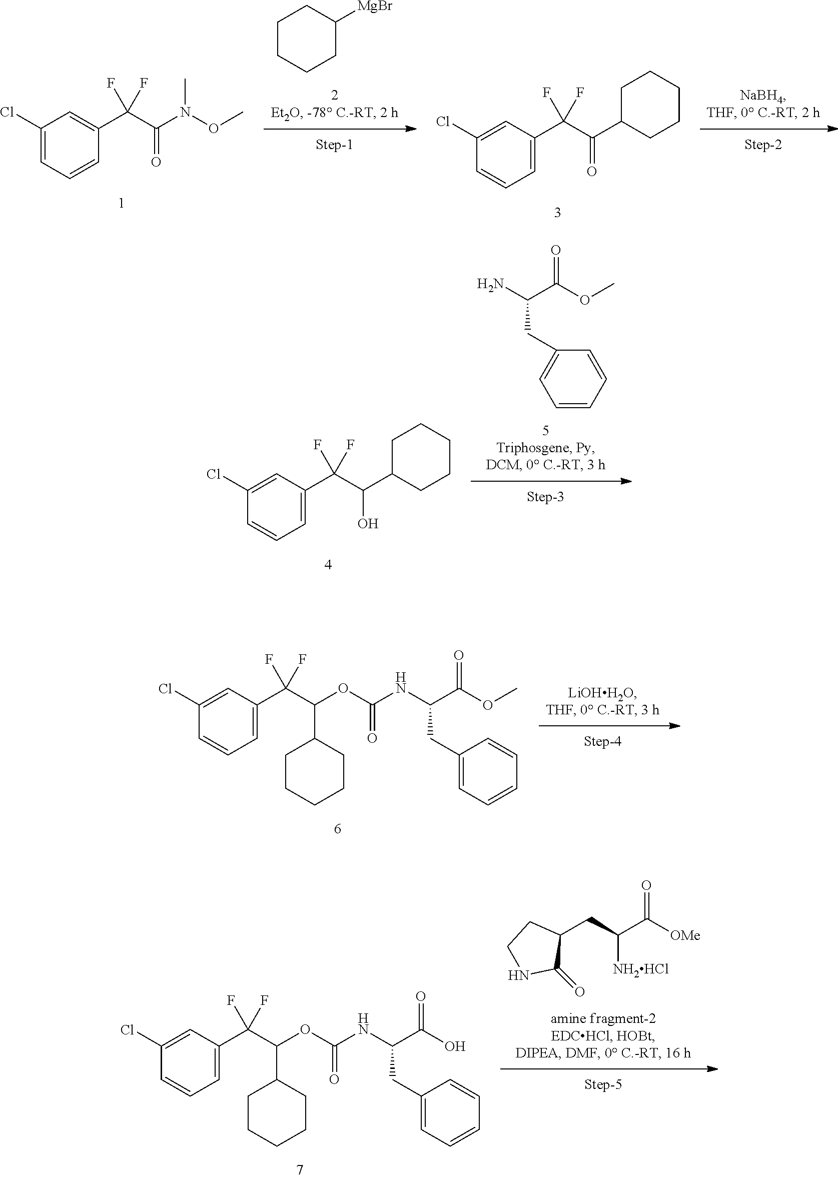

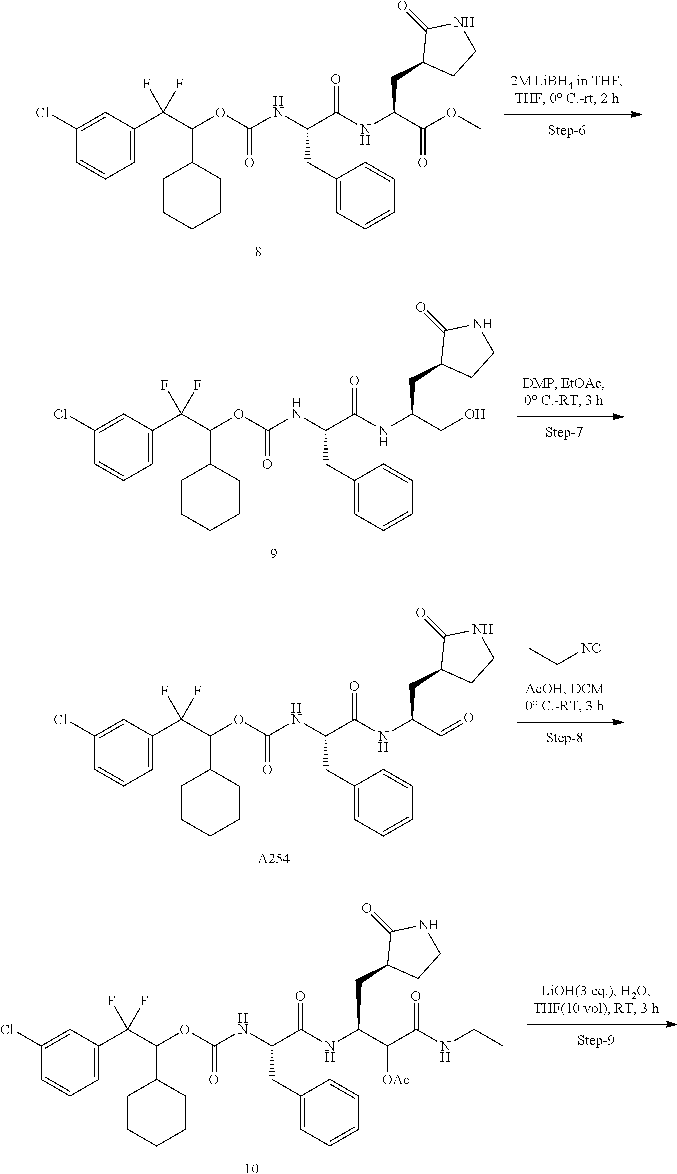

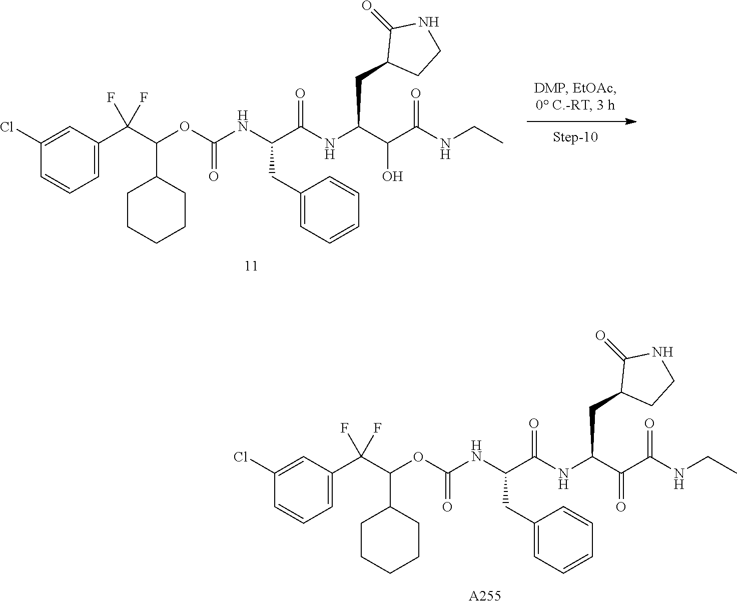 [SMILES: CCNC(=O)C(O)[C@H](C[C@@H]1CCNC1=O)NC(=O)[C@H](Cc1ccccc1)NC(=O)OC(C1CCCCC1)C(F)(F)c1cccc(Cl)c1 ; "11" ; CCNC(=O)C(=O)[C@H](C[C@@H]1CCNC1=O)NC(=O)[C@H](Cc1ccccc1)NC(=O)OC(C1CCCCC1)C(F)(F)c1cccc(Cl)c1 ; "A255"]

### 2-(3-Chlorophenyl)-1-cyclohexyl-2,2-difluoroethane-1-one

To a stirred solution of 2-(3-chlorophenyl)-2,2-difluoro-N-methoxy-N-methylacetamide (1) (6.5 g, 26.10 mmol) (1) in diethyl ether (65 mL) was added cyclohexylmagnesium bromide (52 mL, 52.20 mmol) at 0° C. The reaction mixture was allowed to stir for 2 h at RT. Reaction progress was monitored by TLC. The reaction mixture was quenched with sat. Ammonium chloride solution (100 mL) and extracted with ethyl acetate (2×100 mL). The combined organic layer was washed with water (1×50 mL), brine (1×50 mL), dried over sodium sulfate and evaporated under reduced pressure to afford 2-(3-chlorophenyl)-1-cyclohexyl-2,2-difluoroethane-1-one (3). TLC system: 20% Ethyl acetate/Pet ether Rf: 0.5 LCMS (ESI): m/z 313.14 [M+CH₃CN)⁺

### 2-(3-chlorophenyl)-1-cyclohexyl-2,2-difluoroethane-1-ol (4)

To a stirred solution of 2-(3-chlorophenyl)-1-cyclohexyl-2,2-difluoroethane-1-one (3) (5.5 g, 20.22 mmol) in THE (55 mL) wad added sodium borohydride (2.29 g, 60.66 mmol) at 0° C. and stirred at RT for 2 h. Reaction progress was monitored by TLC. Reaction mixture was quenched with H₂O (50 mL) and extracted with ethyl acetate (2×50 mL).

The combined organic layer was washed with water (40 mL), brine (40 mL), dried over sodium sulfate, evaporated under reduced pressure to afford 2-(3-chlorophenyl)-1-cyclohexyl-2,2-difluoroethane-1-ol (4). TLC system: 30% Ethyl acetate/Pet ether Rf: 0.5 LCMS (ESI): m/z 297.15 [M+Na)⁺

### Methyl ((2-(3-chlorophenyl)-1-cyclohexyl-2,2-difluoroethoxy)carbonyl)-L-phenylalaninate (6)

To a stirred solution of 2-(3-chlorophenyl)-1-cyclohexyl-2,2-difluoroethane-1-ol (4) (7.0 g, 25.54 mmol) in DCM (70 mL) was added pyridine (21 mL, 3 vol) and methyl L-phenylalaninate (5) (17.0 g, 51.09 mmol) followed by triphosgene (15.12 g, 51.09 mmol) at 0° C. portion wise and stirred at RT for 3 h. The progress of the reaction was monitored by TLC. Reaction mixture was quenched with 1N aq HCl (100 mL) and extracted with DCM (2×100 mL), dried over sodium sulfate and evaporated under reduced pressure. The material was purified by combi-flash (normal phase) by eluting with 20% ethyl acetate in hexane to afford methyl ((2-(3-chlorophenyl)-1-cyclohexyl-2,2-difluoroethoxy)carbonyl)-L-phenylalaninate (6). TLC system: 20% Ethyl acetate/Pet ether Rf: 0.5 LCMS (ESI): m/z 460.48 [M−F]⁺

### ((2-(3-Chlorophenyl)-1-cyclohexyl-2,2-difluoroethoxy)carbonyl)-L-phenylalanine (7)

To a stirred solution of methyl ((2-(3-chlorophenyl)-1-cyclohexyl-2,2-difluoroethoxy) carbonyl)-L-phenylalaninate (6) (4.0 g, 8.370 mmol) in THE (40 mL), water (20 mL) was added lithium hydroxide (1.052 g, 25.05 mmol) at 0° C. and stirred at room temperature for 3 h. The progress of the reaction was monitored by TLC and LC-MS. Excess of THE was distilled under reduced pressure, compound acidified with aq. 1N HCl solution up to pH˜2 and extracted with ethyl acetate (2×50 mL). The combined organic layer was washed with water (50 mL) brine (50 mL), dried over sodium sulfate and concentrated under reduced pressure to afford ((2-(3-chlorophenyl)-1-cyclohexyl-2,2-difluoroethoxy)carbonyl)-L-phenylalanine (7). TLC system: 5% MeOH:DCM Rf: 0.5 LCMS (ESI): m/z 446.50 [M−F]⁺

### Methyl(2S)-2-((2S)-2-(((2-(3-chlorophenyl)-1-cyclohexyl-2,2-difluoroethoxy) carbonyl)amino)-3-phenylpropanamido)-3-((S)-2-oxopyrrolidin-3-yl)propanoate (8)

To a stirred solution of ((2-(3-chlorophenyl)-1-cyclohexyl-2,2-difluoroethoxy)carbonyl)-L-phenylalanine (7) (3.8 g, 8.172 mmol) in DMF (25 mL) was added, EDC·HCl (2.341 g, 12.25 mmol), HOBt (1.65 g, 12.25 mmol), DIPEA (4.2 mL, 24.51 mmol) and methyl (S)-2-amino-3-((S)-2-oxopyrrolidin-3-yl)propanoate (Amine fragment-2) (1.82 g, 9.80 mmol) at 0° C. The reaction mixture was allowed to stir at room temperature for 16 h. Reaction mixture was quenched with ice water (150 mL) and extracted with ethyl acetate (2×100 mL). The combined organic layer was washed with brine (2×50 mL), dried over sodium sulfate and evaporated under reduced pressure. The material was purified by combi-flash (normal phase) to afford methyl (2S)-2-((2S)-2-(((2-(3-chlorophenyl)-1-cyclohexyl-2,2-difluoroethoxy)carbonyl)amino)-3-phenylpropanamido)-3-((S)-2-oxopyrrolidin-3-yl)propanoate (8). TLC system: 10% Methanol in dichloromethane Rf: 0.4 LCMS (ESI): m/z 634.67 [M+H]⁺

### 2-(3-Chlorophenyl)-1-cyclohexyl-2,2-difluoroethyl((S)-1-(((S)-1-hydroxy-3-((S)-2-oxo pyrrolidin-3-yl)propan-2-yl)amino)-1-oxo-3-phenylpropan-2-yl)carbamate (9)

To a stirred solution of methyl (2S)-2-((2S)-2-(((2-(3-chlorophenyl)-1-cyclohexyl-2,2-difluoroethoxy)carbonyl)amino)-3-phenylpropanamido)-3-((S)-2-oxopyrrolidin-3-yl)propanoate (8) (3.0 g, 4.73 mmol) in THE (10 mL) was added LiBH₄ (4.7 mL, 9.47 mmol) at 0° C. and the reaction mixture stirred for 2 h at RT. The progress of the reaction was monitored by TLC. The reaction mixture was quenched with sat. Ammonium chloride solution (50 mL) and extracted with EtOAc (2×100 mL). The combined organic layer was washed with brine (30 mL), dried over Na₂SO₄ and concentrated to afford product, which was triturated with diethyl ether (50 mL) to afford 2-(3-chlorophenyl)-1-cyclohexyl-2,2-difluoroethyl ((S)-1-(((S)-1-hydroxy-3-((S)-2-oxopyrrolidin-3-yl)propan-2-yl)amino)-1-oxo-3-phenylpropan-2-yl)carbamate (9). TLC system: 10% Methanol in DCM Rf: 0.2 LCMS (ESI): m/z 606.6 [M+H]⁺

### 2-(3-chlorophenyl)-1-cyclohexyl-2,2-difluoroethyl((S)-1-oxo-1-(((S)-1-oxo-3-((S)-2-oxopyrrolidin-3-yl)propan-2-yl)amino)-3-phenylpropan-2-yl)carbamate (A254)

To a stirred solution of methyl (2S)-2-((2S)-2-(((2-(3-chlorophenyl)-1-cyclohexyl-2,2-difluoroethoxy)carbonyl)amino)-3-phenylpropanamido)-3-((S)-2-oxopyrrolidin-3-yl)propanoate (9) (1.0 g, 1.6528 mmol) in ethyl acetate (20 mL) was added Dess-Martin periodinane (1.4 g, 3.30 mmol) at 0° C. and stirred at RT for 3 h. The reaction mixture was diluted with ethyl acetate (50 mL) and filtered through diatomaceous earth pad and washed with sat. Hypo solution (3×20 mL) followed by sat. NaHCO₃ solution (3×20 mL). Organic layer was dried over anhydrous Na₂SO₄, filtered and concentrated to get a material which was purified by combi-flash (C-18, 0.1% ammonium bicarbonate in water: acetonitrile) followed by lyophilization to afford 2-(3-chlorophenyl)-1-cyclohexyl-2,2-difluoroethyl ((S)-1-oxo-1-(((S)-1-oxo-3-((S)-2-oxopyrrolidin-3-yl)propan-2-yl)amino)-3-phenylpropan-2-yl)carbamate (A254). TLC system: 10% Methanol in DCM Rf: 0.4 LCMS (ESI): m/z 604.3 [M+H]⁺

### (6S,9S)-6-benzyl-1-(3-chlorophenyl)-2-cyclohexyl-1,1-difluoro-4,7,11-trioxo-9-(((S)-2-oxopyrrolidin-3-yl)methyl)-3-oxa-5,8,12-triazatetradecan-10-yl acetate (10)

To a stirred solution of 2-(3-chlorophenyl)-1-cyclohexyl-2,2-difluoroethyl ((S)-1-oxo-1-(((S)-1-oxo-3-((S)-2-oxopyrrolidin-3-yl) propan-2-yl)amino)-3-phenylpropan-2-yl)carbamate (A254) (250 mg, 0.414 mmol) in DCM (5 mL) was added isocyanoethane (1 mL, 4 vol) at 0° C. followed by Acetic acid (0.05 mL, 0.83 mmol) at 0° C. and stirred at RT for 3 h. The progress of the reaction was monitored by TLC and LC-MS. Reaction mixture was diluted with dichloromethane (20 mL) and washed with sat. NaHCO₃ solution (3×10 mL) followed by brine (1×10 mL). The organic layer was dried over anhydrous Na₂SO₄ and evaporated under reduced pressure to afford (6S,9S)-6-benzyl-1-(3-chlorophenyl)-2-cyclohexyl-1,1-difluoro-4,7,11-trioxo-9-(((S)-2-oxopyrrolidin-3-yl)methyl)-3-oxa-5,8,12-triazatetradecan-10-yl acetate (10). TLC system: 10% Methanol in dichloromethane Rf: 0.4 LCMS (ESI): m/z 719.40 [M+H]⁺

### 2-(3-chlorophenyl)-1-cyclohexyl-2,2-difluoroethyl ((2S)-1-(((2S)-4-(ethylamino)-3-hydroxy-4-oxo-1-((S)-2-oxopyrrolidin-3-yl)butan-2-yl)amino)-1-oxo-3-phenylpropan-2-yl)carbamate (11)

To a stirred solution of (6S,9S)-6-benzyl-1-(3-chlorophenyl)-2-cyclohexyl-1,1-difluoro-4,7,11-trioxo-9-(((S)-2-oxopyrrolidin-3-yl)methyl)-3-oxa-5,8,12-triazatetradecan-10-yl acetate (10) (160 mg, 0.2228 mmol) in THE (1.6 mL), water (0.8 mL) was added lithium hydroxide (28 mg, 0.6685 mmol) at 0° C. and stirred at room temperature for 3 h. The progress of the reaction was monitored by TLC. Solvent was removed under reduced pressure, compound acidified with aq. 1N HCl solution up to pH˜2 and extracted with ethyl acetate (2×10 mL), dried over sodium sulfate, concentrated under reduced pressure to afford 2-(3-chlorophenyl)-1-cyclohexyl-2,2-difluoroethyl((2S)-1-(((2S)-4-(ethylamino)-3-hydroxy-4-oxo-1-((S)-2-oxopyrrolidin-3-yl)butan-2-yl) amino)-1-oxo-3-phenylpropan-2-yl)carbamate (11). TLC system: 10% Methanol in dichloromethane Rf: 0.4 LCMS (ESI): m/z 677.3 (M+H)⁺

### 2-(3-Chlorophenyl)-1-cyclohexyl-2,2-difluoroethyl ((S)-1-(((S)-4-(ethylamino)-3,4-dioxo-1-((S)-2-oxopyrrolidin-3-yl)butan-2-yl)amino)-1-oxo-3-phenylpropan-2-yl)carbamate (A255)

To a stirred solution of 2-(3-chlorophenyl)-1-cyclohexyl-2,2-difluoroethyl ((2S)-1-(((2S)-4-(ethylamino)-3-hydroxy-4-oxo-1-((S)-2-oxopyrrolidin-3-yl)butan-2-yl)amino)-1-oxo-3-phenylpropan-2-yl)carbamate (11) (100 mg, 0.147 mmol) in ethyl acetate (5 mL) was added Dess-Martin periodinane (156 mg, 0.37 mmol) at 0° C. and stirred at RT for 3 h. progress of the reaction monitored by TLC. The suspension was filtered through diatomaceous earth bed and washed with ethyl acetate (20 mL), the filtrate was washed with sat. Hypo solution (3×25 mL) followed by sat. NaHCO₃ solution (3×25 mL) and brine (25 mL). Organic layer was dried over anhydrous Na₂SO₄ and concentrated to afford material, which was triturated with Et2O/pentane (10 mL) to afford 2-(3-chlorophenyl)-1-cyclohexyl-2,2-difluoroethyl ((S)-1-(((S)-4-(ethylamino)-3,4-dioxo-1-((S)-2-oxopyrrolidin-3-yl)butan-2-yl)amino)-1-oxo-3-phenylpropan-2-yl)carbamate (A255). TLC system: 10% Methanol in dichloromethane Rf: 0.5 LCMS (ESI): m/z 675.3 (M+H)⁺

### Example 40: Synthesis of Compounds A256 and A257

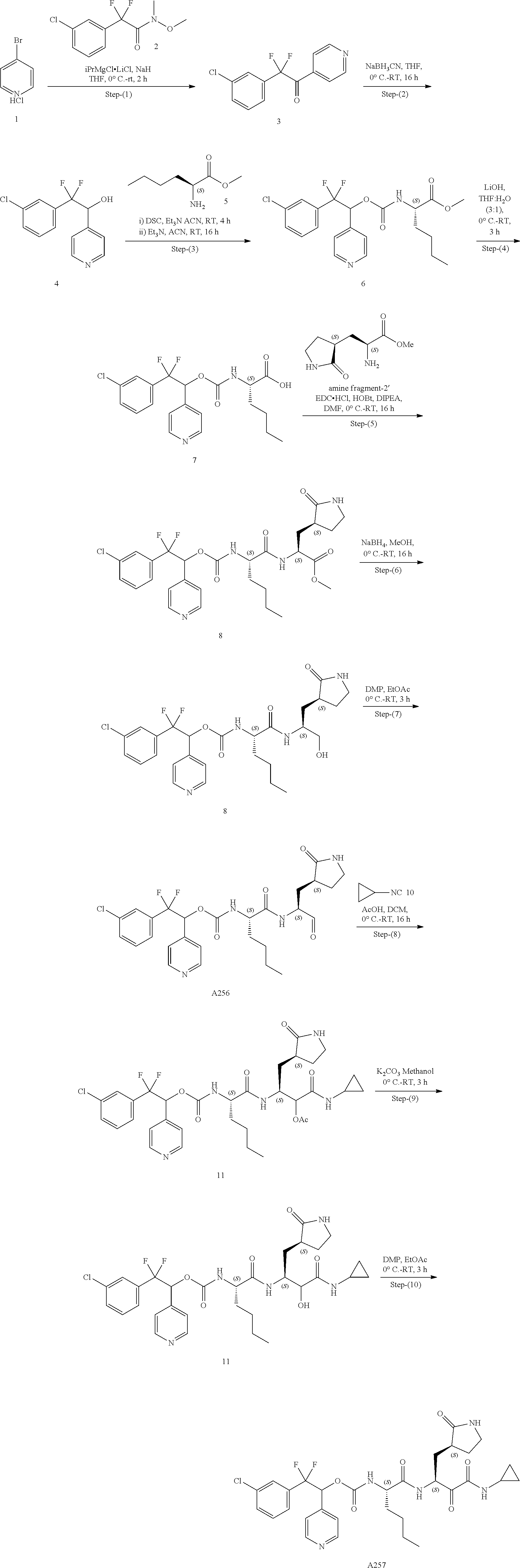

### 2-(3-chlorophenyl)-2,2-difluoro-1-(pyridin-4-yl)ethan-1-one (3)

To a stirred solution of 4-bromopyridine hydrochloride (1) (5.0 g, 25.773 mmol) in THE was added iPrMgCl·LiCl (40 mL, 51.546 mmol) at 0° C. The reaction mixture was allowed to stir at rt for 2 h. After 2 h, then was added 2-(3-chlorophenyl)-2,2-difluoro-N-methoxy-N-methylacetamide (2) (6.41 g, 25.773 mmol) at 0° C. and stirred at RT for 2 h. Reaction progress was monitored by TLC. The reaction mixture was quenched with sat. Ammonium chloride solution (100 mL) and extracted with ethyl acetate (2×50 mL). The combined the organic layer was washed with water (1×50 mL), brine (1×50 mL), dried over sodium sulfate and evaporated under reduced pressure to afford 2-(3-chlorophenyl)-2,2-difluoro-1-(pyridin-4-yl)ethan-1-one (3). TLC system: 40% Ethyl acetate/Pet ether Rf: 0.3 LCMS (ESI): m/z 286.02 [M+H₂O]⁺

### 2-(3-Chlorophenyl)-2,2-difluoro-1-(pyridin-4-yl)ethan-1-ol (4)

To a stirred solution of 2-(3-chlorophenyl)-2,2-difluoro-1-(pyridin-4-yl)ethan-1-one (3) (1.5 g, 5.617 mmol) in methanol (15 mL) wad added NaBH₃CN (1.41 g, 22.471 mmol) at 0° C. and stirred at RT for 16 h. Reaction progress was monitored by TLC. The reaction mixture was quenched with water (20 mL) and extracted with ethyl acetate (2×50 mL). The combined organic layer was washed with water (1×40 mL), brine (1×40 mL) and dried over sodium sulfate, evaporated under reduced pressure to afford 2-(3-chlorophenyl)-2,2-difluoro-1-(pyridin-4-yl)ethan-1-ol (4). TLC system: 30% Ethyl acetate/Pet ether Rf: 0.3 LCMS (ESI): m/z 270.1 [M+H]

### Methyl (2S)-2-(((2-(3-chlorophenyl)-2,2-difluoro-1-(pyridin-4-yl)ethoxy)carbonyl)amino)hexanoate (6)

To a stirred solution of 2-(3-chlorophenyl)-2,2-difluoro-1-(pyridin-4-yl)ethan-1-ol (4) (1 g, 3.703 mmol) in ACN (10 mL) was added N,N′disuccinamidyl carbonate (2.4 g, 9.259 mmol), followed by Et₃N (1.56 mL, 11.109 mmol) at 0° C. and stirred at room temperature for 4 h. The progress of the reaction was monitored by TLC. The reaction mass was used directly in the subsequent reaction.

In another RB flask, methyl (S)-2-aminohexanoate hydrochloride (5) (1.34 g, 7.407 mmol) was taken in ACN (10 mL), and treated with Et₃N (1.56 mL, 11.109 mmol). The reaction mixture was stirred for 10 min, then the above prepared reaction mass was added drop-wise and the reaction mixture stirred at room temperature for 16 h. Reaction mixture was quenched with ice water (50 mL) and extracted with ethyl acetate (2×50 mL). The combined organic layer was washed with brine (50 mL), dried over sodium sulfate and evaporated under reduced pressure to get the material, which was purified by combi-flash (normal phase) using ethyl acetate and pet ether as a mobile phases to afford methyl (2S)-2-(((2-(3-chlorophenyl)-2,2-difluoro-1-(pyridin-4-yl)ethoxy)carbonyl)amino)hexanoate (6). TLC system: 50% Ethyl acetate/Pet ether Rf: 0.3 LCMS (ESI): m/z 441.19 [M+H]⁺

### (2S)-2-(((2-(3-Chlorophenyl)-2,2-difluoro-1-(pyridin-4-yl)ethoxy)carbonyl)amino)hexanoic acid (7)

To a stirred solution of methyl (2S)-2-(((2-(3-chlorophenyl)-2,2-difluoro-1-(pyridin-4-yl)ethoxy) carbonyl)amino)hexanoate (6) (1.3 g, 2.947 mmol) in THE (13 mL), water (5 mL) was added lithium hydroxide (142 mg, 5.895 mmol) at RT and stirred at room temperature for 3 h. The progress of the reaction was monitored by TLC. Excess of THE was distilled under reduced pressure, compound acidified with aq. 1N HCl solution up to pH˜2 and extracted with ethyl acetate (2×50 mL). The combined organic layer was washed with water (25 mL) brine (25 mL), dried over sodium sulfate, concentrated under reduced pressure to afford (2S)-2-(((2-(3-chlorophenyl)-2,2-difluoro-1-(pyridin-4-yl)ethoxy)carbonyl)amino)hexanoic acid (7). TLC system: 70% Ethyl acetate/Pet ether Rf: 0.2 LCMS (ESI): m/z 427.13 [M+H]⁺

### Methyl (2S)-2-((2S)-2-(((2-(3-chlorophenyl)-2,2-difluoro-1-(pyridin-4-yl)ethoxy)carbonyl)amino)hexanamido)-3-((S)-2-oxopyrrolidin-3-yl)propanoate (8)

To a stirred solution of (2S)-2-(((2-(3-chlorophenyl)-2,2-difluoro-1-(pyridin-4-yl)ethoxy) carbonyl)amino)hexanoic acid (7) (1.2 g, 2.810 mmol) in DMF (12 mL) was added EDC·HCl (805 mg, 4.215 mmol), HOBt (570 mg, 4.215 mmol), DIPEA (1.5 mL, 8.430 mmol) and methyl (S)-2-amino-3-((S)-2-oxopyrrolidin-3-yl)propanoate (Amine fragment-2) (750 mg, 3.372 mmol) at 0° C. and stirred at room temperature for 16 h. Reaction mixture was quenched with ice water (30 mL) and extracted with ethyl acetate (2×30 mL). The combined organic layer was washed with brine (2×50 mL), dried over sodium sulfate and evaporated under reduced pressure. The material was purified by combi-flash (normal phase), compound eluted at 80% ethyl acetate to afford ethyl (2S)-2-((2S)-2-(((2-(3-chlorophenyl)-2,2-difluoro-1-(pyridin-4-yl)ethoxy) carbonyl)amino)hexanamido)-3-((S)-2-oxopyrrolidin-3-yl)propanoate (8). TLC system: 10% Methanol in DCM Rf: 0.4 LCMS (ESI): m/z 595.3 [M+H]⁺

### 2-(3-Chlorophenyl)-2,2-difluoro-1-(pyridin-4-yl)ethyl ((S)-1-(((S)-1-hydroxy-3-((S)-2-oxopyrrolidin-3-yl)propan-2-yl)amino)-1-oxohexan-2-yl)carbamate (9)

To a stirred solution of methyl (2S)-2-((2S)-2-(((2-(3-chlorophenyl)-2,2-difluoro-1-(pyridin-4-yl)ethoxy)carbonyl)amino)hexanamido)-3-((S)-2-oxopyrrolidin-3-yl)propanoate (8) (900 mg, 1.512 mmol) in MeOH (10 mL) was added NaBH₄ (862 mg, 22.689 mmol) at 0° C. and the reaction mixture stirred for 16 h at RT. The progress of the reaction was monitored by TLC and LC-MS. Reaction mixture was quenched with sat. Ammonium chloride solution (20 mL) and extracted with DCM (2×20 mL). Organic layer was washed with brine (30 mL), dried over Na₂SO₄ and concentrated to afford product, this was triturated with diethyl ether (10 mL) to afford 2-(3-chlorophenyl)-2,2-difluoro-1-(pyridin-4-yl)ethyl ((S)-1-(((S)-1-hydroxy-3-((S)-2-oxopyrrolidin-3-yl)propan-2-yl)amino)-1-oxohexan-2-yl)carbamate (9). TLC system: 10% Methanol in DCM Rf: 0.2 LCMS (ESI): m/z 567.23 [M+H]⁺

### 2-(3-Chlorophenyl)-2,2-difluoro-1-(pyridin-4-yl)ethyl ((S)-1-oxo-1-(((S)-1-oxo-3-((S)-2-oxopyrrolidin-3-yl)propan-2-yl)amino)hexan-2-yl)carbamate (A256)

To a stirred solution of 2-(3-chlorophenyl)-2,2-difluoro-1-(pyridin-4-yl)ethyl ((S)-1-(((S)-1-hydroxy-3-((S)-2-oxopyrrolidin-3-yl)propan-2-yl)amino)-1-oxohexan-2-yl)carbamate (9) (350 mg, 0.617 mmol) in ethyl acetate (10.5 mL) was added Dess-Martin periodinane (523 mg, 1.234 mmol) at 0° C. and stirred at RT for 3 h. Reaction mixture was diluted with ethyl acetate (20 mL) and filtered through calcite pad and washed with sat. Hypo solution (3×20 mL) followed by sat. NaHCO₃ solution (3×20 mL). Organic layer was dried over anhydrous Na₂SO₄, filtered and concentrated to get material which was purified by combi-flash (C-18, 0.1% ABC in water/acetonitrile) to obtain 2-(3-cyclopropylphenyl)-2,2-difluoro-1-phenylethyl ((S)-4-methyl-1-oxo-1-(((S)-1-oxo-3-((S)-2-oxo pyrrolidin-3-yl) propan-2-yl)amino)pentan-2-yl)carbamate (A256). TLC system: 10% Methanol in DCM Rf: 0.4 LCMS (ESI): m/z 565.2 [M+H]⁺

### (3S)-3-((2S)-2-(((2-(3-Chlorophenyl)-2,2-difluoro-1-(pyridin-4-yl)ethoxy)carbonyl)amino)hexanamido)-1-(cyclopropylamino)-1-oxo-4-((S)-2-oxopyrrolidin-3-yl)butan-2-yl acetate (11)

To a stirred solution of 2-(3-chlorophenyl)-2,2-difluoro-1-(pyridin-4-yl)ethyl ((S)-1-oxo-1-(((S)-1-oxo-3-((S)-2-oxopyrrolidin-3-yl)propan-2-yl)amino)hexan-2-yl)carbamate (A256) (150 mg, 0.265 mmol) in DCM (3 mL) was added isocyanocyclopropane (10) (36 mg, 0.530 mmol) followed by acetic acid (0.2 mL, 0.530 mmol) at 0° C. and stirred at RT for 16 h. The progress of the reaction was monitored by TLC and LC-MS. The reaction mixture was diluted with dichloromethane (10 mL) and washed with 1N HCl (2×10 mL) followed by brine (1×10 mL). Organic layer was dried over anhydrous Na₂SO₄ and evaporated under reduced pressure to afford (3S)-3-((2S)-2-(((2-(3-chlorophenyl)-2,2-difluoro-1-(pyridin-4-yl)ethoxy)carbonyl)amino)hexanamido)-1-(cyclopropylamino)-1-oxo-4-((S)-2-oxopyrrolidin-3-yl)butan-2-yl acetate (11). TLC system: 10% Methanol in dichloromethane Rf: 0.5 LCMS (ESI): m/z 692.33 [M+H]⁺

### 2-(3-Chlorophenyl)-2,2-difluoro-1-(pyridin-4-yl)ethyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (12)

To a stirred solution of (3S)-3-((2S)-2-(((2-(3-chlorophenyl)-2,2-difluoro-1-(pyridin-4-yl)ethoxy)carbonyl)amino)hexanamido)-1-(cyclopropylamino)-1-oxo-4-((S)-2-oxopyrrolidin-3-yl)butan-2-yl acetate (11) (150 mg, 0.216 mmol) in MeOH (4.5 mL) was added K₂CO₃ (60 mg, 0.433 mmol) at RT and stirred at room temperature for 3 h. The progress of the reaction was monitored by TLC and LC-MS. The reaction mixture was completely distilled under reduced pressure and diluted with water and extracted with ethyl acetate (2×25 mL), dried over sodium sulfate, concentrated under reduced pressure to afford 2-(3-chlorophenyl)-2,2-difluoro-1-(pyridin-4-yl)ethyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (12). TLC system: 10% Methanol in dichloromethane Rf: 0.5 LCMS (ESI): m/z 650.7 [M+H]

### 2-(3-Chlorophenyl)-2,2-difluoro-1-(pyridin-4-yl)ethyl ((S)-1-(((S)-4-(cyclopropylamino)-3,4-dioxo-1-((S)-2-oxopyrrolidin-3-yl)butan-2-yl)amino)-1-oxohexan-2-yl)carbamate (A257)

To a stirred solution of 2-(3-chlorophenyl)-2,2-difluoro-1-(pyridin-4-yl)ethyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (12) (100 mg, 0.153 mmol) in ethyl acetate (3 mL) was added Dess-Martin periodinane (130 mg, 0.307 mmol) at 0° C. and stirred at RT for 3 h. The progress of the reaction was monitored by TLC and LC-MS. Reaction mixture was filter through diatomaceous earth pad and washed with ethyl acetate (20 mL) and filtrate was washed with hypo solution (3×15 mL) followed by saturated NaHCO₃ solution (3×15 mL). Organic layer was dried over anhydrous Na₂SO₄, filtered and concentrated to get material, which was purified by combi-flash (C-18, 0.1% ABC in water, acetonitrile) to afford 2-(3-chlorophenyl)-2,2-difluoro-1-(pyridin-4-yl)ethyl((S)-1-(((S)-4-(cyclopropylamino)-3,4-dioxo-1-((S)-2-oxopyrrolidin-3-yl)butan-2-yl)amino)-1-oxohexan-2-yl)carbamate (A257). TLC system: 10% Methanol in dichloromethane Rf: 0.4 LCMS (ESI): m/z 648.3 [M+H]⁺

### Example 41: Synthesis of Compound A258

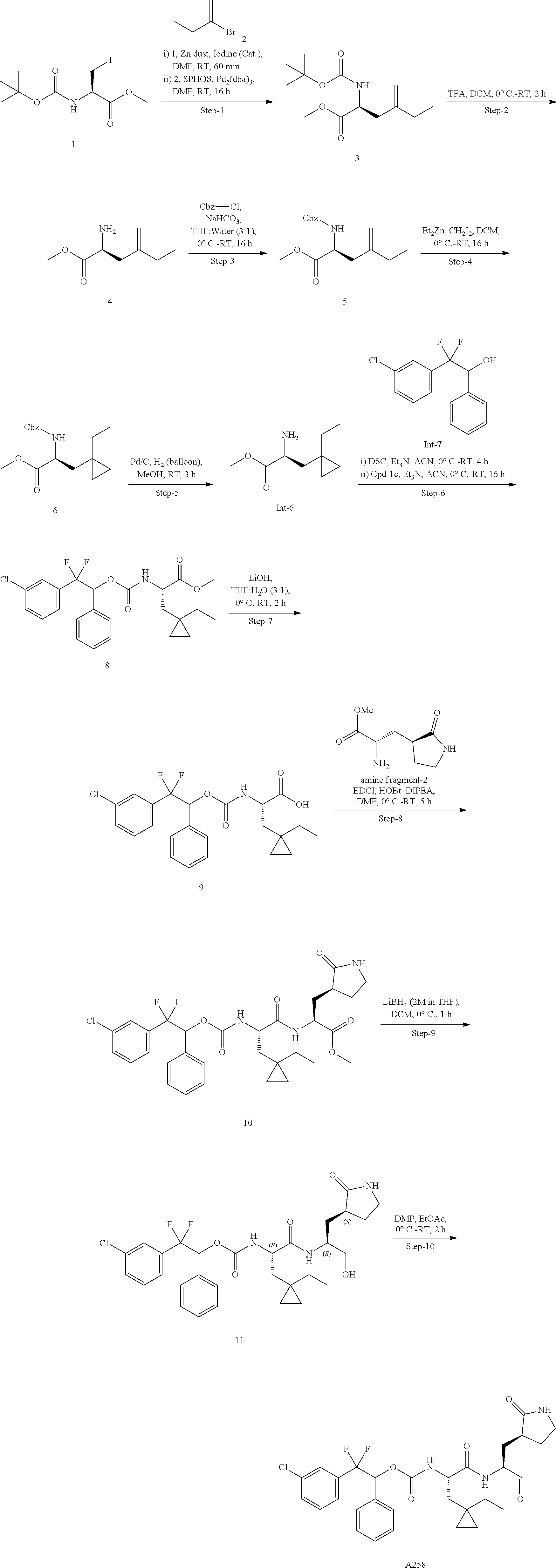

### methyl (S)-2-((tert-butoxycarbonyl)amino)-4-methylenehexanoate (3)

To a suspension of activated zinc (7.6 g, 91.1 mmol) in DMF (50 ml) was added catalytic amount of Iodine at RT and stirred for 10 min and then added methyl (R)-2-((tert-butoxy carbonyl) amino)-3-iodopropanoate (1) (10 g, 30.3 mmol) portion wise at same temperature followed by addition of catalytic amount of iodine and stirred for 60 min at same temperature. Reaction mass was degassed with argon balloon for 15 min. and added 2-bromobut-1-ene (2) (4.8 g, 33.3 mmol), Pd₂(dppf)Cl₂ (440 mg, 0.60 mmol) and SPHOS (240 mg, 0.60 mmol) at RT and heated to 50° C. for 16 h. The progress of the reaction was monitored by TLC and LCMS. After completion of starting material, reaction mixture was filtered through diatomaceous earth bed and bed was washed with ethyl acetate. Obtained filtrate was diluted with water (300 mL) and extracted with ethyl acetate (2×200 mL). Combined organic layer was washed with brine solution (250 mL), dried over anhydrous sodium sulfate and evaporated under reduced pressure. The residue was purified by column chromatography using silica gel (100-200 mesh) and 10% EtOAc:Pet.Ether as eluent to afford methyl (S)-2-((tert-butoxycarbonyl)amino)-4-methylenehexanoate (3). TLC system: 10% Ethyl acetate in hexane Rf: 0.6 methyl (S)-2-amino-4-methylenehexanoate (4):

To a stirred solution of methyl (S)-2-((tert-butoxycarbonyl)amino)-4-methylenehexanoate (3) (15.0 g, 58.3 mmol) in DCM (70 mL) was added TFA (15 mL) at 0° C. and stirred at RT for 2 h. The progress of the reaction was monitored by TLC. After completion of starting material, reaction mixture was concentrated under reduced pressure to afford methyl (S)-2-amino-4-methylenehexanoate (4) as TFA salt. TLC system: 20% Ethyl acetate in hexane Rf: 0.1

### methyl(S)-2-(((benzyloxy)carbonyl)amino)-4-methylenehexanoate (5)

To a stirred solution of methyl (S)-2-amino-4-methylenehexanoate (4) (10.0 g, 63.6 mmol) in THE (60 mL) and water (30 ml) was added NaHCO₃ (21.37 g, 254.4 mmol) at RT and Cbz-Cl (11.8 g 69.96 mmol) at 0° C. and stirred at RT for 16 h. The progress of the reaction was monitored by TLC and LCMS. After completion of starting material, reaction mixture was diluted with ice cold water (100 mL) and extracted with EtOAc (2×500 mL) and combined organic layer was washed with brine solution (300 mL), dried over anhy·Na₂SO₄ and evaporated under reduced pressure. Obtained material was purified by normal phase chromatography using silica gel (100-200 mesh) and 15% EtOAc/Pet.ether as eluent to afford methyl (S)-2-(((benzyloxy) carbonyl)amino)-4-methylenehexanoate (5). TLC system: 10% Ethyl acetate in hexane Rf: 0.5

### methyl (S)-2-(((benzyloxy)carbonyl)amino)-3-(1-ethylcyclopropyl)propanoate (6)

To a stirred solution of methyl (S)-2-(((benzyloxy)carbonyl)amino)-4-methylenehexanoate (5) (11 g, 37.5 mmol) in DCM (110 mL) was added Et₂Zn (1M in THF, 150 mL, 150 mmol) slowly drop wise at 0° C. and stirred for 10 min. After that added CH₂I₂ (24.8 g, 93.75 mmol) slowly drop wise at 0° C. and stirred at RT for 16 h. After completion of the reaction (monitored by TLC), the reaction mixture was quenched with 1N HCl solution (30 mL) at 0° C. and was extracted with ethyl acetate (2×100 mL). Combined organic layer and washed with water (100 mL), dried over anhy.

Na₂SO₄ and concentrated under reduced pressure. Obtained residue was purified by column chromatography using silica gel (100-200 mesh) and 20% ethyl acetate in Pet.ether as eluent to afford methyl (S)-2-(((benzyloxy)carbonyl)amino)-3-(1-ethylcyclopropyl)propanoate (6). TLC system: 10% Ethyl acetate Rf: 0.3 LCMS (ESI): m/z 306.4 [M+H]⁺

### methyl (S)-2-amino-3-(1-ethylcyclopropyl)propanoate (Int-6)

To a degassed solution of methyl (S)-2-(((benzyloxy)carbonyl)amino)-3-(1-ethylcyclopropyl) propanoate (6) (2.9 g, 9.5 mmol) in MeOH (29 mL) was added Pd/C (580 mg, 20% w/w) at RT and stirred for 3 h under H₂ balloon pressure. Reaction progress was monitored by TLC. After completion of starting material, reaction mass was filtered through diatomaceous earth pad and bed washed with MeOH (50 mL). Obtained filtrate was concentrated under reduced pressure to afford methyl (S)-2-amino-3-(1-ethylcyclopropyl)propanoate (Int-6). TLC system: 20% Ethyl acetate in hexane Rf: 0.1

### methyl (2S)-2-(((2-(3-chlorophenyl)-2,2-difluoro-1-phenylethoxy)carbonyl)amino)-3-(1-ethylcyclopropyl)propanoate (8)

To a stirred solution of 2-(3-chlorophenyl)-2,2-difluoro-1-phenylethan-1-ol (Int-7) (2.0 g, 7.4 mmol) in ACN (20 mL) was added N, N′-disuccinamidyl carbonate (4.76 g, 18.6 mmol) followed by triethylamine (4.9 mL, 37.3 mmol) at 0° C. and stirred the reaction mixture at room temperature for 4 h. The progress of the reaction was monitored by TLC. The reaction mass was used directly in the subsequent reaction.

In another RB flask, methyl (S)-2-amino-3-(1-ethylcyclopropyl)propanoate (Int-6) (1.53 g, 8.9 mmol) in ACN (20 mL) was treated with triethylamine (4.9 ml, 37.3 mmol). The resulting reaction mixture was stirred for 5 min, then added above prepared reaction mass drop-wise at 0° C. and the reaction mixture was stirred at room temperature for 16 h. Reaction mixture was quenched with ice water (100 mL) and extracted with ethyl acetate (2×100 mL). Combined organic layer was washed with water (100 mL), dried over anhy. Na₂SO₄ and concentrated under reduced pressure. Obtained residue was purified by column chromatography using silica gel (100-200 mesh) and 20% ethyl acetate/Pet.ether as eluent to afford methyl (2S)-2-(((2-(3-chlorophenyl)-2,2-difluoro-1-phenylethoxy)carbonyl)amino)-3-(1-ethylcyclopropyl)propanoate (8). TLC system: 10% Ethyl acetate in hexane Rf: 0.55 LCMS (ESI): m/z 466.17 [M+H]⁺

### (2S)-2-(((2-(3-chlorophenyl)-2,2-difluoro-1-phenylethoxy)carbonyl)amino)-3-(1-ethylcyclopropyl)propanoic acid (9)

To a stirred solution of methyl (2S)-2-(((2-(3-chlorophenyl)-2,2-difluoro-1-phenyl ethoxy)carbonyl)amino)-3-(1-ethylcyclopropyl)propanoate (8) (1.8 g, 3.86 mmol) in THE (12 mL) and water (6 mL) was added LiOH·H₂O (487 mg, 11.6 mmol) at 0° C. and stirred at room temperature for 2 h. The progress of the reaction was monitored by TLC. THE was distilled under reduced pressure, compound acidified with aq. 1N HCl solution up to pH˜2 and extracted with DCM (2×100 mL). Combined organic layer was washed with water (100 mL), brine solution (150 mL), dried over any. sodium sulfate and concentrated under reduced pressure to afford (2S)-2-(((2-(3-chlorophenyl)-2,2-difluoro-1-phenylethoxy)carbonyl)amino)-3-(1-ethylcyclopropyl) propanoic acid (9). TLC system: 10% Methanol in dichloromethane Rf: 0.1 LCMS (ESI): m/z 452.2 [M+H]⁺

### methyl (2S)-2-((2S)-2-(((2-(3-chlorophenyl)-2,2-difluoro-1-phenylethoxy)carbonyl)amino)-3-(1-ethylcyclopropyl)propanamido)-3-((S)-2-oxopyrrolidin-3-yl)propanoate (10)

To a stirred solution of (2S)-2-(((2-(3-chlorophenyl)-2,2-difluoro-1-phenylethoxy) carbonyl)amino)-3-(1-ethylcyclopropyl)propanoic acid (9) (1.4 g, 3.1 mmol) in DMF (14 mL) was added EDC·HCl (0.89 g, 4.6 mmol), HOBt (0.71 g, 4.6 mmol), DIPEA (2.7 mL, 15.5 mmol) and methyl (S)-2-amino-3-((R)-2-oxopyrrolidin-3-yl)propanoate (Amine fragment-2) (0.89 g, 4.0 mmol) simultaneously at 0° C. and stirred at room temperature for 5 h. After completion of the reaction (monitored by TLC), the reaction mixture was quenched with ice water (30 mL) and extracted with ethyl acetate (2×80 mL). Combined the organic layer and washed with brine solution (2×50 mL), dried over sodium sulfate and evaporated under reduced pressure. The residue was purified by combi-flash column and compound eluted at 40% ethyl acetate in Pet.ether to afford methyl (2S)-2-((2S)-2-(((2-(3-chlorophenyl)-2,2-difluoro-1-phenylethoxy)carbonyl)amino)-3-(1-ethylcyclopropyl) propan amido)-3-((S)-2-oxopyrrolidin-3-yl)propanoate (9). TLC system: 100% Ethyl acetate Rf: 0.4 LCMS (ESI): m/z 620.32 [M+H]⁺

### 2-(3-chlorophenyl)-2,2-difluoro-1-phenylethyl ((S)-3-(1-ethylcyclopropyl)-1-(((S)-1-hydroxy-3-((S)-2-oxopyrrolidin-3-yl)propan-2-yl)amino)-1-oxopropan-2-yl)carbamate (11)

To a stirred solution of methyl (2S)-2-((2S)-2-(((2-(3-chlorophenyl)-2,2-difluoro-1-phenylethoxy) carbonyl)amino)-3-(1-ethylcyclopropyl)propanamido)-3-((S)-2-oxopyrrolidin-3-yl)propanoate (10) (260 mg, 0.41 mmol) in DCM (2.6 mL) was added LiBH₄ (2M in THF) (0.4 mL, 0.83 mmol) slowly drop wise at 0° C. and stirred for 1 h at 0° C. The progress of the reaction was monitored by TLC. After completion of starting material, reaction mixture was quenched with saturated NH₄Cl solution and extracted with dichloromethane (2×100 mL). Combined organic layer was washed with water, dried over anhydrous sodium sulfate and concentrated under reduced pressure and purified by reverse phase column (C18) eluting with 50% ACN in 0.1% FA in water to afford 2-(3-chlorophenyl)-2,2-difluoro-1-phenylethyl ((S)-3-(1-ethylcyclopropyl)-1-(((S)-1-hydroxy-3-((S)-2-oxopyrrolidin-3-yl)propan-2-yl)amino)-1-oxopropan-2-yl)carbamate (10). TLC system: 5% Methanol in dichloromethane Rf: 0.35 LCMS (ESI): m/z 592.8 [M+H]⁺

### 2-(3-chlorophenyl)-2,2-difluoro-1-phenylethyl ((S)-3-(1-ethylcyclopropyl)-1-oxo-1-(((S)-1-oxo-3-((S)-2-oxopyrrolidin-3-yl)propan-2-yl)amino)propan-2-yl)carbamate (A258)

To a stirred solution of 2-(3-chlorophenyl)-2,2-difluoro-1-phenylethyl ((S)-3-(1-ethylcyclopropyl)-1-(((S)-1-hydroxy-3-((S)-2-oxopyrrolidin-3-yl)propan-2-yl)amino)-1-oxopropan-2-yl)carbamate (10) (130 mg, 0.21 mmol) in ethyl acetate (1.3 mL) was added Dess-Martin period inane (186 mg, 0.43 mmol) slowly portion wise at 0° C. and stirred at RT for 2 h. The progress of the reaction was monitored by TLC and LCMS. Reaction mixture was diluted with Ethyl acetate (50 mL) and filtered through diatomaceous earth pad and filtrate was washed with sat. sodium thiosulfate solution (3×20 mL) followed by sat. NaHCO₃ solution (3×20 mL). Organic layer was dried over anhydrous Na₂SO₄, filtered and concentrated under reduced pressure. Obtained material was purified by reverse phase combiflash column (C18) using 50% ACN in 0.1% aq. NH₄CO₃ solution as eluent to afford 2-(3-chlorophenyl)-2,2-difluoro-1-phenylethyl ((S)-3-(1-ethylcyclopropyl)-1-oxo-1-(((S)-1-oxo-3-((S)-2-oxopyrrolidin-3-yl)propan-2-yl)amino)propan-2-yl)carbamate (A258). TLC system: 5% Methanol in DCM R_{f}: 0.55 LCMS (ESI): m/z 590.3 [M+H]⁺

### Example 42: Synthesis of Compound A259

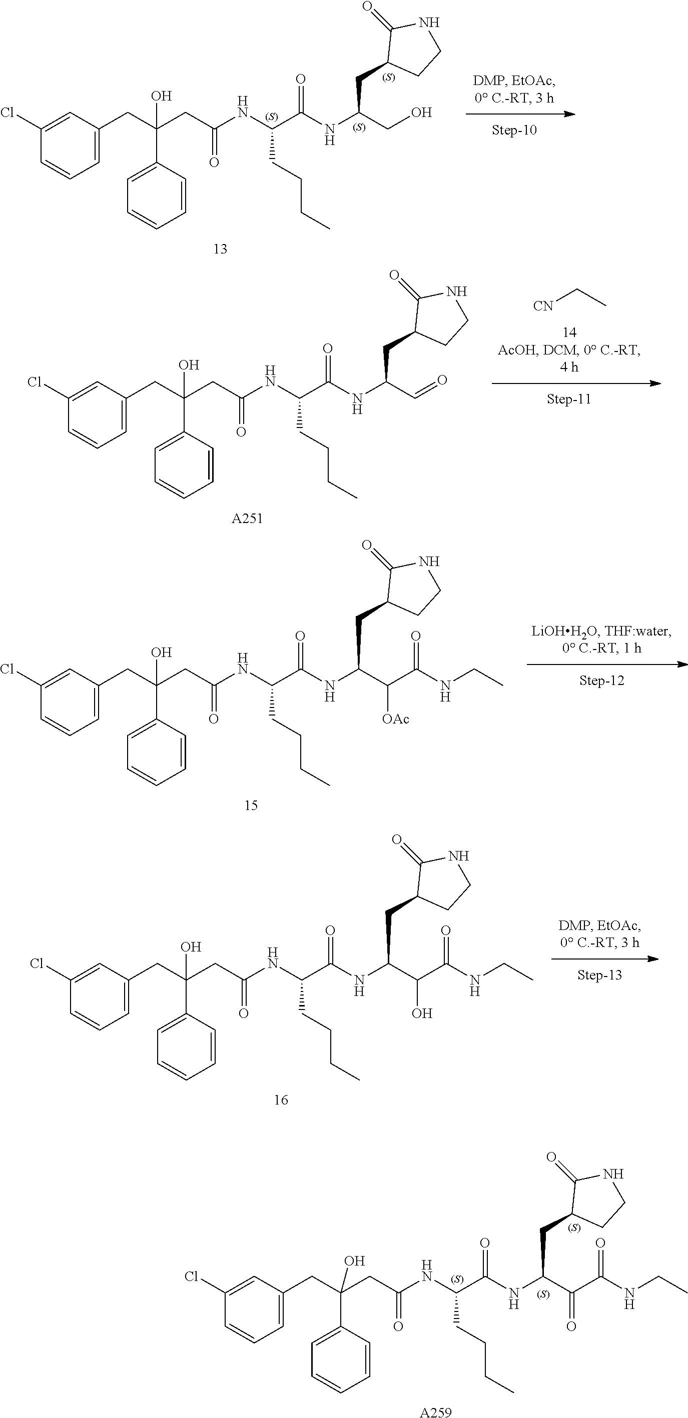

### (2S)-2-(4-(3-chlorophenyl)-3-hydroxy-3-phenylbutanamido)-N—((S)-1-oxo-3-((S)-2-oxopyrrolidin-3-yl)propan-2-yl)hexanamide (A251)

To a stirred solution of (2S)-2-(4-(3-chlorophenyl)-3-hydroxy-3-phenylbutanamido)-N—((S)-1-hydroxy-3-((S)-2-oxopyrrolidin-3-yl)propan-2-yl)hexanamide (13) (1.5 g, 2.7 mmol) in Ethyl acetate (15 mL) was added Dess-Martin periodinane (1.75 g, 6.9 mmol) at 0° C. and stirred at RT for 3 h. After completion of the reaction, reaction mixture was diluted with Ethyl acetate (30 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure to afford (2S)-2-(4-(3-chlorophenyl)-3-hydroxy-3-phenylbutanamido)-N—((S)-1-oxo-3-((S)-2-oxopyrrolidin-3-yl) propan-2-yl)hexanamide (A251). TLC system: 5% Methanol in DCM Rf: 0.4 LCMS (ESI): m/z 542.3 [M+H]⁺ TLC system: 5% Methanol in DCM Rf: 0.4 LCMS (ESI): m/z 536.7 [M+H]⁺

### ((3S)-3-((2S)-2-(4-(3-chlorophenyl)-3-hydroxy-3-phenylbutanamido)hexanamido)-1-(ethylamino)-1-oxo-4-((S)-2-oxopyrrolidin-3-yl)butan-2-yl acetate (15)

To a stirred solution of (2S)-2-(4-(3-chlorophenyl)-3-hydroxy-3-phenylbutanamido)-N—((S)-1-oxo-3-((S)-2-oxopyrrolidin-3-yl)propan-2-yl)hexanamide (A251) (750 mg, 1.38 mmol) in DCM (7.5 mL) was added isocyanoethane (14) (228 mg, 4.15 mmol) followed by Acetic acid (0.32 mL, 5.54 mmol) at 0° C. and stirred at RT for 4 h. The progress of the reaction was monitored by TLC. After 4 h, the reaction mixture was diluted with dichloromethane and washed with brine (15 mL), dried over sodium sulfate, concentrated under reduce pressure. Obtained residue was purified by reverse phase column chromatography by eluting using 60% ACN/WATER as eluent and obtained solid was triturated with pentane/diethyl ether to afford ((3S)-3-((2S)-2-(4-(3-chlorophenyl)-3-hydroxy-3-phenylbutanamido)hexanamido)-1-(ethylamino)-1-oxo-4-((S)-2-oxopyrrolidin-3-yl)butan-2-yl acetate (15). TLC system: 10% Methanol in dichloromethane Rf: 0.4 LCMS (ESI): m/z 657.31 [M+H]⁺

### (2S)-2-(4-(3-chlorophenyl)-3-hydroxy-3-phenylbutanamido)-N-((2S)-4-(ethylamino)-3-hydroxy-4-oxo-1-((S)-2-oxopyrrolidin-3-yl)butan-2-yl)hexanamide (16)

To a stirred solution of ((3S)-3-((2S)-2-(4-(3-chlorophenyl)-3-hydroxy-3-phenylbutan amido)hexanamido)-1-(ethylamino)-1-oxo-4-((S)-2-oxopyrrolidin-3-yl)butan-2-yl acetate (15) (500 mg, 0.76 mmol) in THE (3.75 mL), water (1.25 mL) was added LiOH·H₂O (48 mg, 1.14 mmol) at 0° C. and stirred at room temperature for 1 h. The progress of the reaction was monitored by TLC and LCMS. After completion of the reaction, reaction mixture was extracted with ethyl acetate (2×50 mL), combined organic layers were washed with brine solution (50 mL), dried over sodium sulfate, concentrated under reduced pressure. This residue was purified by reverse phase column chromatography by eluting using 60% ACN/WATER as eluent and obtained solid was triturated with pentane/diethyl ether to afford (2S)-2-(4-(3-chlorophenyl)-3-hydroxy-3-phenylbutanamido)-N-((2S)-4-(ethylamino)-3-hydroxy-4-oxo-1-((S)-2-oxopyrrolidin-3-yl)butan-2-yl)hexanamide (16). TLC system: 10% Methanol in dichloromethane Rf: 0.2 LCMS (ESI): m/z 615.31 [M+H]⁺

### (2S)-2-(4-(3-chlorophenyl)-3-hydroxy-3-phenylbutanamido)-N—((S)-4-(ethylamino)-3,4-dioxo-1-((S)-2-oxopyrrolidin-3-yl)butan-2-yl)hexanamide (A259)

To a stirred solution of (2S)-2-(4-(3-chlorophenyl)-3-hydroxy-3-phenylbutanamido)-N-((2S)-4-(ethylamino)-3-hydroxy-4-oxo-1-((S)-2-oxopyrrolidin-3-yl)butan-2-yl)hexanamide (16) (300 mg, 0.48 mmol) was dissolved in ethyl acetate (3.0 mL) was added Dess-Martin periodinane (310 mg, 0.732 mmol) at 0° C. and stirred at RT for 2 h. After completion of the reaction, Reaction mixture was filtered through diatomaceous earth bed and washed with ethyl acetate (10 mL). Obtained filtrate was washed with sat. sodium thiosulfate solution (3×20 mL) followed by sat. NaHCO₃ solution (3×20 mL). Organic layer was dried over anhydrous Na₂SO₄, filtered and concentrated under reduced pressure. Obtained compound was purified by trituration with n-Penatne/DEE to afford (2S)-2-(4-(3-chlorophenyl)-3-hydroxy-3-phenylbutanamido)-N—((S)-4-(ethylamino)-3,4-dioxo-1-((S)-2-oxopyrrolidin-3-yl)butan-2-yl)hexanamide (A259). TLC system: 10% Methanol in dichloromethane Rf: 0.5 LCMS (ESI): m/z 613.3 [M+H]⁺

### Example 43: Synthesis of Compound A260

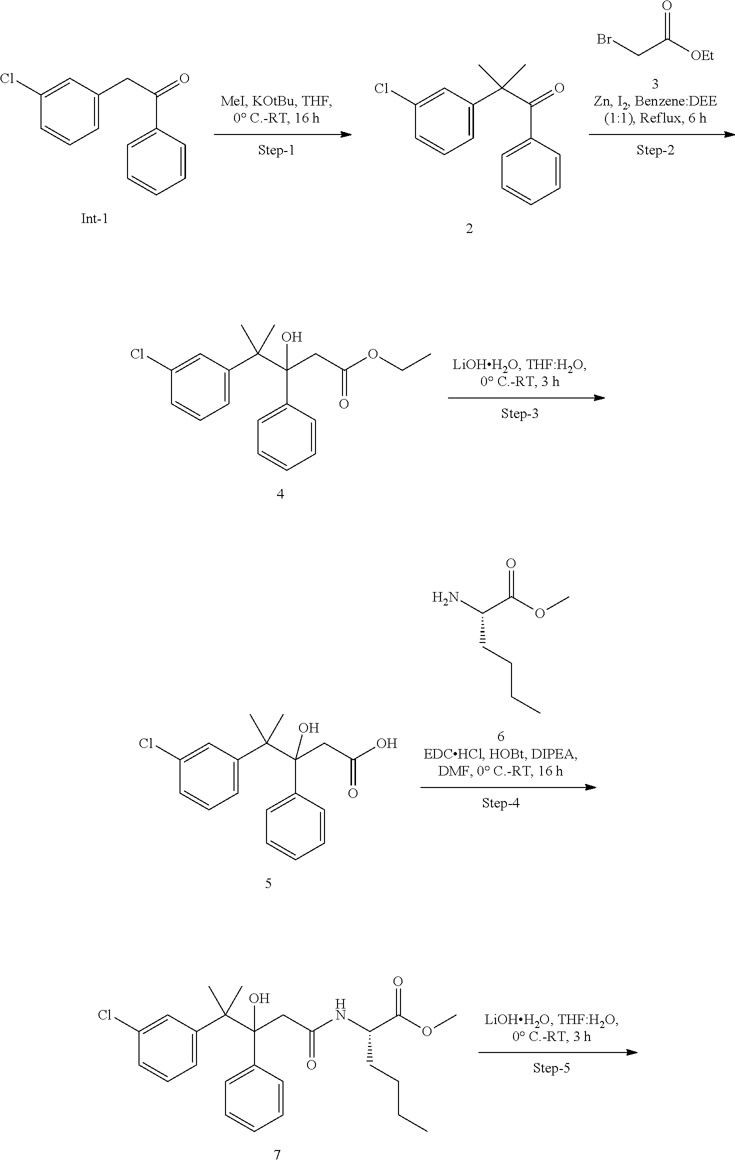

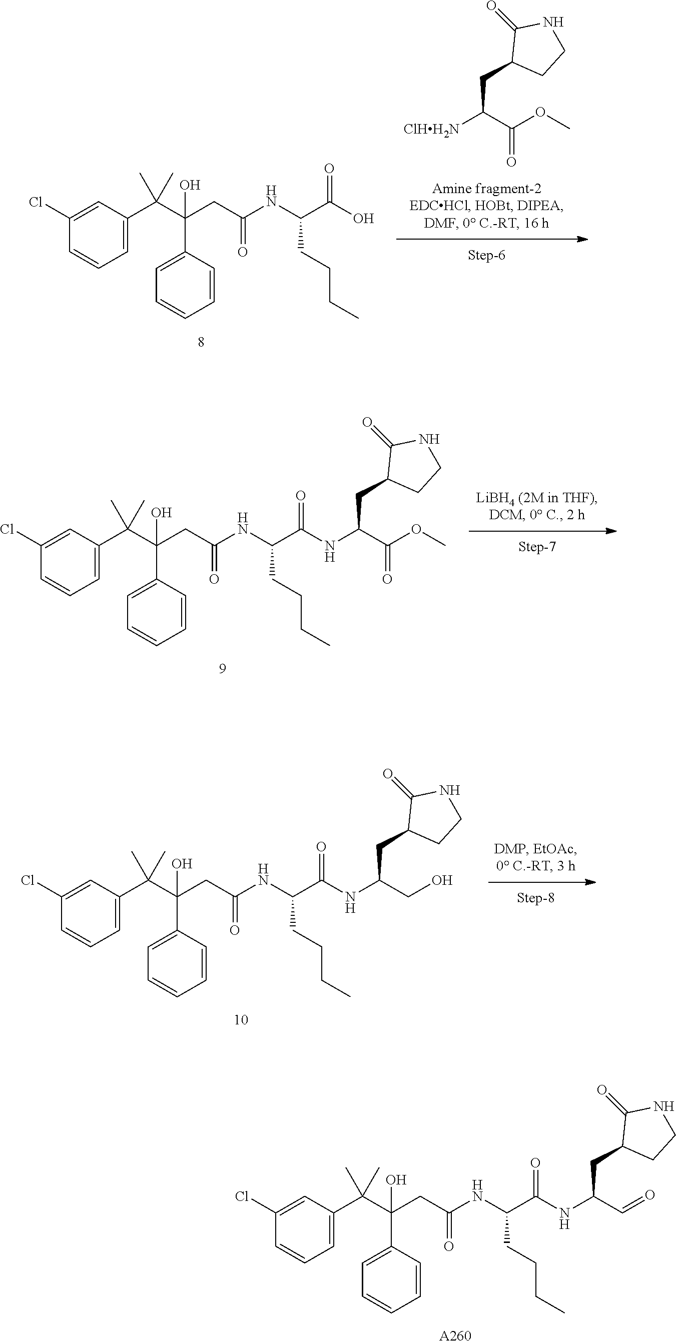

### 2-(3-chlorophenyl)-2-methyl-1-phenylpropan-1-one (2)

To a stirred solution of 2-(3-chlorophenyl)-1-phenylethan-1-one (Int-1) (7 g, 30.43 mmol) in THF (100 mL) was added potassium tert-butoxide (8.52 g, 76.07 mmol) at 0° C. and stirred at same temperature for 30 min. Then added drop wise methyl iodide (5.6 mL, 91.3 mmol) at 0° C. and stirred at RT for 16 h. Reaction progress was monitored by TLC. After completion of the reaction, reaction was quenched ice cold water and extracted with ethyl acetate (2×250 mL). Combined organic layer was washed with water (100 mL), brine solution (50 mL), dried over any. sodium sulfate and evaporated under reduced pressure to afford 2-(3-chlorophenyl)-2-methyl-1-phenylpropan-1-one (2). TLC system: 5% Ethyl acetate/Pet ether Rf: 0.5 LCMS (ESI): m/z 259.26 [M+H]⁺

### Ethyl 4-(3-chlorophenyl)-3-hydroxy-4-methyl-3-phenylpentanoate (4)

To a stirred solution of 2-(3-chlorophenyl)-2-methyl-1-phenylpropan-1-one (2) (6.0 g 23.71 mmol) in benzene:diethylether (1:1,120 mL) was added pre activated zinc (11.5 g, 177.86 mmol), cat. Iodine and ethyl bromoacetate (3) (15.7 g, 94.86 mmol) simultaneously at RT and refluxed at 80° C. for 6 h. Reaction progress was monitored by TLC.

After completion of the reaction, reaction mixture was filtered through diatomaceous earth bed and washed with ethylacetate (2×50 mL). Filtrate was quenched with 1N HCl (100 mL) and extracted with ethyl acetate (2×300 mL).

Combined organic layer was washed with water (250 mL), brine solution (100 mL), dried over anhy. Na₂SO₄ and evaporated under reduced pressure to afford ethyl 4-(3-chlorophenyl)-3-hydroxy-4-methyl-3-phenylpentanoate (4). TLC system: 20% Ethyl acetate/Pet ether Rf: 0.3 LCMS (ESI): m/z 329.40 [M−OH]⁻

### 4-(3-chlorophenyl)-3-hydroxy-4-methyl-3-phenylpentanoic acid (5)

To a stirred solution of ethyl 4-(3-chlorophenyl)-3-hydroxy-4-methyl-3-phenylpentanoate (4) (4 g, 11.53 mmol) in THE (22.5 mL) and water (5 mL) was added LiOH·H₂O (1.2 g, 28.65 mmol) at 0° C. and stirred at room temperature for 3 h. The progress of the reaction was monitored by TLC and LCMS. Excess of THE was distilled under reduced pressure, compound acidified with aq. 1N HCl solution up to pH˜2 and extracted with ethyl acetate (2×100 mL). Combined organic layer was washed with water (100 mL), brine solution (50 mL), dried over anhy. sodium sulfate and concentrated under reduced pressure to afford 4-(3-chlorophenyl)-3-hydroxy-4-methyl-3-phenylpentanoic acid (5). TLC system: 100% Ethyl acetate Rf: 0.2 LCMS (ESI): m/z 317.31 [M−H]⁺

### Methyl (2S)-2-(4-(3-chlorophenyl)-3-hydroxy-4-methyl-3-phenylpentanamido)hexanoate (7)

To a stirred solution of 4-(3-chlorophenyl)-3-hydroxy-4-methyl-3-phenylpentanoic acid (5) (5.0 g, 11.6 mmol) in DMF (100 mL) was added EDC·HCl (3.3 g, 17.4 mmol), HOBt (2.3 g, 17.4 mmol), DIPEA (4.8 mL, 34.8 mmol) and methyl (S)-2-aminohexanoate (6) (3.34 g, 15.08 mmol) at 0° C. simultaneously and stirred at room temperature for 16 h.

Reaction mixture was quenched with ice water (150 mL) and extracted with ethyl acetate (2×80 mL). Combined organic layer was washed with ice water (2×100 mL), brine solution (100 mL), dried over anhy. sodium sulfate and evaporated under reduced pressure. The residue was purified by combi-flash NP, compound eluted at 80% ethyl acetate in pet.ether to afford methyl (2S)-2-(4-(3-chlorophenyl)-3-hydroxy-4-methyl-3-phenylpentanamido)hexanoate (7). TLC system: 100% Ethyl acetate Rf: 0.4 LCMS (ESI): m/z 446.54 [M+H]⁺

### (2S)-2-(4-(3-chlorophenyl)-3-hydroxy-4-methyl-3-phenylpentanamido)hexanoic acid (8)

To a stirred solution of afford methyl (2S)-2-(4-(3-chlorophenyl)-3-hydroxy-4-methyl-3-phenylpentanamido)hexanoate (7) (6.5 g, 14.57 mmol) in THE (22.5 mL) and water (4 mL) was added LiOH·H₂O (2.44 g, 58.29 mmol) at 0° C. and stirred at room temperature for 3 h. The progress of the reaction was monitored by TLC and LCMS. Excess of THE was distilled under reduced pressure, compound acidified with aq. 1N HCl solution up to pH˜2 and extracted with ethyl 10% Methanol/DCM (2×150 mL). Combined organic layer was washed with water (100 mL), brine solution (50 mL), dried over anhy. sodium sulfate, concentrated under reduced pressure to afford (2S)-2-(4-(3-chlorophenyl)-3-hydroxy-4-methyl-3-phenylpentanamido)hexanoic acid (8). TLC system: 10% Methanol/DCM Rf: 0.3 LCMS (ESI): m/z 432.52 [M+H]⁺

### Methyl (2S)-2-((2S)-2-(4-(3-chlorophenyl)-3-hydroxy-4-methyl-3-phenyl pentanamido) hexanamido)-3-((S)-2-oxopyrrolidin-3-yl)propanoate (9)

To a stirred solution of (2S)-2-(4-(3-chlorophenyl)-3-hydroxy-4-methyl-3-phenylpentanamido)hexanoic acid (8) (2.7 g, 6.26 mmol) in DMF (40 mL) was added EDC·HCl (1.8 g, 9.39 mmol), HOBt (1.26 g, 9.39 mmol), DIPEA (3 mL, 18.78 mmol) and methyl (S)-2-amino-3-((R)-2-oxopyrrolidin-3-yl)propanoate (Amine fragment-2) (2 g, 9.39 mmol) at 0° C. simultaneously and stirred at room temperature for 16 h. After completion of the reaction, reaction mixture was quenched with ice water (100 mL) and extracted with ethyl acetate (2×80 mL). Combined organic layer was washed with brine solution (80 mL), dried over anhy. sodium sulfate and evaporated under reduced pressure. The residue was purified by combi-flash NP, compound eluted at 100% ethyl acetate afford methyl (2S)-2-((2S)-2-(4-(3-chlorophenyl)-3-hydroxy-4-methyl-3-phenylpentanamido) hexan amido)-3-((S)-2-oxopyrrolidin-3-yl)propanoate (9). TLC system: 100% Ethyl acetate Rf: 0.4 LCMS (ESI): m/z 600.30 [M+H]⁺

### (2S)-2-(4-(3-chlorophenyl)-3-hydroxy-4-methyl-3-phenylpentanamido)-N—((S)-1-hydroxy-3-((S)-2-oxopyrrolidin-3-yl)propan-2-yl)hexanamide (10)

To a stirred solution of methyl (2S)-2-((2S)-2-(4-(3-chlorophenyl)-3-hydroxy-4-methyl-3-phenylpentanamido) hexanamido)-3-((S)-2-oxopyrrolidin-3-yl)propanoate (9) (3 g, 5 mmol) in DCM (30 mL) was added 2M LiBH₄ in THE (5 mL, 10 mmol) at 0° C. and the reaction mixture stirred for 2 h at 0° C. The progress of the reaction was monitored by TLC. Reaction mixture was quenched with sat. Ammonium chloride solution (40 mL) at 0° C. and extracted with DCM (2×50 mL). Combined organic layer was washed with brine solution (30 mL), dried over Na₂SO₄ and concentrated and purified by normal phase column chromatography by using 100% Ethyl acetate to afford (2S)-2-(4-(3-chlorophenyl)-3-hydroxy-4-methyl-3-phenylpentanamido)-N—((S)-1-hydroxy-3-((S)-2-oxopyrrolidin-3-yl)propan-2-yl)hexanamide (10). TLC system: 100% ethyl acetate Rf: 0.3 LCMS (ESI): m/z 572.30 [M+H]⁺

### (2S)-2-(4-(3-chlorophenyl)-3-hydroxy-4-methyl-3-phenylpentanamido)-N—((S)-1-oxo-3-((S)-2-oxopyrrolidin-3-yl)propan-2-yl)hexanamide (A260)

To a stirred solution of (2S)-2-(4-(3-chlorophenyl)-3-hydroxy-4-methyl-3-phenylpentanamido)-N—((S)-1-hydroxy-3-((S)-2-oxopyrrolidin-3-yl)propan-2-yl)hexanamide (10) (1.0 g, 1.75 mmol) was dissolved in Ethyl acetate (15 mL) was added Dess-Martin periodinane (1.48 g, 3.5 mmol) at 0° C. and stirred at RT for 3 h. Reaction mixture was diluted with Ethyl acetate (20 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated to get compound. The compound was purified by reverse phase column afford to (2S)-2-(4-(3-chlorophenyl)-3-hydroxy-4-methyl-3-phenylpentanamido)-N—((S)-1-oxo-3-((S)-2-oxopyrrolidin-3-yl)propan-2-yl)hexanamide (A260). From that 250 mg material was purified by reverse phase column chromatography and eluted with 40% ACN/0.1% Aq. NH₄CO₃ to afford (2S)-2-(4-(3-chlorophenyl)-3-hydroxy-4-methyl-3-phenylpentanamido)-N—((S)-1-oxo-3-((S)-2-oxopyrrolidin-3-yl)propan-2-yl)hexanamide (A260). TLC system: 5% Methanol in DCM Rf: 0.4 LCMS (ESI): m/z 570.3 [M+H]⁺

### Example 44: Synthesis of Compound A261

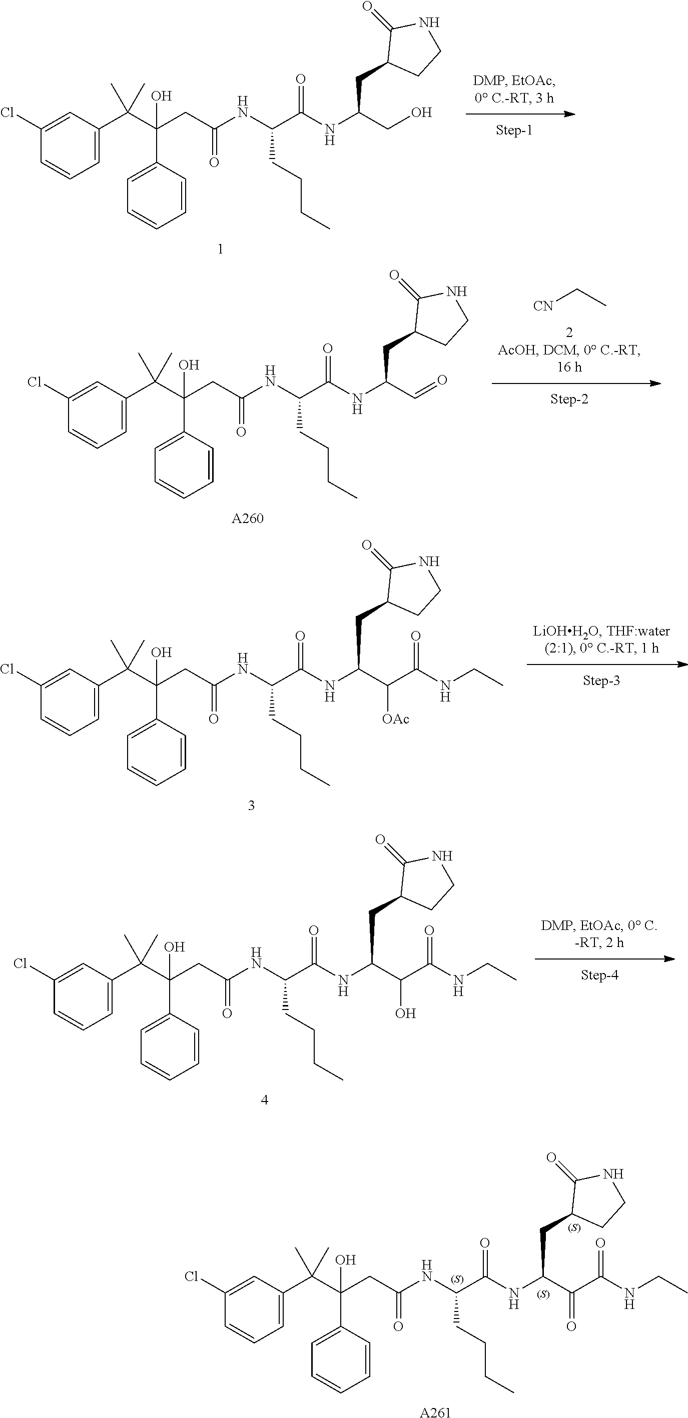

### (2S)-2-(4-(3-chlorophenyl)-3-hydroxy-4-methyl-3-phenylpentanamido)-N—((S)-1-oxo-3-((S)-2-oxopyrrolidin-3-yl)propan-2-yl)hexanamide (A260)

To a stirred solution of (2S)-2-(4-(3-chlorophenyl)-3-hydroxy-4-methyl-3-phenylpentanamido)-N—((S)-1-hydroxy-3-((S)-2-oxopyrrolidin-3-yl)propan-2-yl)hexanamide (1) (2.0 g, 3.50 mmol) was dissolved in Ethyl acetate (30 mL) was added Dess-Martin periodinane (4.4 g, 10.507 mmol) at 0° C. and stirred at RT for 3 h. Reaction mixture was diluted with Ethyl acetate (40 mL) and filtered through diatomaceous earth pad and filtrate was washed with sat. sodium thiosulfate solution (3×40 mL) followed by sat. NaHCO₃ solution (3×40 mL). Organic layer was dried over anhydrous Na₂SO₄, filtered and concentrated to get compound. The compound was purified by reverse phase column afford to 1-(3-cyclopropylphenyl)-1,1-difluoro-3-methylbutan-2-yl ((S)-4-methyl-1-oxo-1-(((S)-1-oxo-3-((S)-2-oxopyrrolidin-3-yl)propan-2-yl)amino)pentan-2-yl) carbamate (A260). TLC system: 5% Methanol in DCM Rf: 0.4 LCMS (ESI): m/z 570.3 [M+H]⁺

### (3S)-3-((2S)-2-(4-(3-chlorophenyl)-3-hydroxy-4-methyl-3-phenylpentanamido)hexanamido)-1-(ethylamino)-1-oxo-4-((S)-2-oxopyrrolidin-3-yl)butan-2-yl acetate (3)

To a stirred solution of 1-(3-cyclopropylphenyl)-1,1-difluoro-3-methylbutan-2-yl ((S)-4-methyl-1-oxo-1-(((S)-1-oxo-3-((S)-2-oxopyrrolidin-3-yl)propan-2-yl)amino)pentan-2-yl) carbamate (A260) (0.6 g, 1.05 mmol) in DCM (6 mL) was added isocyanocyclopropane (2) (86 mg, 1.57 mmol (3 ml DCM) followed by Acetic acid (0.12 mL, 2.1 mmol) at 0° C. and stirred at RT for 16 h. The progress of the reaction was monitored by TLC. After 4 h, the reaction mixture was diluted with dichloromethane and washed with brine (15 mL), dried over sodium sulfate, concentrated under reduce pressure. This residue was purified by normal phase column chromatography by eluting using 5% MeOH/DCM and obtained solid was triturated with pentane/diethyl ether to afford (3S)-1-(cyclopropylamino)-3-((2S)-2-((((1-(3-cyclopropylphenyl)-1,1-difluoro-3-methyl butan-2-yl)oxy)carbonyl)amino)-4-methylpentan amido)-1-oxo-4-((S)-2-oxopyrrolidin-3-yl) butan-2-yl acetate (3). TLC system: Ethyl acetate Rf: 0.2 LCMS (ESI): m/z 685.32 [M+H]⁺

### (2S)-2-(4-(3-chlorophenyl)-3-hydroxy-4-methyl-3-phenylpentanamido)-N-((2S)-4-(ethylamino)-3-hydroxy-4-oxo-1-((S)-2-oxopyrrolidin-3-yl)butan-2-yl)hexanamide (4)

To a stirred solution (3S)-1-(cyclopropylamino)-3-((2S)-2-((((1-(3-cyclopropylphenyl)-1,1-difluoro-3-methyl butan-2-yl)oxy)carbonyl)amino)-4-methylpentan amido)-1-oxo-4-((S)-2-oxopyrrolidin-3-yl)butan-2-yl acetate (3) (0.5 g, 0.73 mmol) in THE (4 mL), water (1 mL) was added LiOH·H₂O (61 mg, 1.46 mmol) at 0° C. and stirred at room temperature for 1 h. The progress of the reaction was monitored by TLC and LCMS. After completion of the reaction, reaction mixture was extracted with ethyl acetate (2×50 mL), combined organic layers were washed with brine solution (20 mL), dried over sodium sulfate, concentrated under reduced to afford (2S)-2-(4-(3-chlorophenyl)-3-hydroxy-4-methyl-3-phenylpentanamido)-N-((2S)-4-(ethylamino)-3-hydroxy-4-oxo-1-((S)-2-oxopyrrolidin-3-yl)butan-2-yl)hexanamide (4). TLC system: 100% Ethyl acetate Rf: 0.1 LCMS (ESI): m/z 643.46 [M+H]⁺

### (2S)-2-(4-(3-chlorophenyl)-3-hydroxy-4-methyl-3-phenylpentanamido)-N—((S)-4-(ethylamino)-3,4-dioxo-1-((S)-2-oxopyrrolidin-3-yl)butan-2-yl)hexanamide (A261)

To a stirred solution of (2S)-2-(4-(3-chlorophenyl)-3-hydroxy-4-methyl-3-phenylpentanamido)-N-((2S)-4-(ethylamino)-3-hydroxy-4-oxo-1-((S)-2-oxopyrrolidin-3-yl)butan-2-yl)hexanamide (4) (350 mg, 0.56 mmol was dissolved in ethyl acetate (5 mL) was added Dess-Martin periodinane (475 mg, 1.12 mmol) at 0° C. and stirred at RT for 2 h. Reaction mixture was filtered through diatomaceous earth bed and washed with ethyl acetate (20 mL). Filtrate was washed with sat. sodium thiosulfate solution (3×20 mL) followed by sat. NaHCO₃ solution (3×20 mL). Organic layer was dried over anhydrous Na₂SO₄, filtered and concentrated to get compound. The residue was purified by trituration with n-Penatne/DEE to afford (2S)-2-(4-(3-chlorophenyl)-3-hydroxy-4-methyl-3-phenylpentanamido)-N—((S)-4-(ethylamino)-3,4-dioxo-1-((S)-2-oxopyrrolidin-3-yl)butan-2-yl)hexanamide (A261). TLC system: 5% Methanol in dichloromethane Rf: 0.4 LCMS (ESI): m/z 641.3 [M+H]⁺

### Example 45: Synthesis of Compound A262

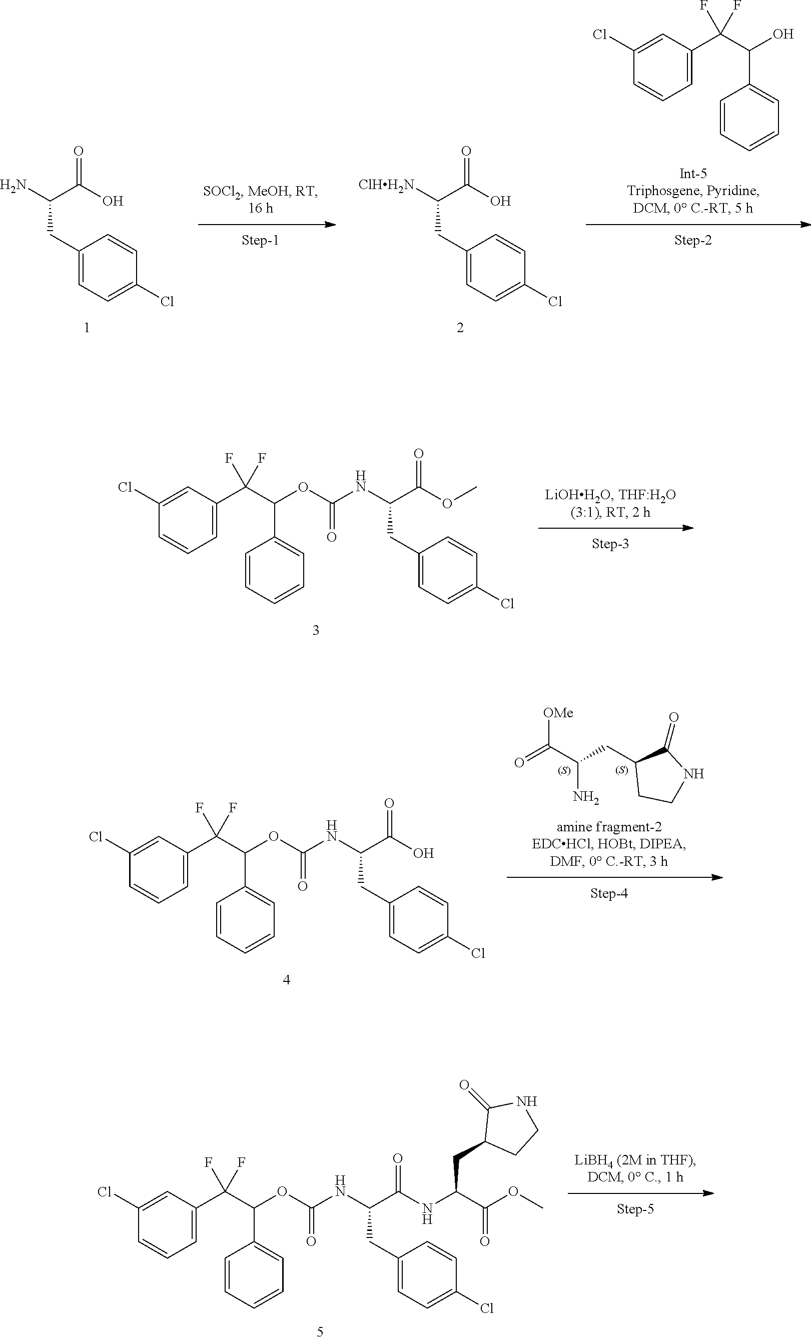

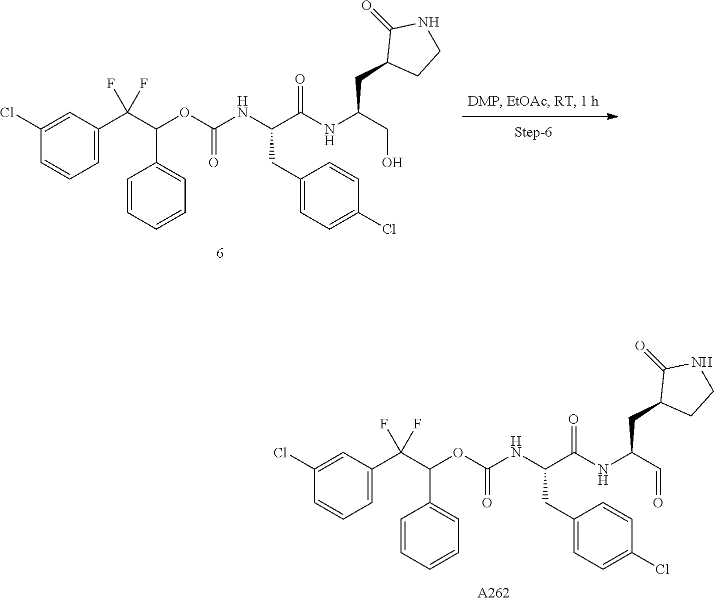 [SMILES: O=C(N[C@@H](Cc1ccc(Cl)cc1)C(=O)N[C@H](CO)C[C@@H]1CCNC1=O)OC(c1ccccc1)C(F)(F)c1cccc(Cl)c1 ; "6" ; O=C[C@H](C[C@@H]1CCNC1=O)NC(=O)[C@H](Cc1ccc(Cl)cc1)NC(=O)OC(c1ccccc1)C(F)(F)c1cccc(Cl)c1 ; "A262"]

### methyl (S)-2-amino-3-(4-chlorophenyl)propanoate hydrochloride (int-5)

To a stirred solution of (S)-2-amino-3-(4-chlorophenyl) propanoic acid (1) (9.0 g, 45 mmol) in MeOH (90 mL) was added SOCl₂ (18 mL) at 0° C. and stirred at RT for 16 h. The progress of the reaction was monitored by TLC. After completion of starting material, reaction mixture was concentrated under reduced pressure and material was washed with diethylether (50 mL) to afford methyl (S)-2-amino-3-(4-chlorophenyl) propanoate hydrochloride (2). LCMS (ESI): m/z 214.2 [M+H]⁺

### methyl (2S)-3-(4-chlorophenyl)-2-(((2-(3-chlorophenyl)-2,2-difluoro-1-phenylethoxy) carbonyl)amino)propanoate (3)

To a stirred solution of 2-(3-chlorophenyl)-2,2-difluoro-1-phenylethan-1-ol (int-7) (5 g, 18.6 mmol) in DCM (50 mL) was added pyridine (35 mL, 3 vol) followed by methyl (S)-2-amino-3-(4-chlorophenyl)propanoate hydrochloride (5.9 g, 27.98 mmol) at 0° C. To this added triphosgene (5.5 g, 18.6 mmol) slowly portion wise at 0° C. and stirred at RT 5 h. The progress of the reaction was monitored by TLC and LCMS. Reaction mixture was directly concentrated. Obtained residue was diluted with DCM and washed with 1N HCl (150 mL). Organic layer was separated, dried over anhydrous sodium sulfate and evaporated under reduced pressure. The residue was purified by combi-flash, compound eluted at 10% ethyl acetate in pet ether to afford methyl (2S)-3-(4-chlorophenyl)-2-(((2-(3-chlorophenyl)-2,2-difluoro-1-phenylethoxy)carbonyl)amino) propanoate (2). TLC system: 30% Ethyl acetate in hexane Rf: 0.55 LCMS (ESI): m/z 508.1 [M+H]⁺

### (2S)-3-(4-chlorophenyl)-2-(((2-(3-chlorophenyl)-2,2-difluoro-1-phenylethoxy)carbonyl) amino)propanoic acid (4)

To a stirred solution of methyl (2S)-3-(4-chlorophenyl)-2-(((2-(3-chlorophenyl)-2,2-difluoro-1-phenylethoxy) carbonyl) amino) propanoate (2) (4 g, 15.0 mmol) in THE (34 mL) and water (17 mL) was added LiOH·H₂O (659 mg, 15.7 mmol) at RT and stirred at room temperature for 2 h. The progress of the reaction was monitored by TLC. THE was distilled under reduced pressure, compound acidified with aq. 1N HCl solution up to pH˜2 and extracted with DCM (2×100 mL). Combined organic layer was washed with water (100 mL) brine solution (150 mL), dried over anhy. sodium sulfate and concentrated under reduced pressure to afford (2S)-3-(4-chlorophenyl)-2-(((2-(3-chlorophenyl)-2,2-difluoro-1-phenylethoxy) carbonyl) amino) propionic acid (4). TLC system: 10% Methanol in dichloromethane Rf: 0.1 LCMS (ESI): m/z 494.09 [M+H]⁺

### methyl (2S)-2-((2S)-3-(4-chlorophenyl)-2-(((2-(3-chlorophenyl)-2,2-difluoro-1-phenylethoxy)carbonyl)amino)propanamido)-3-((S)-2-oxopyrrolidin-3-yl)propanoate (5)

To a stirred solution of (2S)-3-(4-chlorophenyl)-2-(((2-(3-chlorophenyl)-2,2-difluoro-1-phenylethoxy) carbonyl) amino) propionic acid (4) (4 g, 8.11 mmol) in DMF (40 mL) was added EDC·HCl (1.8 g, 9.73 mmol), HOBt (1.0 g, 9.73 mmol), DIPEA (5.9 mL, 32.44 mmol) and methyl (S)-2-amino-3-((R)-2-oxopyrrolidin-3-yl) propanoate (Amine fragment-2′) (2.6 g, 14.6 mmol) simultaneously at 0° C. and stirred at room temperature for 3 h. After completion of the reaction (monitored by TLC), the reaction mixture was quenched with ice water (30 mL) and extracted with ethyl acetate (2×100 mL). Combined the organic layer and washed with brine solution (2×50 mL), dried over sodium sulfate and evaporated under reduced pressure. The residue was purified by combi-flash NP, compound eluted at 40% ethyl acetate to afford methyl (2S)-2-((2S)-3-(4-chlorophenyl)-2-(((2-(3-chlorophenyl)-2,2-difluoro-1-phenylethoxy)carbonyl) amino)propanamido)-3-((S)-2-oxopyrrolidin-3-yl)propanoate (5). TLC system: 5% MeOH/DCM Rf: 0.45 LCMS (ESI): m/z=662.31 [M+H]⁺

### 2-(3-chlorophenyl)-2,2-difluoro-1-phenylethyl ((S)-3-(4-chlorophenyl)-1-(((S)-1-hydroxy-3-((S)-2-oxopyrrolidin-3-yl)propan-2-yl)amino)-1-oxopropan-2-yl)carbamate (6)

To a stirred solution of methyl (2S)-2-((2S)-3-(4-chlorophenyl)-2-(((2-(3-chlorophenyl)-2,2-difluoro-1-phenylethoxy) carbonyl) amino)propanamido)-3-((S)-2-oxopyrrolidin-3-yl)propanoate (5) (3.0 g, 5.53 mmol) in DCM (30 mL) was added LiBH₄ (2M in THF, 4.5 mL, 9.07 mmol) slowly drop wise at 0° C. and stirred for 1 h at 0° C. The progress of the reaction was monitored by TLC. After completion of starting material, reaction mixture was quenched with saturated NH₄Cl solution and extracted with dichloromethane (2×100 mL). Combined organic layer was washed with water, dried over anhydrous sodium sulfate and concentrated under reduced pressure and purified by reverse phase column (C18) eluting with 50% ACN in 0.1% FA in water to afford 2-(3-chlorophenyl)-2,2-difluoro-1-phenylethyl ((S)-3-(4-chlorophenyl)-1-(((S)-1-hydroxy-3-((S)-2-oxopyrrolidin-3-yl)propan-2-yl)amino)-1-oxopropan-2-yl)carbamate (6). TLC system: 5% Methanol in dichloromethane Rf: 0.35 LCMS (ESI): m/z 634.2 [M+H]⁺

### 2-(3-chlorophenyl)-2,2-difluoro-1-phenylethyl ((S)-3-(4-chlorophenyl)-1-oxo-1-(((S)-1-oxo-3-((S)-2-oxopyrrolidin-3-yl)propan-2-yl)amino)propan-2-yl)carbamate (A262)

To a stirred solution of 2-(3-chlorophenyl)-2,2-difluoro-1-phenylethyl ((S)-3-(4-chlorophenyl)-1-(((S)-1-hydroxy-3-((S)-2-oxopyrrolidin-3-yl)propan-2-yl)amino)-1-oxopropan-2-yl)carbamate (6) (2.0 g, 3.15 mmol) in ethyl acetate (20 mL) was added Dess-Martin period inane (2.7 g, 6.3 mmol) slowly portion wise at 0° C. and stirred at RT for 1 h. The progress of the reaction was monitored by TLC and LCMS. Reaction mixture was diluted with Ethyl acetate (50 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure. Obtained material was purified by reverse phase combiflash column (C18) using 50% ACN in 0.1% aq. NH₄CO₃ solution as eluent to afford 2-(3-chlorophenyl)-2,2-difluoro-1-phenylethyl ((S)-3-(4-chlorophenyl)-1-oxo-1-(((S)-1-oxo-3-((S)-2-oxopyrrolidin-3-yl)propan-2-yl)amino)propan-2-yl)carbamate (A262). TLC system: 10% Methanol in DCM Rf: 0.55 LCMS (ESI): m/z 632.2 [M+H]⁺

### Example 46: Synthesis of Compound A263

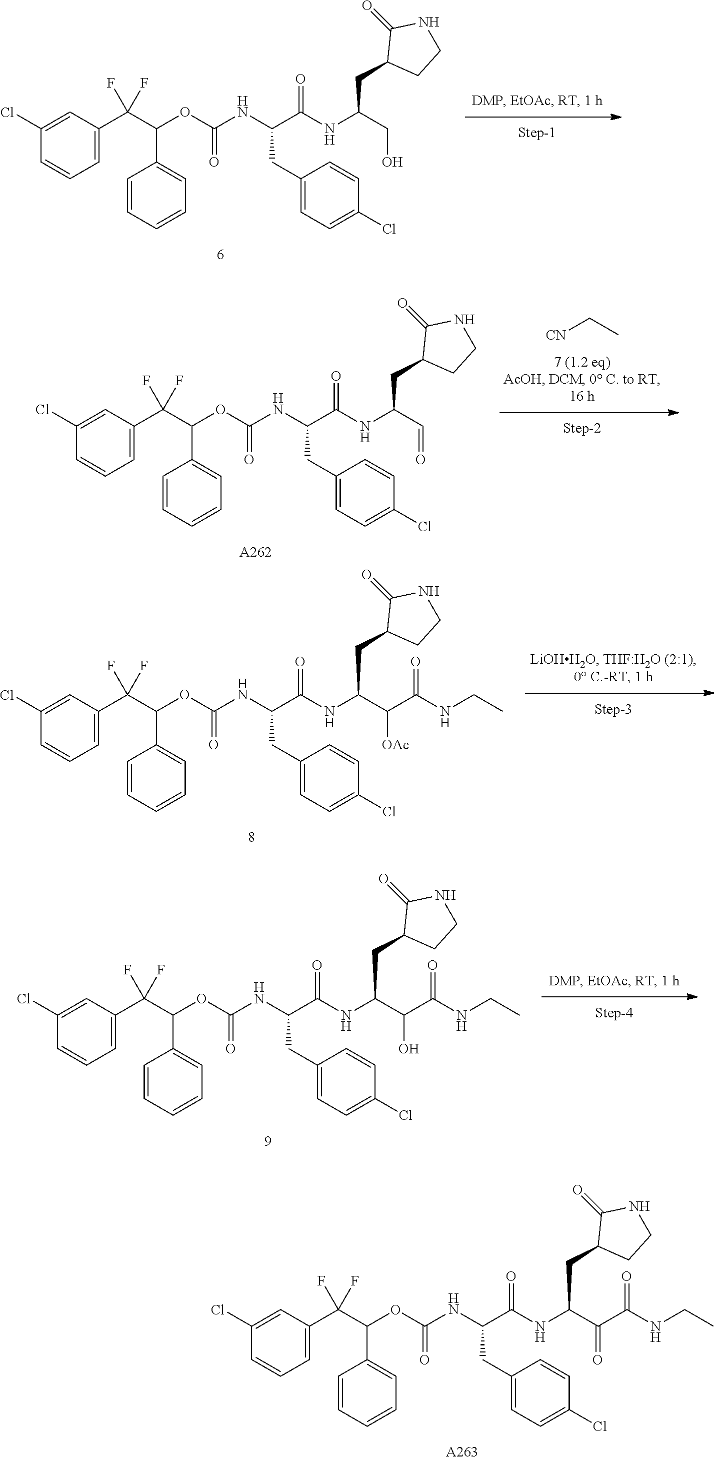

### 2-(3-chlorophenyl)-2,2-difluoro-1-phenylethyl ((S)-3-(4-chlorophenyl)-1-oxo-1-(((S)-1-oxo-3-((S)-2-oxopyrrolidin-3-yl)propan-2-yl)amino)propan-2-yl)carbamate (A262)

To a stirred solution of 2-(3-chlorophenyl)-2,2-difluoro-1-phenylethyl ((S)-3-(4-chlorophenyl)-1-(((S)-1-hydroxy-3-((S)-2-oxopyrrolidin-3-yl)propan-2-yl)amino)-1-oxopropan-2-yl)carbamate (6) (2.0 g, 3.15 mmol) in ethyl acetate (20 mL) was added Dess-Martin period inane (2.7 g, 6.3 mmol) slowly portion wise at 0° C. and stirred at RT for 1 h. The progress of the reaction was monitored by TLC and LCMS. After completion of the reaction, reaction mixture was diluted with Ethyl acetate (50 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure. Obtained material was purified by reverse phase combiflash column (C18) using 50% ACN in 0.1% aq. NH₄CO₃ solution as eluent to afford 2-(3-chlorophenyl)-2,2-difluoro-1-phenylethyl ((S)-3-(4-chlorophenyl)-1-oxo-1-(((S)-1-oxo-3-((S)-2-oxopyrrolidin-3-yl)propan-2-yl)amino) propan-2-yl)carbamate (A262). TLC system: 10% Methanol in DCM Rf: 0.55 LCMS (ESI): m/z 632.2 [M+H]⁺

### (6S,9S)-6-(4-chlorobenzyl)-1-(3-chlorophenyl)-1,1-difluoro-4,7,11-trioxo-9-(((S)-2-oxopyrrolidin-3-yl)methyl)-2-phenyl-3-oxa-5,8,12-triazatetradecan-10-yl acetate (8)

To a stirred solution of 2-(3-chlorophenyl)-2,2-difluoro-1-phenylethyl ((S)-3-(4-chlorophenyl)-1-oxo-1-(((S)-1-oxo-3-((S)-2-oxopyrrolidin-3-yl)propan-2-yl)amino)propan-2-yl)carbamate (A262) (800 mg, 1.26 mmol) in DCM (4 mL) was added isocyanoethane (7) (solution in DCM (2 vol) followed by acetic acid (0.8 mL) at 0° C. and stirred at RT for 16 h. The progress of the reaction was monitored by TLC and LCMS. After completion of starting material reaction mixture was diluted with dichloromethane and washed with sat. ammonium chloride solution (2×30 mL) followed by brine (1×20 mL). Organic layer was dried over anhydrous Na₂SO₄ and evaporated under reduced pressure. Obtained material was purified by reverse phase combi flash column (C18) using 50% ACN in 0.1% FA in water as eluent to afford (6S,9S)-6-(4-chlorobenzyl)-1-(3-chlorophenyl)-1,1-difluoro-4,7,11-trioxo-9-(((S)-2-oxopyrrolidin-3-yl)methyl)-2-phenyl-3-oxa-5,8,12-triazatetradecan-10-yl acetate (8). TLC system: 10% MeOH in DCM Rf: 0.5 LCMS (ESI): m/z 747.3 [M+H]+

### 2-(3-chlorophenyl)-2,2-difluoro-1-phenylethyl ((2S)-3-(4-chlorophenyl)-1-(((2S)-4-(ethyl amino)-3-hydroxy-4-oxo-1-((S)-2-oxopyrrolidin-3-yl)butan-2-yl)amino)-1-oxopropan-2-yl) carbamate (9)

To a stirred solution of (6S,9S)-6-(4-chlorobenzyl)-1-(3-chlorophenyl)-1,1-difluoro-4,7,11-trioxo-9-(((S)-2-oxopyrrolidin-3-yl)methyl)-2-phenyl-3-oxa-5,8,12-triazatetradecan-10-yl acetate (8) (200 mg, 0.28 mmol) in THF (3 mL) and water (1.5 mL) was added LiOH·H₂O (22.7 mg, 0.56 mmol) at 0° C. and stirred at room temperature for 1 h. The progress of the reaction was monitored by TLC and LCMS. After completion of the reaction, reaction mixture was extracted with ethyl acetate (2×50 mL). Combined organic layer was washed with brine solution (30 mL), dried over anhy. sodium sulfate and concentrated under reduced pressure to afford 2-(3-chlorophenyl)-2,2-difluoro-1-phenylethyl ((2S)-3-(4-chlorophenyl)-1-(((2S)-4-(ethylamino)-3-hydroxy-4-oxo-1-((S)-2-oxopyrrolidin-3-yl)butan-2-yl)amino)-1-oxopropan-2-yl)carbamate (9) TLC system: 10% MeOH in DCM Rf: 0.4 LCMS (ESI): m/z 705.4 [M+H]+

### 2-(3-chlorophenyl)-2,2-difluoro-1-phenylethyl ((S)-3-(4-chlorophenyl)-1-(((S)-4-(ethylamino)-3,4-dioxo-1-((S)-2-oxopyrrolidin-3-yl)butan-2-yl)amino)-1-oxopropan-2-yl)carbamate (A263)

To a stirred solution of 2-(3-chlorophenyl)-2,2-difluoro-1-phenylethyl ((2S)-3-(4-chlorophenyl)-1-(((2S)-4-(ethylamino)-3-hydroxy-4-oxo-1-((S)-2-oxopyrrolidin-3-yl)butan-2-yl)amino)-1-oxopropan-2-yl)carbamate (9) (180 mg, 0.25 mmol) in ethyl acetate (3 mL) was added Dess-Martin periodinane (216 mg, 0.511 mmol) at 0° C. and stirred at RT for 1 h. The progress of the reaction was monitored by TLC and LCMS. Reaction mixture was filtered through diatomaceous earth bed and washed with ethyl acetate (10 mL). Obtained filtrate was washed with sat. sodium thiosulfate solution (3×20 mL) followed by sat. NaHCO₃ solution (3×20 mL). Organic layer was dried over anhydrous Na₂SO₄, filtered and concentrated under reduced pressure. Obtained residue was purified by trituration with n-Penatne/DEE to afford 2-(3-chlorophenyl)-2,2-difluoro-1-phenylethyl ((S)-3-(4-chlorophenyl)-1-(((S)-4-(ethylamino)-3,4-dioxo-1-((S)-2-oxopyrrolidin-3-yl)butan-2-yl)amino)-1-oxopropan-2-yl)carbamate (A263). TLC system: 10% MeOH in DCM Rf: 0.65 LCMS (ESI): m/z 703.2 [M+H]+

### Example 47: Synthesis of Compound A266

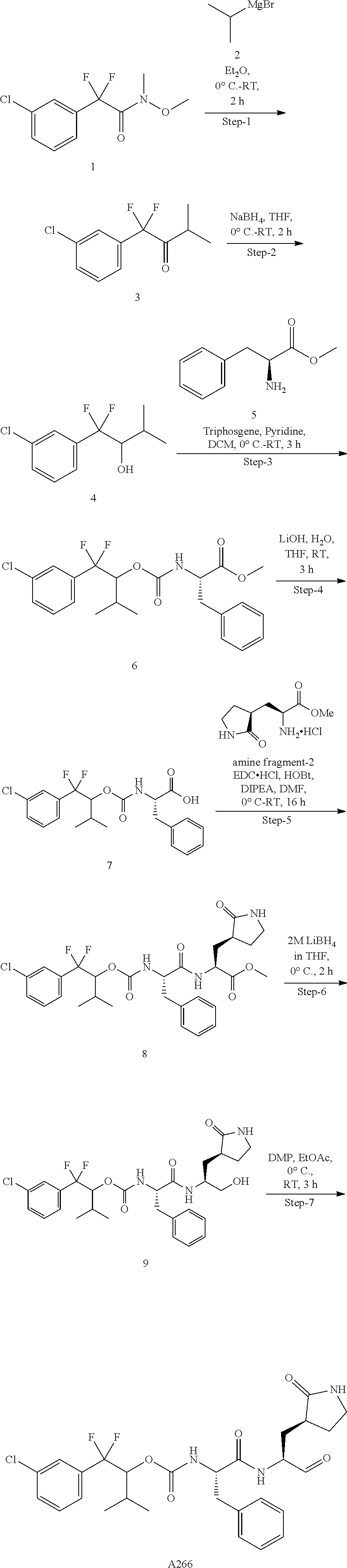

### 1-(3-Chlorophenyl)-1,1-difluoro-3-methylbutan-2-one (3)

To a stirred solution of 2-(3-chlorophenyl)-2,2-difluoro-N-methoxy-N-methylacetamide (1) (5.0 g, 20.069 mmol) in diethyl ether was added iPrMgCl (77 mL, 100.4 mmol) at 0° C. The reaction mixture was allowed to stir for 2 h at RT. Reaction progress was monitored by TLC. Reaction mixture was quenched with sat. Ammonium chloride and extracted with ethyl acetate (2×50 mL). Combined the organic layer and washed with water (1×50 mL), brine solution (1×50 mL), dried over sodium sulfate and evaporated under reduced pressure to afforded 1-(3-chlorophenyl)-1,1-difluoro-3-methylbutan-2-one (3). TLC system: 10% Ethyl acetate/Pet ether Rf: 0.5 LCMS (ESI): no ionization

### 1-(3-Chlorophenyl)-1,1-difluoro-3-methylbutan-2-ol (4)

To a stirred solution of 1-(3-chlorophenyl)-1,1-difluoro-3-methylbutan-2-one (3) (4.5 g, 19.396 mmol) in THE (45 mL) wad added sodium borohydride (2.2 g, 58.189 mmol) at 0° C. The reaction mixture was allowed to stir at RT for 2 h. After completion of the reaction by TLC, the reaction mixture was quenched with 1N Aq. HCl (50 mL) and extracted with ethyl acetate (2×30 mL). The combined organic layer was washed with water (40 mL), brine (40 mL), dried over sodium sulfate and evaporated under reduced pressure to afforded 1-(3-chlorophenyl)-1,1-difluoro-3-methylbutan-2-ol (4). TLC system: 20% Ethyl acetate/Pet ether Rf: 0.3 LCMS (ESI): no ionization

### Methyl (((1-(3-chlorophenyl)-1,1-difluoro-3-methylbutan-2-yl)oxy)carbonyl)-L-phenylalaninate (6)

To a stirred solution of 1-(3-chlorophenyl)-1,1-difluoro-3-methylbutan-2-ol (4) (3.0 g, 12.820 mmol), methyl L-phenylalaninate (5) (6.092 g, 28.204 mmol) in DCM (30 mL) was added pyridine (9 mL, 3 Vol) followed by triphosgene (8.30 g, 28.204 mmol) at 0° C. portion wise. The reaction mixture was allowed to stir at RT for 3 h. The progress of the reaction was monitored by TLC. Reaction mixture was quenched with 1N aq HCl (50 mL) and extracted with DCM (2×30 mL), dried over sodium sulfate and evaporated under reduced pressure. The material was purified by silica gel (100-200 mesh) column chromatography by eluting with 20% ethyl acetate in hexane to afford methyl (((1-(3-chlorophenyl)-1,1-difluoro-3-methylbutan-2-yl)oxy)carbonyl)-L-phenylalaninate (6). TLC system: 20% Ethyl acetate/Pet ether Rf: 0.3 LCMS (ESI): m/z 440.2 [M+H]⁺

### (((1-(3-Chlorophenyl)-1,1-difluoro-3-methylbutan-2-yl)oxy)carbonyl)-L-phenylalanine (7)

To a stirred solution of methyl (((1-(3-chlorophenyl)-1,1-difluoro-3-methylbutan-2-yl)oxy)carbonyl)-L-phenylalaninate (6) (3.8 g, 8.656 mmol) in THE (30 mL), water (10 mL) was added lithium hydroxide (623 mg, 25.968 mmol) at RT and stirred at room temperature for 3 h. The progress of the reaction was monitored by TLC. Excess of THE was distilled under reduced pressure, compound was acidified with aq. 1N HCl solution up to pH˜2 and extracted with ethyl acetate (2×50 mL). The combined organic layer was washed with water (50 mL) brine (50 mL), dried over sodium sulfate and concentrated under reduced pressure to afford (((1-(3-chlorophenyl)-1,1-difluoro-3-methylbutan-2-yl)oxy)carbonyl)-L-phenylalanine (7). TLC system: 100% Ethyl acetate Rf: 0.2 LCMS (ESI): m/z 406.2 [M−F]⁺

### Methyl (2S)-2-((2S)-2-((((1-(3-chlorophenyl)-1,1-difluoro-3-methylbutan-2-yl)oxy)carbonyl)amino)-3-phenylpropanamido)-3-((S)-2-oxopyrrolidin-3-yl)propanoate (8)

To a stirred solution of (((1-(3-chlorophenyl)-1,1-difluoro-3-methylbutan-2-yl)oxy)carbonyl)-L-phenylalanine (7) (2.5 g, 5.882 mmol) in DMF (25 mL) was added, EDC·HCl (1.694 g, 8.823 mmol), HOBt (1.191 g, 8.823 mmol), DIPEA (3.06 mL, 17.646 mmol) and methyl (S)-2-amino-3-((S)-2-oxopyrrolidin-3-yl)propanoate (Amine fragment-2) (1.567 g, 7.058 mmol) at 0° C. The reaction mixture was allowed to stir at room temperature for 16 h. Water (150 mL) was added to the reaction mixture and extracted with ethyl acetate (2×100 mL). The combined the organic layer was washed with brine (2×50 mL), dried over sodium sulfate and evaporated under reduced pressure. The material was purified by combi-flash (C-18, 0.1% ABC: acetonitrile) to afford methyl(2S)-2-((2S)-2-((((1-(3-chlorophenyl)-1,1-difluoro-3-methylbutan-2-yl)oxy) carbonyl)amino)-3-phenylpropanamido)-3-((S)-2-oxopyrrolidin-3-yl)propanoate (8). TLC system: 10% Methanol in dichloromethane Rf: 0.4 LCMS (ESI): m/z 594.3 [M+H]⁺

### 1-(3-Chlorophenyl)-1,1-difluoro-3-methylbutan-2-yl ((S)-1-(((S)-1-hydroxy-3-((S)-2-oxopyrrolidin-3-yl)propan-2-yl)amino)-1-oxo-3-phenylpropan-2-yl)carbamate (9)

To a stirred solution of methyl (2S)-2-((2S)-2-((((1-(3-chlorophenyl)-1,1-difluoro-3-methylbutan-2-yl)oxy)carbonyl)amino)-3-phenylpropanamido)-3-((S)-2-oxopyrrolidin-3-yl)propanoate (8) (2.2 g, 3.709 mmol) in THE (22 mL) was added LiBH₄ (5.5 mL, 11.129 mmol) at 0° C. and the reaction mixture stirred for 2 h at RT. The progress of the reaction was monitored by TLC. The reaction mixture was quenched with sat. Ammonium chloride solution (20 mL) and extracted with DCM (2×75 mL). Organic layer was washed with brine (30 mL), dried over Na₂SO₄ and concentrated to afford material, which was triturated with diethyl ether (25 mL) to afford 1-(3-chlorophenyl)-1,1-difluoro-3-methylbutan-2-yl ((S)-1-(((S)-1-hydroxy-3-((S)-2-oxopyrrolidin-3-yl)propan-2-yl)amino)-1-oxo-3-phenylpropan-2-yl)carbamate (9). TLC system: 10% Methanol in DCM Rf: 0.2 LCMS (ESI): m/z 566.3 [M+H]⁺

### 1-(3-Chlorophenyl)-1,1-difluoro-3-methylbutan-2-yl ((S)-1-oxo-1-(((S)-1-oxo-3-((S)-2-oxopyrrolidin-3-yl)propan-2-yl)amino)-3-phenylpropan-2-yl)carbamate (A266)

To a stirred solution of 1-(3-chlorophenyl)-1,1-difluoro-3-methylbutan-2-yl ((S)-1-(((S)-1-hydroxy-3-((S)-2-oxopyrrolidin-3-yl)propan-2-yl)amino)-1-oxo-3-phenylpropan-2-yl)carbamate (9) (500 mg, 0.884 mmol) in ethyl acetate (10 mL) was added Dess-Martin periodinane (1.1 g, 2.654 mmol) at 0° C. and stirred at RT for 3 h. The reaction mixture was diluted with ethyl acetate (20 mL) and filtered through calcite pad and washed with sat. Hypo solution (3×20 mL) followed by sat. NaHCO₃ solution (3×20 mL). Organic layer was dried over anhydrous Na₂SO₄ and concentrated to get material, which was purified by combi-flash (C-18, 0.1% ABC: acetonitrile) to obtain 1-(3-chlorophenyl)-1,1-difluoro-3-methylbutan-2-yl((S)-1-oxo-1-(((S)-1-oxo-3-((S)-2-oxopyrrolidin-3-yl)propan-2-yl)amino)-3-phenylpropan-2-yl)carbamate (A266). TLC system: 10% Methanol in DCM Rf: 0.4 LCMS (ESI): m/z 564.2 [M+H]⁺

### Example 48: Synthesis of Compounds A267 and A268

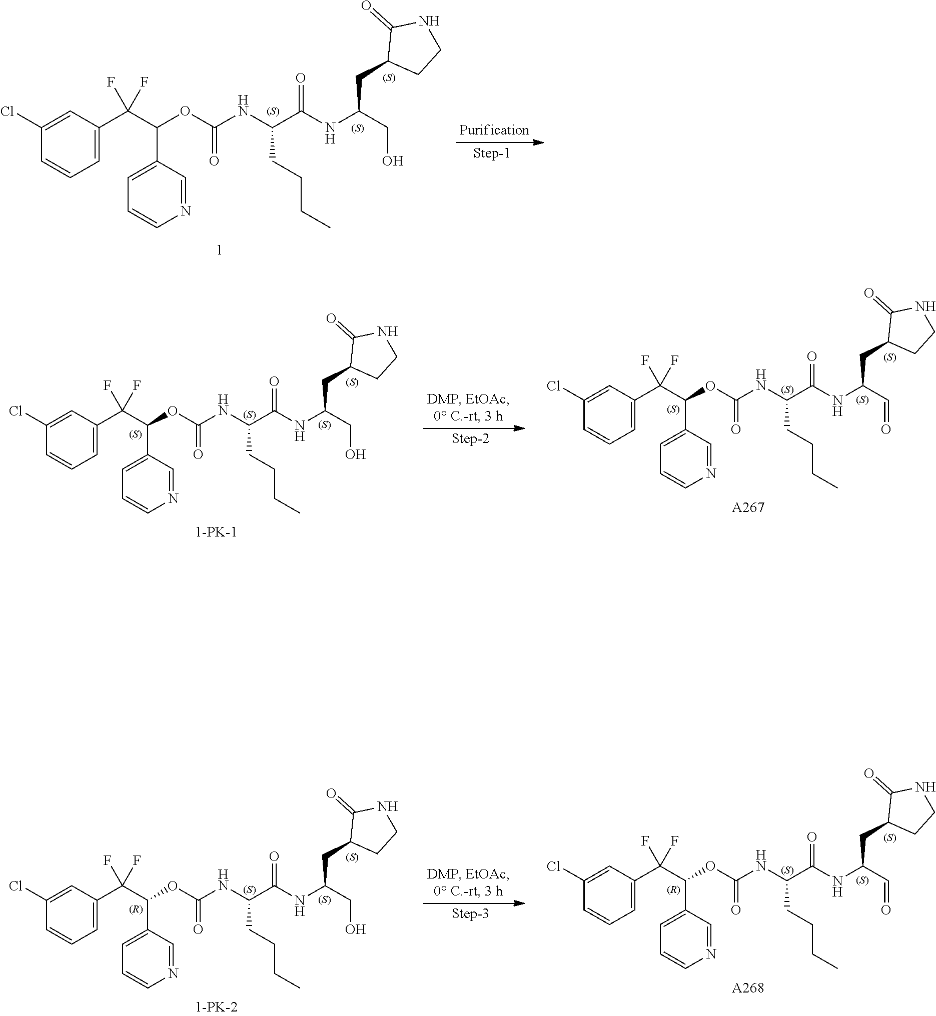

### (S)-2-(3-chlorophenyl)-2,2-difluoro-1-(pyridin-3-yl)ethyl ((S)-1-(((S)-1-hydroxy-3-((S)-2-oxopyrrolidin-3-yl)propan-2-yl)amino)-1-oxohexan-2-yl)carbamate (1-PK-1) & (R)-2-(3-chlorophenyl)-2,2-difluoro-1-(pyridin-3-yl)ethyl ((S)-1-(((S)-1-hydroxy-3-((S)-2-oxopyrrolidin-3-yl)propan-2-yl)amino)-1-oxohexan-2-yl)carbamate (1-PK-2)

800 mg of 2-(3-chlorophenyl)-2,2-difluoro-1-(pyridin-3-yl)ethyl((S)-1-(((S)-1-hydroxy-3-((S)-2-oxopyrrolidin-3-yl)propan-2-yl)amino)-1-oxohexan-2-yl)carbamate (1) was purified by combi-flash (C-18, 0.1% ABC in water: acetonitrile) to afford (S)-2-(3-chlorophenyl)-2,2-difluoro-1-(pyridin-3-yl)ethyl ((S)-1-(((S)-1-hydroxy-3-((S)-2-oxopyrrolidin-3-yl)propan-2-yl)amino)-1-oxohexan-2-yl)carbamate (1-PK-1) and (R)-2-(3-chlorophenyl)-2,2-difluoro-1-(pyridin-3-yl)ethyl ((S)-1-(((S)-1-hydroxy-3-((S)-2-oxopyrrolidin-3-yl)propan-2-yl)amino)-1-oxohexan-2-yl)carbamate (1-PK-2). LCMS (ESI): m/z 567.2 [M+H]⁺

### (S)-2-(3-chlorophenyl)-2,2-difluoro-1-(pyridin-3-yl)ethyl ((S)-1-oxo-1-(((S)-1-oxo-3-((S)-2-oxopyrrolidin-3-yl)propan-2-yl)amino)hexan-2-yl)carbamate (A267)

To a stirred solution of 2(S)-2-(3-chlorophenyl)-2,2-difluoro-1-(pyridin-3-yl)ethyl ((S)-1-(((S)-1-hydroxy-3-((S)-2-oxopyrrolidin-3-yl)propan-2-yl)amino)-1-oxohexan-2-yl)carbamate (1-PK-1) (250 mg, 0.441 mmol) in ethyl acetate (10 mL) was added Dess-Martin periodinane (563 mg, 1.32 mmol) at 0° C. and stirred at RT for 3 h. After completion of the reaction by TLC, the reaction mixture was diluted with ethyl acetate (20 mL), filtered through diatomaceous earth pad and washed with satd. Hypo solution (3×30 mL) followed by sat. NaHCO₃ solution (3×30 mL). Organic layer was dried over anhydrous Na₂SO₄, filtered and concentrated to get material which was triturated using Et₂O:pentane (1:1,15 mL) to obtain (S)-2-(3-chlorophenyl)-2,2-difluoro-1-(pyridin-3-yl)ethyl((S)-1-oxo-1-(((S)-1-oxo-3-((S)-2-oxopyrrolidin-3-yl)propan-2-yl)amino) hexan-2-yl)carbamate (A267). TLC system: 10% Methanol in DCM Rf: 0.4 LCMS (ESI): m/z 565.2 [M+H]⁺

### (R)-2-(3-chlorophenyl)-2,2-difluoro-1-(pyridin-3-yl)ethyl ((S)-1-oxo-1-(((S)-1-oxo-3-((S)-2-oxopyrrolidin-3-yl)propan-2-yl)amino)hexan-2-yl)carbamate (A268)

To a stirred solution of (R)-2-(3-chlorophenyl)-2,2-difluoro-1-(pyridin-3-yl)ethyl ((S)-1-(((S)-1-hydroxy-3-((S)-2-oxopyrrolidin-3-yl)propan-2-yl)amino)-1-oxohexan-2-yl)carbamate (1-PK-2) (250 mg, 0.441 mmol) in ethyl acetate (10 mL) was added Dess-Martin periodinane (563 mg, 1.32 mmol) at 0° C. and stirred at RT for 3 h. After completion of the reaction by TLC, the reaction mixture was diluted with ethyl acetate (20 mL), filtered through diatomaceous earth pad and washed with satd. Hypo solution (3×30 mL) followed by sat. NaHCO₃ solution (3×30 mL). Organic layer was dried over anhydrous Na₂SO₄, filtered and concentrated to get material which was triturated using Et₂O:pentane (1:1,15 mL) to obtain (R)-2-(3-chlorophenyl)-2,2-difluoro-1-(pyridin-3-yl)ethyl((S)-1-oxo-1-(((S)-1-oxo-3-((S)-2-oxopyrrolidin-3-yl)propan-2-yl)amino) hexan-2-yl)carbamate (A268). TLC system: 10% Methanol in DCM Rf: 0.4 LCMS (ESI): m/z 565.2 [M+H]⁺

### Example 49: Synthesis of Compound A269

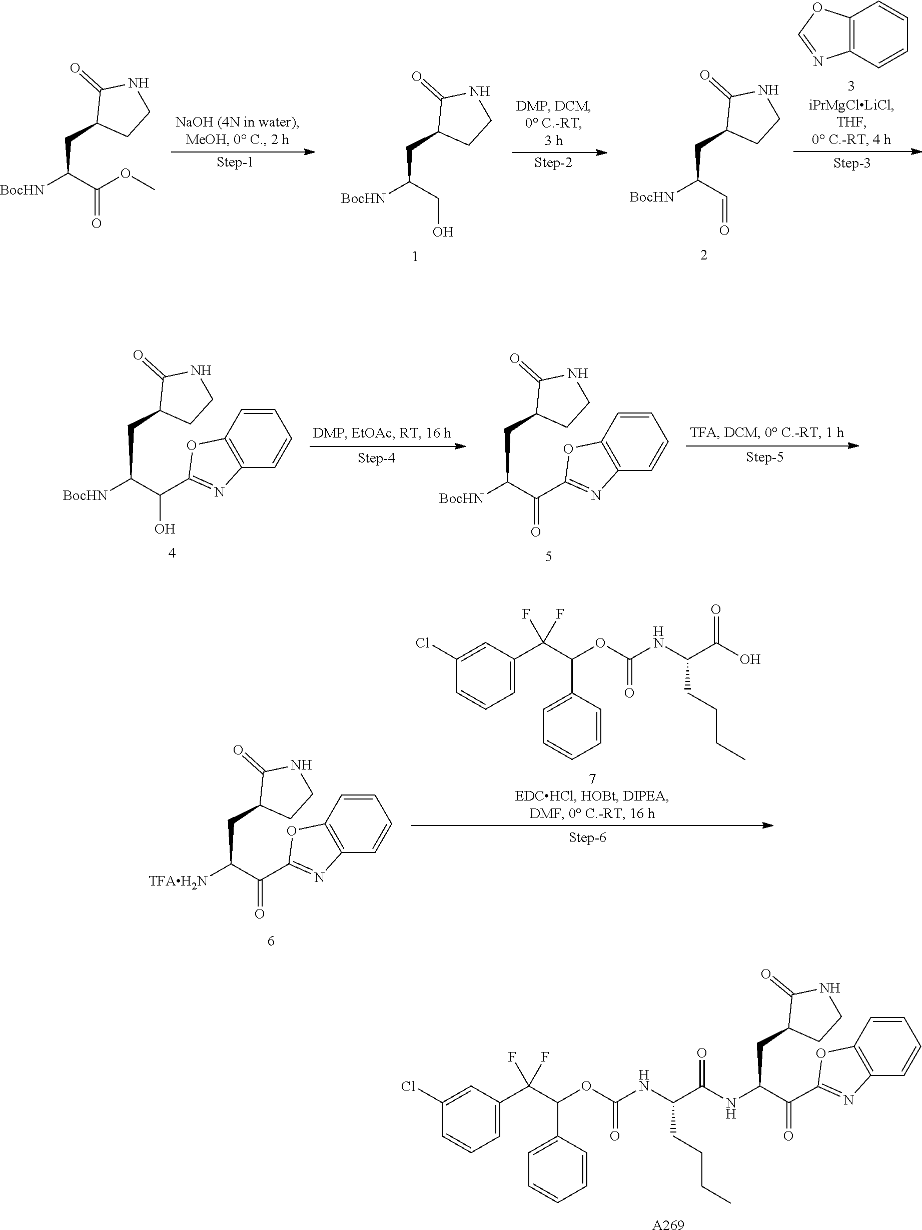

### tert-butyl ((S)-1-hydroxy-3-((S)-2-oxopyrrolidin-3-yl)propan-2-yl)carbamate (1)

To a stirred solution of methyl (S)-2-((tert-butoxycarbonyl)amino)-3-((S)-2-oxopyrrolidin-3-yl)propanoate (15 g, 52.44 mmol) in MeOH (30 mL) at −10° C. was added NaOH (8.39, 262.23 mmol, dissolved in 80 mL of water) added drop wise without raise in internal temperature. The resulting reaction mixture was stirred at −5° C. for 2 h. After completion of the reaction (monitored by TLC), the reaction mixture was evaporated under reduced pressure. Obtained residue was diluted with water (200 mL), acidified with (up to pH˜2) 1N HCl at 0° C. and extracted with EtOAc (3×200 mL). Combined organic layer was washed with brine solution (200 mL), dried over anhy. Na₂SO₄ and evaporated under reduced pressure to afford (S)-tert-butyl ((S)-1-hydroxy-3-((S)-2-oxopyrrolidin-3-yl)propan-2-yl)carbamate (1). TLC system: 10% Methanol in Dichloromethane Rf: 0.5 LCMS (ESI): m/z=271.14 (M−H)⁺

### tert-butyl ((S)-1-oxo-3-((S)-2-oxopyrrolidin-3-yl)propan-2-yl)carbamate (2)

To a stirred solution of tert-butyl ((S)-1-hydroxy-3-((S)-2-oxopyrrolidin-3-yl)propan-2-yl)carbamate (1) (14 g, 54.26 mmol) in DCM (140 mL was added DMP (34.51 g, 81.39 mmol) slowly portion wise) at 0° C. The resulting reaction mixture was stirred at RT for 3 h. After completion of the reaction (monitored by TLC and LC-MS), the reaction mixture was filtered through diatomaceous earth bed and washed with (10% M/DCM). Organic layer was washed with sat. sodium thiosulphate (300 mL) & sat. NaHCO₃ₛₒₗᵤₜᵢₒₙ (300 mL). Organic layer was dried over anhy·Na₂SO₄ and concentrated under reduced pressure to afford tert-butyl ((S)-1-oxo-3-((S)-2-oxopyrrolidin-3-yl)propan-2-yl)carbamate (3). TLC system: 10% Methanol in Dichloromethane Rf: 0.5

### tert-butyl ((2S)-1-(benzo[d]oxazol-2-yl)-1-hydroxy-3-((S)-2-oxopyrrolidin-3-yl)propan-2-yl)carbamate (4)

To a stirred solution of benzo[d]oxazole (3) (6.97 g, 58.59 mmol) in THF (30 mL) was added iPrMgCl·LiCl (1.3M in THF, 36.05 ml, 46.87 mmol) at 0° C. and stirred for 2 h. After that this solution was added to a solution of tert-butyl ((S)-1-oxo-3-((S)-2-oxopyrrolidin-3-yl)propan-2-yl)carbamate (2) (1.5 g, 5.85 mmol) in THE (7 mL) at 0° C. and stirred at 0° C. for 2 h. The progress of the reaction was monitored by TLC and LCMS. After completion of starting material, reaction mixture was quenched with sat. NH₄Cl (50 mL) and extracted with ethyl acetate (2×100 mL). Organic layer was separated, dried over anhy. Na₂SO₄ and concentrated under reduced pressure. Obtained material was purified by silica gel (100-200 mesh) column chromatography using 0-10% MeOH in DCM as eluent to afford tert-butyl ((2S)-1-(benzo[d]oxazol-2-yl)-1-hydroxy-3-((S)-2-oxopyrrolidin-3-yl)propan-2-yl)carbamate (4). TLC system: 10% Methanol in Dichloromethane Rf: 0.3 LCMS (ESI): m/z=376.61 (M+H)⁺

### tert-butyl ((S)-1-(benzo[d]oxazol-2-yl)-1-oxo-3-((S)-2-oxopyrrolidin-3-yl)propan-2-yl)carbamate (5)

To a stirred solution of tert-butyl ((2S)-1-(benzo[d]oxazol-2-yl)-1-hydroxy-3-((S)-2-oxopyrrolidin-3-yl)propan-2-yl)carbamate (4) (0.75 g, 2.00 mmol) in EtOAc (21 mL) at 0° C. was added DMP (1.69 g, 4.00 mmol). The resulting reaction mixture was stirred at RT for 16 h. After completion of the reaction (monitored by TLC and LC-MS), the reaction mixture was filtered through diatomaceous earth bed and washed with (10% M/DCM). Organic layer washed with sat. sodium thiosulphate (300 mL) & sat. NaHCO₃ (300 mL). Organic layer dried over anhy·Na2SO4 and concentrated under reduced pressure. The material was purified by reverse phase column using 0-50% (CAN/Water) to tert-butyl ((S)-1-(benzo[d]oxazol-2-yl)-1-oxo-3-((S)-2-oxopyrrolidin-3-yl) propan-2-yl)carbamate (5). TLC system: 10% Methanol in Dichloromethane Rf: 0.4 LCMS (ESI): m/z=396.17 (M+Na+H)⁺

### (S)-3-((S)-3-(benzo[d]oxazol-2-yl)-3-oxo-2-((2,2,2-trifluoroacetyl)-14-azaneyl)propyl) pyrrolidin-2-one as TFA salt (6)

To a stirred tert-butyl ((S)-1-(benzo[d]oxazol-2-yl)-1-oxo-3-((S)-2-oxopyrrolidin-3-yl)propan-2-yl)carbamate (5) (0.5 g, 1.34 mmol) in DCM (10 mL) was added TFA (3 mL) at 0° C. and stirred at RT for 1 h. The progress of the reaction was monitored by TLC and LCMS. Reaction mixture was completely distilled under reduced pressure to afford (S)-3-((S)-3-(benzo[d]oxazol-2-yl)-3-oxo-2-((2,2,2-trifluoroacetyl)-14-azaneyl)propyl)pyrrolidin-2-one as TFA salt (6). TLC system: 10% MeOH/DCM Rf: 0.2 LCMS (ESI): m/z 274.12 [M+H]⁺

### 2-(3-chlorophenyl)-2,2-difluoro-1-phenylethyl ((S)-1-(((S)-1-(benzo[d]oxazol-2-yl)-1-oxo-3-((S)-2-oxopyrrolidin-3-yl)propan-2-yl)amino)-1-oxohexan-2-yl)carbamate (A269)

To a stirred solution of (S)-3-((S)-3-(benzo[d]oxazol-2-yl)-3-oxo-2-((2,2,2-trifluoroacetyl)-14-azaneyl)propyl)pyrrolidin-2-one as TFA salt (6) (0.25 g, 0.67 mmol) in DMF (5 mL) at 0° C. was added (2S)-2-(((2-(3-chlorophenyl)-2,2-difluoro-1-phenylethoxy)carbonyl)amino)hexanoic acid (0.28 g, 0.67 mmol), HBTU (0.25 g, 0.67 mmol), DIPEA (0.37 mL, 2.02 mmol) and resulting reaction mixture was stirred at RT for 2 h. After completion of the reaction (monitored by TLC and LCMS), the reaction mixture was diluted with water (50 mL) and extracted with EtOAc (2×25 mL). The combined organic layer was washed with brine solution (50 mL), dried over anhy. Na₂SO₄ and concentrated under reduced pressure. Obtained material was purified by prep HPLC to afford 2-(3-chlorophenyl)-2,2-difluoro-1-phenylethyl ((S)-1-(((S)-1-(benzo[d]oxazol-2-yl)-1-oxo-3-((S)-2-oxopyrrolidin-3-yl)propan-2-yl)amino)-1-oxohexan-2-yl)carbamate (A269). TLC system: 80% Ethyl acetate in pet ether Rf: 0.2 LCMS (ESI): m/z 681.3 [M+H]⁺

### Example 50: Synthesis of Compounds A253 and A270

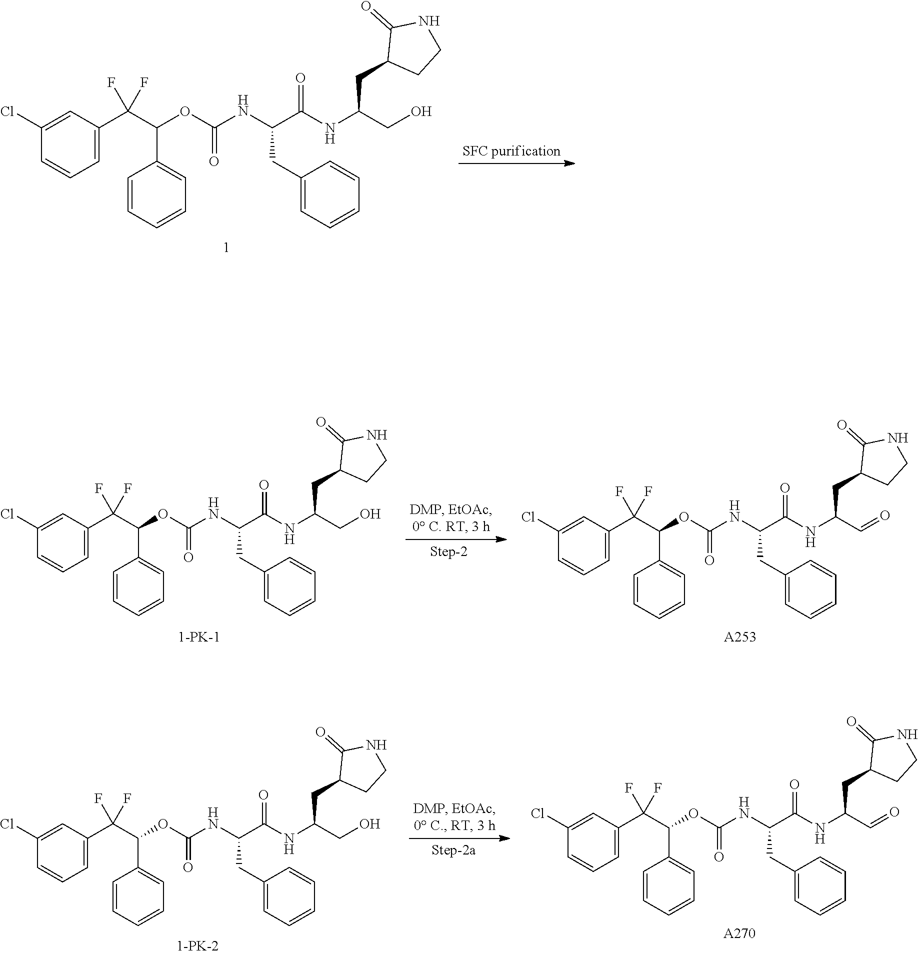

### (S)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxo-3-phenylpropan-2-yl)carbamate (1-PK-1) & (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxo-3-phenylpropan-2-yl)carbamate (1-PK-2)

400 mg of 2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxo-3-phenylpropan-2-yl)carbamate (1) was purified by Chiral SFC to afford (S)-2-(3-chlorophenyl)-2,2-difluoro-1-phenylethyl ((S)-1-(((S)-1-hydroxy-3-((S)-2-oxopyrrolidin-3-yl) propan-2-yl)amino)-1-oxo-3-phenylpropan-2-yl)carbamate (1-PK-1) and (R)-2-(3-chlorophenyl)-2,2-difluoro-1-phenylethyl((S)-1-(((S)-1-hydroxy-3-((S)-2-oxo pyrrolidin-3-yl)propan-2-yl)amino)-1-oxo-3-phenylpropan-2-yl)carbamate (1-PK-2). LCMS (ESI): m/z=600.3 [M+H]⁺

### (S)-2-(3-chlorophenyl)-2,2-difluoro-1-phenylethyl ((S)-1-oxo-1-(((S)-1-oxo-3-((S)-2-oxopyrrolidin-3-yl)propan-2-yl)amino)-3-phenylpropan-2-yl)carbamate (A253)

To a stirred solution of (S)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxo-3-phenylpropan-2-yl)carbamate (1-PK-1) (140 mg, 0.2336 mmol) in ethyl acetate (7 mL) was added Dess-Martin periodinane (298 mg, 0.7009 mmol) at 0° C. portion wise. The reaction mixture was allowed to stir at RT for 3 h. After completion of the reaction by TLC, the reaction mixture was filtered through a pad of diatomaceous earth and washed with ethyl acetate, the filtrate was washed with sat. Hypo solution (2×25 mL) followed by sat. NaHCO₃ solution (2×25 mL) and brine (2×25 mL). The organic layer was dried over anhydrous Na₂SO₄, and concentrated to get compound. The material was triturated by n-pentane/Et₂O to afford (S)-2-(3-chlorophenyl)-2,2-difluoro-1-phenylethyl ((S)-1-oxo-1-(((S)-1-oxo-3-((S)-2-oxopyrrolidin-3-yl)propan-2-yl)amino)-3-phenylpropan-2-yl)carbamate (A253). TLC system: 10% Methanol in dichloromethane Rf: 0.5 LCMS (ESI): m/z 598.2 (M+H)⁺

### (R)-2-(3-chlorophenyl)-2,2-difluoro-1-phenylethyl ((S)-1-oxo-1-(((S)-1-oxo-3-((S)-2-oxopyrrolidin-3-yl)propan-2-yl)amino)-3-phenylpropan-2-yl)carbamate (A270)

To a stirred solution of (R)-2-(3-chlorophenyl)-2,2-difluoro-1-phenylethyl ((S)-1-(((S)-1-hydroxy-3-((S)-2-oxopyrrolidin-3-yl)propan-2-yl)amino)-1-oxo-3-phenylpropan-2-yl)carbamate (1-PK-2) (130 mg, 0.217 mmol) in ethyl acetate (6.5 mL) was added Dess-Martin periodinane (276 mg, 0.651 mmol) at 0° C. portion wise. The reaction mixture was allowed to stir at RT for 3 h. After completion of the reaction by TLC, the reaction mixture was filtered through a pad of diatomaceous earth and washed with ethyl acetate, the filtrate layer was washed with sat. Hypo solution (2×25 mL) followed by sat. NaHCO₃ solution (2×25 mL) and brine (2×25 mL). The organic layer was dried over anhydrous Na₂SO₄, and concentrated to get compound. The material was triturated by n-Pentane/Et₂O to afford (R)-2-(3-chlorophenyl)-2,2-difluoro-1-phenylethyl ((S)-1-oxo-1-(((S)-1-oxo-3-((S)-2-oxopyrrolidin-3-yl)propan-2-yl)amino)-3-phenylpropan-2-yl)carbamate (A270). TLC system: 10% Methanol in dichloromethane Rf: 0.5 LCMS (ESI): m/z 598.3 (M+H)⁺

### Example 51: Synthesis of Compound A271

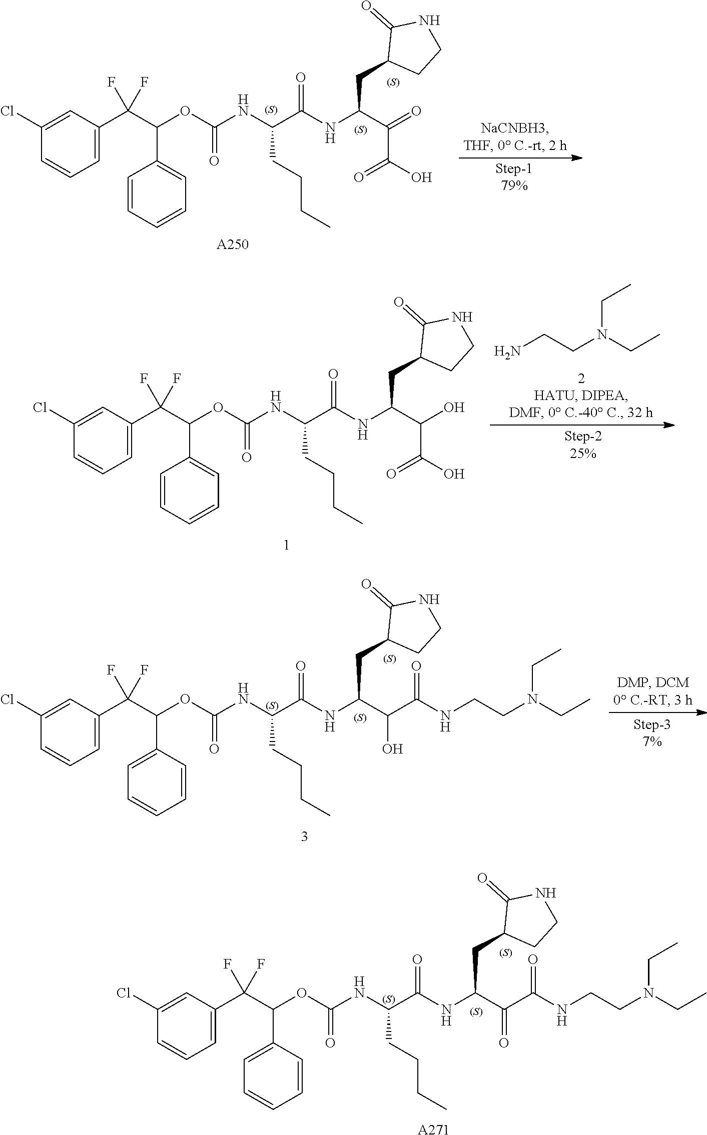

### (3S)-3-((2S)-2-(((2-(3-chlorophenyl)-2,2-difluoro-1-phenylethoxy) carbonyl)amino) hexanamido)-2-hydroxy-4-((S)-2-oxopyrrolidin-3-yl)butanoic acid (1)

To a stirred solution of (3S)-3-((2S)-2-(((2-(3-chlorophenyl)-2,2-difluoro-1-phenyl ethoxy)carbonyl)amino)hexanamido)-2-oxo-4-((S)-2-oxopyrrolidin-3-yl)butanoic acid (A250, 1 g, 1.644 mmol) in THE (30 mL) was added NaCNBH₃ (2.466 mmol) at 0° C. and stirred at RT for 2 h The progress of the reaction was monitored by TLC. The reaction mixture was quenched with satd. NH₄Cl solution (20 mL) and extracted with Ethyl acetate (2×25 mL). The combined organic layer was washed with water (25 mL), dried over sodium sulfate and evaporated under reduced pressure to get the product. This material was triturated with n-pentane (15 mL) to afford (3S)-3-((2S)-2-(((2-(3-chlorophenyl)-2,2-difluoro-1-phenylethoxy) carbonyl) amino) hexanamido)-2-hydroxy-4-((S)-2-oxopyrrolidin-3-yl) butanoic acid (1). TLC system: 15% Methanol in dichloromethane Rf: 0.3 LCMS (ESI): m/z 610.3 [M+H]⁺

### 2-(3-chlorophenyl)-2,2-difluoro-1-phenylethyl((2S)-1-(((2S)-4-((2-(diethylamino)ethyl) amino)-3-hydroxy-4-oxo-1-((S)-2-oxopyrrolidin-3-yl)butan-2-yl)amino)-1-oxohexan-2-yl)carbamate (3)

To a stirred solution of (3S)-3-((2S)-2-(((2-(3-chlorophenyl)-2,2-difluoro-1-phenyl ethoxy) carbonyl) amino) hexanamido)-2-hydroxy-4-((S)-2-oxopyrrolidin-3-yl) butanoic acid (1) (500 mg, 0.819 mmol) in DMF (5 mL) was added HATU (467 mg 1.229 mmol), DIPEA (0.43 mL, 2.45 mmol) and N, N-diethylethane-1,2-diamine (2) (115 mg, 0.983 mmol) at 0° C. and stirred at 40° C. for 32 h. The progress of the reaction was monitored by TLC and LC-MS. The reaction mixture was diluted with ethyl acetate (10 mL) and washed with sat NaHCO₃ solution (3×10 mL) followed by brine (1×10 mL). Organic layer was dried over anhydrous Na₂SO₄ and concentrated to get material, which was purified by reverse phase chromatography by using 0.1% ABC in water in acetonitrile to afford 2-(3-chlorophenyl)-2,2-difluoro-1-phenylethyl ((2S)-1-(((2S)-4-((2-(diethylamino)ethyl)amino)-3-hydroxy-4-oxo-1-((S)-2-oxopyrrolidin-3-yl)butan-2-yl)amino)-1-oxohexan-2-yl)carbamate (3). TLC system: 15% Methanol in dichloromethane Rf: 0.2 LCMS (ESI): m/z 708.3 [M+H]⁺

### 2-(3-chlorophenyl)-2,2-difluoro-1-phenylethyl((S)-1-(((S)-4-((2-(diethylamino)ethyl) amino)-3,4-dioxo-1-((S)-2-oxopyrrolidin-3-yl)butan-2-yl)amino)-1-oxohexan-2-yl)carbamate-A271

To a stirred solution of 2-(3-chlorophenyl)-2,2-difluoro-1-phenylethyl ((2S)-1-(((2S)-4-((2-(diethyl amino) ethyl) amino)-3-hydroxy-4-oxo-1-((S)-2-oxopyrrolidin-3-yl) butan-2-yl) amino)-1-oxohexan-2-yl) carbamate (3) (150 mg 0.212 mmol) in DCM (7.5 mL) was added Dess-Martin periodinane (134 mg, 0.318 mmol) at 0° C. and stirred at RT for 3 h. After completion of the reaction by TLC and LC-MS, the reaction mixture was diluted with DCM (10 mL) and washed with sat. Na₂S₂O₃ (2×20 mL), satd. NaHCO₃ solution (2×20 mL). and brine (2×20 mL). Organic layer was dried over anhydrous Na₂SO₄ and concentrated to get material, which was purified prep. HPLC to afford 2-(3-chlorophenyl)-2,2-difluoro-1-phenylethyl ((S)-1-(((S)-4-((2-(diethylamino)ethyl)amino)-3,4-dioxo-1-((S)-2-oxopyrrolidin-3-yl)butan-2-yl)amino)-1-oxohexan-2-yl)carbamate (A271).

Prep-HPLC condition: Column/dimensions X BRIDGE PHENYL-(19*250, 5 μm) Mobile phase A: 10 MM ABC in water; Mobile Phase B: 5 MM ABC IN ACN/MeOH (80:20) Gradient (Time/% B) 0/30, 1/30, 15/65, 19/65, 19.05/98, 24/98, 24.05/30, 27/30. Flow rate: 16 mL/min; solubility: Acetonitrile+THF+Water. TLC system: 10% Methanol in dichloromethane Rf: 0.4 LC-MS (ESI): m/z 706.4 [M+H]⁺

### Example 52: Synthesis of Compounds A272 and A273

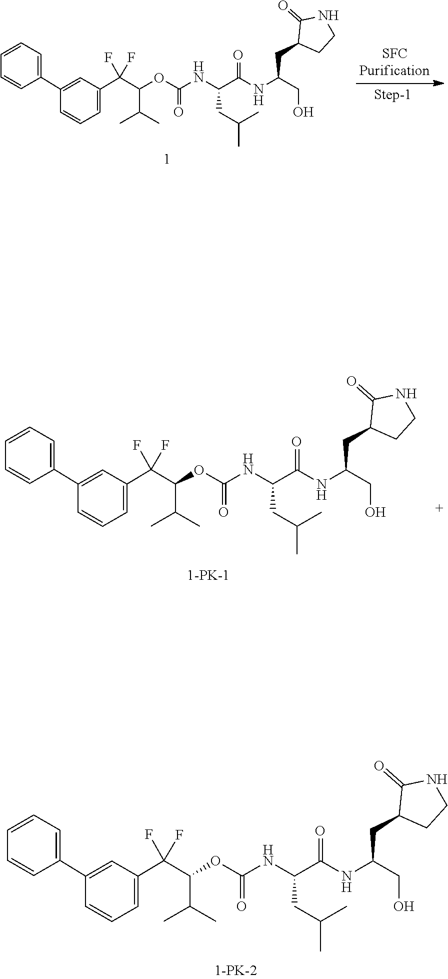 [SMILES: CC(C)C[C@H](NC(=O)OC(C(C)C)C(F)(F)c1cccc(-c2ccccc2)c1)C(=O)N[C@H](CO)C[C@@H]1CCNC1=O ; CC(C)C[C@H](NC(=O)O[C@@H](C(C)C)C(F)(F)c1cccc(-c2ccccc2)c1)C(=O)N[C@H](CO)C[C@@H]1CCNC1=O ; CC(C)C[C@H](NC(=O)O[C@H](C(C)C)C(F)(F)c1cccc(-c2ccccc2)c1)C(=O)N[C@H](CO)C[C@@H]1CCNC1=O]

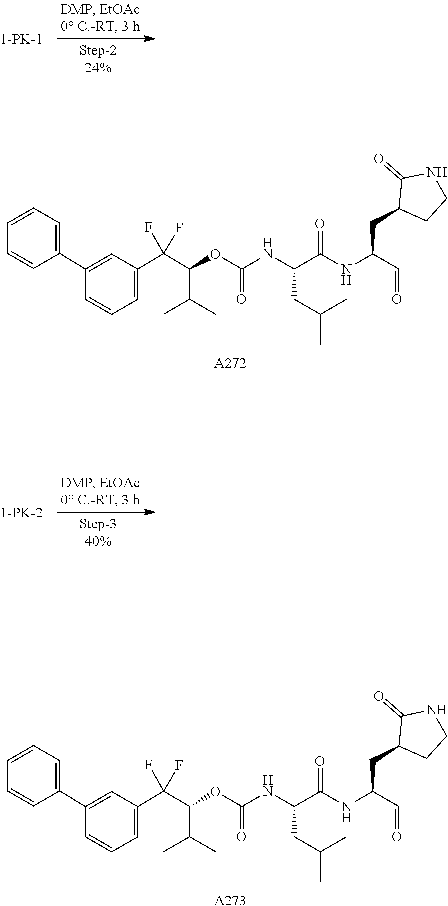 [SMILES: CC(C)C[C@H](NC(=O)O[C@@H](C(C)C)C(F)(F)c1cccc(-c2ccccc2)c1)C(=O)N[C@H](C=O)C[C@@H]1CCNC1=O ; "A272" ; CC(C)C[C@H](NC(=O)O[C@H](C(C)C)C(F)(F)c1cccc(-c2ccccc2)c1)C(=O)N[C@H](C=O)C[C@@H]1CCNC1=O ; "A273"]

### (S)-1-([1,1′-biphenyl]-3-yl)-1,1-difluoro-3-methylbutan-2-yl((S)-1-(((S)-1-hydroxy-3-((S)-2-oxopyrrolidin-3-yl)propan-2-yl)amino)-4-methyl-1-oxopentan-2-yl)carbamate (1-PK-1) & (R)-1-([1,1′-biphenyl]-3-yl)-1,1-difluoro-3-methylbutan-2-yl((S)-1-(((S)-1-hydroxy-3-((S)-2-oxopyrrolidin-3-yl)propan-2-yl)amino)-4-methyl-1-oxopentan-2-yl)carbamate (1-PK-2)

2.5 g (1) of 1-([1,1′-biphenyl]-3-yl)-1,1-difluoro-3-methylbutan-2-yl ((S)-1-(((S)-1-hydroxy-3-((S)-2-oxopyrrolidin-3-yl)propan-2-yl)amino)-4-methyl-1-oxopentan-2-yl)carbamate was purified by chiral SFC to afford (S)-1-([1,1′-biphenyl]-3-yl)-1,1-difluoro-3-methylbutan-2-yl((S)-1-(((S)-1-hydroxy-3-((S)-2-oxo pyrrolidin-3-yl)propan-2-yl)amino)-4-methyl-1-oxopentan-2-yl)carbamate (1-PK-1) (1.2 g, 2.0917 mmol, 50% yield) & (R)-1-([1,1′-biphenyl]-3-yl)-1,1-difluoro-3-methylbutan-2-yl ((S)-1-(((S)-1-hydroxy-3-((S)-2-oxopyrrolidin-3-yl)propan-2-yl)amino)-4-methyl-1-oxopentan-2-yl)carbamate (1-PK-2). LCMS (ESI): m/z 574.36 [M+H]⁺

### (S)-1-([1,1′-biphenyl]-3-yl)-1,1-difluoro-3-methylbutan-2-yl ((S)-4-methyl-1-oxo-1-(((S)-1-oxo-3-((S)-2-oxopyrrolidin-3-yl)propan-2-yl)amino)pentan-2-yl)carbamate (A272)

To a stirred solution of (S)-1-([1,1′-biphenyl]-3-yl)-1,1-difluoro-3-methylbutan-2-yl ((S)-1-(((S)-1-hydroxy-3-((S)-2-oxopyrrolidin-3-yl)propan-2-yl)amino)-4-methyl-1-oxopentan-2-yl)carbamate (1-PK-1) (500 mg, 0.8715 mmol) in ethyl acetate (10 mL) was added Dess-Martin periodinane (739.34 mg, 1.743 mmol) at 0° C. and stirred at RT for 3 h.

The progress of the reaction was monitored by TLC and LC-MS. After reaction, filtered through diatomaceous earth bed and washed with ethyl acetate and filtrate was washed with sat. NaHCO₃ solution (3×50 mL) followed by sat. Hypo solution (3×50 mL). Organic layer was dried over anhydrous Na₂SO₄, filtered and concentrated to get material. The obtained compound was purified using combi-flash (C-18, 0.1% ammonium bicarbonate in water: acetonitrile to afford (S)-1-([1,1′-biphenyl]-3-yl)-1,1-difluoro-3-methylbutan-2-yl ((S)-4-methyl-1-oxo-1-(((S)-1-oxo-3-((S)-2-oxopyrrolidin-3-yl) propan-2-yl) amino) pentan-2-yl) carbamate (A272). TLC system: 10% MeOH in DCM Rf: 0.4 LCMS (ESI): m/z 572.19 [M+H]⁺

### (R)-1-([1,1′-biphenyl]-3-yl)-1,1-difluoro-3-methylbutan-2-yl ((S)-4-methyl-1-oxo-1-(((S)-1-oxo-3-((S)-2-oxopyrrolidin-3-yl)propan-2-yl)amino)pentan-2-yl)carbamate (A273)

To a stirred solution (R)-1-([1,1′-biphenyl]-3-yl)-1,1-difluoro-3-methylbutan-2-yl ((S)-1-(((S)-1-hydroxy-3-((S)-2-oxopyrrolidin-3-yl)propan-2-yl)amino)-4-methyl-1-oxopentan-2-yl)carbamate (1-PK-2) (500 mg, 0.8715 mmol) in ethyl acetate (10 mL) was added Dess-Martin periodinane (739.34 mg, 1.743 mmol) at 0° C. and stirred at RT for 3 h. The progress of the reaction was monitored by TLC and LC-MS. The suspension was filtered through diatomaceous earth bed and washed with ethyl acetate and the organic layer was washed with sat. NaHCO₃ solution (3×50 mL) followed by sat. Hypo solution (3×50 mL). Organic layer was dried over anhydrous Na₂SO₄, filtered and concentrated to get material. The obtained compound was purified combi-flash (C-18, 0.1% ammonium bicarbonate in water: acetonitrile to afford (R)-1-([1,1′-biphenyl]-3-yl)-1,1-difluoro-3-methylbutan-2-yl((S)-4-methyl-1-oxo-1-(((S)-1-oxo-3-((S)-2-oxo pyrrolidin-3-yl)propan-2-yl)amino)pentan-2-yl)carbamate (A273). TLC system: 10% MeOH in DCM Rf: 0.4 LCMS (ESI): m/z 572.3 [M+H]⁺

### Example 53: Synthesis of Compound A274

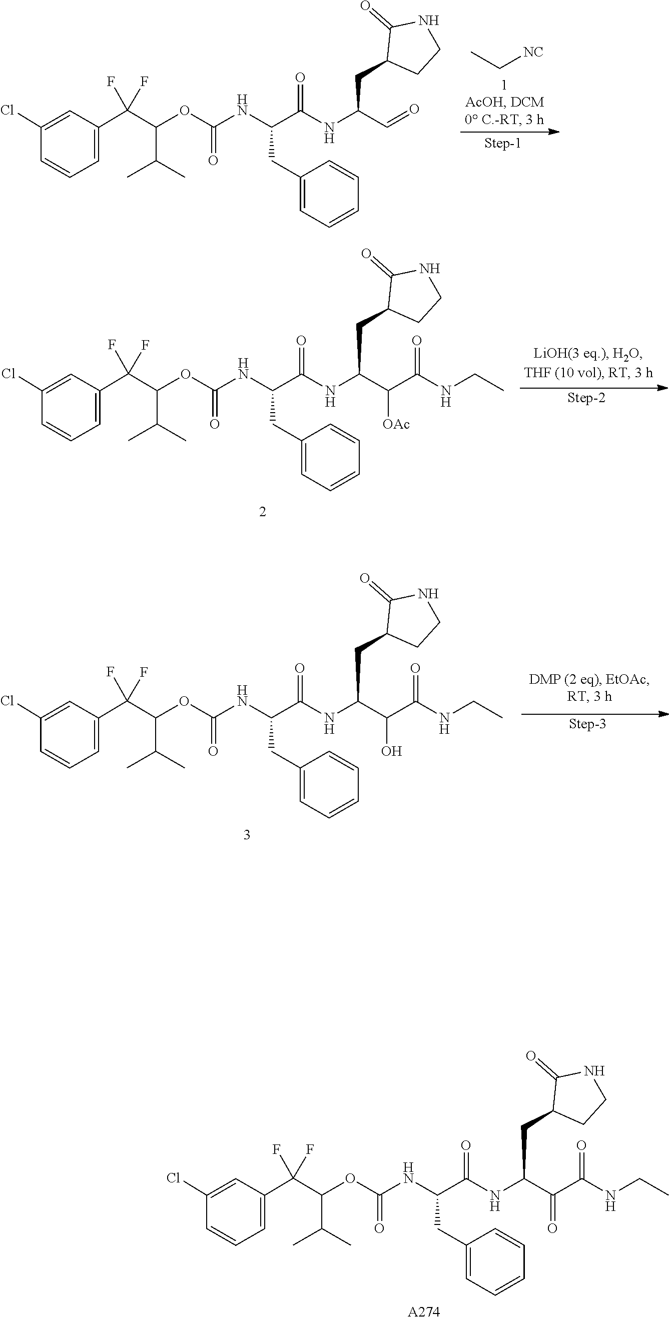

### (6S,9S)-9-Benzyl-13-((3-chlorophenyl)difluoromethyl)-14-methyl-4,8,11-trioxo-6-(((S)-2-oxopyrrolidin-3-yl)methyl)-12-oxa-3,7,10-triazapentadecane-5-yl acetate (2)

To a stirred solution of 1-(3-chlorophenyl)-1,1-difluoro-3-methylbutan-2-yl ((S)-1-oxo-1-(((S)-1-oxo-3-((S)-2-oxopyrrolidin-3-yl)propan-2-yl)amino)-3-phenylpropan-2-yl)carbamate (A266) (150 mg, 0.2487 mmol) in DCM (5 mL) was added Ethyl isocyanide (1) (7 mL, 4V) sequentially at 0° C. followed by Acetic acid (0.04 mL, 0.7462 mmol) at 0° C. and stirred at RT for 3 h. The progress of the reaction was monitored by TLC and LC-MS. Reaction mixture was diluted with dichloromethane and washed with sat. NaHCO₃ (3×10 mL) solution followed by brine (1×10 mL). The organic layer was dried over anhydrous Na₂SO₄ and evaporated under reduced pressure to afford (6S,9S)-9-benzyl-13-((3-chlorophenyl)difluoromethyl)-14-methyl-4,8,11-trioxo-6-(((S)-2-oxopyrrolidin-3-yl)methyl)-12-oxa-3,7,10-triazapentadecane-5-yl acetate (2). TLC system: 10% Methanol in dichloromethane Rf: 0.5 LCMS (ESI): m/z 679.3 [M+H]⁺

### 1-(3-Chlorophenyl)-1,1-difluoro-3-methylbutan-2-yl((2S)-1-(((2S)-4-(ethylamino)-3-hydroxy-4-oxo-1-((S)-2-oxopyrrolidin-3-yl)butan-2-yl)amino)-1-oxo-3-phenylpropan-2-yl)carbamate (3)

To a stirred solution of (6S,9S)-9-benzyl-13-((3-chlorophenyl)difluoromethyl)-14-methyl-4,8,11-trioxo-6-(((S)-2-oxopyrrolidin-3-yl)methyl)-12-oxa-3,7,10-triazapentadecan-5-yl acetate (2) 160 mg, 0.2228 mmol) in THE (1.6 mL), water (1.6 mL) was added lithium hydroxide (28 mg, 0.6685 mmol) at 0° C. and stirred at room temperature for 3 h. The progress of the reaction was monitored by TLC. Solvent was evaporated completely under reduced pressure and acidified with aq. 1N HCl solution up to pH˜2 and extracted with ethyl acetate (2×10 mL). The combined organic layer was dried over sodium sulfate, concentrated under reduced pressure to afford 1-(3-chlorophenyl)-1,1-difluoro-3-methylbutan-2-yl ((2S)-1-(((2S)-4-(ethylamino)-3-hydroxy-4-oxo-1-((S)-2-oxopyrrolidin-3-yl)butan-2-yl)amino)-1-oxo-3-phenylpropan-2-yl)carbamate (3). TLC system: 10% Methanol in dichloromethane Rf: 0.4 LCMS (ESI): m/z 637.3 (M+H)⁺

### 1-(3-Chlorophenyl)-1,1-difluoro-3-methylbutan-2-yl ((S)-1-(((S)-4-(ethylamino)-3,4-dioxo-1-((S)-2-oxopyrrolidin-3-yl)butan-2-yl)amino)-1-oxo-3-phenylpropan-2-yl)carbamate (A274)

To a stirred solution of 1-(3-chlorophenyl)-1,1-difluoro-3-methylbutan-2-yl ((2S)-1-(((2S)-4-(ethylamino)-3-hydroxy-4-oxo-1-((S)-2-oxopyrrolidin-3-yl)butan-2-yl)amino)-1-oxo-3-phenylpropan-2-yl)carbamate (3) (150 mg, 0.2218 mmol) in ethyl acetate (7.5 mL) was added Dess-Martin periodinane (282 mg, 0.6656 mmol) at 0° C. The reaction mixture was allowed to stir at RT for 3 h. After completion of the reaction by TLC, the suspension was filtered through diatomaceous earth bed and washed with ethyl acetate (20 mL). The filtrate layer was washed with sat. Hypo solution (3×25 mL) followed by sat. NaHCO₃ solution (3×25 mL) and brine (3×25 mL). Organic layer was dried over anhydrous Na₂SO₄, and concentrated to get material, which was triturated using Et₂O: pentane to afford 1-(3-chlorophenyl)-1,1-difluoro-3-methylbutan-2-yl ((S)-1-(((S)-4-(ethylamino)-3,4-dioxo-1-((S)-2-oxopyrrolidin-3-yl)butan-2-yl)amino)-1-oxo-3-phenylpropan-2-yl)carbamate (A274). TLC system: 10% Methanol in dichloromethane Rf: 0.4 LCMS (ESI): m/z 635.3 (M+H)⁺

### Example 54: Synthesis of Compounds A275 and A265

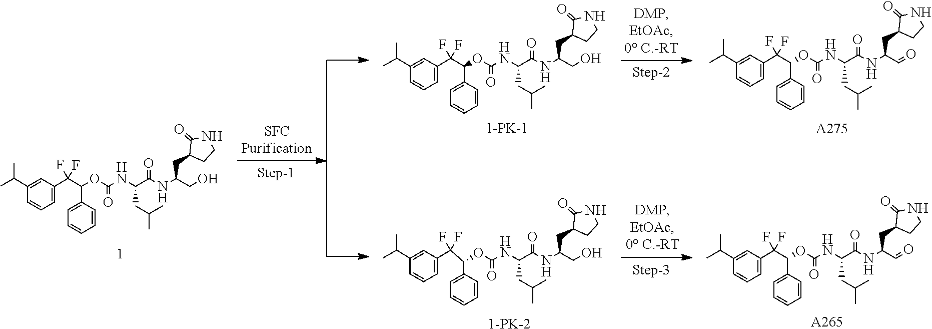

### (S)-2,2-difluoro-2-(3-isopropylphenyl)-1-phenylethyl ((S)-1-(((S)-1-hydroxy-3-((S)-2-oxo pyrrolidin-3-yl) propan-2-yl)amino)-4-methyl-1-oxopentan-2-yl)carbamate (1-PK-1) and (R)-2,2-difluoro-2-(3-isopropyl phenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (1-PK-2)

0.6 g of 2,2-difluoro-2-(3-isopropylphenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (1) was purified by chiral SFC to afford (S)-2,2-difluoro-2-(3-isopropylphenyl)-1-phenylethyl ((S)-1-(((S)-1-hydroxy-3-((S)-2-oxopyrrolidin-3-yl)propan-2-yl)amino)-4-methyl-1-oxopentan-2-yl) carbamate (1-PK-1) and (R)-2,2-difluoro-2-(3-isopropylphenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (1-PK-2). LCMS (ESI): m/z 574.7 [M+H]⁺

### (S)-2,2-difluoro-2-(3-isopropylphenyl)-1-phenylethyl ((S)-4-methyl-1-oxo-1-(((S)-1-oxo-3-((S)-2-oxopyrrolidin-3-yl)propan-2-yl)amino)pentan-2-yl)carbamate (A275)

To a stirred solution of (S)-2,2-difluoro-2-(3-isopropylphenyl)-1-phenylethyl ((S)-1-(((S)-1-hydroxy-3-((S)-2-oxopyrrolidin-3-yl)propan-2-yl)amino)-4-methyl-1-oxopentan-2-yl) carbamate (1-PK-1) (180 mg, 0.31 mmol) in ethyl acetate (2 mL) was added Dess-Martin periodinane (0.2 g, 0.465 mmol) slowly portion wise at 0° C. and stirred at RT for 2 h. The progress of the reaction was monitored by TLC and LCMS. Reaction mixture was diluted with ethyl acetate (5 mL) and filtered through a pad of diatomaceous earth. Obtained filtrate was washed with sat. NaHCO₃ solution (3×20 mL) followed by sat. Hypo solution (3×20 mL). Organic layer was separated, dried over anhydrous Na₂SO₄, filtered and concentrated under reduced pressure. Obtained material was purified by reverse phase combi flash column (C-18) and 55% of ACN in 1% Ammonium bicarbonate in water to afford (S)-2,2-difluoro-2-(3-isopropylphenyl)-1-phenylethyl ((S)-4-methyl-1-oxo-1-(((S)-1-oxo-3-((S)-2-oxopyrrolidin-3-yl)propan-2-yl)amino)pentan-2-yl)carbamate (A275). TLC system: 10% Methanol in DCM Rf: 0.55 LCMS (ESI): m/z 572.4 [M+H]⁺

### (R)-2,2-difluoro-2-(3-isopropyl phenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (1-PK-2)

To a stirred solution of (R)-2,2-difluoro-2-(3-isopropylphenyl)-1-phenylethyl ((S)-1-(((S)-1-hydroxy-3-((S)-2-oxopyrrolidin-3-yl)propan-2-yl)amino)-4-methyl-1-oxopentan-2-yl) carbamate (1-PK-2) (170 mg, 0.29 mmol) in ethyl acetate (2 mL) was added Dess-Martin periodinane (189 g, 0.445 mmol) slowly portion wise at 0° C. and stirred at RT for 2 h. The progress of the reaction was monitored by TLC and LCMS. Reaction mixture was diluted with ethyl acetate (5 mL) and filtered through a pad of diatomaceous earth. Obtained filtrate was washed with sat. NaHCO₃ solution (3×20 mL) followed by sat. Hypo solution (3×20 mL). Organic layer was separated, dried over anhydrous Na₂SO₄, filtered and concentrated under reduced pressure. Obtained material was purified by reverse phase combi flash column (C-18) and 55% of ACN in 1% Ammonium bicarbonate in water to afford (R)-2,2-difluoro-2-(3-isopropylphenyl)-1-phenylethyl ((S)-4-methyl-1-oxo-1-(((S)-1-oxo-3-((S)-2-oxopyrrolidin-3-yl)propan-2-yl)amino)pentan-2-yl) carbamate (A265). TLC system: 10% Methanol in DCM Rf: 0.55 LCMS (ESI): m/z 572.3 [M+H]⁺

### Example 55: Synthesis of Compounds A276 and A277

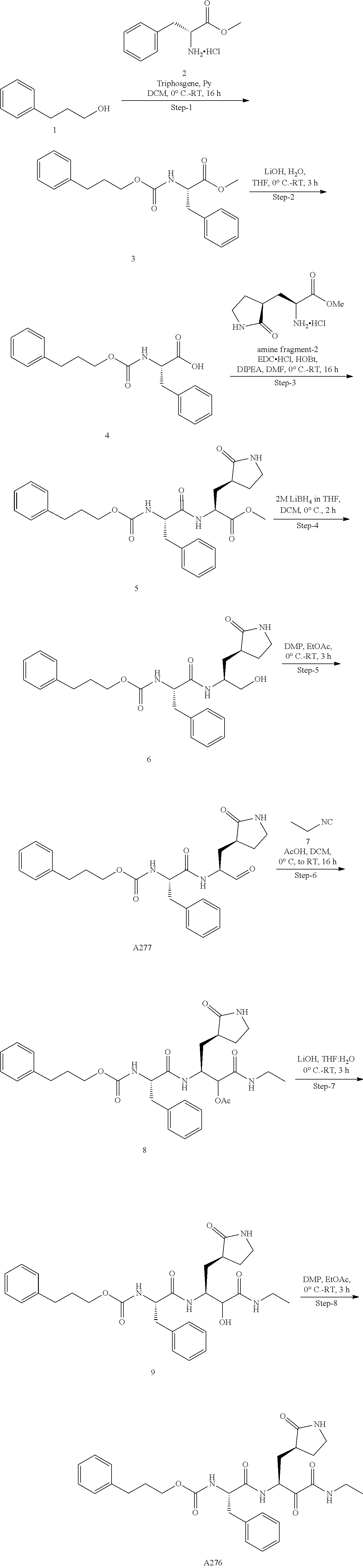

### Methyl ((3-phenylpropoxy) carbonyl)-L-phenylalaninate (3)

To a stirred solution of 3-phenylpropan-1-ol (1) (5 g, 36.713 mmol), methyl S-phenylalaninate hydrochloride (8.5 g, 44.055 mmol) in DCM (60 mL) was added pyridine (15 mL, 3 vol) followed by triphosgene (5.4 g, 18.35 mmol) at 0° C. with portion wise and stirred at RT for 16 h. The progress of the reaction was monitored by TLC. Reaction mixture was quenched with 1N aq. HCl (100 mL) then extracted with DCM (2×75 mL), washed with brine (1×50 mL), organic layer was dried over sodium sulfate and evaporated under reduced pressure. The material was purified by silica gel (230-400 mesh) column chromatography by eluting with 5% ethyl acetate in hexane to afford methyl ((3-phenylpropoxy) carbonyl)-L-phenylalaninate (3). TLC system: 10% Ethyl acetate/Pet ether R_{f}: 0.5 LCMS (ESI): m/z 342.17 [M+H]⁺

### ((3-Phenylpropoxy) carbonyl)-L-phenylalanine (4)

To a stirred solution of methyl ((3-phenylpropoxy) carbonyl)-L-phenylalaninate (3) (5 g, 14.66 mmol) in THF (40 mL), water (10 mL) was added lithium hydroxide (701 mg, 29.29 mmol) at 0° C. and stirred at room temperature for 2 h. The progress of the reaction was monitored by TLC. THF was distilled under reduced pressure, compound was acidified with aq. 1N HCl solution up to pH˜2 and extracted with ethyl acetate (2×50 mL), combined organic layer was washed with water (50 mL) brine (50 mL), dried over sodium sulfate, concentrated under reduced pressure to afford ((3-phenylpropoxy) carbonyl)-L-phenylalanine (4). TLC system: 100% EtOAc Rf: 0.5 LCMS (ESI): m/z 328.43 [M+H]⁺

### Methyl (S)-3-((S)-2-oxopyrrolidin-3-yl)-2-((S)-3-phenyl-2-(((3-phenylpropoxy) carbonyl) amino) propanamido) propanoate (5)

To a stirred solution of ((3-phenylpropoxy) carbonyl)-L-phenylalanine (4) (2 g, 6.109 mmol) in DMF (10 mL) was added EDC·HCl (1.75 g, 9.163 mmol), HOBt (1.23 g, 9.163 mmol), DIPEA (3.1 mL, 18.327 mmol) and methyl (S)-2-amino-3-((S)-2-oxopyrrolidin-3-yl)propanoate hydrochloride (amine fragment-2) (1.63 g, 7.330 mmol) at 0° C. simultaneously and stirred at room temperature for 16 h. Reaction mixture was quenched with ice water (100 mL), extracted with ethyl acetate (2×50 mL). The combined organic layer was washed with ice water (2×30 mL), dried over sodium sulphate and evaporated under reduced pressure to afford compound. The residue was purified by silica-gel (230-400 mesh) column chromatography to afford methyl (S)-3-((S)-2-oxopyrrolidin-3-yl)-2-((S)-3-phenyl-2-(((3-phenylpropoxy) carbonyl) amino) propanamido) propanoate (5). TLC system: 10% Methanol in dichloromethane Rf: 0.4 LCMS (ESI): m/z=496.29 [M+H]⁺

### 3-Phenylpropyl ((S)-1-(((S)-1-hydroxy-3-((S)-2-oxopyrrolidin-3-yl) propan-2-yl) amino)-1-oxo-3-phenylpropan-2-yl) carbamate (6)

To a stirred solution of methyl (S)-3-((S)-2-oxopyrrolidin-3-yl)-2-((S)-3-phenyl-2-(((3-phenylpropoxy) carbonyl) amino) propanamido) propanoate (5) (1 g, 2.017 mmol,) in dichloromethane (15 mL) was added 2M LiBH₄ in THE (2.0 mL, 4.035 mmol) was added at 0° C. and stirred for 2 h. The progress of the reaction was monitored by TLC. Reaction mixture was quenched with saturated NH₄Cl solution (25 mL) and extracted with dichloromethane (2×30 mL), dried over sodium sulfate, concentrated under reduced pressure to afford 3-phenylpropyl ((S)-1-(((S)-1-hydroxy-3-((S)-2-oxopyrrolidin-3-yl) propan-2-yl) amino)-1-oxo-3-phenylpropan-2-yl) carbamate (6). TLC system: 10% Methanol in dichloromethane Rf: 0.3 LCMS (ESI): m/z 468.3 [M+H]⁺

### 3-Phenylpropyl ((S)-1-oxo-1-(((S)-1-oxo-3-((S)-2-oxopyrrolidin-3-yl) propan-2-yl) amino)-3-phenylpropan-2-yl) carbamate (A277)

To a stirred solution of 3-phenylpropyl ((S)-1-(((S)-1-hydroxy-3-((S)-2-oxopyrrolidin-3-yl) propan-2-yl) amino)-1-oxo-3-phenylpropan-2-yl) carbamate (6) (100 mg, 2.147 mmol) in ethyl acetate (5 mL) was added Dess-Martin periodinane (140 mg, 2.577 mmol) at 0° C. and stirred at RT for 3 h. The progress of the reaction was monitored by TLC and LCMS. Reaction mixture was diluted with ethyl acetate (15 mL) and filtered through diatomaceous earth bed. Filtrate was washed with sat. NaHCO₃ solution (3×20 mL) followed by sat. Hypo solution (3×20 mL). Organic layer was dried over anhydrous Na₂SO₄, filtered and concentrated to get material, this material was purified by reverse phase chromatography by using 0.1% ABC in water: acetonitrile to afford 3-phenylpropyl ((S)-1-oxo-1-(((S)-1-oxo-3-((S)-2-oxopyrrolidin-3-yl) propan-2-yl) amino)-3-phenylpropan-2-yl) carbamate (A277). TLC system: 10% Methanol in DCM R_{f}: 0.5 LCMS (ESI): m/z 466.3 [M+H]⁺

### (6S,9S)-9-benzyl-4,8,11-trioxo-6-(((S)-2-oxopyrrolidin-3-yl) methyl)-15-phenyl-12-oxa-3,7,10-triazapentadecan-5-yl acetate (8)

To a stirred solution of 3-phenylpropyl ((S)-1-oxo-1-(((S)-1-oxo-3-((S)-2-oxopyrrolidin-3-yl) propan-2-yl) amino)-3-phenylpropan-2-yl) carbamate (A277) (250 mg, 0.536 mmol) in DCM (4 mL) was added AcOH (0.2 mL, 1.610 mmol), isocyanocyclopropane (60 mg, 1.072 mmol) at 0° C. and stirred at RT for 16 h. The progress of the reaction was monitored by TLC. Reaction mixture was diluted with dichloromethane (10 mL) and washed with water (15 mL) and brine (15 mL). Organic layer was dried over anhydrous Na₂SO₄ and evaporated under reduced pressure to afford (6S,9S)-9-benzyl-4,8,11-trioxo-6-(((S)-2-oxopyrrolidin-3-yl) methyl)-15-phenyl-12-oxa-3,7,10-triazapentadecan-5-yl acetate (8). TLC system: 10% MeOH in DCM Rf: 0.3 LCMS (ESI): m/z 581.3 [M+H]⁺

### 3-Phenylpropyl ((2S)-1-(((2S)-4-(ethylamino)-3-hydroxy-4-oxo-1-((S)-2-oxopyrrolidin-3-yl) butan-2-yl) amino)-1-oxo-3-phenylpropan-2-yl) carbamate (9)

To a stirred solution of (6S,9S)-9-benzyl-4,8,11-trioxo-6-(((S)-2-oxopyrrolidin-3-yl) methyl)-15-phenyl-12-oxa-3,7,10-triazapentadecan-5-yl acetate (8) (290 mg, 0.499 mmol) in THF: H₂O (3 mL:1 mL), was added LiOH (18 mg, 0.749 mmol), at 0° C. and stirred at RT for 3 h. The progress of the reaction was monitored by TLC. Reaction mixture was concentrated and acidified with 1N HCl and extracted with EtOAc (2×20 mL). Organic layer was dried over anhydrous Na₂SO₄ and evaporated under reduced pressure to afford material: This material was purified by reverse phase chromatography by using 0.1% ABC in water: acetonitrile to afford pure 3-phenylpropyl ((2S)-1-(((2S)-4-(ethylamino)-3-hydroxy-4-oxo-1-((S)-2-oxopyrrolidin-3-yl) butan-2-yl) amino)-1-oxo-3-phenylpropan-2-yl) carbamate (9). TLC system: 10% MeOH in DCM Rf: 0.4 LCMS (ESI): m/z 539.6 [M+H]⁺

### 3-Phenylpropyl ((S)-1-(((S)-4-(ethylamino)-3,4-dioxo-1-((S)-2-oxopyrrolidin-3-yl) butan-2-yl) amino)-1-oxo-3-phenylpropan-2-yl) carbamate (A276)

To a stirred solution of 3-phenylpropyl ((2S)-1-(((2S)-4-(ethylamino)-3-hydroxy-4-oxo-1-((S)-2-oxopyrrolidin-3-yl) butan-2-yl) amino)-1-oxo-3-phenylpropan-2-yl) carbamate (9) (100 mg, 0.185 mmol) in ethyl acetate (5 mL) was added Dess-Martin periodinane (157 mg, 0.371 mmol) at 0° C. and stirred at RT for 3 h. The progress of the reaction was monitored by TLC and LCMS. Reaction mixture was filter through diatomaceous earth pad and washed with ethyl acetate (15 mL) and filtrate was washed with hypo solution (3×20 mL) followed by saturated NaHCO₃ solution (3×20 mL). Organic layer was dried over anhydrous Na₂SO₄, filtered and concentrated to get residue. The compound was purified by reverse phase chromatography by using 0.1% ABC in water: acetonitrile to afford 3-phenylpropyl ((S)-1-(((S)-4-(ethylamino)-3,4-dioxo-1-((S)-2-oxopyrrolidin-3-yl) butan-2-yl) amino)-1-oxo-3-phenylpropan-2-yl) carbamate (A276). TLC system: 10% MeOH in DCM Rf: 0.6 LCMS (ESI): m/z 537.3 [M+H]⁺

### Example 56: Synthesis of Compound A278

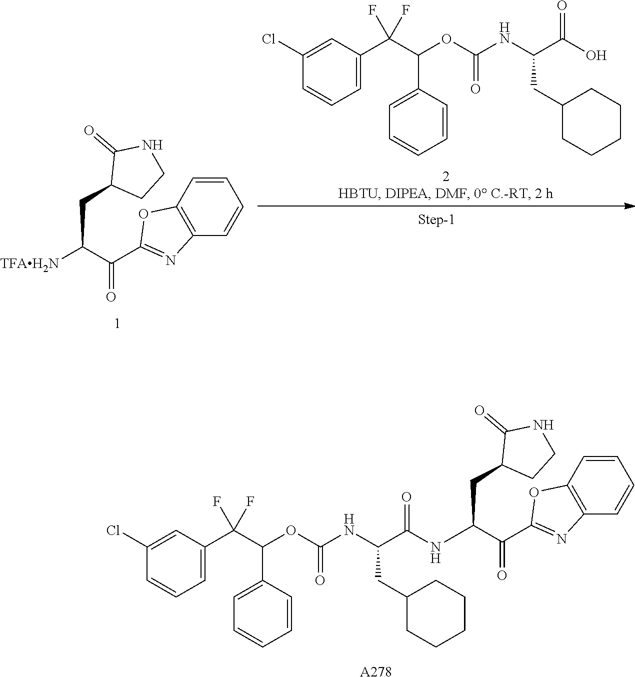

### 2-(3-chlorophenyl)-2,2-difluoro-1-phenylethyl ((S)-1-(((S)-1-(benzo[d]oxazol-2-yl)-1-oxo-3-((S)-2-oxopyrrolidin-3-yl)propan-2-yl)amino)-3-cyclohexyl-1-oxopropan-2-yl)carbamate (A278)

To a stirred solution of (S)-3-((S)-3-(benzo[d]oxazol-2-yl)-3-oxo-2-((2,2,2-trifluoroacetyl)-14-azaneyl)propyl)pyrrolidin-2-one as a (TFA salt) (1) (0.14 g, 0.37 mmol) in DMF (5 mL) at 0° C. was added (2S)-2-(((2-(3-chlorophenyl)-2,2-difluoro-1-phenylethoxy)carbonyl)amino)-3-cyclohexyl propanoic acid (0.176 g, 0.37 mmol), HBTU (0.143 g, 0.37 mmol, 1 eq), DIPEA (0.62 mL, 1.13 mmol) and resulting reaction mixture was stirred at RT for 2 h. After completion of the reaction (monitored by TLC and LC-MS), the reaction mixture was diluted with water (50 mL) and extracted with EtOAc (2×25 mL). The combined organic layer was washed with brine solution (50 mL), dried over anhy. Na₂SO₄ and concentrated under reduced pressure. Obtained material was purified by prep HPLC to afford 2-(3-chlorophenyl)-2,2-difluoro-1-phenylethyl ((S)-1-(((S)-1-(benzo[d]oxazol-2-yl)-1-oxo-3-((S)-2-oxopyrrolidin-3-yl)propan-2-yl)amino)-3-cyclo hexyl-1-oxopropan-2-yl)carbamate (A278). TLC system: 80% EA/Pet ether Rf: 0.2 LCMS (ESI): m/z 721.3 [M+H]⁺

### Example 57: Synthesis of Compound A279

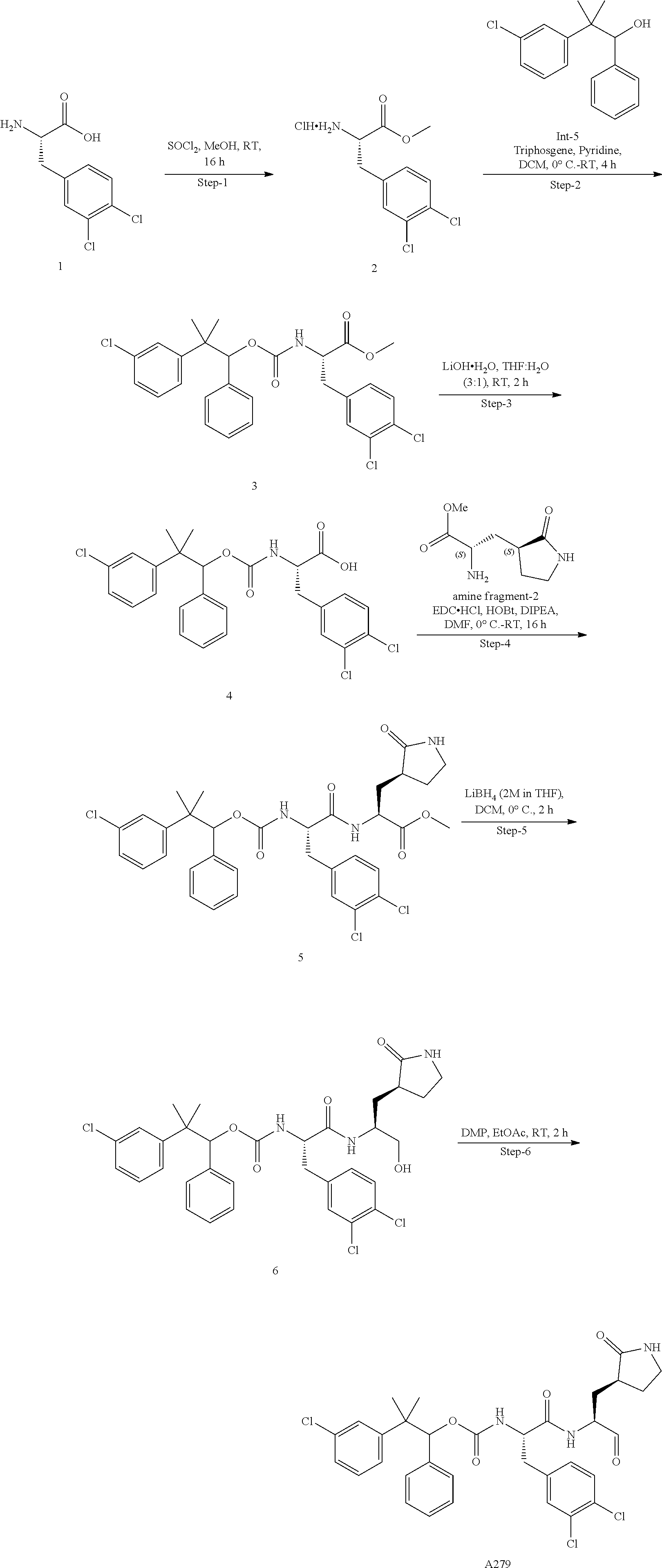

### methyl (S)-2-amino-3-(3,4-dichlorophenyl)propanoate hydrochloride (2)

To a stirred solution of (S)-2-amino-3-(3,4-dichlorophenyl)propanoic acid (1) (5.0 g, 21.45 mmol) in MeOH (50 mL) was added SOCl₂ (4.67 mL) at 0° C. and stirred at RT for 16 h. The progress of the reaction was monitored by TLC. After completion of starting material, reaction mixture was concentrated under reduced pressure to afford methyl (S)-2-amino-3-(3,4-dichlorophenyl) propanoate hydrochloride (2). TLC system: 10% MeOH/DCM Rf: 0.1 LCMS (ESI): m/z 247.98 [M+H]⁺

### methyl (2S)-2-(((2-(3-chlorophenyl)-2-methyl-1-phenylpropoxy)carbonyl)amino)-3-(3,4-dichlorophenyl)propanoate (3)

To a stirred solution of 2-(3-chlorophenyl)-2-methyl-1-phenylpropan-1-ol (Int-5) (3.0 g, 11.53 mmol) in DCM (30 mL) was added Pyridine (9 mL, 115.38 mmol) and Cpd-2 (3.9 g, 13.84 mmol) followed by Triphosgene (2.73 g, 9.22 mmol) added portion wise at 0° C. and stirred the reaction mixture at room temperature for 4 h. The progress of the reaction was monitored by TLC. Reaction mixture was quenched with 1N HCl (50 mL) and extracted with ethyl acetate (2×75 mL). Combined organic layer was washed with brine solution (50 mL), dried over anhy. Na2SO4 and evaporated under reduced pressure. The material was purified by silica gel (230-400 mesh) column chromatography to afford methyl (2S)-2-(((2-(3-chlorophenyl)-2-methyl-1-phenylpropoxy)carbonyl)amino)-3-(3,4-dichlorophenyl)propanoate (3). TLC system: 10% Ethyl acetate in hexane Rf: 0.55

### (2S)-2-(((2-(3-chlorophenyl)-2-methyl-1-phenylpropoxy)carbonyl)amino)-3-(3,4-dichlorophenyl)propanoic acid (4)

To a stirred solution of methyl (2S)-2-(((2-(3-chlorophenyl)-2-methyl-1-phenylpropoxy) carbonyl)amino)-3-(3,4-dichlorophenyl)propanoate (3) (2.9 g, 5.44 mmol) in THE (20 mL) and water (7 mL) was added LiOH·H₂O (0.34 g, 8.16 mmol) at RT and stirred at room temperature for 2 h. The progress of the reaction was monitored by TLC. THE was distilled under reduced pressure, compound was acidified with aq. 1N HCl solution up to pH˜2 and extracted with DCM (2×50 mL). Combined organic layer was washed with water (50 mL), brine solution (50 mL), dried over anhy. sodium sulfate and concentrated under reduced pressure to afford (2S)-2-(((2-(3-chlorophenyl)-2-methyl-1-phenylpropoxy)carbonyl)amino)-3-(3,4-dichlorophenyl) propa noic acid (4). TLC system: 10% Methanol in dichloromethane Rf: 0.1 LCMS (ESI): m/z 518.26 [M−H]⁺

### methyl (2S)-2-((2S)-2-(((2-(3-chlorophenyl)-2-methyl-1-phenylpropoxy)carbonyl)amino)-3-(3,4-dichlorophenyl)propanamido)-3-((S)-2-oxopyrrolidin-3-yl)propanoate (5)

To a solution of (2S)-2-(((2-(3-chlorophenyl)-2-methyl-1-phenylpropoxy) carbonyl) amino)-3-(3,4-dichlorophenyl) propanoic acid (4) (2.5 g, 4.8 mmol) in DMF (2.5 mL) was added EDC·HCl (1.38 g, 7.2 mmol), HOBt (0.972 g 7.2 mmol), DIPEA (2.5 mL, 14.4 mmol) and methyl (S)-2-amino-3-((S)-2-oxopyrrolidin-3-yl)propanoate hydrochloride (amine fragment-2) (1.28 g, 5.78 mmol) at 0° C. and stirred at room temperature for 4 h. Reaction mixture was diluted with ice water (50 mL) and extracted with ethyl acetate (2×50 mL). Combined organic layer was dried over anhydrous sodium sulphate and evaporated under reduced pressure. Obtained residue was purified by column chromatography using silica gel (230-400 mesh) to afford methyl (2S)-2-((2S)-2-(((2-(3-chlorophenyl)-2-methyl-1-phenylpropoxy)carbonyl)amino)-3-(3,4-dichlorophenyl) propan amido)-3-((S)-2-oxopyrrolidin-3-yl)propanoate (5). TLC system: 5% MeOH/DCM Rf: 0.45 LCMS (ESI): m/z=688.3 [M+H]⁺

### 2-(3-chlorophenyl)-2-methyl-1-phenylpropyl ((S)-3-(3,4-dichlorophenyl)-1-(((S)-1-hydroxy-3-((S)-2-oxopyrrolidin-3-yl)propan-2-yl)amino)-1-oxopropan-2-yl)carbamate (6)

To a stirred solution of methyl (2S)-2-((2S)-2-(((2-(3-chlorophenyl)-2,2-difluoro-1-phenylethoxy)carbonyl)amino)-3-(3,4-dichlorophenyl)propanamido)-3-((S)-2-oxopyrrolidin-3-yl)propanoate (5) (2.3 g, 3.34 mmol) in THE (23 mL) was added LiBH₄ (2M in THF, 3.34 mL, 6.69 mmol) slowly drop wise at 0° C. and stirred for 2 h at 0° C. The progress of the reaction was monitored by TLC. After completion of starting material, reaction mixture was quenched with saturated NH₄Cl solution and extracted with dichloromethane (2×150 mL). Combined organic layer was washed with water, dried over anhydrous sodium sulfate and concentrated under reduced pressure and purified by reverse phase column (C18) eluting with 60% ACN in 0.1% FA in water to afford 2-(3-chlorophenyl)-2-methyl-1-phenylpropyl ((S)-3-(3,4-dichlorophenyl)-1-(((S)-1-hydroxy-3-((S)-2-oxopyrrolidin-3-yl)propan-2-yl)amino)-1-oxo propan-2-yl)carbamate (6). TLC system: 5% Methanol in dichloromethane Rf: 0.35 LCMS (ESI): m/z 660.4 [M+H]⁺

### 2-(3-chlorophenyl)-2-methyl-1-phenylpropyl ((S)-3-(3,4-dichlorophenyl)-1-oxo-1-(((S)-1-oxo-3-((S)-2-oxopyrrolidin-3-yl)propan-2-yl)amino)propan-2-yl)carbamate (A279)

To a stirred solution of 2-(3-chlorophenyl)-2-methyl-1-phenylpropyl ((S)-3-(3,4-dichlorophenyl)-1-(((S)-1-hydroxy-3-((S)-2-oxopyrrolidin-3-yl)propan-2-yl)amino)-1-oxo propan-2-yl)carbamate (6) (400 mg, 0.606 mmol) in ethyl acetate (4 mL) was added Dess-Martin periodinane (386 mg, 0.91 mmol) slowly portion wise at 0° C. and stirred at RT for 2 h. The progress of the reaction was monitored by TLC and LCMS. Reaction mixture was diluted with Ethyl acetate (20 mL) and filtered through diatomaceous earth pad and filtrate was washed with sat. sodium thiosulfate solution (3×20 mL) followed by sat. NaHCO₃ solution (3×20 mL). Organic layer was dried over anhydrous Na₂SO₄, filtered and concentrated under reduced pressure. Obtained material was purified by reverse phase combiflash column (C18) using 60% ACN in 0.1% aq. NH₄CO₃ solution as eluent to afford 2-(3-chlorophenyl)-2-methyl-1-phenylpropyl ((S)-3-(3,4-dichlorophenyl)-1-oxo-1-(((S)-1-oxo-3-((S)-2-oxopyrrolidin-3-yl)propan-2-yl)amino)propan-2-yl)carbamate (A279). TLC system: 10% Methanol in DCM Rf: 0.55 LCMS (ESI): m/z 658.2 [M+H]⁺

### Example 58: Synthesis of Compound A280

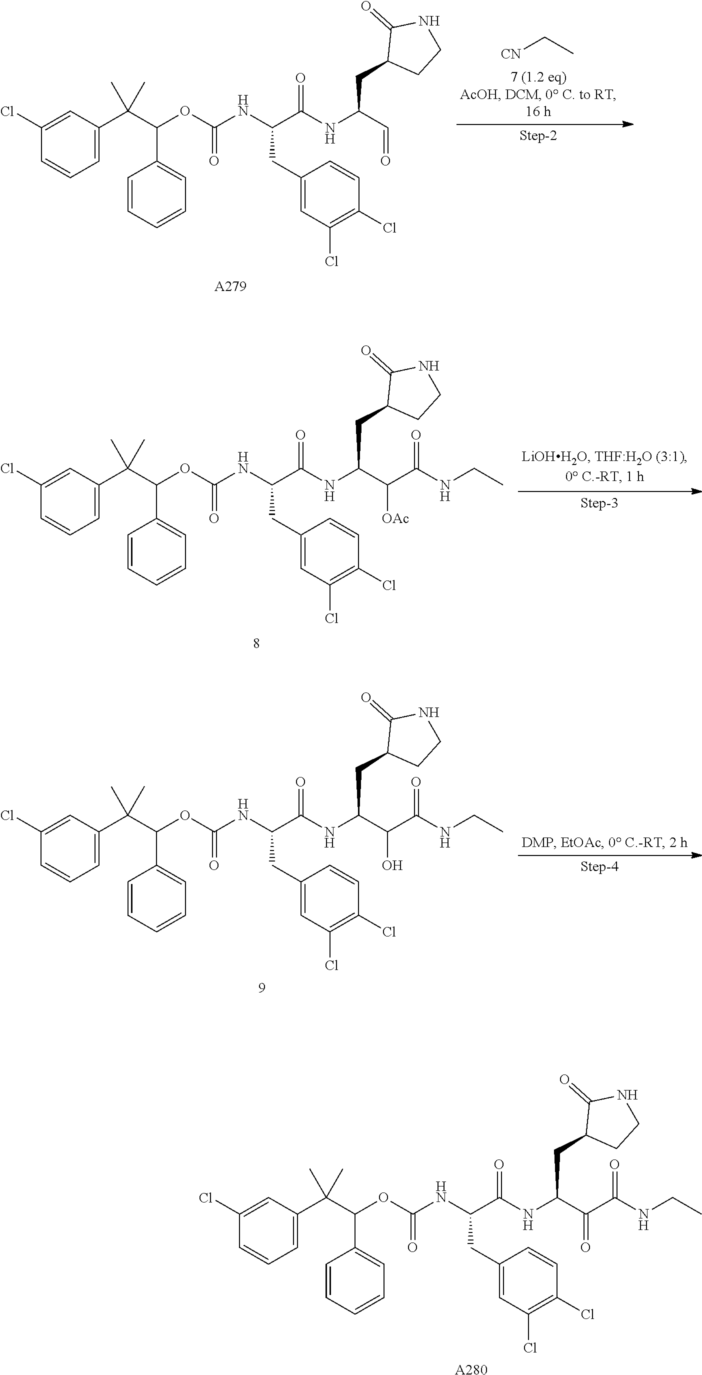

### (6S,9S)-14-(3-chlorophenyl)-9-(3,4-dichlorobenzyl)-14-methyl-4,8,11-trioxo-6-(((S)-2-oxopyrrolidin-3-yl)methyl)-13-phenyl-12-oxa-3,7,10-triazapentadecan-5-yl acetate (8)

To a stirred solution of 2-(3-chlorophenyl)-2-methyl-1-phenylpropyl ((S)-3-(3,4-dichlorophenyl)-1-oxo-1-(((S)-1-oxo-3-((S)-2-oxopyrrolidin-3-yl)propan-2-yl)amino) propan-2-yl)carbamate (A279) (0.6 g, 0.913 mmol) in DCM (6 mL) was added isocyanoethane (7) (150 mg, 2.73 mmol) followed by acetic acid (0.22 mL, 3.65 mmol) at 0° C. and stirred at RT for 6 h. The progress of the reaction was monitored by TLC and LCMS. After completion of starting material, reaction mixture was diluted with dichloromethane and washed with sat. ammonium chloride solution (2×20 mL) followed by brine (1×20 mL). Organic layer was dried over anhydrous Na₂SO₄ and evaporated under reduced pressure. Obtained material was purified by reverse phase combi flash column (C18) using 60% ACN in 0.1% FA in water as eluent to afford (6S,9S)-14-(3-chlorophenyl)-9-(3,4-dichlorobenzyl)-14-methyl-4,8,11-trioxo-6-(((S)-2-oxopyrrolidin-3-yl)methyl)-13-phenyl-12-oxa-3,7,10-triazapentadecan-5-yl acetate (8). TLC system: 10% MeOH in DCM Rf: 0.5 LCMS (ESI): m/z 773.42 [M+H]⁺

### 2-(3-chlorophenyl)-2-methyl-1-phenylpropyl ((2S)-3-(3,4-dichlorophenyl)-1-(((2S)-4-(ethyl amino)-3-hydroxy-4-oxo-1-((S)-2-oxopyrrolidin-3-yl)butan-2-yl)amino)-1-oxopropan-2-yl)carbamate (9)

To a stirred solution of (6S,9S)-14-(3-chlorophenyl)-9-(3,4-dichlorobenzyl)-14-methyl-4,8,11-trioxo-6-(((S)-2-oxopyrrolidin-3-yl)methyl)-13-phenyl-12-oxa-3,7,10-triazapentadecan-5-yl acetate (8) (400 mg, 0.518 mmol) in THE (3 mL) and water (1 mL) was added LiOH·H₂O (32.6 mg, 0.77 mmol) at 0° C. and stirred at room temperature for 1 h. The progress of the reaction was monitored by TLC and LCMS. After completion of the reaction, reaction mixture was extracted with ethyl acetate (2×50 mL). Combined organic layer was washed with brine solution (30 mL), dried over anhy. sodium sulfate and concentrated under reduced pressure to afford 2-(3-chlorophenyl)-2-methyl-1-phenylpropyl ((2S)-3-(3,4-dichlorophenyl)-1-(((2S)-4-(ethyl amino)-3-hydroxy-4-oxo-1-((S)-2-oxopyrrolidin-3-yl)butan-2-yl)amino)-1-oxopropan-2-yl)carbamate (9). TLC system: 10% MeOH in DCM Rf: 0.4 LCMS (ESI): m/z 731.6 [M+H]+

### 2-(3-chlorophenyl)-2-methyl-1-phenylpropyl ((S)-3-(3,4-dichlorophenyl)-1-(((S)-4-(ethylamino)-3,4-dioxo-1-((S)-2-oxopyrrolidin-3-yl)butan-2-yl)amino)-1-oxopropan-2-yl)carbamate (A280)

To a stirred solution of 2-(3-chlorophenyl)-2-methyl-1-phenylpropyl ((2S)-3-(3,4-dichlorophenyl)-1-(((2S)-4-(ethylamino)-3-hydroxy-4-oxo-1-((S)-2-oxopyrrolidin-3-yl) butan-2-yl)amino)-1-oxopropan-2-yl)carbamate (9) (230 mg, 0.315 mmol) in ethyl acetate (2.5 mL) was added Dess-Martin periodinane (200 mg, 0.47 mmol) at 0° C. and stirred at RT for 2 h. The progress of the reaction was monitored by TLC and LCMS. Reaction mixture was filtered through diatomaceous earth bed and washed with ethyl acetate (10 mL). Obtained filtrate was washed with sat. sodium thiosulfate solution (3×20 mL) followed by sat. NaHCO₃ solution (3×20 mL). Organic layer was dried over anhydrous Na₂SO₄, filtered and concentrated under reduced pressure. Obtained residue was purified by trituration with n-Penatne/DEE to afford 2-(3-chlorophenyl)-2-methyl-1-phenylpropyl ((S)-3-(3,4-dichlorophenyl)-1-(((S)-4-(ethylamino)-3,4-dioxo-1-((S)-2-oxopyrrolidin-3-yl)butan-2-yl)amino)-1-oxopropan-2-yl)carbamate (A280). TLC system: 10% MeOH in DCM Rf: 0.65 LCMS (ESI): m/z 729.2 [M+H]+

### Example 59: Synthesis of Compound A281

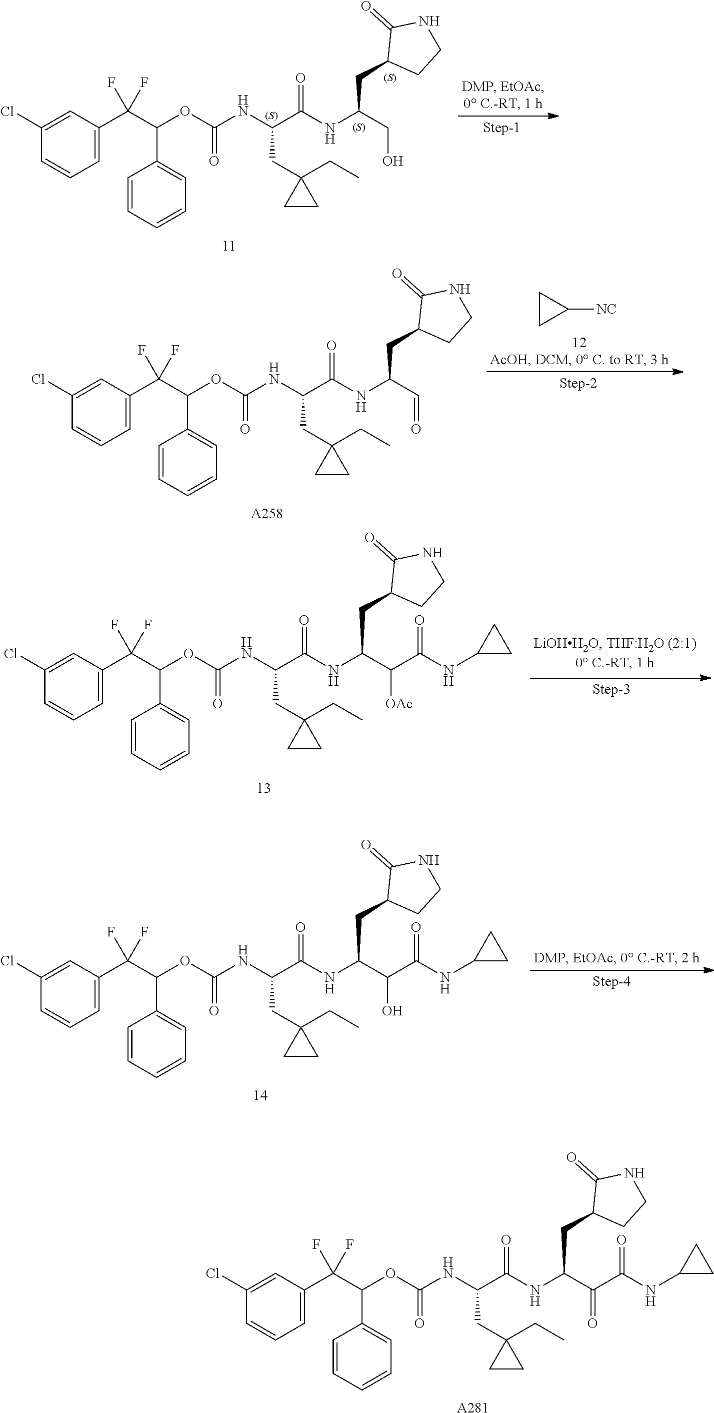

### 2-(3-chlorophenyl)-2,2-difluoro-1-phenylethyl ((S)-3-(1-ethylcyclopropyl)-1-oxo-1-(((S)-1-oxo-3-((S)-2-oxopyrrolidin-3-yl)propan-2-yl)amino)propan-2-yl)carbamate (A258)

To a stirred solution of 2-(3-chlorophenyl)-2,2-difluoro-1-phenylethyl ((S)-3-(1-ethylcyclopropyl)-1-(((S)-1-hydroxy-3-((S)-2-oxopyrrolidin-3-yl)propan-2-yl)amino)-1-oxopropan-2-yl)carbamate (11) (580 mg, 0.98 mmol) in ethyl acetate (5.8 mL) was added Dess-Martin period inane (830 mg, 1.96 mmol) slowly portion wise at 0° C. and stirred at RT for 1 h. The progress of the reaction was monitored by TLC and LCMS. Reaction mixture was diluted with Ethyl acetate (50 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure to afford 2-(3-chlorophenyl)-2,2-difluoro-1-phenylethyl ((S)-3-(1-ethylcyclopropyl)-1-oxo-1-(((S)-1-oxo-3-((S)-2-oxopyrrolidin-3-yl)propan-2-yl)amino)propan-2-yl)carbamate (A258). TLC system: 10% Methanol in DCM Rf: 0.55 LCMS (ESI): m/z 590.3 [M+H]⁺

### (3S)-3-((2S)-2-(((2-(3-chlorophenyl)-2,2-difluoro-1-phenylethoxy)carbonyl)amino)-3-(1-ethylcyclopropyl)propanamido)-1-(cyclopropylamino)-1-oxo-4-((S)-2-oxopyrrolidin-3-yl)butan-2-yl acetate (13)

To a stirred solution of 2-(3-chlorophenyl)-2,2-difluoro-1-phenylethyl ((S)-3-(1-ethylcyclopropyl)-1-oxo-1-(((S)-1-oxo-3-((S)-2-oxopyrrolidin-3-yl)propan-2-yl)amino)propan-2-yl)carbamate (A258) (500 mg, 0.84 mmol) in dichloromethane (5 mL) was added acetic acid (152.7 mg 2.54 mmol) and cyclopropyl isocyanide (12) (170.5 mg, 2.54 mmol) at 0° C. and stirred at RT for 3 h. Reaction was monitored by TLC and LCMS. After completion of the reaction, reaction mixture was diluted with DCM (20 mL) and washed with water (3×40 mL), brine solution. Organic layer was separated, dried over anhy. Na₂SO₄, filtered and concentrated under reduced pressure. Obtained material was purified by reverse phase comb flash column (C18) using 50% ACN in 0.1% aq. NH₄CO₃ solution as eluent to afford (3S)-3-((2S)-2-(((2-(3-chlorophenyl)-2,2-difluoro-1-phenylethoxy)carbonyl)amino)-3-(1-ethylcyclopropyl) propan amido)-1-(cyclopropylamino)-1-oxo-4-((S)-2-oxopyrrolidin-3-yl)butan-2-yl acetate (13). TLC system: 10% Methanol in dichloromethane Rf: 0.4 LCMS (ESI): m/z 717.63 (M+H)⁺

### 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-3-(1-ethylcyclopropyl)-1-oxopropan-2-yl)carbamate (14)

To a stirred solution of (3S)-3-((2S)-2-(((2-(3-chlorophenyl)-2,2-difluoro-1-phenylethoxy) carbonyl)amino)-3-(1-ethylcyclopropyl) propan amido)-1-(cyclopropylamino)-1-oxo-4-((S)-2-oxopyrrolidin-3-yl)butan-2-yl acetate (13) (330 mg, 0.46 mmol) in THE (2 mL) and water (1 mL) was added LiOH·H₂O (29 mg, 0.69 mmol) at 0° C. and stirred at room temperature for 1 h. The progress of the reaction was monitored by TLC and LCMS. After completion of the reaction, reaction mixture was extracted with ethyl acetate (2×100 mL) and washed with water (30 mL). Combined organic layer was dried over anhy. Na₂SO₄ and concentrated under reduced pressure to afford 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-3-(1-ethylcyclopropyl)-1-oxopropan-2-yl)carbamate (14). TLC system: 10% Methanol in dichloromethane Rf: 0.3 LCMS (ESI): m/z 675.58 (M+H)⁺

### 2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-3-(1-ethylcyclopropyl)-1-oxopropan-2-yl)carbamate (A281)

To a stirred solution of 2-(3-chlorophenyl)-2,2-difluoro-1-phenylethyl ((2S)-1-(((2S)-4-(cyclopropylamino)-3-hydroxy-4-oxo-1-((S)-2-oxopyrrolidin-3-yl)butan-2-yl)amino)-3-(1-ethylcyclopropyl)-1-oxopropan-2-yl)carbamate (14) (300 mg, 0.44 mmol) in Ethyl acetate (3 mL) was added Dess-Martin periodinane (377 mg, 0.88 mmol) at 0° C. and stirred at RT for 2 h. After completion of the reaction, reaction mixture was diluted with Ethyl acetate (20 mL) and filtered through calcite pad. Obtained filtrate was washed with sat. Hypo solution (3×50 mL) followed by sat. NaHCO₃ solution (3×50 mL). Organic layer was separated, dried over anhydrous Na₂SO₄, filtered and concentrated under reduced pressure. Obtained compound was purified by trituration with n-Pentane/DEE to afford 2-(3-chlorophenyl)-2,2-difluoro-1-phenylethyl ((S)-1-(((S)-4-(cyclopropylamino)-3,4-dioxo-1-((S)-2-oxopyrrolidin-3-yl)butan-2-yl)amino)-3-(1-ethylcyclopropyl)-1-oxopropan-2-yl)carbamate (A281). TLC system: 5% Methanol in DCM Rf: 0.5 LCMS (ESI): m/z 673.3 [M+H]⁺

### Example 60: Synthesis of Intermediate I225

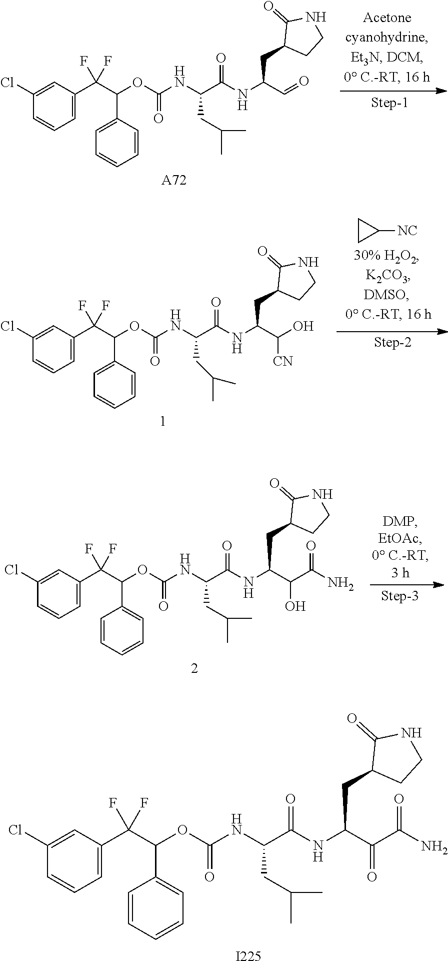

### 2-(3-chlorophenyl)-2,2-difluoro-1-phenylethyl ((2S)-1-(((2S)-1-cyano-1-hydroxy-3-((S)-2-oxopyrrolidin-3-yl)propan-2-yl)amino)-4-methyl-1-oxopentan-2-yl)carbamate

To a stirred solution of 2-(3-chlorophenyl)-2,2-difluoro-1-phenylethyl ((S)-4-methyl-1-oxo-1-(((S)-1-oxo-3-((S)-2-oxopyrrolidin-3-yl) propan-2-yl) amino) pentan-2-yl)carbamate (A72) (420 mg 7.44 mmol) in DCM (10 mL) was added acetonecyanohydrine (0.42 mL, 1 vol), Et₃N (0.42 mL, 1 vol) at 0° C. and stirred at RT for 16 h. The progress of the reaction was monitored by TLC. The reaction mixture was diluted with DCM (15 mL) and washed with water (2×15 mL), dried over sodium sulfate and evaporated under reduced pressure to get the product. This was triturated with n-pentane (15 mL) to afford 2-(3-chlorophenyl)-2,2-difluoro-1-phenylethyl ((2S)-1-(((2S)-1-cyano-1-hydroxy-3-((S)-2-oxopyrrolidin-3-yl) propan-2-yl) amino)-4-methyl-1-oxopentan-2-yl) carbamate. TLC system: 10% Methanol in dichloromethane Rf: 0.6 LCMS (ESI): m/z 591.35 [M+H]⁺

### 2-(3-chlorophenyl)-2,2-difluoro-1-phenylethyl ((2S)-1-(((2S)-4-amino-3-hydroxy-4-oxo-1-((S)-2-oxopyrrolidin-3-yl)butan-2-yl)amino)-4-methyl-1-oxopentan-2-yl)carbamate

To a stirred solution of 2-(3-chlorophenyl)-2,2-difluoro-1-phenylethyl ((2S)-1-(((2S)-1-cyano-1-hydroxy-3-((S)-2-oxopyrrolidin-3-yl) propan-2-yl) amino)-4-methyl-1-oxopentan-2-yl) carbamate (1) 300 mg, 5.07 mmol) in DMSO (5 mL) was added potassium carbonate (90 mg, 7.614 mmol) followed by 30% H₂O₂ (0.8 mL, 2 vol) at 0° C. and stirred at RT for 16 h. The progress of the reaction was monitored by TLC. The reaction mixture was quenched with Sat. ammonium chloride solution (20 mL) and extracted with ethyl acetate (2×20 mL). The combined organic layer was washed with water (2×15 mL) followed by brine (1×15 mL), dried over sodium sulfate and evaporated under reduced pressure to get the material, which was triturated with n-pentane (15 mL) to afford 2-(3-chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-4-methyl-1-oxopentan-2-yl) carbamate. TLC system: 10% Methanol in dichloromethane Rf: 0.3 LCMS (ESI): m/z 609.39 [M+H]⁺

### 2-(3-chlorophenyl)-2,2-difluoro-1-phenylethyl ((S)-1-(((S)-4-amino-3,4-dioxo-1-((S)-2-oxopyrrolidin-3-yl) butan-2-yl) amino)-4-methyl-1-oxopentan-2-yl) carbamate

To a stirred solution of 2-(3-chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-4-methyl-1-oxopentan-2-yl) carbamate (2) (250 mg, 4.105 mmol) in ethyl acetate (10 mL) was added Dess-Martin periodinane (522 mg, 1.231 mmol) at 0° C. and stirred at RT for 3 h The progress of the reaction was monitored by TLC and LC-MS. Reaction mixture was filtered through diatomaceous earth bed and washed with ethyl acetate (20 mL) Filtrate was washed with with sat Hypo solution (3×20 mL), sat NaHCO₃ solution (3×20 mL) followed by brine (1×15 mL). Organic layer was dried over anhydrous Na₂SO₄, filtered and concentrated to get material, which was purified by reverse phase chromatography by using 0.1% ABC in acetonitrile as buffer to afford 2-(3-chlorophenyl)-2,2-difluoro-1-phenylethyl ((S)-1-(((S)-4-amino-3,4-dioxo-1-((S)-2-oxopyrrolidin-3-yl) butan-2-yl) amino)-4-methyl-1-oxopentan-2-yl) carbamate (I225). TLC system: 10% Methanol in dichloromethane Rf: 0.4 LCMS (ESI): m/z 607.3 [M+H]⁺

### Example 61: Synthesis of Compounds A282 and A273

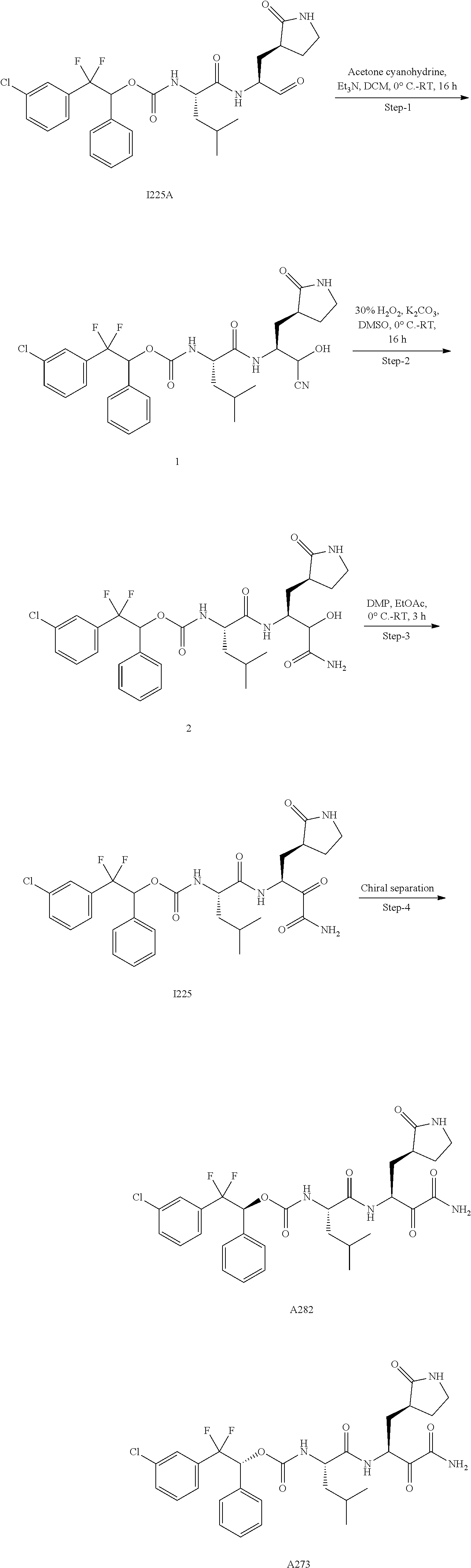

### 2-(3-Chlorophenyl)-2,2-difluoro-1-phenylethyl ((2S)-1-(((2S)-1-cyano-1-hydroxy-3-((S)-2-oxopyrrolidin-3-yl)propan-2-yl)amino)-4-methyl-1-oxopentan-2-yl)carbamate

To a stirred solution of 2-(3-chlorophenyl)-2,2-difluoro-1-phenylethyl ((S)-4-methyl-1-oxo-1-(((S)-1-oxo-3-((S)-2-oxopyrrolidin-3-yl)propan-2-yl) amino) pentan-2-yl)carbamate (I225A) (1.0 g, 1.7761 mmol) in DCM (10 mL) was added acetonecyanohydrine (1 mL, 1 vol), Et₃N (1 mL, 1 vol) at 0° C. and stirred at RT for 16 h. The progress of the reaction was monitored by TLC. The reaction mixture was diluted with DCM (15 mL) and washed with water (2×15 mL), dried over sodium sulfate and evaporated under reduced pressure to get the product. This material was triturated with n-pentane (15 mL) to afford 2-(3-chlorophenyl)-2,2-difluoro-1-phenylethyl ((2S)-1-(((2S)-1-cyano-1-hydroxy-3-((S)-2-oxopyrrolidin-3-yl) propan-2-yl) amino)-4-methyl-1-oxopentan-2-yl) carbamate. TLC system: 5% Methanol in dichloromethane Rf: 0.5 LCMS (ESI): m/z 591.30 [M+H]⁺

2-(3-chlorophenyl)-2,2-difluoro-1-phenylethyl ((2S)-1-(((2S)-4-amino-3-hydroxy-4-oxo-1-((S)-2-oxopyrrolidin-3-yl)butan-2-yl)amino)-4-methyl-1-oxopentan-2-yl)carbamate (2) To a stirred solution of 2-(3-chlorophenyl)-2,2-difluoro-1-phenylethyl ((2S)-1-(((2S)-1-cyano-1-hydroxy-3-((S)-2-oxopyrrolidin-3-yl) propan-2-yl) amino)-4-methyl-1-oxopentan-2-yl) carbamate (1) 1.0 g, 1.6949 mmol) in DMSO (5 mL) was added potassium carbonate (350 mg, 2.54 mmol) followed by 30% H₂O₂ (4 mL, 4 vol) at 0° C. and stirred at RT for 16 h. The progress of the reaction was monitored by TLC. The reaction mixture was quenched with water (25 mL) and extracted with ethyl acetate (2×75 mL). The combined organic layer was washed with water (2×15 mL) followed by brine (1×15 mL), dried over sodium sulfate and evaporated under reduced pressure to get the material. This material was triturated with n-pentane (15 mL) to afford 2-(3-chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-4-methyl-1-oxopentan-2-yl) carbamate. TLC system: 10% Methanol in dichloromethane Rf: 0.3 LCMS (ESI): m/z 609.30 [M+H]⁺

### 2-(3-chlorophenyl)-2,2-difluoro-1-phenylethyl ((S)-1-(((S)-4-amino-3,4-dioxo-1-((S)-2-oxopyrrolidin-3-yl) butan-2-yl) amino)-4-methyl-1-oxopentan-2-yl) carbamate

To a stirred solution of 2-(3-chlorophenyl)-2,2-difluoro-1-phenylethyl ((2S)-1-(((2S)-4-amino-3-hydroxy-4-oxo-1-((S)-2-oxopyrrolidin-3-yl) butan-2-yl) amino)-4-methyl-1-oxopentan-2-yl) carbamate (2) (700 mg, 1.1513 mmol) in ethyl acetate (14 mL) was added Dess-Martin periodinane (1.46 g, 3.45 mmol) at 0° C. and stirred at RT for 3 h The progress of the reaction was monitored by TLC. The suspension was filtered through diatomaceous earth bed and washed with ethyl acetate (20 mL) Filtrate was washed with sat Hypo solution (3×20 mL), sat NaHCO₃ solution (3×20 mL) followed by brine (1×15 mL). Organic layer was dried over anhydrous Na₂SO₄, filtered and concentrated to get material, which was purified by reverse phase chromatography by using 0.1% ABC in acetonitrile as buffer to afford 2-(3-chlorophenyl)-2,2-difluoro-1-phenylethyl ((S)-1-(((S)-4-amino-3,4-dioxo-1-((S)-2-oxopyrrolidin-3-yl) butan-2-yl) amino)-4-methyl-1-oxopentan-2-yl) carbamate (I225). TLC system: 10% Methanol in dichloromethane Rf: 0.4 LCMS (ESI): m/z 607.2 [M+H]⁺

### (S)-2-(3-chlorophenyl)-2,2-difluoro-1-phenylethyl ((S)-1-(((S)-4-amino-3,4-dioxo-1-((S)-2-oxopyrrolidin-3-yl)butan-2-yl)amino)-4-methyl-1-oxopentan-2-yl)carbamate (A282) & (R)-2-(3-chlorophenyl)-2,2-difluoro-1-phenylethyl ((S)-1-(((S)-4-amino-3,4-dioxo-1-((S)-2-oxopyrrolidin-3-yl)butan-2-yl)amino)-4-methyl-1-oxopentan-2-yl)carbamate (A273)

Intermediate I225 was purified by Chiral SFC to afford (S)-2-(3-chlorophenyl)-2,2-difluoro-1-phenylethyl ((S)-1-(((S)-4-amino-3,4-dioxo-1-((S)-2-oxopyrrolidin-3-yl)butan-2-yl)amino)-4-methyl-1-oxopentan-2-yl)carbamate (A282) (150 mg, 0.2475 mmol) and (R)-2-(3-chlorophenyl)-2,2-difluoro-1-phenylethyl ((S)-1-(((S)-4-amino-3,4-dioxo-1-((S)-2-oxopyrrolidin-3-yl)butan-2-yl)amino)-4-methyl-1-oxopentan-2-yl)carbamate (A273). TLC system: 10% Methanol in DCM Rf: 0.3 LCMS (ESI): m/z 607.3 [M+H]⁺

### Example 62: Synthesis of Compounds A283 and A284

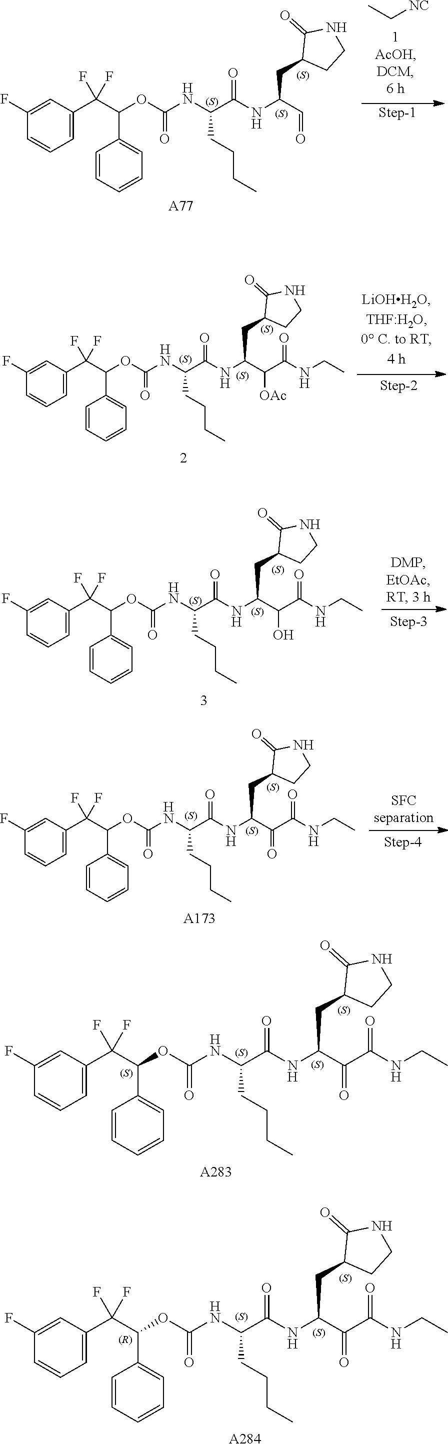

### (6S,9S)-6-butyl-1,1-difluoro-1-(3-fluorophenyl)-4,7,11-trioxo-9-(((S)-2-oxopyrrolidin-3-yl)methyl)-2-phenyl-3-oxa-5,8,12-triazatetradecan-10-yl acetate (2)

To a stirred solution of 2,2-difluoro-2-(3-fluorophenyl)-1-phenylethyl ((S)-1-oxo-1-(((S)-1-oxo-3-((S)-2-oxopyrrolidin-3-yl)propan-2-yl)amino)hexan-2-yl)carbamate (A77) (3.7 g, 6.76 mmol) in DCM (37 mL) was added isocyanoethane (1) (1.1 g, 20.28 mmol) in DCM (17 mL) followed by acetic acid (1.62 mL, 27.05 mmol) at 0° C. and stirred at RT for 6 h. The progress of the reaction was monitored by TLC and LCMS. Reaction mixture was diluted with dichloromethane and washed with 1N HCl (2×40 mL) followed by brine (1×20 mL). Organic layer was dried over anhydrous Na₂SO₄ and evaporated under reduced pressure. Obtained material was purified by Reverse phase column chromatography and eluted with 40% of ACN in 0.1% Aq·Ammonium bicarbonate solution to afforded (6S,9S)-6-butyl-1,1-difluoro-1-(3-fluorophenyl)-4,7,11-trioxo-9-(((S)-2-oxopyrrolidin-3-yl)methyl)-2-phenyl-3-oxa-5,8,12-triazatetradecan-10-yl acetate (2). TLC system: 5% Methanol in dichloromethane Rf: 0.2 LCMS (ESI): m/z 663.41 [M+H]⁺

### 2,2-difluoro-2-(3-fluorophenyl)-1-phenylethyl ((2S)-1-(((2S)-4-(ethylamino)-3-hydroxy-4-oxo-1-((S)-2-oxopyrrolidin-3-yl)butan-2-yl)amino)-1-oxohexan-2-yl)carbamate (3)

To the stirred solution of (6S,9S)-6-butyl-1,1-difluoro-1-(3-fluorophenyl)-4,7,11-trioxo-9-(((S)-2-oxopyrrolidin-3-yl)methyl)-2-phenyl-3-oxa-5,8,12-triazatetradecan-10-yl acetate (2) (2 g, 3.02 mmol) in THE (20 mL), water (2 mL) was added LiOH·H₂O (190 mg, 4.5 mmol) at 0° C. and stirred at room temperature for 4 h. The progress of the reaction was monitored by TLC and LCMS. After completion of the reaction, reaction mixture was extracted with ethylacetate (2×100 mL), dried over anhy. sodium sulfate and concentrated under reduced pressure to afford 2,2-difluoro-2-(3-fluorophenyl)-1-phenylethyl ((2S)-1-(((2S)-4-(ethylamino)-3-hydroxy-4-oxo-1-((S)-2-oxo pyrrolidin-3-yl)butan-2-yl)amino)-1-oxohexan-2-yl)carbamate (3). TLC system: 5% Methanol in dichloromethane Rf: 0.1 LCMS (ESI): m/z 621.64 [M+H]⁺

### 2,2-difluoro-2-(3-fluorophenyl)-1-phenylethyl ((S)-1-(((S)-4-(ethylamino)-3,4-dioxo-1-((S)-2-oxopyrrolidin-3-yl)butan-2-yl)amino)-1-oxohexan-2-yl)carbamate (A173)

To a stirred solution of 2,2-difluoro-2-(3-fluorophenyl)-1-phenylethyl ((2S)-1-(((2S)-4-(ethylamino)-3-hydroxy-4-oxo-1-((S)-2-oxopyrrolidin-3-yl)butan-2-yl)amino)-1-oxohexan-2-yl)carbamate (3) (1 g, 1.61 mmol) in ethyl acetate (10 mL) was added Dess-Martin periodinane (1.36 g, 3.22 mmol) at 0° C. and stirred at RT for 3 h. The progress of the reaction was monitored by TLC and LCMS. After completion of the reaction, reaction mixture was filter through diatomaceous earth pad and washed the bed with ethyl acetate (30 mL). Obtained filtrate was washed with hypo solution (3×30 mL) followed by saturated aq. NaHCO₃ solution (3×20 mL). Organic layer was dried over anhydrous Na₂SO₄, filtered and concentrated to get material which was purified by reverse phase purification and 50% ACN in 0.1% aqueous formic acid to afford 2,2-difluoro-2-(3-fluorophenyl)-1-phenylethyl ((S)-1-(((S)-4-(ethylamino)-3,4-dioxo-1-((S)-2-oxopyrrolidin-3-yl)butan-2-yl)amino)-1-oxohexan-2-yl)carbamate (A173). TLC system: 10% Methanol in dichloromethane Rf: 0.4 LCMS (ESI): m/z 619.60 [M+H]⁺

### (S)-2,2-difluoro-2-(3-fluorophenyl)-1-phenylethyl ((S)-1-(((S)-4-(ethylamino)-3,4-dioxo-1-((S)-2-oxopyrrolidin-3-yl)butan-2-yl)amino)-1-oxohexan-2-yl)carbamate (A283) and (R)-2,2-difluoro-2-(3-fluorophenyl)-1-phenylethyl ((S)-1-(((S)-4-(ethylamino)-3,4-dioxo-1-((S)-2-oxopyrrolidin-3-yl)butan-2-yl)amino)-1-oxohexan-2-yl)carbamate (A284)

600 mg of 2,2-difluoro-2-(3-fluorophenyl)-1-phenylethyl ((S)-1-(((S)-4-(ethylamino)-3,4-dioxo-1-((S)-2-oxopyrrolidin-3-yl)butan-2-yl)amino)-1-oxohexan-2-yl)carbamate (A173) was purified by chiral SFC afforded (S)-2,2-difluoro-2-(3-fluorophenyl)-1-phenylethyl ((S)-1-(((S)-4-(ethylamino)-3,4-dioxo-1-((S)-2-oxopyrrolidin-3-yl)butan-2-yl)amino)-1-oxohexan-2-yl)carbamate (A283) and (R)-2,2-difluoro-2-(3-fluorophenyl)-1-phenylethyl ((S)-1-(((S)-4-(ethylamino)-3,4-dioxo-1-((S)-2-oxopyrrolidin-3-yl)butan-2-yl)amino)-1-oxohexan-2-yl)carbamate (A284). LCMS (ESI): m/z 619.3 [M+H]⁺

### Example 63: Synthesis of Compound A285

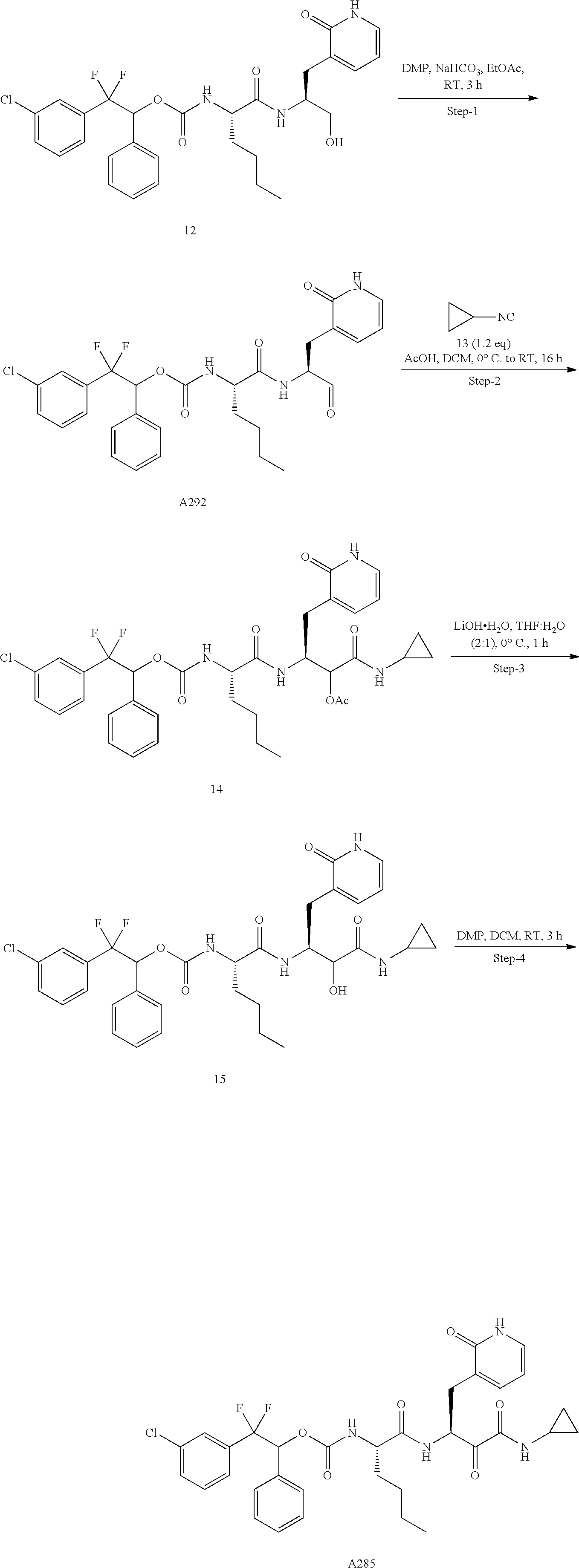

### 2-(3-chlorophenyl)-2,2-difluoro-1-phenylethyl ((S)-1-oxo-1-(((S)-1-oxo-3-(2-oxo-1,2-dihydro pyridin-3-yl)propan-2-yl)amino)hexan-2-yl)carbamate (A292)

To a stirred solution of 2(3-chlorophenyl)-2,2-difluoro-1-phenylethyl ((S)-1-(((S)-1-hydroxy-3-(2-oxo-1,2-dihydropyridin-3-yl)propan-2-yl)amino)-1-oxohexan-2-yl)carbamate (12) (500 mg, 0.86 mmol) in DCM (5 mL) was added NaHCO₃ (146 mg, 1.739 mmol) and Dess-Martin periodinane (737 mg, 1.739 mmol) slowly portion wise at 0° C. and stirred at RT for 3 h. The progress of the reaction was monitored by TLC and LCMS. Reaction mixture was diluted with Ethyl acetate (50 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure. Obtained material was triturated with DEE/Pentane (50%) to afford 2-(3-chlorophenyl)-2,2-difluoro-1-phenylethyl ((S)-1-oxo-1-(((S)-1-oxo-3-(2-oxo-1,2-dihydropyridin-3-yl)propan-2-yl)amino)hexan-2-yl)carbamate (A292). TLC system: 10% Methanol in DCM R_{f}: 0.55 LCMS (ESI): m/z 574.2 [M+H]⁺

### (3S)-3-((2S)-2-(((2-(3-chlorophenyl)-2,2-difluoro-1-phenylethoxy) carbonyl) amino) hexan amido)-1-(cyclopropylamino)-1-oxo-4-(2-oxo-1,2-dihydropyridin-3-yl)butan-2-yl acetate (14)

To a stirred solution of 2-(3-chlorophenyl)-2,2-difluoro-1-phenylethyl ((S)-1-oxo-1-(((S)-1-oxo-3-(2-oxo-1,2-dihydropyridin-3-yl)propan-2-yl)amino)hexan-2-yl)carbamate (A292) (500 mg, 2.61 mmol) in DCM (5 mL) was added isocyanocyclopropane (13) (0.5 g, 7.85 mmol) followed by Acetic acid (0.47 mL, 7.85 mmol) at 0° C. and stirred at RT for 16 h. The progress of the reaction was monitored by TLC. After 16 h, the reaction mixture was diluted with dichloromethane and washed with brine (15 mL), dried over sodium sulfate, concentrated under reduce pressure. This residue was purified by reverse phase combiflash column (C18) using 50% ACN in 0.1% aq. NH₄CO₃ solution as eluent to afford (3S)-3-((2S)-2-(((2-(3-chlorophenyl)-2,2-difluoro-1-phenylethoxy)carbonyl)amino)hexanamido)-1-(cyclopropylamino)-1-oxo-4-(2-oxo-1,2-dihydropyridin-3-yl)butan-2-yl acetate (14). TLC system: 10% Methanol in DCM R_{f}: 0.55 LCMS (ESI): m/z 701.66 [M+H]⁺

### 2-(3-chlorophenyl)-2,2-difluoro-1-phenylethyl ((2S)-1-(((2S)-4-(cyclopropylamino)-3-hydroxy-4-oxo-1-(2-oxo-1,2-dihydropyridin-3-yl) butan-2-yl)amino)-1-oxohexan-2-yl)carbamate (15)

To a stirred solution of (3S)-3-((2S)-2-(((2-(3-chlorophenyl)-2,2-difluoro-1-phenylethoxy) carbonyl)amino)hexanamido)-1-(cyclopropylamino)-1-oxo-4-(2-oxo-1,2-dihydropyridin-3-yl) butan-2-yl acetate (14) (300 mg, 0.42 mmol) in THE (3.0 mL), water (1.5 mL) was added LiOH·H₂O (26.9 mg, 0.64 mmol) at 0° C. and stirred at same temperature for 1 h. The progress of the reaction was monitored by TLC and LCMS. After completion of the reaction, reaction mixture was extracted with ethyl acetate (2×50 mL), combined organic layers were washed with brine solution (50 mL), dried over sodium sulfate, concentrated under reduced to afford 2-(3-chlorophenyl)-2,2-difluoro-1-phenylethyl ((2S)-1-(((2S)-4-(cyclopropylamino)-3-hydroxy-4-oxo-1-(2-oxo-1,2-dihydropyridin-3-yl)butan-2-yl)amino)-1-oxohexan-2-yl)carbamate (15). TLC system: 10% Methanol in DCM R_{f}: 0.55 LCMS (ESI): m/z 659.61 [M+H]⁺

### 2-(3-chlorophenyl)-2,2-difluoro-1-phenylethyl ((S)-1-(((S)-4-(cyclopropylamino)-3,4-dioxo-1-(2-oxo-1,2-dihydropyridin-3-yl)butan-2-yl)amino)-1-oxohexan-2-yl)carbamate (A285)

To a stirred solution of 2-(3-chlorophenyl)-2,2-difluoro-1-phenylethyl ((2S)-1-(((2S)-4-(cyclopropylamino)-3-hydroxy-4-oxo-1-(2-oxo-1,2-dihydropyridin-3-yl)butan-2-yl)amino)-1-oxohexan-2-yl)carbamate (15) (250 mg, 0.379 mmol) in DCM (2.5 mL) was added Dess-Martin periodinane (322 mg, 0.75 mmol) at 0° C. and stirred at RT for 3 h. The progress of the reaction was monitored by TLC. Reaction mixture was diluted with DCM (50 mL) and filtered through diatomaceous earth pad and filtrate was washed with sat. sodium thiosulfate solution (3×30 mL) followed by sat. NaHCO₃ solution (3×30 mL). Organic layer was dried over anhydrous Na₂SO₄, filtered and concentrated under reduced pressure. Obtained material was purified by reverse phase combiflash column (C18) using 50% ACN in 0.1% aq. NH₄CO₃ solution as eluent to afford to afford 2-(3-chlorophenyl)-2,2-difluoro-1-phenylethyl ((S)-1-(((S)-4-(cyclopropylamino)-3,4-dioxo-1-(2-oxo-1,2-dihydropyridin-3-yl)butan-2-yl)amino)-1-oxohexan-2-yl)carbamate (A285). TLC system: 10% Methanol in DCM R_{f}: 0.55 LCMS (ESI): m/z 657.3 [M+H]

### Testing of Activity of Compounds

### In Vitro Antiviral Assays

### Coronavirus Antiviral Assays:

Cell-based antiviral assays: The antiviral effects of inhibitors are examined in the Norwalk virus replicon harboring cells (HG23 cells). Briefly, confluent and semi-confluent cells are incubated with medium containing DMSO (<0.1%) or each compound (up to 100 μM) for 48 h. After the incubation, total RNA was extracted and viral genome is quantitated with real-time quantitative RT-PCR (qRT-PCR). The EC₅₀ values are determined by GraphPadPrism software. In addition to Norwalk virus replicon, the CPE (cytopathic effect) antiviral activities of the inhibitors are determined using FCoV (feline coronavirus), MERS-CoV (Middle East respiratory syndrome-related coronavirus), SARS-CoV (severe acute respiratory syndrome-related coronavirus), human coronavirus 229E, murine norovirus, and human rhinovirus.

Viral protease assays: The antiviral activities of inhibitors were determined by FRET (Fluorescence Resonance Energy Transfer) assay. Purified viral protease was incubated with the protease substrate peptide (Edans-DFHLQ/GP-Dabcyl) and inhibitor, and IC₅₀ values were subsequently determined by the fluorescence signals.

The SARS2 FRET protease assay was performed in 20 mM HEPES-Na pH 7, 120 mM NaCl, 0.4 mM EDTA, 0.01% Triton, 5% glycerol, and 4 mM DTT. A self-quenching peptide substrate 5-FAM-TSA VLQ SGF RKK (STAMRA)-NH2 was custom synthesized by Anaspec. Compounds were diluted in 4-fold serial dilutions to final concentrations of 20 μM to 0.075 nM. SARS2 protease was added to a final concentration of 30 nM, depending on the enzyme activity level, and the peptide substrate was added to a final concentration of 1.3 μM. The assay was incubated 120 minutes at 37° C. and read in a Perkin Elmer Envision with excitation at 492 nm and emission measurement at 518 nm.

Results of a SARS-CoV-2 protease inhibition assay are presented in Table C below.

TABLE C

| Compound | |
|---|---|
| no. | IC₅₀ (μM) |
| A1 | 0.0001 |
| A2 | 0.004 |
| A3 | 0.52 |
| A4 | 0.80 |
| A5 | 2.79 |
| A6 | 0.24 |
| A7 | 0.12 |
| A8 | 15.03 |
| A9 | 0.0001 |
| A10 | 0.005 |
| A11 | 0.0001 |
| A12 | 0.876 |
| A13 | 0.08 |
| A14 | 0.09 |
| A15 | 0.31 |
| A16 | 0.38 |
| A17 | 30.29 |
| A18 | 0.01 |
| A19 | 0.007 |
| A20 | 0.0003 |
| A20-2 | 0.0000002 |
| A21 | 0.06 |
| A22 | 0.32 |
| A24 | 21.41 |
| A25-1 | 0.000001 |
| A30 | 0.10 |
| A31 | 0.007 |
| A32 | 10.10 |
| A33 | 0.30 |
| A34 | 0.0001 |
| A35 | 1.07 |
| A36 | 1.15 |
| A37 | 0.91 |
| A38 | 0.0034 |
| A39 | 2.30 |
| A40 | 0.003 |
| A41 | 2.29 |
| A42 | 0.009 |
| A43 | 0.26 |
| A44 | 0.74 |
| A45 | 0.02 |
| A46 | 1.74 |
| A47 | 0.62 |
| A48 | 0.00003 |
| A49 | 0.38 |
| A50 | 0.01 |
| A51 | 0.13 |
| A52 | 1.42 |
| A53 | 0.00 |
| A54 | 0.14 |
| A55 | 0.01 |
| A56 | 0.56 |
| A57 | 1.13 |
| A58 | 0.16 |
| A59 | 16.15 |
| A60 | 1.91 |
| A61 | <0.0001 |
| A62 | 0.1479 |
| A63 | 0.0001 |
| A64 | 0.003 |
| A65 | 0.571 |
| A66 | 0.259 |
| A67 | 0.002 |
| A68 | 0.029 |
| A69 | 0.008 |
| A70 | 0.028 |
| A71 | 0.222 |
| A72 | <0.00001 |
| A73 | 1.395 |
| A74 | 0.186 |
| A75 | 0.009 |
| A76 | 0.053 |
| A77 | 0.009 |
| A78 | 0.009 |
| A79 | 0.013 |
| A80 | 0.14 |
| A81 | 0.47 |
| A83 | 0.51 |
| A84 | 0.02 |
| A85 | 0.46 |
| A86 | 0.37 |
| A87 | 0.09 |
| A88 | 0.63 |
| A89 | 0.02 |
| A90 | 0.04 |
| A91 | 0.01 |
| A92 | 1.12 |
| A93 | 0.0003 |
| A94 | 0.0123 |
| A95 | 0.0009 |
| A96 | 0.0070 |
| A97 | 0.0021 |
| A98 | 0.0019 |
| A99 | 0.0006 |
| A100 | 0.25 |
| A101 | 0.178 |
| A102 | 0.0001 |
| A103 | 0.156 |
| A104 | 0.309 |
| A105 | 0.51 |
| A106 | 0.17 |
| A107 | 0.001 |
| A108 | 0.4° |
| A109 | 0.17 |
| A110 | 0.0002 |
| A111 | 0.0028 |
| A112 | 0.1788 |
| A113 | 0.2411 |
| A114 | 0.0003 |
| A115 | 0.8049 |
| A116 | 0.0289 |
| A117 | 0.0028 |
| A119 | 0.00001 |
| A120 | <0.00001 |
| A121 | 0.018 |
| A122 | <0.0001 |
| A123 | 5.3 |
| A124 | 0.0001 |
| A125 | 0.0042 |
| A126 | <0.00001 |
| A127 | 0.01 |
| A128 | 3.95 |
| A129 | 0.13 |
| A130 | 0.088 |
| A131 | 0.0002 |
| A132 | 0.066 |
| A133 | 0.002 |
| A134 | 1.18 |
| A135 | 0.0022 |
| A136 | 0.083 |
| A137 | 0.0005 |
| A138 | 0.028 |
| A139 | 0.003 |
| A140 | 0.045 |
| A141 | 0.055 |
| A142 | 0.032 |
| A143 | 0.004 |
| A144 | 0.023 |
| A145 | 0.150 |
| A146 | 0.038 |
| A147 | 0.558 |
| A148 | 0.007 |
| A149 | 0.0001 |
| A150 | <0.0001 |
| A151 | 0.0002 |
| A152 | 0.0078 |
| A153 | 0.0006 |
| A154 | 0.334 |
| A155 | 0.611 |
| A156 | 0.289 |
| A157 | 0.0002 |
| A158 | 0.002 |
| A159 | 0.003 |
| A160 | 0.470 |
| A161 | 0.582 |
| A162 | 0.0000004 |
| A163 | 0.017 |
| A164 | 0.0000090 |
| A165 | 0.0000001 |
| A166 | 0.00250 |
| A167 | 0.000002 |
| A168 | 0.75 |
| A169 | 1.25 |
| A170 | 0.0002 |
| A171 | 0.0001 |
| A172 | 0.0185 |
| A173 | 0.0386 |
| A174 | 0.0014 |
| A175 | 1.357 |
| A176 | 0.002 |
| A177 | 1.670 |
| A178 | 1.036 |
| A179 | 0.006 |
| A180 | 0.095 |
| A181 | 0.520 |
| A182 | 0.001 |
| A183 | 0.019 |
| A185 | 0.910 |
| A186 | 0.319 |
| A187 | 0.00001 |
| A188 | 0.392 |
| A189 | 1.373 |
| A191 | 0.006 |
| A192 | 0.978 |
| A193 | 0.135 |
| A194 | 0.005 |
| A195 | 0.211 |
| A196 | 0.000006 |
| A197 | 0.027 |
| A198 | 0.006 |
| A199 | 0.494 |
| A200 | 0.0004 |
| A201 | 0.001 |
| A202 | 0.021 |
| A203 | 0.00000001 |
| A204 | 0.001 |
| A205 | 0.0002 |
| A206 | 0.0000004 |
| A207 | 0.764 |
| A208 | 0.004 |
| A209 | 0.00001 |
| A210 | 0.00001 |
| A211 | 0.504 |
| A212 | 4.148 |
| A213 | 5.016 |
| A214 | 1.855 |
| A215 | 1.009 |
| A216 | 0.006 |
| A217 | 0.010 |
| A218 | 0.001 |
| A219 | 0.160 |
| A220 | 0.183 |
| A221 | 0.00001 |
| A222 | 0.003 |
| A223 | 0.007 |
| A224 | 0.0001 |
| A226 | 0.062 |
| A227 | 0.412 |
| A228 | 0.00005 |
| A229 | 20.000 |
| A230 | 0.007 |
| A231 | 0.089 |
| A232 | 0.022 |
| A233 | 0.025 |
| A234 | 0.001 |
| A235 | 0.00003 |
| A236 | 0.817 |
| A237 | 0.003 |
| A238 | 1.165 |
| A239 | 0.00001 |
| A240 | 0.638 |
| A241 | 0.614 |
| A242 | 1.199 |
| A243 | 0.0004 |
| A248 | 0.0004 |
| A250 | 0.036 |
| A251 | 0.000 |
| A252 | 0.002 |
| A253 | 0.002 |
| A254 | 0.002 |
| A255 | 0.208 |
| A256 | 0.001 |
| A257 | 0.0001 |
| A258 | 0.0000001 |
| A259 | 0.413 |
| A260 | 0.002 |
| A261 | 1.087 |
| A262 | 0.203 |
| A263 | 7.02 |
| A264 | 0.00002 |
| A265 | 0.0000001 |
| A266 | 0.0001 |
| A267 | 0.009 |
| A268 | 0.00008 |
| A269 | 0.418 |
| A270 | 0.00005 |
| A271 | 0.936 |
| A272 | 0.001 |
| A273 | 0.000002 |
| A274 | 2.880 |
| A275 | 0.001 |
| A276 | 4.154 |
| A277 | 0.218 |
| A278 | 0.249 |
| A279 | 0.423 |
| A280 | 4.502 |
| A281 | 0.001 |
| A282 | 0.001 |
| A283 | 0.122 |
| A284 | 0.008 |
| A285 | 1.303 |
| A286 | 0.0001 |
| A287 | 1.063 |
| A288 | 0.260 |
| A289 | 0.00004 |
| A290 | 0.000001 |
| A291 | >20 |
| A292 | 0.159 |
| A293 | 16.120 |
| A294 | 0.486 |
| A295 | 0.00001 |
| A296 | 0.000001 |
| A297 | 0.000000006 |
| A298 | 0.000002 |
| A299 | 0.0000002 |
| A300 | 0.00004 |
| A301 | 0.03620 |
| A302 | 0.00007 |
| A303 | 0.055 |
| A304 | 0.00002 |
| A305 | 0.0004 |
| A306 | 4.41 |
| A307 | 4.44 |
| A308 | 0.000001 |
| A309 | 0.000001 |
| A310 | 0.00002 |
| A311 | 0.181 |
| A312 | 0.640 |
| A313 | 0.139 |
| A314 | 0.001 |
| A315 | 0.051 |
| A316 | 0.00003 |
| A317 | 0.0004 |
| A318 | 0.139 |
| A319 | 0.001 |
| A320 | 0.0001 |
| A321 | 0.0000001 |
| A322 | 1.722 |
| A323 | 3.839 |
| A324 | 0.0003 |
| A325 | 0.00000002 |
| A326 | 0.367 |
| A327 | 5.329 |

All references provided herein are incorporated herein in their entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A compound, or pharmaceutically acceptable salt thereof, having a structure of Formula (I): 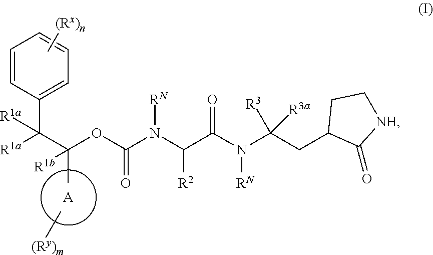
wherein
each R^{N} is independently H or C₁₋₆alkyl;
each R^{1a} is independently hydrogen, halo, C₁₋₆alkyl, or C₁₋₆haloalkyl,
or both R^{1a} with the carbon to which they are attached form a spiro C₃₋₆carbocyclyl or spiro 4-8-membered heterocyclyl having 1-3 ring heteroatoms selected from N, O, and S, which is optionally substituted with C(O)OR^{N};
R^{1b} is hydrogen, halo, hydroxyl, C₁₋₆alkyl, or C₁₋₆haloalkyl;
n is 0, 1, or 2;
each R^{x} is independently halo, C₁₋₆alkyl, C₃₋₆carbocyclyl, or C₆₋₁₀aryl, wherein the aryl is optionally substituted with 1 or 2 substituents independently selected from OH, halo, C₁₋₆alkyl, C₁₋₆haloalkyl, and C₁₋₆alkoxy;
m is 0, 1, or 2;
each R^{y} is independently halo or C₁₋₆alkyl;
R² is C₁₋₆alkyl, C₁₋₆haloalkyl, C₁₋₆alkylene-C₃₋₈carbocyclyl, or C₁₋₆alkylene-C₆₋₁₀aryl, wherein the aryl is optionally substituted with 1 or 2 substituents independently selected from OH, halo, C₁₋₆alkyl, C₁₋₆haloalkyl, and C₁₋₆alkoxy;
R³ is CN, C₁₋₆alkylene-O(O)C—C₁₋₆alkyl substituted with SO₃H, C₁₋₆alkenylene-C(O)O—C₁₋₆alkyl, C₁₋₆ alkylene-OH substituted with PO(OCH₂CH₂)₂, CHO, or —[C(O)]₂—NR^{N}—B, wherein B is C₁₋₆alkyl, C₁₋₆hydroxyalkyl, C₃₋₈carbocyclyl, or 4-12-membered heterocyclyl having 1-3 ring heteroatoms selected from N, O, and S, and the carbocyclyl or heterocyclyl is optionally mono-substituted with C₁₋₆alkyl;
R^{3a} is H or C₁₋₆alkyl; and
ring A is C₆₋₁₀cycloalkyl, C₆₋₁₀aryl, or 5-10 membered heteroaryl comprising one nitrogen heteroatom,
with the proviso that when each R^{1a} is hydrogen or each R^{1a} is methyl, ring A is phenyl, m is 0 or 1, R^{y} is halo, and n is 1, then R^{x} is other than chloro, and
with the proviso that the compound is not:
1,2-diphenylethyl (4-methyl-1-oxo-1-((1-oxo-3-(2-oxopyrrolidin-3-yl)propan-2-yl)amino)pentan-2-yl)carbamate;
2-(2-(((1,2-diphenylethoxy)carbonyl)amino)-4-methylpentanamido)-1-hydroxy-3-(2-oxopyrrolidin-3-yl)propane-1-sulfonic acid;
2-(3-chlorophenyl)-1-phenylethyl (4-methyl-1-oxo-1-((1-oxo-3-(2-oxopyrrolidin-3-yl)propan-2-yl)amino)pentan-2-yl)carbamate;
2-(3-chlorophenyl)-1-(3-fluorophenyl)-2-methylpropyl (3-cyclohexyl-1-oxo-1-((1-oxo-3-(2-oxopyrrolidin-3-yl)propan-2-yl)amino)propan-2-yl)carbamate;
1,2-bis(3-chlorophenyl)-2-methylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (4-methyl-1-oxo-1-((1-oxo-3-(2-oxopyrrolidin-3-yl)propan-2-yl)amino)pentan-2-yl)carbamate;
2-(3-chlorophenyl)-1-phenylethyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (1-((4-amino-3,4-dioxo-1-(2-oxopyrrolidin-3-yl)butan-2-yl)amino)-3-cyclohexyl-1-oxopropan-2-yl)carbamate;
2-(2-(((2-(3-chlorophenyl)-1-phenylethoxy)carbonyl)amino)-4-methylpentanamido)-1-hydroxy-3-(2-oxopyrrolidin-3-yl)propane-1-sulfonic acid;
2-(2-(((2-(3-chlorophenyl)-1-phenylethoxy)carbonyl)amino)-3-cyclohexylpropanamido)-1-hydroxy-3-(2-oxopyrrolidin-3-yl)propane-1-sulfonic acid;
2-(3-chlorophenyl)-1-phenylethyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-1-(3-fluorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-2-methyl-1-(naphthalen-2-yl)propyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-2-methyl-1-(m-tolyl)propyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-2-methyl-1-(naphthalen-2-yl)propyl (3-cyclohexyl-1-oxo-1-((1-oxo-3-(2-oxopyrrolidin-3-yl)propan-2-yl)amino)propan-2-yl)carbamate;
2-(3-chlorophenyl)-1-(4-chlorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-1-(4-fluorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-2-methyl-1-(m-tolyl)propyl (3-cyclohexyl-1-oxo-1-((1-oxo-3-(2-oxopyrrolidin-3-yl)propan-2-yl)amino)propan-2-yl)carbamate;
2-(3-chlorophenyl)-1-(4-fluorophenyl)-2-methylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-1-(4-chlorophenyl)-2-methylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-1-(4-fluorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
2-(3-chlorophenyl)-1-(4-chlorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
2-(3-chlorophenyl)-1-phenylethyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
2-(3-chlorophenyl)-2-methyl-1-phenylpropyl (1-oxo-1-((1-oxo-3-(2-oxopyrrolidin-3-yl)propan-2-yl)amino)hexan-2-yl)carbamate;
2-(3-chlorophenyl)-1-phenylethyl (3-cyclohexyl-1-((4-(cyclopropylamino)-3,4-dioxo-1-(2-oxopyrrolidin-3-yl)butan-2-yl)amino)-1-oxopropan-2-yl)carbamate;
1-(2-chlorophenyl)-2-(3-chlorophenyl)-2-methylpropyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (1-((4-amino-3,4-dioxo-1-(2-oxopyrrolidin-3-yl)butan-2-yl)amino)-1-oxohexan-2-yl)carbamate;
2-(3-chlorophenyl)-2-methyl-1-phenylpropyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (1-((4-(cyclopropylamino)-3,4-dioxo-1-(2-oxopyrrolidin-3-yl)butan-2-yl)amino)-1-oxohexan-2-yl)carbamate;
2-(3-chlorophenyl)-1-(3-fluorophenyl)-2-methylpropyl (1-((4-amino-3,4-dioxo-1-(2-oxopyrrolidin-3-yl)butan-2-yl)amino)-3-cyclohexyl-1-oxopropan-2-yl)carbamate;
2-(3-chlorophenyl)-1-(3-fluorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
1-(2-chlorophenyl)-2-(3-chlorophenyl)-2-methylpropyl (3-cyclohexyl-1-((4-(cyclopropylamino)-3,4-dioxo-1-(2-oxopyrrolidin-3-yl)butan-2-yl)amino)-1-oxopropan-2-yl)carbamate;
(3-chlorophenyl)(1-(3-chlorophenyl)cyclopropyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(4-chlorophenyl)(1-(3-chlorophenyl)cyclopropyl)methyl (4-methyl-1-oxo-1-((1-oxo-3-(2-oxopyrrolidin-3-yl)propan-2-yl)amino)pentan-2-yl)carbamate;
(4-chlorophenyl)(1-(3-chlorophenyl)cyclopropyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(3-chlorophenyl)(1-(3-chlorophenyl)cyclopropyl)methyl (4-methyl-1-oxo-1-((1-oxo-3-(2-oxopyrrolidin-3-yl)propan-2-yl)amino)pentan-2-yl)carbamate;
2-(3-chlorophenyl)-2-methyl-1-phenylpropyl (1-((4-amino-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (3-cyclohexyl-1-((4-(ethylamino)-3,4-dioxo-1-(2-oxopyrrolidin-3-yl)butan-2-yl)amino)-1-oxopropan-2-yl)carbamate;
(3-chlorophenyl)(1-(3-chlorophenyl)cyclopropyl)methyl (3-cyclohexyl-1-((4-(cyclopropylamino)-3,4-dioxo-1-(2-oxopyrrolidin-3-yl)butan-2-yl)amino)-1-oxopropan-2-yl)carbamate;
2-(3-chlorophenyl)-2-methyl-1-(naphthalen-2-yl)propyl (3-cyclohexyl-1-((4-(diethylamino)-3,4-dioxo-1-(2-oxopyrrolidin-3-yl)butan-2-yl)amino)-1-oxopropan-2-yl)carbamate;
2-(3-chlorophenyl)-1-(4-chlorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate;
ethyl (E)-4-(2-(((1,2-diphenylethoxy)carbonyl)amino)-4-methylpentanamido)-5-(2-oxopyrrolidin-3-yl)pent-2-enoate;
3-(2-(((2-(3-chlorophenyl)-2-methyl-1-(naphthalen-2-yl)propoxy)carbonyl)amino)-3-cyclohexylpropanamido)-2-oxo-4-(2-oxopyrrolidin-3-yl)butanoic acid;
2-(3-chlorophenyl)-1-(4-fluorophenyl)-2-methylpropyl (4-methyl-1-oxo-1-((1-oxo-3-(2-oxopyrrolidin-3-yl)propan-2-yl)amino)pentan-2-yl)carbamate;
1,2-bis(3-chlorophenyl)ethyl (3-cyclohexyl-1-((4-(cyclopropylamino)-3,4-dioxo-1-(2-oxopyrrolidin-3-yl)butan-2-yl)amino)-1-oxopropan-2-yl)carbamate;
1,2-bis(3-chlorophenyl)ethyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-1-(4-chlorophenyl)-2-methylpropyl (4-methyl-1-oxo-1-((1-oxo-3-(2-oxopyrrolidin-3-yl)propan-2-yl)amino)pentan-2-yl)carbamate;
2-(3-chlorophenyl)-1-(4-fluorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate;
(3-chlorophenyl)(1-(3-chlorophenyl)cyclopropyl)methyl (3-cyclohexyl-1-oxo-1-((1-oxo-3-(2-oxopyrrolidin-3-yl)propan-2-yl)amino)propan-2-yl)carbamate;
2-(3-chlorophenyl)-2,2-difluoro-1-phenylethyl (1-((4-amino-3,4-dioxo-1-(2-oxopyrrolidin-3-yl)butan-2-yl)amino)-4-methyl-1-oxopentan-2-yl)carbamate;
2-(3-chlorophenyl)-2,2-difluoro-1-phenylethyl (4-methyl-1-oxo-1-((1-oxo-3-(2-oxopyrrolidin-3-yl)propan-2-yl)amino)pentan-2-yl)carbamate;
2-(3-chlorophenyl)-2-methyl-1-phenylpropyl (1-((4-amino-3,4-dioxo-1-(2-oxopyrrolidin-3-yl)butan-2-yl)amino)-1-oxo-3-phenylpropan-2-yl)carbamate;
2-(3-chlorophenyl)-2-methyl-1-phenylpropyl (1-oxo-1-((1-oxo-3-(2-oxopyrrolidin-3-yl)propan-2-yl)amino)-3-phenylpropan-2-yl)carbamate;
2-(3-chlorophenyl)-2,2-difluoro-1-phenylethyl (1-((4-(cyclopropylamino)-3,4-dioxo-1-(2-oxopyrrolidin-3-yl)butan-2-yl)amino)-4-methyl-1-oxopentan-2-yl)carbamate;
1,2-bis(3-chlorophenyl)-2,2-difluoroethyl (4-methyl-1-oxo-1-((1-oxo-3-(2-oxopyrrolidin-3-yl)propan-2-yl)amino)pentan-2-yl)carbamate;
2-(3-chlorophenyl)-2-methyl-1-phenylpropyl (1-oxo-1-((1-oxo-3-(2-oxopyrrolidin-3-yl)propan-2-yl)amino)heptan-2-yl)carbamate;
2-(3-chlorophenyl)-1-(3-fluorophenyl)-2-methylpropyl (4-methyl-1-oxo-1-((1-oxo-3-(2-oxopyrrolidin-3-yl)propan-2-yl)amino)pentan-2-yl)carbamate;
1,2-bis(3-chlorophenyl)ethyl (3-cyclohexyl-1-oxo-1-((1-oxo-3-(2-oxopyrrolidin-3-yl)propan-2-yl)amino)propan-2-yl)carbamate;
2-(3-chlorophenyl)-2-methyl-1-phenylpropyl (4-methyl-1-oxo-1-((1-oxo-3-(2-oxopyrrolidin-3-yl)propan-2-yl)amino)pentan-2-yl)carbamate;
(1-(3-chlorophenyl)cyclobutyl)(phenyl)methyl (4-methyl-1-oxo-1-((1-oxo-3-(2-oxopyrrolidin-3-yl)propan-2-yl)amino)pentan-2-yl)carbamate;
(1-(3-chlorophenyl)cyclopentyl)(phenyl)methyl (4-methyl-1-oxo-1-((1-oxo-3-(2-oxopyrrolidin-3-yl)propan-2-yl)amino)pentan-2-yl)carbamate;
1,2-bis(3-chlorophenyl)ethyl (4-methyl-1-oxo-1-((1-oxo-3-(2-oxopyrrolidin-3-yl)propan-2-yl)amino)pentan-2-yl)carbamate;
2-(3-chlorophenyl)-2-methyl-1-phenylpropyl (1-((4-(cyclopropylamino)-3,4-dioxo-1-(2-oxopyrrolidin-3-yl)butan-2-yl)amino)-1-oxo-3-phenylpropan-2-yl)carbamate;
2-(3-chlorophenyl)-1-(3-fluorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate;
1,2-bis(3-chlorophenyl)-2,2-difluoroethyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (1-((4-(cyclopropylamino)-3,4-dioxo-1-(2-oxopyrrolidin-3-yl)butan-2-yl)amino)-1-oxoheptan-2-yl)carbamate;
(1-(3-chlorophenyl)cyclohexyl)(phenyl)methyl (4-methyl-1-oxo-1-((1-oxo-3-(2-oxopyrrolidin-3-yl)propan-2-yl)amino)pentan-2-yl)carbamate;
(1-(3-chlorophenyl)cyclopropyl)(4-fluorophenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(4-chlorophenyl)(1-(3-chlorophenyl)cyclopentyl)methyl (4-methyl-1-oxo-1-((1-oxo-3-(2-oxopyrrolidin-3-yl)propan-2-yl)amino)pentan-2-yl)carbamate;
(1-(3-chlorophenyl)cyclobutyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-ethyl-1-phenylbutyl (4-methyl-1-oxo-1-((1-oxo-3-(2-oxopyrrolidin-3-yl)propan-2-yl)amino)pentan-2-yl)carbamate;
2-(3-fluorophenyl)-2-methyl-1-phenylpropyl (1-((4-(cyclopropylamino)-3,4-dioxo-1-(2-oxopyrrolidin-3-yl)butan-2-yl)amino)-4-methyl-1-oxopentan-2-yl)carbamate;
(1-(3-chlorophenyl)cyclopentyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(4-chlorophenyl)(1-(3-chlorophenyl)cyclopentyl)methyl (1-((4-(cyclopropylamino)-3,4-dioxo-1-(2-oxopyrrolidin-3-yl)butan-2-yl)amino)-4-methyl-1-oxopentan-2-yl)carbamate;
2-(3-chlorophenyl)-2-methyl-1-phenylpropyl (4-methyl-1-((4-((1-methylazetidin-3-yl)amino)-3,4-dioxo-1-(2-oxopyrrolidin-3-yl)butan-2-yl)amino)-1-oxopentan-2-yl)carbamate;
2-(3-chlorophenyl)-2-ethyl-1-(4-fluorophenyl)butyl (4-methyl-1-oxo-1-((1-oxo-3-(2-oxopyrrolidin-3-yl)propan-2-yl)amino)pentan-2-yl)carbamate;
(3-chlorophenyl)(1-(3-chlorophenyl)cyclopentyl)methyl (4-methyl-1-oxo-1-((1-oxo-3-(2-oxopyrrolidin-3-yl)propan-2-yl)amino)pentan-2-yl)carbamate;
(3-chlorophenyl)(1-(3-chlorophenyl)cyclopentyl)methyl (1-((4-(cyclopropylamino)-3,4-dioxo-1-(2-oxopyrrolidin-3-yl)butan-2-yl)amino)-4-methyl-1-oxopentan-2-yl)carbamate;
1,2-bis(3-chlorophenyl)-2-methylpropyl (1-((4-(cyclopropylamino)-3,4-dioxo-1-(2-oxopyrrolidin-3-yl)butan-2-yl)amino)-4-methyl-1-oxopentan-2-yl)carbamate;
(1-(3-chlorophenyl)cyclopropyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(1-(3-chlorophenyl)cyclopropyl)(4-fluorophenyl)methyl (4-methyl-1-oxo-1-((1-oxo-3-(2-oxopyrrolidin-3-yl)propan-2-yl)amino)pentan-2-yl)carbamate;
(1-(3-fluorophenyl)cyclopropyl)(phenyl)methyl (4-methyl-1-oxo-1-((1-oxo-3-(2-oxopyrrolidin-3-yl)propan-2-yl)amino)pentan-2-yl)carbamate;
(1-(3-fluorophenyl)cyclopropyl)(phenyl)methyl (1-((4-(cyclopropylamino)-3,4-dioxo-1-(2-oxopyrrolidin-3-yl)butan-2-yl)amino)-4-methyl-1-oxopentan-2-yl)carbamate;
(4-chlorophenyl)(1-(3-chlorophenyl)cyclobutyl)methyl (4-methyl-1-oxo-1-((1-oxo-3-(2-oxopyrrolidin-3-yl)propan-2-yl)amino)pentan-2-yl)carbamate; or
(4-chlorophenyl)(1-(3-chlorophenyl)cyclobutyl)methyl (1-((4-(cyclopropylamino)-3,4-dioxo-1-(2-oxopyrrolidin-3-yl)butan-2-yl)amino)-4-methyl-1-oxopentan-2-yl)carbamate.

2. The compound or salt of claim 1, wherein each R^{N} is H.

3. The compound or salt of claim 1 or 2, wherein at least one R^{1a} is hydrogen.

4. The compound or salt of any one of claims 1 to 3, wherein at least one R^{1a} is halo.

5. The compound or salt of claim 4, wherein each R^{1a} is halo.

6. The compound or salt of claim 4 or 5, wherein halo is chloro or fluoro.

7. The compound or salt of any claim 4 or 5, wherein halo is fluoro.

8. The compound or salt of claims 1 to 4, 6, and 7, wherein at least one R^{1a} is C₁₋₆alkyl.

9. The compound or salt of claim 8, wherein each R^{1a} is C₁₋₆ alkyl.

10. The compound or salt of claim 8 or 9, wherein C₁₋₆alkyl is methyl or ethyl.

11. The compound or salt of claim 1 or 2, wherein each R^{1a}, together with the carbon to which they are attached, form a spiro C₃₋₆carbocyclyl.

12. The compound or salt of claim 11, wherein the C₃₋₆carbocyclyl is cyclopropyl, cyclobutyl, or cyclohexyl.

13. The compound or salt of claim 12, wherein the C₃₋₆carbocyclyl is cyclopropyl.

14. The compound or salt of claim 12, wherein the C₃₋₆carbocyclyl is cyclobutyl.

15. The compound or salt of claim 12, wherein the C₃₋₆carbocyclyl is cyclohexyl.

16. The compound or salt of any one of claims 1 to 15, wherein n is 0.

17. The compound or salt of any one of claims 1 to 15, wherein n is 1.

18. The compound or salt of claim 17, wherein R^{x} is in a meta position.

19. The compound or salt of claim 17 or 18, wherein R^{x} is halo.

20. The compound or salt of claim 19, wherein R^{x} is chloro.

21. The compound or salt of claim 19, wherein R^{x} is fluoro.

22. The compound or salt of any one of claims 1 to 15, wherein n is 2.

23. The compound or salt of claim 22, wherein at least one R^{x} is halo.

24. The compound or salt of any one of claims 1 to 23, wherein m is 0.

25. The compound or salt of any one of claims 1 to 23, wherein m is 1.

26. The compound or salt of claim 25, wherein R^{y} is in a meta position.

27. The compound or salt of claim 25, wherein R^{y} is in a para position.

28. The compound or salt of any one of claims 25 to 27, wherein R^{y} is halo.

29. The compound or salt of claim 28, wherein halo is chloro.

30. The compound or salt of claim 28, wherein halo is fluoro.

31. The compound or salt of any one of claims 1 to 23, wherein m is 2.

32. The compound or salt of claim 31, wherein one R^{y} is in a meta position.

33. The compound or salt of claim 31 or 32, wherein one R^{y} is in a para position.

34. The compound or salt of any one of claims 31 to 33, wherein at least one R^{y} is halo.

35. The compound or salt of claim 34, wherein each R^{y} is halo.

36. The compound or salt of claim 34 or 35, wherein at least one halo is fluoro.

37. The compound or salt of claim 36, wherein each R^{y} is fluoro.

38. The compound or salt of any one of claims 34 to 36, wherein at least one halo is chloro.

39. The compound or salt according to any one of claims 1 to 25, wherein R² is C₁₋₆alkyl.

40. The compound or salt according to any one of claims 1 to 39, wherein R² is

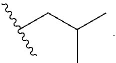 [SMILES: *CC(C)C]

41. The compound or salt according to any one of claims 1 to 39, wherein R² is C₁₋₆alkylene-C₅₋₈carbocyclyl.

42. The compound or salt according to claim 41, wherein R² is

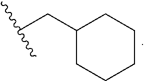 [SMILES: *CC1CCCCC1]

43. The compound or salt according to any one of claims 1 to 42, wherein R³ is CHO.

44. The compound or salt according to any one of claims 1 to 42, wherein R³ is —[C(O)]₂—NR^{N}—B.

45. The compound or salt according to claim 44, wherein B is C₃₋₆carbocyclyl optionally mono-substituted with C₁₋₆ alkyl.

46. The compound or salt according to claim 45, wherein B is C₃carbocyclyl optionally mono-substituted with C₁₋₆ alkyl.

47. The compound or salt according to claim 44, wherein B is 4-12-membered heterocyclyl having 1-3 ring heteroatoms selected from N, O, and S optionally mono-substituted with C₁₋₆alkyl.

48. The compound or salt according to claim 47, wherein B comprises piperidinyl optionally mono-substituted with C₁₋₆alkyl.

49. The compound or salt according to claim 1, wherein ring A comprises phenyl, each R^{1a} is F, R^{1b} is H, each R^{N} is H, n is 1, R^{x} is halo, R² is C₁₋₆haloalkyl, C₁₋₆alkylene-C₃₋₈carbocyclyl, or C₁₋₆alkylene-C₆₋₁₀aryl, wherein the aryl is optionally substituted with 1 or 2 substituents independently selected from OH, halo, C₁₋₆alkyl, C₁₋₆ haloalkyl, and C₁₋₆alkoxy; R³ is CN, C₁₋₆alkylene-O(O)C—C₁₋₆alkyl substituted with SO₃H, C₁₋₆alkenylene-C(O)O—C₁₋₆alkyl, C₁₋₆alkylene-OH substituted with PO(OCH₂CH₂)₂, CHO, or —[C(O)]₂—NR^{N}—B.

50. The compound or salt according to any one of claims 1 to 49, having a structure of Formula (Ia): 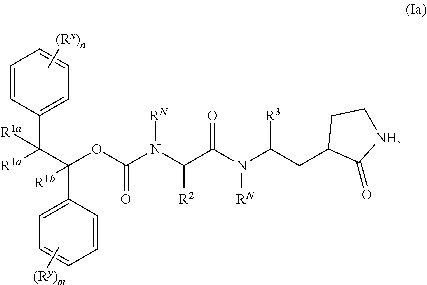
wherein
each R^{N} is independently H or C₁₋₆alkyl;
each R^{1a} is independently hydrogen, halo, C₁₋₆alkyl, or C₁₋₆haloalkyl, or both R^{1a} with the carbon to which they are attached form a spiro C₃₋₆carbocyclyl;
R^{1b} is hydrogen, halo, C₁₋₆alkyl, or C₁₋₆haloalkyl;
n is 0, 1, or 2;
each R^{x} is independently halo or C₁₋₆alkyl;
m is 0, 1, or 2;
each R^{y} is independently halo or C₁₋₆alkyl;
R² is C₁₋₆alkyl or C₁₋₆alkylene-C₅₋₈carbocyclyl; and
R³ is CHO or —[C(O)]₂—NR^{N}—B, wherein B is C₁₋₆alkyl, C₃₋₆carbocyclyl, or 4-12-membered heterocyclyl having 1-3 ring heteroatoms selected from N, O, and S, and the carbocyclyl or heterocyclyl is optionally mono-substituted with C₁₋₆alkyl.

51. A compound or pharmaceutically acceptable salt thereof, having a structure of Formula (Ib): 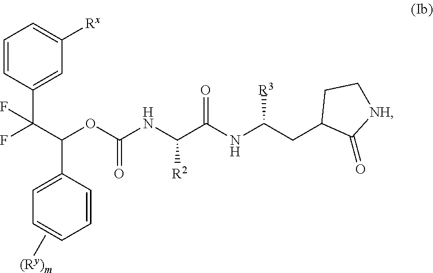
wherein
R^{x} is Cl, F, cyclopropyl, or phenyl;
R^{y} is F;
m is 0 or 1;
R² is C₄₋₅ alkyl, C₃₋₅haloalkyl, CH₂Cl₃₋₆carbocyclyl, or benzyl, and the carbocyclyl is optionally substituted with methyl or ethyl; and
R³ is CHO, C(O)C(O)NH₂, C(O)C(O)NHcyclopropyl, or C(O)C(O)NHethyl,
with the proviso that when R^{x} is Cl, m is 0, and R³ is CHO, C(O)C(O)NH₂, or C(O)C(O)NHcyclopropyl, then R² is not 2-methyl-propyl.

52. The compound or salt of claim 51, wherein R² is 2-methyl-propyl, butyl, pentyl, 2-methyl-butyl, 3,3-difluoropropyl, CH₂-cyclopropyl, CH₂cyclobutyl, CH₂cyclopentyl, CH₂-cyclohexyl, CH₂-(1-ethylcyclopropyl), CH₂-(1-methylcyclobutyl), CH₂-(1-ethylcyclobutyl), CH₂-(1-ethylcyclopentyl), or benzyl.

53. The compound or salt of claim 52, wherein R² is butyl, pentyl, 2-methyl-butyl, 3,3-difluoropropyl, CH₂-cyclopropyl, CH₂cyclobutyl, CH₂cyclopentyl, CH₂-cyclohexyl, CH₂-(1-ethylcyclopropyl), CH₂-(1-methylcyclobutyl), CH₂-(1-ethylcyclobutyl), CH₂-(1-ethylcyclopentyl), or benzyl.

54. The compound or salt of any one of claims 51 to 53, wherein R^{x} is Cl.

55. The compound or salt of any one of claims 51 to 54, wherein m is 0.

56. The compound or salt of any one of claims 51 to 54, wherein R^{y} is F, or more specifically meta-F.

57. The compound or salt of any one of claims 51 to 56, wherein R² is CH₂cyclohexyl.

58. The compound or salt of any one of claims 51 to 56, wherein R² is butyl or 2-methyl-butyl.

59. The compound or salt of any one of claims 51 to 58, wherein R³ is CHO.

60. The compound or salt of any one of claims 51 to 58, wherein R³ is C(O)C(O)NHcyclopropyl.

61. A compound as recited in Table A or Table B, or a pharmaceutically acceptable salt thereof.

62. The compound or salt of claim 61, selected from Compounds A20 and A25-A29.

63. The compound or salt of claim 61, selected from A20, A20-1, A20-2, A25-1, A48, A72, A119, A126, A164, A165, A167, A187, A196, A206, A209, A210, A228, A235, A239, A258, A265, A268, A270, A273, A289, A290, A295, A296, A297, A298, A299, A300, A302, A308, A309, A310, A316, A321, and A325.

64. A pharmaceutical formulation comprising the compound or salt according to any one of claims 1 to 63 and a pharmaceutically acceptable excipient.

65. A method for treating or preventing a viral infection in a host, comprising administering to the host a therapeutic amount of the compound or salt according to any one of claims 1 to 63.

66. The method according to claim 65, wherein the viral infection is coronavirus infection, calicivirus infection, or picornavirus infection.

67. The method according to claim 66, wherein the viral infection is a calicivirus infection.

68. The method according to claim 67, wherein the calicivirus infection is a norovirus infection.

69. The method according to claim 66, wherein the viral infection is a picornavirus infection.

70. The method according to claim 69, wherein the picornavirus infection is rhinovirus infection.

71. The method according to claim 70, wherein the rhinovirus infection is a rhinovirus A, rhinovirus B, or rhinovirus C infection.

72. The method according to claim 66, wherein the viral infection is a coronavirus infection.

73. The method according to claim 72, wherein the coronavirus infection is severe acute respiratory syndrome (SARS), Middle East respiratory syndrome (MERS), or Coronavirus disease 2019 (COVID-19).

74. The method according to claim 72, wherein the coronavirus is an alphacoronavirus.

75. The method according to claim 72, wherein the coronavirus is a betacoronavirus.
